(19) United States
(12) Patent Application Publication
(10) Pub. No.: US 2015/0073188 A1
(43) Pub. Date: Mar. 12, 2015

(54) PROCESSES FOR PRODUCING SYNTHETIC HYDROCARBONS FROM COAL, BIOMASS, AND NATURAL GAS

(71) Applicant: The Trustees of Princeton University, Princeton, NJ (US)

(72) Inventors: Christodoulos A. Floudas, Princeton, NJ (US); Richard C. Baliban, Southampton, NJ (US); Josephine A. Elia, Plainsboro, NJ (US)

(21) Appl. No.: 14/377,759

(22) PCT Filed: Mar. 1, 2013
(86) PCT No.: PCT/US13/28730

§ 371 Date: Aug. 8, 2014

Related U.S. Application Data
(60) Provisional application No. 61/605,547, filed on Mar. 1, 2012.
(60) Provisional application No. 61/716,348, filed on Oct. 19, 2012.

(51) Int. Cl. G06F 17/50; C10G 69/14; C07C 5/27; G06F 17/11; C07C 2/76; C10L 1/04; C10L 1/06; C10L 1/08; C07C 1/22; C07C 5/03

(52) U.S. Cl. CPC G06F 17/5004; C07C 1/22; C10G 69/14; C07C 5/2767; C07C 5/03; C07C 2/76; C10L 1/04; C10L 1/06; C10L 1/08; G06F 17/11; C10L 2290/42; C10L 2290/02; C10L 2270/023; C10L 2270/026; C10L 2200/0492
USPC 585/332; 422/187; 585/640; 208/79; 585/310; 703/1

(57) ABSTRACT

Methods of optimal refinery design utilizing a thermochemical based superstructure are provided. Methods of producing liquid fuels utilizing a refinery selected from a thermochemical based superstructure are provided. Thermochemical based superstructures are provided. Refineries are provided.

This application claims the benefit of U.S. Provisional Application Nos. 61/605,547, filed Mar. 1, 2012, and 61/716,348, filed Oct. 19, 2012, both of which are incorporated herein by reference as if fully set forth.

This invention was made with government support under Grant No. EFRI-0937706 awarded by the National Science Foundation. The government has certain rights in the invention.

### FIELD

The disclosure herein relates to methods of converting coal, biomass or natural gas feedstocks into synthetic liquid hydrocarbons and processes for converting natural gas to synthetic liquid hydrocarbons.

### BACKGROUND

The challenges to reduce dependence on petroleum as energy sources, coupled with efforts to reduce greenhouse gas (GHG) emissions, are exigent problems faced by the US transportation sector. Several studies have been done to explore alternative, non-petroleum based processes to produce liquid fuels that include the production of synthetic liquid hydrocarbons from biomass, coal, and natural gas using a synthesis gas (syngas) intermediate. These energy processes have emerged as viable options to address the given challenges due to their capabilities to produce liquid fuels via domestically available sources of carbon-based energy. A common feature of these synthetic processes, however, is the large CO₂ amount emitted from the system.

In 2008, the United States consumed an average of 19498 thousand barrels of oil per day (TBD), including 11114 TBD of imports. The 2008 transportation sector requirement of 13702 TBD accounted for 70.2% of the total U.S. consumption. While it is estimated that liquid fuel use in residential, commercial, industrial, and electric power sectors will all decrease, on average, over the next 20 years, the anticipated average annual increase in the transportation sector requirement of 0.6% forecasts an increase in the total U.S. liquid fuels consumption. Because domestic oil production is expected to decline over this time period, the United States will ultimately require an increase in the percentage of oil consumed by foreign imports.

Although Canada and Mexico are two of the three largest foreign suppliers with 2493 and 1302 TBD of oil supplied to the United States in 2008 respectively, these two countries only have 3.2% of the proven global foreign oil reserves. This fact may prompt the United States to seek increased imports from Saudi Arabia and other Middle Eastern countries who have a combined 59.9% of the proven world reserves. However, turmoil within the Middle East and strained diplomatic relations can have a dramatic effect both on the availability and price of petroleum from this region. Furthermore, the increased energy usage of industrialized nations coupled with the rapid growth of China and India is likely to rapidly raise petroleum demand, which will result in an increase in the crude oil price. Therefore, it is imperative that the United States research, develop, and implement different methods for meeting transportation fuel demands using alternative processes.

A further concern with the increased use of transportation fuels is its contribution to the greenhouse gas (GHG) emissions. The transportation sector accounted for 33.0% of the CO₂ emissions in 2007, due almost exclusively to the direct consumption of fossil fuels. Although extensive research has been devoted to the use of alternative fuels such as hydrogen and electricity, so far, the technical and economic challenges have limited their widespread use.

Several technologies have been considered for the development of liquid fuels using biological feedstocks, including cellulosic and corn-based ethanol, soy-based biodiesel, and Fischer-Tropsch (FT) hydrocarbon fuels. The overall impact that each bio-based feedstock will have on displacing petroleum-based transportation fuels depends on the scale of production, the potential for rural economic development, the reduction in GHG emissions, the impact on soil fertility and agricultural ecology, the water use efficiency, and the costs associated with the upkeep, harvest, and transportation of the crop. The use of corn, soybean oil, and other vegetable oils as a feedstock for fuel production has led to concern regarding the impact on the price and availability of these substances as sources of food. In addition, the actual well-to-wheel GHG emissions from a corn-based ethanol fuel is not much of an improvement, compared to the emissions from gasoline or biodiesel. Bio-based feedstocks can still play a major role in satisfying transportation demands if the feedstock does not displace land that would otherwise be used for growing food crops and if the environmental impact of the feedstock production is minimized. Agricultural and forestry residues, waste products, and dedicated fuel crops are expected to be the dominant bio-based resources, but continuing analysis is required to develop a holistic approach to the sustainable production of transportation fuels from these feedstocks.

### SUMMARY

In an aspect the invention relates to a superstructure for forming a refinery. The superstructure includes at least one synthesis gas production unit configured to produce at least one synthesis gas selected from the group consisting of a biomass synthesis gas production unit, a coal synthesis gas production unit and a natural gas synthesis gas production unit, wherein the at least one synthesis gas is determined by a mixed-integer linear optimization model solved by a global optimization framework. The superstructure also includes a synthesis gas cleanup unit configured to remove undesired gases from the at least one synthesis gas, a liquid fuels production unit selected from the group consisting of a Fischer-Tropsch unit, and a methanol synthesis unit. The Fischer-Tropsch unit is configured to produce a first output from the at least one synthesis gas. The methanol synthesis unit is configured to produce a second output from the at least one synthesis gas. The selection of liquid fuels production unit is determined by the mixed-integer linear optimization model solved by the global optimization framework. The superstructure also includes a liquid fuels upgrading unit configured to upgrade the first output or the second output. The liquid fuels upgrading unit selection is determined by the mixed-integer linear optimization model solved by the global optimization framework. The superstructure also includes a hydrogen production unit configured to produce hydrogen for the refinery, an oxygen production unit configured to produce oxygen for the refinery, and a wastewater treatment network configured to process wastewater from the refinery and input freshwater into the refinery. The wastewater treatment network is determined by a mixed-integer linear optimization model solved by a global optimization framework. The superstructure also includes a utility plant configured to produce electricity for the refinery and process heat from the refinery. The utility plant is determined by a mixed-integer linear optimization model solved by a global optimization framework. The superstructure also includes a CO₂ separation unit configured to recycle gases containing CO₂ in the refinery. The at least one synthesis gas production unit, the synthesis gas cleanup unit, the liquid fuels production unit, the liquid fuels upgrading unit, the hydrogen production unit, the oxygen production unit, the wastewater treatment network, the utility plant and the CO₂ separation unit are configured to be combined to form the refinery.

In an aspect, the invention relates to an optimal refinery design system. The optimal refinery design system includes a superstructure database. The superstructure database includes data associated with at least one synthesis gas production unit configured to produce at least one synthesis gas selected from the group consisting of a biomass synthesis gas production unit, a coal synthesis gas production unit and a natural gas synthesis gas production unit. The selection of the at least one synthesis gas is determined by a mixed-integer linear optimization model solved by a global optimization framework. The superstructure database also includes data associated with a synthesis gas cleanup unit configured to remove undesired gases from the at least one synthesis gas. The superstructure also includes data associated with a liquid fuels production unit configured selected from the group consisting of a Fischer-Tropsch unit and a methanol synthesis unit. The Fischer-Tropsch unit is configured to produce a first output from the at least one synthesis gas, and the methanol synthesis unit is configured to produce a second output from the at least one synthesis gas. The selection of liquid fuels production unit is determined by the mixed-integer linear optimization model solved by the global optimization framework. The superstructure database also includes data associated with a liquid fuels upgrading unit configured to upgrade the first output or the second output. The liquid fuels upgrading unit is determined by the mixed-integer linear optimization model solved by the global optimization framework. The superstructure also includes data associated with a hydrogen production unit configured to produce hydrogen for the refinery, an oxygen production unit configured to produce oxygen for the refinery, and a wastewater treatment network configured to process wastewater from the refinery and input freshwater into the refinery. The wastewater treatment network is determined by the mixed-integer linear optimization model solved by the global optimization framework. The superstructure database also includes data associated with a utility plant configured to produce electricity for the refinery and process heat from the refinery. The utility plant is determined by the mixed-integer linear optimization model solved by the global optimization framework. The superstructure database also includes data associated with a CO₂ separation unit configured to recycle gases containing CO₂ in the refinery. The at least one synthesis gas production unit, the synthesis gas cleanup unit, the liquid fuels production unit, the liquid fuels upgrading unit, the hydrogen production unit, the oxygen production unit, the wastewater treatment network, the utility plant and the CO₂ separation unit are configured to be combined to form the refinery. The optimal refinery design system includes a processor configured to solve the mixed-integer linear optimization model by the global optimization framework.

In an aspect the invention relates to a method of designing an optimal refinery. The method includes providing any superstructure contained herein, inserting a data set on each of the each of the at least one synthesis gas production unit, the liquid fuels production unit, the liquid fuels upgrading unit, the wastewater treatment network and the utility plant into the mixed-integer linear optimization model. The method also includes solving the mixed-integer linear optimization model by the global optimization framework, and thereby determining each of the at least one synthesis gas production unit, the liquid fuels production unit, the liquid fuels upgrading unit, the wastewater treatment network and the utility plant to produce an optimal refinery design.

In an aspect, the invention relates to a method of designing an optimal refinery. The method includes providing a superstructure database, solving the mixed-integer linear optimization model by the global optimization framework, and thereby determining each of the at least one synthesis gas production unit, the liquid fuels production unit, the liquid fuels upgrading unit, the wastewater treatment network and the utility plant to produce an optimal refinery design.

In an aspect, the invention relates to a method of producing liquid fuels. The method includes producing liquid fuels with a refinery having an optimal refinery design. The optimal refinery design is obtained by providing any superstructure contained herein, inserting a data set on each of the each of the at least one synthesis gas production unit, the liquid fuels production unit, the liquid fuels upgrading unit, the wastewater treatment network and the utility plant into the mixed-integer linear optimization model. The method also includes solving the mixed-integer linear optimization model by the global optimization framework, determining each of the at least one synthesis gas production unit, the liquid fuels production unit, the liquid fuels upgrading unit, the wastewater treatment network and the utility plant to produce the optimal refinery design.

In an aspect, the invention relates to a method of producing liquid fuels. The method includes providing a superstructure database, solving the mixed-integer linear optimization model by the global optimization framework, determining each of the at least one synthesis gas production unit, the liquid fuels production unit, the liquid fuels upgrading unit, the wastewater treatment network and the utility plant to produce an optimal refinery design, and producing liquid fuels by the optimal refinery design.

In an aspect, the invention relates to any superstructure as shown and/or described herein and in the accompanying drawings.

In an aspect, the invention relates to any refinery design as shown and/or described herein and in the accompanying drawings.

In an aspect, the invention relates to any method of designing a refinery as shown and/or described herein and in the accompanying drawings.

In an aspect, the invention relates to any method of producing liquid fuels as shown and/or described herein and in the accompanying drawings.

In an aspect, the invention relates to a refinery having any refinery design as shown and/or described herein and in the accompanying drawings.

### BRIEF DESCRIPTION OF THE DRAWINGS

The following detailed description of the embodiments of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 illustrates an example topological superstructure.

FIG. 2 illustrates an example of biomass synthesis gas generation.

FIG. 3 illustrates an example of coal synthesis gas generation.

FIG. 4 illustrates an example of natural gas synthesis gas generation.

FIG. 5 illustrates an example of a synthesis gas cleaning section.

FIG. 6 illustrates an example liquid fuels production section.

FIG. 7 illustrates an example Fischer-Tropsch synthesis section.

FIG. 8 illustrates refinery hydrogen and oxygen production.

FIG. 9 illustrates an example of combined heat, power, and water integration.

FIG. 10 illustrates a topological superstructure.

FIG. 11 illustrates natural gas conversion.

FIG. 12 illustrates syngas treatment.

FIG. 13 illustrates liquid fuels/chemicals production.

FIG. 14 illustrates Fischer-Tropsch production.

FIG. 15 illustrates hydrogen/oxygen production.

FIG. 16 illustrates an integrated superstructure.

FIG. 17 illustrates an overall process flowsheet diagram of the novel hybrid process.

FIG. 18 illustrates PFD 1: biomass and coal gassification trains (P100).

FIG. 19 illustrates PFD 2: syngas treatment units (P200).

FIG. 20 illustrates PFD 3: hydrocarbon generation section (P300).

FIG. 21 illustrates PFD 4: hydrocarbon upgrading section (P400).

FIG. 22 illustrates PFD 5: light gases reforming (continuation of P400).

FIG. 23 illustrates PFD 6: hydrogen and oxygen production, heat and power recovery section (P500 and P600).

FIG. 24 illustrates break-even oil price (BEOP) of seven process alternatives using distinct hydrogen prices. In each of panels C-R-A, C-E-A, B-R-A, B-E-A, H-R-A, H-E-A, and H-R-T, from left to right, the bars represent $2.50/kg H2, $2.00/kg H2, $1.50/kg H2 and $1.00/kg H2.

FIG. 25 illustrates break-even oil price (BEOP) using distinct electrolyzer capital costs and electricity prices. In each of panels C-E-A, B-E-A, and H-E-A, from left to right, the bars represent $0.08/kWh, $0.07/kWh, $0.06/kWh, $0.05/kWh, $0.04/kWh, and $0.03/kWh.

FIGS. 26A-B illustrate performance comparison of hydrogen-producing technologies (steam reforming of methane and electrolysis). FIG. 26A illustrates total fuel C vented and FIG. 26B illustrates BEOP. In each of panels H-R-A, H-E-A, and H-R-T, from left to right, the bars represent w/ Seq. and w/o Seq.

FIG. 27 illustrates a framework for the heat exchanger and power recovery network (HEPN). A simulated process flowsheet is analyzed to construct a list of (a) hot and cold streams, (b) hot and cold process units, (c) the process condensate, (d) the process cooling water requirement, and (f) the process electricity requirement. The hot and cold process units (list item b) are defined as all units that require heat or release heat at a given temperature. This process flowsheet information (list items a-f) is used along with a superset of heat engine operating conditions to sequentially determine (i) the minimum hot/cold/power utilities, (ii) the minimum number of heat exchanger matches, and (iii) the minimum annualized cost of heat exchange. The output from the HEPN is the optimal heat and power recovery network, which includes the total utility requirement, the operating conditions of the heat engines, and the topology of the heat exchanger network.

FIG. 28 illustrates a pictorial description of one heat engine with operating conditions (P_{b}^{B}, P_{c}^{C}, Tₜ).

FIG. 29 illustrates optimal HEPN topology for subnetwork 1 of the H-R-A flowsheet. All inlet and outlet temperatures given correspond to the actual stream temperatures of the match. Stream labels: H1, reverse water-gas-shift effluent; H6, fuel combuster effluent; H15, coal gasifier; H29, heat engine (75, 40, 900) precooler; C6, autothermal reactor (ATR) steam input; C7, ATR natural gas input; C8, ATR oxygen input; C9, ATR recycle light gas input; C33, heat engine (25, 1, 900) superheater; C34, heat engine (75, 40, 900) superheater; C35, heat engine (100, 15, 900) superheater. Heat engines are defined by the parameters P_{b}^{B} (bar), P_{c}^{C} (bar), and Tₜ (° C.).

FIG. 30 illustrates optimal HEPN topology for subnetwork 1 of the H-E-A flowsheet. All inlet and outlet temperatures given correspond to the actual stream temperature of the match. Stream labels: H1, reverse water-gas-shift effluent; H6, fuel combuster effluent; H12, coal gasifier; H27, heat engine (75, 40, 900) precooler; C6, autothermal reactor (ATR) steam input; C7, ATR natural gas input; C8, ATR oxygen input; C9, ATR recycle light gas input; C33, heat engine (25, 1, 900) superheater; C34, heat engine (25, 15, 500) superheater; C35, heat engine (75, 40, 900) superheater. Heat engines are defined by the parameters P_{b}^{B} (bar), P_{c}^{C} (bar), and Tₜ (° C.).

FIG. 31 illustrates optimal HEPN topology for subnetwork 1 of the H-R-T flowsheet. All inlet and outlet temperatures given correspond to the actual stream temperature of the match. Stream labels: H1, reverse water-gas-shift (RGS) effluent; H6, fuel combuster effluent; H17, coal gasifier; C1, RGS inlet hydrogen; C2, RGS recycle CO₂; C6, autothermal reactor (ATR) steam input; C7, ATR natural gas input; C8, ATR oxygen input; C9, ATR recycle light gas input; C33, heat engine (25, 1, 600) superheater; C34, heat engine (75, 1, 900) superheater; C35, heat engine (100, 15, 600) superheater. Heat engines are defined by the parameters P_{b}^{B} (bar), P_{c}^{C} (bar), and Tₜ (° C.).

FIG. 32 illustrates a Fischer-Tropsch (FT) hydrocarbon production flowsheet. Each of the six FT units has a distinct set of operating conditions including catalyst type (cobalt or iron), temperature (low—240° C., medium—267° C., and high—320° C.), and water-gas-shift reaction extent (forward, reverse, or none). Each unit is designed to produce either a minimal or nominal amount of wax (shown as a dashed line). The mathematical model will select at most two types of the six FT units to operate in a final process topology. All of the streams in FIG. 32 are variable.

FIG. 33 illustrates a First Fischer-Tropsch (FT) hydrocarbon upgrading flowsheet. The FT effluent may be passed through a series of stripper and flash units to separate the oxygenates and aqueous phase from the hydrocarbons. Alternatively, the effluent may be passed over a ZSM-5 catalytic reactor to convert most of the hydrocarbons into gasoline range species. The raw ZSM-5 product is then fractionated to remove any distillate or sour water from the gasoline product. All of the process streams in FIG. 33 are variable.

FIG. 34 illustrates a second Fischer-Tropsch (FT) hydrocarbon upgrading flowsheet. The water lean FT effluent is fractionated and passed through a series of treatment units to recover the gasoline, diesel, and kerosene products along with some LPG byproduct. Light gases (i.e., unreacted syngas and C₁-C₂ hydrocarbons) are collected and recycled back to the process.

FIG. 35 illustrates a methanol synthesis and conversion flowsheet. Clean syngas is initially converted to methanol and then split to either the methanol to gasoline (MTG) or methanol to olefins (MTO) processes. The two processes utilize a ZSM-5 zeolite to convert the methanol to either gasoline range hydrocarbons (MTG) or olefins which are subsequently oligomerized to gasoline and distillate range hydrocarbons (MOGD). The distillate is hydrotreated to form diesel or kerosene which the gasoline range hydrocarbons are sent to an LPG-gasoline separation system. All of the streams in FIG. 35 are variable.

FIG. 36 illustrates an LPG-gasoline product separation flowsheet. The raw HC products from the FT-ZSM5 unit, the MTG unit, or the MOGD process are passed through a series of separation units to recover a gasoline product and an LPG byproduct. Light gases are recycled back to the refinery and CO₂ recovery may be utilized in preparation for sequestration or reaction with H₂ via the reverse water-gas-shift reaction. All of the streams in FIG. 36 are variable.

FIG. 37 illustrates a parametric analysis of feedstock cost. The histogram shows the number of counts (out of 27) for break-even oil price (BEOP) when low, nominal, and high values are used for the costs of coal, biomass, and natural gas.

FIG. 38 illustrates a biomass gasification process flowsheet.

FIG. 39 illustrates a coal gassification process flowsheet.

FIG. 40 illustrates a syngas cleaning process flowsheet.

FIG. 41 illustrates a claus sulfur recovery process flowsheet.

FIG. 42 illustrates a Fischer-Tropsch hydrocarbon production process flowsheet. All of the streams in FIG. 42 are variable.

FIG. 43 illustrates a first Fischer-Tropsch hydrocarbon upgrading process flowsheet. All of the streams in FIG. 43 are variable.

FIG. 44 illustrates a second Fischer-Tropsch hydrocarbon upgrading process flowsheet.

FIG. 45 illustrates a methanol synthesis and conversion process flowsheet. All of the streams in FIG. 45 are variable.

FIG. 46 illustrates an LPG-gasoline separation process flowsheet. All the streams in FIG. 46 are variable.

FIG. 47 illustrates a recycle gas treatment process flowsheet.

FIG. 48 illustrates a hydrogen/oxygen production process flowsheet.

FIG. 49 illustrates a process wastewater treatment process flowsheet.

FIG. 50 illustrates a utility cycle wastewater treatment process flowsheet.

FIG. 51 illustrates a natural gas conversion flow sheet. Natural gas is combined with recycle methane and may be converted to (1) synthesis gas (CO, CO₂, H₂, and H₂O) via steam reforming or ATR, (2) methanol using catalytic partial oxidation, or (3) olefins (ethylene/propylene) via OC.

FIG. 52 illustrates a flow sheet of natural gas utilities. Natural gas and recycle fuel gas may be utilized to produce electricity through a GT or additional process heat via a fuel combustor. The effluent from both of these processes are cooled and then are either vented or passed over a CO₂ recovery unit to capture and process the produced CO₂.

FIG. 53 illustrates a Synthesis gas (syngas) handling flow sheet. Syngas may be passed over a forward/reverse WGS reactor to alter the H₂ to CO/CO₂ ratio prior to FT or methanol synthesis. The syngas is then cooled, flashed to remove water, and may be directed to a one-stage Rectisol unit for CO₂ removal. The captured CO₂ may be vented, sequestered, or recycled back to process units.

FIG. 54 illustrates a PFD for case study U-1.

FIG. 55 illustrates a PFD for case study K-50.

FIG. 56 illustrates a parametric analysis of natural gas cost. The BEOP is plotted for the case studies with an unrestricted product composition as a function of the natural gas price in TSCF.

FIG. 57 illustrates a natural gas conversion process flowsheet.

FIG. 58 illustrates a natural gas utility process flowsheet.

FIG. 59 illustrates a synthesis gas handling process flowsheet.

FIG. 60 illustrates a Fischer-Tropsch hydrocarbon production process flowsheet. All of the streams in FIG. 60 are variable.

FIG. 61 illustrates a first Fischer-Tropsch hydrocarbon upgrading process flowsheet. All of the streams in FIG. 61 are variable.

FIG. 62 illustrates a second Fischer-Tropsch hydrocarbon upgrading process flowsheet.

FIG. 63 illustrates a methanol synthesis and conversion process flowsheet. All of the streams in FIG. 63 are variable.

FIG. 64 illustrates an LPG-gasoline separation process flowsheet. All of the streams in FIG. 64 are variable.

FIG. 65 illustrates a hydrogen/oxygen production process flowsheet.

FIG. 66 illustrates a process wastewater treatment process flowsheet.

FIG. 67 illustrates a utility cycle wastewater treatment process flowsheet.

FIGS. 68A-68D illustrate branch-and-bound progression for the small case studies. At each node in the branch-and-bound tree, the current lower (lower line) and upper bounds (upper line) (in $/GJ) are shown along with the optimality gap (dotted line) for feedstock-carbon conversion rates of (a) 25% in FIG. 68A, (b) 50% in FIG. 68B, (c) 75% in FIG. 68C, and (d) 95% in FIG. 68D. The upper

FIGS. 69A-69D illustrate branch-and-bound progression for the medium case studies. At each node in the branch-and-bound tree, the current lower (lower line) and upper bounds (upper line) (in $/GJ) are shown along with the optimality gap (dotted line) for feedstock-carbon conversion rates of 25% in FIG. 69A, 50% in FIG. 69B, 75% in FIG. 69C, and 95% in FIG. 69D.

FIGS. 70A-70D illustrate branch-branch-and-bound progression for the large case studies. At each node in the branch-and-bound tree, the current lower (lower line) and upper bounds (upper line) (in $/GJ) are shown along with the optimality gap (dotted line) for feedstock-carbon conversion rates of 25% in FIG. 70A, 50% in FIG. 70B, 75% in FIG. 70C, and 95% in FIG. 70D.

FIG. 71 illustrates a first wastewater treatment flowsheet. Sour product upgrading wastewater from the wax hydrocracker (WHC), the hydrocarbon recovery unit (HRC), distillate hydrotreater (DHT), and naphtha hydrotreater (NHT) are mixed (MXPUWW) and split (SPPUWW) to either the biological digestor (BD) or the sour stripper (SS). Post-combustion knockout from the fuel combustor flash (FCF) and the gas turbine flash (GTF) are mixed (MXPCKO) and split (SPPCKO) to the (SS) unit, the (BD) unit, or to the outlet wastewater mixer (MXWW). Acid rich wastewater from the Fischer-Tropsch upgrading units (MXFTWW), the acid gas flash (AGF), and the Claus flash (CF) is mixed (MXSS) and sent to the (SS) unit. Output from the (BD) unit is split (SPBD) and output (MXWW) or sent to the electrolyzer (MXEYZ), the deaerator (MXDEA), or the cooling tower (MXCLTR). The output from the (SS) unit is split (SPSS) and sent to the (BD) unit or to the outlet. Sour gas from the (SS) unit is compressed (SGC) and recycled to the process while the biogas from the (BD) unit is sent to the Claus combustor (CC). All fixed process units are represented by 110, variable process units are represented by 120, variable process streams are represented by 210 and all other process streams are fixed unless otherwise indicated. Splitters are represented by 130 and mixers are represented by 140.

FIG. 72 illustrates a second wastewater treatment process flowsheet. The blowdown from the cooling tower (CLTR) is split (SPCLTR) and either recycled back to the tower (MXCLTR) or sent to the reverse osmosis mixer (MXRO), the deaerator mixer (MXDEA), or the outlet wastewater mixer (MXWW). The water leaving the (MXDEA) unit is fed to the deaerator (DEA) before being split (SPDEA) to the heat and power system (HEP) or generate steam through the process water boiler (XPWB). The blowdown from the (HEP) and the (XPWB) is mixed (MXBLR) and split (SPBLR) to either the (MXDEA) unit, the (MXRO) unit, the (MXCLTR) unit, or the (MXWW) unit. Steam generated from the XPWB unit is split (SPSTM) and fed to either the biomass gasifiers (BGS and BRGS), the coal gasifiers (CGS and CRGS), the auto-thermal reactor (ATR), or the water-gas-shift reactor (WGS). All solid waste from the reverse osmosis (RO) unit is dumped from the process while the treated water is split (SPRO) and recycled to various process units. Inlet freshwater is split (SPH2O) and sent to water treatment units or to the electrolyzer mixer (MXEYZ). All fixed process units are represented by 110, variable process units are illustrated by 120, variable process streams are represented by 210, and all other process streams are fixed process streams unless otherwise indicated. For clarity, the variable streams leaving the cooling tower are shown as dashed lines. Splitters are represented by 130 and mixers are represented by 140. The working fluid for the heat engines is represented by 310 and the process cooling water is represented by 410.

### DETAILED DESCRIPTION OF THE PREFERRED EMBODIMENTS

Certain terminology is used in the following description for convenience only and is not limiting. The words “right,” “left,” “top,” and “bottom” designate directions in the drawings to which reference is made. The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

Incorporating biomass in fuel production can help reduce GHG emissions due to the carbon uptake from the atmosphere during biomass growth and cultivation, although its amount is limited by the available land area for biomass. Hybrid processes utilizing coal, biomass, and natural gas can take advantage of the benefits of each raw material to yield processes that can be economically competitive with petroleum-based fuels and have reduced GHG emissions.

A novel hybrid energy process was developed that utilizes coal, biomass, and natural gas as feedstocks to produce any given volumetric capacity of liquid fuels or chemicals, e.g., gasoline, diesel, kerosene. The process will produce syngas from each of the three feedstocks and subsequently convert that syngas to liquid fuels via the Fischer-Tropsch reaction or through a methanol intermediate. The raw hydrocarbons from the Fischer-Tropsch reaction can be converted to the desired liquid fuels via (a) distillation and additional upgrading (e.g., hydrocracking, hydrotreating, isomerization) or (b) catalytic conversion over a ZSM-5 zeolite. The intermediate methanol can be upgraded to the desired liquid fuels using (a) direct conversion over a ZSM-5 zeolite or (b) conversion to olefins followed by conversion of the olefins over a ZSM-5 zeolite.

The mixture of feedstocks may mitigate the risk involved with price and demand uncertainty that may be associated with a single feedstock refinery, and the combination of feedstocks allows the process to draw on key advantages of each feedstock that would not be otherwise obtainable. The low cost of coal, the greenhouse gas reduction potential of biomass, and the high hydrogen content of natural gas may combine to help design the most economically robust refinery possible. The refinery may be capable of converting any fraction of input carbon in the coal, biomass, and natural gas to liquid fuels by recycling CO₂ in a closed-loop system using the reverse water-gas-shift reaction. Through the use of biomass feedstock, a CO₂ recycle loop, and CO₂ sequestration, the refinery can be readily designed to have a very small or net negative amount of total greenhouse gas emissions for each gallon of product produced.

Using innovative combinations of unit operations not found in other process designs, a superstructure detailing a wide array of process topologies is postulated and a mixed-integer nonlinear optimization model was developed to examine the economic trade-offs between each topology and choose the solution with the best economic value. The model for process synthesis was enhanced by simultaneously including both the costs and emissions associated with utility generation via gas turbines, steam turbines, and a detailed heat exchanger. Additionally, the refinery integrates a comprehensive wastewater network which utilizes a superstructure approach to determine the appropriate series of process units that are needed to minimize wastewater contaminants and freshwater intake. The detailed topological superstructure of the proposed refinery provides definite advantages over current technologies that utilize a specific set of process units because the current invention may be capable of finding a more efficient design methodology.

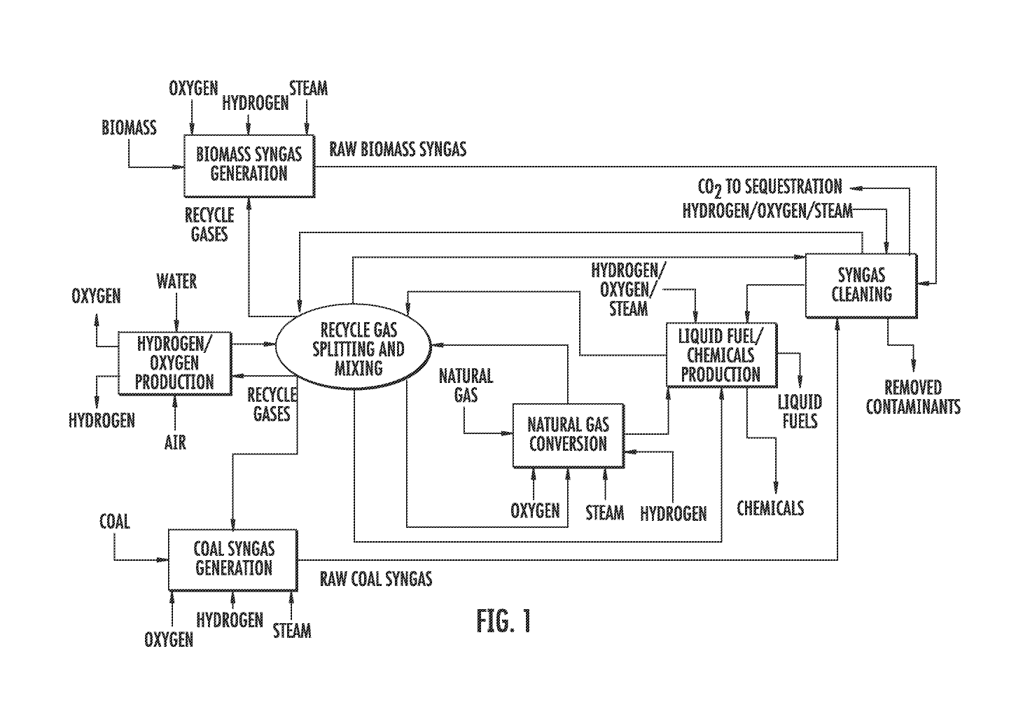

Referring to FIG. 1, a new process to convert coal, biomass, or natural gas feedstocks to synthetic liquid hydrocarbons is shown. The proposed process can address all combinations of one, two, or three of these feedstocks. The process initially consists of up to three sections that are dedicated to producing synthesis gas from coal, biomass, or natural gas, respectively. The technologies involved with coal or biomass synthesis gas generation may include gasification or pyrolysis based systems which may utilize oxygen or steam to produce the gas. Recycle gases may be directed to either of these two sections for generation of additional synthesis gas.

The process may be a composition of unit operations designed to convert coal, biomass, and natural gas to gasoline, diesel, or kerosene. This process involves seven distinct stages including (i) biomass synthesis gas generation, (ii) coal synthesis gas generation, (iii) natural gas conversion, (iv) synthesis gas cleanup, (v) liquid fuels production, (vi) recycle gas handling, and (vii) hydrogen/oxygen production. This is shown as a topological superstructure in FIG. 1.

Embodiments include a process flowsheet that utilizes coal, biomass, natural gas, or any combination of those three and converts them to liquid fuels or chemicals via (i) a synthesis gas intermediate, (ii) a methanol intermediate, and (iii) an ethylene intermediate. What is shown in FIG. 1 represents a superstructure of all possible alternatives for an embodiment of process design. A superstructure is defined to mean a combination of all possible unit operations and streams that can convert any or all of the three feedstocks to liquid fuels or chemicals. All subsets of the superstructure shown in FIG. 1 are embodiments herein. Individual embodiments include each process design that is part of the superstructure, even if the covered designs may not contain all of the units or streams that are present in the flowsheet. All of the arrows shown in FIG. 1 may correspond to one or multiple streams that are passed to/from each section of the refinery. The arrows in the figure are used to convey that material from one section of the plant may be transferred to another section of the plant, though this transfer may be accomplished through the use of one or more streams.

Synthesis gas is produced from gasification of the coal and biomass using distinct, parallel biomass and coal gasification trains in sections (i) and (ii), respectively. The biomass and coal gasifiers can either operate with only a solid feedstock input or in tandem with additional vapor phase fuel inputs from elsewhere in the refinery. The natural gas feedstock enters downstream of the Fischer-Tropsch units in section (iii) and is converted to synthesis gas in an auto-thermal reactor, directly converted to methanol, or directly converted to ethylene.

The syngas from the gasifier trains is sent to the gas cleanup area in section (iv) where a reverse water-gas-shift unit may be used to alter the ratio of H₂ to CO in the feed. Other units in section (iv) are designed to remove acid gases from the synthesis gas stream and separate out H₂O and CO₂ if necessary. CO₂ may be recycled to other process units in the refinery or compressed for sequestration. Once cleaned of all necessary acid gases, the synthesis gas is sent to section (v) for production of raw hydrocarbons via a Fischer-Tropsch reaction or a methanol synthesis. One or multiple of six total Fischer-Tropsch reactors can be utilized to produce a raw hydrocarbon composition that will be upgraded to liquid product. Methanol may also be produced from the synthesis gas to be sold as a byproduct or converted to liquid fuels.

The raw Fischer-Tropsch hydrocarbons and the methanol are then upgraded to final hydrocarbon products. The Fischer-Tropsch hydrocarbons may be converted to gasoline via a ZSM-5 catalyst or may be fractionated using a distillation column and upgraded to gasoline, diesel, and kerosene using a combination of hydrocrackers, hydrotreaters, isomerizers, reformers, alkylation units, and additional distillation columns. The methanol may be converted to gasoline via a ZSM-5 catalyst or converted to diesel and kerosene via an intermediate conversion to olefins.

Recycle gases generated from various units throughout the refinery may be sent to sections (i) and (ii) to feed the gasifiers, to section (iii) for reforming, to section (iv) for CO₂ removal, to section (v) for hydrocarbon synthesis, or section (vii) for hydrogen production. The hydrogen in the refinery can be produced through a pressure-swing adsorption unit or via an electrolyzer unit in section (vii). Hydrocarbon-rich light gases may be fed to the pressure-swing adsorption unit to produce a near-100% hydrocarbon stream while the electrolyzer may input freshwater or recycle process water. The oxygen for the system can be provided by the electrolyzer unit or a separate air separation unit which may be utilized to produce a high-purity oxygen stream.

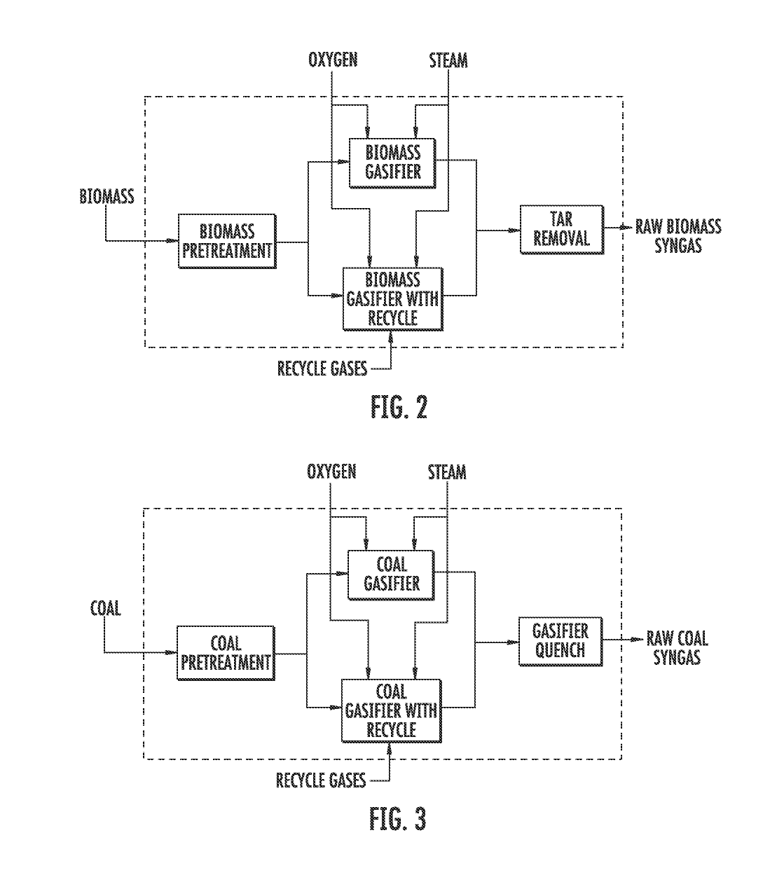

Referring to FIGS. 2 and 3, examples of coal and biomass synthesis gas generation using gasification technology are illustrated, respectively. The technologies involved with natural gas conversion include, but are not limited to, auto-thermal reforming, partial oxidation, steam reforming, direct conversion to methanol, and direct conversion to ethylene. Recycle gases may be directed to this section for generation of additional synthesis gas.

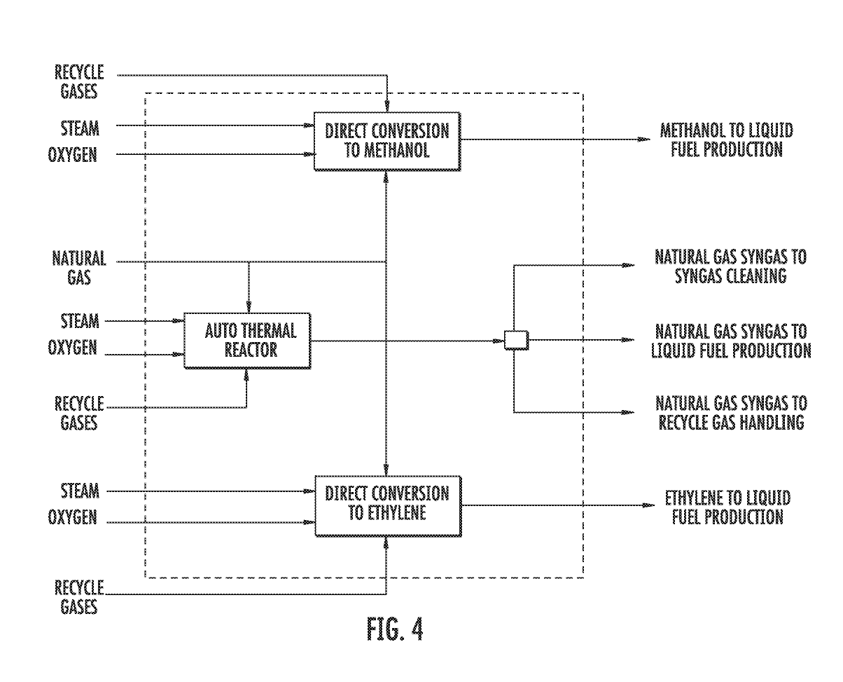

Referring to FIG. 4, an example of natural gas synthesis gas generation using auto-thermal reforming technology is illustrated.

The synthesis gas generated from biomass or coal sources may be initially cleaned to remove any acid gases that may poison catalysts during liquid fuel production. The natural gas entering the synthesis gas generation section may already be stripped of acidic gases, so the effluent synthesis gas may be directed either to the syngas cleaning section, the liquid fuel production section, or it may be recycled back to the process. All acid gases will be removed from the system in the syngas cleaning section and CO₂ may be captured and either compressed for sequestration or recycled back to the process.

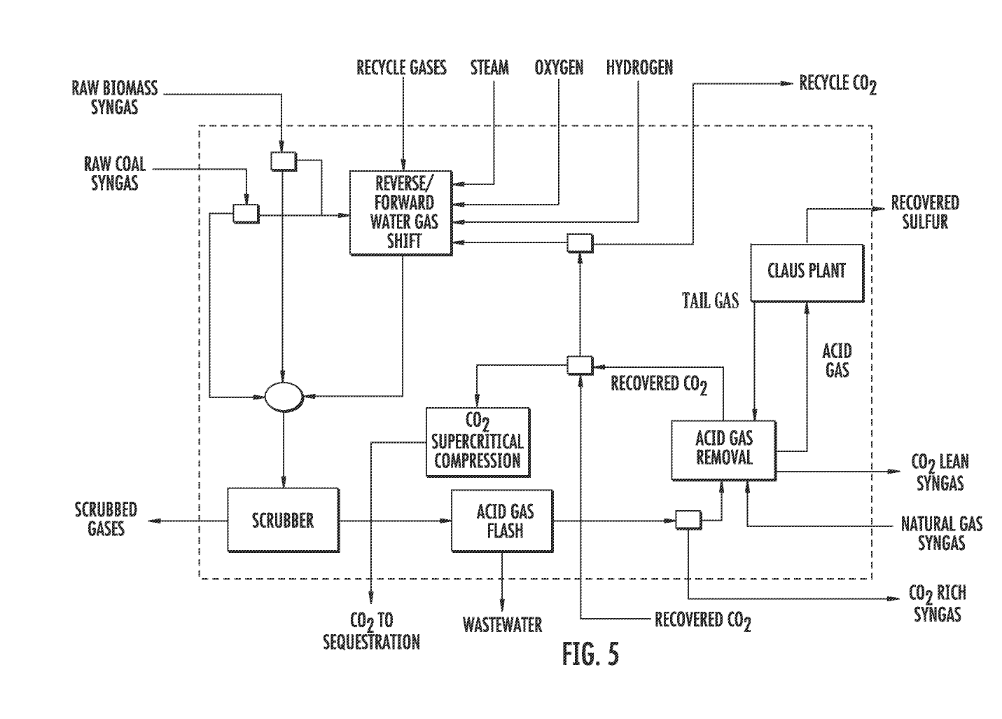

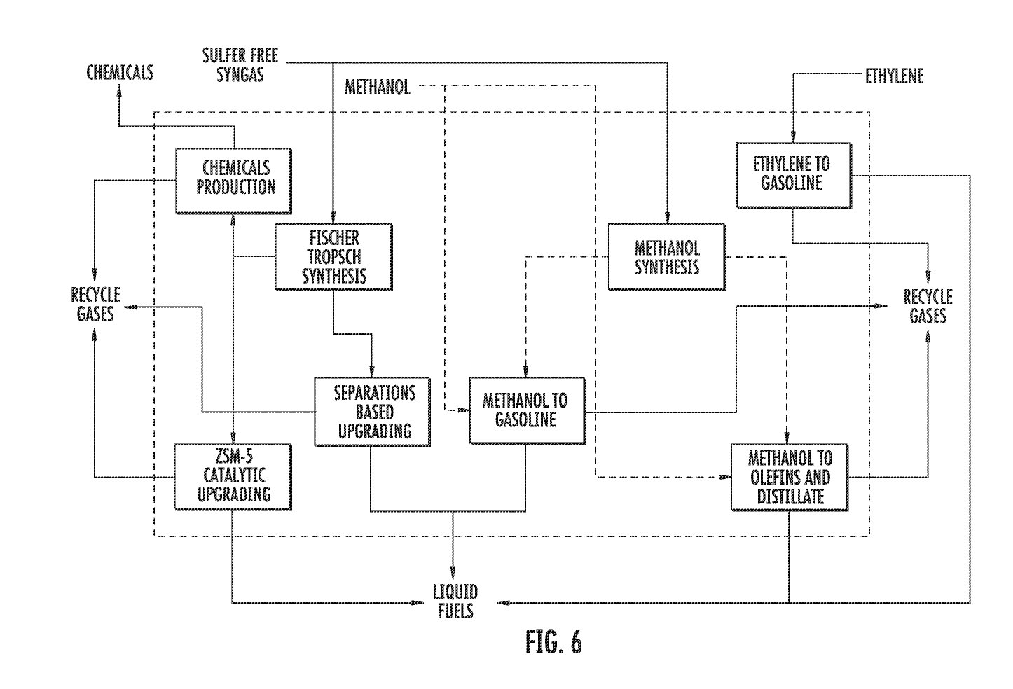

Referring to FIG. 5, an example of a synthesis gas cleaning section is illustrated. The raw biomass and coal synthesis gas is partially split to a water-gas-shift unit where either (i) the forward water-gas-shift reaction is encouraged to increase the H₂/CO ratio of the gas or (ii) the reverse water-gas-shift reaction is encouraged to reduce the concentration of CO₂. Acid gases are removed via scrubbing, wastewater removal, sulfur removal, or CO₂ removal. Sulfur free syngas (either CO₂ lean or CO₂ rich) is directed to liquid fuels production.

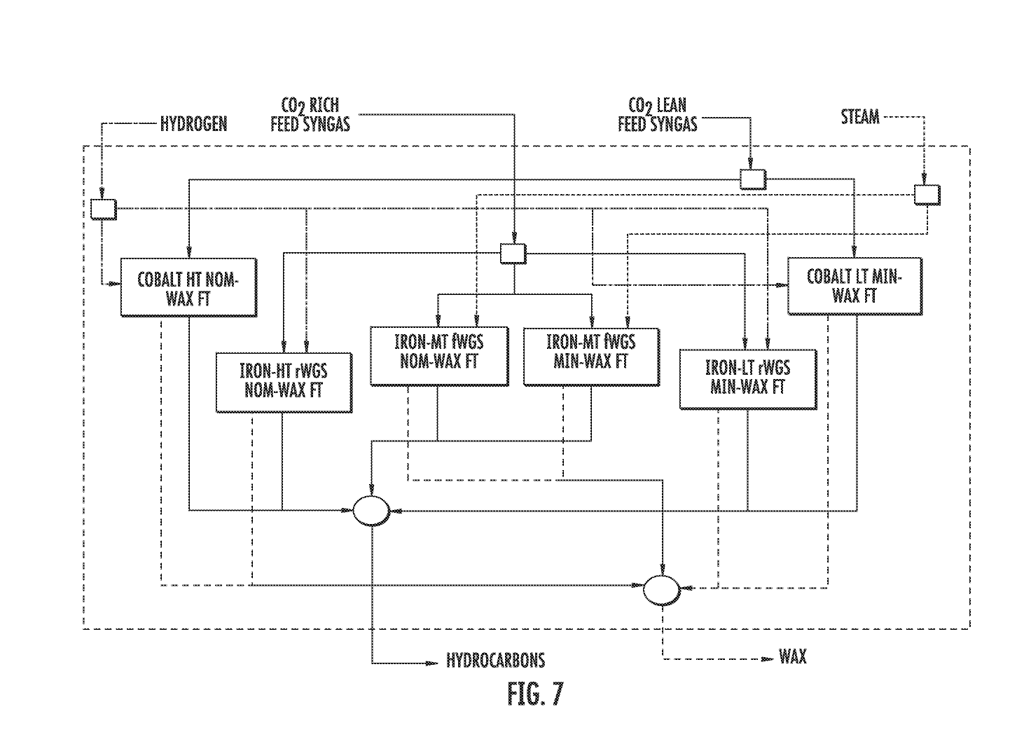

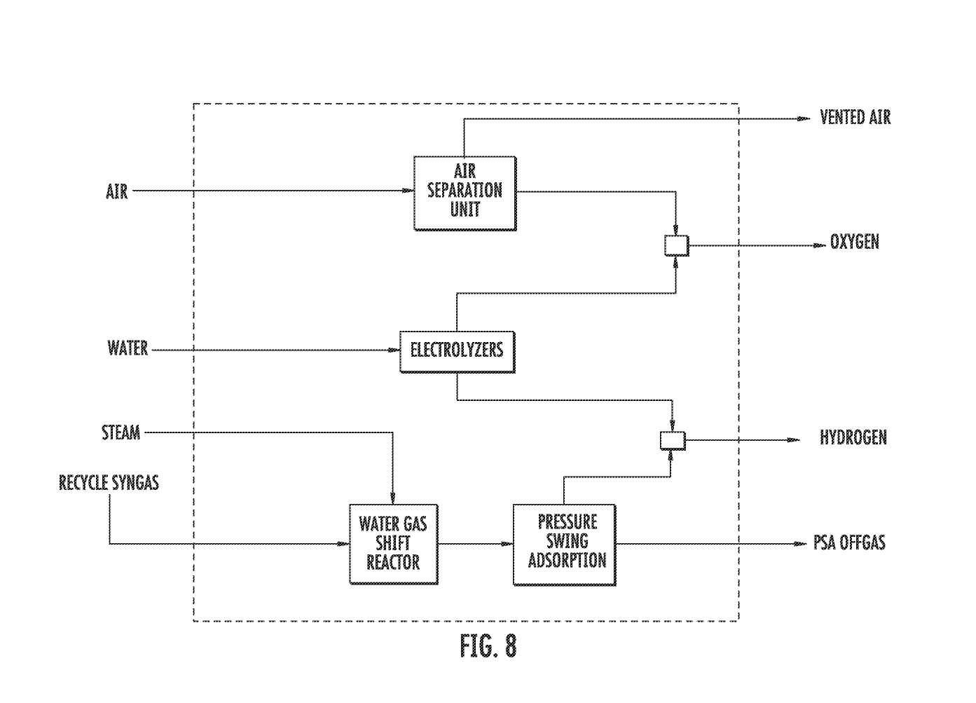

The sulfur free synthesis gas is converted to a liquid stream via the Fischer-Tropsch synthesis or methanol synthesis in the liquid fuels production section. Referring to FIG. 6, an example of this section is shown. Referring to FIG. 7, a detailed example of a Fischer-Tropsch synthesis section is shown. The product from the Fischer-Tropsch synthesis section may be directed to either a separations based upgrading or a ZSM-5 catalytic upgrading section while the methanol may either be converted to gasoline or to a distillate via conversion over a ZSM-5 catalyst or conversion to olefins followed by subsequent conversion over the ZSM-5 catalyst, respectively. Examples of typical hydrocarbons are liquid fuels such as gasoline, diesel, or kerosene. Embodiments herein are an improvement on current refineries based on (i) the capability to produce synthesis gas from coal, biomass, or natural gas, (ii) the capability to produce any combination of gasoline, diesel, or kerosene fuels, (iii) the use of one or multiple technologies to convert the synthesis gas to the final liquid product.

Examples of technologies present in part (iii) include six Fischer-Tropsch reactors operating at three different temperatures and using either cobalt or iron catalyst, the capability to upgrade the raw hydrocarbons produced in the six Fischer-Tropsch reactors using a ZSM-5 catalyst or a series of treatment units including a hydrocracker, a reformer, hydrotreaters, isomerizers, and an alkylation unit, a methanol synthesis reactor to produce methanol for sale as a byproduct or use as an intermediate, a methanol to gasoline reactor to convert intermediate methanol to gasoline, and a methanol to olefins and diesel/kerosene reactor to convert intermediate methanol to diesel and kerosene.

Referring to FIG. 8, hydrogen and oxygen production for the refinery is shown. The hydrogen in the refinery can be produced through pressure-swing adsorption or via electrolysis of water. Hydrocarbon-rich light gases will be fed to the pressure-swing adsorption unit to produce a near-100% hydrocarbon stream while the electrolyzer may input freshwater or recycle process water. The oxygen for the system can be provided by the electrolyzer unit or a separate air separation unit which may be utilized to produce a high-purity oxygen stream.

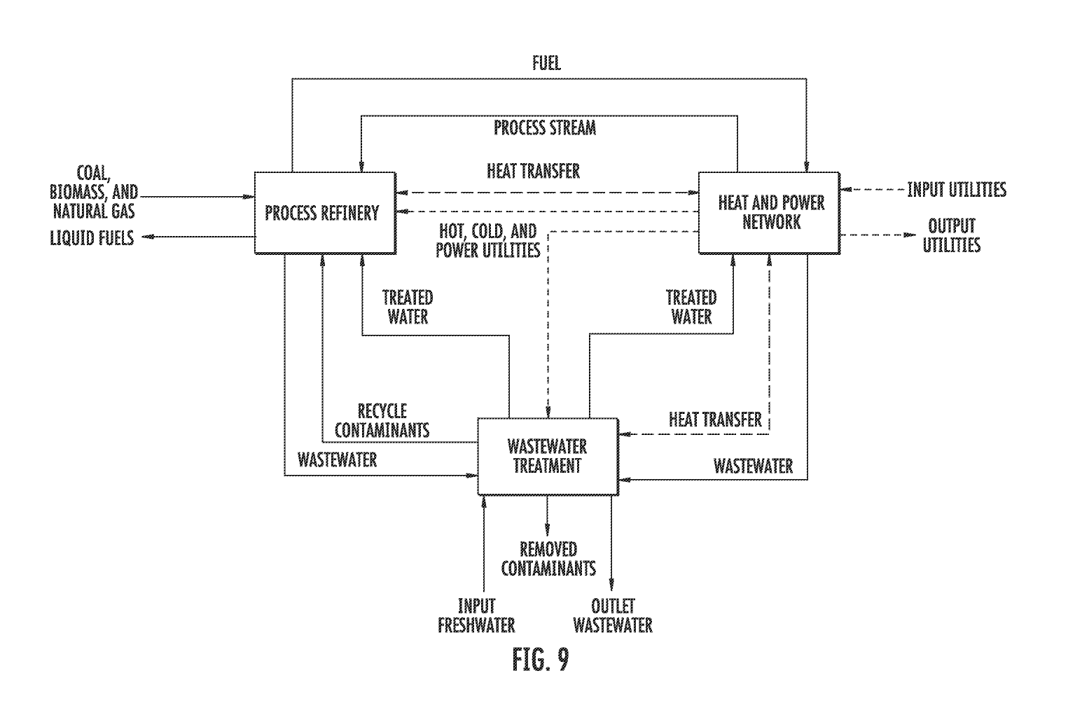

Referring to FIG. 9, in addition to the set of unit operations detailed above for the process refinery, the process may also contain a combined heat, power, and water integration as illustrated. Heat may be transferred from the process refinery and a wastewater treatment section via a heat and power network which may be used to generate hot, cold, and power utilities needed for the process refinery and wastewater treatment. Fuel gas may also be provided from the process refinery for utility generation and may include natural gas or recycle synthesis gas. Excess utilities may be output from the process and sold as a byproduct and utilities may also be purchased if necessary. Wastewater produced from the process refinery and the heat and power network is directed to the wastewater treatment section where contaminants may be removed from the water and either recycled back to the refinery or removed from the system. Treated water is sent to the process refinery or to the heat and power network. Any steam needed for the process refinery may be generated from the heat and power network.

The process may be used to help satisfy the national demand for liquid transportation fuels using a variety of domestically available types of coal, biomass, and natural gas. The process has immediate application in key areas throughout the nation where coal, biomass, or natural gas feedstocks are abundant and have a low purchase and delivery cost. However, the process can be used at any location to produce a desired quantity of liquid fuels. The applicability of embodiments herein may increase in the future with (i) increasing cost of crude oil, (ii) the implementation of a carbon tax on liquid fuel production, (iii) enhanced government initiatives to produce liquid fuels from alternative sources, (iv) increasing feedstock availability, (v) decreasing feedstock cost, and (vi) decreasing investment cost of unit operations.

The process includes but is not limited to having the following features or benefits: (i) the ability to use a combination of coal, biomass, and natural gas feedstocks to produce synthesis gas, (ii) the utilization of coal and biomass gasifiers that can be fed either with solid feedstocks or a combination of solid and vapor feeds, (iii) a reverse water-gas-shift reactor to consume CO₂ using produced hydrogen, (iv) recycle of CO₂ throughout the process to consume additional CO₂ within various process units, (v) a combination of six Fischer-Tropsch units using multiple temperature levels and either iron or cobalt catalysts to produce different hydrocarbon effluent compositions, (vi) a combination of a ZSM-5 catalyst or a series of hydrocracker, hydrotreater, isomerizer, and alkylation units to produce gasoline, diesel, and kerosene, (vii) a methanol synthesis reactor to produce byproduct or intermediate methanol, (viii) a combination of methanol to gasoline or methanol to diesel and kerosene units to produce the liquid fuels, (ix) a hydrogen/oxygen production system including an air separation unit, a pressure-swing adsorption unit, and electrolyzer units that is capable of producing hydrogen and oxygen from both carbon and non-carbon based sources, and (x) a utility plant that will produce electricity and process heat using a gas turbine, a steam turbine, and a series of heat exchangers.

The process offers at least the following advantages. First, embodiments may contain a mixture of at least one of coal, biomass, and natural gas feedstocks which will inherently mitigate the risk involved with price and demand uncertainty that may be associated with a single feedstock refinery. Additionally, the combination of feedstocks allows the invention to draw on key advantages of each feedstock that would not be otherwise obtainable. The low cost of coal, the greenhouse gas reduction potential of biomass, and the high hydrogen content of natural gas may combine to design the most efficient and economic refinery possible. Second, the process may have the capability to convert any fraction of the input carbon in the coal, biomass, and natural gas to liquid fuels. Embodiments may be capable of directly analyzing economic tradeoffs between using feedstock produce either liquid fuels or byproduct electricity when given a minimum threshold of carbon conversion. Third, the process may be capable of producing liquid fuels using a variety of process technologies. Current processes utilize only a small number of these technologies within the plant design and may ultimately lead to inefficient process designs. The current process may produce a more efficient design based on the inclusion of additional process considerations.

The limitations of the proposed framework are based upon the exclusion of certain topologies from consideration in the overall design. These limitations are overcome by extending the refinery design alternatives to include specific process units that will fulfill the desired goal that is not met by the current invention. Examples of these limitations include but are not limited to (i) the ability to produce only a select group of synthetic hydrocarbons based upon the outputs of the Fischer-Tropsch reactor or the methanol synthesis reactor, (ii) the use of only thermochemical based production of liquid hydrocarbons as opposed to biological or catalytic based production, and (iii) the use of only indirect liquefaction of feedstocks as opposed direct liquefaction of feedstocks.

Described herein are novel GTL processes that can convert natural gas to produce any given volumetric capacity of gasoline, diesel, and kerosene. Natural gas may be directly converted to higher hydrocarbons or to an intermediate (e.g., synthesis gas, methanol) which may be subsequently converted to hydrocarbon species. The synthesis gas may be converted to raw hydrocarbons via the Fischer-Tropsch reaction or through a methanol intermediate. Hydrocarbons from the process can be converted to the desired liquid fuels via (a) distillation and additional upgrading (e.g., hydrocracking, hydrotreating, isomerization) or (b) catalytic conversion over a ZSM-5 zeolite. The intermediate methanol may be upgraded to the desired liquid fuels using (a) direct conversion over a ZSM-5 zeolite or (b) conversion to olefins followed by conversion of the olefins over a ZSM-5 zeolite. Lifecycle GHG emissions for the GTL processes may be reduced via CO₂ capture and sequestration in geological formations (e.g., saline aquifers) or capture and recycle of the CO₂ to the process for consumption via the reverse water-gas-shift reaction. The latter method is an important means of reducing the lifecycle emissions while simultaneously increasing the overall carbon yield of the liquid fuels.

Using innovative combinations of unit operations not found in other process designs, a superstructure detailing a wide array of process topologies is provided and a mixed-integer nonlinear optimization model was developed to examine the economic trade-offs between each topology and chose the solution with the best economic value. The model for process synthesis was enhanced by simultaneously including both the costs and emissions associated with utility generation via gas turbines, steam turbines, and a detailed heat exchanger. Additionally, the refinery integrates a comprehensive wastewater network which utilizes a superstructure approach to determine the appropriate series of process units that are needed to minimize wastewater contaminants and freshwater intake. The detailed topological superstructure of the proposed refinery provides definite advantages over current technologies that utilize a specific set of process units because it may always be capable of finding a more efficient design methodology.

The processes are economically competitive with petroleum-based fuels with a level of GHG emissions equivalent to the well-to-wheel emissions for a standard petroleum refinery. For processes with capacities between 10,000 barrels per day (BPD)—200,000 BPD that utilize natural gas at a price of $5/thousand standard cubic foot (TSCF), the liquid fuels produced will be economically superior when crude oil is priced above $50-$70 per barrel. Optimal placement of the refinery in specific locations with lower costs of natural gas can significantly improve the potential profit achieved from the refinery. For example, natural gas costing $3/TSCF will make a 10,000 BDP refinery competitive when crude oil is above $45-$50 per barrel and a 1,000 BPD refinery competitive at $80-$90 per barrel.

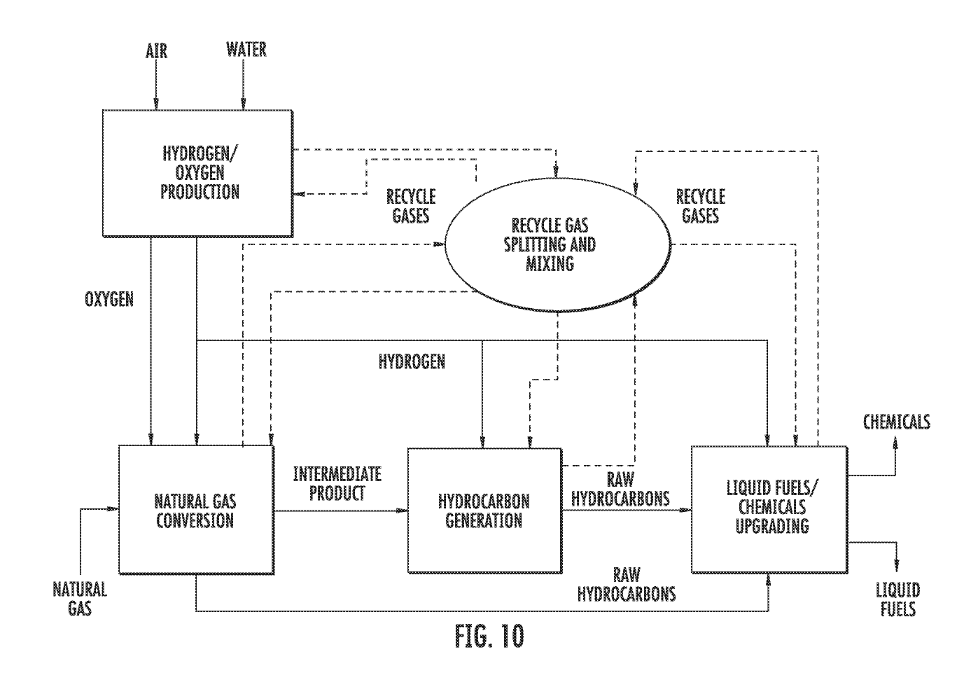

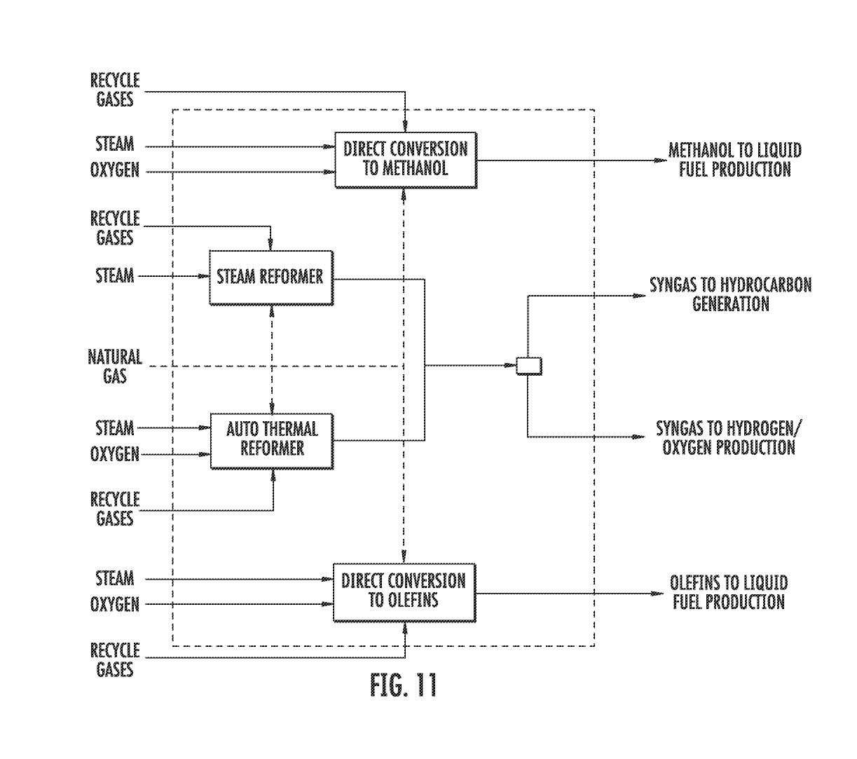

Described herein are process refineries that can convert a natural gas feedstock to synthetic liquid hydrocarbons (FIG. 10). The refineries consist of up to six major sections that specifically focus on (a) removal of natural gas liquids and sulfur to form a methane-rich natural gas, (b) natural gas conversion to hydrocarbons or other intermediate materials (e.g., synthesis gas, methanol, chlorinated hydrocarbons, etc.), (c) conversion of intermediate materials to hydrocarbons, (d) upgrading of the hydrocarbons to the final liquid product (e.g., gasoline, diesel, kerosene), (e) processing of recycle gases, and (f) hydrogen/oxygen production. The proposed process consists of two major components: (1) a process synthesis model that is capable of identifying economically and environmentally superior natural gas to liquids refineries when given a set of candidate technologies and (2) new process refineries that have been developed through the model described in (1).

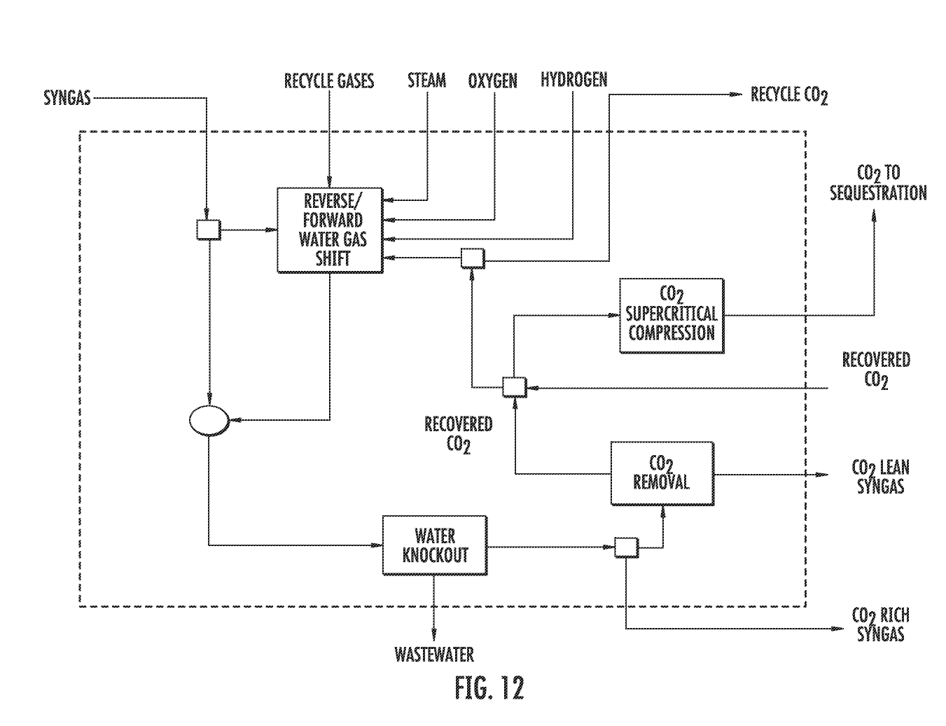

The technologies involved with natural gas conversion include auto-thermal reforming, steam reforming, partial oxidation to methanol, and oxidative coupling to olefins. Recycle gases may be directed to this section for generation of additional natural gas conversion products. An example of natural gas synthesis gas generation using four distinct technologies is present in FIG. 11. The process synthesis model is capable of analyzing additional natural gas conversion technologies which include, but are not limited to, compact reforming, carbon dioxide reforming, and oxygen membrane reforming. Auto-thermal reforming or steam reforming of the natural gas may generate synthesis gas (e.g., CO, H₂, CO₂, H₂O) that can be converted to liquid hydrocarbons. The methane-rich natural gas may already be stripped of sulfur species (e.g., H₂S), so effluent synthesis gas may not require additional sulfur removal. The synthesis gas is partially split to a water-gas-shift unit where either (i) the forward water-gas-shift reaction is encouraged to increase the H₂/CO ratio of the gas or (ii) the reverse water-gas-shift reaction is encouraged to reduce the concentration of CO₂. CO₂ may also be captured and either compressed for sequestration, recycled back to the process, or vented to the atmosphere. An example of a synthesis gas treatment section is shown in FIG. 12 and is considered to be part of the natural gas conversion section shown in FIG. 10.

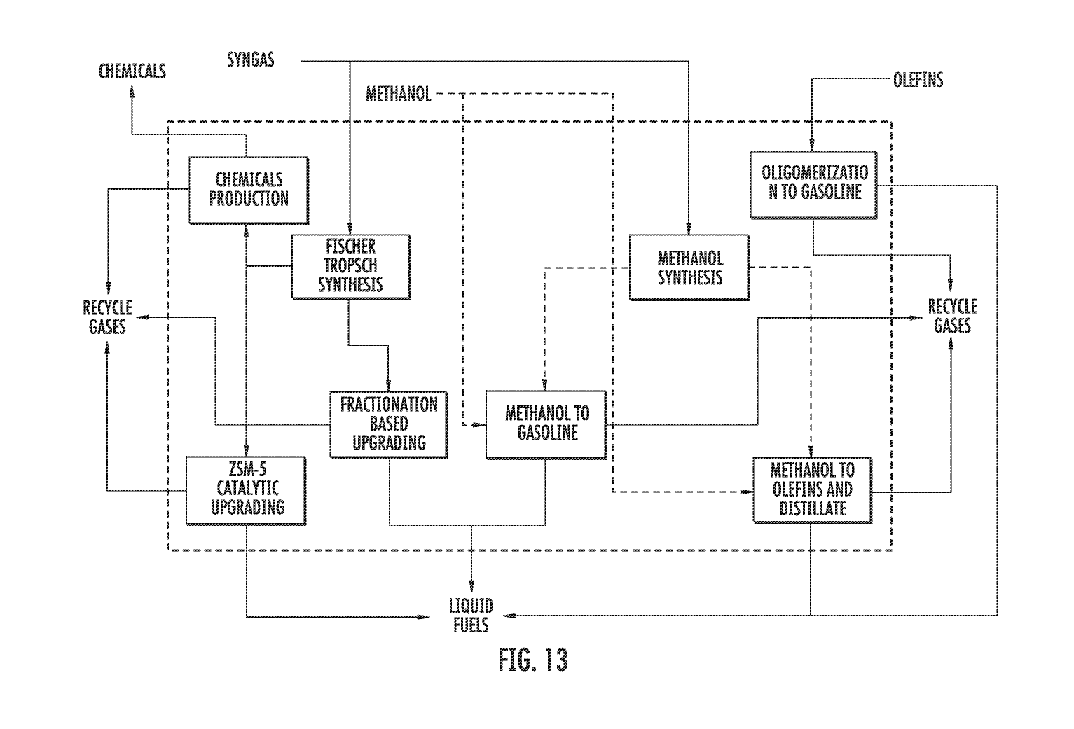

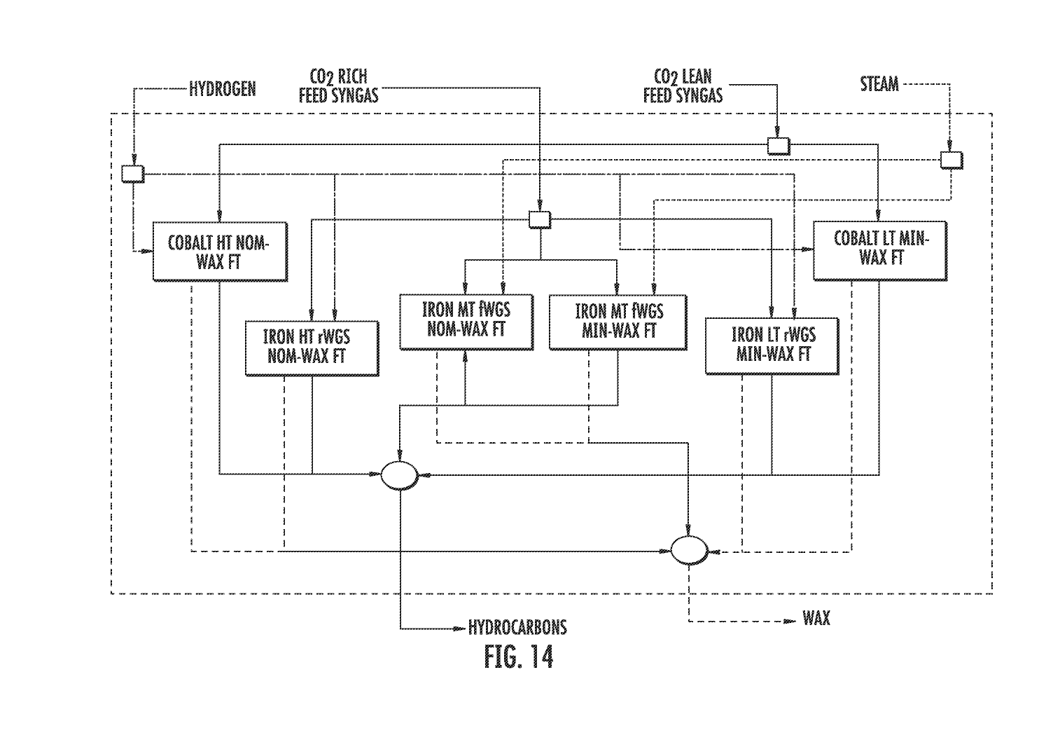

The synthesis gas is converted to a liquid stream via the Fischer-Tropsch synthesis or methanol synthesis in the liquid fuels production section. An example of this section is shown in FIG. 13 and a detailed example of a Fischer-Tropsch synthesis section is shown in FIG. 14. The product from the Fischer-Tropsch synthesis section may be directed to either a separations based upgrading or a ZSM-5 catalytic upgrading section while any methanol may either be converted to gasoline or to a distillate via conversion over a ZSM-5 catalyst or conversion to olefins followed by subsequent conversion over the ZSM-5 catalyst, respectively. Examples of typical hydrocarbons may be liquid fuels such as gasoline, diesel, or kerosene. The new processes may be an improvement on current refineries based on (I) the possibility to produce any combination of gasoline, diesel, or kerosene fuels and (II) the use of one or multiple technologies to convert the synthesis gas to the final liquid product.

Examples of technologies present in part (II) include six Fischer-Tropsch reactors operating at three different temperatures and using either cobalt or iron catalyst, the capability to upgrade the raw hydrocarbons produced in the Fischer-Tropsch reactors using a ZSM-5 catalyst or a series of treatment units including a hydrocracker, a reformer, hydrotreaters, isomerizers, and an alkylation unit, a methanol synthesis reactor to produce methanol for sale as a byproduct or use as an intermediate, a methanol to gasoline reactor to convert intermediate methanol to gasoline, and a methanol to olefins and diesel/kerosene reactor to convert intermediate methanol to diesel and kerosene.

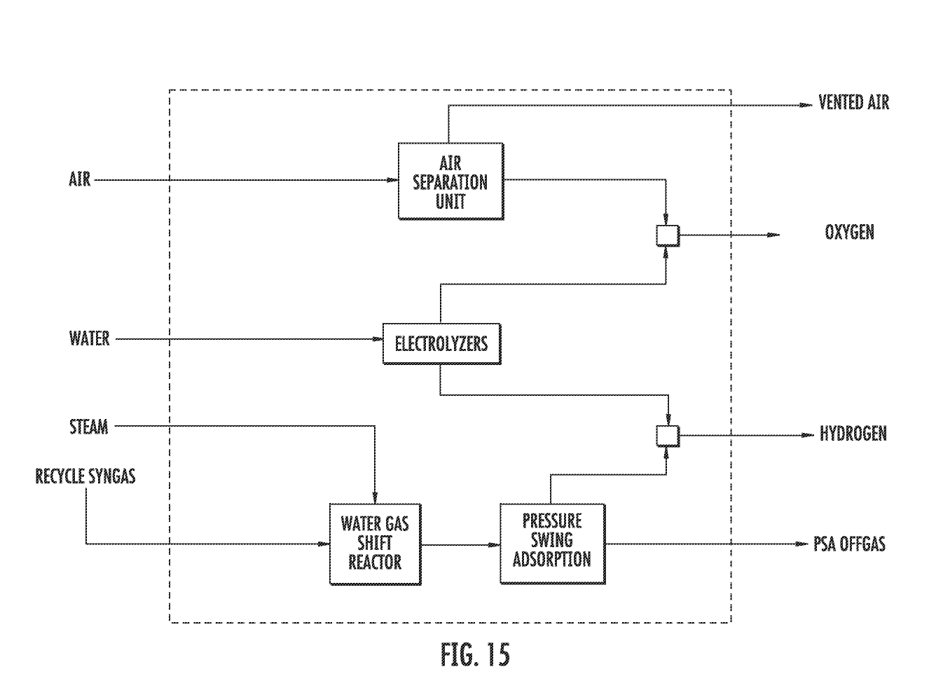

Hydrogen and oxygen production for the refinery is shown in FIG. 15. The hydrogen in the refinery can be produced through pressure-swing adsorption or via electrolysis of water. Hydrocarbon-rich light gases may be fed to the pressure-swing adsorption unit to produce a near-100% hydrocarbon stream while the electrolyzer may input freshwater or recycle process water. The oxygen for the system can be provided by the electrolyzer unit or a separate air separation unit which may be utilized to produce a high-purity oxygen stream.

The new processes may be used to help increase the marketability of natural gas resources by converting the gas into liquid products that are more readily transportable to locations that are distant from the natural gas source location (e.g., stranded natural gas, associated natural gas). The new processes have immediate application in key areas worldwide where natural gas feedstocks are abundant, have a low purchase cost, or have minimal marketable value. However, it can be used at any location to produce a desired quantity of liquid fuels. The applicability of the new processes may increase in the future with (i) increasing cost of crude oil, (ii) enhanced government initiatives to produce liquid fuels from alternative sources, (iii) increasing natural gas availability, (iv) decreasing natural gas cost, and (v) decreasing investment cost of unit operations.

The process synthesis model represents a efficient and robust methodology for directly comparing the technoeconomic and environmental tradeoffs between natural gas conversion technologies. The model therefore offers several advantages over standard natural gas to liquids refinery designs. The process synthesis model is capable of analyzing thousands of distinct process designs simultaneously to identify a singular process topology that may be mathematically guaranteed to be superior to all other considered designs. This capability offers a substantial reduction in manpower and computational effort that is required when different process designs must be investigated to minimize the capital and operating cost or maximize the annual profit. Additionally, the process topologies that are selected by the model represent novel designs that may not be considered during a typical design-stage analysis.

Novel features within the GTL refineries that are selected by the process synthesis model may include (i) the ability to use one or a combination of natural gas conversion technologies to directly or indirectly produce liquid hydrocarbons, (ii) a reverse water-gas-shift reactor to consume CO₂ using produced hydrogen, (iii) recycle of CO₂ throughout the process to consume additional CO₂ within various process units, (iv) a combination of Fischer-Tropsch units using multiple temperature levels and either iron or cobalt catalysts to produce different hydrocarbon effluent compositions, (v) a combination of a ZSM-5 catalyst or a series of hydrocracker, hydrotreater, isomerizer, and alkylation units to produce gasoline, diesel, and kerosene, (vi) a methanol synthesis reactor to produce byproduct or intermediate methanol, (vii) a combination of methanol to gasoline or methanol to diesel and kerosene units to produce the liquid fuels, (viii) a hydrogen/oxygen production system including an air separation unit, a pressure-swing adsorption unit, and electrolyzer units that is capable of producing hydrogen and oxygen from both carbon and non-carbon based sources, and (ix) a utility plant that will produce electricity and process heat using a gas turbine, a steam turbine, and a series of heat exchangers.

The new processes may provide a method for economically utilizing small quantities of natural gas that have minimal marketable value or large quantities of natural gas in remote areas that must be processed to generated liquefied natural gas. Utilization of low cost natural gas provides a means for generating high profit margins and a substantial return on the capital investment. The GTL refineries may have at most an equivalent level of life-cycle greenhouse gas emissions when compared to petroleum refineries or natural gas-based electricity. The GTL refineries may offer both an environmental and economic advantage to some alternative sources of crude that require additional costs and emissions to produce.

The processes may offer the capability to convert any fraction of the input carbon in the natural gas to liquid fuels. The new processes are capable of directly analyzing economic tradeoffs between using feedstock to produce either liquid fuels or byproduct electricity when given a minimum threshold of carbon conversion. Another advantage is the capability of producing liquid fuels using a variety of process technologies. Current processes utilize only a small number of these technologies within the plant design and may ultimately lead to inefficient process designs. The new processes may produce a more efficient design based on the inclusion of additional process considerations.

The new processes may include a (1) process synthesis model that can simultaneously analyze several process designs to determine the refinery that can produce liquid fuels at the lowest cost and (2) all novel process topologies that result from the use of the model in (1). The new processes are capable of determining the optimal composition of unit operations designed natural gas to liquid products (e.g., gasoline, diesel, kerosene, LPG). The process topologies involve six distinct stages including (i) natural gas cleanup, (ii) natural gas conversion to hydrocarbons or intermediate species, (iii) intermediate product conversion to hydrocarbons, (iv) hydrocarbon upgrading for liquid fuels production, (v) recycle gas handling, and (vi) hydrogen/oxygen production. This is shown as a topological superstructure in FIG. 10.

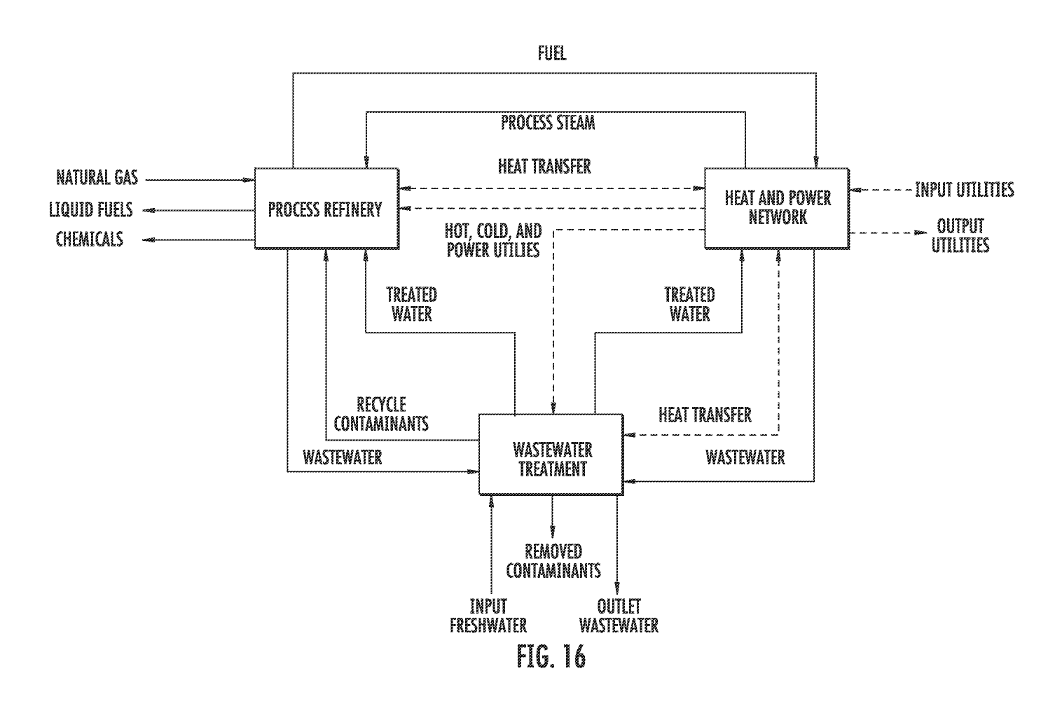

In addition to the set of unit operations detailed above for the process refinery, a combined heat, power, and water integration may also be included, as shown in FIG. 16. Heat may be transferred from the process refinery and a wastewater treatment section via a heat and power network which may be used to generate hot, cold, and power utilities needed for the process refinery and wastewater treatment. Fuel gas may also be provided from the process refinery for utility generation and may include natural gas or recycle gas from the process refinery. Excess utilities may be output from the process and sold as a byproduct and utilities may also be purchased if necessary. Wastewater produced from the process refinery and the heat and power network is directed to the wastewater treatment section where contaminants may be removed from the water and either recycled back to the refinery or removed from the system. Treated water is sent to the process refinery or to the heat and power network. Any steam needed for the process refinery may be generated from the heat and power network.

Natural gas is converted via reforming to synthesis gas (e.g., auto-thermal reforming, steam reforming, compact reforming, or CO₂ reforming), direct conversion to methanol (e.g., partial oxidation), or direct conversion to hydrocarbons (e.g., oxidative coupling to form olefins or oxychloroination to form chloronidated hydrocarbons). The synthesis gas may be passed through a forward/reverse water-gas-shift unit to alter the ratio of H₂ to CO/CO₂ in the feed. The synthesis gas may also be passed over a CO₂ removal unit (e.g., physical adsorption via methanol or amine separation) to remove a substantial portion of the CO₂ from the gas stream. CO₂ may be vented to the atmosphere, recycled to other process units in the refinery, or compressed for sequestration. The synthesis gas may be converted to (1) a methanol intermediate via a methanol synthesis or (2) hydrocarbons via Fischer-Tropsch synthesis. One or multiple Fischer-Tropsch reactor types can be utilized to produce a raw hydrocarbon composition that may be upgraded to liquid product.

The methanol produced from direct conversion of the natural gas may be combined with the methanol from the synthesis gas for conversion to liquid hydrocarbons. The methanol may be converted to gasoline-range hydrocarbons or to olefins via a ZSM-5 zeolite catalyst. The composition of hydrocarbon products from the catalytic conversion of methanol can be dependent on the operating conditions within the zeolite. Methanol may also be sold as a byproduct after separation of the entrained water.

The hydrocarbons produced from direct conversion of natural gas, Fischer-Tropsch synthesis, or methanol conversion may then be upgraded to final hydrocarbon products. The hydrocarbons may be converted to a high quality gasoline-range fraction with high yield via a ZSM-5 zeolite catalyst. Alternatively, the hydrocarbons may be fractionated using a distillation column and upgraded to gasoline, diesel, kerosene, or LPG using a combination of upgrading units including hydrocrackers, hydrotreaters, isomerizers, reformers, alkylation units, and additional distillation columns.

Recycle gases generated from various units throughout the refinery may be sent to section (ii) for additional production of hydrocarbons and intermediates, to section (iii) for conversion of intermediates to hydrocarbons, or section (vi) for hydrogen production. The hydrogen in the refinery can be produced through a pressure-swing adsorption unit or via an electrolyzer unit in section (vi). Hydrocarbon-rich light gases may be fed to the pressure-swing adsorption unit to produce a near-100% hydrocarbon stream while the electrolyzer may input freshwater or recycle process water. The oxygen for the system can be provided by the electrolyzer unit or a separate air separation unit which may be utilized to produce a high-purity oxygen stream.

Selection of the process units within the optimal refineries may be limited to the set of design alternatives considered within the process synthesis framework. That is, the process synthesis framework may only be capable of analyzing processes that have operational and cost data that are publicly known via governmental or academic studies. However, this limitation is easily overcome by extending the refinery design alternatives to include specific process units that may fulfill the desired goal.

Operational capability of the units has been taken from literature data and the results of advanced simulation methods and optimization approaches developed in house. For all units, mathematical models were developed to calculate the flow rate and composition of all streams exiting the unit given the stream inputs and operating conditions of the unit.

Embodiments include a superstructure. The superstructure may include at least one synthesis gas production unit configured to produce at least one synthesis gas selected from the group consisting of a biomass synthesis gas production unit, a coal synthesis gas production unit and a natural gas synthesis gas production unit, wherein the at least one synthesis gas is determined by a mixed-integer linear optimization model solved by a global optimization framework; a synthesis gas cleanup unit configured to remove undesired gases from the at least one synthesis gas; a liquid fuels production unit configured selected from the group including a Fischer-Tropsch unit, the Fischer-Tropsch unit being configured to produce a first output from the at least one synthesis gas, and a methanol synthesis unit, the methanol synthesis unit being configured to produce a second output from the at least one synthesis gas, wherein the selection of liquid fuels production unit is determined by the mixed-integer linear optimization model solved by the global optimization framework; a liquid fuels upgrading unit configured to upgrade the first output or the second output, wherein the liquid fuels upgrading unit is determined by the mixed-integer linear optimization model solved by the global optimization framework; a hydrogen production unit configured to produce hydrogen for the refinery; an oxygen production unit configured to produce oxygen for the refinery; a wastewater treatment network configured to process wastewater from the refinery and input freshwater into the refinery, wherein the wastewater treatment network is determined by a mixed-integer linear optimization model solved by a global optimization framework; a utility plant configured to produce electricity for the refinery and process heat from the refinery, wherein the utility plant is determined by a mixed-integer linear optimization model solved by a global optimization framework; and a CO₂ separation unit configured to recycle gases containing CO₂ in the refinery. The at least one synthesis gas production unit, the synthesis gas cleanup unit, the liquid fuels production unit, the liquid fuels upgrading unit, the hydrogen production unit, the oxygen production unit, the wastewater treatment network, the utility plant and the CO₂ separation unit may be configured to be combined to form the refinery.

An embodiment includes an optimal refinery design system. The optimal refinery design system may include a superstructure database. The superstructure database may include data associated with at least one synthesis gas production unit configured to produce at least one synthesis gas selected from the group consisting of a biomass synthesis gas, a coal synthesis and a natural gas synthesis gas. The selection of the at least one synthesis gas may be determined by a mixed-integer linear optimization model solved by a global optimization framework. A synthesis gas production unit configured to produce biomass synthesis gas may be referred to as a biomass synthesis gas production unit. A synthesis gas production unit configured to produce coal synthesis gas may be referred to as a coal synthesis gas production unit. A synthesis gas production unit configured to produce natural gas may be referred to as a natural gas synthesis production unit. The superstructure database may also include data associated with a synthesis gas cleanup unit configured to remove undesired gases from the at least one synthesis gas. The superstructure database may also include data associated with a liquid fuels production unit configured selected from the group including a Fischer-Tropsch unit and a methanol synthesis unit. The Fischer-Tropsch unit may be configured to produce a first output from the at least one synthesis gas. The methanol synthesis unit may be configured to produce a second output from the at least one synthesis gas. The selection of liquid fuels production unit is determined by the mixed-integer linear optimization model solved by the global optimization framework. The superstructure database may also include data associated with a liquid fuels upgrading unit configured to upgrade the first output or the second output. The selection of the liquid fuels upgrading unit may be determined by the mixed-integer linear optimization model solved by the global optimization framework. The superstructure database may also include data associated with a hydrogen production unit configured to produce hydrogen for the refinery; an oxygen production unit configured to produce oxygen for the refinery; and a wastewater treatment network configured to process wastewater from the refinery and input freshwater into the refinery. The wastewater treatment network is determined by the mixed-integer linear optimization model solved by the global optimization framework. The superstructure database may also include data associated with a utility plant configured to produce electricity for the refinery and process heat from the refinery. The utility plant is determined by the mixed-integer linear optimization model solved by the global optimization framework. The superstructure database may also include data associated with a CO₂ separation unit configured to recycle gases containing CO₂ in the refinery. The at least one synthesis gas production unit, the synthesis gas cleanup unit, the liquid fuels production unit, the liquid fuels upgrading unit, the hydrogen production unit, the oxygen production unit, the wastewater treatment network, the utility plant and the CO₂ separation unit may be configured to be combined to form the refinery. The optimal refinery design system may include a processor configured to solve the mixed-integer linear optimization model by the global optimization framework.

The biomass synthesis gas production unit may be a biomass gasification unit. The coal synthesis gas production unit may be a coal gasification unit. The natural gas synthesis gas production unit may be generated a natural gas auto-thermal reforming unit.

The synthesis gas cleanup unit may include one or more of a hydrolyzer, a scrubber, a rectisol unit, a strupper column, and a claus recovery system.

The liquid fuels product unit may be a Fischer-Tropsch unit. The Fischer-Tropsch unit is selected from the group consisting of a low temperature cobalt catalyst Fischer-Tropsch unit; a high temperature cobalt catalyst Fischer-Tropsch unit; a medium temperature low wax iron catalyst Fischer-Tropsch unit; a medium temperature high wax iron catalyst Fischer-Tropsch unit; a high temperature iron catalyst Fischer-Tropsch unit; and a low temperature iron catalyst Fischer-Tropsch unit.

The first output may be raw hydrocarbons. The second output may be methanol.

The liquid fuels upgrading unit may be a ZSM-5 catalytic reactor. The liquid fuels upgrading unit may be a series of hydrotreating units, a wax hydrocracker, two isomerization units, a naphtha reformer, an alkylation unit and a gas separation plant.

The liquid fuels production unit may be a methanol synthesis unit. The liquid fuels upgrading unit may be a methanol-to-gasoline reactor. The liquid fuels upgrading unit may be a methanol-to-olefins reactor and a mobil olefins-to-gasoline/distillate reactor.

The hydrogen production unit may be a pressure swing adsorption unit. The hydrogen production unit may be an electrolyzer unit.

The oxygen production unit may be an electrolyzer unit. The oxygen production unit may be a distinct air separation unit.

The utility plant may include a gas turbine, a steam turbine, and a series of heat exchangers.

An embodiment includes a method of designing an optimal refinery. The method may include providing any superstructure contained herein; inserting a data set on each of the at least one synthesis gas production unit, the liquid fuels production unit, the liquid fuels upgrading unit, the wastewater treatment network and the utility plant into the mixed-integer linear optimization model and solving the mixed-integer linear optimization model by the global optimization framework. The method thereby determining each of the at least one synthesis gas production unit, the liquid fuels production unit, the liquid fuels upgrading unit, the wastewater treatment network and the utility plant to produce an optimal refinery design.

An embodiment includes a method of designing an optimal refinery. The method may include providing any superstructure database contained herein; solving the mixed-integer linear optimization model by the global optimization framework; and determining each of the at least one synthesis gas production unit, the liquid fuels production unit, the liquid fuels upgrading unit, the wastewater treatment network and the utility plant to include in the optimal refinery design.

An embodiment includes a method of producing liquid fuels. The method may include producing liquid fuels by an optimal refinery design. The optimal refinery design may be arrived at by providing any superstructure herein; inserting a data set on each of the each of the at least one synthesis gas production unit, the liquid fuels production unit, the liquid fuels upgrading unit, the wastewater treatment network and the utility plant into the mixed-integer linear optimization model; solving the mixed-integer linear optimization model by the global optimization framework; and determining each of the at least one synthesis gas production unit, the liquid fuels production unit, the liquid fuels upgrading unit, the wastewater treatment network and the utility plant to include in the optimal refinery design.

The method may include providing a superstructure database; solving the mixed-integer linear optimization model by the global optimization framework; determining each of the at least one synthesis gas production unit, the liquid fuels production unit, the liquid fuels upgrading unit, the wastewater treatment network and the utility plant to produce an optimal refinery design; and producing liquid fuels by the optimal refinery design.

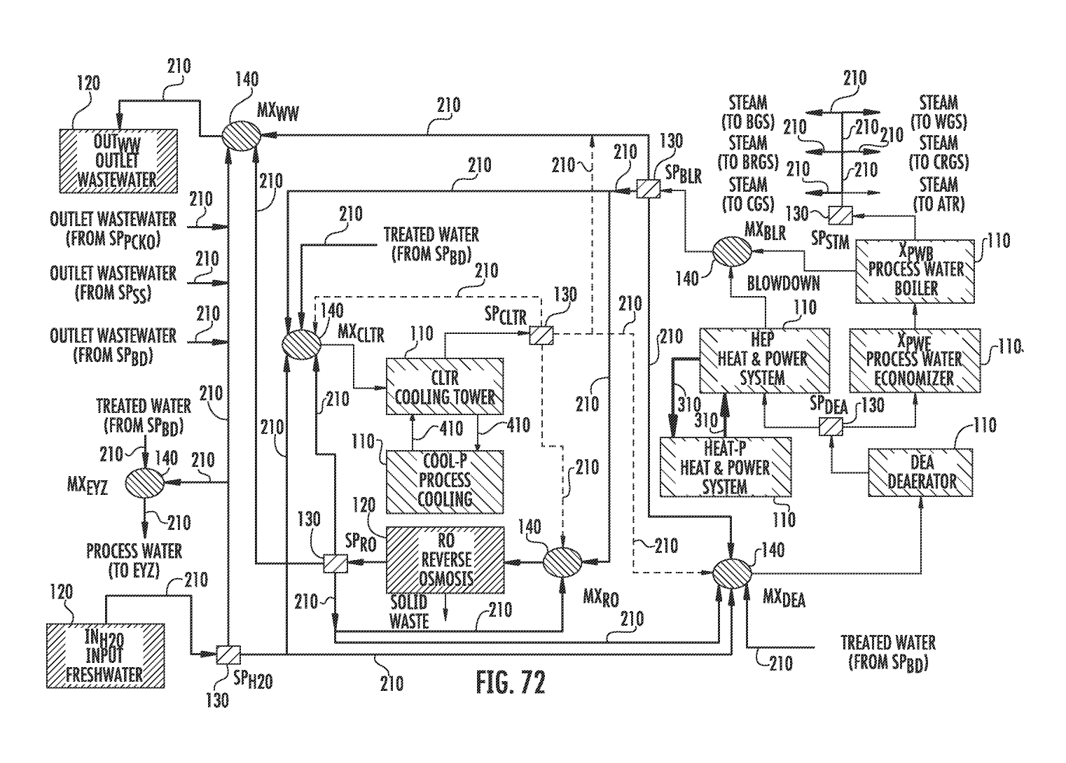

A computing device may be used to implement features described herein with reference to FIGS. 1-72. An example computing device includes a processor, memory device, communication interface, peripheral device interface, display device interface, and data storage device. A display device may be coupled to or included within the computing device. Embodiments include a computing device configured to implement methods herein, a computer-readable medium including processor-executable instructions to conduct a method herein, and computer implemented methods.

The memory device may be or include a device such as a Dynamic Random Access Memory (D-RAM), Static RAM (S-RAM), or other RAM or a flash memory. The data storage device may be or include a hard disk, a magneto-optical medium, an optical medium such as a CD-ROM, a digital versatile disk (DVDs), or Blu-Ray disc (BD), or other type of device for electronic data storage.

The communication interface may be, for example, a communications port, a wired transceiver, a wireless transceiver, and/or a network card. The communication interface may be capable of communicating using technologies such as Ethernet, fiber optics, microwave, xDSL (Digital Subscriber Line), Wireless Local Area Network (WLAN) technology, wireless cellular technology, and/or any other appropriate technology.

The peripheral device interface may be configured to communicate with one or more peripheral devices. The peripheral device interface operates using a technology such as Universal Serial Bus (USB), PS/2, Bluetooth, infrared, serial port, parallel port, and/or other appropriate technology. The peripheral device interface may, for example, receive input data from an input device such as a keyboard, a mouse, a trackball, a touch screen, a touch pad, a stylus pad, and/or other device. Alternatively or additionally, the peripheral device interface may communicate output data to a printer that is attached to the computing device via the peripheral device interface.

The display device interface may be an interface configured to communicate data to display device. The display device may be, for example, a monitor or television display, a plasma display, a liquid crystal display (LCD), and/or a display based on a technology such as front or rear projection, light emitting diodes (LEDs), organic light-emitting diodes (OLEDs), or Digital Light Processing (DLP). The display device interface may operate using technology such as Video Graphics Array (VGA), Super VGA (S-VGA), Digital Visual Interface (DVI), High-Definition Multimedia Interface (HDMI), or other appropriate technology. The display device interface may communicate display data from the processor to the display device for display by the display device. The display device may be external to the computing device, and coupled to the computing device via the display device interface. Alternatively, the display device may be included in the computing device.

An instance of the computing device may be configured to perform any feature or any combination of features described herein. Alternatively or additionally, the memory device anchor the data storage device may store instructions which, when executed by the processor, cause the processor to perform any feature or any combination of features described herein. Alternatively or additionally, each or any of the features described herein may be performed by the processor in conjunction with the memory device, communication interface, peripheral device interface, display device interface, and/or storage device.

A tablet computer is a more specific example of the computing device. The tablet computer may include a processor (not depicted), memory device (not depicted), communication interface (not depicted), peripheral device interface (not depicted), display device interface (not depicted), storage device (not depicted), and touch screen display, which may possess characteristics of the processor, memory device, communication interface, peripheral device interface, display device interface, storage device, and display device, respectively, as described above. The touch screen display may receive user input using technology such as, for example, resistive sensing technology, capacitive sensing technology, optical sensing technology, or any other appropriate touch-sensing technology.

As used herein, the term “processor” broadly refers to and is not limited to a single- or multi-core processor, a special purpose processor, a conventional processor, a Graphics Processing Unit (GPU), a digital signal processor (DSP), a plurality of microprocessors, one or more microprocessors in association with a DSP core, a controller, a microcontroller, one or more Application Specific Integrated Circuits (ASICs), one or more Field Programmable Gate Array (FPGA) circuits, any other type of integrated circuit (IC), a system-on-a-chip (SOC), and/or a state machine.

As used to herein, the term “computer-readable medium” broadly refers to and is not limited to a register, a cache memory, a ROM, a semiconductor memory device (such as a D-RAM, S-RAM, or other RAM), a magnetic medium such as a flash memory, a hard disk, a magneto-optical medium, an optical medium such as a CD-ROM, a DVDs, or BD, or other type of device for electronic data storage.

Although features are described herein as being performed in a computing device, the features described herein may also be implemented, mutatis mutandis, on a desktop computer, a laptop computer, a netbook, a cellular phone, a personal digital assistant (PDA), or any other appropriate type of tablet computing device or data processing device. The systems and methods described herein may be performed on a single computing device or a plurality of computing devices.

Although features and elements are described above in particular combinations, each feature or element can be used alone or in any combination with or without the other features and elements. For example, each feature or element as described above may be used alone without the other features and elements or in various combinations with or without other features and elements. Sub-elements of the methods and features described above may be performed in any arbitrary order (including concurrently), in any combination or sub-combination.

An embodiment includes any superstructure as shown and/or described herein and in the accompanying drawings.

An embodiment includes any refinery design as shown and/or described herein and in the accompanying drawings.

An embodiment includes any method of designing a refinery as shown herein and in the accompanying drawings.

An embodiment includes a refinery having any refinery design as shown and/or described herein and in the accompanying drawings.

An embodiment includes any method of producing liquid fuels as shown herein and in the accompanying drawings.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

### Example 1

### Toward Novel Hybrid Biomass, Coal, and Natural Gas Processes for Satisfying Current Transportation Fuel Demands: Process Alternatives, Gasification Modeling, Process Simulation, and Economic Analysis

This example discloses a hybrid coal, biomass, and natural gas to liquids (CBGTL) process that can produce transportation fuels in ratios consistent with current U.S. transportation fuel demands. Using the principles of the H₂Car process, an almost-100% feedstock carbon conversion was attained using hydrogen produced from a carbon or noncarbon source and the reverse water-gas-shift reaction. Seven novel process alternatives that illustrate the effect of feedstock, hydrogen source, and light gas treatment on the process are considered. A complete process description is presented for each section of the CBGTL process including syngas generation, syngas treatment, hydrocarbon generation, hydrocarbon upgrading, and hydrogen generation. Novel mathematical models for biomass and coal gasification are developed to model the nonequilibrium effluent conditions using a stoichiometry-based method. Input-output relationships are derived for all vapor-phase components, char, and tar through a nonlinear parameter estimation optimization model based on the experimental results of multiple case studies. Two distinct Fischer-Tropsch temperatures and a detailed upgrading section based on a Bechtel design are used to produce the proper effluent composition to correctly match the desired ratio of gasoline, diesel, and kerosene.

Steady-state process simulation results based on Aspen Plus are presented for the seven process alternatives with a detailed economic analysis performed using the Aspen Process Economic Analyzer and unit cost functions obtained from literature. Based on the appropriate refinery margins for gasoline, diesel, and kerosene, the price at which the CBGTL process becomes competitive with current petroleum-based processes is calculated. This break-even oil price is derived for all seven process flowsheets, and the sensitivity analysis with respect to hydrogen price, electricity price, and electrolyzer capital cost, is presented.

One of the main concerns regarding bio-based feedstocks is the amount of land required to produce an adequate fraction of the transportation fuel demand. The U.S. Department of Energy (DOE) has recently addressed the feasibility of an annual supply of one billion dry tons of biomass, but it is essential to quantify the impact that this figure can have on the current demand. A lower bound on the total biomass required to satisfy all transportation fuel demand can be found through a simple carbon mass balance. The 2008 demand for gasoline, diesel, and kerosene was 8803 TBD, 2858 TBD, and 1539 TBD, respectively. Assuming that each fuel can be assigned an average density and molecular formula (see Table 1), the total carbon needed to produce the entire U.S. demand is 5.008×1011 kg/yr. If switchgrass is taken as a representative biomass compound (it has an average carbon dry wt % of 46.96), the total amount required is 1.176×1012 dry tons annually. It is evident that biomass has the capability of producing a significant fraction, if not all, of the transportation fuel requirement. However, a critical assumption here is that all of the carbon present in the biomass is converted directly into liquid fuels. This is typically not the case for current FT designs using either biomass or hybrid biomass/coal feedstocks, which only convert 33% of the total feedstock carbon to liquid fuels. The key reason for the lack of carbon conversion lies in the formation of CO₂, which must either be sequestered or vented.

TABLE 1
Estimated Carbon Flow for the 2008 Transportation Sector Demand

| | demand | density | molecular | carbon flow |
|---|---|---|---|---|
| fuel | (TBD^{a}) | (g/cm³) | formula | (kg/yr) |
| gasoline | 8803 | 0.747 | C₉H₂₀ | 3.215 × 10¹¹ |
| diesel | 7858 | 0.847 | C₁₅H₃₂ | 1.191 × 10¹¹ |
| kerosene | 1539 | 0.797 | C₁₂H₂₆ | 6.021 × 10¹⁰ |
| total | | | | 5.008 × 10¹¹ |

^{a}TBD = thousand barrels per day.

In light of the aforementioned issues, studies have been conducted to explore alternative, non-petroleum-based processes to produce liquid fuels that include the production of FT liquids from biomass (BTL), coal (CTL), and natural gas (GTL) (Kreutz et al, 2008; Larson and Jin, 1999; Vliet et al., 2009; USDOE contract No. DE-AC26-99FT40342, 2003, which are incorporated herein by reference as if fully set forth). Synthetic gas (syngas) is produced via natural gas reforming, which is a well-known and industrially applied technology, or via coal and biomass gasification (Vliet et al., 2009; Sudiro and Bertucco, 2009, which are incorporated herein by reference as if fully set forth). Furthermore, hybrid processes that combine features of these processes have also been investigated. Kreutz et al., 2008, which is incorporated herein by reference as if fully set forth, studied 16 configurations of CTL, BTL, and a combined coal and biomass process (CBTL). Particular attention was given to the CBTL process, because of its potential net-zero GHG emission to the atmosphere (i.e., when the release of CO₂ to the atmosphere is equal to CO₂ in-take during photosynthesis). Cao et al., 2008, which is incorporated herein by reference as if fully set forth, combined CTL and GTL by injecting methane to the gasification reactor and reported a synergistic effect in producing syngas with a H₂:CO ratio of ˜2, which is the stoichiometric requirement of the FT process. Sudiro and Bertucco, 2007, which is incorporated herein by reference as if fully set forth, coupled the steam reforming of natural gas and the steam gasification of coal in a reactor that uses solar energy as a heat source. In another process, Sudiro and Bertucco, 2009, which is incorporated herein by reference as if fully set forth, used separate gasification and reforming processes with CO₂ recycle to the gas reforming block and observed a reduction in CO₂ emissions from the CTL case. Note that these BTL, CTL, and GTL technologies can also co-produce hydrogen and electricity (Yamashita and Barreto, 2005; Chiesa et al., 2005; Kreutz et al., 2005; Sudiro et al., 2008; Larson et al., 2009; Cormos, 2009; Jimenez et al., 2009, which are incorporated herein by reference as if fully set forth).

The common feature of many FT-based processes, however, is the large CO₂ emissions from the system. Although these studies achieved a reduction in GHG emissions, the processes either vent the produced CO₂ or reduce emissions using carbon capture and storage (CCS) technology. Recently, a novel process was proposed, denoted as the H₂Car process (Agrawal et al., 2007, which is incorporated herein by reference as if fully set forth), and its capabilities of obtaining an almost-100% conversion of the feedstock carbon using hydrogen that has been derived from a noncarbon source were shown. Using either wind, solar, or nuclear energy, hydrogen can be generated from water and reacted with CO₂, utilizing the reverse water-gas-shift (RGS) reaction. The CO generated from the reaction can then be sent to the FT unit to recover additional liquid fuels. It is important to note that if the hydrogen does not come from a carbon-free source, then it is not possible to claim an almost-100% carbon conversion due to the sequestration required from the production of hydrogen. However, hydrogen production from a carbon source (i.e., steam reforming of methane (SRM)) is still a viable option, because current large-scale production of hydrogen from noncarbon sources is hindered by the large capital costs associated with wind turbines, solar panels, nuclear plants, and electrolyzers. These alternatives may be economical in the future and should still be considered as technology alternatives. Using hydrogen from SRM still achieves an almost-100% conversion of the biomass feedstock, significantly reducing the land area requirement for feedstock production.

The production of gasoline, diesel, and kerosene in mass ratios consistent with the U.S. transportation demand, was investigated, based on the principles of the H₂Car process. The process will use a carbon-based feedstock consisting of Illinois No. 6 coal, herbaceous biomass, and natural gas to produce the liquid fuels (coal, biomass, and natural gas to liquids (CBGTL)). Hydrogen will be produced off-site from a carbon-based source or on-site using electrolyzers. The conceptual design of the CBGTL process, is described herein. Seven process design alternatives are described in full detail and simulated with the Aspen Plus v7.1 package. Detailed mathematical modeling of several key process units is described, namely, the novel biomass and coal nonequilibrium, stoichiometry-based gasifier models. A nonlinear parameter estimation is performed to match the theoretical output of the gasifiers with several reported experimental case studies. Results on the simulations of the seven process alternatives are presented, and a simultaneous heat and power integration is performed as detailed in Example 2. Finally, a detailed economic analysis is conducted to determine the price of crude oil at which the CBGTL process is competitive with current petroleum-based processes. In Example 2, the steps to fully heat and power integrate each of the seven process alternatives are outlined. The steps include the minimization of the utility/power cost, followed by minimization of the annualized cost of heat exchange. A novel heat and power integration model is developed using heat engines to ensure optimal recovery of the electricity and cooling water utilities.

### Example 1.1

### Conceptual Design of the CBGTL Process

The CBGTL process is designed to co-feed a carbon source such as biomass, coal, or natural gas, as well as H₂ to produce transportation fuel with 100% carbon conversion. Gasification technology is utilized to produce syngas from biomass and coal, which is then converted to hydrocarbon products in the FT reactors. Co-feeding of biomass and coal to the process is done through distinct, parallel biomass and coal gasification trains, followed by subsequent mixing of the individual syngas effluent streams. The natural gas feedstock enters downstream of the FT units in an autothermal reactor (ATR), where it is combined with the residual light hydrocarbons from the FT reaction.

To provide the 2:1 H₂:CO molar ratio for optimal carbon conversion in the FT unit, the syngas composition from the gasification section may be shifted. A reverse water-gasshift (RGS) reactor is introduced to obtain the desired ratio via the RGS reaction and the addition of H₂ while simultaneously reducing the CO₂ concentration. This enables a closed-loop system where all CO₂ streams from various sections of the process are recycled into the RGS unit, shifted to CO, and subsequently converted to hydrocarbon products in the FT reactors. The resulting effect is a very high carbon conversion from feedstock to product and a very low CO₂ emission from the process, eliminating the need for CO₂ sequestration. The H₂ required for the RGS reaction can be produced by steam reforming of methane or on-site electrolysis, which affects the overall capital cost, as well as the production of O₂. While electrolysis will provide pure O₂ along with H₂, processes producing H₂ from a carbon source may require the addition of an air separation unit (ASU) to produce pure O₂. The O₂ produced in the former case can be sold for a profit, but market saturation will rapidly occur when the process is scaled up.

It is also desirable to strip CO₂ and sulfur components from the syngas to increase the partial pressure of the reactants before sending them into the FT reactor. This cleaning process is facilitated by a series of syngas treatment units, including (i) a hydrolyzer to shift COS and HCN to H₂S and NH₃, respectively, 27 (ii) a scrubber to remove HCl and NH₃, (iii) a two stage Rectisol unit to separate CO₂ and H₂S from the stream, (iv) a stripper column to remove sour gas from the plant's disposed water, and (v) a Claus recovery system to extract elemental sulfur from the syngas. The CO₂ stream is then compressed and sent back to the RGS unit while the clean, CO₂-free and sulfur-free syngas is sent to the FT section.

To produce gasoline, diesel, and kerosene products according to the U.S. mass demand ratio, we employ FT reactors operating at two different conditions: FT reactors at high temperature (320° C.) and low temperature (240° C.), each associated with distinct R (chain growth probability measure) values. This R value is the single parameter used to predict the entire range of hydrocarbon products in the modeling of a FT reactor. The syngas is split such that the varied hydrocarbon product distributions given from the two R values result in the correct product ratio. Fuel quality products are obtained by treating the FT effluents in a detailed upgrading section. A hydrocracker unit is present to convert waxes to additional fuels, and hydrotreater units are employed to upgrade the naphtha and distillate fractions. The naphtha cut is further reformed and isomerized to improve the octane number. Lighter forms of hydrocarbons are passed through a series of alkylation and isomerization processes to form high-octane gasoline blending stock. The off-gases from various upgrading units are combined in a saturated gas plant and reformed in the following three alternatives: (i) an ATR unit, (ii) a combustion unit, and (iii) a gas turbine engine. The fraction to the combustion unit is determined to satisfy the fuel requirement of the plant. The remaining gases are either sent to a gas turbine engine, where they are combusted and expanded to produce electricity, or to the ATR for steam reforming. The ATR unit is where the natural gas feedstock is introduced into the process. Effluents of the combustion unit and the gas turbine engine are passed through a one-stage Rectisol unit to separate out CO2 from the build-up nitrogen. The CO₂ stream, along with effluent of the ATR, are recycled back to the RGS unit, minimizing CO₂ emission from the process.

### Example 1.2

### CBGTL Process Description

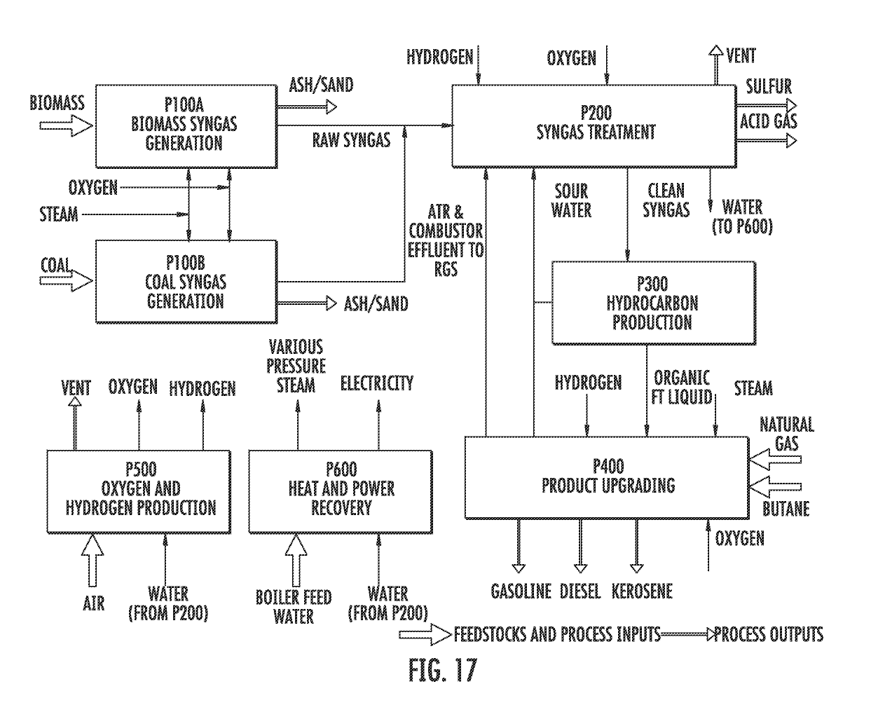

Using several key unit operations that have been reported in the literature (National Research Council and National Academy of Engineering, 2004; Kreutz et al., 2008; Vliet et al., 2009; Agrawal et al., 2007; National Energy Technology Laboratory, 2007; Bechtel, 1998; Bechtel, 1992; Hamelunck, 2004, which are incorporated herein by reference as if fully set forth), a process flowsheet is generated and developed in Aspen Plus. The CBGTL process is designed to fulfill the mass ratio of U.S. transportation fuel needs for gasoline, diesel, and kerosene, by taking combinations of biomass, coal, and natural gas as feedstock. Referring to FIG. 17, the developed process flowsheet consists of the following main sections: (i) syngas generation (P100), (ii) syngas treatment (P200), (iii) hydrocarbon production (P300), (iv) hydrocarbon upgrading (P400), (v) oxygen and hydrogen production (P500), and (vi) heat and power recovery (P600). The thermodynamics package for the Peng-Robinson equation of state with the Boston-Mathias alpha function is used in the simulation. The enthalpy model used for nonconventional components in the flowsheet (i.e., biomass, coal, ash, and char) is HCOALGEN, and the density model DCOALIGT is used for biomass and coal and DCHARIGT is used for ash and char.

Details on the list of units, Aspen Plus modules used, and their operating conditions are available in Tables 70-73.

### Example 1.3

### Syngas Generation (Area P100)

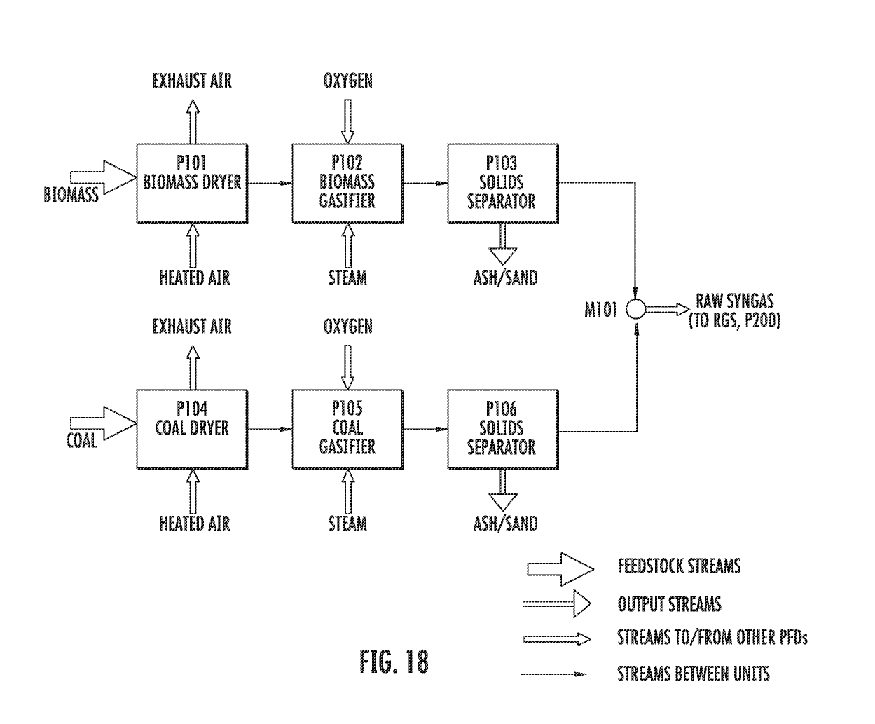

Biomass and coal are converted to syngas using distinct, parallel gasification trains (see FIG. 18). It has been estimated that 416 million dry tons of biomass are available annually, which would supply 35% of the transportation demand on a carbon basis. Therefore, a hybrid feedstock is developed from biomass, coal, and natural gas, so that 35% of the transportation demand is satisfied by biomass, 40% is supplied by coal, and 25% is supplied by natural gas. Assuming a total carbon feedstock input of 2000 tonnes per day (TPD), a total of 948.62 TPD of biomass and 678.87 TPD of coal are fed to the gasifiers. The 372.51 TPD of natural gas is input to an ATR unit in the hydrocarbon upgrading section. The feedstock properties can be found in Tables 2 and 3.

TABLE 2
Feedstock Properties

| parameter | coal | biomass |
|---|---|---|
| proximate analysis, wt % | | |
| moisture (ar^{a}) | 8.60 | 15.00 |
| ash (db^{b}) | 11.49 | 6.19 |
| volatile matter (db) | 42.23 | 42.5 |
| fixed carbon (db) | 46.28 | 21.31 |
| ultimate analysis (daf^{c}), wt % | | |
| C | 80.23 | 50.06 |
| H | 5.42 | 6.10 |
| N | 1.58 | 0.92 |
| S | 3.60 | 0.10 |
| Cl | 0.11 | 0.00 |
| O (by difference) | 9.06 | 42.82 |
| higher heating value, HHV (MJ/kg) | 27.114 | 15.935 |

^{a}ar = as received,
^{b}db = dry basis.
^{c}daf = dry, ash free.

TABLE 3
Natural Gas Composition

| component | amount (mol %) |
|---|---|
| methane | 95.2 |
| ethane | 2.5 |
| propane | 0.2 |
| isobutane | 0.03 |
| n-butane | 0.03 |
| isopentane | 0.01 |
| n-pentane | 0.01 |
| nitrogen | 1.3 |
| CO₂ | 0.7 |
| O₂ | 0.02 |

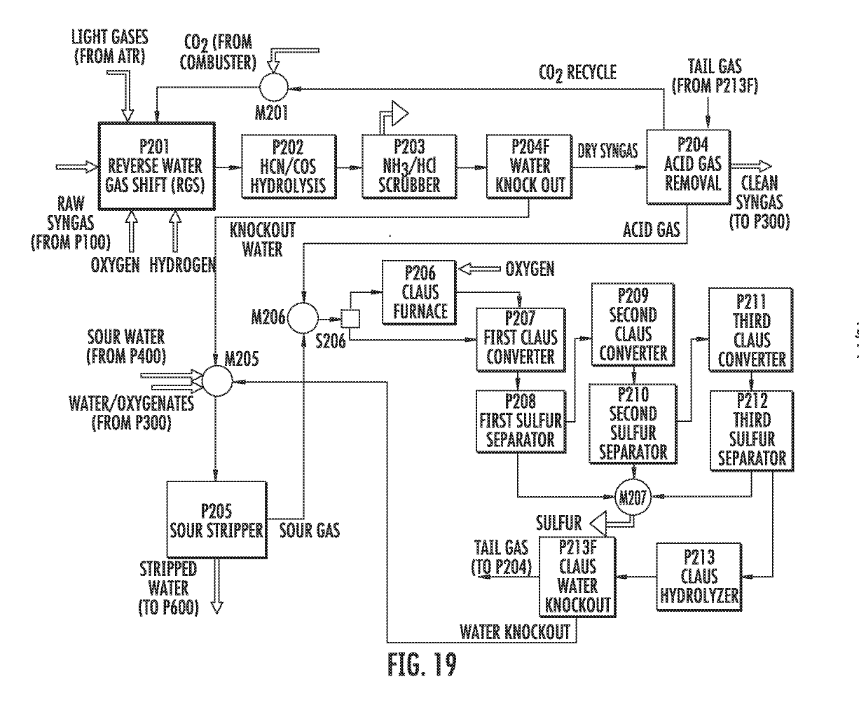

Herbaceous biomass feedstock is sent to a biomass dryer (P101), where heated air reduces the biomass moisture content to 15 wt %. The inlet air is preheated to 450° F., and its flow rate is adjusted to ensure a zero-net heat duty within the dryer unit. The moist air at T) 102° C. is vented, and the dried biomass at T) 98° C. is sent to a lockhopper where CO₂ at 31 bar is used to feed the biomass to the circulating gasifier (P102) operating at 900° C. and 30 bar. This CO₂ stream is taken from the recycle stream to the RGS unit (see FIG. 19) and its flow rate is adjusted to be equal to 10 wt % of the bone-dry biomass flow rate.

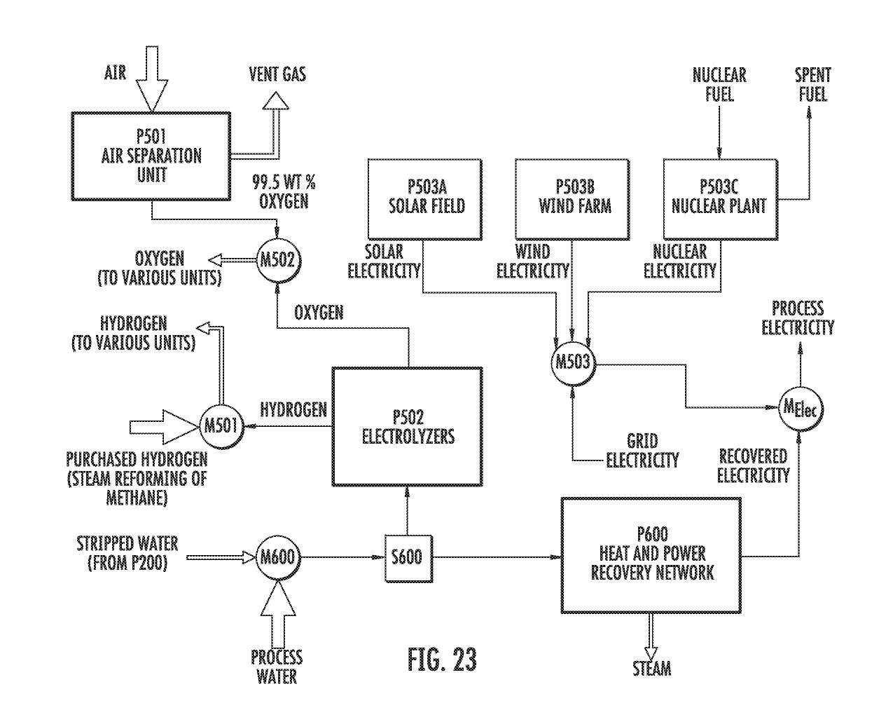

Oxygen and steam facilitate char gasification in P102, and their inlet flow rates are adjusted to maintain a mass ratio of 0.3 and 0.25, respectively, to the bone-dry biomass input. Oxygen is provided either via an ASU (P501, see FIG. 23) or the electrolyzer unit (P502), and steam is saturated at 35 bar. The gasifier unit is modeled stoichiometrically, where the syngas effluent composition is calculated based on (i) feedstock composition, (ii) input steam amount, and (iii) gasifier operating temperature, using a nonlinear optimization (NLP) model described in Example 1.10. The biomass gasifier effluent is passed through a primary and secondary cyclone, where 99% and 100% of the solid material is separated, respectively. The char is recycled back to the biomass gasifier, while the ash is purged from the system. The vapor products are sent to a tar cracker to decompose some of the residual hydrocarbons and ammonia, using the reactions listed in Table 4. The tar cracker effluent is sent to the syngas mixer (M101) before being directed to the RGS unit in the next section of the flowsheet.

TABLE 4
Reactions and Fractional Conversions for the Tar Cracker

| | fractional conversion |
|---|---|
| reaction | of main compound |
| O₂ + 2H₂ → 2H₂O. | 1 O₂ |
| CH₄ + H₂O → CO + 3H₂ | 0.5 CH₄ |
| C₂H₂ + 2H₂O → 2CO + 3H₂ | 0.5 C₂H₂ |
| C₂H₄ + 2H₂O → 2CO + 4H₂ | 0.5 C₂H₄ |
| C₂H₆ + 2H₂O → 2CO + 5H₂ | 0.9 C₂H₆ |
| 2NH₃ → N₂ + 3H₂ | 0.7 NH₃ |

The coal gasification train operates similarly to the biomass train (FIG. 18). Inlet air is preheated to dry Illinois No. 6 coal (Table 2) to 2 wt % moisture in the coal dryer (P104). The air flow rate is preheated to 450° F. and is adjusted to maintain a zero-net heat duty across the dryer. The moist air (7) 102° C.) is vented and the dried coal (T) 98° C.) is fed with pressurized CO₂ carrier gas (10 wt % of dry coal flow rate) via a lockhopper into an entrained flow gasifier (P105) operating at 1437° C. and 31 bar.27 The P105 inlet flow rates of oxygen and 35 bar of saturated steam are adjusted to maintain a mass ratio of 0.7 and 0.3, respectively, to the bone-dry coal input. The syngas exits the gasifier below the ash melting point at 891° C., after which 99% of the ash is removed as liquid slag. The syngas then enters an ash separator and a fly ash separator (P106), where 99% and 100% of solid materials are separated, respectively. The solid char is recycled back to the coal gasifier and the syngas is sent to M101.

### Example 1.4

### Syngas Treatment (Area P200)

The syngas from M101 is fed to the reverse water-gas-shift (RGS) reactor (P201) to shift the H₂:CO ratio to 2:1 in the effluent stream by H₂ addition (FIG. 19). The effluent is assumed to be in equilibrium, with respect to the RGS reaction:

CO+H₂O [symbol] CO₂+H₂ (1)

The existence of this RGS unit allows a closed-loop, CO₂ recycle system that yields almost 100% carbon conversion. The CO₂ recycle stream from the acid gas removal unit (P204), combuster (P413) and gas turbine engine (P415) along with the reformed gases from the ATR (P412) are fed to the RGS unit (FIG. 19). The unit operates at 700° C., and the only components considered in the equilibrium calculations are CO, CO₂, H₂, H₂O, and O₂. The inlet streams are preheated to a constant temperature to ensure a net-zero heat duty for the RGS reactor.

The RGS effluent is cooled to 185° C. and fed to a hydrolyzer unit (P202) to undergo the following reactions:

COS+H₂O [symbol] CO₂+H₂S (2)

HCN+H₂O [symbol] CO+NH₃ (3)

Only the components present in the above two equations will be considered in the reaction-constrained equilibrium calculations. The gas is further cooled to 35° C. and sent to a NH₃/HCl scrubber (P203), a flash unit (P204F), and a two-stage Rectisol unit (P204) combined with the tail gas from the Claus process. The Rectisol unit recovers a pure CO₂ and an acid gas stream, based on the split fractions in Table 5. The CO₂ split fraction for the clean syngas stream is adjusted to obtain a concentration of 3 mol % CO₂ in the clean syngas stream. A thermal analyzer records the thermal heat removal required to cool the inlet syngas to 12° C. This heat quantity is used to calculate the electricity requirement for refrigeration. One-third of the pure CO₂ stream is output at 1.2 bar and two-thirds is output at 3 bar. The 1.2 bar of CO₂ is compressed to 3 bar and mixed with the balance of the outlet CO₂ before being compressed to 32 bar. A fraction of the recycle CO₂ is separated for use in the gasification lockhoppers. The remaining CO₂ is preheated before being recycled back to the RGS reactor (FIG. 19).

TABLE 5
Split Fractions for the Acid Gas Unit

| outlet stream | split fraction | outlet conditions |
|---|---|---|
| clean syngas | CO₂ (3 mol %), | T = 27.2° C., P = 20.1 bar |
| | 100% of other gases | |
| pure CO₂ | balance of CO₂ | T = 25° C., P = 1.2 bar (1/3), |
| | | P = 3 bar (2/3) |
| other acid gases | 100% of: H₂S, SO₂, | T = 25° C., P = 1.8 bar |
| | COS, HCN | |

The knockout water from the fuel combustor (P413F) and the upgrading units are mixed with the knockout from the FT effluent treatment units, the RGS unit, and the Claus plant and sent to the sour stripper (SS; P205) unit that separates sour gas from the water effluent. The distillate rate of the SS is varied such that complete separation between the sour gas and water is achieved. The sour gas is compressed and recycled to the Claus plant, and the water effluent is input either to an electrolyzer unit or to the heat and power recovery network (HEPN). The remaining acid gas from the Rectisol unit (P204) is compressed and preheated to 450° F. before being sent to the Claus furnace splitter (S206). The split fraction is adjusted to maintain a 2:1 molar ratio of H₂S/SO₂ in the inlet to the first sulfur converter (P207). Low-pressure oxygen from the ASU and recycle gas from the sour stripper (P205) are also preheated to 450° F. and sent to the Claus furnace (P206), along with the designated stream from the Claus furnace splitter (S206). The inlet oxygen flow rate is adjusted to provide 1.2 times the stoichiometric requirement for complete combustion. Due to the high temperature present in the furnace, any ammonia present in the feed stream will also be completely decomposed via the following reaction:

4NH₃+3O→2N₂+6H₂O (4)

The furnace effluent is then passed through a series of converter units where the H₂S reacts with SO₂ to form sulfur via then following reaction:

2H₂S+SO₂→2H₂O+3S (5)

The fractional conversions of H₂S are determined such that the inlet stream temperatures of the sulfur separators (P208, P210, P212) are 10° C. higher than the outlet temperatures. This is done to avoid turning the sulfur separators into heat sinks in the heat and energy integration calculation, which are discussed in the second part of this series of papers. All of the sulfur is extracted in these units and mixed in a sulfur pit (M207). The tail gas from P212 is preheated to 450° F. before being sent to a hydrolyzer (P213) to convert any remaining gas-phase sulfur species to H₂S.27 The hydrolyzer effluent is cooled to 35° C., sent to a flash unit (P213F) to knock out water, and compressed to 25 bar before being recycled back to P204.

### Example 1.5

### Hydrocarbon Production (Area P300)

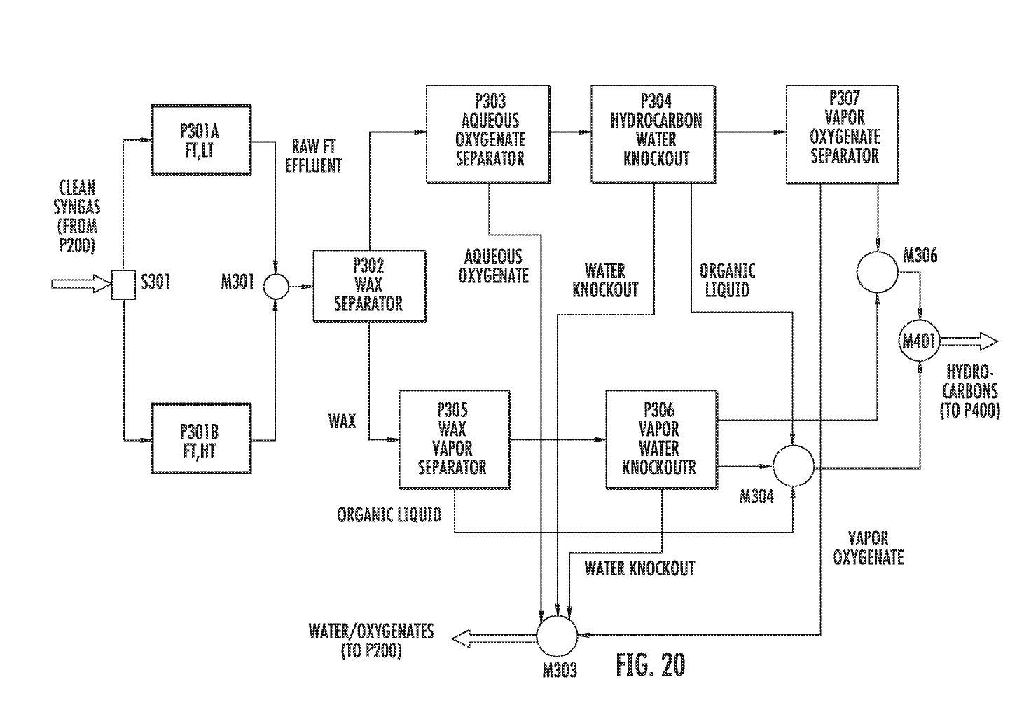

In the third section, clean syngas is converted into a range of hydrocarbon compounds in the FT reactors (FIG. 20) via the generic reaction

nCO+(n−p+0.5m)H₂→CₙHₘOₚ+(n−p)H₂O (6)

where n, m, and p are the number of carbon, hydrogen, and oxygen atoms, respectively, in a given hydrocarbon compound. The distribution of the hydrocarbon products formed in the reactors can be assumed to follow the theoretical Anderson-Schulz-Flory (ASF) distribution, based on the chain growth probability values (eq 7):

Wₙ=n(1−α)²αⁿ⁻¹ (7)

where Wₙ is the mass fraction of the species with carbon number n and R is the chain growth probability. In the modeling of this unit, the selected R values predict the yields of hydrocarbon products.

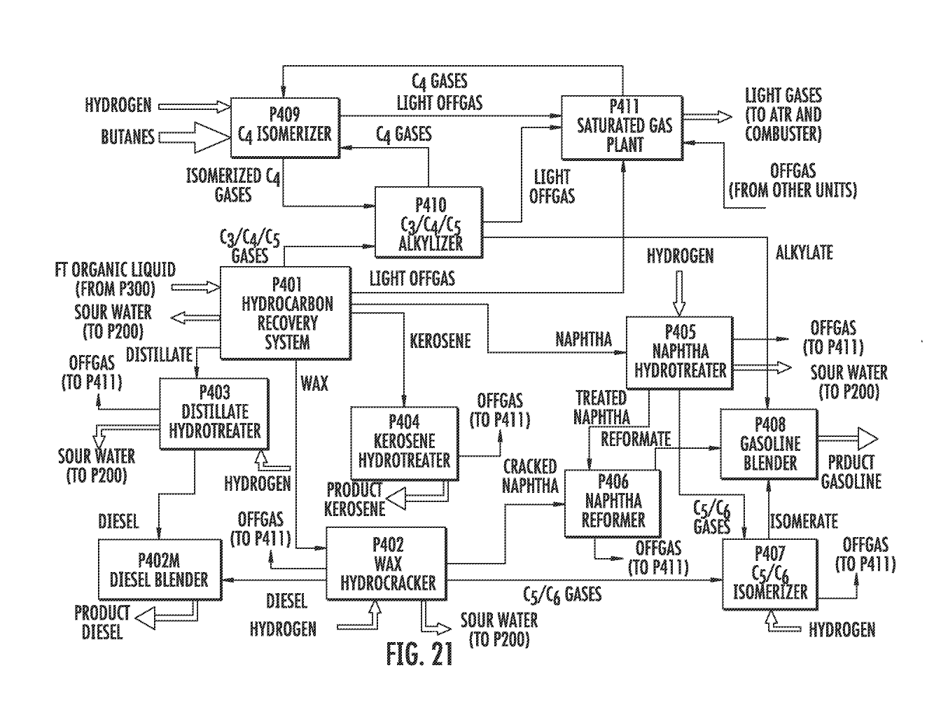

This section consists of two types of FT reactors: one operating at high temperature (P301A, T) 320° C.) and one operating at low temperature (P301B, T) 240° C.). We select the slurry-phase FT reactor system, because of its high conversion from syngas to liquids. The clean syngas from the Rectisol unit is compressed to 24.4 bar and preheated to the corresponding FT operating temperatures. The incoming syngas is split such that the gasoline and diesel product ratio from the upgrading section (FIG. 21) is consistent with the U.S. transportation demand data.

The conversion of CO in each of the FT reactors is assumed to be 80 mol %.11 This high conversion can be achieved in a slurry-phase system, because of the high syngas-catalyst contact and mixing in the reactor. Oxygenated compounds formed in the reactors are represented by vapor phase (eq 8), aqueous phase (eq 9), and organic phase (eq 10) pseudo-components. The total converted carbon present in each pseudo-component is 0.1%, 1.0%, and 0.4%, respectively.

2.43CO+4.275H₂→C_{2.43}H_{5.69}O+1.43H₂O (8)

1.95CO+3.815H₂→C_{1.95}H_{5.77}O_{1.02}+1.93H₂O (9)

4.78CO+9.25H₂→C_{4.78}H_{11.14}O_{1.1}+3.68H₂O (10))

The distribution of the remaining carbon follows a slightly modified ASF distribution that is described in section 4.2, to account for the increased formation of light hydrocarbons. The high-temperature process has a lower chain growth probability (R) 0.65) that favors the formation of gasoline-length hydrocarbons, while the low-temperature process (R) 0.73) forms heavier hydrocarbons and waxes. Hydrocarbon products up to C₂₀ are represented by paraffin and olefin (one double bond) compounds, where the fraction of carbon in the paraffin form is 20% for C₂-C₄, 25% for C₅-C₆, and 30% for C₇-C₂₀.28 C₄-C₆ hydrocarbons are present in both linear and branched form with a branched carbon fraction of 5% for C₄ and 10% for C₅-C₆.28 C₂₁-C₂₉ hydrocarbons are represented by pseudocomponents that have properties consistent with 70 mol % olefin and 30 mol % paraffin. All C₃₀₊ compounds are represented by a generic wax pseudo-component (C_{52.524}H_{105.648}O_{0.335}).

Treatment of the FT effluent streams (FIG. 20) follows from a Bechtel simulation of a detailed product separation and catalyst recovery process (Bechtel, 1998, which is incorporated herein by reference as if fully set forth). The FT effluent streams are mixed and passed through a wax separation unit (P302). The vapor is cooled, sent to an aqueous oxygenate separator (P303), flashed to remove entrained water (P304), and passed through a vapor oxygenate separator (P307). The knocked-out water and oxygenates are sent to the knockout mixer (M303), while the vapor and organic liquids are sent to the first hydrocarbon mixer (M306). The wax from P302 is cooled to 150° C. before being sent to an entrained vapor removal unit (P305). The wax is sent to the second hydrocarbon mixer (M304) and the vapor is further cooled to 40° C. and sent to a flash unit (P306) for water knockout. The vapor is sent to M306, the organic liquid is sent to M304, and the knockout water is sent to M303. All hydrocarbons are directed to M401 before being sent to the upgrading section.

### Example 1.6

### Hydrocarbon Upgrading (Area P400)

The role of the fourth section (FIG. 21) is to upgrade the hydrocarbons to fuel quality. The hydrocarbons are first sent to a hydrocarbon recovery unit (P401), where they are separated into light gases, C₃-C₅ gases, naphtha, kerosene, distillate, wax, and wastewater (Table 6). The wastewater is sent to the sour water mixer, and the light gases are sent to the saturated gas plant (P411). The remaining outlet streams are sent to upgrading units based on a Bechtel design (Bechtel, 1998; Bechtel, 1992, which are incorporated herein by reference as if fully set forth). Since the process operating conditions for each upgrading unit are unknown, the distribution of the outlet for each unit is assumed to be equal to the Bechtel baseline Illinois No. 6 coal case study (Bechtel, 1992, which is incorporated herein by reference as if fully set forth) For each upgrading unit, the percentage of carbon present in the effluent is calculated and the carbon in the inlet is distributed to the effluent in appropriate proportions. When applicable, the hydrogen balance is satisfied by adjusting the input flow rate of upgrading hydrogen sent to the reactor. If hydrogen is not sent directly to a unit, then the atomic balances are satisfied by adjusting the carbon fractions present in the light gas output, so that the difference between the adjusted values and the case study values is minimized using a Euclidean distance metric. Kerosene production is incorporated into the model by assuming that a cut will be taken from the hydrocarbon distillation unit between the liquid naphtha and the distillate such that the ratio of kerosene and diesel output follows the U.S. transportation demand for these fuels. The outlet flash conditions from each upgrading unit, along with the requisite hydrogen to carbon ratio (when applicable), is given in Table 74.

The kerosene and distillate cuts are hydrotreated (P404 and P403, respectively) to remove sour water and form the products kerosene and diesel. The output yield of the light gases from the kerosene hydrotreater is assumed to be the same as the distillate hydrotreater. The naphtha is sent to a hydrotreater (P405) to remove sour water and separate C₅-C₆ gases from the treated naphtha. The wax from P401 is sent to a hydrocracker (P402), where finished diesel product is sent to the diesel blender (P402M), along with the diesel from P403. C₅-C₆ gases from both P402 and P405 are sent to a C₅/C₆ isomerizer. Naphtha from both P402 and P405 is sent to a naphtha reformer (P406).

C₄ isomerization (P409) converts in-plant and purchased butane to isobutane, which is fed into the alkylation unit (P410). Purchased butane is added to the isomerizer such that 80 wt % of the total flow going into the unit is composed of n-butane.29 The isomerized C₄ gases are then mixed with the C₃-C₅ gases from P401 in the C₃/C₄/C₅ alkylation unit (P410), where the C₃-C₅ olefins are converted to high-octane gasoline blending stock. The remaining butane is sent back to P409, while all light gases are mixed with the light gases from the other upgrading units and sent to the saturated gas plant (P411), which uses deethanizer, depropanizer, and debutanizer towers to separate the C₄ gases from the other lights.29 All C₄ gases from P411 are recycled back to the C₄ isomerizer and a cut of C₃ gases are sold as byproduct propane.

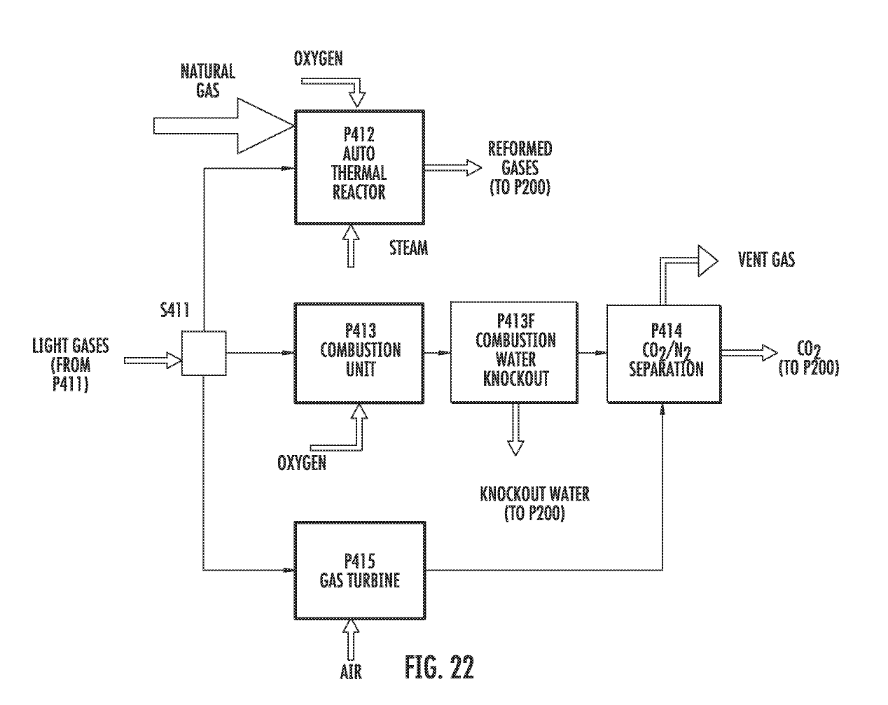

The remaining gases from P411 are divided and sent to either the ATR unit (P412), a combustor (P413), or a gas turbine engine (P415) before being recycled back to the RGS unit (FIG. 22). The fraction going to the combustor unit (T) 1300° C.) is first compressed and then mixed with oxygen (1.2 times the stoichiometric amount). The flow rate to P413 is adjusted to satisfy the plant fuel requirement of the CBGTL process. The effluent is then cooled to 35° C., flashed (P413F), and sent to a single-stage Rectisol unit (P414), where the CO₂ is separated from the inert N₂. Split fractions of the CO₂ are equivalent to those given in Table 5. The N₂ stream is purged while the recovered CO₂ is mixed with the recovered CO₂ from P204 and recycled to the RGS unit. The hydrocarbons going to the ATR are compressed and preheated to 800° C. before entering the unit. Natural gas (Table 3) is added along with 35 bar of saturated steam, such that the input mole ratio of H2O to carbon is 0.5:1. Oxygen is added to keep a net-zero heat duty value, and the oxygen and steam inputs are also preheated to the unit's operating temperature.

Alternatively, the light gases can pass through a gas turbine engine instead of the ATR to produce electricity for the plant (FIG. 22). Note that, in the gas turbine process alternative, the ATR will still exist to reform the natural gas feedstock. The operation of the gas turbine is modeled by a series of compressors, combuster reactor, and turbines as follows. The light gases are compressed and heated to 467.5 psia and 385° F. before they are mixed with pressurized CO₂ from the recycle stream in the syngas cleaning section (FIG. 19) and sent to the gas turbine combuster (P415). The role of this CO₂ stream is to dilute the calorific value of the gas turbine feed stream and minimize the production of NOₓ in the gas turbine combuster. To supply the oxygen requirement for combustion (1.1 times the stoichiometric amount), compressed air is cofed into the combuster unit from an air compression train. This train consists of a compressor with 87% polytropic efficiency (98.65% mechanical efficiency) and a splitter to model the 0.1% air leakage and 5.161% cooling flow bypass that will be fed into the gas turbine engine. The gas turbine combuster (P415) operates at 1370° C. with 0.5% heat loss, and its effluents pass through a first gas turbine with 89.769% isentropic efficiency and 98.65% mechanical efficiency. The cooling flow bypass stream is injected into the gas turbine at this point to reduce the exhaust temperature and the entire stream is passed through a second turbine with an exhaust pressure of 1.065 bar. Gas turbine effluents are cooled to 35° C. and flashed to remove any liquid water in the stream. They are compressed to 27.3 bar and cooled once again before entering the single-stage Rectisol unit for CO₂ separation. Finally, the ATR and gas turbine effluent are sent back to the RGS unit.

### Example 1.7

### Oxygen and Hydrogen Production (Area P500)

The oxygen and hydrogen production section (FIG. 23) consists of alternative technologies that are presently available or expected to be in commercial status in the future. Considered alternatives include (i) an ASU that produces a 99.5 wt % O₂ stream and hydrogen purchase from steam reforming of natural gas, or (ii) an electrolyzer unit that produces pure H₂ and O₂ from the plant's water effluent and electricity. Electricity can be obtained from the grid or alternative sources such as solar, wind, and nuclear technologies as they become more available in the future.

If hydrogen is produced off-site, the oxygen input must be obtained from an ASU. Air is initially compressed from ambient conditions to 190 psia and then sent to the ASU (P501), where a 99.5 wt % O₂ stream (T=90° F., P=125 psia) is recovered and the nitrogen-rich stream (T=70° F., P=16.4 psia) is vented. A portion of the oxygen stream is split and fed into the low-pressure Claus furnace, while the balance is compressed to 32 bar for use with the remaining process units. Hydrogen is purchased from steam reforming of methane (SRM) technology, such that its total provides the required hydrogen for the RGS unit and the upgrading units. If hydrogen is produced on-site, an electrolyzer unit will be utilized to produce pure H₂ and O₂ from the water effluent of the SS unit.7 Oxygen that is not consumed by the CBGTL process will be sold as a byproduct.

### Example 1.8

### Heat and Power Recovery (Area P600)

The heat and power recovery system utilizes heat engines and pumps that interact with process streams to produce steam or electricity. Plant water and additional purchased water are used to produce steam required by the various process units. The full description and mathematical models of the heat and power integration step are detailed in Example 2, which outlines a three-stage decomposition framework consisting of the minimization of hot/cold/power utility requirement, the minimization of heat exchanger units, and the minimization of the annualized cost of heat exchange. Once the full heat and power integration step is completed, the obtained costs are factored into the economic analysis of the entire process, as described in Examples 1.22-1.25.

### Example 1.9

### Process Modeling

Although most of the operating units in the CBGTL process are modeled using standard Aspen Plus modules (as otherwise described herein), the gasifiers, FT units, and all upgrading units are modeled using the USER2 block option. The USER2 block allows the Aspen Plus engine to dynamically link to a Microsoft Excel spreadsheet, where user-input calculations can provide the necessary effluent concentrations. The outlet stream conditions of the USER2 blocks can then be set to a given temperature and pressure, based upon predefined values. The USER2 blocks serve as a means of implementing (i) a novel stoichiometric model for biomass and coal gasification, (ii) a probabilistic FT model based on the chain growth factor (α), and (iii) individual models for the upgrading units based on a Bechtel design. The following section details the mathematical models designed for the CBGTL process.

### Example 1.10

### Coal/Biomass Gasification

The reaction system within a gasifier consists of a series of pyrolysis, combustion, and gasification steps that are designed to release the volatile matter within the solid feedstock and subsequently convert the residual solid to syngas. Though it has been documented that the major gas phase components (H₂O, H₂, CO, CO₂) will be close to thermodynamic equilibrium via the water gas shift (WGS) reaction (eq 1), the residual gases (C₁-C₂ Hydrocarbons, H₂S, COS, NH₃, HCN, HCl, etc.) will often be present in concentrations far above their equilibrium values. A detailed model of the kinetics within a gasifier can be a challenging task, especially since the accuracy of the model will be strongly dependent on the choice of rate constants for the multiple reactions within the unit. Several models have been developed using appropriate conditions for entrained flow and circulating flow gasifiers. A novel stoichiometric gasifier model capable of determining the effluent flow rates based on a variety of experimental data is disclosed herein.

### Example 1.11

### Biomass Pyrolysis

Prior to gasification of the residual solids, the volatile compounds are released via the pyrolysis reactions. The derivation of an overall pyrolysis reaction for biomass or coal depends on multiple factors, including (i) heating rate, (ii) final temperature, (iii) residence time, (iv) particle size, (v) gasifier pressure, and (vi) gasifier type. An approximate mechanism will give some insight into the initial composition of light hydrocarbons and can provide more accurate effluent flow rates for the nonequilibrium components. Detailed calculation of the stoichiometric pyrolysis coefficients for the individual biomass components hemicellulose (eq 11), cellulose (eq 12), Lig-C (eq 13), Lig-H (eq 14), and Lig-O (eq 15) are presented below.

C₅H₈O₄→2.2C₍ₛ₎+1.898H₂+0.71CO+0.525CH₄+1.284CO₂+0.092C₂H₄+0.049C₂H₆+0.722H₂O (11)

C₆H₁₀O₅→0.877C₍ₛ₎+0.889H₂+2.163CO+1.488CH₄+1.067CO₂+0.175C₂H₄+0.028C₂H₆+0.703H₂O (12)

C₁₅H₁₄O₄→9.675C₍ₛ₎+3.68H₂+1.95CO+0.403CO₂+0.234CH₄+1.136C₂H₂+0.234C₂H₄+1.24H₂O (13)

C₂₂H₂₈O₉→11C₍ₛ₎+5.507H₂+4.9CO+1.05CO₂+1.443CH₄+1.804C₂H₄+2H₂O (14)

C₂₀H₂₂O₁₀→11C₍ₛ₎+5.721H₂+4.9CO+1.55CO₂+0.729CH₄+0.911C₂H₄+2H₂O (15)

The assumptions for the biomass pyrolysis coefficient calculations are presented below.

A1. Biomass compositions are reported on a dry, ash-free (daf) basis.
A2. Char will be explicitly modeled as solid carbon (C(s)).
A3. Tar output will not be considered, because it is assumed that all tar formed will be reformed via O₂ or H₂O within the gasifier.
A4. All products of the pyrolysis reaction will consist of the following compounds: H₂O, H₂, CO, CO₂, CH₄, C₂H₂, C₂H₄, C₂H₆, and char.
A5. The main constituents of biomass are cellulose, hemicellulose, Lig-C, Lig-O, and Lig-H, which are represented as C₆H₁₀O₅, C₅H₈O₄, C₁₅H₁₄O₄, C₂₀H₂₂O₁₀, and C₂₂H₂₈O₉, respectively.
A6. An independent pyrolysis equation will occur for each biomass monomer.
A7. The initial composition of volatiles of hemicellulose and cellulose decomposition will follow from Table 2 of Yang et al, 2007, which is incorporated herein by reference as if filly set forth. The residual char will also be based on the observations in Yang et al, 2007, which is incorporated herein by reference as if fully set forth.
A8. All unaccounted carbon, hydrogen, and oxygen in the mass balance for the decomposition from assumption A7 is assumed to be present in H₂O, CH₄, C₂H₄, and CO for cellulose and in H₂O, CH₄, C₂H₄, and H₂ for hemicellulose.
A9. Since Yang et al., 2007 do not provide a decomposition framework for each lignin monomer, one will be adapted from the kinetic model in Table 3 of Ranzi et al., 2008, which is incorporated herein by reference as if fully set forth, by assuming that all reactions present in the kinetic model proceed to completion.
A10. All unaccounted oxygen in the mass balance for the decomposition from assumption A9 is assumed to be present in CO₂. All unaccounted carbon and hydrogen in the mass balance is assumed to be present as tar, which will decompose into CH₄, C₂H₂, and C₂H₄ such that CH₄ and C₂H₄ are present in the same proportions as in the initial volatiles composition. All residual unaccounted hydrogen is assumed to be present as H₂.

The dry composition of the vapor phase for cellulose and hemicellulose pyrolysis is given in Table 2 of Yang et al., 2007, which is incorporated herein by reference as if fully set forth and is reproduced in Table 7.

TABLE 7
Dry Composition of the Vapor Phase
for Cellulose and Hemicellulose Pyrolysis^{a}

| | Gas Product Yield (mmol/g-biomass ar) | | | | | |
|---|---|---|---|---|---|---|
| sample | H₂ | CO | CH₄ | CO₂ | C₂H₄ | C₂H₆ |
| hemicellulose | 8.75 | 5.37 | 1.57 | 9.72 | 0.05 | 0.37 |
| cellulose | 5.48 | 9.91 | 1.84 | 6.58 | 0.08 | 0.17 |
| lignin | 20.84 | 8.46 | 3.98 | 7.81 | 0.03 | 0.42 |

The yields of gas products are normalized to the as-received (ar) weight of biomass. Furthermore, it is also noted that the weight percentage of char remaining after pyrolysis is ˜6.5% for cellulose and ˜20% for hemicellulose. It is assumed that the cellulose is of the form C₆H₁₀O₅ and the hemicellulose is of the form C₅H₈O₄. Thus, 1 g is equivalent to 6.167 mmol for cellulose and 7.568 mmol for hemicellulose. Furthermore, the molar amount of char remaining is 5.412 mmol for cellulose and 16.653 mmol for hemicellulose. We now have

6.167C₆H₁₀O₅→5.412C₍ₛ₎+5.48H₂+9.91CO+1.84CH₄+6.58CO₂+0.08C₂H₄+0.17C₂H₆+C_{12.763}H_{42.015}O_{7.767} (16)

7.568C₅H₈O₄→16.653C₍ₛ₎+8.75H₂+5.37CO+1.57CH₄+9.72CO₂+0.05C₂H₄+0.37C₂H₆+C_{3.689}H_{34.346}O_{5.463} (17)

It is assumed that C_{12.763}H_{42.015}O_{7.767} will completely degrade into H₂O, CH₄, C₂H₄, and CO, and C_{3.689}H_{34.346}O_{5.463} will degrage into H₂O, CH₄, C₂H₄, and H₂ for cellulose and hemicellulose, respectively. The relative ratio of CH₄ to C₂H₄ is estimated using the relative ratio of CH₄ to the C₂ Hydrocarbons in Table 7. That is, it can be assumed that CH₄:C₂H₄ is equal to 7.36 for cellulose and 3.738 for hemicellulose. The decomposition reactions are then given by

C_{12.763}H_{42.015}O_{7.767}→4.337H₂O+7.338CH₄+0.997C₂H₄+3.431CO (18)

C_{3.689}H_{34.346}O_{5.463}→5.463H₂O+2.403CH₄+0.643C₂H₄+5.618H₂ (19)

After normalizing for one mole of input biomass monomer, the following equations arise:

C₆H₁₀O₅→0.877C₍ₛ₎+0.889H₂+2.163CO+1.488CH₄+1.067CO₂+0.175C₂H₄+0.028C₂H₆+0.703H₂O

C₅H₈O₄→2.2C₍ₛ₎+1.898H₂+0.71CO+0.525CH₄+1.284CO₂+0.092C₂H₄+0.049C₂H₆+0.722H₂O

Note that the lignin decomposition provided by Table 7 does not specifically refer to Lig-C, Lig-H, or Lig-O. To derive the appropriate lignin pyrolysis equations, we utilize the kinetic model outlined in Table 3 from Ranzi et al., 2008, which is incorporated herein by reference as if fully set forth. The list of reactions for the lumped kinetic model is provided below:

Lig-C→0.35Lig_{CC}+0.1pCourmaryl+0.08Phenol+1.49H₂+H₂O+1.32G{COH₂}+7.05C (20)

Lig-H→Lig_{OH}+C₃H₆O (21)

Lig-O→Lig_{OH}+CCO₂ (22)

Lig_{CC}→0.3pCourmayl+0.2Phenol+0.35C₃H₄O₂+1.2H₂+0.7H₂O+0.25CH₄+0.25C₂H₄+1.3G{COH₂}+0.5G{CO}+7.5C (23)

Lig_{OH}→Lig+0.5H₂+H₂O+CH₃OH+G{CO}+1.5G{COH₂}+5C (24)

Lig→C₁₁H₁₂O₄ (25)

Lig→0.7H₂+H₂O+0.4CH₂O+0.5CO+0.4CH₃OH+0.2CH₃CHO+0.2C₃H₆O₂+0.4CH₄+0.5C₂H₄+G{CO}+0.5G{COH₂}+6C (26)

G{CO₂}→CO₂ (27)

G{CO}→CO (28)

G{COH₂}→CO+H₂ (29)

where Lig-C, Lig-H, and Lig-O are represented as C₁₅H₁₄O₄, C₂₂H₂₈O₉, and C₂₀H₂₂O₁₀, respectively.

It is assumed that (i) all reactions proceed to completion and (ii) the reaction of Lig f→C₁₁H₁₂O₄ is negligible, with respect to the decomposition of Lig. Note that assumption (ii) is justified because the rate of reaction of Lig decomposition is ˜400 times greater at 500 K. Given these assumptions, Lig-C, Lig-H, and Lig-O decomposition reactions are modeled as follows:

C₁₅H₁₄O₄→9.675C₍ₛ₎+3.685H₂+1.95CO+0.0875CH₄+0.0875C₂H₄+1.245H₂O+C_{3.1125}H_{3.44}O_{0.805} (30)

C₂₂H₂₈O₉→11C₍ₛ₎+3.6H₂+4.9CO+0.4CH₄+0.5C₂H₄+2H₂O+C_{4.7}H_{13.2}O_{2.1} (31)

C₂₀H₂₂O₁₀→11C₍ₛ₎+3.6H₂+4.9CO+0.4CH₄+CO₂+0.5C₂H₄+2H₂O+C_{1.7}H_{7.2}O_{1.1} (32)

where all carbon, hydrogen, and oxygen present in pCourmayl, phenol, C₃H₆O, C₃H₄O₂, CH₃OH, and CH₃CHO have been lumped into model C/H/O compounds and COH₂ is assumed to decompose to CO and H₂.

The model C_{3.1125}H_{3.44}O_{0.805} compound is assumed to decompose to CO₂, CH₄, C₂H₂, and C₂H₄, while the model C_{4.7}H_{13.2}O_{2.1} and C_{1.7}H_{7.2}O_{1.1} compounds are assumed to decompose to CO₂, CH₄, C₂H₄, and H₂. C₂H₂ is chosen as a model decomposition compound for Lig-C, because of the high carbon content of C_{3.1125}H_{3.44}O_{0.805}. Similarly, H₂ is chosen as a model decomposition compound for Lig-H and Lig-O due to the high hydrogen content of C_{4.7}H_{13.2}O_{2.1} and C_{1.7}H_{7.2}O_{1.1}, respectively. The ratio of the CH₄ to C₂H₄ in the model compound decomposition is assumed to be equivalent to the ratio of CH₄ to C₂H₄ present after monomer decomposition. That is, CH₄:C₂H₄ is equal to 1 for Lig-C, 0.8 for Lig-H, and 0.8 for Lig-O. The model compound decomposition then takes the form

C_{3.1125}H_{3.44}O_{0.805}→0.403CO₂+0.146CH₄+0.146C₂H₄+1.136C₂H₂ (33)

C_{4.7}H_{13.2}O_{2.1}→1.05CO₂+1.907H₄+1.043CH₄+1.304C₂H₄ (34)

C_{1.7}H_{7.2}O_{1.1}→0.55CO₂+2.12H₂+0.329CH₄+0.411C₂H₄ (35)

Grouping the above equations, the representative equations for the pyrolysis of lignin become

C₁₅H₁₄O₄→9.675C₍ₛ₎+3.685H₂+1.95CO+0.234CH₄+0.403CO₂+0.234C₂H₄+1.136C₂H₂+1.245H₂O

C₂₂H₂₈O₉→11C₍ₛ₎+5.507H₂+4.9CO+1.433CH₄+1.05CO₂+1.804C₂H₄+2H₂O

C₂₀H₂₂O₁₀→11C₍ₛ₎+5.721H₂+4.9CO+0.729CH₄+1.55CO₂+0.911C₂H₄+2H₂O

### Example 1.12

### Biomass Monomer Calculation

The biomass input is characterized by its proximate and ultimate analysis. The proximate analysis details (i) the moisture content, (ii) the ash content, (iii) the volatile content (when heated to ˜1125 K), (iv) the fixed carbon content remaining after heating, and (v) the higher heating value (HHV). The ultimate analysis reports the weight fractions of carbon, hydrogen, oxygen, nitrogen, sulfur, and chlorine of the dry, ash-free biomass. To utilize the above pyrolysis reactions, the compositions of the biomass monomers must be determined from the given proximate and ultimate analysis. Therefore, we formulate a model to approximate the monomer composition such that it most closely resembles the reported analyses.

Indices/Sets/Parameters.

The indices used are

α: Atom index
s: Species index
The sets of all atoms (As_{Biomass}) and species (S_{Biomass}) for the biomass monomer calculation are:

a∈A_{Biomass}={C,H,O}

s∈S_{Biomass}={C₅H₈O₄,C₆H₁₀O₅,C₁₅H₁₄O₄,C₂₀H₂₂O₁₀,C₂₂H₂₈O₉}

The parameters in the monomer model are as follows:

W_{α,Biomass}: weight fraction of atom α in the biomass ultimate analysis
W_{α,s}: weight fraction of atom a in species s
W_{Char,s}: weight fraction of char after pyrolysis of species s

W_{Char,Biomass}: weight fraction of fixed carbon in the biomass proximate analysis

Variables. Continuous variables are used to model the monomer weight fractions. To allow for the possibility that the monomer composition will not match the ultimate and proximate analyses exactly, slack variables are introduced. These variables are given by

Wₛ, Biomass: weight fraction of species s in the biomass
Sₐ: slack variable for atom a mass balance
S_{Char}: slack variable for fixed carbon balance

Constraints. All variables are restricted to be non-negative as in eqs 36-38:

w_{s,Biomass}≧0∀sγS_{Biomass} (36)

sₐ≧0∀a∈A_{Biomass} (37)

s_{Char}≧0 (38)

The weight fractions of monomers must sum to 1, as represented by eq 39:

$${\sum }_{s ∈ {S}_{Biomass}} {w}_{s , Biomass} = 1 \qquad ( 39 )$$

The monomers must also satisfy the mass balances given in the ultimate analysis, within some slack tolerance, as given by eqs 40 and 41:

$$\begin{matrix}{\sum }_{s ∈ {S}_{Biomass}} {w}_{s , Biomass} {w}_{a , s} - {w}_{a , Biomass} \leq {s}_{a} ∀ a ∈ {A}_{Biomass} & ( 40 ) \\ {\sum }_{s ∈ {S}_{Biomass}} {w}_{s , Biomass} {w}_{a , s} - {w}_{a , Biomass} \geq - {s}_{a} ∀ a ∈ {A}_{Biomass} & ( 41 )\end{matrix}$$

A fixed carbon mass balance based on the monomer pyrolysis equations is established as given by eqs 42 and 43:

$$\begin{matrix}{\sum }_{s ∈ {S}_{Biomass}} {w}_{s , Biomass} {w}_{Char , s} - {w}_{Char , Biomass} \leq {s}_{Char} & ( 42 ) \\ {\sum }_{s ∈ {S}_{Biomass}} {w}_{s , Biomass} {w}_{Char , s} - {w}_{Char , Biomass} \geq - {s}_{Char} & ( 43 )\end{matrix}$$

Objective Function. By minimizing the slack variables (eq 44), the ultimate and proximate analyses can be approximated as closely as possible.

$${min}_{{s}_{a} , {s}_{Char}} {\sum }_{a ∈ {A}_{Biomass}} {λ}_{a} {s}_{a} + {s}_{Char} \qquad ( 44 )$$

where λₐ g 1 is introduced to emphasize the importance of satisfying the atom balances, compared to the fixed carbon balance. For this analysis, λₐ is set to 100 for all a.

### Example 1.13

### Results

The biomass used in the CBGTL process is herbaceous switchgrass. Using the ultimate analysis given in Table 2, the parameters in Table 8 are calculated.

TABLE 8
Parameters of the Biomass Monomer Calculation

| | |
|---|---|
| w_{C,Biomass} = 0.50576 | w_{C,C}_{₁₅}_{H}_{₁₄}_{O}_{₄} = 0.69751 |
| w_{H,Biomass} = 0.06160 | w_{H,C}_{₁₅}_{H}_{₁₄}_{O}_{₄} = 0.05463 |
| w_{O,Biomass} = 0.43263 | w_{O,C}_{₁₅}_{H}_{₁₄}_{O}_{₄} = 0.24777 |
| w_{Char,Biomass} = 0.22716 | w_{Char,C}_{₁₅}_{H}_{₁₄}_{O}_{₄} = 0.44993 |
| w_{C,C}_{₅}_{H}_{₈}_{O}_{₄} = 0.45450 | w_{C,C}_{₂₂}_{H}_{₂₈}_{O}_{₉} = 0.60535 |
| w_{H,C}_{₅}_{H}_{₈}_{O}_{₄} = 0.06103 | w_{H,C}_{₂₂}_{H}_{₂₈}_{O}_{₉} = 0.06466 |
| w_{O,C}_{₅}_{H}_{₈}_{O}_{₄} = 0.48435 | w_{O,C}_{₂₂}_{H}_{₂₈}_{O}_{₉} = 0.32988 |
| w_{Char,C}_{₅}_{H}_{₈}_{O}_{₄} = 0.20000 | w_{Char,C}_{₂₂}_{H}_{₂₈}_{O}_{₉} = 0.30271 |
| w_{C,C}_{₆}_{H}_{₁₀}_{O}_{₅} = 0.44440 | w_{C,C}_{₂₀}_{H}_{₂₂}_{O}_{₁₀} = 0.56866 |
| w_{H,C}_{₆}_{H}_{₁₀}_{O}_{₅} = 0.06216 | w_{H,C}_{₂₀}_{H}_{₂₂}_{O}_{₁₀} = 0.05249 |
| w_{O,C}_{₆}_{H}_{₁₀}_{O}_{₅} = 0.49332 | w_{O,C}_{₂₀}_{H}_{₂₂}_{O}_{₁₀} = 0.37876 |
| w_{Char,C}_{₆}_{H}_{₁₀}_{O}_{₅} = 0.00650 | w_{Char,C}_{₂₀}_{H}_{₂₂}_{O}_{₁₀} = 0.31279 |

Optimization of the biomass monomer model (eqs 36-44) yields the biomass composition, W_{s,Biomass}, which is presented in Table 9.

TABLE 9
Biomass Composition from Monomer Calculation

w_{C}_{₅}_{H}_{₈}_{O}_{₄}_{,Biomass} = 0.1725
w_{C}_{₆}_{H}_{₁₀}_{O}_{₅}_{,Biomass} = 0.5103
w_{C}_{₁₅}_{H}_{₁₄}_{O}_{₄}_{,Biomass} = 0.0566
w_{C}_{₂₂}_{H}_{₂₈}_{O}_{₉}_{,Biomass} = 0.2588
w_{C}_{₂₀}_{H}_{₂₂}_{O}_{₁₀}_{,Biomass} = 0.0017

Using these weight fractions and the corresponding pyrolysis equations (eqs 11-15), the overall chemical formula for the CBGTL feedstock biomass is C_{7.33}H_{10.675}O_{4.706} and the overall biomass pyrolysis equation is

C_{7.33}H_{10.675}O_{4.706}→3.1553C₍ₛ₎+0.8715H₂O+2.154H₂+1.4618CO+1.1862CO₂+0.7875CH₄+0.0434C₂H₂+0.2898C₂H₄+0.0380C₂H₆ (45)

Note that all N, S, and Cl atoms are assumed to pyrolyze as NH₃, H₂S, and HCl, respectively.

### Example 1.14

### Coal Pyrolysis

From the ultimate analysis of coal on a dry, ash-free (daf) basis, the chemical formula for Illinois No. 6 coal, as used in the CBGTL process, is calculated to be C_{6.687}H_{5.387}O_{0.566}N_{0.113}S_{0.113}. Coal proximate analysis (daf) is used to determine the molar amount of carbon that goes into char while the rest of the elemental components goes into volatile matters. Table 10 breaks down the elemental distribution in coal, char, and volatile matters.

TABLE 10
Elemental Composition in Coal, Char, and Volatile Matters

| | wt % (daf) | moles of C in char |
|---|---|---|
| fixed carbon | 52.288 | 4.353 |
| volatile matter | 47.712 | |

Elemental Analysis

| element | coal (mol) | char (mol) | volatile matter (mol) |
|---|---|---|---|
| C | 6.687 | 4.353 | 2.334 |
| H | 5.387 | | 5.387 |
| O | 0.566 | | 0.566 |
| N | 0.113 | | 0.113 |
| S | 0.113 | | 0.113 |

Elemental compositions of volatile matters in Table 10 are converted into the following components: C₍ₛ₎, CO, CO₂, H₂, H₂O, CH₄, N₂, H₂S, NH₃, HCN, Ar, and HCl. The following subsections outline the mathematical model that gives the overall coal pyrolysis reaction.

Sets. The set of all atoms A_{Pyr,coal} is defined as

a∈A_{Pyr,coal}={Ar,C,H,O,N,S,Cl}

The set of all gaseous species produced from the pyrolysis step is given as follows:

s∈S_{Pyr,coal}={C₍ₛ₎,CO,CO₂,H₂,H₂O,CH₄,N₂,H₂S,NH₃,HCN,HCl,Ar}

A new index, called ratio, is now defined that represents the relationship between certain species involved in the coal pyrolysis process. The set Ratio contains these specific relationships as denoted below:

Ratio={ratio₁,ratio₂,ratio₃}

where ratio₁ represents CO:CO₂, ratio₂ represents CO₂:CH₄, and ratio₃ represents CH₄:other components in the pyrolysis gaseous products.

Parameters. The following parameters are defined:

W_{a,coal}: weight fraction of atom a in daf coal sample
AWₐ: atomic weight of atom a
FCₐ: fixed carbon weight fraction in daf coal sample
E_{a,s}: number of a atoms in species s

The composition of the pyrolysis products varies depending on the gasifier type, coal composition, and other factors, as mentioned previously. Since laboratory data of the various types of coal are not readily available, typical devolatilization data such as those given in Table 11 can be used to predict the stoichiometric coefficients of pyrolysis products. Note that the values in Table 11 do not distinguish between coal types and do not require detailed information about the ultimate analysis and devolatilization products of each individual coal. Several correlations have been developed to predict the gas compositions of pyrolysis products. However, when applied to the various coal data used for the parameter estimation of the gasifier model, the correlations do not consistently close the atomic balance of each coal type. Thus, the generic data in Table 11 are used to calculate the pyrolysis reaction.

TABLE 11
Typical Coal Devolatilization Data^{a}

| distribution of coal gas | % (v/v) |
|---|---|
| CO₂ | 6.1 |
| CO | 20.6 |
| H₂ | 13.1 |
| CH₄ | 50.3 |
| other (hydrocarbons, H₂S, N₂) | 9.9 |

Variables. The following variables are defined to model the coal pyrolysis reaction. Continuous variables are used to model the species molar flow rates from the pyrolysis reaction. To allow for the possibility that the species composition will not exactly match the data in Table 11, slack variables are introduced.

Nₛ: molar flow rate of species s
sᵣₐₜᵢₒ: slack variable for species ratio constraints, where ratio∈Ratio

Constraints. The equations that give the stoichiometric coefficients of the coal pyrolysis reaction are the following. Equations 46 and 47 model the atomic balances during coal pyrolysis:

$$\begin{matrix}{\dot{M}}_{Coal} ( \frac{{w}_{a , Coal}}{{AW}_{a}} - \frac{{FC}_{a}}{{AW}_{a}} ) = {\sum }_{s ∈ {S}_{Pyr , coal}} {E}_{a , s} {\dot{N}}_{s} \\ a = C & ( 46 ) \\ {\dot{M}}_{Coal} ( \frac{{w}_{a , Coal}}{{AW}_{a}} ) = {\sum }_{s ∈ {S}_{Pyr , coal}} {E}_{a , s} {\dot{N}}_{s} \\ a ∈ {A}_{Pyr , coal} \backslash \{ C \} & ( 47 )\end{matrix}$$

where M_{Coal} is the mass flow rate of coal. All atoms are assumed to be converted to volatile species (eq 47), with the exception of carbon. To determine the amount of carbon that remains as char, the fixed carbon weight fraction is first subtracted from
the weight fraction of carbon. All the Cl atoms from coal are associated with HCl, and all the S atoms are associated with H₂S.

For the conversion of N atoms in the coal pyrolysis process, it has been documented that the major nitrogenous products are N₂, HCN, and NH₃. The HCN and NH₃ yields increase with temperature. At high temperature (1300° C.), the HCN/NH₃ ratio is ˜1. N₂ continues to be the dominant nitrogenous gas product (up to 40% yield at 1100° C., where yield signifies the mass percentage of elemental nitrogen in total coal nitrogen). Based on these results, it is assumed that (i) 40% of the nitrogen in coal goes to N₂, and (ii) the HCN/NH₃ ratio is equal to 1 at a coal gasifier temperature of 1427° C. (see eqs 48 and 49).

$$\begin{matrix}0.4 {\dot{M}}_{Coal} \frac{{w}_{a , Coal}}{{AW}_{a}} = {\sum }_{s = {N}_{2}} {E}_{a , s} {\dot{N}}_{s} \\ a = N & ( 48 ) \\ {\dot{N}}_{{NH}_{3}} = {\dot{N}}_{HCN} & ( 49 )\end{matrix}$$

Additional constraints are added based on the expected yields of the coal pyrolysis reactions. The following three constraints utilize information from Table 11 to constrain the ratio of CO:CO₂, CO₂:CH₄, and CH₄:other products. The H₂ amount is left to be determined via the atomic balance.

$$\begin{matrix}\frac{{\dot{N}}_{CO}}{{\dot{N}}_{{CO}_{2}}} - \frac{{V}_{CO}}{{V}_{{CO}_{2}}} \leq {s}_{{ratio}_{1}} & ( 50 ) \\ \frac{{\dot{N}}_{CO}}{{\dot{N}}_{{CO}_{2}}} - \frac{{V}_{CO}}{{V}_{{CO}_{2}}} \geq - {s}_{{ratio}_{1}} & ( 51 ) \\ \frac{{\dot{N}}_{{CO}_{2}}}{{\dot{N}}_{{CH}_{4}}} - \frac{{V}_{{CO}_{2}}}{{V}_{{CH}_{4}}} \leq {s}_{{ratio}_{2}} & ( 52 ) \\ \frac{{\dot{N}}_{{CO}_{2}}}{{\dot{N}}_{{CH}_{4}}} - \frac{{V}_{{CO}_{2}}}{{V}_{{CH}_{4}}} \geq {s}_{{ratio}_{2}} & ( 53 ) \\ \frac{{\dot{N}}_{{CH}_{4}}}{{\sum }_{s = {N}_{2} , {NH}_{3} , HCN , {H}_{2} S , HCl} {\dot{N}}_{s}} - \frac{{V}_{{CH}_{4}}}{{V}_{others}} \leq {s}_{{ratio}_{3}} & ( 54 ) \\ \frac{{\dot{N}}_{{CH}_{4}}}{{\sum }_{s = {N}_{2} , {NH}_{3} , HCN , {H}_{2} S , HCl} {\dot{N}}_{s}} - \frac{{V}_{{CH}_{4}}}{{V}_{others}} \geq - {s}_{{ratio}_{3}} & ( 55 )\end{matrix}$$

where V is the volumetric distribution given in Table 11.

The variables Nₛ and sᵣₐₜᵢₒ are constrained to take positive values:

\dot{N}ₛ≧0∀s∈S_{Pyr,Coal(}56)

sᵣₐₜᵢₒ≧0∀ratio∈Ratio (57)

Objective Function. The composition yield of the pyrolysis reaction can be estimated by minimizing the slack variables as follows:

$${min}_{{\dot{N}}_{s} , {s}_{ratio}} {\sum }_{ratio} {s}_{ratio} \qquad ( 58 )$$

Optimization Model. The proposed model is a nonlinear optimization (NLP) model and takes the following form:

$${min}_{{s}_{ratio}} {\sum }_{ratio} {s}_{ratio}$$

$$subject to$$

$${\dot{M}}_{Coal} ( \frac{{w}_{a , Coal}}{{AW}_{a}} - \frac{{FC}_{a}}{{AW}_{a}} ) = {\sum }_{s ∈ {S}_{Pyr , Coal}} {E}_{a , s} {\dot{N}}_{s}$$

$$a = C$$

$${\dot{M}}_{Coal} ( \frac{{w}_{a , Coal}}{{AW}_{a}} ) = {\sum }_{s ∈ {S}_{Pyr , Coal}} {E}_{a , s} {\dot{N}}_{s}$$

$$a ∈ {A}_{Pyr , Coal} \backslash \{ C \}$$

$$0.4 {\dot{M}}_{Coal} ( \frac{{w}_{a , Coal}}{{AW}_{a}} ) = {\sum }_{s = {N}_{2}} {E}_{a , s} {\dot{N}}_{s}$$

$$a = N$$

$${\dot{N}}_{{NH}_{3}} = {\dot{N}}_{HCN}$$

$$\frac{{\dot{N}}_{CO}}{{\dot{N}}_{{CO}_{2}}} - \frac{{V}_{CO}}{{V}_{{CO}_{2}}} \leq {s}_{{ratio}_{1}}$$

$$\frac{{\dot{N}}_{CO}}{{\dot{N}}_{{CO}_{2}}} - \frac{{V}_{CO}}{{V}_{{CO}_{2}}} \geq - {s}_{{ratio}_{1}}$$

$$\frac{{\dot{N}}_{{CO}_{2}}}{{\dot{N}}_{{CH}_{4}}} - \frac{{V}_{{CO}_{2}}}{{V}_{{CH}_{4}}} \leq {s}_{{ratio}_{2}}$$

$$\frac{{\dot{N}}_{{CO}_{2}}}{{\dot{N}}_{{CH}_{4}}} - \frac{{V}_{{CO}_{2}}}{{V}_{{CH}_{4}}} \geq - {s}_{{ratio}_{2}}$$

$$\frac{{\dot{N}}_{{CH}_{4}}}{{\sum }_{s = {N}_{2} , {NH}_{3} , HCN , {H}_{2} S , HCl} {\dot{N}}_{s}} - \frac{{V}_{{CH}_{4}}}{{V}_{others}} \leq {s}_{{ratio}_{3}}$$

$$\frac{{\dot{N}}_{{CH}_{4}}}{{\sum }_{s = {N}_{2} , {NH}_{3} , HCN , {H}_{2} S , HCl} {\dot{N}}_{s}} - \frac{{V}_{{CH}_{4}}}{{V}_{others}} \geq - {s}_{{ratio}_{3}}$$

$${\dot{N}}_{s} \geq 0$$

$$∀ s ∈ {S}_{Pyr , Coal}$$

$${s}_{ratio} \geq 0$$

$$∀ ratio ∈ Ratio$$

Solving this model results in the Nₛ values listed in Table 12, and the final pyrolysis reaction for the given coal composition is given as follows:

C_{6.687}H_{5.387}O_{0.566}N_{0.113}S_{0.113}→5.151C₍ₛ₎+0.431CO+0.067CO₂+0.505H₂+1.004CH₄+0.023N₂+0.0034NH₃+0.034HCN+0.113H₂S (59)

TABLE 12
Results of Coal Pyrolysis Calculation

| | |
|---|---|
| C₍ₛ₎^{FC} = 4.353 | N_{C}_{₍ₛ₎} = 0.797 |
| N_{CO} = 0.431 | N_{CO}_{₂} = 0.067 |
| N_{H}_{₂} = 0.505 | N_{CH}_{₄} = 1.004 |
| N_{N}_{₂} = 0.023 | N_{NH}_{₃} = 0.034 |
| N_{HCN} = 0.034 | N_{H}_{₂}_{S} = 0.113 |

^{a}C₍ₛ₎^{FC} = FCₐ/AWₐ.

### Example 1.15

### Oxidation

After pyrolysis has occurred, the residual gases and char will be exposed to oxygen to generate the necessary heat for gasification. The following oxidation assumptions are made:

O1. H₂ will be fully oxidized to H₂O, because of its high burning velocity, relative to the other hydrocarbons.
O2. The residual O₂ will rapidly combust the char via partial and complete oxidation.
O3. All other gaseous hydrocarbons will have negligible oxidation reactions.40,51 The oxidation reaction list, based on the previous assumptions, consists of the complete combustion of char (eq 60), the partial combustion of char (eq 61), and the combustion of hydrogen (eq 62).

C₍ₛ₎+O₂→CO₂ (60)

C₍ₛ₎+0.5O₂→CO (61)

H₂+0.5O₂→H₂O (62)

### Example 1.16

### Reduction

The heat generated from the oxidation section of the gasifier will facilitate the endothermic reduction reactions that occur during the steam reforming of the char and light hydrocarbons. The assumptions for the reduction section are as follows:

R1. The residual char from the oxidation zone will undergo heterogeneous reactions with the vapor phase.
R2. The vapor phase will be in thermodynamic equilibrium, with respect to the water-gas-shift reaction.
R3. All hydrocarbons will undergo a steam reforming reaction.
The reaction list for the reduction zone is then defined as

C₍ₛ₎+CO₂→2CO (63)

C₍ₛ₎+H₂O→CO+H₂ (64)

C₍ₛ₎+2H₂→CH₄ (65)

CO+H₂O→CO₂+H₂ (66)

CH₄+H₂O→CO+3H₂ (67)

C₂H₂+2H₂O→2CO+3H₂ (68)

C₂H₄+2H₂O→2CO+4H₂ (69)

C₂H₆+2H₂O→2CO+5H₂ (70)

### Example 1.17

### Gasifier Model

In this example, the indices, sets, parameters, variables, assumptions, and mathematical constraints that describe the mathematical model of the gasifiers are described.

Indices. The following indices are used throughout the mathematical model:

a: Atom index
s: Species index
x: Oxidizing input index
f: Feedstock input index
r: Reaction index

Sets. The set of all atoms, A, is given as follows:

a∈A={Ar,C,H,O,N,S,Cl}

Note that A does not include metallic elements that will comprise the ash component of biomass or coal. It is assumed that the ash portion of the feedstock will remain inert and, thus, will have no residual effect on the gasification chemistry. The set of all species, S, present in the gasifier is given as

s∈S={Ar,C₍ₛ₎,CH₄,CO,COS,CO₂,C₂H₂,C₂H₄,C₂H₆,C₅H₈O₄,C₆H₁₀O₅,C₁₅H₁₄O₄,C₂₀H₂₂O₁₀,C₂₂H₂₈O₉,HCN,HCl,H₂,H₂O,H₂S,NH₃,NO,N₂,N₂O,O₂}

where each species is present in the vapor state except for coal, biomass monomers, and char. Representative compounds within the set S are given by the following:
C₍ₛ₎: Char
C₅H₈O₄: Hemicellulose
C₆H₁₀O₅: Cellulose
C₁₅H₁₄O₄: Lig-C
C₂₀H₂₂O₁₀: Lig-O
C₂₂H₂₈O₉: Lig-H
C_{6.69}H_{5.39}O_{0.57}N_{0.11}S_{0.11}: Coal

The hemicellulose, cellulose, and lignin monomers comprise a set of species, S_{Biomass}, that are present in a dry, ash-free (daf) biomass:

S_{Biomass}∈S={C₅H₈O₄,C₆H₁₀O₅,C₁₅H₁₄O₄,C₂₀H₂₂O₁₀,C₂₂H₂₈O₉}

The set of species that will be present in the vapor phase, S_{V}, is given by

S_{V}={Ar,CH₄,CO,COS,CO₂,C₂H₂,C₂H₄,C₂H₆,HCN,HCl,H₂,H₂O,H₂S,NH₃,NO,N₂,N₂O,O₂}

The set of all hydrocarbon species, S_{HC}, is given by

S_{HC}={CH₄,C₂H₂,C₂H₄,C₂H₆}

The set of all compounds that contain a particular atom a is defined as Sₐ and is given by

Sₐ={s∈S:s contains atom a}

To represent all possible oxidizing feeds, we formulate the set Ox by

x∈Ox={Oxygen,Steam,Air,Enriched Air}

where each feed x is described by a set of species Sₓ.

The set of all feedstock types, F, is given as follows:

f∈F={Coal,Biomass,Additional,Fuel}

The set of all reactions, R, within the system is defined as the union of all reactions occurring within the pyrolysis (eqs 11-15, eq 59), oxidation (eqs 60-62), and reduction (eqs 63-70) zones. The set R is subdivided into subsets for the pyrolysis zone (R_{Pyr}), oxidation zone (R_{Ox}), and reduction zone (R_{Red}), respectively.
R_{Pyr}={R(11)-R(15), R(59)}
R_{Ox}={R(60)-R(62)}
R_{Red}={R(63)-R(70)}

Parameters. The composition of the biomass and coal feedstocks correspond to the following set of parameters that represent the dry, ash-free (daf) feedstock:

W_{a,f}: weight fraction of atom a in daf feedstock f
W_{S,Biomass}: weight fraction of species s in daf biomass
E_{a,s}: number of atom a in species s
Note again that the parameters W_{s,Biomass} represent the individual biomass monomers and are generally not reported for a given biomass sample. We utilize the ultimate and proximate analyses of the biomass sample to determine the W_{s,Biomass} value that most
closely approximates this information in Example 1.12.
The following are known inputs to the gasifier:

T: operating temperature of gasifier

M_{f}: input mass flow rate of feedstock f

Fₛ^{LkHp}: molar flow rate of species s in lock hopper carrier gas
F_{s,x}^{Ox}: molar flow rate of species s in oxidizer x
n_{s,r}: molar coefficient of species s in reaction r

Additional parameters are defined by the temperature of the gasifier bed. For each species s, we define the thermodynamic properties as follows:

hs° (T): standard enthalpy of species s at temperature T
gs° (T): standard Gibbs free energy of species s at temperature T
where the functional relationships for hs° (T) and gs° (T) are obtained using NASA polynomial data:

$$\begin{matrix}\frac{{h}_{s}^{°}}{RT} = - {A}_{1} {T}^{- 2} + \frac{{A}_{2} ln ( T )}{T} + {A}_{3} + \frac{{A}_{4} T}{2} + \frac{{A}_{5} {T}^{2}}{3} + \frac{{A}_{6} {T}^{3}}{4} + \frac{{A}_{7} {T}^{4}}{5} + \frac{{A}_{8}}{T} & ( 71 ) \\ \frac{{g}_{s}^{°}}{RT} = - \frac{{A}_{1} {T}^{- 2}}{2} + \frac{2 {A}_{2} ( 1 - ln T )}{T} + {A}_{3} ( 1 - ln T ) - \frac{{A}_{4} T}{2} - \frac{{A}_{5} {T}^{2}}{6} - \frac{{A}_{6} {T}^{3}}{12} - \frac{{A}_{7} {T}^{4}}{20} + \frac{{A}_{8}}{T} - {A}_{9} & ( 72 )\end{matrix}$$

Variables. The variables that are chosen to model the stoichiometric analysis of the gasifier reactions, as well as the composition of the gasifier effluent, are given by the following:

ξᵣ: Molar extent of reaction r

yₛ: Vapor mole fraction of species s

\dot{N}ₐ: Molar flow rate of atom a

\dot{N}ₛ: Molar flow rate of species s

\dot{N}_{T}: Total vapor species molar flow rate

Constraints. The molar atomic flows Nₐ are first defined by summing the molar flow rate contributions from the lockhopper gas (Fₛ^{LkHp}) and the oxidizing gas (F_{s,x}^{Ox}) and the mass flow rate of the feedstocks (M_{f}) using eq 73:

$${\sum }_{s ∈ {S}_{LkHp}} {E}_{a , s} {F}_{s}^{LkHp} + {\sum }_{s ∈ {S}_{Ox}} {\sum }_{x ∈ Ox} {E}_{a , s} {F}_{s , x}^{Ox} + {\sum }_{f ∈ F} {w}_{a , f} {\dot{M}}_{f} {AW}_{a}^{- 1} = {\dot{N}}_{a} \\ ∀ a ∈ A \qquad ( 73 )$$

Note that the molar flow rates for the lockhopper gas and the oxidizing gas are converted to molar atomic flow rates using the parameter E_{a,s}. Both the input steam and oxygen flow rates are included as distinct oxidizing feeds (x∈Ox). The flow rate of input feedstock (i.e., coal, biomass) is generally given as a mass flow rate, so the molar atomic flow rates can be determined using the atomic weight fraction provided by the ultimate analysis, W_{a,f}, and the molar atomic weight (AWₐ).

Through conservation of mass, the molar atomic flow rates can be directly linked to the output species flow rates by eq 74:

$${\sum }_{s ∈ {S}_{a}} {E}_{a , s} {\dot{N}}_{s} = {\dot{N}}_{a} \\ ∀ a ∈ A \qquad ( 74 )$$

The total molar flow rate of all vapor phase species is calculated by

$${\sum }_{s ∈ {S}_{V}} {\dot{N}}_{s} = {\dot{N}}_{T} \qquad ( 75 )$$

The molar composition of the vapor phase species may be obtained using eq 76:

\dot{N}ₛ=yₛ\dot{N}_{T}∀s∈S_{V} (76)

The extents of reaction must be constrained based on the initial molar flow rate of all species and their output molar flow rates, using eq 77:

$${\sum }_{f ∈ F} {w}_{s , f} {\dot{M}}_{f} {MW}_{s}^{- 1} + {F}_{s}^{LkHp} + {\sum }_{x ∈ Ox} {F}_{s , x}^{Ox} - {\dot{N}}_{s} = {\sum }_{r ∈ R} {n}_{s , r} {ξ}_{r} \\ ∀ s ∈ S \qquad ( 77 )$$

where n_{s,r} represents the coefficient of species s in reaction r and is defined to be positive for raw materials and negative for products.

It is initially assumed that the water-gas-shift reaction is at equilibrium, as given by eq 78:

$$\frac{{y}_{{CO}_{2}} {y}_{{H}_{2}}}{{y}_{{H}_{2} O} {y}_{CO}} = exp ( \frac{{g}_{{CO}_{2}}^{°} + {g}_{{H}_{2}}^{°} - {g}_{{H}_{2} O}^{°} - {g}_{CO}^{°}}{RT} ) \qquad ( 78 )$$

Since the temperature of the gasifier is known, each of the values for gₛ° may be explicitly determined from the NASA polynomials listed in eq 72. Therefore, the right-hand side of eq 78 will be equal to a constant.

It is assumed that all pyrolysis reactions go to completion, as represented by eq 79:

\dot{N}ₛ=0∀s∈S_{f} (79)

Thus, upon entering the gasifier, the coal, hemicellulose, cellulose, and lignin compounds will immediately dissociate into the appropriate volatile, char, and tar compounds. To estimate the presence of hydrocarbons in the effluent, an assumption must be made on the steam reforming extent of reaction for each hydrocarbon (eqs 67-70). That is, it is assumed that the fractional conversion of each hydrocarbon formed during pyrolysis is a known parameter, f_{cs}^{HC}. Although it has been previously documented that the fractional conversion of the methane reforming reaction is approximately one-third or less for biomass gasification, it is uncertain what the appropriate value of this parameter should be. An optimization model can be formulated to estimate the value of the parameter that most closely matches the model output to experimental output. The parameter estimation model that finds the appropriate value of f_{cs}^{HC} and all subsequent parameters will be described below. In the model, f_{cs}^{HC} is constrained to be less than or equal to ⅓ for all hydrocarbon components. The hydrocarbon conversions are represented in eq 80:

$${ξ}_{{r}_{s}^{SF}} = {fc}_{s}^{HC} \frac{- {\sum }_{r ∈ {R}_{Pyr}} {ξ}_{r} {n}_{s , r}}{{n}_{s , {r}_{s}^{SF}}} \\ ∀ s ∈ {S}_{HC} \qquad ( 80 )$$

where rₛ^{SF} is the steam reforming reaction associated with species s.

The next set of constraints will dictate the extent of reaction within the oxidation zone. It is initially assumed that all hydrogen present from the pyrolysis reactions and from additional fuel inputs will be immediately oxidized, because of the high burning velocity of this species. That is, all hydrogen formed during pyrolysis (Σ_{r∈R}_{_{Pyr}} ξᵣn_{H}_{₂}_{,r}) will be immediately oxidized to form water (ξ_{R(62)}n_{H}_{₂}_{,R(62)}).

This assumption is represented by eq 81:

$${\sum }_{{r}^{′} ∈ {R}_{Pyr}} {ξ}_{{r}^{′}} {n}_{s , {r}^{′}} + {ξ}_{r} {n}_{s , r} = 0 \\ s = {H}_{2} , r = R ( 62 ) \qquad ( 81 )$$

The remaining oxygen will be consumed by the residual char, because of the high surface area available for O₂ adsorption. The combustion of char will occur via complete (eq 60) and partial (eq 61) oxidation in a ratio that is inversely equal to the exothermicity (Δh) of each reaction:40

$$\frac{{ξ}_{R ( 61 )}}{{ξ}_{R ( 60 )}} = \frac{Δ {h}_{R ( 60 )}}{Δ {h}_{R ( 61 )}} \qquad ( 82 )$$

The exothermicity of each reaction is defined by eqs 83 and 84:

$$\begin{matrix}\frac{Δ {h}_{R ( 61 )}^{o}}{RT} = 2 \frac{{h}_{CO}^{o}}{RT} - \frac{{h}_{{O}_{2}}^{o}}{RT} - 2 \frac{{h}_{C}^{o}}{RT} & ( 83 ) \\ \frac{Δ {h}_{R ( 60 )}^{o}}{RT} = \frac{{h}_{{CO}_{2}}^{o}}{RT} - \frac{{h}_{{O}_{2}}^{o}}{RT} - \frac{{h}_{C}^{o}}{RT} & ( 84 )\end{matrix}$$

where (hₛ°)/(RT) is obtained using NASA polynomial data. Since the operating temperature of the gasifier is known, the value for the exothermicity of each reaction is a constant and the constraint given in eq 82 is linear.

The presence of char and tar in the gasifier exit stream is dependent on the system temperature, as well as the flow rate of oxidizing species input to the gasifier. The model will assume that the tar output by the gasifier is negligible and that the char output is a function of temperature as given by eq 85:

\dot{N}_{C}_{₍ₛ₎}=(a_{Ch}¹+a_{Ch}²T)w_{C}_{₍ₛ₎}_{,f}\dot{M}_{f}AW_{C}_{₍ₛ₎}⁻¹ (85)

where a_{Ch}¹ and a_{Ch}² are coefficients representing the temperature dependence of char output. These coefficients will be varied in the parameter estimation model to determine their optimal values. Although tar is commonly found in biomass gasifiers, because of the low operating temperature, it is removed with a tar cracker before entering the FT unit and, therefore, is not considered in the model.

The next group of constraints focuses on the char reduction reactions (eqs 63-65). It is assumed that the extent of conversion of these reactions will be directly proportional to the initial forward rate of reaction, rateᵣ°. Thus, the three extents are constrained, as in eqs 86 and 87:

$$\begin{matrix}\frac{{ξ}_{R ( 63 )}}{{ξ}_{R ( 64 )}} = \frac{{rate}_{R ( 63 )}^{o}}{{rate}_{R ( 64 )}^{o}} & ( 86 ) \\ \frac{{ξ}_{R ( 64 )}}{{ξ}_{R ( 65 )}} = \frac{{rate}_{R ( 64 )}^{o}}{{rate}_{R ( 65 )}^{o}} & ( 87 )\end{matrix}$$

The rate coefficients are defined using eq 88:

$${k}_{r} = {A}_{r} exp ( \frac{- {E}_{r}}{RT} ) \qquad ( 88 )$$

where Aᵣ is equal to 36.2 s⁻¹ for r=R(63), 1.52×104 s⁻¹ for r=R(64), and 4.19×10−3 s⁻¹ for r=R(65), and Eᵣ is equal to 77.39 kJ/(mol K) for r) R(63), 121.62 kJ/(mol K) for r=
R(64), and 19.21 kJ/(mol K) for r=R(65). Assuming that each char reduction reaction can approximate the elementary rate mechanism, eqs 89 and 90 can relate the reaction rate ratios to the concentrations of the compounds after the oxidation stage.

$$\begin{matrix}\frac{{rate}_{R ( 63 )}^{o}}{{rate}_{R ( 64 )}^{o}} = \frac{{k}_{R ( 63 )} {\dot{N}}_{{CO}_{2}}^{o}}{{k}_{R ( 64 )} {\dot{N}}_{{H}_{2} O}^{o}} & ( 89 ) \\ \frac{{rate}_{R ( 64 )}^{o}}{{rate}_{R ( 65 )}^{o}} = \frac{{k}_{R ( 64 )} {\dot{N}}_{{H}_{2} O}^{o}}{{k}_{R ( 65 )} {\dot{N}}_{{H}_{2}}^{{o}_{2}}} & ( 90 ) \\ {\dot{N}}_{s}^{o} = {\sum }_{r ∈ {R}_{Pyr}} {ξ}_{r} {n}_{s , r} + {\sum }_{r ∈ {R}_{Ox}} {ξ}_{r}^{E} {n}_{s , r } & ( 91 )\end{matrix}$$

Assuming that the oxidative reactions can consume all of the oxygen, the extents of these reactions (ξᵣ^{E}) can be estimated to calculate the molar flow rate of species s(\dot{N}ₛ^{o}) after both pyrolysis and oxidation. Using these estimated values and the given temperature, the initial forward rates of reaction for the char reduction reactions are known, making the right-hand side of eqs 86 and 87 equal to a constant.

The relative proportions of fuel nitrogen present in the vapor phase are constrained. Nitrogen is mostly present as N₂ and NH₃. Hence, it is assumed that the total molar fraction of nitrogen present as these two species is mf_{N}, which is a parameter to be optimized. This parameter is constrained so that mf_{N}≧0.9.57,58

mf_{N}w_{N,f}\dot{M}_{f}AW_{N}⁻¹=\dot{N}_{N}_{₂}MW_{N}_{₂}+\dot{N}_{NH}_{₃}MW_{NH}_{₃} (92)

It is assumed that the relative proportion of N₂ and NH₃ in the effluent is not dependent on the equilibrium, but rather is a linear function of the system temperature.

\dot{N}_{N}_{₂}=a_{N}_{₂}¹+a_{N}_{₂}²(T+a_{N}_{₂}³)(\dot{N}_{N}_{₂}+\dot{N}_{NH}_{₃}) (93)

where a_{N2}¹, a_{N2}², and a_{N2}³ are the parameter values to be optimized. It has also been predicted that the relative ratio of HCN to NH₃ may be a function of the H/N content of the fuel, while the relative ratio of N₂O to NO may be a function of the O/N content
of the fuel. These two assumptions are detailed in eqs 94 and 95:

$$\begin{matrix}{\dot{N}}_{{NH}_{3}} = ( {a}_{{NH}_{3}}^{1} \frac{{w}_{H , f}}{{w}_{N , f}} + {a}_{{NH}_{3}}^{2} ) {\dot{N}}_{HCN} & ( 94 ) \\ {\dot{N}}_{NO} = ( {a}_{NO}^{1} \frac{{w}_{O , f}}{{w}_{N , f}} + {a}_{NO}^{2} ) {\dot{N}}_{{N}_{2} O} & ( 95 )\end{matrix}$$

where a_{N2}¹=2.359×10⁻⁴, a_{N2}¹=2.181×10⁻³, a_{NO}¹=2.634×10⁻⁴, and a_{NO}²=0.1111. These values are determined by a linear regression method from experimental data presented in Table 4 of Stubenberger et al, 2008, which is incorporated herein by reference as if fully set forth.

The final set of constraints involves the sulfur species present in the gasifier effluent. Little has been reported on the characteristics of the sulfur present in the gasifier effluent. The decomposition of sulfur is distributed between H₂S and

COS, as represented in eq 96:

fc_{S}w_{S,f}\dot{M}_{f}AW_{S}⁻¹=\dot{N}_{H}_{₂}_{S} (96)

where fcs is the fractional conversion of fuel sulfur to H₂S and the optimal value of the will be determined using parameter estimation.

Objective Function. The output composition of the gasifier unit can be calculated by minimizing the output oxygen from the gasifier (eq 97):

$${min}_{{ξ}_{r} , {y}_{s} , {\dot{N}}_{a} , {\dot{N}}_{s}} {\dot{N}}_{{O}_{2}} \qquad ( 97 )$$

After the aforementioned marked parameters have been assigned specific values, the constraints define a system of equations that has only one degree of freedom. To develop a square system of equations, the outlet oxygen flow rate from the gasifier would be set to zero, which is anticipated during actual operation. A feasibility model is then established by minimizing the outlet flow of oxygen. Note that the optimization model is solved separately for the coal and biomass gasifiers.

Parameter Estimation. The constraints listed above (eqs 73-96) detail the gasifier model, which has several key unknown parameters. Before the gasifier model can be used in conjunction with the CBGTL process, a nonlinear parameter optimization must be performed to determine the optimal values. Several case studies have been used to compare the experimental output to the model predictions. A Euclidean distance metric is used to compute the validity of the model output. The experimental values reported in the literature are often missing several of the lower abundance gases, including hydrocarbons, sulfur species, nitrogen species, and chlorine species. All experimental mole fractions are calculated and normalized so that they sum to 1. To ensure that the comparison between experimental and theoretical values is as accurate as possible, all of the vapor phase mole fractions in the mathematical model are normalized appropriately. For instance, assuming that the species reported in a given experiment e are defined by the set Se, then the normalized vapor phase mole fractions are given by eq 98:

$${y}_{s , e} {\sum }_{s ∈ {S}_{e}} {y}_{s} = {y}_{s} \qquad ( 98 )$$

The normal vapor-phase mole fraction reported by the gasifier model, Yₛ, has now been converted to a normalized fraction, Y_{s,e}, so that a direct match to a particular experimental value can be made. The distance metric used is presented in eq 99:

$$\begin{matrix}{ED}_{e} = \sqrt{{\sum }_{s ∈ {S}_{e}} {( {y}_{s , e} - {y}_{s , e}^{exp} )}^{2}} & ( 99 ) \\ Dist = \frac{{\sum }_{e ∈ E} {ED}_{e}}{\vert E \vert} & ( 100 )\end{matrix}$$

where Y_{s,e}^{exp} is the experimental value and E is the set of all experimental case studies. The objective of the nonlinear parameter estimation model is to minimize the average overall distance (eq 100) when considering all case studies. It is important to note that,
for the nonlinear parameter estimation model, all of the variables are defined over the index e, as well as the original indices. Each experimental case study requires a distinct output from the gasification model, so all of the variables must be able to change when considering a different case study. The only variables that remain constant over all of the experiments are the parameters that are optimized (Table 13, below).

TABLE 13
Parameters Being Optimized in the Gasifier Model

| parameter | biomass | coal |
|---|---|---|
| mf_{N} | 0.9801 | 1 |
| fc_{S} | 0.5030 | 1 |
| fc_{CH}_{₄}^{HC} | ⅓ | ⅓ |
| fc_{C}_{₂₂}^{HC} | ⅓ | |
| fc_{C}_{₂}_{H}_{₄}^{HC} | ⅓ | |
| fc_{C}_{₂}_{H}_{₆}^{HC} | ⅓ | |
| a_{Ch}¹ | 5.002 × 10⁻³ | 0.271 |
| a_{Ch}² | 1.132 × 10⁻⁶ | 0 |
| a_{N}_{₂}¹ | 0.4001 | −0.9310 |
| a_{N}_{₂}² | 1.25 × 10⁻³ | 0.6976 |
| a_{N}_{₂}³ | −1075 | −1000 |
| a_{NH}_{₃}¹ | 2.359 × 10⁻⁴ | 2.359 × 10⁻⁴ |
| a_{NH}_{₃}² | 2.818 × 10⁻³ | 2.818 × 10⁻³ |
| a_{NO}¹ | 2.634 × 10⁻⁴ | 2.634 × 10⁻⁴ |
| a_{NO}² | 0.1111 | 0.1111 |

The comparison of theoretical and experimental output for the biomass and coal nonlinear parameter estimation models can be found in Tables 14 and 15, respectively. This comparison reveals that the model performs well in representing the gasification process. A feature of the model is its generality in evaluating syngas compositions for a variety of feedstock and gasifier types. The values of the parameters which provide the predicted results are given in Table 13. These values are used to define the biomass and coal gasifiers used in the CBGTL process.

The full mathematical models are included below, with the corresponding parameters substituted into the equations.

Biomass Gasifier Model.

$${min}_{{ξ}_{r} , {y}_{s} , {\dot{N}}_{a} , {\dot{N}}_{s}} {\dot{N}}_{{O}_{2}}$$

$$subject to$$

$${\sum }_{s ∈ {S}_{LkHp}} {E}_{a , s} {F}_{s}^{LkHp} + {\sum }_{s ∈ {S}_{Ox}} {\sum }_{x ∈ Ox} {E}_{a , s} {F}_{s , x}^{Ox} + {w}_{a , Biomass} {\dot{M}}_{Biomass} {AW}_{a}^{- 1} = {\dot{N}}_{a}$$

$$∀ a ∈ A$$

$${\sum }_{s ∈ {S}_{a}} {E}_{a , s} {\dot{N}}_{s} = {\dot{N}}_{a} ∀ a ∈ A$$

$${\sum }_{s ∈ {S}_{V}} {\dot{N}}_{s} = {\dot{N}}_{T}$$

$${\dot{N}}_{s} = {y}_{s} {\dot{N}}_{T} ∀ s ∈ {S}_{V}$$

$${w}_{s , Biomass} {\dot{M}}_{Biomass} {MW}_{s}^{- 1} + {\sum }_{x ∈ Ox} {F}_{s , x}^{Ox} - {\dot{N}}_{s} = {\sum }_{r ∈ R} {n}_{s , r} {ξ}_{r} ∀ s ∈ S$$

$$\frac{{y}_{{CO}_{2}} {y}_{{H}_{2}}}{{y}_{{H}_{2} O} {y}_{CO}} = exp ( \frac{{g}_{{CO}_{2}}^{o} + {g}_{{H}_{2}}^{o} - {g}_{{H}_{2} O}^{o} - {g}_{CO}^{o}}{RT} )$$

$${\dot{N}}_{s} = 0 ∀ s ∈ {S}_{Biomass}$$

$${ξ}_{{r}_{s}^{SF}} = \frac{1}{3} \frac{- {\sum }_{r ∈ {R}_{Pyr}} {ξ}_{r} {n}_{s , r}}{{n}_{s , {r}_{s}^{SF}}} ∀ s ∈ {S}_{HC}$$

$${\sum }_{{r}^{′} ∈ {R}_{Pyr}} {ξ}_{{r}^{′}} {n}_{s , {r}^{′}} + {ξ}_{r} {n}_{s , r} = 0 s = {H}_{2} , r = R ( 62 )$$

$$\frac{{ξ}_{R ( 61 )}}{{ξ}_{R ( 60 )}} = \frac{Δ {h}_{R ( 60 )}}{Δ {h}_{R ( 61 )}}$$

$${\dot{N}}_{{C}_{( s )}} = ( 5.002 {E}^{- 3} + 1.132 {E}^{- 6} T ) {w}_{{c}_{( s )} Biomass} {\dot{M}}_{Biomass} {AW}_{{C}_{( s )}}^{- 1}$$

$$\frac{{ξ}_{R ( 63 )}}{{ξ}_{R ( 64 )}} = \frac{{rate}_{R ( 63 )}^{o}}{{rate}_{R ( 64 )}^{o}}$$

$$\frac{{ξ}_{R ( 64 )}}{{ξ}_{R ( 65 )}} = \frac{{rate}_{R ( 64 )}^{o}}{{rate}_{R ( 65 )}^{o}}$$

$$0.9801 ( {w}_{N , Biomass} {\dot{M}}_{Biomass} ) {A W}_{N}^{- 1} = {\dot{N}}_{{N}_{2}} {MW}_{{N}_{2}} + {\dot{N}}_{{NH}_{3}} {MW}_{{NH}_{3}}$$

$${\dot{N}}_{{N}_{2}} = 0.4001 + 1.25 {E}^{- 3} ( T - 1075 ) ( {\dot{N}}_{{N}_{2}} + {\dot{N}}_{{NH}_{3}} )$$

$${\dot{N}}_{{NH}_{3}} = ( 2.359 {E}^{- 4} \frac{{w}_{H , Biomass}}{{w}_{N , Biomass}} - 2.818 {E}^{- 3} ) {\dot{N}}_{HCN}$$

$${\dot{N}}_{NO} = ( 2.634 {E}^{- 4} \frac{{w}_{O , Biomass}}{{w}_{N , Biomass}} + 0.1111 ) {\dot{N}}_{{N}_{2} O}$$

$$0.5030 {w}_{s , Biomass} {\dot{M}}_{Biomass} {AW}_{S}^{- 1} = {\dot{N}}_{{H}_{2} S}$$

Coal Gasifier Model.

$${min}_{{ξ}_{r} , {y}_{s} , {\dot{N}}_{a} , {\dot{N}}_{s}} {\dot{N}}_{{O}_{2}}$$

$$subject to$$

$${\sum }_{s ∈ {S}_{LkHp}} {E}_{a , s} {F}_{s}^{LkHp} + {\sum }_{s ∈ {S}_{Ox}} {\sum }_{x ∈ Ox} {E}_{a , s} {F}_{s , x}^{Ox} + {w}_{a , Coal} {\dot{M}}_{Coal} {AW}_{a}^{- 1} = {\dot{N}}_{a}$$

$$∀ a ∈ A$$

$${\sum }_{s ∈ {S}_{a}} {E}_{a , s} {\dot{N}}_{s} = {\dot{N}}_{a} ∀ a ∈ A$$

$${\sum }_{s ∈ {S}_{V}} {\dot{N}}_{s} = {\dot{N}}_{T}$$

$${\dot{N}}_{s} = {y}_{s} {\dot{N}}_{T} ∀ s ∈ {S}_{V}$$

$${w}_{s , Coal} {\dot{M}}_{Coal} {MW}_{s}^{- 1} + {\sum }_{x ∈ Ox} {F}_{s , x}^{Ox} - {\dot{N}}_{s} = {\sum }_{r ∈ R} {n}_{s , r} {ξ}_{r} ∀ s ∈ S$$

$$\frac{{y}_{{CO}_{2}} {y}_{{H}_{2}}}{{y}_{{H}_{2} O} {y}_{CO}} = exp ( \frac{{g}_{{CO}_{2}}^{o} + {g}_{{H}_{2}}^{o} - {g}_{{H}_{2} O}^{o} - {g}_{CO}^{o}}{RT} )$$

$${\dot{N}}_{s} = 0 ∀ s ∈ {S}_{Coal}$$

$${ξ}_{{r}_{s}^{SF}} = \frac{1}{3} \frac{- {\sum }_{r ∈ {R}_{Pyr}} {ξ}_{r} {n}_{s , r}}{{n}_{s , {r}_{s}^{SF}}} ∀ s ∈ {S}_{HC}$$

$${\sum }_{{r}^{′} ∈ {R}_{Pyr}} {ξ}_{{r}^{′}} {n}_{s , {r}^{′}} + {ξ}_{r} {n}_{s , r} = 0 s = {H}_{2} , r = R ( 62 )$$

$$\frac{{ξ}_{R ( 61 )}}{{ξ}_{R ( 60 )}} = \frac{Δ {h}_{R ( 60 )}}{Δ {h}_{R ( 61 )}}$$

$${\dot{N}}_{{C}_{( s )}} = 0.271 {w}_{{C}_{( s )} , Coal} {\dot{M}}_{Coal} {AW}_{{C}_{( s )}}^{- 1}$$

$$\frac{{ξ}_{R ( 63 )}}{{ξ}_{R ( 64 )}} = \frac{{rate}_{R ( 63 )}^{o}}{{rate}_{R ( 64 )}^{o}}$$

$$\frac{{ξ}_{R ( 64 )}}{{ξ}_{R ( 65 )}} = \frac{{rate}_{R ( 64 )}^{o}}{{rate}_{R ( 65 )}^{o}}$$

$$1.0 ( {w}_{N , Coal} {\dot{M}}_{Coal} ) {A W}_{N}^{- 1} = {\dot{N}}_{{N}_{2}} {MW}_{{N}_{2}} + {\dot{N}}_{{NH}_{3}} {MW}_{{NH}_{3}}$$

$${\dot{N}}_{{N}_{2}} = - 0.9310 + 0.6976 ( T - 1000 ) ( {\dot{N}}_{{N}_{2}} + {\dot{N}}_{{NH}_{3}} )$$

$${\dot{N}}_{{NH}_{3}} = ( 2.359 {E}^{- 4} \frac{{w}_{H , Coal}}{{w}_{N , Coal}} - 2.818 {E}^{- 3} ) {\dot{N}}_{HCN}$$

$${\dot{N}}_{NO} = ( 2.634 {E}^{- 4} \frac{{w}_{O , Coal}}{{w}_{N , Coal}} + 0.1111 ) {\dot{N}}_{{N}_{2} O}$$

$$1.0 {w}_{s , Coal} {\dot{M}}_{Coal} {AW}_{S}^{- 1} = {\dot{N}}_{{H}_{2} S}$$

TABLE 14
Vapor Effluent Comparisons with Reported Biomass Gasification Tests^{a}

| | Model wt % (Reported wt %) | | | | | | | | | | | | |
|---|---|---|---|---|---|---|---|---|---|---|---|---|---|
| No. | CO | CO₂ | H₂ | H₂O | CH₄ | C₂H₄ | C₂H₆ | N₂ | NH₃ | H₂S | HCl | Ar | ED^{b} |

Data Taken from van der Drift et al.⁶¹

| | | | | | | | | | | | | | |
|---|---|---|---|---|---|---|---|---|---|---|---|---|---|
| 1 | 7.348 (8.06) | 17.1 (14.66) | 6.779 (6.17) | 16.06 (14.26) | 4.155 (2.83) | 1.06 (0.94) | 0.08997 (0.086) | 46.64 (51.85) | 0.1952 (0.15) | 0.005743 (0.0043) | | 0.5571 (0.5) | 3.44191 |
| 2 | 16.95 (10.23) | 12.33 (14.19) | 11.24 (6.35) | 8.89 (9.9) | 1.687 (2.93) | 0.8609 (0.77) | 0.108 (0.027) | 47.26 (54.44) | 0.09435 (0.19) | | 0.01696 (0.018) | 0.5653 (0.55) | 8.66715 |
| 3 | 13.03 (10.7) | 14.31 (14.25) | 7.513 (6.22) | 9.378 (8.14) | 3.307 (2.91) | 0.6803 (0.91) | 0.08259 (0.037) | 51.03 (55.67) | 0.05009 (0.12) | | 0.01484 (0.00018) | 0.6109 (0.57) | 2.97594 |
| 4 | 10.88 (8.45) | 15.09 (13.7) | 7.972 (5.63) | 11.85 (12.34) | 2.992 (2.43) | 1.144 (0.76) | 0.08297 (0.026) | 49.08 (55.16) | 0.2916 (0.49) | 0.01022 (0.0044) | 0.0203 (0.00018) | 0.585 (0.55) | 3.75095 |
| 5 | 9.411 (8.86) | 16.6 (13.99) | 6.429 (6.53) | 10.63 (10.37) | 3.629 (2.31) | 1.424 (1.03) | 0.07679 (0.045) | 50.57 (54.68 ) | 0.6275 (1.12) | | | 0.6009 (0.5) | 3.05651 |
| 6 | 10.26 (9.75) | 16.15 (13.91) | 6.805 (7.74) | 10.47 (8.36) | 3.665 (2.56) | 1.438 (0.95) | 0.07756 (0.037) | 49.72 (54.5) | 0.604 (1.15) | | 0.2084 (0.00018) | 0.5905 (0.55) | 3.52249 |
| 7 | 7.416 (6.91) | 16.25 (13.35) | 6.912 (4.48) | 14.3 (16.79) | 2.182 (1.43) | 1.221 (0.5) | 0.1049 (0.017) | 50.74 (55.4) | 0.2464 (0.26) | 0.02795 (0.0191) | 0.005462 (0.00017) | 0.606 (0.55) | 4.67731 |
| 8 | 14.62 (9.27) | 13.31 (12.23) | 9.635 (5.13) | 9.784 (12.3) | 3.207 (2.53) | 0.7527 (0.83) | 0.05261 (0.026) | 48 (56.43) | 0.0521 (0.04) | 0.01251 (0.0123) | 0.00362 (0.00026) | 0.5746 (0.53) | 7.54197 |
| 9 | 10.73 (7.17) | 15.03 (14.37) | 8.257 (8.09) | 11.58 (10.32) | 3.352 (2.1) | 1.199 (1.01) | 0.0802 (0.045) | 48.41 (55.11) | 0.7561 (0.73) | 0.02767 (0.0018) | 0.003635 (9e-05) | 0.5731 (0.52) | 4.04145 |

Data Taken from Hsi et al.⁶⁶

| | | | | | | |
|---|---|---|---|---|---|---|
| 10 | 13.93 (18.93) | 16.38 (10.6) | 12.68 (10.99) | 1.397 (1.69) | 55.61 (56.34) | 7.83265 |
| 11 | 15.47 (20.74) | 15.77 (9.04) | 14.24 (15.15) | 1.44 (1.52) | 53.08 (52.25) | 8.59653 |
| 12 | 16.05 (22.19) | 15.54 (6.66) | 14.83 (16.45) | 1.456 (1.31) | 52.12 (51.51) | 10.9179 |
| 13 | 17.65 (20.49) | 14.91 (8.9) | 16.49 (19.85) | 1.501 (1.41) | 49.44 (48.25) | 7.44873 |
| 14 | 15.76 (19.68) | 15.66 (10.18) | 14.53 (12.13) | 1.448 (1.52) | 52.6 (55.02) | 7.15276 |
| 15 | 16.34 (20.52) | 15.42 (9.18) | 15.14 (17.8) | 1.465 (1.58) | 51.63 (49.56) | 7.96861 |
| 16 | 18.17 (20.25) | 14.71 (9.29) | 17.03 (17.2) | 1.516 (1.68) | 48.57 (49.94) | 5.81021 |
| 17 | 22.99 (23.68) | 11.3 (7.14) | 15.4 (17.47) | 1.28 (1.91) | 49.03 (48.24) | 4.73957 |
| 18 | 11.12 (18.39) | 18.54 (10.97) | 13.81 (14.59) | 1.701 (1.47) | 54.83 (53.62) | 10.5271 |
| 19 | 7.71 (19.64) | 19.63 (9.51) | 6.882 (11.16) | 3.178 (1.64) | 62.6 (55.79) | 16.2913 |
| 20 | 10.77 (18.67) | 19.4 (9.86) | 12.5 (13.23) | 4.105 (1.48) | 53.23 (54.46) | 12.6825 |
| 21 | 13.17 (16.79) | 18.12 (10.58) | 14.07 (16.43) | 3.975 (1.41) | 50.66 (52.42) | 9.06117 |
| 22 | 13.8 (14.83) | 18.66 (11.05) | 17.05 (19.05) | 4.545 (1.22) | 45.94 (51.07) | 8.60399 |
| 23 | 15.87 (17.94) | 16.94 (11.14) | 16.43 (18.3) | 4.03 (1.23) | 46.73 (49.46) | 7.01868 |
| 24 | 14.39 (16.68) | 17.65 (10.75) | 15.33 (17.99) | 4.039 (1.36) | 48.59 (50.73) | 8.19187 |

Data Taken from Faaij et al.⁶⁷

| | | | | | | | | | | | | | |
|---|---|---|---|---|---|---|---|---|---|---|---|---|---|
| 25 | 19.65 (17.22) | 10.53 (12.22) | 17.04 (13.25) | 11.67 (13.55) | 1.757 (2.82) | 0.534 (0.94) | 0.06089 (0.02) | 38.2 (39.2) | 0.07944 (0.27) | | 0.02389 (0.03) | 0.4565 (0.47) | 5.29079 |
| 26 | 15.51 (14.94) | 11.66 (12.09) | 13.84 (12.42) | 13.3 (14.49) | 2.443 (2.61) | 0.7452 (0.87) | 0.0378 (0.02) | 41.56 (41.64) | 0.2988 (0.33) | 0.01757 (0.03) | 0.08853 (0.07) | 0.4934 (0.5) | 1.9969 |
| 27 | 14.21 (13.98) | 11.24 (11.8) | 12.8 (11.27) | 12.94 (13.71) | 1.954 (2.81) | 0.9985 (0.77) | 0.02365 (0.02) | 44.94 (44.59) | 0.3062 (1) | 0.05553 (0.03) | | 0.5338 (0.54) | 2.13713 |
| 28 | 19.57 (18.31) | 10.47 (11.67) | 17.77 (15.07) | 12.15 (13.85) | 1.8 (2.93) | 0.5321 (0.98) | 0.07852 (0.02) | 37.13 (36.64) | 0.02516 (0.07) | 0.004019 (0.01) | 0.01937 (0.02) | 0.4445 (0.44) | 3.83283 |
| 29 | 16.38 (15.18) | 9.45 (12.22) | 14.79 (12.37) | 10.91 (14.34) | 1.451 (2.63) | 1.111 (0.88) | 0.01152 (0.02) | 44.31 (41.04) | 0.7952 (0.78) | 0.2441 (0.04) | 0.03425 (0.01) | 0.5186 (0.49) | 5.31234 |

Data Taken from Jayah et al.⁶⁸

| | | | | | | |
|---|---|---|---|---|---|---|
| 30 | 24.49 (19.6) | 9.966 (9.9) | 20.08 (17.2) | 2.466 (1.4) | 42.99 (51.9) | 5.7747 |
| 31 | 19.91 (20.2) | 11.8 (9.7) | 15.1 (18.3) | 2.216 (1.1) | 50.97 (50.7) | 3.99744 |
| 32 | 18.56 (19.4) | 12.33 (9.7) | 13.62 (17.2) | 2.131 (1.1) | 53.36 (52.6) | 4.63701 |
| 33 | 22.33 (18.4) | 10.73 (10.6) | 17.33 (17) | 2.311 (1.3) | 47.3 (52.7) | 4.07343 |
| 34 | 20.86 (19.7) | 11.33 (10.8) | 15.77 (13.2) | 2.233 (1.3) | 49.8 (55) | 3.01693 |
| 35 | 19.45 (18.9) | 11.89 (8.5) | 14.25 (12.5) | 2.149 (1.2) | 52.26 (59.1) | 3.9696 |
| 36 | 23.66 (19.1) | 10.06 (11.4) | 18.21 (15.5) | 2.327 (1.1) | 45.73 (52.9) | 5.60703 |
| 37 | 21.97 (22.1) | 10.75 (10.5) | 16.42 (12.7) | 2.237 (1.3) | 48.62 (53.4) | 3.84653 |
| 38 | 19.02 (19.1) | 12 (10.7) | 13.55 (13) | 2.095 (1.2) | 53.33 (56) | 1.6733 |

Data Taken from Navaez et al.⁴³

| | | | | | | |
|---|---|---|---|---|---|---|
| 39 | 14.32 (16.2) | 17.22 (12.7) | 9.275 (9.2) | 5.594 (2.5) | 53.58 (59.6) | 5.79165 |
| 40 | 21.77 (20.5) | 14 (12.9) | 14.1 (11.5) | 6.357 (4.1) | 43.78 (50) | 3.83105 |
| 41 | 4.568 (9.2) | 21.47 (12.8) | 3.007 (5.9) | 4.617 (0.6) | 66.34 (71) | 11.0059 |
| 42 | 23.25 (20.2) | 13.25 (11.8) | 14.65 (14.7) | 6.44 (4.4) | 42.42 (49) | 3.94577 |
| 43 | 20.82 (20) | 14.41 (12.4) | 13.5 (11.7) | 6.262 (3.3) | 45 (53) | 4.08974 |

^{a}Data taken from van der Drift et al.,
⁶¹unless noted otherwise.
^{b}The Euclidean distance (ED) is used as a meteric for comparison of the experimental output to the theoretical output.

TABLE 15
Vapor Effluent Comparison with Reported Coal Gasification Tests

| | Model dry wt % (Reported dry wt %) | | | | | | |
|---|---|---|---|---|---|---|---|
| case study | CO | CO₂ | H₂ | CH₄ | N₂ | Ar | ED |

Data Taken from Li et al.³⁵

| | | | | | | |
|---|---|---|---|---|---|---|
| 1 | 10.67 (10.20) | 14.32 (15.70) | 9.46 (10.00) | 0.07 (1.00) | 65.48 (65.10) | 2.30 |
| 2 | 6.75 (9.10) | 13.13 (15.00) | 5.18 (5.60) | 0.05 (0.50) | 71.85 (69.80) | 3.69 |
| 3 | 10.67 (12.00) | 12.63 (13.10) | 8.73 (8.50) | 0.06 (0.80) | 66.43 (65.60) | 1.81 |
| 4 | 13.86 (13.40) | 12.83 (13.30) | 12.73 (10.40) | 0.07 (1.00) | 60.50 (61.90) | 2.95 |
| 5 | 6.77 (10.10) | 12.30 (14.20) | 5.31 (5.60) | 0.05 (0.50) | 71.60 (69.60) | 4.36 |
| 6 | 10.76 (13.20) | 11.87 (12.30) | 8.94 (8.40) | 0.06 (0.80) | 66.07 (65.30) | 2.76 |
| 7 | 13.48 (13.60) | 12.93 (13.00) | 11.82 (9.90) | 0.07 (1.00) | 61.70 (62.50) | 2.28 |
| 8 | 14.25 (9.70) | 12.74 (15.50) | 13.68 (8.80) | 0.07 (1.00) | 59.26 (65.10) | 9.33 |

Data Taken from Watkinson et al.⁶⁹

| | | | | | |
|---|---|---|---|---|---|
| 9 | 70.55 (70.50) | 2.00 (1.80) | 27.28 (27.30) | 0.01 (0.40) | 0.44 |
| 10 | 63.93 (61.30) | 3.14 (2.50) | 31.87 (28.10) | 0.17 (8.10) | 9.19 |

Data Taken from Xiao et al.⁷⁰

| | | | | | | |
|---|---|---|---|---|---|---|
| 11 | 9.90 (10.50) | 12.23 (15.30) | 12.11 (10.60) | 0.05 (2.3) | 64.38 (60.30) | 5.81 |
| 12 | 11.42 (11.80) | 12.01 (14.30) | 14.11 (12.30) | 0.06 (2.40) | 61.64 (58.20) | 5.10 |
| 13 | 12.65 (12.20) | 11.09 (13.50) | 15.82 (15.20) | 0.06 (2.40) | 59.19 (55.70) | 4.90 |

Data Taken from Huang et al.⁷¹

| | | | | | | |
|---|---|---|---|---|---|---|
| 14 | 12.11 (13.88) | 11.51 (15.90) | 17.83 (14.57) | 0.04 (2.91) | 57.91 (52.70) | 8.11 |
| 15 | 15.80 (13.97) | 11.10 (13.17) | 24.46 (18.04) | 0.05 (2.93) | 48.59 (51.89) | 8.25 |
| 16 | 10.68 (12.54) | 12.88 (14.74) | 20.72 (18.56) | 0.04 (2.72) | 54.52 (51.44) | 5.32 |
| 17 | 14.52 (14.30) | 11.75 (13.89) | 23.08 (18.08) | 0.05 (2.55) | 50.60 (51.17) | 6.02 |
| 18 | 9.59 (8.02) | 14.44 (17.10) | 19.24 (17.57) | 0.04 (3.59) | 56.69 (53.72) | 5.81 |
| 19 | 11.20 (15.14) | 12.47 (15.40) | 20.37 (16.63) | 0.04 (2.63) | 54.76 (50.20) | 8.10 |

Data Taken from Wang et al.⁷²

| | | | | | | |
|---|---|---|---|---|---|---|
| 20 | 8.33 (9.97) | 12.84 (14.40) | 14.89 (9.63) | 0.10 (1.34) | 63.84 (64.62) | 5.91 |
| 21 | 7.85 (10.94) | 12.77 (19.31) | 12.86 (8.53) | 0.10 (0.84) | 66.40 (60.37) | 10.39 |
| 22 | 4.49 (5.80) | 13.12 (14.86) | 7.40 (6.48) | 0.08 (1.29) | 73.51 (71.54) | 3.31 |

Data Taken from Hobbs et al.⁷³

| | | | | | | | |
|---|---|---|---|---|---|---|---|
| 23 | 28.63 (30.80) | 0.39 (4.06) | 18.78 (17.90) | 0.07 (1.38) | 48.85 (44.90) | 0.58 (0.96) | 6.03 |
| 24 | 21.98 (23.00) | 6.57 (10.10) | 26.04 (20.20) | 0.11 (1.70) | 44.76 (43.60) | 0.54 (1.40) | 7.23 |
| 25 | 32.16 (27.60) | 0.04 (5.10) | 21.03 (17.90) | 0.08 (1.60) | 46.14 (46.50) | 0.55 (1.30) | 7.70 |
| 26 | 28.63 (22.90) | 0.06 (7.50) | 18.57 (16.30) | 0.08 (1.70) | 50.01 (49.70) | 0.60 (1.90) | 9.89 |
| 27 | 29.69 (24.70) | 0.05 (5.67) | 18.29 (17.20) | 0.07 (1.60) | 49.14 (49.60) | 0.59 (1.23) | 7.78 |
| 28 | 30.49 (29.50) | 0.15 (4.96) | 18.83 (16.30) | 0.08 (1.79) | 48.91 (46.30) | 0.59 (1.15) | 6.36 |
| 29 | 30.49 (30.30) | 0.56 (4.90) | 20.13 (18.20) | 0.07 (1.63) | 46.97 (44.00) | 0.56 (0.97) | 5.83 |
| 30 | 27.14 (30.00) | 1.43 (4.47) | 17.71 (16.40) | 0.07 (1.50) | 50.82 (46.50) | 0.61 (1.03) | 6.35 |
| 31 | 31.87 (27.00) | 0.05 (6.25) | 23.60 (18.30) | 0.09 (1.93) | 43.86 (45.30) | 0.53 (1.22) | 9.81 |

Data Taken from Ocampo et al.⁷⁴

| | | | | | | |
|---|---|---|---|---|---|---|
| 32 | 9.64 (10.59) | 12.53 (18.38) | 19.13 (8.84) | 0.11 (1.07) | 58.59 (61.10) | 12.18 |
| 33 | 8.66 (10.71) | 11.89 (20.90) | 16.11 (12.86) | 0.09 (0.83) | 62.22 (54.55) | 12.47 |
| 34 | 8.83 (8.84) | 12.88 (20.12) | 17.63 (9.90) | 0.11 (0.73) | 60.55 (59.97) | 10.63 |
| 35 | 8.81 (11.36) | 12.37 (20.27) | 14.60 (10.10) | 0.10 (0.77) | 64.13 (57.50) | 11.56 |

Data Taken from Shadle et al.⁷⁵

| | | | | | | |
|---|---|---|---|---|---|---|
| 36 | 7.83 (6.60) | 10.70 (11.60) | 8.22 (7.90) | 0.10 (1.50) | 73.15 (73.70) | 2.16 |
| 37 | 4.03 (7.80) | 8.48 (11.50) | 6.85 (8.40) | 0.09 (1.70) | 73.70 (71.20) | 5.88 |
| 38 | 7.77 (6.70) | 11.57 (10.20) | 6.04 (6.50) | 0.07 (1.80) | 73.74 (74.00) | 2.51 |
| 39 | 6.85 (7.00) | 12.11 (8.60) | 5.23 (5.20) | 0.07 (1.60) | 74.75 (77.00) | 4.44 |

### Example 1.18

### Fischer-Tropsch Units

The FT reactors take the clean syngas and convert it to a range of hydrocarbon products. Although the products can be assumed to follow the theoretical ASF distribution (eq 7), the observed yields of the lighter hydrocarbons are higher than what the ASF distribution predicts. These deviations are incorporated in eqs 101-106, which comprise the slightly modified ASF distribution used to model the high-temperature and low temperature FT units.

$$\begin{matrix}{W}_{1} = \frac{1}{2} ( 1 - {\sum }_{n = 5}^{∞} {W}_{n} ) & ( 101 ) \\ {W}_{2} = \frac{1}{6} ( 1 - {\sum }_{n = 5}^{∞} {W}_{n} ) & ( 102 ) \\ {W}_{3} = \frac{1}{6} ( 1 - {\sum }_{n = 5}^{∞} {W}_{n} ) & ( 103 ) \\ {W}_{4} = \frac{1}{6} ( 1 - {\sum }_{n = 5}^{∞} {W}_{n} ) & ( 104 ) \\ {W}_{n} = {n ( 1 - α )}^{2} {α}^{n - 1} ∀ 5 \leq n \leq 29 & ( 105 ) \\ {W}_{Wax} = {\sum }_{n = 30}^{∞} {n ( 1 - α )}^{2} {α}^{n - 1} & ( 106 )\end{matrix}$$

where Wn is the weight fraction of Cn compounds and α is the chain growth probability.

Given the weight fractions, we define the carbon present at each hydrocarbon length, crₙ, as follows:

$${cr}_{n} = \frac{{nW}_{n}}{{\sum }_{n = 1}^{29} {nW}_{n} + {n}_{Wax} {W}_{Wax}} \qquad ( 107 )$$

where crₙ represents the fraction of carbon that is present at chain length n for all desired n.

The input-output relationships between incoming and outgoing species in the FT reactors are given in the following equations:

Fₛ^{CS}=Fₛ^{FT}∀s∈S_{FT}^{Inert} (108)

F_{CO}^{FT,LT}+F_{CO}^{FT,HT}=F_{CO}^{CS} (109)

(1−fc_{CO}^{FT})(F_{CO}^{FT,LT}+F_{CO}^{FT,HT})=F_{CO}^{FT} (110)

crₛ^{FT,LT}fc_{CO}^{FT}F_{CO}^{FT,LT}+crₛ^{FT,HT}fc_{CO}^{FT}F_{CO}^{FT,HT}=Fₛ^{FT}∀s∈S_{FT}^{HC} (111)

where S_{FT}^{Inert} is the set of all inert species that do not participate in the FT reactions, S_{FT}^{HC} is the set of all hydrocarbon species in the FT reactor, Fₛ^{CS} the flow rate of component s in the clean syngas stream, Fₛ^{FT} is the total flow rate of component s exiting both FT reactors, Fₛ^{FT,LT} and Fₛ^{FT,HT} are the flow rate of component s entering the low-temperature FT and the high temperature FT, respectively, fc_{CO}^{FT} is the fractional conversion of CO in the FT reactor, which is assumed to be 0.8, and crₛ is calculated for each species s, based on the chain length of the species and the relative proportions of paraffins and olefins. Equation 108 sets the inlet and outlet flow rates for components that do not participate in the FT reactions equal to each other. Equation 109 models the splitting of the syngas stream into the two types of FT reactors. Unconverted CO exits the two reactors, as defined by eq 110, while the exiting composition of the remaining hydrocarbon products are represented by eq 111. Additionally, the amounts of H₂ consumed and H₂O produced are calculated according to the stoichiometric reactions for each hydrocarbon species (eq 6), and their output flow rates can be obtained.

### Example 1.19

### Hydrocarbon Upgrading Units

It is crucial to upgrade the FT effluent to fuel-grade hydrocarbons for resale to the transportation sector. The process layout follows from a Bechtel (Bechtel, 1998; Bechtel, 1992, which are incorporated herein by reference as if fully set forth) design and includes a hydrocarbon recovery unit, a wax hydrocracker, a distillate hydrotreater, a kerosene hydrotreater, a naphtha hydrotreater, a naphtha reformer, a C₄ isomerizer, a C₅/C₆ isomerizer, a C₃/C₄/C₅ alkylation unit, and a saturated gas plant (FIG. 21). Although a kerosene hydrotreater is not provided in the Bechtel design, it is assumed that the distribution of the input carbon to kerosene and light gases is exactly the same as the distillate hydrotreater. Operating conditions were not reported from Bechtel; therefore, to determine the output, the appropriate mass balances for the baseline Illinois No. 6 coal case study were used (Bechtel, 1993, which is incorporated herein by reference as if fully set forth). That is, for each upgrading unit, the distribution of the input carbon is determined to either exactly match or closely approximate the distribution reported by Bechtel. The wax hydrocracker, distillate hydrotreater, naphtha hydrotreater, C₅/C₆ isomerizer, and C₄ isomerizer all require an input of hydrogen. After distributing all input oxygen as the wastewater stream, the effluent of each upgrading unit can be set to exactly match the Bechtel output by adjusting the flow of hydrogen. Given the mass outputs of the case study (see Table 16), the distribution of the input carbon can be calculated. The following equations (eqs 112-119) define the operation of the wax hydrocracker unit (P402) and are presented as an example for the calculation of all other upgrading units.

$$\begin{matrix}{\sum }_{s ∈ {S}_{401 , WX}} ( {AR}_{C , s} {F}_{s}^{401 , WX} ) = {F}_{C}^{402} & ( 112 ) \\ {\sum }_{s ∈ {S}_{401 , WX}} ( {AR}_{O , s} {F}_{s}^{401 , WX} ) = {F}_{O}^{402} & ( 113 ) \\ {hr}_{C}^{402} {F}_{C}^{402} + {hr}_{O}^{402} {F}_{O}^{402} - {\sum }_{s ∈ {S}_{401 , WX}} ( {AR}_{H , s} {F}_{s}^{401 , WX} ) = {F}_{H}^{402} & ( 114 ) \\ {cf}_{s}^{402} {F}_{C}^{402} = {AR}_{C , s} {F}_{s}^{402 , LG} ∀ s ∈ {S}_{402 , LG} & ( 115 ) \\ {cf}_{s}^{402} {F}_{C}^{402} = {AR}_{C , s} {F}_{s}^{402 , N} ∀ s ∈ {S}_{402 , N} & ( 116 ) \\ {cf}_{s}^{402} {F}_{C}^{402} = {AR}_{C , s} {F}_{s}^{402 , C 56} ∀ s ∈ {S}_{402 , C 56} & ( 117 ) \\ {cf}_{s}^{402} {F}_{C}^{402} = {AR}_{C , s} {F}_{s}^{402 , D} ∀ s ∈ {S}_{402 , D} & ( 118 ) \\ {F}_{O}^{402} = {F}_{{H}_{2} O}^{402 , WW} & ( 119 )\end{matrix}$$

TABLE 16
Bechtel Illinois No. 6 Coal Case Study Output Flow Rates
for Units That Consume Hydrogen⁶⁴

| | Output Flow (lb/h) | | | | |
|---|---|---|---|---|---|
| | Hydrocracker | Hydrotreater | | Isomerizer | |
| component | wax | distillate | naphtha | C₅/C₆ | C₄ |

Light Gases

| | | | | | |
|---|---|---|---|---|---|
| CH₄ | 141 | 85 | 350 | 49 | 92 |
| C₂H₆ | 141 | 128 | 1342 | 16 | 207 |
| C₃H₈ | 4187 | 298 | 1711 | 641 | 561 |
| iC₄H₁₀ | 5546 | 128 | 240 | 299 | 0 |
| nC₄H₁₀ | 4500 | 213 | 1144 | 0 | 0 |

C₅₋₆Gases

| | | | | | |
|---|---|---|---|---|---|
| iC₅H₁₂ | 6903 | 0 | 75 | 0 | 0 |
| nC₅H₁₂ | 5830 | 0 | 3572 | 0 | 0 |
| iC₆H₁₄ | 10978 | 0 | 2013 | 0 | 0 |
| nC₆H₁₄ | 6734 | 0 | 18119 | 0 | 0 |

Isomerate

| | | | | | |
|---|---|---|---|---|---|
| iC₄H₁₀ | 0 | 0 | 0 | 0 | 46358 |
| nC₄H₁₀ | 0 | 0 | 0 | 0 | 1929 |
| iC₅H₁₂ | 0 | 0 | 0 | 16100 | 0 |
| iC₆H₁₄ | 0 | 0 | 0 | 37196 | 0 |

Gasoline

| | | | | | |
|---|---|---|---|---|---|
| C₇H₁₆ | 0 | 0 | 0 | 0 | 0 |
| C₈H₁₈ | 0 | 0 | 0 | 0 | 0 |
| C₉H₂₀ | 54129 | 0 | 70456 | 0 | 0 |
| C₁₅H₃₂ | 187692 | 90520 | 0 | 0 | 0 |

where AR_{C,s}, AR_{O,s}, and AR_{H,s} are the atomic ratios of carbon, oxygen, and hydrogen in compound s, respectively; Fₛ^{WX} is the molar flow rate of compound s in the wax substream (WX) from the hydrocarbon recovery unit (P401); and F_{C} and F_{O} are the total atomic input flow rates for carbon and oxygen to the upgrading unit; F_{H} is the additional hydrogen that must be input to the upgrading unit to match the Bechtel output; hr_{C} and hr_{O} are the hydrogen ratios in compounds containing carbon and oxygen, respectively; and cfₛ are the carbon fractions in compound s of the output streams obtained from the Bechtel case study (see Table 16). Equations 112-114 calculate the total incoming atomic flow rates into the unit, eq 119 sends all the oxygen into the wastewater stream (WW), and eqs 115-118 define the output composition in each substream existing the unit. The mass balances for all other upgrading units are completed similar to that for this hydrocracker unit.

### Example 1.20

### Steady-State Process Simulation

Steady-state process simulations on seven process alternatives are completed to study the efficiency of the proposed hybrid system. The feedstock is either (i) coal only (C), (ii) biomass only (B), or (iii) a hybrid combination of coal, biomass, and natural gas (H). Hydrogen is obtained either from SRM purchase (R) or via electrolysis (E), and light gases are reformed either by an ATR (A) or combusted using a gas turbine engine (T).

The seven combinations are as follows: C-R-A, C-E-A, B-R-A, B-E-A, H-R-A, H-E-A, and H-R-T. The feedstocks are normalized to a total of 2000 tonnes/day, as presented in Table 17.

TABLE 17
Simulation Results and Analysis for Seven Process Alternatives

| | | | | | | |
|---|---|---|---|---|---|---|
| C-R-A | C-E-A | B-R-A | B-E-A | H-R-A | H-E-A | H-R-T |

Feedstocks (TPD^{a})

| | | | | | | | |
|---|---|---|---|---|---|---|---|
| biomass | 0 | 0 | 2000 | 2000 | 948.62 | 948.62 | 948.62 |
| coal | 2000 | 2000 | 0 | 0 | 678.87 | 678.87 | 678.87 |
| natural gas | 0 | 0 | 0 | 0 | 372.51 | 372.51 | 372.51 |
| hydrogen | 347 | 0 | 275 | 0 | 298 | 0 | 254 |
| butanes | 21.9 | 21.9 | 19.3 | 19.3 | 20.1 | 20.1 | 19.2 |
| process water | 260.06 | 882.14 | 291.17 | 883.87 | 247.97 | 861.41 | 15.55 |

Products (TBD^{b})

| | | | | | | | |
|---|---|---|---|---|---|---|---|
| oxygen (TPD) | 0 | 1041 | 0 | 825 | 0 | 894 | 0 |
| propanes | 0.22 | 0.22 | 0.20 | 0.20 | 0.20 | 0.20 | 0.18 |
| gasoline | 7.57 | 7.57 | 4.66 | 4.66 | 6.85 | 6.85 | 6.27 |
| diesel | 2.46 | 2.56 | 1.51 | 1.51 | 2.22 | 2.22 | 2.04 |
| kerosene | 1.32 | 1.32 | 0.82 | 0.82 | 1.20 | 1.20 | 1.10 |
| energy efficiency (LHV^{c}) | 69.50% | 70.80% | 65.20% | 60.10% | 65.70% | 61.90% | 68.10% |
| number of plants needed | 1163 | 1163 | 1888 | 1888 | 1285 | 1285 | 1403 |
| % C vented | 0.51% | 0.46% | 0.38% | 0.31% | 0.42% | 0.39% | 9.80% |
| biomass demand (MTPY^{d}) | 0 | 0 | 1,176 | 1,176 | 379 | 379 | 414 |

^{a}TPD = metric tons per day.
^{b}TBD = thousand barrels per day.
^{c}LHV = lower heating value.
^{d}MTPY = million short tons per year.

Because the consumption of liquid fuels has decreased in recent months, but is expected to rise in the coming years, the 2010 demand is estimated based on the reported 2008 data. Therefore, the target demand for the CBGTL process are 8803 thousand barrels per day (TBD) of gasoline, 2858 TBD of diesel, and 1539 TBD of kerosene. More plants are required for runs with increasing amounts of biomass feedstock, because of its lower carbon content, in comparison to coal. Current total biomass availability in the Unites States is 416 million dry tons per year (MTPY), corresponding to ˜35 vol % of transportation fuel. Clearly, the pure biomass feedstock requires significantly more production than is currently available, but the total annual

production of 1.144 MTPY is not far above the feasibility target of the U.S. Department of Energy (DOE). The hybrid system allows for biomass to be directly integrated into a FT process to satisfy all transportation demand using what feedstock is available. The number of plants needed in Table 17 represents the total number of CBGTL processes required to satisfy the entire transportation demand. A smaller number of plants would be required if the results of the case studies are scaled up to use a larger feedstock quantity. The scale up is likely to be limited by the input quantity of the biomass, because it is the most expensive feedstock to transport.

A result is the small amount of carbon vented from the system. Almost all studied processes only vent between 0.31%-0.51% of the feed carbon, with the gas turbine system venting 9.8% of the carbon, because of the pure air stream being fed to the turbine combuster. The recovery of CO₂ that will be recycled back into the process for the gas turbine case is also limited by the specification of the Rectisol unit (3 mol % CO₂ in the vented stream). With the exception of the gas turbine system, these numbers are an order of magnitude lower than those recently reported for similar Fischer-Tropsch systems. If an oxygen-blown gas turbine is utilized, the vented carbon could theoretically be reduced to the levels of the other simulations. It is critical to note that none of these cases have required sequestration of CO₂, so all of the carbon that is notvented is converted directly to the desired transportation fuels, with the exception of a small amount of C₃ propanes that are extracted from the saturated gas plant. In this case study, the propanes are sold as a byproduct, although they could have been sent to the ATR or gas turbine, along with the other light gases (see FIG. 22).

### Example 1.21

### Economic Analysis

Once each of the seven process alternatives has been fully heat- and power-integrated using the framework presented in Example 2, a detailed economic analysis is performed to determine the crude oil price that makes the CBGTL process competitive with current petroleum-based processes. The total permanent investment is first calculated either using the Aspen Process Economic Analyzer or from cost estimates from the literature. The utility costs are included from the heat and power integration model, and all feedstock costs are taken from recent projections. The refinery margin (RM) is used to calculate the product costs for a given crude oil price and the break-even oil price (BEOP) is calculated by setting the net present value of the plant equal to zero. Details for all calculations including cost estimates and economic assumptions are provided below.

### Example 1.22

### Capital Cost Assumptions

The direct permanent investment (DPI) of all pumps, compressors, turbines, and flash units is calculated using the Aspen Process Economic Analyzer, while the DPI of the remaining process units is calculated using estimates from several data sources,7,11,27,28,31 using the cost parameters in Table 18 and eq 120.

$$DPI = ( 1 + BOP ) {{C}_{0} ( \frac{S}{{S}_{0}} )}^{sf} {n}^{0.9} \qquad ( 120 )$$

where C₀ is the base cost, S₀ is the base capacity, S is the actual capacity, n is the total number of trains, sf is the cost scaling factor, and BOP is the balance of plant percentage (e.g., site preparation, utility plants, etc.). The BOP value is calculated for the FT units, the hydrocarbon recovery unit, and all upgrading units, as a function of the feedstock higher heating value (HHV),11 using eq 121.

$$BOP ( \% ) = \frac{0.8867}{{MW}_{HHV}^{0.2096}} \qquad ( 121 )$$

The BOP value is either assumed to be 15.5% or included in the base cost for the remaining process units. All results are expressed in 2010 dollars, using the Chemical Engineering Plant Cost Index65 and the GDP inflation index2 to convert the original price when applicable.

TABLE 18
Process Flowsheet Reference Capacities, Costs (2010 $), and Scaling Factors

| unit name | C₀ (MMS) | S₀ | Sₘₐₓ | units | scale basis | sf | BOP | ref |
|---|---|---|---|---|---|---|---|---|
| biomass H&D^{a} | $27.04 | 2000 | N/A | TPD^{b} | dry biomass | 0.67 | included | 31 |
| biomass gasifier | $151.43 | 815 | 568 | MW LHV | dry biomass | 0.67 | 15.50% | 11 |
| coal H&D^{a} | $79.41 | 2464 | 2616 | TPD | dry coal | 0.6 | included | 27 |
| coal gasifier | $132.46 | 2464 | 2722 | TPD | dry coal | 0.6 | included | 27 |
| RWGS | $3.05 | 2556 | 2600 | TPD | output | 0.65 | 15.50% | 27 |
| COS hydrolysis | $2.97 | 4975 | 7500 | TPD | ouput | 0.67 | 15.50% | 27 |
| acid gas recccvery | $52.58 | 200000 | 700000 | Nm³/h | output | 0.63 | 15.50% | 11 |
| Fischer-Tropsch | $39.59 | 226669 | 228029 | Nm³/h | feed | 0.75 | 25.69% | 28 |
| hydrocarbon rec. | $0.73 | 152.32 | 2176 | TPD | feed | 0.7 | 25.69% | 28 |
| wax hydrocracker | $9.35 | 97.92 | 6256 | TPD | feed | 0.55 | 25.69% | 28 |
| dist. hydrotreater | $2.50 | 31.55 | 7072 | TPD | feed | 0.6 | 25.69% | 28 |
| nap. hydrotreater | $0.76 | 22.85 | 7072 | TPD | feed | 0.65 | 25.69% | 28 |
| ker. hydrotreater | $2.50 | 31.55 | 7071 | TPD | feed | 0.6 | 25.69% | 28 |
| nap. reformer | $5.21 | 36.99 | 8160 | TPD | feed | 0.6 | 25.69% | 28 |
| C₅/C₆ isomerizer | $0.96 | 13.06 | 2720 | TPD | feed | 0.62 | 25.69% | 28 |
| C₄ isomerizer | $10.72 | 560.06 | N/A | TPD | feed | 0.6 | 25.69% | 29 |
| C₃/C₄/C₅ alkylation | $59.00 | 1102.83 | N/A | TPD | feed | 0.6 | 25.69% | 29 |
| saturated gas plant | $8.84 | 6118 | N/A | TPD | output, gas, die | 0.6 | 25.69% | 29 |
| ATR | $3.18 | 430639 | 9438667 | Nm³/h | output | 0.67 | included | 11 |
| ASU | $55.95 | 1839 | 2500 | TPD | oxygen outlet | 0.5 | 15.50% | 11 |
| Claus plant | $23.41 | 135 | N/A | TPD | sulfur | 0.67 | 15.50% | 27 |

^{a}H&D = handling and drying.
^{b}TPD = metric tons per day.

### Example 1.23

### Feedstock and Product Assumptions

The price (“asreceived”, delivered to plant gate, 2010 $) of herbaceous biomass, Illinois No. 6 coal, and natural gas is $5.26/GJ HHV,11 $42.16/short ton, and $7.48/103 ft³, respectively (see Table 19). Disposal costs of wastewater and ash are included in the operating and maintenance costs of the process units producing those wastes. The utility costs for each process alternative are taken directly from the results of the heat and power integration minimum utility model presented in Example 2. Because of the variable marketability of sulfur, no credit is taken for the sale as a byproduct.

The resale cost of the transportation fuels is based on the price of crude oil and the RM for each product. The RM is the difference between the sale price of petroleum products and the purchase price of crude oil and is estimated as the 1992-2003 average,1 after adjustment with the U.S. Gross Domestic Index. The RM for gasoline, diesel, and kerosene is $0.333/gal, $ 0.266/gal, and $0.217/gal, respectively (see Table 19). The RM for diesel is $0.05/gal higher than the average, because of the estimated additional cost for the production of low-sulfur diesel.

### Example 1.24

### Additional Economic Assumptions

Table 20 lists the additional economic assumptions. The total depreciable capital (TDC) is the sum of the DPI plus general and administrative (G&A) capital overhead and contract fees, each of which is estimated to be 3% of the DPI. The total permanent investment (TPI) is the sum of the TDC plus the capital contingencies, which is estimated to be 18% of the TDC. The distribution of the TPI over the three-year construction/startup period is ¼ in the first year, ½ in the second, and ¼ in the third. The working capital is estimated to be 5% of the TPI, to be used during startup in the third year of the plant life. The book life of the plant is taken to be 30 years, with a yearly operating capacity of 8000 h. The salvage value of the plant is estimated to be 20% of the TPI.

All operating costs are also presented in Table 20. The annual maintenance costs are taken as 4% of the TPI, the labor costs (10 operators, 1 supervisor) are $350/h, and the operating charges are assumed to be 25% of the labor cost. The summation of these three items is termed the operating labor and maintenance (OL&M) costs. The subtotal operating cost (SOC) is defined as the sum of the raw materials, utilities, and OL&Mcosts. The G&A operating expenses are estimated to be 8% of the SOC, and the plant overhead is estimated to be 50% of the OL&M. The total operating costs is then calculated as the sum of the SOC, the G&A operating expenses, and the plant overhead.

### Example 1.25

### Break-Even Oil Price

Based on the aforementioned assumptions, the net present value (NPV) of the CBGTL process can be calculated for any given crude oil price (COP). For each year y in the economic life of the plant, the sales, S_{y}, can be calculated as the sum of the three major transportation fuel product sales plus the sale of byproduct propane (eq 124). The product fuels sales are adjusted for the appropriate year using the escalation factor, P_{Esc}. Note that the sales will be equal to zero during the first three years of the plant life (y_{St}) 3), because of construction time and startup (see Table 20).

PR_{y}(1+P_{Esc})^{y}[F_{Gas}(COP+RM_{G})+F_{Die}(COP+RM_{D})+Fₖₑᵣ(COP+RM_{K})] (122)

BY_{y}=(1+P_{Esc})^{y}Cost_{Pro}F_{Pro} (123)

S_{y}=PR_{y}+BY_{y} (124)

The total permanent investment (TPI) is distributed during construction time using the distribution factor f_{y}^{Cap}. During plant construction, we have f₁^{Cap}) 0.25, f₂^{Cap}) 0.5, and f₃^{Cap}) 0.25. The working capital, WC_{y}, is defined as 5% of the TPI and is only utilized during startup in year 3. The 20% salvage value of the plant, SV_{y}, is taken into account at the end of the economic life of the plant (yEnd=30). The raw material cost is calculated using the flow rate of biomass, coal, natural gas, butanes, and hydrogen (eq 126) and is escalated using R_{Esc}. The utility cost is calculated based on the amount of cooling water and electricity needed for the process (eq 127). Note that the electrolyzer-based processes will not require hydrogen. The yearly operating costs, OP_{y}, can be calculated using the raw materials, utilities, operating labor and maintenance, operating charges, plant overhead, and G&A costs (see Table 20), as outlined above. The operating labor and maintenance costs will be escalated using the appropriate factor.

$$\begin{matrix} {CAP}_{y} = {( 1 + {C}_{Esc} )}^{y} {f}_{y}^{Cap} TPI + {WC}_{y} - {SV}_{y} & ( 125 ) \\ {RM}_{y} = {( 1 + {R}_{Esc} )}^{y} ( {Cost}_{Bio} {F}_{Bio} + {Cost}_{Coal} {F}_{Coal} + {Cost}_{NG} {F}_{NG} + {Cost}_{Hyd} {F}_{Hyd} + {Cost}_{But} {F}_{But} ) & ( 126 ) \\ {U}_{y} = {( 1 + {U}_{Esc} )}^{y} ( {Cost}_{CW} {F}_{CW} + {Cost}_{EI} + {F}_{EI} ) & ( 127 ) \\ {CF}_{y} = ( {S}_{y} - {OP}_{y} ) ( 1 - TR ) - ( TR ) {DEP}_{y} - {CAP}_{y} & ( 128 ) \\ NPV = {\sum }_{y \leq {y}_{End}} \frac{{S}_{y}}{{( 1 + RR )}^{y}} & ( 129 )\end{matrix}$$

TABLE 20
Additional Economic Assumptions

| parameter | value | parameter | value |
|---|---|---|---|
| G&A capital overhead | 3% | plant overhead (% of | 50% |
| (% of DPI) | | OL&M Costs) | |
| contract fees (% of DPI) | 3% | G&A operating | 8% |
| | | expenses (% of SOC) | |
| capital contingencies | 18% | yearly operating | 8000 h |
| (% of TDC) | | capacity | |
| working capital (% of | 5% | tax rate | 40% |
| TPI) | | | |
| construction time | 2.5 yrs | desired rate of return | 15% |
| startup time | 0.5 yrs | salvage value (% of | 20% |
| | | TPI) | |
| book life and economic | 30 yrs | products escalation | 1% per yr |
| life of investment | | | |
| maintenance costs (% of | 4% | raw material escalation | 1% per yr |
| TPI) | | | |
| labor costs ($/h; all | $300, $50 | utilities escalation | 1% per yr |
| operators, supervisor) | | | |
| operating charges (% of | 25% | | |
| labor costs) | | | |

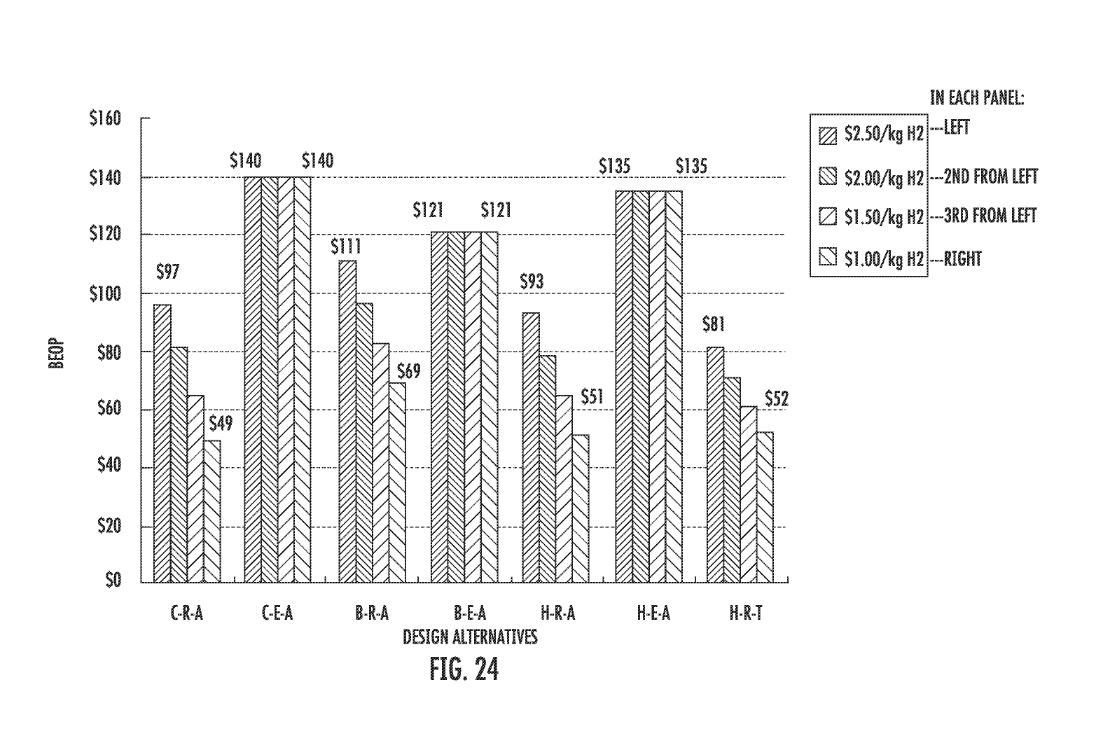

Using a straight-line depreciation method over 10 years and a tax rate (TR) of 40%, the cash flow for a given operating year is defined in eq 128. The NPV of the plant is then calculated by summing the discounted cash flows over the entire economic life of the plant, using the desired rate of return (RR) (see eq 129). Upon completion of a process simulation and the simultaneous heat and power integration, all of the information in eqs 124-129 is known, except for the crude oil price (COP). The break-even oil price (BEOP) is defined as the crude oil price for which the NPV of the process is equal to zero. Since the RM is used to calculate the selling price of the transportation fuels, this metric is considered the price of crude oil at which the CBGTL process is economically competitive with petroleumbased processes. The variability in the BEOP, with respect to hydrogen, is presented in Table 21 and graphically in FIG. 24. Hydrogen prices greatly influence the competitiveness of the process because of the high requirement of hydrogen input to the system. Processes with electrolysis are not affected by the price changes since hydrogen is produced on-site. Their high BEOP is due to the high capital cost of electrolyzer and the price of electricity. For the other cases, the hybrid processes are more competitive than the coal-only or biomass-only cases at almost all hydrogen price values. At $1.25/kg H₂ and lower, the coal process also becomes competitive with a BEOP of $57 and $49. At hydrogen prices above $1.00/kg H₂, the gas turbine case is more competitive than the other cases. However, this process is also associated with higher CO₂ emission, as discussed previously. Overall, the results show that fuel products from this process can be competitive with petroleum-based fuels, highlighting the important benefits such as near 100% carbon conversion and no CO₂ sequestration required.

TABLE 21
Break-even Oil Price (BEOP) of Seven
Process Alternatives Using Distinct Hydrogen Prices^{a}

| hydro- | | | | | | | |
|---|---|---|---|---|---|---|---|
| gen | | | | | | | |
| price | BEOP | | | | | | |
| ($/kg) | C-R-A | C-E-A | B-R-A | B-E-A | H-R-A | H-E-A | H-R-T |
| $2.50 | $97 | $140 | $111 | $121 | $93 | $135 | $81 |
| $2.00 | $89 | $140 | $104 | $121 | $86 | $135 | $76 |
| $2.00 | $81 | $140 | $97 | $121 | $79 | $135 | $71 |
| $1.75 | $73 | $140 | $90 | $121 | $72 | $135 | $66 |
| $1.50 | $65 | $140 | $83 | $121 | $65 | $135 | $61 |
| $1.25 | $57 | $140 | $76 | $121 | $58 | $135 | $57 |
| $1.00 | $49 | $140 | $69 | $121 | $51 | $135 | $52 |

^{a}Electricity price = $0.0775/kWh; electrolyzer cost = $1000/kW.

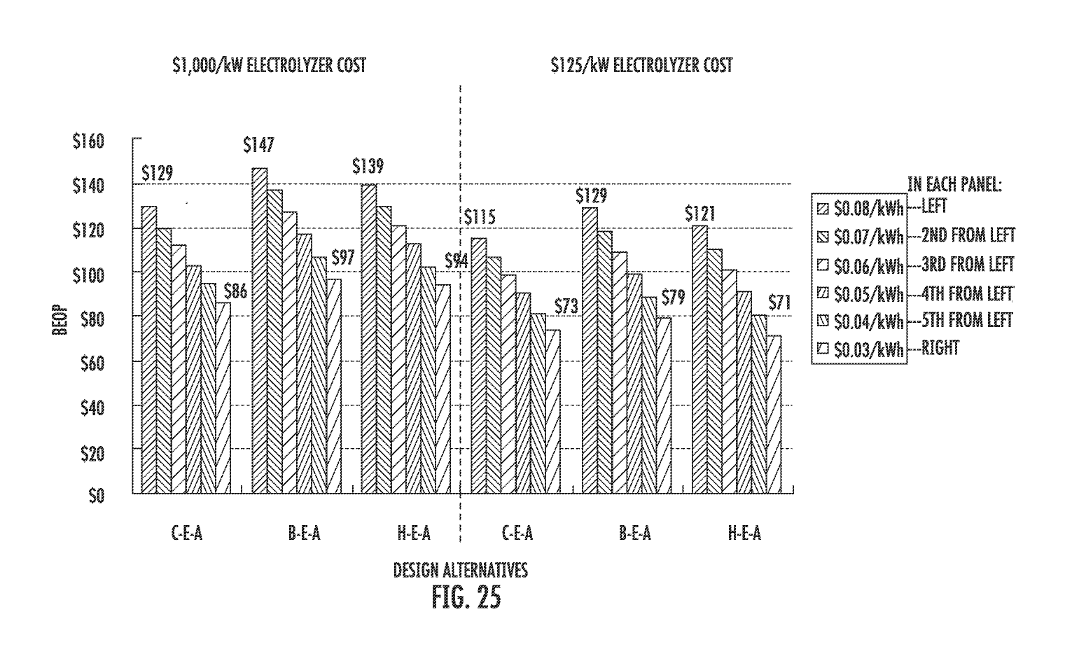

The economics of the electrolysis-based processes are analyzed with respect to changes in electricity prices and electrolyzer capital cost. Table 22 shows that a reduction in electricity prices from $0.08/kWh to $0.03/kWh is needed for the electrolysis-based processes to be competitive, with respect to ATR or gas-turbine-based processes at $2.00/kg H₂. If the electrolyzer cost is further reduced to $125/kW at $0.03/kWh, the electrolysis-based processes become the most competitive alternative. (Also see FIG. 25). Thus, as electrolyzer technologies develop in the future and as electricity price decreases, electrolysis as the hydrogen-producing option will become more attractive.

TABLE 22
Break-even Oil Price (BEOP) Using Distinct
Electricity Prices and Electrolyzer Capital Costs

| | BEOP | | | | | |
|---|---|---|---|---|---|---|
| | Electrolyzer | | | Electrolyzer | | |
| electricity | Cost = $1000/kW | | | Cost = $125/kW | | |
| price ($/kWh) | C-E-A | B-E-A | H-E-A | C-E-A | B-E-A | H-E-A |
| $0.08 | $129 | $147 | $139 | $115 | $129 | $121 |
| $0.07 | $120 | $137 | $130 | $107 | $119 | $111 |
| $0.06 | $112 | $127 | $121 | $98 | $109 | $101 |
| $0.05 | $103 | $117 | $112 | $90 | $99 | $91 |
| $0.04 | $95 | $117 | $103 | $81 | $89 | $81 |
| $0.03 | $86 | $97 | $94 | $73 | $79 | $71 |

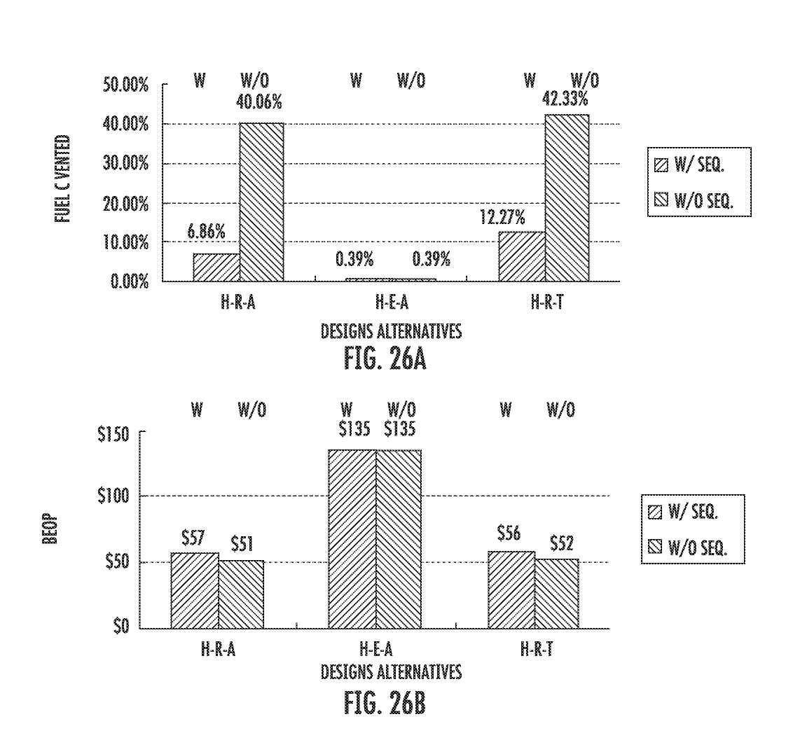

Hybrid processes with steam reforming of methane (SRM) with and without CO₂ sequestration are assessed in terms of the BEOP and the total emitted carbon in Table 23. The total vented carbon is the sum of carbon emitted from the process and the carbon emitted from the steam reforming of methane to produce hydrogen. Based on the figures reported by the National Research Council, 2004, which is incorporated herein by reference as if fully set forth, the CO₂ emission from SRM technology is 1.53 kg/kg H₂ with sequestration and 9.22 kg/kg H₂ without sequestration, and the corresponding hydrogen prices are $1.22/kg and $1.03/kg, respectively. The total CO₂ emission is then calculated, and the results are displayed in Table 23. It is shown that the CBGTL processes that consume hydrogen from SRM give rise to a higher percentage of vented carbon, with respect to the total fuel carbon (i.e., CBGTL feedstock and natural gas feedstock to produce hydrogen in the steam reforming process). Carbon sequestration is needed for the stream reforming process to reduce the amount of vented carbon. FIG. 26 shows that, with a slight increase in the BEOP using CO₂ sequestration, a significant reduction in carbon emission is achieved. The tradeoff between BEOP and carbon emission is even more marked when comparing the two technology alternatives for hydrogen production. With a substantial increase in the BEOP from the H-R-A and H-R-T cases to the H-E-A case, a very low carbon emission can be achieved.

TABLE 23
Comparison of Hydrogen Sources and the Total Carbon
Emissions from the CBGTL Process

| | H-R-A | H-E-A | H-R-T |
|---|---|---|---|
| hydrogen needed (kg/yr) | 9.93 × 10⁷ | | 8.47 × 10⁷ |
| CO₂ vented from | 5.82 × 10⁶ | 5.40 × 10⁶ | 1.36 × 10⁸ |
| CBGTL (kg/yr) | | | |
| SRM CO₂ vented | 1.52 × 10⁸ | | 1.30 × 10⁸ |
| w/sequestration (kg/yr) | | | |
| SRM CO₂ vented | 9.16 × 10⁸ | | 7.81 × 10⁸ |
| w/o sequestration (kg/yr) | | | |
| % fuel C vented | 6.86 | 0.39 | 12.27 |
| w/sequestration | | | |
| % fuel C vented | 40.06 | 0.39 | 42.33 |
| w/o sequestration | | | |
| BEOP w/sequestration | $57 | $135 | $56 |
| BEOP w/o sequestration | $51 | $135 | $52 |

A novel coal, biomass, and natural gas to liquids (CBGTL) process that produces transportation fuels from coal, biomass, and natural gas is introduced and is shown to possess capabilities of converting almost 100% of the feedstock carbon using a reverse water-gas-shift reactor. Key components of the process include the gasification of coal and biomass feedstock, syngas treatment, hydrocarbon production and upgrading, and hydrogen generation. Stoichiometric-based mathematical models that predict the output syngas composition of coal and biomass gasifiers are developed and integrated into the process simulation. Results from seven process alternatives considered above show that the hybrid process has the potential to satisfy the U.S. transportation demand with very low carbon loss, eliminating the need for CO₂ sequestration if hydrogen can be generated from a noncarbon source.

The economic analysis for the CBGTL processes provides the price of crude oil for which the processes become competitive with current petroleum-based systems. A total permanent investment was calculated using both the Aspen Process Economic Analyzer and cost estimates from several literature sources. Along with the appropriate product sales, raw material costs, operating labor and maintenance costs, depreciation, and other economic factors, the net present value of the CBGTL process is calculated as a function of the crude oil price. The break-even oil price is strongly dependent on the selling price of hydrogen, but it is equal to $56/barrel for the hybrid process (H-R-A) if steam reforming of methane is utilized and generally ranges from $51/barrel to $79/barrel for hydrogen prices between $1.00/kg and $2.00/kg.

### Example 2

### Simultaneous Heat and Power Integration

This example presents an approach for the generation of a novel heat exchange and power recovery network (HEPN) for use with any large-scale process. A three-stage decomposition framework is introduced to sequentially determine the minimum hot/cold/power utility requirement, the minimum number of heat exchanger matches, and the minimum annualized cost of heat exchange. A superset of heat engine operating conditions is used to derive the heat engine design alternatives that produce the maximum amount of electricity that can be generated when there is complete integration with the process streams. Given the minimum utility loads and the appropriate subnetworks for each process flowsheet, the minimum number of heat exchanger matches is found for each subnetwork. Weighted matches and vertical heat transfer are used to distinguish among the heat exchanger sets, to postulate the appropriate set of matches that will yield the lower minimum annualized cost. Finally, a minimum annualized cost model was presented, which uses Aspen Plus process information to estimate the cost functions for a heat exchanger match and the overall heat transfer coefficient. The proposed model is then used to analyze the seven simulated process flowsheets detailed in Example 1. Detailed case studies are presented for the three hybrid process flowsheets to highlight the key differences in the HEPN for each process.

Example 1 detailed the design of the coal, biomass, and natural gas to liquids (CBGTL) process, including a complete process description and the novel biomass and coal gasifier models used to determine the composition of the generated syngas. Seven process alternatives were considered that varied with regard to the choice of feedstock composition, the hydrogen production, and the treatment of the light hydrocarbon recycle stream.

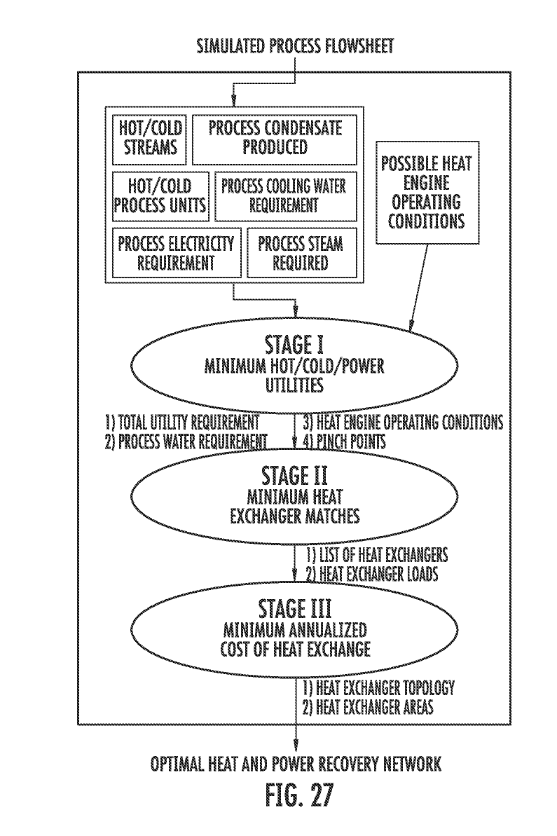

The mathematical models used to fully develop the heat exchanger and power recovery network (HEPN) for the seven CBGTL process flowsheets is presented in this example. Given the information provided by the process flowsheet, the goals of the mathematical model are to determine (a) the hot, cold, and power utility loads; (b) the heat exchanger matches; (c) the areas of each match; and (d) the topology of the heat exchanger network. This can either be achieved through a decomposition of the tasks into subtasks or through a simultaneous consideration of all goals. Although approaches for the synthesis of heat exchanger networks without decomposition have been developed, the simultaneous heat and power integration problem via a decomposition framework into three tasks (FIG. 27) is disclosed to, first, (I) minimize the total hot/cold/power utility requirement, then (II) minimize the heat exchanger matches to meet the given utility requirement, and finally (III) determine the topology of heat exchangers given the matches, which provides the minimum annualized cost. The model for part (I) incorporates heat engines to optimally produce electricity from steam turbines while fully integrating all of the hot and cold process streams and process units in a heat exchange and power recovery network. The optimal solution of part (I) will provide the appropriate pinch points of the system and will decompose the process streams into subnetworks. A strict pinch criterion) is assumed for part (II), so that no heat transfer occurs between the subnetworks during parts (II) and (III). This allows the subnetworks in parts (II) and (III) to be analyzed individually, reducing the complexity of each mathematical model.

The following Examples describe each subtask. Examples 2.1-2.3 discuss a novel mathematical model to simultaneously minimize both the cost of the hot/cold utilities (i.e., steam and cooling water) and the power utilities (i.e., electricity). This is accomplished by postulating a series of heat engines with given steam turbine operating conditions, so that heat can be transferred directly from the process flowsheet to the heat engines. Examples 2.4-2.9 discuss the model used to find the minimum number of heat exchangers that are necessary to provide the minimum utility requirements for the process flowsheet. Vertical heat transfer and weighted matches are used to distinguish between solutions with the same value. Finally, Examples 2.10-2.17 describe the model used to determine the appropriate topology of the heat exchanger matches. Appropriate cost functions are defined for each individual heat exchanger match taking into account both the assumed effect of pressure and stream flow rate on the annualized cost and the overall heat transfer coefficient. Overall results for each of the seven process flowsheets will be presented in the Examples. Further detailed illustrative examples are presented for the three hybrid flowsheets H-RA, H-E-A, and H-R-T for Examples 2.4-2.9 and “Examples 2.10-2.1 to show the proper topology for one representative subnetwork.

### Example 2.1

### Minimum Hot/Cold/Power Utilities

The waste heat streams from the processes can either provide steam or generate electricity using a HEPN that consists of heat exchangers, water boilers, heat engines, and heat pumps. A model for the minimum hot/cold/power utility cost was proposed using heat engines and pumps to provide the electricity to be generated by the hot and cold process streams. However, this model is only capable of providing target utility usage, since the electricity produced or used by the process streams is assumed to be equal to the Carnot efficiency of the engine or pump. These targets will not be attainable, because of the limitations on the efficiency of the turbine in the heat engine and the compressor in the heat pump. A further assumption of the model is the splitting of the process streams, such that one fraction operates entirely in the process heat exchanger network (i.e., hot and cold process streams, hot and cold utilities) while the remaining fraction operates entirely in the heat engines or pumps (i.e., condensers and boilers of the working fluid). Such a discretization at the global level may lead to a suboptimal hot/cold/power utility cost, since the HEPN may require distinct fractions that interact with the heat engines/heat pumps at distinct temperature intervals.

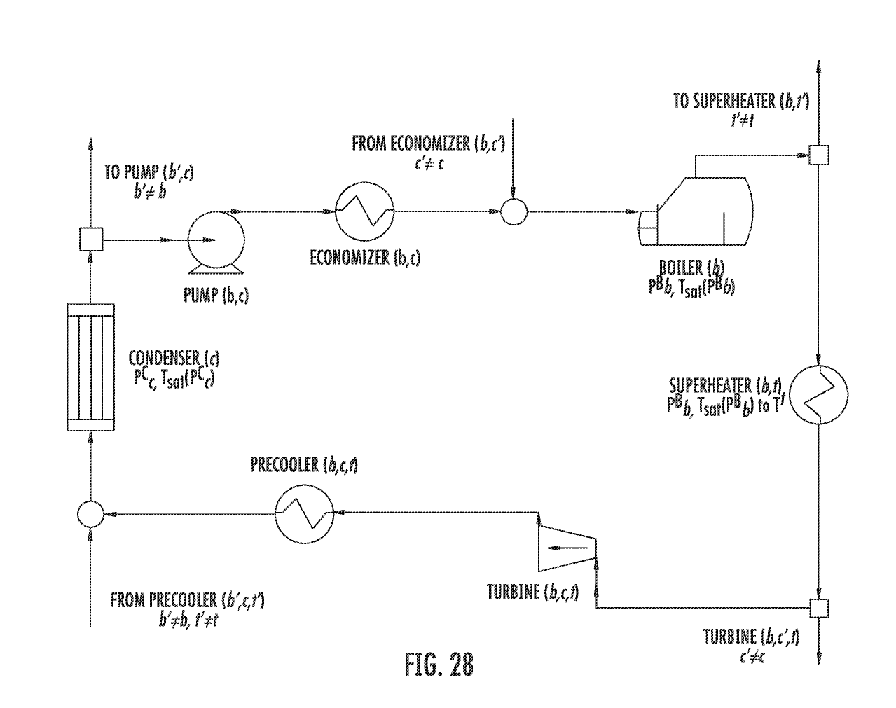

To address this issue, the minimum hot/cold/power utility model is expanded by postulating a set of heat engines that provide the necessary electricity. The conditions of the turbines and pumps are known a priori, so the electricity delivered may be directly calculated for a particular heat engine by specifying the isentropic and mechanical efficiency. Specifically, discrete sets of boiler pressures (P_{b}^{B}), condenser pressures (P_{c}^{C}), and turbine inlet temperatures (Tₜ) are selected that define a finite amount of heat engines (FIG. 28). For each boiler, condenser, and turbine triplet, denoted as (b, c, t), five heat exchangers are defined including (1) an economizer, (2) an evaporator, (3) a superheater, (4) a precooler, and (5) a condenser. The economizer, evaporator, and superheater are designed to heat up the pump outlet to the turbine inlet temperature while the precooler and condenser will decrease the turbine outlet temperature to the pump inlet temperature. The heat exchangers are discretized to operate in regions of sensible and latent heat transfer, because of the varying annualized costs associated with heat transfer involving a phase change. That is, a kettle vaporizer will be used to model the evaporator while floating head units model the other exchangers. Furthermore, the convective heat transfer coefficient is different for the pure vapor, pure liquid, and mixed vapor-liquid units. Hence, the annualized cost function is different for each of the five heat exchangers used in the heat engine. Although these costs are not directly included until the third stage of the HEPN decomposition, the discretization of the heat exchangers at this stage allows for the proper calculation of the sensible and latent heat without introducing additional constraints to the minimum hot/cold/power utility or minimum matches model. Note that heat pumps are not necessary for the CBGTL process, because of the large amount of waste heat provided by the process streams. However, this methodology could be expanded by also postulating a set of heat pumps.

A discrete set of heat engines is selected using a superset of possible operating conditions (FIG. 28). The condenser is allowed to operate at either 1, 5, 15, or 40 bar, the boiler operates at either 25, 50, 75, 100, or 125 bar, and the turbine inlet temperature is either 500, 600, 700, 800, or 900° C. Note that the proposed framework can accommodate a finer discretization scheme for the operating conditions. It is assumed that the pump inlet temperature is equal to the saturation temperature at the given condenser pressure. Using the Aspen Plus v7.1 program and the Peng-Robinson equation of state with the Boston-Mathias alpha function, the electricity used by a pump and delivered by a turbine at any set of valid operating conditions (b, c, t) is calculated. A set of operating conditions is deemed invalid if either (i) the boiler pressure is lower than the condenser pressure or (ii) the specified set of operating conditions causes the working fluid (i.e., water) to condense in the turbine. The amount of energy consumed/delivered per mass of working fluid is determined so that the overall energy delivered by a heat engine can be calculated simply by scaling up the working fluid flow rate. Moreover, since the inlet and outlet conditions of the working fluid are known for both heat exchangers in a heat engine, these may be treated as process streams of unknown flow rate. Splitting of the process streams into a distinct heat exchanger network and a heat engine network is therefore unnecessary.

Although the heat engines allow for the generation of electricity, the HEPN is still able to generate steam at various pressure levels to be used as a feed for specific process units (i.e., gasifiers, autothermal reactor). A large amount of condensate is produced from the process, but this is not enough to satisfy the steam demands from any of the considered CBGTL flowsheets. Process water (25° C., 1 bar) is purchased to make up the difference between the steam requirement and the deaerator condensate. The condensate is output from the sour stripper and is assumed to pass through a deaerator to remove any entrained vapor. If electrolyzers are used to generate hydrogen, the amount of input process water is adjusted to reflect the additional water needed by the electrolyzer units to produce hydrogen. It is assumed that both the condensate and the process water can be directly used in the electrolyzer units without any further adjustment of the stream temperature. Steam production is directly incorporated into the HEPN by first assuming that the condensate will pass through a deaerator and can be pumped to multiple pressure levels where the water is then heated up to the saturation temperature and subsequentially vaporized. If process water is used for steam production, it is first heated up to the deaerator temperature (100° C.) before being mixed with the deaerator outlet.

To ensure a complete integration of the CBGTL process, a comprehensive list of the utility requirements of all process units is compiled (Table 24). This list allows the CBGTL process to directly include the utility requirement of feedstock, product handling, and unit operations when this information is not directly available through Aspen Plus. For instance, operation of the biomass gasifier includes the gasifier, lockhopper, cyclones, and other auxiliary units. Although Aspen Plus blocks can model the material balances within each of these units, no measurement can be made for the electricity required to operate these units or any additional heating or cooling utilities. To estimate what the hot/cold/power utility requirement will be, it is assumed that the requirement will scale linearly with a given process stream flow rate. For instance, if the electricity requirement for gasification (including all auxiliary units) was reported as 13.605 MW for a flow rate of 1 tonne/s, it is assumed that the electricity requirement for any biomass flow rate is calculated by multiplying the flow rate by 13.605 MJ/tonne. Utilities can be calculated in a similar fashion for all units in Table 24. Note that these utilities needed for the CBGTL process are distinct from the utilities needed to develop the HEPN.

Table 24 breaks down the utility requirement into (i) cooling water, (ii) electricity, (iii) plant fuel, (iv) steam required, and (v) steam produced. Prior to the generation of the HEPN, the process electricity requirement is calculated for the recycle compressors/pumps in the Aspen Plus simulation and the units in Table 24. The process cooling water requirement is also calculated using Table 24. These two quantities represent additional utility requirements that must be added as constants to the cost function in the objective in the minimum hot/cold/power utility model and have no effect on the operating conditions of the heat engines that provide the minimum hot/cold/power utility cost. The plant fuel requirement must be taken into account within the CBGTL process to maintain a near-100% conversion of the feedstock carbon. Burning fuel to provide heat will release CO₂, which must react with H₂ in the reverse water-gas-shift (RGS) reactor. Therefore, a fuel combuster is included in the CBGTL simulation, where the flow rate of the feed is adjusted to maintain the exact fuel requirement needed for the rest of the process. The plant fuel temperature was assumed to be 1300° C.

Although the process electricity, cooling water, and plant fuel are directly calculated prior to the development of the HEPN, the steam heating requirements will be fully integrated within the HEPN. To begin, the steam flow rate requirement is changed into a heating requirement by calculating the heat released when steam under the given conditions in Table 24 is cooled to a saturated liquid at the same pressure. This now represents a quantity of heat that is needed at a temperature at least as high as the saturation temperature. Thus, the steam utility requirements of all the units in Table 24 can be thought of as point sinks (requires steam) or point sources (produces steam) of heat at a given temperature.

TABLE 24
Utility Requirements for the Process Flowsheet

| | | | | | | | Stem (Mlb/h) | | | | | |
|---|---|---|---|---|---|---|---|---|---|---|---|---|
| | | | | elec- | fuel | cooling | 600 | 360 | 360 | 150 | 50 | |
| unit | | | | tricity | (MM | water | psig, | psig, | psig, | psig, | psig, | |
| name | unit description | base rate | units | (kW) | BTU/H) | (GPM)^{b} | 650° F. | 600° F. | sat. | sat. | sat. | ref |
| | biomass receiving/ | 1000 | kg/s as received | 10000 | 0 | 0 | 0 | 0 | 0 | 0 | 0 | 11 |
| | storage | | biomass | | | | | | | | | |
| P101 | biomass storage/ | 1000 | Mlb/h bone dry | 13605 | 544 | 0 | 0 | 0 | 0 | 0 | 0 | 16 |
| | drying | | biomass | | | | | | | | | |
| P102 | biomass gasification | 1000 | Mlb/h bone dry | 41905 | 0 | 0 | 0 | 0 | 0 | 0 | 0 | 16 |
| | | | biomass | | | | | | | | | |
| | coal receiving/storage | 1547.705 | Mlb/h bone dry coal | 1703 | 0 | 0 | 0 | 0 | 0 | 0 | 0 | 17.19 |
| P105 | coal drying/grinding | 1547.705 | Mlb/h bone dry coal | 23905 | 210 | 0 | 0 | 0 | 0 | 0 | 0 | 17.19 |
| P106 | coal gasification | 1547.705 | Mlb/h bone dry coal | 44000 | 0 | 0 | 0 | 0 | 0 | 0 | 0 | 17.19 |
| P201 | reverse water-gas shift | 10 | MM SCF/h syngas | 24665 | 0 | 0 | 0 | 0 | 0 | 0 | 0 | 16 |
| P202 | COS/HCN hydrolysis | 55.255 | MM SCF/h syngas | 2201 | 0 | 0 | 0 | 0 | 0 | 0 | 0 | 17.19 |
| P203 | Two-Stage Rectisol | 10000 | kmol/h (CO₂ + H₂S) | 5278 | 0 | 0 | 0 | 0 | 0 | 0 | 153.7 | 11 |
| | (no ref.) | | | | | | | | | | | |
| P203 | Two-Stage Rectisol | 100 | kW_{T} for cooling | 300 | 0 | 0 | 0 | 0 | 0 | 0 | 0 | 11 |
| | (refrig.) | | input to 12° C. | | | | | | | | | |
| P301 | high temperature FT | 64.059 | MM SCF/h syngas | 6958 | 150 | 0 | 33 | 0 | 0 | 0 | 0 | 18.19 |
| P302 | low temperature FT | 64.059 | MM SCF/h syngas | 6958 | 150 | 0 | 33 | 0 | 0 | 0 | 0 | 18.19 |
| P401 | hydrocarbon recovery | 10000 | Mlb/h feed | 8780 | 727.67 | 601.4 | 0 | 0 | 0 | 0 | 576.04 | 16 |
| P402 | wax hydrocracker | 284.845 | Mlb/h feed | 1984 | 88.8 | 219 | 66 | 204 | 0 | 0 | −180 | 18.19 |
| P403 | distillate hydrotreater | 91.454 | Mlb/h feed | 1067 | 11.74 | 187 | 0 | 0 | 0 | 0 | −3 | 18.19 |
| P404 | kerosene hydrotreater | 91.454 | Mlb/h feed | 1067 | 11.74 | 187 | 0 | 0 | 0 | 0 | −3 | 18.19 |
| P405 | naphtha hydrotreater | 99.932 | Mlb/h feed | 740 | 57.18 | 2856 | 0 | 0 | 0 | 0 | 0 | 18.19 |
| P406 | naphtha reformer | 124.88 | Mlb/h feed | 2933 | 108.5 | 747 | −22 | 0 | 0 | 0 | 0 | 18.19 |
| P407 | C₅/C₆ isomerizer | 54.296 | Mlb/h feed | 92 | 3.29 | 51 | 6 | 0 | 0 | 0 | −1 | 18.19 |
| P409 | C₄ isomerizer | 49.415 | Mlb/h feed | 680 | 1.59 | 68 | 7 | 0 | 0 | 0 | 71 | 18.19 |
| P410 | C₃/C₄/C₅ alkylation | 101.308 | Mlb/h feed | 6596 | 0 | 1216 | 17 | 0 | 0 | 0 | 44 | 18.19 |
| | unit | | | | | | | | | | | |
| P411 | saturated gas plant | 34.308 | Mlb/h feed | 93 | 10.67 | 1204 | 9 | 0 | 0 | 0 | −2 | 18.19 |
| P414 | Single-Stage Rectisol | 10000 | kmol/h (CO₂ + H₂S) | 5278 | 0 | 0 | 0 | 0 | 0 | 0 | 153.7 | 11 |
| | (no ref.) | | | | | | | | | | | |
| P414 | Single-Stage Rectisol | 100 | kW_{T} for cooling | 300 | 0 | 0 | 0 | 0 | 0 | 0 | 0 | 11 |
| | (refrig.) | | input to 12° C. | | | | | | | | | |
| P501 | air separation unit | 4560 | TPD,^{c} 95 mol % | 1000 | 0 | 0 | 0 | 0 | 14.8 | 0 | 14.7 | 12 |
| | | | oxygen output | | | | | | | | | |
| P602 | Claus plant | 147 | TPD fed to P602 | 200 | 0 | 0 | 0 | 0 | 0 | 0 | 0 | 12 |
| | offsite | 529.561 | Mlb/h gasoline, | 14889 | 0 | 0 | 0 | 0 | 0 | 0 | 0 | 19 |
| | | | diesel, kerosene | | | | | | | | | |

^{a}Positive and negative steam values correspond to consumption and production, respectively.
^{b}GPM = gallons per minute.
^{c}TPD = metric tons per day.

### Example 2.2

### Mathematical Model for Hot/Cold/Power Utility Minimization

This example describes the mathematical model used to find the minimum hot/cold/power utility cost. A restricted utility model is used to prevent heat flow between streams that are either infeasible or are undesirable. These restrictions are imposed mainly for the point sources of heat that correspond to process units that require a cooling jacket and include the coal gasifier, the Fischer-Tropsch (FT) units, the Claus furnace, and the Claus sulfur separators. As all of these units have a negative heat duty, they generally will form steam within the plant. By electing to incorporate these units in the HEPN, care must be taken to prevent them from transferring heat to a process stream. To mitigate a potential safety risk in the plant, only the heat engines will be allowed to absorb heat from these units.

Indices. The indices for this model will be equivalent to those used for the other stages of the decomposition. They are defined here and referenced in subsequent sections.

i: Hot stream/heat source index
j: Cold stream/heat sink index
k: Temperature interval index
b: Boiler pressure index
c: Condenser pressure index
s: Subnetwork index
t: Turbine inlet temperature index

Parameters. The following mass flow rate parameters are directly extracted from the Aspen Plus simulation report.

Fᵢ^{HP}: Mass flow rate of hot process stream i
Fⱼ^{CP}: Mass flow rate of cold process stream j
F_{Dea}: Deaerator water outlet available for steam generation
Fᵢ^{Proc}: Amount of generated steam utility i that is needed for the process units

The thermal parameters are calculated using Aspen Plus heating curves. The point source heat duties are nonzero only in the specific temperature interval where heat is released/absorbed. The heat capacities are temperature-interval-dependent and are calculated as the average value of the heat capacity at the bounds of the temperature interval. The relevant stream information for the three hybrid flowsheets (i.e., H-R-A, H-EA, and H-R-T) are included. This information includes (i) the process stream flow rates, (ii) the process stream heating curves, and (iii) the heat duty given off by the point sources.

C_{i,k}^{HP}: Specific heat capacity for hot process stream i in temperature interval k
C_{j,k}^{CP}: Specific heat capacity for cold process stream j in temperature interval k
C_{j,k}^{CU}: Specific heat capacity for cold utility stream j in temperature interval k
C_{i,k}^{HG}: Specific heat capacity for hot generated utility stream i in temperature interval k
C_{(b,c,t),k}^{HE}: Specific heat capacity for heat engine (b, c, t) hot fluid in temperature interval k
C_{(b,c,t),k}^{CE}: Specific heat capacity for heat engine (b, c, t) cold fluid in temperature interval k
Q_{i,k}^{HPt}: Heat released by heat source i in temperature interval k
Q_{j,k}^{CPt}: Heat absorbed by heat sink j in temperature interval k
The remaining parameters are listed below. The possible working conditions of the heat engine correspond to a given amount of produced electricity in the turbine and consumed electricity in the pump. The parameters W_{(b,c,t)}^{Tur}, W_{(b,c,t)}^{Pum}, and T_{(b,c,t)}^{Min} are calculated using Aspen Plus assuming (a) a 95% mechanical efficiency of the turbine and pump drivers, (b) a 75% isentropic efficiency of the turbine, (c) and a pump efficiency calculated using Aspen Plus default methods.
P_{b}^{B}: Working pressure of boiler b
P_{c}^{C}: Working pressure of condenser c
Tₜ: Turbine inlet temperature
W_{(b,c,t)}^{Tur}: Specific energy generated by heat engine (b, c, t) turbine
W_{(b,c,t)}^{Pum}: Specific energy used by heat engine (b, c, t) pump
T_{(b,c,t)}^{Min}: Minimum turbine inlet temperature required to maintain vapor phase within the turbine
EnMax: The maximum number of heat engines allowed in the HEPN

The final set of parameters is associated with the temperature intervals of the process flowsheet. The temperature intervals are derived by first determining the inlet temperature for each process stream, utility stream, and heat engine stream, as well as the temperature for all heat sources. All values for the hot streams are then decreased by the minimum temperature approach (ΔTₘᵢₙ) 10° C.) and a set of all unique temperature values is ordered by decreasing temperature value. A temperature interval is defined as the region of temperatures between any adjacent values in the descending list. If the stream outlet temperature is not within the temperature interval, then the value of ΔT for that particular stream in that interval is equal to the full ΔT of the interval. If the outlet temperature is contained within the interval, then the stream ΔT value is equal to the difference between the outlet temperature and the interval bound that passes through the stream temperature range. Note that this criterion does not have to be used with the inlet stream temperatures, because they were used to construct the bounds of the temperature intervals.

ΔT_{i,k}^{H}: Temperature difference of hot stream i in interval k
ΔT_{j,k}^{C}: Temperature difference of cold stream j in interval k
ΔT_{(b,c,t),k}^{HE}: Temperature difference of heat engine (b, c, t) hot stream in interval k
ΔT_{(b,c,t),k}^{CE}: Temperature difference of heat engine (b, c, t) cold stream in interval k
ΔTₘᵢₙ: Minimum temperature interval approach temperature

Sets. The sets used in this model correspond to the temperature intervals (TI), as well as the process streams (HP and CP), utilities (HG and CU), or point sources (HPt and CPt).

TI: {k|k is a HEPN temperature interval}
HP: {i|i is a hot process stream}
HPt: {i|i is a hot point source}
HG: {i|i is a generated steam utility stream}
CP: {j|j is a cold process stream}
CPt: {j|j is a cold point source}
CU: {j|j is a cold utility}
Eng: {(b, c, t)|(b, c, t) is a feasible heat engine}

Note that there are several (b, c, t) heat engine triplets that correspond to discrete combinations of impractical operating conditions within the turbine. Thus, not all (b, c, t) combinations will be included in the model. To restrict the turbines to feasible operating conditions, the following criteria are imposed on the operating conditions of a turbine:

P_{b}^{B}>P_{c}^{C}

Tₜ≧T_{b,c}^{min}

where T_{b,c}^{min} is the minimum temperature needed to maintain a vapor phase in the turbine during expansion from P_{b}^{B} to P_{c}^{C}. Similarly, a feasible pump is defined by imposing P_{b}^{B}>P_{c}^{C}. A heat engine is considered feasible if the pump conditions are feasible and the vapor phase is maintained within the turbine. Although the optimizer could prevent an infeasible operating condition based on the objective function (i.e., zero work for the turbine or infinite work for the pumps), to reduce the computational complexity, these infeasible operating conditions are removed prior to construction of the model.

Variables. We use continuous variables to represent heat transfer Q, residual heat flow R, and fluid flow rate F of the working fluid in the heat engine or of a utility. We define the unrestricted hot and cold streams to represent all hot and cold streams that do not have any restrictions on the matches. For example, in a given temperature interval k, we look at the total heat transferred by the hot streams that do not have match restrictions and define the unrestricted hot stream as the composite of all these streams. The same definition applies for the unrestricted cold stream. Binary variables y are introduced to represent the logical use of a heat engine in the HEPN. That is, the variable y_{(b,c,t)}^{En} will be equal to 1 if the engine is present in the HEPN and will be 0 otherwise. The formal variable list is defined below.

R_{i,k}^{H}: Residual heat flow of restricted stream i from temperature interval k
Rₖ^{h}: Total residual heat flow of all unrestricted hot streams from temperature interval k
Q_{i,k}^{H}: Heat delivered by restricted hot stream i in interval k
Q_{j,k}^{C}: Heat absorbed by restricted cold stream j in interval k
Qₖ^{h}: Total heat delivered by all unrestricted hot streams in interval k
Qₖ^{c}: Total heat absorbed by all unrestricted cold streams in interval k
Q_{i,j,k}^{HC}: Heat transferred from restricted hot stream i to restricted cold stream j in interval k
Q_{i,k}^{Hc}: Heat transferred from restricted hot stream i to unrestricted cold stream in interval k
Q_{j,k}^{nC}: Heat transferred from unrestricted hot stream to restricted cold stream j in interval k
Qₖ^{hc}: Heat transferred from unrestricted hot stream to unrestricted cold stream in interval k
F_{(b,c,t)}^{En}: Flow rate of the working fluid in heat engine (b, c, t)
Fᵢ^{HG}: Flow rate of generated hot utility i
Fⱼ^{CU}: Flow rate of cold utility j
F_{El}: Flow rate of electricity generated

Constraints. The unrestricted heat flow is initially defined by lumping all streams that are allowed to transfer heat to any other part of the process. Specifically, this refers to the heat engine streams, as well as the consumed and the generated utility streams, since there are no physical or practical limitations on heat transfer to or from these streams. The unrestricted heat flow is defined for hot streams in eq 130 and for cold streams in eq 131. In each equation, the heat flow for a process stream is defined as the product of the mass flow rate (F), the heat capacity (C), and the temperature change (ΔT). The mass flow rate for the heat engines F_{(b,c,t)}^{En}, the cold utility (i.e., cooling water) Fⱼ^{CU}, and the hot generated utility (i.e., generated steam) Fᵢ^{HG} are variables that will be selected by the mathematical model. All heat capacities and temperature changes are output of the Aspen Plus software and are known parameters. The total heat delivered by each of these streams in a temperature interval k is summed to generate a hot Qₖ^{h} and cold Qₖ^{c} composite stream.

$$\begin{matrix} {\sum }_{( b , c , t ) ∈ Eng} {F}_{( b , c , t )}^{En} {C}_{( b , c , t ) , k}^{HE} Δ {T}_{( b , c , t ) , k}^{HE} = {Q}_{k}^{h} ∀ k ∈ TI & ( 130 ) \\ {\sum }_{( b , c , t ) ∈ Eng} {F}_{( b , c , t )}^{En} {C}_{( b , c , t ) , k}^{CE} Δ {T}_{( b , c , t ) , k}^{CE} + {\sum }_{i ∈ HG} {F}_{i}^{HG} {C}_{i , k}^{HG} Δ {T}_{i , k}^{H} + {\sum }_{j ∈ CU} {F}_{j}^{CU} {C}_{j , k}^{CU} Δ {T}_{j , k}^{C} = {Q}_{k}^{c} \\ ∀ k ∈ TI & ( 131 )\end{matrix}$$

The energy balances for the remaining streams are given by eqs 132-137. Note that the energy balances for the point sources (eqs 135 and 137) do not include heat terms from the other point sources or the process streams. Also, the energy balances for the process streams (eqs 134 and 136) do not include heat terms for the point sources. Thus, the energy balances only contain desirable heat matches for the process.

$$\begin{matrix}{R}_{k}^{h} - {R}_{k - 1}^{h} + {\sum }_{j ∈ CP ⋃ CPt} {Q}_{j , k}^{hC} + {Q}_{k}^{hc} = {Q}_{k}^{h} ∀ k ∈ TI & ( 132 ) \\ {\sum }_{i ∈ HP ⋃ HPt} {Q}_{i , k}^{Hc} + {Q}_{k}^{hc} = {Q}_{k}^{c} ∀ k ∈ TI & ( 133 ) \\ {R}_{i , k}^{H} - {R}_{i , k - 1}^{H} + {\sum }_{j ∈ CP} {Q}_{i , j , k}^{HC} + {Q}_{i , k}^{Hc} = {F}_{i}^{HP} {C}_{i , k}^{HP} Δ {T}_{i , k}^{H} \\ ∀ i ∈ HP , k ∈ TI & ( 134 ) \\ {R}_{i , k}^{H} - {R}_{i , k - 1}^{H} + {Q}_{i , k}^{Hc} = {Q}_{i , k}^{HPt} ∀ i ∈ HPt , k ∈ TI & ( 135 ) \\ {\sum }_{i ∈ HP} {Q}_{i , j , k}^{HC} + {Q}_{j , k}^{hC} = {F}_{j}^{CP} {C}_{j , k}^{CP} Δ {T}_{j , k}^{C} ∀ j ∈ CP , k ∈ TI & ( 136 ) \\ {Q}_{j , k}^{hC} = {Q}_{j , k}^{CPt} ∀ j ∈ CPt , k ∈ TI & ( 137 )\end{matrix}$$

Constraints to govern operation of the heat engines must ensure the proper output of electricity for the working fluid flow rate. The electricity generated by a heat engine can be calculated by subtracting the pump requirement from the turbine output (eq 138). To prevent the excessive use of heat engines, we must set the maximum number of heat engines (eq 139) and ensure that the working fluid flow rate is nonzero if and only if the engine is operating in the HEPN (eq 140).

$$\begin{matrix}{\sum }_{( b , c , t ) ∈ Eng} ( {w}_{( b , c , t )}^{Tur} - {w}_{( b , c , t )}^{En} ) {F}_{( b , c , t )}^{En} = {F}_{EI} & ( 138 ) \\ {\sum }_{( b , c , t ) ∈ Eng} {y}_{( b , c , t )}^{En} \leq EnMax & ( 139 ) \\ {F}_{( b , c , t )}^{Up} {y}_{( b , c , t )}^{En} \geq {F}_{( b , c , t )}^{En} ∀ ( b , c , t ) ∈ Eng & ( 140 )\end{matrix}$$

The value of EnMax is set to 3 and that of F_{(b,c,t)}^{Up} to an upper bound of 103 kg/s. The imposed upper bound does not restrict the feasible set of operating conditions for the heat engines for the seven CBGTL processes. A set of constraints are imposed to ensure that the water used by the system is balanced. We assume that the cooling water will be part of a system that is regenerated using a cooling tower and is thus isolated from the process water. The specification of zero hot utilities leaves two balances that must be imposed on the water available for steam generation (eq 141) and the steam needed for the process units (eq 142). Thus, it is ensured that all of the deaerator outlet is transferred to steam either for use within the process or for resale.

$$\begin{matrix}{\sum }_{i ∈ HG} {F}_{i}^{HG} = {F}_{Dea} & ( 141 ) \\ {F}_{i}^{HG} \geq {F}_{i}^{Proc} ∀ i ∈ HG & ( 142 )\end{matrix}$$

We seek to minimize the total cost of the system, as defined by eq 143:

$$min {\sum }_{j ∈ CU} {Cost}_{j}^{CU} {F}_{j}^{CU} - {\sum }_{i ∈ HG} {Cost}_{i}^{HG} {F}_{i}^{HG} - {Cost}_{EI} {F}_{EI} \qquad ( 143 )$$

Thus, the complete model is given as

$$min {\sum }_{j ∈ CU} {Cost}_{j}^{CU} {F}_{j}^{CU} - {\sum }_{i ∈ HG} {Cost}_{i}^{HG} {F}_{i}^{HG} - {Cost}_{EI} {F}_{EI}$$

$$subject to$$

$${\sum }_{( b , c , t ) ∈ Eng} {F}_{( b , c , t )}^{En} {C}_{( b , c , t ) , k}^{HE} Δ {T}_{( b , c , t ) , k}^{HE} = {Q}_{k}^{h} ∀ k ∈ TI$$

$${\sum }_{( b , c , t ) ∈ Eng} {F}_{( b , c , t )}^{En} {C}_{( b , c , t ) , k}^{CE} Δ {T}_{( b , c , t ) , k}^{CE} + {\sum }_{i ∈ HG} {F}_{i}^{HG} {C}_{i , k}^{HG} Δ {T}_{i , k}^{H} + {\sum }_{j ∈ CU} {F}_{j}^{CU} {C}_{j , k}^{CU} Δ {T}_{j , k}^{C} = {Q}_{k}^{c}$$

$$∀ k ∈ TI$$

$${R}_{k}^{h} - {R}_{k - 1}^{h} + {\sum }_{j ∈ CP ⋃ CPt} {Q}_{j , k}^{hC} + {Q}_{k}^{hc} = {Q}_{k}^{h} ∀ k ∈ TI$$

$${\sum }_{i ∈ HP ⋃ HPt} {Q}_{i , k}^{Hc} + {Q}_{k}^{hc} = {Q}_{k}^{c} ∀ k ∈ TI$$

$${R}_{i , k}^{H} - {R}_{i , k - 1}^{H} + {\sum }_{j ∈ CP} {Q}_{i , j , k}^{HC} + {Q}_{i , k}^{Hc} = {F}_{i}^{HP} {C}_{i , k}^{HP} Δ {T}_{i , k}^{H} = 0$$

$$∀ i ∈ HP , k ∈ TI$$

$${R}_{i , k}^{H} - {R}_{i , k - 1}^{H} + {Q}_{i , k}^{Hc} = {Q}_{i , k}^{HPt} ∀ i ∈ HPt , k ∈ TI$$

$${\sum }_{i ∈ HP} {Q}_{i , j , k}^{HC} + {Q}_{j , k}^{hC} = {F}_{j}^{CP} {C}_{j , k}^{CP} Δ {T}_{j , k}^{C} = 0 ∀ j ∈ CP , k ∈ TI$$

$${Q}_{j , k}^{hC} = {Q}_{j , k}^{CPt} ∀ j ∈ CPt , k ∈ TI$$

$${\sum }_{( b , c , t ) ∈ Eng} ( {w}_{( b , c , t )}^{Tur} - {w}_{( b , c , t )}^{Pum} ) {F}_{( b , c , t )}^{En} = {F}_{EI}$$

$${\sum }_{( b , c , t ) ∈ Eng} {y}_{( b , c , t )}^{En} \leq EnMax$$

$${F}_{( b , c , t )}^{Up} {y}_{( b , c , t )}^{En} \geq {F}_{( b , c , t )}^{En} ∀ ( b , c , t ) ∈ Eng$$

$${\sum }_{i ∈ HG} {F}_{i}^{HG} = {F}_{Dea}$$

$${F}_{i}^{HG} \geq {F}_{i}^{Proc} ∀ i ∈ HG$$

Equations 130-143 represent a mixed-integer linear optimization (MILP) model that can be solved to global optimality using CPLEX13 to obtain (i) the active binary variables y_{(b,c,t)}^{En} that represent the operating conditions of the heat engine, (ii) the values of the working fluid flow rates of the heat engines F_{(b,c,t)}^{En}, (iii) the amount of electricity produced by the heat engines F^{El}, and (iv) the flow rate of the cooling utility Fⱼ^{CU}.

### Example 2.3

### Computational Results

Upon completion of the simulation for a given flowsheet, several key pieces of data are extracted from the simulation results to determine (i) steam demand for the process units, (ii) available condensate, (iii) the electricity requirement of the compressors, and (iv) the initial cooling water and electricity requirement for other process units using the information in Table 24. This information is presented in Table 25. Note that all results are normalized with respect to the total volume of products (in bbl). Since each process simulation had a total of 2000 tonnes/day of combined biomasscoal-natural gas feedstock, normalizing the results with respect to the products allows for a direct comparison of overall utility usage, as well as overall cost.

TABLE 25
Process Utility Requirements for the CBGTL Flowsheets^{a}

| | CW | CN | Steam Demand (kg/bbl)^{b} | | | | | | Elec |
|---|---|---|---|---|---|---|---|---|---|
| process | (kg/bbl) | (kg/bbl) | @ 5 bar | @ 25 bar | @ 35 bar | @ 45bar | @ 75 bar | @ 125 bar | (GJ/bbl) |
| B-R-A | 50.13 | 79.81 | 0 | 0 | 84.02 | 0 | 0 | 0 | 0.773 |
| B-E-A | 49.98 | 79.99 | 0 | 0 | 84.13 | 0 | 0 | 0 | 4.432 |
| C-R-A | 53.86 | 88.24 | 0 | 0 | 92.01 | 0 | 0 | 0 | 0.831 |
| C-E-A | 53.14 | 88.36 | 0 | 0 | 92.21 | 0 | 0 | 0 | 4.780 |
| H-R-A | 52.08 | 85.38 | 0 | 0 | 89.38 | 0 | 0 | 0 | 0.802 |
| H-E-A | 51.41 | 85.57 | 0 | 0 | 89.59 | 0 | 0 | 0 | 4.610 |
| H-R-T | 41.26 | 47.33 | 0 | 0 | 53.51 | 0 | 0 | 0 | 0.786 |

^{a}Each flowsheet provides (i) the total steam demand for the process units, (ii) the available condensate (CN), and (iii) the initial values for the cooling water (CW) and electricity (Elec).
^{b}All results are normalized with respect to the total volume of products (bbl; barrel).

The total amount of required cooling water, available condensate, and process units steam requirement is similar for all cases except H-R-T. The decreased values for the H-R-T flowsheet result from a loss of CO₂ in the gas turbine section, which subsequentially reduces the recycle vapor-phase flow rate throughout the process. In addition, since the autothermal reactor does not interact with the recycle vapor phase, there is a decrease both in the amount of pure oxygen and the amount of steam needed for the process. Next, the significant difference in electricity requirement for the electrolyzer cases (E) is highlighted, as opposed to the air separation unit (ASU; R) cases. Although the lack of the air and pure oxygen compressors reduces the electricity load, this is negligible to the electricity requirement of the electrolyzers. These units are assumed to operate at 75% of the thermodynamic efficiency14 and, therefore, require 188.96 MJ/kg H₂ produced.

The total utility requirement after completion of the minimum utility model is presented in Table 26. For each of the process flowsheets, the necessary cooling water flow for the HEPN is much larger than the additional requirement of the process units. This value does not represent the amount of cooling water that must be input to the process. Rather, this number is representative of the flow rate of cooling water through the process. The amount of process water that must be purchased is equal to the difference between the steam requirement and the condensate flow rate in Table 25. The amount of cooling water is generally higher for the electrolyzer cases, compared to the ASU cases. This is likely due to the low pressure steam requirement of the ASU. For the electrolyzer cases, some excess low temperature heat is exiting the process through cooling water as opposed to steam. In addition, the cooling water requirement of the gas turbine system is 1.5 times higher than the other cases. A large amount of waste heat is generated from the cooling of the gas turbine outlet, some of which cannot be recovered and exits the process in the cooling water.

TABLE 26
Results of the Minimum Hot/Cold/Power Utility Model^{a}

| | CW | PW | Steam Demand (kg/bbl)^{b} | | | | | | Elec. | Util. |
|---|---|---|---|---|---|---|---|---|---|---|
| process | (kg/bbl) | (kg/bbl) | @ 5 bar | @ 25 bar | @ 35 bar | @ 45bar | @ 75 bar | @ 125 bar | (GJ/bbl) | ($/bbl) |
| B-R-A | 18931 | 4.21 | 0 | 0 | −84.02 | 0 | 0 | 0 | 0.135 | 2.856 |
| B-E-A | 21986 | 4.14 | 0 | 0 | −84.13 | 0 | 0 | 0 | 3.912 | 65.92 |
| C-R-A | 15998 | 3.87 | 0 | 0 | −92.01 | 0 | 0 | 0 | 0.282 | 5.213 |
| C-E-A | 16190 | 3.85 | 0 | 0 | −92.21 | 0 | 0 | 0 | 4.101 | 68.88 |
| H-R-A | 18280 | 4.00 | 0 | 0 | −89.38 | 0 | 0 | 0 | 0.209 | 4.069 |
| H-E-A | 17474 | 4.02 | 0 | 0 | −89.59 | 0 | 0 | 0 | 4.000 | 67.24 |
| H-R-T | 30464 | 6.18 | 0 | 0 | −53.51 | 0 | 0 | 0 | −0.107 | −0.809 |
| Cost | $31.79/ | $953.8/ | | | | | | | $16.67/ | |
| | 10⁶ kg | 10⁶ kg | | | | | | | GJ | |

^{a}The electricity (Elec.) is equal to the sum of the process electricity plus that produced by the heat engines. The process water (PW) is equal to the difference between the steam required by the process units (i.e., gasifiers and ATR) and the condensate output from the deaerator. The cooling water (CW) is equal to the sum of the process unit requirement and the HEPN requirement. The produced steam is given and represents the requirement for the gasifiers and auto-thermal reactor. Since steam is not sold as a byproduct, this is not included in the total utility cost.
^{b}All results are normalized with respect to the total volume of products (bbl: barrel).

The electricity requirement in Table 26 represents the sum from the process, as well as that recovered from the HEPN. The only process that is able to provide a negative utility cost (from sale of the electricity) is the gas turbine system. This was anticipated since this flowsheet will have smaller recycle compression costs due to removal of the CO₂. However, the benefit is reduced somewhat due to the loss of carbon from the system, because not as much product will be made. The total electricity requirement of the remaining flowsheets is the smallest for pure biomass, slightly larger for the hybrid system, and largest for the pure coal processes. Furthermore, for any given feedstock, the electricity requirement for the ASU cases is more than 1 order of magnitude lower than that for the electrolyzer cases and is a direct consequence of the high electrolyzer requirement (Table 25). The overall cost of each system is strongly dependent on the amount of electricity needed; therefore, it is important to reduce the electricity usage of the electrolyzers as much as possible. Even when operating at 100% thermodynamic efficiency, the units will still require 141.72 MJ/kg H₂ produced, so the key will be reducing the hydrogen requirement via a formulation of a rigorous process synthesis problem.

For these results presented above, several possible heat engines were postulated, including four condenser pressures (P_{c}^{C}∈{1 bar, 5 bar, 15 bar, 40 bar}), five boiler pressures (Pb B∈{25 bar, 50 bar, 75 bar, 100 bar, 125 bar}), (P_{c}^{C}∈{1 bar, 5 bar, 15 bar, 40 bar}), five boiler pressures (P_{b}^{B}∈{25 bar, 50 bar, 75 bar, 100 bar, 125 bar}), and five turbine inlet temperatures (Tt∈{500° C., 600° C., 700° C., 800° C., 900° C.}). When placing an upper bound on the total amount of heat engines (i.e., the number of steam turbines) equal to three, the resulting operating conditions are given in Table 27. Note that each process selected three heat engines, although the selection of operating conditions varies even between the process flowsheets with the same feed. This is a result of the absence/presence of the ASU and the necessary steam requirement. We note that in no case is the 125 bar boiler pressure selected. This is possibly due to the saturation temperature of the boiler (326.9° C.), which is above the operating temperature of both FT units (240 and 320° C.). These units will provide a significant amount of waste heat that will need to be recovered by the heat engines to provide the maximum amount of electricity. In addition, note that the triplet (P_{c}^{C}, P_{b}^{B}, Tₜ)=(25, 1, 900) was selected for six of the seven flowsheets, and this selection had the highest working fluid flow rate for each of the flowsheets. The maximum amount of work that is produced for a given boiler pressure is given by the maximum operating turbine inlet temperature and the minimum available condenser pressure. Furthermore, the boiler pressure of 25 bar has a saturation temperature of 223.9° C., which is lower than both operating temperatures (within the minimum temperature approach) of the FT units. The combination of both pieces of information is likely the reason for the common selection of this engine.

TABLE 27
Heat Engine
Configuration for the Optimal Hot/Cold/Power Utility Cost

| | Conditions | | | | | |
|---|---|---|---|---|---|---|
| | (P_{b}^{B} (bar), P_{c}^{C} (bar), Tₜ (° C.)) | | | Working Fluid Flow (kg/s) | | |
| process | En. 1 | En. 2 | En. 3 | En. 1 | En. 2 | En. 3 |
| C-R-A | (25, 1, | (50, 1, | (25, 1, | 30.43 | 5.12 | 8.03 |
| | 900) | 800) | 800) | | | |
| C-E-A | (25, 1, | (50, 1, | (75, 1, | 28.91 | 5.82 | 15.01 |
| | 900) | 700) | 900) | | | |
| B-R-A | (25, 1, | (50, 15, | (25, 1, | 40.12 | 8.23 | 21.12 |
| | 900) | 900) | 800) | | | |
| B-E-A | (25, 1, | (50, 5, | (100, 1, | 34.36 | 15.23 | 6.99 |
| | 900) | 800) | 700) | | | |
| H-R-A | (25, 1, | (75, 40, | (100, 15, | 72.91 | 11.51 | 9.05 |
| | 900) | 900) | 900) | | | |
| H-E-A | (25, 1, | (25, 15, | (75, 40, | 76.04 | 15.21 | 19.34 |
| | 900) | 500) | 900) | | | |
| H-R-T | (25, 1, | (75, 1, | (100, 15, | 61.76 | 57.68 | 25.01 |
| | 600) | 900) | 600) | | | |

### Example 2.4

### Minimum Number of Heat Exchanger Matches

The minimum hot/cold/power utility model has provided us with (i) the required amount of cooling water, (ii) the different levels of steam produced using the deaerator water, (iii) the amount of additional process water needed to produce process steam, (iv) the operating conditions and working fluid flow rate of the heat engines, and (v) the location of the pinch points denoting the distinct subnetworks. Given this information, the minimum heat exchanger matches are calculated that are necessary to meet specifications (i), (ii), (iii), and (iv). Note that the turbines and pumps used in the heat engines, as well as their corresponding working flow rates, are already defined based on the results of the minimum hot/cold/power utility model. Thus, the cost of these units is now fixed, and will not have to be taken into account in a minimization of the total annualized cost of the HEPN.

The formulation of a general minimum heat exchanger matches model results in multiple solutions yielding the same minimum value. A nonlinear minimum annualized cost model will have to be developed for each solution, so it is important to distinguish among these solutions at this stage of the decomposition. Specifically, the focus is on the methods of vertical heat transfer and weighted matches. The vertical heat transfer model adds a penalty to the objective function that is incremented when “criss-cross” heat transfer is used. This method relies on the assumption that maximization of the vertical heat transfer will lead to the minimum heat transfer area for a given number of heat exchanger matches. A weighted matches model assigns a priority to each possible stream match based on proximity within the process flowsheet. The priority does not have a connection with the possible heat transfer area associated with a stream match; it is designed to be an indication of the auxiliary costs associated with a match. The weight for a match is assigned based on the match priority, and the model objective is the minimization of the sum of the weight of all matches.

The use of either one of the above models results in a reduction in the number of solutions, and we can further distinguish among these solutions by constructing a new objective function that is a linear combination of the objectives for each model. A multiplicative coefficient, γ, is placed in front of the weighted matches objective function to emphasize the relative importance compared to the vertical heat transfer objective function.

### Example 2.5

### Mathematical Model for Heat Exchanger Matches Minimization

The minimum utility model has selected a subset of heat engines that provides the necessary electricity. The sets HOT and COLD are defined as follows:

HOT={i|Hot stream i has a positive flow rate} (144)

COLD={j|Cold stream j has a positive flow rate} (145)

This reassignment serves to eliminate all of the heat engine streams that were not activated in the minimum utility model. The set of potential matches between process streams, MATCHES, is defined based on the restrictions imposed in the minimum utility model (eq 146). Specifically, we restrict a match between a hot process stream and a cold point source (HP×CPt), a cold process stream and a hot point source (HPt×CP), and a hot point source with a cold point source (HPt×CPt).

MATCHES={(i,j)|i∈HOT,j∈COLD,(i,j)∉HPt×CPt∪HP×CPt∪HPt×CP} (146)

The HEPN is first discretized into subnetworks (s∈SUB) based on the temperature intervals (eq 147) for which the residual heat flow is zero (Rₖ=0). This significantly reduces the computational complexity needed to calculate the total heat exchanger matches, because it is assumed that there will be no heat flow between subnetworks. That is, the strict pinch case will be employed for this model using a minimum temperature approach of 10° C.

SUB={s|s is a subnetwork the HEPN}

TIₛ={k∈TI|k′≦k≦k″,k′<k″,R_{k′}=R_{k″}=0,R_{k″}>0∀k′<k′″<k″} (147)

For each subnetwork, the superset of all possible intervals for which a hot stream or cold stream may transfer heat is defined using eqs 148 and 149, respectively:

HOTₛ={(i,k)|i∈HOT,k∈TIₛ,∃k′∈TIₛ,k′≦k,Q_{i,k′}^{H}>0} (148)

COLDₛ={(j,k)|j∈COLD,k∈TIₛ,Q_{j,k}^{C}>0} (149)

The set HOTs includes intervals where Q_{i,k}^{H} can be zero for a given stream i, because of the residual heat flow. The set of all possible matches between streams i and j is then introduced for each subnetwork (eq 150), as well as the set of all possible stream matches for each temperature interval k (eq 151).

MATCHESₛ={(i,j)|(i,j)∈MATCHES,∃k∈TIₛ s.t.(i,k)∈HOTₛ AND(j,k)∈COLDₛ} (150)

MATCHESₛ^{TI}={(i,j,k)|(i,j)∈MATCHESₛ,k∈TIₛ} (151)

Using appropriate binary variables (y_{i,j,s}^{Ex}) for each (i, j)∈MATCHESs, the presence of a heat exchanger can be logically activated or deactivated.

### Example 2.6

### General Heat Transfer

The hot and cold energy balances for the matches are given by eqs 152 and 153, respectively.

$$\begin{matrix}{R}_{i , k}^{H} - {R}_{i , k - 1}^{H} + {\sum }_{( i , j , k ) ∈ {MATCHES}_{s}^{TI}} {Q}_{i , j , k}^{HC} = {Q}_{i , k}^{H} \\ ∀ ( i , k ) ∈ {HOT}_{s} , s ∈ SUB & ( 152 ) \\ {\sum }_{( i , j , k ) ∈ {MATCHES}_{s}^{TI}} {Q}_{i , j , k}^{HC} = {Q}_{i , k}^{C} \\ ∀ ( i , k ) ∈ {COLD}_{s} , s ∈ SUB & ( 153 )\end{matrix}$$

Binary variables y_{i,j,s}^{Ex} are introduced for each element of MATCHESs and are equal to 1 if heat transfer exists between hot stream i and cold stream j in subnetwork s (eq 155) and are equal 0 otherwise. The parameter Q_{i,j}^{max} is defined as the maximum possible heat flow between two streams (eq 154) and is equal to the minimum of the total heat load of each respective stream.

min{Qᵢ^{H},Qⱼ^{C}}=Q_{i,j}^{max} (154)

y_{i,j,s}^{Ex}Q_{i,j}^{max}≧Q_{i,j}^{HC}∀(i,j)∈MATCHESₛ,s∈SUB (155)

### Example 2.7

### Vertical Heat Transfer

To develop the model for vertical heat transfer, we partition the enthalpy into enthalpy intervals (l∈EIₛ) based on the subnetwork s. Q_{i,l}^{H} and Q_{j,l}^{C} are defined to be the heat transferred in enthalpy interval/from hot stream and cold stream j, respectively. The vertical heat transfer between two streams in a subnetwork Q_{i,j,s}^{V} is the sum of minimum possible heat transfer in an enthalpy interval in that subnetwork (eq 156).

$${Q}_{i , j , s}^{V} = {\sum }_{l ∈ {EI}_{s}} min \{ {Q}_{i , j}^{H} , {Q}_{j , l}^{C} \} \\ ∀ ( i , j ) ∈ {MATCHES}_{s} , s ∈ SUB \qquad ( 156 )$$

Slack variables Sl_{i,j,s} are then introduced to measure the amount of “criss-cross” heat transfer between a match (eq 157).

Sl_{i,j,s}≧Q_{i,j,s}−Q_{i,j,s}^{V}∀(i,j)∈MATCHESₛ,s∈SUB (157)

### Example 2.8

### Weighted Matches

To determine the match weights, a priority must first be assigned to each heat exchanger match. This is initially done by considering the process proximity between two units in a match. This proximity may either be analyzed at the unit level or a plant level. If the unit level is observed, then a distance metric should be defined that relates the estimated piping distance necessary to connect the hot/cold pair. The plant distance metric would focus on the discretization of the chemical flowsheet into “plants” where the distance between units in two particular plants is calculated as the number of additional plants between the two original units. The plant distance metric was chosen (Table 28) here, since priority assignment based on individual units may be premature without considering additional costs associated with unit placement in the vicinity of each of the matched process units.

TABLE 28
Distance between Process Plants^{a}

| | Plant | Plant | Plant | Plant | Plant |
|---|---|---|---|---|---|
| | 100 | 200 | 300 | 400 | 600 |
| Plant 100 | 0 | 1 | 2 | 2 | 2 |
| Plant 200 | 1 | 0 | 1 | 1 | 1 |
| Plant 300 | 2 | 1 | 0 | 1 | 2 |
| Plant 400 | 2 | 1 | 1 | 0 | 2 |
| Plant 600 | 2 | 1 | 2 | 2 | 0 |

^{a}The process plant distance is the minimum of all pairwise process path distances for all units in both plants.

Given that each unit exists within a different plant in the process, a process path between process unit PU₁ and another unit PU₂ is defined as any connection that can be made by process streams. The process path distance is defined as the total number of plants (excluding the plant from which PU₁ originated) that have at least one unit along the process path. The minimum process path is then defined as the path with the minimum distance over all possible process paths. The process plant distance is the minimum of all pairwise process path minimum distances for all units in both plants. This process path distance is recorded in Table 28.

Because multiple matches will have the same process plant distance, the stream flow rate is also incorporated in the priority calculation. With the assumption that a larger flow will lead to higher piping costs, the set of all hot and cold streams are then ordered based on increasing flow and assigned a flow priority (Pr_{Fl}) from 1 to the total number of hot and cold streams. The point sources are then ordered from lowest to highest heat transfer and assigned a point source priority (Pr^{Pt}) based on the assumption that a point source with a lower heat will require a smaller vessel jacket.

For each subnetwork s, all possible matches (determined from MATCHESₛ) are then placed in a rank-ordered list by first sorting based on increasing process plant distance, then based on increasing flow priority sum, then based on increasing point source priority sum. For matches with only one point source priority or one flow priority, the sorted value is equal to the value of the single priority. If any two consecutive matches in the rank-ordered list have the same process plant distance, flow priority sum, and point source priority sum, they are sorted based on the increasing total amount of heat transferred between the match. Note that any restricted matches from the minimum utility model are not included in the set of possible matches. Each match is then assigned a priority, Pr_{i,j,s}^{MATCH}, based on the ranking in the final ordered list. The weight for a match can then be calculated as wi, j, s based on eq 158:

$${w}_{i , j , s} = \frac{1}{4} ( {N}_{i , j , s} ) + \frac{{Pr}_{i , j , s}^{MATCH}}{{N}_{i , j , s}} \qquad ( 158 )$$

where N_{i,j,s} is the total number of possible matches and is equal to the cardinality of MATCHESₛ.

Objective. We first attempt to find the minimum number of matches for each subnetwork (MinMatchₛ) without concern for which streams are present in the final solution (eq 159).

$${MinMatch}_{s} = {\sum }_{( i , j ) ∈ {MATCHES}_{s}} {y}_{i , j , s}^{Ex} ∀ s ∈ SUB \qquad ( 159 )$$

The complete model below represents a mixed-integer linear program (x s.

$$min {MinMatch}_{s}$$

$$subject to$$

$${\sum }_{( i , j ) ∈ {MATCHES}_{s}} {y}_{i , j , s}^{Ex} = {MinMatch}_{s}$$

$${R}_{i , k}^{H} - {R}_{i , k - 1}^{H} + {\sum }_{( i , j , k ) ∈ {MATCHES}_{s}^{TI}} {Q}_{i , j , k}^{HC} = {Q}_{i , k}^{H}$$

$$∀ ( i , k ) ∈ {HOT}_{s}$$

$${\sum }_{( i , j , k ) ∈ {MATCHES}_{s}^{TI}} {Q}_{i , j , k}^{HC} = {Q}_{i , k}^{C} ∀ ( i , k ) ∈ {COLD}_{s}$$

$${y}_{i , j , s}^{Ex} {Q}_{i , j}^{max} \geq {Q}_{i , j}^{HC} ∀ ( i , j , k ) ∈ {MATCHES}_{s}^{TI}$$

This model is solved to global optimality using CPLEX¹³ to determine the minimum number of heat exchanger matches (MinMatchₛ) for each subnetwork. To distinguish among the different solutions, an objective function utilizing vertical heat transfer and weighted matches (eq 160) is developed.

$$min {\sum }_{( i , j ) ∈ {MATCHES}_{s}} ( {Sl}_{i , j , s} + γ {w}_{i , j , s} {y}_{i , j , s}^{Ex} ) ∀ s ∈ SUB \qquad ( 160 )$$

To place more importance on the vertical heat transfer criterion, γ is set to a value of 1×10⁻⁶. For each subnetwork, MinMatchₛ is fixed at the value found in the previous model and the resulting MILP is represented below for each subnetwork s.

$$min {\sum }_{( i , j ) ∈ {MATCHES}_{s}} ( {Sl}_{i , j , s} + γ {w}_{i , j , s} {y}_{i , j , s}^{Ex} )$$

$$subject to$$

$${\sum }_{( i , j ) ∈ {MATCHES}_{s}} {y}_{i , j , s}^{Ex} = {MinMatch}_{s}$$

$${R}_{i , k}^{H} - {R}_{i , k - 1}^{H} + {\sum }_{( i , j , k ) ∈ {MATCHES}_{s}^{TI}} {Q}_{i , j , k}^{HC} = {Q}_{i , k}^{H}$$

$$∀ ( i , k ) ∈ {HOT}_{s}$$

$${\sum }_{( i , j , k ) ∈ {MATCHES}_{s}^{TI}} {Q}_{i , j , k}^{HC} = {Q}_{i , k}^{C} ∀ ( i , k ) ∈ {COLD}_{s}$$

$${y}_{i , j , s}^{Ex} {Q}_{i , j}^{max} \geq {Q}_{i , j}^{HC} ∀ ( i , j , k ) ∈ {MATCHES}_{s}^{TI}$$

$${Sl}_{i , j , s} \geq {Q}_{i , j , s} - {Q}_{i , j , s}^{V} ∀ ( i , j ) ∈ {MATCHES}_{s}$$

### Example 2.9

### Computational Results and Illustrative Examples

The results for each subnetwork for all seven process flowsheets are presented in Table 29. It is initially noted that each flowsheet is discretized into three subnetworks, although the number of heat exchanger matches (and, thus, the topology) will be different for each subnetwork. Each of these subnetworks will be analyzed using the minimum annualized cost model that is described in the Examples 2.10-2.17.

As an illustrative example, the results for subnetwork one of each of the three hybrid process flowsheets is presented in Table 30. Although the topology will be different for each case, there are several common streams between each of the subnetworks, including the stream exiting the reverse water-gas shift (RGS) unit (H1), the stream exiting the fuel combuster (H6), the steam input the autothermal reactor (ATR; C6), the inlet natural gas stream (C7), the oxygen input the ATR(C8), and the recycle light gases to the autothermal reactor (C9). Additional streams include the inlet hydrogen to the RGS unit (C1) and the recycle CO₂ to the RGS unit. A common point source of heat was the coal gasifier (H15 for H-R-A; H12 for H-E-A; H17 for H-R-T). The final streams in the subnetworks are the hot (H29 for H-R-A; H27 for H-E-A) and cold (C33-C35 for H-R-A; C31-C33 for H-E-A; C33-C35 for H-R-T) heat engine streams.

TABLE 29
Minimum Matches for the CBGTL Process Alternatives

| | C-R-A | C-E-A | B-R-A | B-E-A | H-R-A | H-E-A | H-R-T |
|---|---|---|---|---|---|---|---|
| Sub- | 11 | 15 | 16 | 14 | 16 | 14 | 12 |
| network | | | | | | | |
| 1 | | | | | | | |
| Sub- | 50 | 41 | 51 | 31 | 68 | 37 | 17 |
| network | | | | | | | |
| 2 | | | | | | | |
| Sub- | 41 | 47 | 41 | 59 | 43 | 55 | 64 |
| network | | | | | | | |
| 3 | | | | | | | |

TABLE 30
Heat Exchanger Matches and Heat Duties for the First Subnetwork of Each Hybrid Flowsheet^{a}

| | | | | | | | |
|---|---|---|---|---|---|---|---|
| match | duty (kW) | match | duty (kW) | match | duty (kW) | match | duty (kW) |

H-R-A: Subnetwork 1

| | | | | | | | |
|---|---|---|---|---|---|---|---|
| H1-C6 | 3417.35 | H1-C7 | 1371.41 | H1-C9 | 1150.46 | H1-C33 | 12417.1 |
| H6-C6 | 2408.45 | H6-C7 | 2074.96 | H6-C8 | 1242.76 | H6-C9 | 1685.59 |
| H6-C33 | 3438.04 | H6-C34 | 544.291 | H6-C35 | 1430.34 | H15-C33 | 33268.3 |
| H15-C34 | 5365.04 | H15-C35 | 4986.69 | H29-C6 | 2630.14 | H29-C34 | 2114.43 |

H-E-A: Subnetwork 1

| | | | | | | | |
|---|---|---|---|---|---|---|---|
| H1-C7 | 3544.52 | H1-C31 | 35323.9 | H1-C32 | 585.28 | H1-C33 | 9431.98 |
| H6-C6 | 3610.38 | H6-C7 | 2074.96 | H6-C8 | 2082.13 | H6-C9 | 1427.67 |
| H6-C31 | 3513.70 | H6-C33 | 2200.53 | H12-C31 | 34532.4 | H12-C33 | 7866.58 |
| H27-C6 | 10379.4 | H27-C9 | 3433.23 | | | | |

H-R-T: Subnetwork 1

| | | | | | | | |
|---|---|---|---|---|---|---|---|
| H1-C1 | 9398.60 | H1-C2 | 7643.79 | H1-C6 | 2355.58 | H1-C33 | 6159.20 |
| H1-C35 | 2726.86 | H6-C1 | 1556.52 | H6-C2 | 1548.74 | H6-C6 | 2074.96 |
| H6-C7 | 824.27 | H6-C8 | 1184.94 | H6-C34 | 4490.94 | H17-C34 | 43620.00 |

^{a}The minimum hot/cold/power utility model provided pinch points of 613.21° C. for the H-R-A flowsheet, 482.54° C. for the H-E-A flowsheet, and 555.58° C. for the H-R-T flowsheet.

### Example 2.10

### Network Topology with Minimum Annualized Cost of Heat Exchange

Upon solution of the minimum matches model, we have the optimal set of stream matches and, thus, aim at determining the heat exchanger topology with the minimum annualized cost. There are two possible types of heat exchanger matches: a match between two process streams and a match between a heat engine stream and a point source. Each point source represents the heat that is required by or absorbed from a particular process unit at a given temperature. Although the evolved heat of reaction for the coal gasifier, the Fischer-Tropsch (FT) unit, and the Claus furnace has been thoroughly modeled in the Aspen Plus simulation, we are only given estimates of heating requirements for other units based on the input flow rate to the unit.

### Example 2.11

### Heat Exchanger Cost Functions

To formulate the annualized cost of the heat exchanger, we consider that each heat exchanger will be a shell-and-tube design. A floating head exchanger will be used for nonevaporating streams, while a kettle reboiler will be used for all evaporating streams. The free on board purchase price (C_{P}) of a heat exchanger is given by eq 161:

C_{P}=F_{P}F_{L}F_{M}C_{B} (161)

where C_{B} is the base purchase cost, F_{P} is a pressure factor, F_{M} is a material factor, and F_{L} is a length factor. The base purchase cost is given by eqs 162 and 163 for the kettle reboiler and the floating heat exchangers, respectively:

$$\begin{matrix}{C}_{B}^{K} = \frac{521.9}{394} exp \{ 11.967 - 0.8709 ln ( A ) + {0.09005 [ ln ( A ) ]}^{2} \} & ( 162 ) \\ {C}_{B}^{P} = \frac{521.9}{394} exp \{ 11.667 - 0.8709 ln ( A ) + {0.09005 [ ln ( A ) ]}^{2} \} & ( 163 )\end{matrix}$$

The base costs are functions of the heat exchanger area (A) and are valid in the range of A=150-12000 ft². Note the scaling factor in the beginning of eqs 164 and 165 is used to convert from the mid-2000 cost index to the August 2009 index, via the CE plant cost index.9 The parameters F_{M} and F_{L} are each assumed to be equal to 1. The pressure factor is determined based on the shellside pressure (P, given in psig), as defined in eq 164.

$${F}_{P} = 0.9803 + 0.018 ( \frac{P}{100} ) + 0.0017 {( \frac{P}{100} )}^{2} \qquad ( 164 )$$

Note that, at this stage of the decomposition, the stream matches are now defined, so we are able to determine the shell-side pressure for a given match. If a stream is vaporizing or condensing, that stream is automatically assigned to the shell side. Otherwise, the lower pressure (or lower temperature) stream is assigned to the shell side.

Given the base purchase cost of a heat exchanger, we may calculate the annualized cost by first finding the annuity factor (AF). Assuming the life of the exchanger to be n years, and assuming an interest rate of i, the value of AF is given by eq 165. The annualized cost (CA) is then given by eq 166, where CM is the annual maintenance cost. The maintenance cost is estimated as a percentage of the purchase cost for a fluid handling process (a_{M}),9 as given by eq 167.

$$\begin{matrix}AF = \frac{1 - \frac{1}{{( 1 + i )}^{n}}}{i} & ( 165 ) \\ {C}_{A} = \frac{{C}_{B}}{AF} + {C}_{M} & ( 166 ) \\ {C}_{M} = {a}_{M} {C}_{B} & ( 167 )\end{matrix}$$

An annualized cost is sought that is defined by a power law, as given by eq 168. An attempt to find the best fit between the true annualized cost (C_{A}) and the estimated annualized cost may be accomplished by adjusting the parameters C₀ and sf in eq 168. Using the Euclidean distance as an objective function, the annualized cost functions for the floating head and kettle reboiler are defined in eqs 169 and 170, respectively. For the CBGTL process, the parameters used are n=30, i=15%, and aₘ=10.3%.

C_{A}^{Est}=C₀A^{sf} (168)

C_{A}^{F}=114.72FₚA^{0.5801} (169)

C_{A}^{K}=154.92FₚA^{0.5801} (170)

### Example 2.12

### Heat Exchanger Overall Heat Transfer Coefficients

The areas used to calculate the annualized cost of a heat exchanger correspond to the outside area of the tubes within the exchanger. Therefore, the overall heat transfer coefficient for the other tube area is defined as (U), as in eq 171.

$$U = \frac{1}{{R}_{f , o} + \frac{1}{{h}_{0}} + \frac{{D}_{o}}{{h}_{i} {D}_{i}} + \frac{{t}_{w} {D}_{o} ln ( \frac{{D}_{o}}{{D}_{i}} )}{{k}_{w} ( {D}_{o} - {D}_{i} )} + \frac{{R}_{f , i} {D}_{o}}{{D}_{i}}} \qquad ( 171 )$$

To estimate the value of U, it is assumed that the tube outside diameter (Dₒ) is equal to 0.75 in., the tube wall thickness (t_{w}) is 0.065 in., and both the inner fouling factor (R_{f,i}) and outer fouling factor (R_{f,o}) are equal to 0.002 h ft²° F./Btu. The material of construction will be carbon steel, which is assumed to have a thermal conductivity (kw) of 20 BTU/h° F. ft. The convective heat transfer coefficients are calculated from the Nusselt number (Nu), as in eq 172:

$$h = \frac{kNu}{L} \qquad ( 172 )$$

where L is the characteristic length and k is the thermal conductivity of the fluid. The characteristic length of the tubeside fluid is given by L=Dₒ−Dᵢ, whereas that of the shellside fluid is given by L=(πDₒ)/2. The thermal conductivity of the fluid is given by the Aspen Plus program as a function of temperature and is averaged for each stream across the temperature interval of interest. The Nusselt number (Nu) is given by eq 173, where the value will be constant for Reynolds numbers (Re) of <3000 and is defined by the Gnielinski correlation for Re>3000.

$$Nu = \{ \begin{matrix}4.36 & ∀ Re < 3000 \\ \frac{( f \text{/} 8 ) ( Re - 1000 ) Pr}{1 + 12.7 {( f \text{/} 8 )}^{0.5} ( {Pr}^{2 / 3} - 1 )} & ∀ Re \geq 3000\end{matrix} \qquad ( 173 )$$

The Reynolds number (Re) is given by eq 45, where Q is the volumetric flow rate, L the characteristic length, v the kinematic viscosity, and A the cross-sectional area. Both Q and v are determined from the Aspen Plus program, and the area is defined by the expression A=¼πDᵢ² for tube flow and A=DLₜ for shell flow where Lₜ is equal to the tube length (estimated to be 20 ft). The Prandlt number (Pr) is given by eq 175, where v is the kinematic viscosity and α is the thermal diffusivity. Both of these parameters are determined from the Aspen Plus program.

$$\begin{matrix}Re = \frac{QA}{vL} & ( 174 ) \\ Pr = \frac{v}{α} & ( 175 )\end{matrix}$$

The friction factor (f) is obtained from the Pethukov correlation in eq

f=(0.79 ln(Re)−1.64)⁻² (176)

### Example 2.13

### Mathematical Model for Network Topology Optimization via Annualized Cost Minimization

Given the appropriate cost functions and heat transfer coefficients for each heat exchanger match, the superstructure of all possible topologies can be formulated based on the assigned matches. The superstructure is characterized by six distinct sets of streams: inlet (I), split (S), exchanger (E), recycle (R), mixed (M), and outlet (O). Only the conditions (flow rate and temperature) of the inlet and outlet streams are known for each heat exchanger. The remaining streams must be assigned a flow rate and temperature so that both material balances and heat balances are satisfied while preventing a temperature crossover in any of the heat exchangers.

### Example 2.14

### Mass Balances

In the following discussion, the hotstream variables are distinguished from the cold-stream variables using upper and lower case, respectively. Note that the following mathematical model is applied to each subnetwork s of the HEPN. In the general case, the superstructure must maintain mass balances at the inlet splitter (eq 177), the heat exchanger mixer (eq 178), and the heat exchanger splitter (eq 179). The mass balance for the outlet mixer is redundant information and, therefore, is not necessary.

$$\begin{matrix}{\sum }_{j ∈ {HE}_{i}^{H}}^{ } {F}_{i , j}^{S} = {F}_{i}^{H} ∀ i ∈ HOT , s ∈ SUB & ( 177 ) \\ {\sum }_{{j}^{′} ∈ {HE}_{i}^{H}}^{ {j}^{′} \neq j} {F}_{i , {j}^{′} , j}^{R} + {F}_{i , j}^{S} = {F}_{i , j}^{E} ∀ j ∈ {HE}_{i}^{H} , i ∈ HOT , s ∈ SUB & ( 178 ) \\ {\sum }_{{j}^{′} ∈ {HE}_{i}^{H}}^{ {j}^{′} \neq j} {F}_{i , j , {j}^{′}}^{R} + {F}_{i , j}^{M} = {F}_{i , j}^{E} ∀ j ∈ {HE}_{i}^{H} , i ∈ HOT , s ∈ SUB & ( 179 )\end{matrix}$$

The cold-stream balances are similar for the inlet splitter (eq 180), heat exchanger mixer (eq 181), and the heat exchanger splitter (eq 182):

$$\begin{matrix}{\sum }_{i ∈ {HE}_{j}^{c}} {f}_{j , i}^{S} = {f}_{j}^{C} \\ ∀ j ∈ COLD , s ∈ SUB & ( 180 ) \\ {\sum }_{{i}^{′} ∈ {HE}_{j}^{c}}^{{i}^{′} \neq i} {f}_{j , {i}^{′} j}^{R} + {f}_{j , i}^{S} = {f}_{j , i}^{E} \\ ∀ i ∈ {HE}_{j}^{C} , j ∈ COLD , s ∈ SUB & ( 181 ) \\ {\sum }_{{i}^{′} ∈ {HE}_{j}^{c}}^{{i}^{′} \neq i} {f}_{j , i , {i}^{′}}^{R} + {f}_{j , i}^{M} = {f}_{j , i}^{E} \\ ∀ i ∈ {HE}_{j}^{C} , j ∈ COLD , s ∈ SUB & ( 182 )\end{matrix}$$

To constrain the recycle stream in a region of interest, binary variables are introduced for the existence of the recycle streams. Using eqs 183 and 184 for the hot streams and eqs 185 and 186 for the cold streams, the hot recycle streams will be within the values F^{min} and F^{max}, while the cold recycle streams will be within the values f^{min} and f^{max}. The minimum flow rates are set to 0.1 kg/s and the maximum rates to 100 kg/s.

F_{i,j,j′}^{R}≦y_{i,j,j′}^{R,H}F^{max} (183)

F_{i,j,j′}^{R}≦y_{i,j,j′}^{R,H}F^{min} (184)

f_{j,i,i′}^{R}≦y_{j,i,i′}^{R,C}f^{max} (185)

f_{j,i,i′}^{R}≦y_{j,i,i′}^{R,C}f^{min} (186)

### Example 2.1

### Heat Balances

The hot-stream heat balances must be satisfied at the heat exchanger mixers (eq 187), the outlet mixer (eq 188), and across each heat exchanger (eq 189). Note that all enthalpy variables used are specific quantities with units (kJ/kg). The specific enthalpy is defined at the heat exchanger inlet as Q_{i,j}^{S} and at the heat exchanger outlet as Q_{i,j}^{M}. The beginning and ending enthalpy of the hot stream (Qᵢ^{Beg} and Qᵢ^{End}, respectively) are extracted from the process simulation and the total enthalpy (Q_{i,j}) is known from the minimum matches model.

$$\begin{matrix}{\sum }_{{j}^{′} ∈ {HE}_{i}^{H}}^{{j}^{′} \neq j} ( {F}_{i , {j}^{′} , j}^{R} {Q}_{i , {j}^{′}}^{M} ) + {F}_{i , j}^{S} {Q}_{i}^{Beg} = {F}_{i , j}^{E} {Q}_{i , j}^{S} \\ ∀ j ∈ {HE}_{i}^{H} , i ∈ HOT , s ∈ SUB & ( 187 ) \\ {\sum }_{j ∈ {HE}_{i}^{H}} ( {F}_{i , j}^{M} {Q}_{i , j}^{M} ) = {F}_{i}^{H} {Q}_{i}^{End} \\ ∀ i , ∈ HOT , s ∈ SUB & ( 188 ) \\ {F}_{i , j}^{E} ( {Q}_{i , j}^{S} - {Q}_{i , j}^{M} ) = {Q}_{i , j} \\ ∀ j ∈ {HE}_{i}^{H} , i ∈ HOT , s ∈ SUB & ( 189 )\end{matrix}$$

The cold stream balances are similar for the heat exchanger mixers (eq 190), the outlet mixer (eq 191), and the heat exchangers (eq 192). The naming convention of the cold-stream enthalpy variables is similar to that used for the hot streams, but with lower case letters being used to distinguish between the two sets.

$$\begin{matrix}{\sum }_{{i}^{′} ∈ {HE}_{j}^{C}}^{{i}^{′} \neq i} ( {f}_{j , {i}^{′} , i}^{R} {q}_{j , {i}^{′}}^{M} ) + {f}_{j , i}^{S} {q}_{j}^{Beg} = {f}_{j , i}^{E} {q}_{j , i}^{S} \\ ∀ i ∈ {HE}_{j}^{C} , j ∈ COLD , s ∈ SUB & ( 190 ) \\ {\sum }_{i ∈ {HE}_{j}^{C}} ( {f}_{j , i}^{M} {q}_{j , i}^{M} ) = {f}_{j}^{C} {q}_{j}^{End} \\ ∀ j ∈ COLD , s ∈ SUB & ( 191 ) \\ {f}_{j , i}^{E} ( {q}_{j , i}^{M} - {q}_{j , i}^{S} ) = {Q}_{i , j} \\ ∀ i ∈ {HE}_{j}^{C} , \\ j ∈ COLD , s ∈ SUB & ( 192 )\end{matrix}$$

To relate the stream enthalpy to the appropriate temperature, binary variables are utilized, based on the heat capacities used in the previous models. That is, the heat profile can be determined for the hot stream (Q_{i,k}^{Prof}) and the cold stream (q_{j,k}^{Prof}), which represents the cumulative amount of heat delivered by the stream by the end of interval k. These values represent bounds on the value of the enthalpy flow rate for a given stream if it exists in a particular temperature interval. Thus, the binary variables y_{i,k}^{H} and y_{j,k}^{C} can be used to pinpoint the appropriate temperature interval for the heat exchanger inlet (see eqs 193 and 194 for the hot stream and eqs 195 and 196 for the cold stream) and for the heat exchanger outlet (eqs 197 and 198 for the hot stream and eqs 199 and 200 for the cold stream).

$$\begin{matrix}{F}_{i , j}^{E} {Q}_{i , j}^{S} \leq {\sum }_{k ∈ TI} {y}_{i , k}^{H , S} {Q}_{i , k + 1}^{Prof} \\ ∀ j ∈ {HE}_{i}^{H} , i ∈ HOT , s ∈ SUB & ( 193 ) \\ {F}_{i , j}^{E} {Q}_{i , j}^{S} \geq {\sum }_{k ∈ TI} {y}_{i , k}^{H , S} {Q}_{i , k}^{Prof} \\ ∀ j ∈ {HE}_{i}^{H} , i ∈ HOT , s ∈ SUB & ( 194 ) \\ {f}_{j , i}^{E} {q}_{j , i}^{S} \leq {\sum }_{k ∈ TI} {y}_{j , k}^{C , S} {q}_{j , k + 1}^{Prof} \\ ∀ i ∈ {HE}_{j}^{C} , j ∈ COLD , s ∈ SUB & ( 195 ) \\ {f}_{j , i}^{E} {q}_{j , i}^{S} \geq {\sum }_{k ∈ TI} {y}_{j , k}^{C , S} {q}_{j , k}^{Prof} \\ ∀ i ∈ {HE}_{j}^{C} , j ∈ COLD , s ∈ SUB & ( 196 ) \\ {F}_{i , j}^{E} {Q}_{i , j}^{M} \leq {\sum }_{k ∈ TI} {y}_{i , k}^{H , M} {Q}_{i , k + 1}^{Prof} \\ ∀ j ∈ {HE}_{i}^{H} , i ∈ HOT , s ∈ SUB & ( 197 ) \\ {F}_{i , j}^{E} {Q}_{i , j}^{M} \geq {\sum }_{k ∈ TI} {y}_{i , k}^{H , M} {Q}_{i , k}^{Prof} \\ ∀ j ∈ {HE}_{i}^{H} , i ∈ HOT , s ∈ SUB & ( 198 ) \\ {f}_{j , i}^{E} {q}_{j , i}^{M} \leq {\sum }_{k ∈ TI} {y}_{j , k}^{C , M} {q}_{j , k + 1}^{Prof} \\ ∀ i ∈ {HE}_{j}^{C} , j ∈ COLD , s ∈ SUB & ( 199 ) \\ {f}_{j , i}^{E} {q}_{j , i}^{M} \geq {\sum }_{k ∈ TI} {y}_{j , k}^{C , M} {q}_{j , k}^{Prof} \\ ∀ i ∈ {HE}_{j}^{C} , j ∈ COLD , s ∈ SUB & ( 200 )\end{matrix}$$

### Example 2.16

### Temperature Constraints

Logical constraints are used to refer to only one temperature interval for the hot inlet (eq 201), the hot outlet (eq 202), the cold inlet (eq 203), and the cold outlet (eq 204).

$$\begin{matrix}{\sum }_{k ∈ TI} {y}_{i , k}^{H , S} = 1 \\ ∀ i ∈ HOT , s ∈ SUB & ( 201 ) \\ {\sum }_{k ∈ TI} {y}_{i , k}^{H , M} = 1 \\ ∀ i ∈ HOT , s ∈ SUB & ( 202 ) \\ {\sum }_{k ∈ TI} {y}_{j , k}^{C , S} = 1 \\ ∀ j ∈ COLD , s ∈ SUB & ( 203 ) \\ {\sum }_{k ∈ TI} {y}_{j , k}^{C , M} = 1 \\ ∀ j ∈ COLD , s ∈ SUB & ( 204 )\end{matrix}$$

The temperature of the streams is linearly dependent on the enthalpy of the temperature interval, because the heat capacity is assumed to be constant within the interval. Using the temperature values T_{i,k}^{Prof} and t_{j,k}^{Prof}, which correspond to the temperature intervals, the inlet and outlet temperatures can be defined using eqs 205-208. Note that the heat capacity values are the same as in the previous mathematical models and, therefore, these equations are linear.

$$\begin{matrix}{T}_{i , j}^{S} = \frac{1}{{C}_{i , k}^{H}} ( {Q}_{i , j}^{S} - {Q}_{i , k}^{Prof} ) - {y}_{i , k}^{H , S} {T}_{i , k}^{Prof} \\ ∀ j ∈ {HE}_{i}^{H} , ( i , k ) ∈ {HOT}_{s} , s ∈ SUB & ( 205 ) \\ {T}_{i , j}^{M} = \frac{1}{{C}_{i , k}^{H}} ( {Q}_{i , j}^{M} - {Q}_{i , k}^{Prof} ) - {y}_{i , k}^{H , M} {T}_{i , k}^{Prof} \\ ∀ j ∈ {HE}_{i}^{H} , ( i , k ) ∈ {HOT}_{s} , s ∈ SUB & ( 206 ) \\ {t}_{j , i}^{S} = \frac{1}{{C}_{j , k}^{C}} ( {q}_{j , i}^{S} - {q}_{j , k}^{Prof} ) - {y}_{j , k}^{C , S} {t}_{j , k}^{Prof} \\ ∀ i ∈ {HE}_{j}^{C} , ( j , k ) ∈ {COLD}_{s} , s ∈ SUB & ( 207 ) \\ {t}_{j , i}^{M} = \frac{1}{{C}_{j , k}^{C}} ( {q}_{j , i}^{M} - {q}_{j , k}^{Prof} ) - {y}_{j , k}^{C , M} {t}_{j , k}^{Prof} \\ ∀ i ∈ {HE}_{j}^{C} , ( j , k ) ∈ {COLD}_{s} , s ∈ SUB & ( 208 )\end{matrix}$$

Temperature crossover within the heat exchangers is prevented using eqs 209 and 210. The minimum temperature approach (Tₘᵢₙ) was set to 0.1° C. in this study.

T_{i,j}^{M}−t_{j,i}^{S}≧Tₘᵢₙ∀j∈HEᵢ^{H},i∈HOT,s∈SUB (209)

T_{i,j}^{S}−t_{j,i}^{M}≧Tₘᵢₙ∀j∈HEᵢ^{H},i∈HOT,s∈SUB (210)

The area associated with a heat exchanger is calculated using eq 211, where the log-mean temperature difference (LMTD) is defined in eq 214. The Patterson approximation is used for LMTD to circumvent the computational difficulty associated with very small temperature approaches.

$$\begin{matrix}{A}_{i , j} = \frac{{Q}_{i , j}}{{U}_{i , j} L M T {D}_{i , j}} & ( 211 ) \\ Δ {T}_{i , j}^{1} = {T}_{i , j}^{M} - {t}_{j , i}^{S} & ( 212 ) \\ Δ {T}_{i , j}^{2} = {T}_{i , j}^{S} - {t}_{j , i}^{M} & ( 213 ) \\ L M T {D}_{i , j} = \frac{2}{3} {( Δ {T}_{i , j}^{1} + Δ {T}_{i , j}^{2} )}^{1 / 2} + \frac{1}{6} ( Δ {T}_{i , j}^{1} + Δ {T}_{i , j}^{2} ) \\ ∀ j ∈ {HE}_{i}^{H} , i ∈ {HOT}_{s} , s ∈ SUB & ( 214 )\end{matrix}$$

Objective. The objective is then given by eq 215,

$${min}_{F , f , T , t , A} {\sum }_{( i , j ) ∈ HE} {C}_{o , i , j} {A}_{i , j}^{{f}_{i , j}} \qquad ( 215 )$$

where the cost (C_{o,i,j}) and scaling (f_{i,j}) parameters were determined using the annualized cost calculation described previously.

Equations 177-215 represent a nonconvex mixed-integer nonlinear optimization problem (MINLP) that can be solved using DICOPT20 with the nonlinear solver CONOPT21 and the mixed-integer solver CPLEX.13 The minimum superstructure is designed for each subnetwork1, by eliminating impossible connections, using known information about the stream temperatures for each match. Two hundred (200) initial points are selected by assuming no recycle flow and different split fractions at the inlet, and the topology with the smallest annualized cost is selected as the final structure. The selection of multiple initial points is based on having nonconvex MINLP models for which local MINLP solvers (e.g., DICOPT) are employed.

### Example 2.17

### Computational Results and Illustrative Examples

The overall results for the annualized cost model are presented in Table 31. The total annualized cost for each subnetwork was normalized by the amount of products formed to facilitate a proper comparison. From Table 31, the largest annualized investment cost is $3.288/bbl and all seven process flowsheets are within a range of $0.858/bbl to each other. Furthermore, this investment cost is, with regard to magnitude, about one-third to one-fifth of the cost of the HEPN utilities that are recovered in the Minimum Hot/Cold/Power Utilities model. This serves to validate the decomposition of the HEPN problem into subtasks. The total annualized HEPN cost is also shown in Table 31 and is indicative of the cost benefit of only the HEPN. That is, the electricity associated with the electrolyzers and compressors in Table 25 is not included in this cost. Note that, in all seven cases, the HEPN serves to reduce the total cost of the final products. This is not surprising, because a large amount of electricity is recovered from the Minimum Hot/Cold/Power Utilities model, which helps avoid the purchase of a large quantity of this power source.

TABLE 31
Minimum Annualized Cost for the CBGTL Process Alternatives

| | | | | | | |
|---|---|---|---|---|---|---|
| C-R-A | C-E-A | B-R-A | B-E-A | H-R-A | H-E-A | H-R-T |

Annualized Investment Cost (2009 $/bbl)

| | | | | | | | |
|---|---|---|---|---|---|---|---|
| Subnetwork 1 | 0.288 | 0.390 | 0.420 | 0.366 | 0.414 | 0.366 | 0.312 |
| Subnetwork 2 | 1.308 | 1.062 | 1.332 | 0.810 | 1.758 | 0.966 | 0.444 |
| Subnetwork 3 | 1.074 | 1.230 | 1.086 | 1.542 | 1.116 | 1.440 | 1.674 |
| Total | 2.670 | 2.682 | 2.838 | 2.718 | 3.288 | 2.772 | 2.430 |

Annual HEPN Utility Cost (2009 $/bbl)

| | | | | | | |
|---|---|---|---|---|---|---|
| −8.110 | −6.446 | −6.720 | −9.241 | −8.090 | −8.489 | −12.772 |

Annualized HEPN Cost (2009 $/bbl)

| | | | | | | |
|---|---|---|---|---|---|---|
| −5.440 | −3.764 | −3.882 | −6.523 | −4.802 | −5.717 | −10.342 |

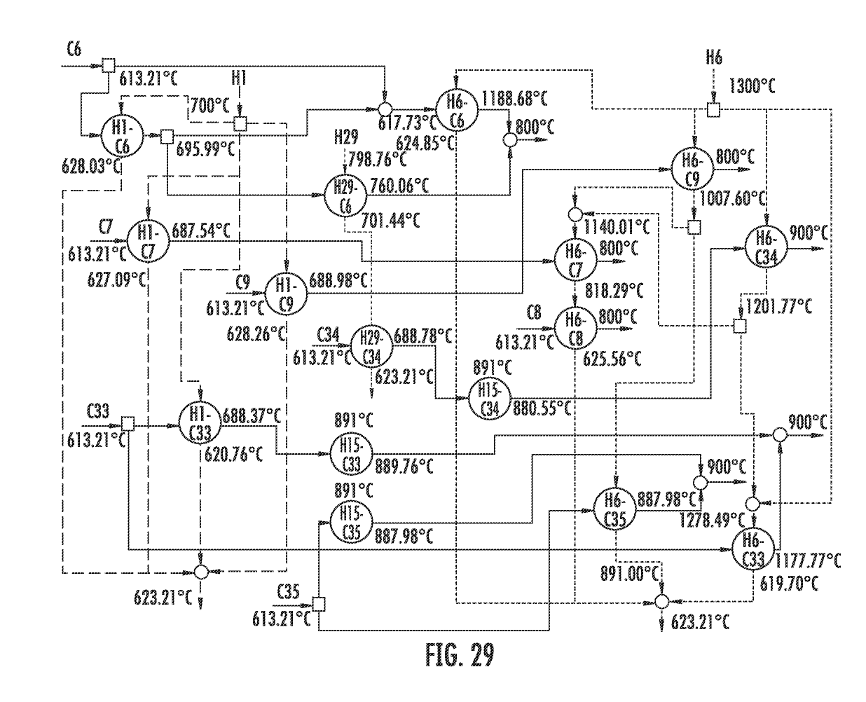

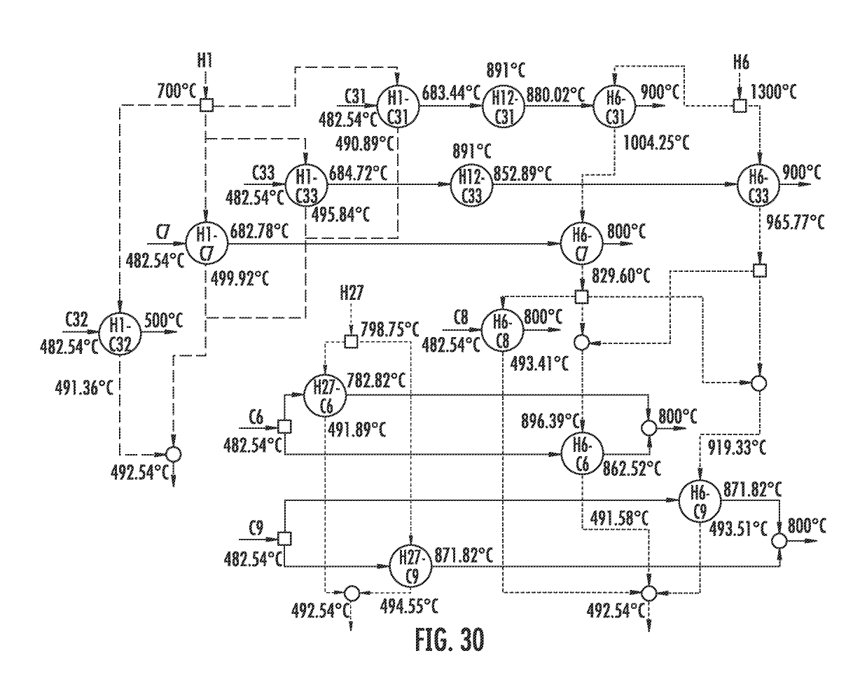

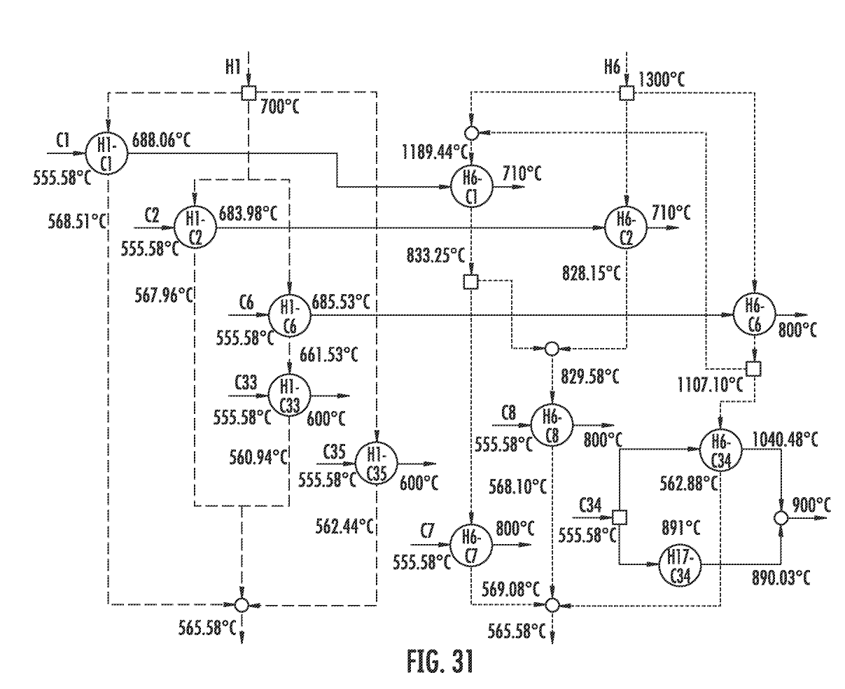

To further illustrate the results of the mathematical model, the topology of subnetwork 1 for each of the hybrid process flowsheets is shown in FIGS. 29, 30, and 31 for H-R-A, HE-A, and H-R-T, respectively. Also included in the figures are the inlet and outlet temperatures of both the hot and cold streams for each match. For clarity, the hot streams are included as dashed lines, whereas the cold streams are solid lines. The streams present in these figures include the reverse water-gasshift (RGS) effluent (H1), the fuel combuster effluent (H6), a heat engine precooler (H29 for H-R-A, H27 for H-E-A), the RGS inlet hydrogen (C1), the RGS recycle CO₂ (C2), the autothermal reactor (ATR) steam input (C6), the ATR natural gas input (C7), the ATR oxygen input (C8), the ATR recycle light gas input (C9), and the heat engine superheaters (C33-C35 for H-R-T and H-R-T, C31-C33 for H-E-A). Also included is the coal gasifier (H15 for H-R-A and H-R-T; H12 for H-E-A). Note that the coal gasifier will not have corresponding streams, because it is a point source of heat. The temperature for the coal gasifier remains constant at 891° C. and is shown in italic font in the figures.

A few key differences between the process topologies are immediately obvious. Note that the pinch points for each of these subnetworks are different, so the topologies are expected to be different. Furthermore, the operating conditions of the heat engines will be different for each flowsheet, so it is not expected that the same number of heat engine streams will be present in each subnetwork. In fact, we only see a hot precooler stream for the H-R-A and H-E-A subnetworks, because the turbine outlet temperatures of all three heat engines for the H-R-T subnetwork fall below the pinch point associated with this subnetwork. Another difference is the presence of cold streams C1 and C2 (inlets to the RGS reactor) in the H-R-T subnetwork but not in the H-R-A or H-E-A subnetwork. A design specification in the CBGTL flowsheets was to vary the input temperature of the RGS input streams, to provide the necessary heat duty of reaction. This serves to supplement oxygen input to the unit and helps reduce the hydrogen requirement of the flowsheet. In the H-R-T flowsheet, the RGS inlet streams were preheated to 710° C. and were thus included in the high temperature subnetwork. The H-R-A and H-E-A RGS inlet streams were heated to 472.16 and 473.26° C., respectively, and thus were not considered in the high temperature subnetwork.

Although the topologies are distinctly different, there are several similarities to note. The ATR unit has each of the feed streams preheated to 800° C. to reduce the oxygen requirement needed to provide the heat of reaction. Because of the restrictions placed on matches between point sources and process streams, none of these preheated streams extracts heat from the coal gasifier. Rather, a combination of the fuel combuster, heat engine precoolers, and RGS effluent provides the necessary heat. Furthermore, the only streams that interact with the coal gasifier are the three heat engine superheaters. A second major similarity is that most of the cold streams interact with the RGS effluent and then the fuel combustor. This is expected due to the higher temperature of the fuel combustor effluent (1300° C., compared to 700° C.). It is finally worth noting that, although the minimum allowed temperature approach of the streams was 0.1° C., the minimum value that is seen in the figures is 1° C. This prevents the LMTD value of a given match from becoming very small and thus increasing the area of the heat exchanger to large values.

A new framework for simultaneous heat and power integration for the coal, biomass, and natural gas to liquids (CBGTL) process is disclosed. This was done using a three-stage decomposition where the minimum hot/cold/power utility cost, the minimum number of heat exchanger matches, and the minimum annualized cost of heat exchange were sequentially calculated. A superset of possible heat engines were introduced to produce electricity, using the waste heat from the process streams. The minimum hot/cold/power utility model found the set of operating conditions of the heat engines that can recover the most electricity while explicitly taking into account interaction with the entire process flowsheet and the necessary cooling water requirement. Using the results of the minimum utility model, the minimum matches model utilized both weighted matches and vertical heat transfer to distinguish between solutions with the same number of heat exchanger matches. Weights were assigned to a given set of streams based on their proximity in the plant, as well as the relative flow rates of the streams. The optimal set of heat exchanger matches along with the heat load of each match was directly transferred to the minimum annualized cost model to find the optimal heat exchanger topology. Explicit formulas were derived for the annualized cost functions, assuming that each heat exchanger would either be a floating-head unit or a kettle reboiler and overall heat transfer coefficients were estimated for every heat exchanger. The results of the annualized cost model provided heat transfer areas for each exchanger, which could then be directly utilized in an economic analysis.

### Example 3

### Process Synthesis of Hybrid Coal, Biomass, and Natural Gas to Liquids Via Fischer-Tropsch Synthesis, ZSM-5 Catalytic Conversion, Methanol Synthesis, Methanol-to-Gasoline, and Methanol-to-Olefins/Distillate Technologies

Several technologies for synthesis gas (syngas) refining are introduced into a thermochemical based superstructure that will convert biomass, coal, and natural gas to liquid transportation fuels using Fischer Tropsch (FT) synthesis or methanol synthesis. The FT effluent can be (i) refined into gasoline, diesel, and kerosene or (ii) catalytically converted to gasoline and distillate over a ZSM-5 zeolite. Methanol can be converted using ZSM-5 (i) directly to gasoline or to (ii) distillate via olefin intermediates. A mixed integer nonlinear optimization model that includes simultaneous heat, power, and water integration is solved to global optimality to determine the process topologies that will produce the liquid fuels at the lowest cost. Twenty-four case studies consisting of different (a) liquid fuel combinations, (b) refinery capacities, and (c) superstructure possibilities are analyzed to identify important process topological differences and their effect on the overall system cost, the process material/energy balances, and the well-to-wheel greenhouse gas emissions.

The disclosure herein introduces several distinct methods for conversion of syngas to liquid fuels into the CBGTL process superstructure and investigates the tradeoffs that arise from these methods. The superstructure in Examples 1 and 2 converted the syngas into a raw FT hydrocarbon product using one of four FT units operating with either a cobalt or iron catalyst and at high or low temperature. The effluent was subsequently fractionated and upgraded using a series of hydrotreating units, a wax hydrocracker, two isomerization units, a naphtha reformer, an alkylation unit, and a gas separation plant (i.e., deethanizer).

This example introduces two iron-based FT units that utilize the forward water-gas-shift reaction to produce the raw hydrocarbons using an input H₂/CO ratio that is less than the typical 2/1 ratio needed for FT synthesis. Catalytic conversion of the FT vapor effluent over a ZSM-5 catalyst is considered as an alternative for producing gasoline range hydrocarbons from the raw FT effluent.

Methanol synthesis and subsequent conversion to liquid hydrocarbons are also introduced into the superstructure. The methanol may be catalytically converted using a ZSM-5 zeolite to (i) gasoline range hydrocarbons or (ii) to distillate (i.e., diesel and kerosene) via an intermediate coversion to olefins. The mathematical modeling and cost functions needed to incorporate the above alternatives into the superstructure are outlined in detail. The complete process synthesis optimization model is then tested on a total of 24 case studies which consist of two liquid product combinations, three plant capacities, and four plant superstructures. Using low-volatile bituminous coal (Illinois #6) and perennial biomass (switchgrass), important topological differences between the case studies are discussed and the results of each component of the process synthesisframework are illustrated.

### Example 3.1

### CBGTL Mathematical Model for Process Synthesis with Simultaneous Heat, Power, and Water Integration

This example will discuss the enhancements to the previous mathematical model for process synthesis and simultaneous heat, power, and water integration that will incorporate a wide variety of designs for syngas conversion and hydrocarbon upgrading. Modeling of these enhancements will be described in detail in the following section and the complete mathematical model is listed in Example 3.15. The nomenclature used in the mathematical description below is outlined in Table 32, below. Note that this table represents a subset of the comprehensive list of symbols that are needed for the full mathematical model. The full list of symbols and mathematical model are included for reference in Example 3.15.

TABLE 32
Mathematical model nomenclature

| Symbol | Definition |
|---|---|
| Indices | |
| s | Species index |
| u | Process unit index |
| Sets | |
| (u, u′) | Stream from unit u to unit u′ |
| (u, u′, s) | Species s within stream (u, u′) |
| u ε U_{FT}^{lr} | Set of all iron-based FT units |
| Parameters | |
| Kᵤ^{WGS} | Water-gas-shift equilibrium constant for unit u |
| Kᵤ^{MSN} | Methanol synthesis equilibrium constant for unit u |
| Variables | |
| N_{u,u′,s}^{S} | Molar flow of species s from unit u to unit u′ |
| x_{u,u′,s}^{S} | Molar concentration of species s from unit u to unit u′ |

### Example 3.2

### Conceptual Design

The syngas conversion and hydrocarbon upgrading units proposed herein is based on an extension of the CBGTL refinery superstructure in Examples 1 and 2. All relevant thermodynamic information (i.e., chemical equilibrium constants, vapor-liquid equilibrium constants, specific enthalpies, and heat capacities) for the units and streams in the refinery have been extracted from Aspen Plus v7.3 using the Peng-Robinson equation of state with the Boston-Mathias alpha function. The flowsheets depicting the extensions of the superstructure are shown in FIGS. 32-36 of and the complete superstructure is included. In the figures, fixed process units are represented by 110, variable process units by 120, splitter units by 130, and mixer units by 140. The variable process streams are represented by 210 and all other process streams are fixed, unless otherwise indicated. Note that some units (e.g., compressors, pumps, heat exchangers) are not included in the figures for clarity, though these units are thoroughly modeled in the CBGTL refinery.

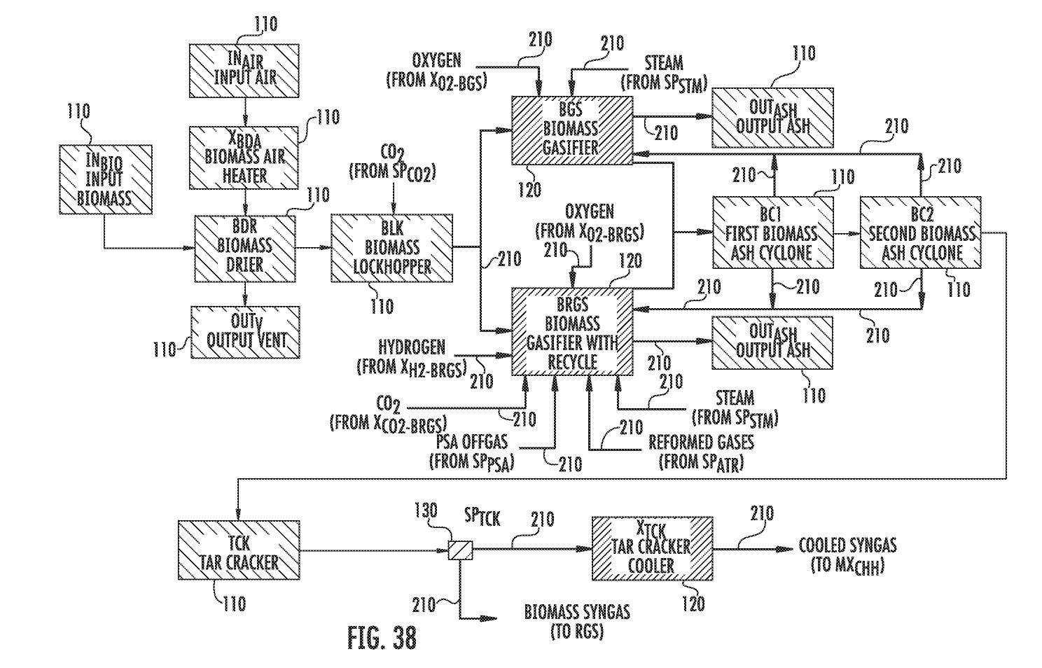

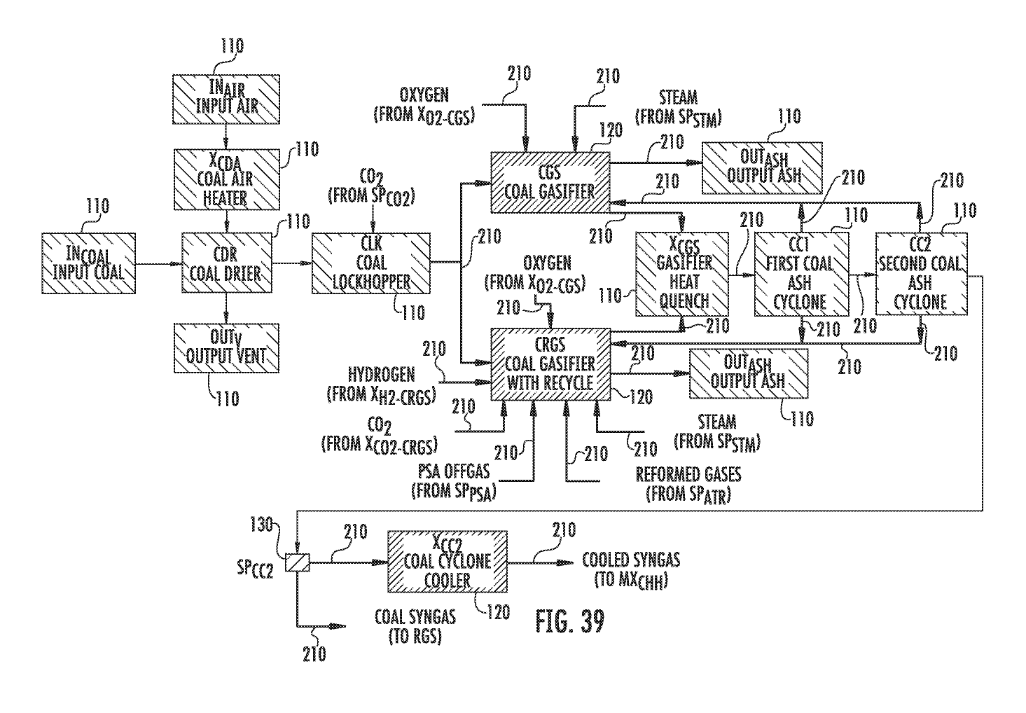

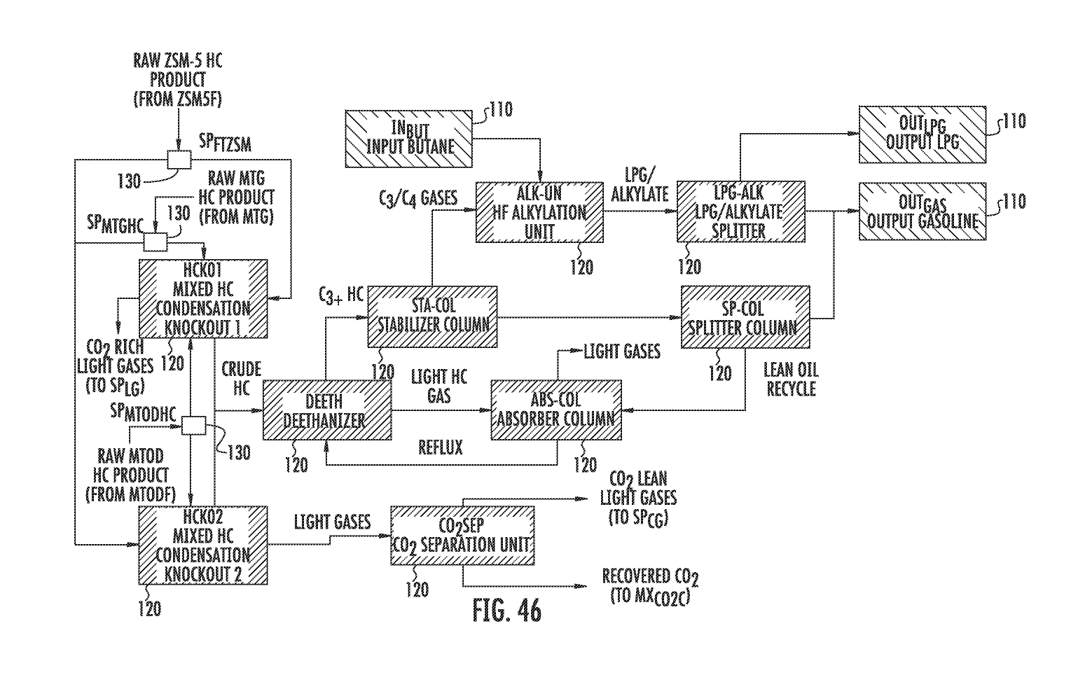

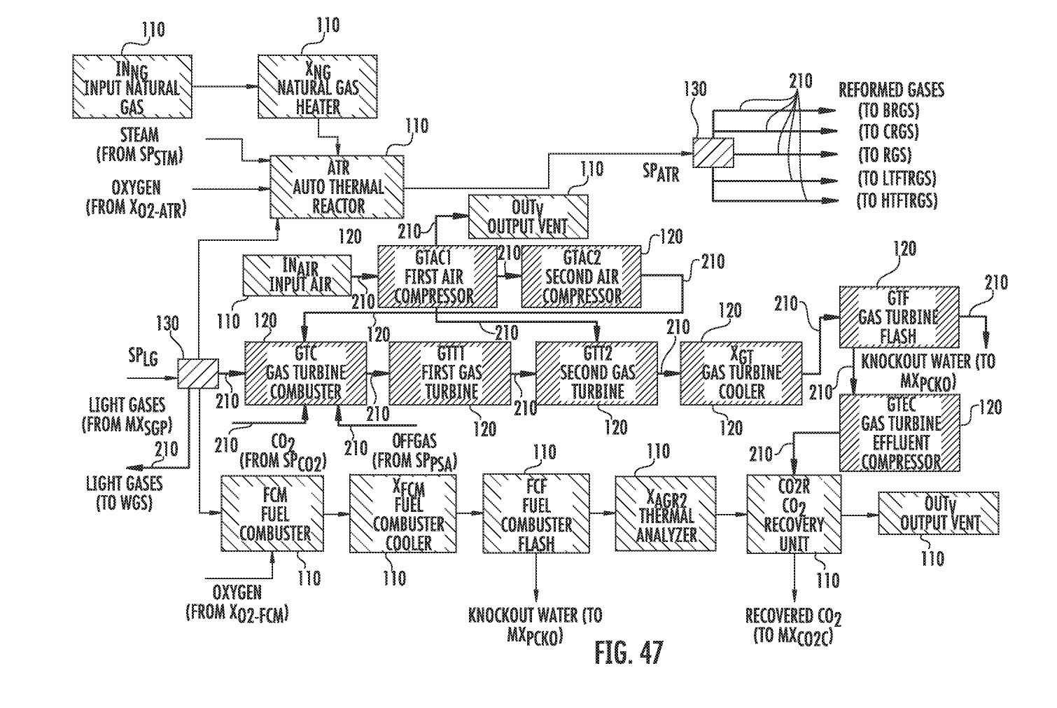

The CBGTL superstructure is designed to co-feed biomass, coal, or natural gas to produce gasoline, diesel, and kerosene. Syngas is generated via gasification from biomass (FIG. 38) or coal (FIG. 39) or auto-thermal reforming of natural gas (FIG. 47). Co-feeding of the coal, biomass, or natural gas in a single gasifier unit was not considered in this study due to the lack of (i) technical maturity of the process design and (ii) cost and operating data for co-fed units. Synergy for co-fed biomass and coal gasification and simultaneous reforming the natural gas using the gasifier quench heat (Adams & Barton, 2011, which is incorporated herein by reference as if fully set forth) may be important to reduce the capital cost required for synthesis gas production, and the authors note that the optimization model is capable of including the technoeconomic benefit of co-fed gasification if cost and operational data become available.

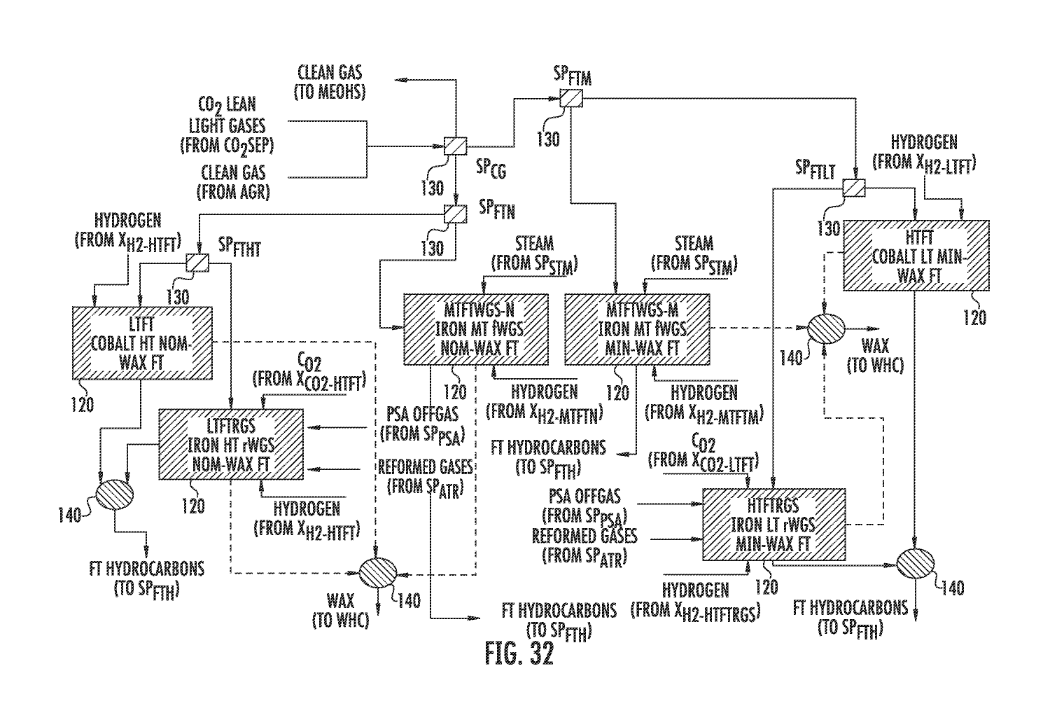

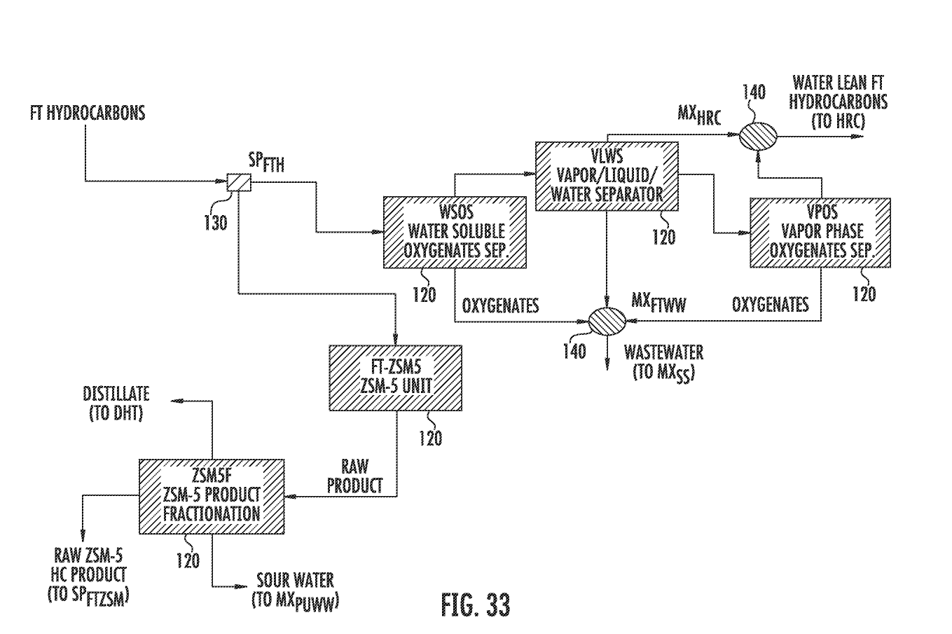

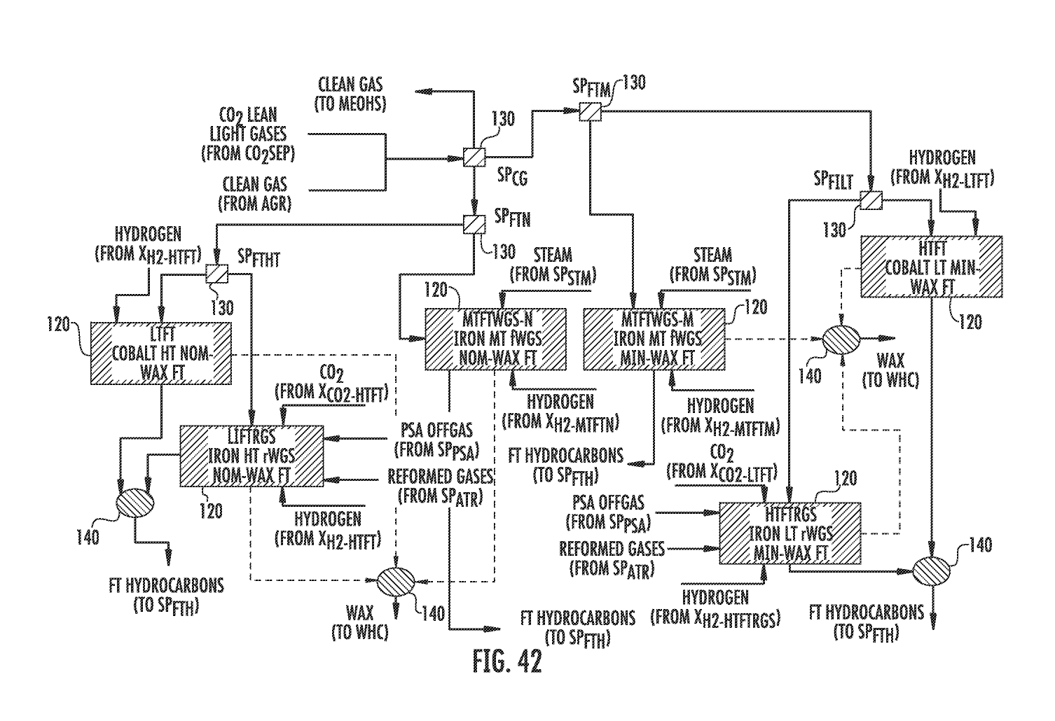

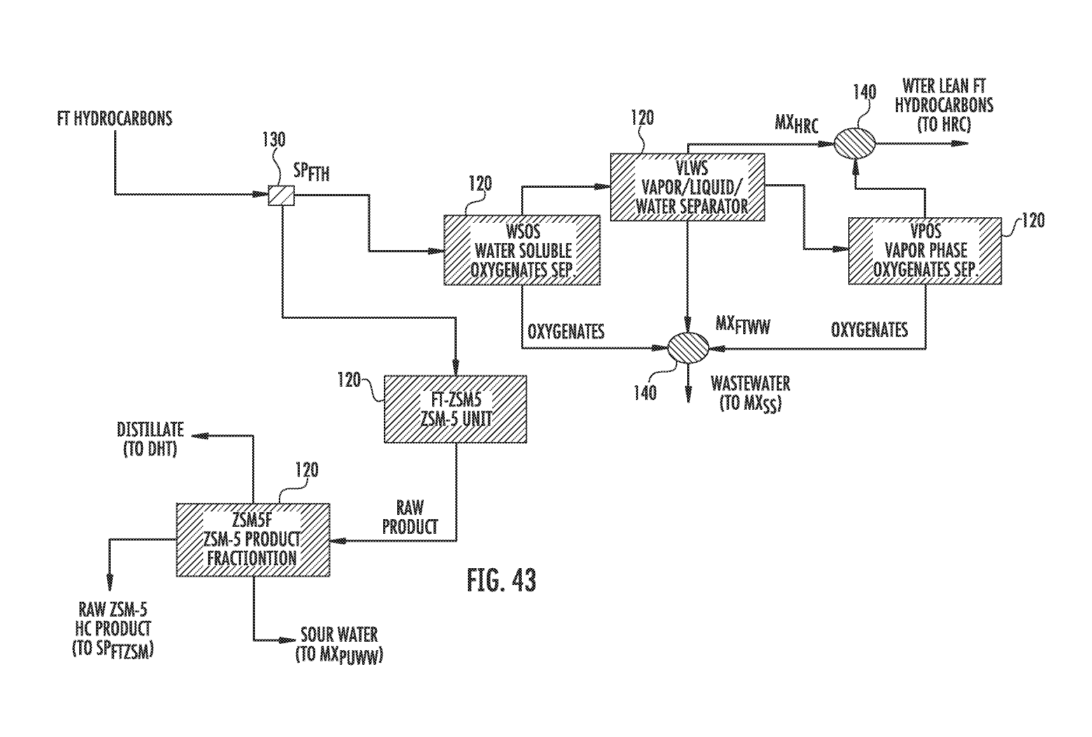

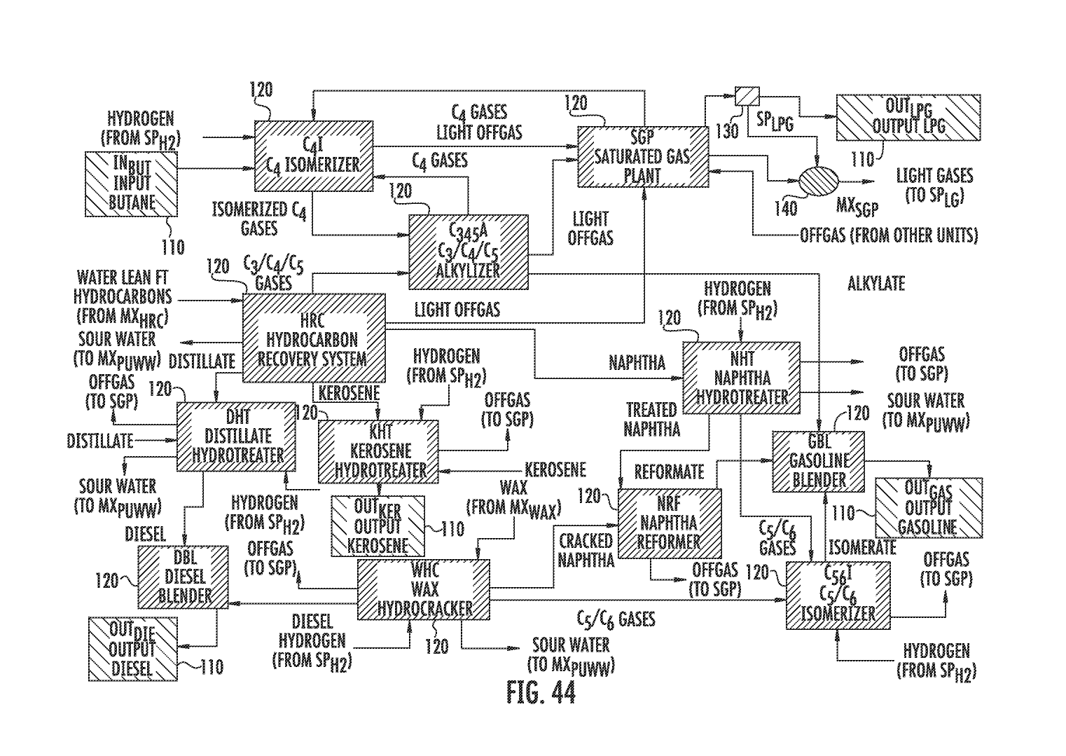

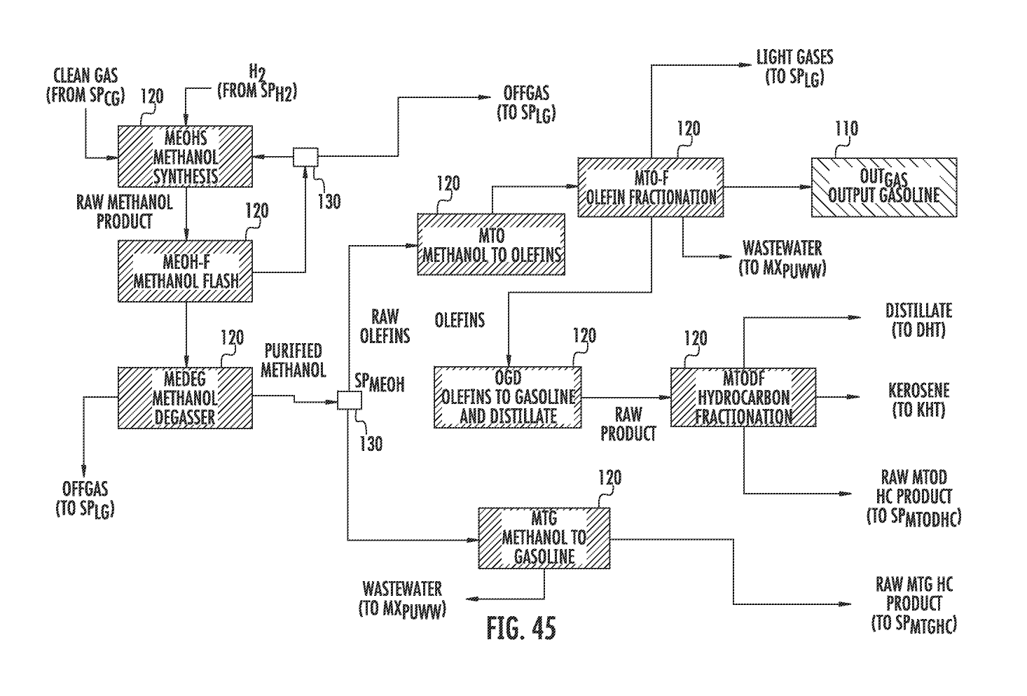

The synthesis gas is either (i) converted into hydrocarbon products in the Fischer-Tropsch (FT) reactors (FIG. 32; FIG. 42) or (ii) into methanol via methanol synthesis (FIG. 35; FIG. 45). The FT wax will be sent to a hydrocracker to produce distillate and naphtha (FIG. 44) while the FT vapor effluent may be (a) fractionated and upgraded into gasoline, diesel, or kerosene or (FIG. 43; FIG. 44) (b) catalytically converted to gasoline via a ZSM-5 zeolite (FIG. 33; FIG. 43). The methanol may be either (a) catalytically converted to gasoline via the ZSM-5 catalyst (FIGS. 35-36; FIGS. 45-46) or (b) catalytically converted to olefins via the ZSM-5 catalyst and subsequently fractionated to distillate and gasoline (FIG. 35; FIG. 45).

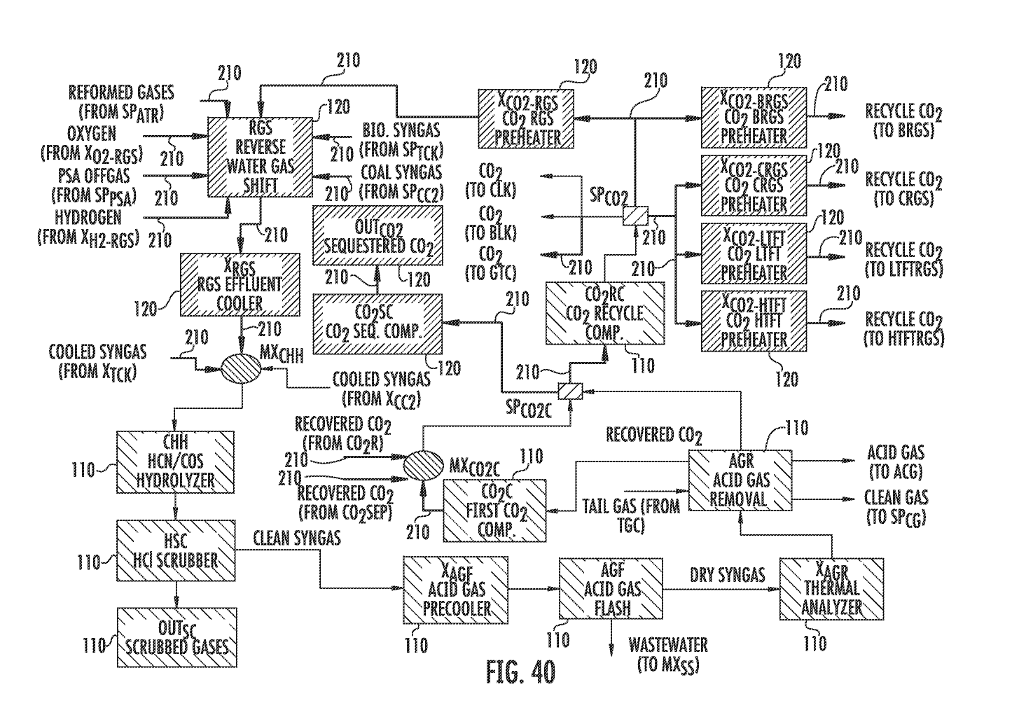

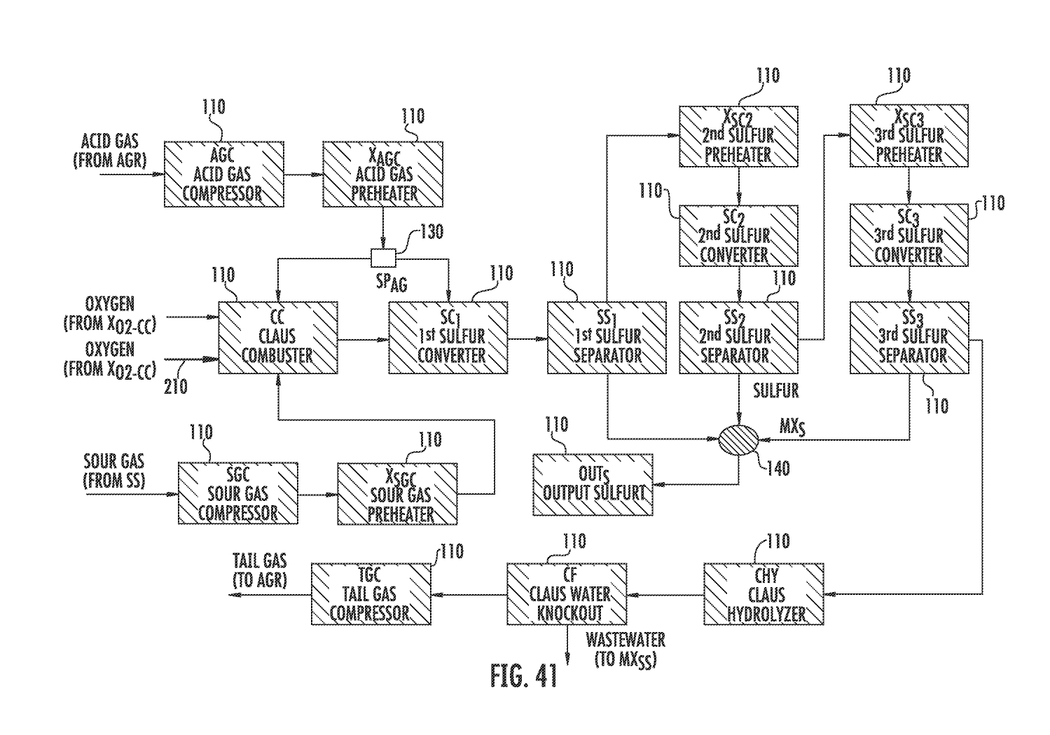

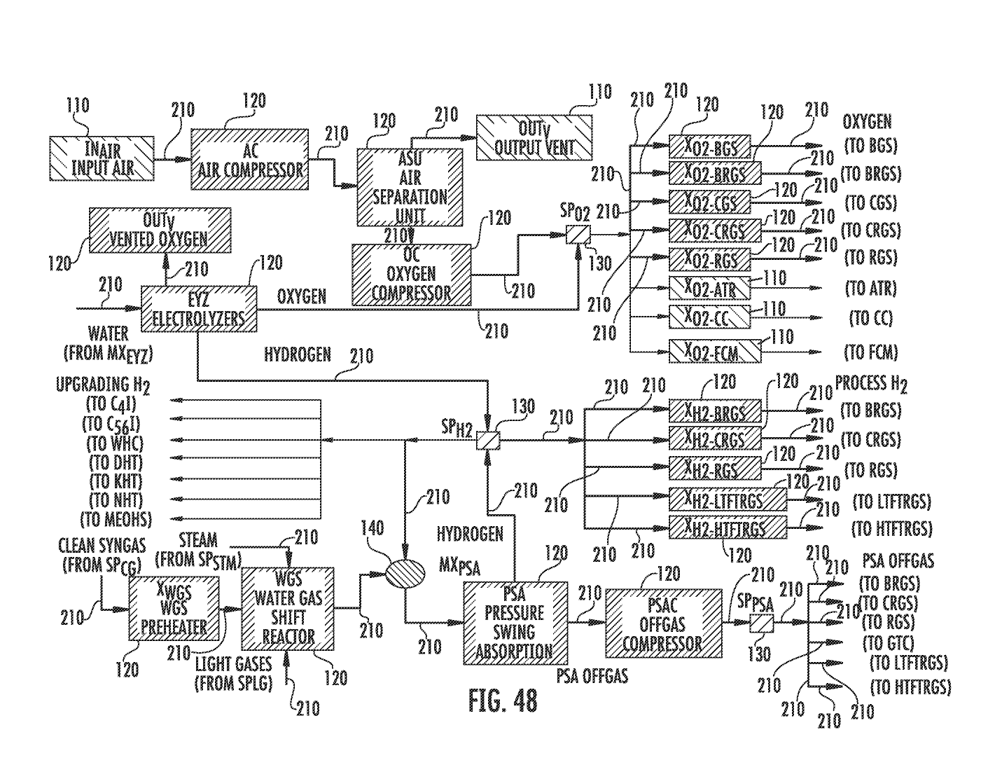

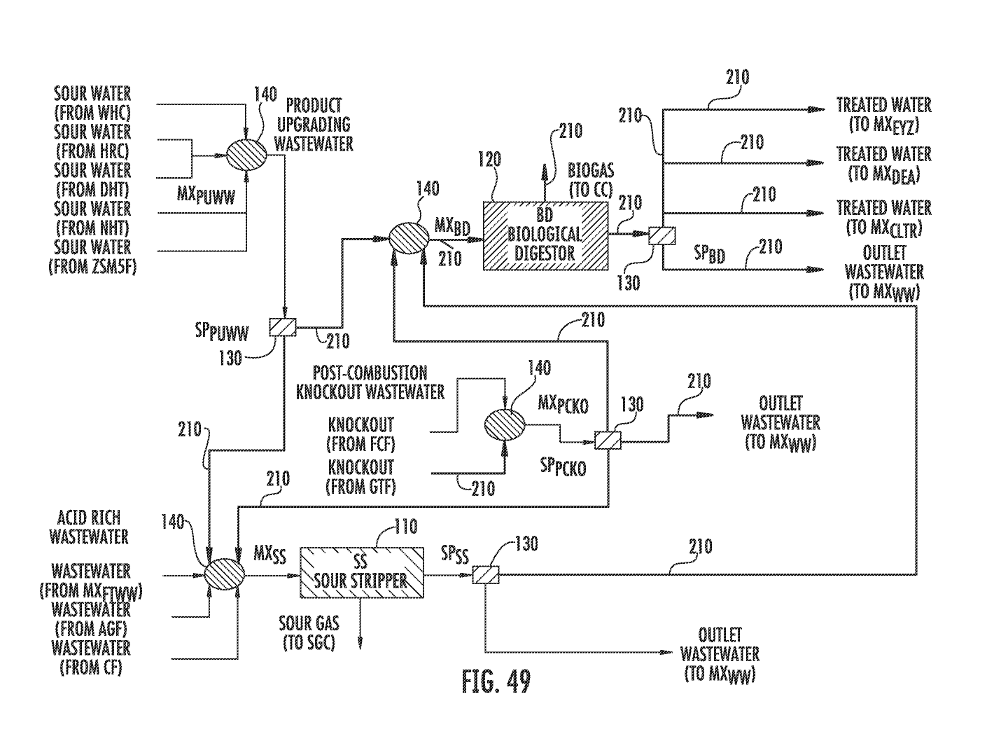

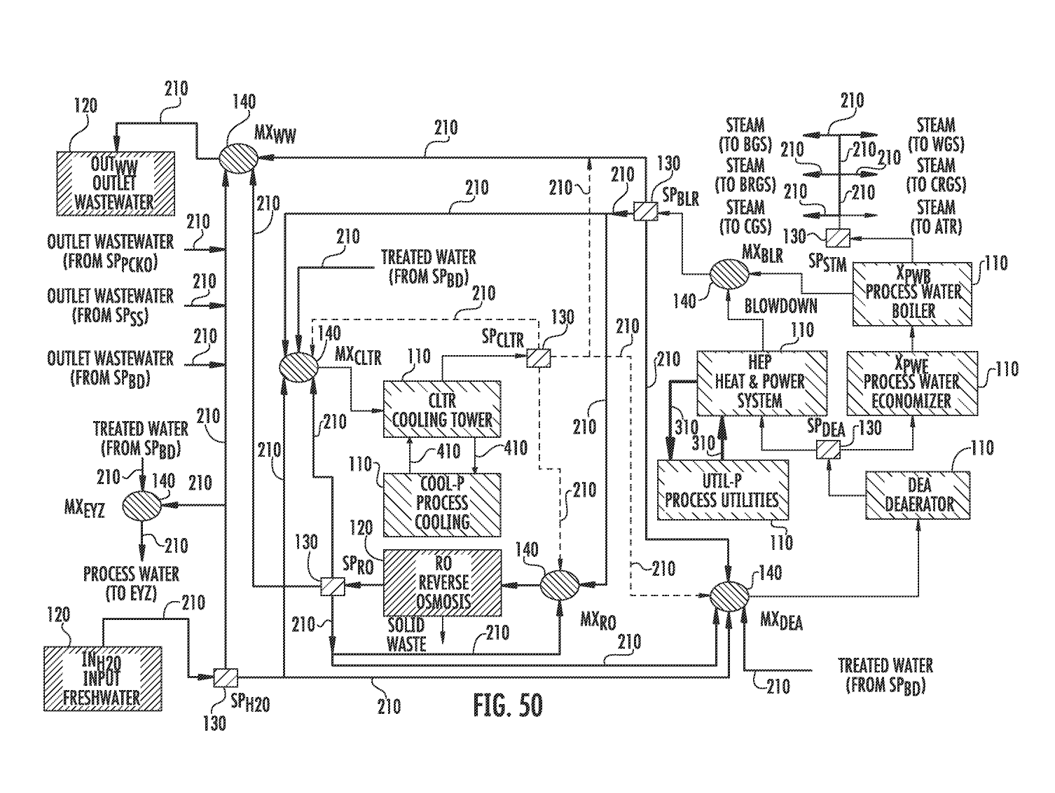

Acid gases including CO₂, H₂, and NH₃ are removed from the syngas via a Rectisol unit prior to conversion to hydrocarbons or methanol (FIG. 40). Incorporation of other acid gas removal technologies (e.g., amine adsorption, pressure-swing adsorption, vacuum-swing adsorption, membrane separation) and their relative capital/operating cost as a function of input flow rate and acid gas concentration is the subject of an ongoing study. The sulfur-rich gases are directed to a Claus recovery process (FIG. 41) and the recovered CO₂ may be sequestered (FIG. 40) or reacted with H₂ via the reverse water-gas-shift reaction. The CO₂ may be directed to either the gasifiers (FIGS. 38-39), the reverse water gas-shift reactor (FIG. 40), or the iron-based FT units (FIG. 42). Recovered CO₂ is not sent to the cobalt-based FT units to ensure a maximum molar concentration of 3% and prevent poisoning of the catalyst. Hydrogen is produced via pressure-swing adsorption or an electrolyzer unit while oxygen can be provided by the electrolyzer or a distinct air separation unit (FIG. 48). A complete water treatment network (FIGS. 49-50) is incorporated that will treat and recycle wastewater from various process units, blowdown from the cooling tower, blowdown from the boilers, and input freshwater. Clean output of the network includes (i) process water to the electrolyzers, (ii) steam to the gasifiers, autothermal reactor, and water-gas-shift reactor, and (iii) discharged wastewater to the environment.

The effluent of each reactor in the CBGTL refinery is based on either (i) known extents of reaction, (ii) thermodynamically limited equilibrium, or (iii) a specified composition from a literature source. Reaction system (i) is used in the gasifiers, the tar cracker, and the combustor units (e.g., fuel combustor, gas turbine, Claus combustor) and the extents of reaction are based on known information from literature (gasifiers/cracker) or from the operating conditions of the unit (i.e., complete combustion using a stoichiometric excess of oxygen). Reaction system (ii) is used for the water-gas-shift reaction (i.e., gasifiers, WGS reactor, FT units, methanol synthesis, auto-thermal reactor), methanol synthesis, and steam reforming in the auto-thermal reactor. Reaction system (iii) is used for the FT units, the ZSM-5 hydrocarbon conversion, the MTG reactor, the MTO reactor, and the MOGD reactor. The effluent composition of these units is based on known commercial data or pilot plant data for the units operating at a specified set of conditions (i.e., temperature, pressure, and feed composition). The CBGTL process is designed to ensure that the appropriate conditions are met within the reactor to ensure that the effluent composition that is assumed is valid. Binary decision variables (y) are included within the mathematical model to logically define the existence of specific process units (Eqs. 239-243). That is, if y=0 for a particular unit, then no heat/mass flow will be allowed through the unit and the unit will effectively be removed from the process topology. If y=1 for a unit, then the heat/mass flow through the unit will be governed by the proper operation of the unit.

### Example 3.3

### Fischer-Tropsch Units

The four FT units considered in Examples 1 and 2 utilized either a cobalt or iron catalyst and operated at high or low temperature. The two cobalt based FT units would not facilitate the water-gas-shift reaction and therefore required a minimal level of CO2 input to the units to improve the per-pass conversion of CO. The two iron-based FT units were assumed to facilitate the reverse water-gas-shift reaction and therefore could consume CO₂ within the unit using H₂ to produce the CO necessary for the FT reactions. A key synergy of the reaction conditions in the latter units was the heat needed for the reverse water-gas-shift reaction that is provided by the highly exothermic FT reaction. Though the reverse water-gas-shift reaction is typically unfavorable at the lower operating temperatures of the FT units, the reaction may be indeed facilitated through the use of an appropriate amount of input hydrogen.

The set of possible FT units herein is expanded to consider iron-based systems that will facilitate the forward water-gas-shift reaction within the units. These FT units will require a lower H₂/CO ratio for the FT reaction because steam in the feed will be shifted to H₂ through consumption of CO. These units may be beneficial since certain syngas generation units (e.g., coal gasifiers) will produce a gas that generally has a H2/CO ratio that is much less than the 2/1 requirement for FT synthesis (Baliban et al., 2010; Kreutz et al., 2008, which is incorporated herein by reference as if fully set forth). The downside of the new FT units will be the high quantity of CO₂ that is produced as a result of the water-gas-shift reaction. The framework developed for the CBGTL superstructure will directly examine the benefits and consequences for each of the six FT units to determine which technology produces a refinery with a superior design.

FIG. 32 shows the flowsheet for FT hydrocarbon production within the superstructure. Clean gas from the acid gas removal (AGR) unit is mixed with recycle light gases from a CO₂ separator (CO₂SEP) and split (SP_{CG}) to either the low-wax FT section (SP_{FTM}), the nominalwax FT section (SP_{FTN}), or methanol synthesis (MEOHS). The FT units will operate at a pressure of 20 bar and within the temperature range of 240-320° C. The cobalt-based FT units operate at either low temperature (LTFT; 240° C.) or high temperature (HTFT; 320° C.) and must have a minimal amount of CO₂ in the input stream. Two iron-based FT units will facilitate the reverse water-gas-shift (rWGS) reaction and will operate at low (LTFTRGS; 240° C.) and high temperature (HTFTRGS; 320° C.). The other two iron-based FT units will use the forward reverse water-gas-shift (fWGS) units, operate at a mid-level temperature (267° C.), and produce either minimal (MTFTWGS-M) or nominal (MTFTWGS-N) amounts of wax. The operating conditions of the FT units are summarized in Table 33, below.

TABLE 33
Operating conditions for the process units involved in methanol synthesis
and conversion to liquid hydrocarbon fuels.

| | Temperature | Pressure | |
|---|---|---|---|
| Unit | (° C.) | (bar) | Conv. |
| LT cobalt FT synthesis | 240 | 20 | 80% of CO |
| LT iron FT synthesis | 240 | 20 | 80% of CO |
| MT iron FT synthesis | 267 | 20 | 90% of CO |
| (low wax) | | | |
| MT iron FT synthesis | 267 | 20 | 90% of CO |
| (high wax) | | | |
| HT cobalt FT synthesis | 320 | 20 | 80% of CO |
| HT iron FT synthesis | 320 | 20 | 80% of CO |
| ZSM-5 FT upgrading | 408 | 16 | 100% of |
| | | | hydrocarbons |
| Methanol synthesis | 300 | 50 | 30-40% of CO |
| Methanol-to-gasoline | 400 | 12.8 | 100% of methanol |
| Methanol-to-olefins | 482 | 1.1 | 100% of methanol |
| Olefins-to-gasoline/ | 300 | 50 | 100% of olefins |
| distillate | | | |

Hydrogen may be recycled to any of the FT units to either shift the H₂/CO ratio or the H₂/CO₂ ratio to the appropriate level. Steam may alternatively be used as a feed for the two iron-based fWGS FT units to shift the H₂/CO ratio. CO₂ may be recycled back to the iron-based rWGS FT units to be consumed in the WGS reaction. Similarly, the pressure-swing adsorption (PSA) offgas which will be lean in H₂ may be recycled to the iron-based rWGS FT units for consumption of the CO or CO₂. The effluent from the auto-thermal reactor (ATR) will contain a H₂/CO ratio that is generally above 2/1, and is therefore favorable as a feedstock for FT synthesis (National Academy of Sciences, 2009, which is incorporated herein by reference as if fully set forth). However, the concentration of CO₂ within the ATR effluent will prevent the stream from being fed to the cobalt-based units. The two streams exiting the FT units will be a waxy liquid phase and a vapor phase containing a range of hydrocarbons. The wax will be directed to a hydrocracker (WHC) while the vapor phase is split (SPFTH) for further processing.

Modeling of the four original FT units is described in Examples 1 and 2. The effluent from the two additional FT units (iron-based FT fWGS) is based off of the slurry phase FT units developed by Mobil Research and Development Corporation in the 1980s (Mobil Research & Development Corporation, 1983, 1985, which is incorporated herein by reference as if fully set forth). A H₂/CO ratio of 2/3 is desired for the input feed (Mobil Research & Development Corporation, 1983, 1985, which is incorporated herein by reference as if fully set forth), so a sufficient amount of steam must be added to the feed to promote the forward water-gas-shift reaction. The decomposition of carbon from CO to hydrocarbons and CO₂ is outlined in Table VIII-2 of the minimal-wax FT report (Mobil Research & DevelopmentCorporation, 1983, which is incorporated herein by reference as if fully set forth) and Table VIII-2 of the nominal-wax FT report (Mobil Research & Development Corporation, 1985, which is incorporated herein by reference as if fully set forth), and a 90% conversion of CO in the inlet stream is assumed (Mobil Research & Development Corporation, 1983, 1985, which is incorporated herein by reference as if fully set forth). The syngas species exiting the four iron-based FT reactors will be constrained by water-gasshift equilibrium, as noted in Eq. (216) where (u, u′) is the stream exiting the FT unit u.

N_{u,u′,H}_{₂}_{O}^{S}·N_{u,u′,CO}^{S}=Kᵤ^{WGS}·N_{u,u′,H}_{₂}^{S}·N_{u,u′,CO}_{₂}_{S}∀u∈U_{FT}^{Ir} (216)

The mathematical model will select at most two types of Fischer-Tropsch units to operate in the final process design. This constraint is added because two different kinds of FT units will be able to supply a range of hydrocarbon species that is diverse enough to provide a target composition of liquid products without adding unnecessary complexity to the refinery design (de Klerk, 2011, which is incorporated herein by reference as if fully set forth).

### Example 3.4

### Fischer-Tropsch Product Upgrading

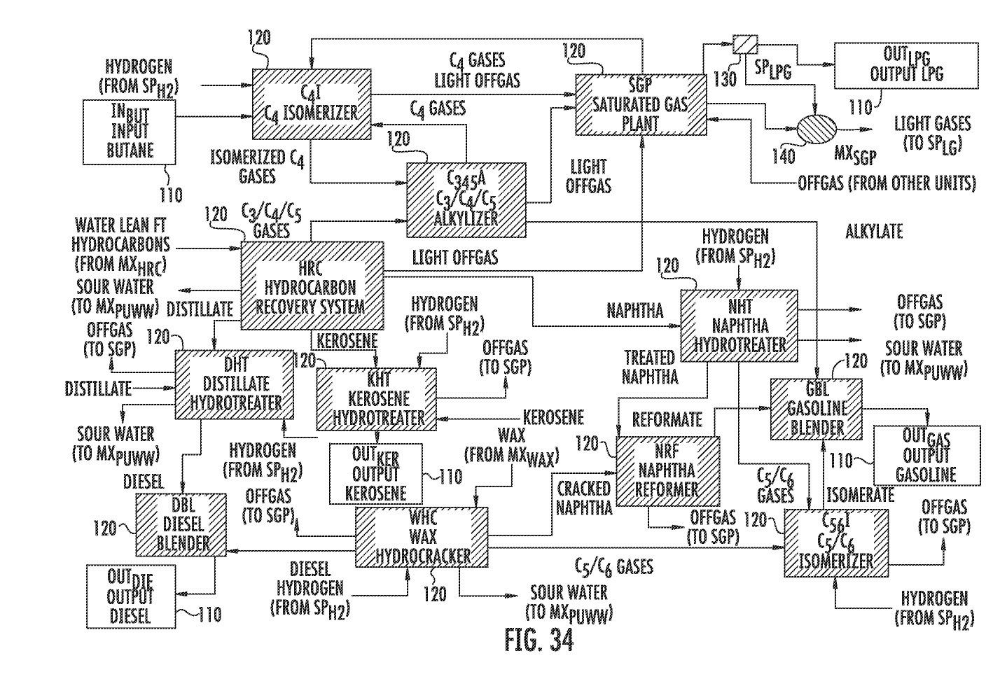

The vapor phase effluent from FT synthesis will contain a mixture of C₁-C₃₀₊ hydrocarbons, water, and some oxygenated species. FIG. 33 details the process flowsheet used to process this effluent stream. The stream will be split (SP_{FTH}) and can pass through a series of treatment units designed to cool the stream and knock out the water and oxygenates for treatment. Initially, the water-soluble oxygenates are stripped (WSOS) from the stream. The stream is then passed to a three-phase separator (VLWS) to remove the aqueous phase from the residual vapor and any hydrocarbon liquid. Any oxygenates that are present in the vapor phase may be removed using an additional separation unit (VSOS). The water lean FT hydrocarbons are then sent to a hydrocarbon recovery column for fractionation and further processing (FIG. 34). The oxygenates and water removed from the stream are mixed (MX_{FTWW}) and sent to the sour stripper mixer (MX_{SS}) for treatment.

The FT hydrocarbons split from SPFTH may also be passed over a ZSM-5 catalytic reactor (FT-ZSM5) operating at 408° C. and 16 bar (Mobil Research & Development Corporation, 1983, which is incorporated herein by reference as if fully set forth) to be converted into mostly gasoline range hydrocarbons and some distillate (Mobil Research & Development Corporation, 1983, 1985, which is incorporated herein by reference as if fully set forth). The ZSM-5 unit will be able to convert the oxygenates to additional hydrocarbons, so no separate processing of the oxygenates will be required for the aqueous effluent. The composition of the effluent from the ZSM-5 unit is shown in Table 43 of the minimal-wax FT reactor Mobil study (Mobil Research & Development Corporation, 1983, which is incorporated herein by reference as if fully set forth) and in Table VIII-3 of the nominal-wax FT reactor Mobil study (Mobil Research & Development Corporation, 1985, which is incorporated herein by reference as if fully set forth). For this study, the ZSM-5 effluent composition is assumed to be equal to the composition outlined in the minimal-wax FT reactor study (Mobil Research & Development Corporation, 1983, which is incorporated herein by reference as if fully set forth). This is modeled mathematically using an atom balance around the ZSM-5 unit and the effluent composition outlined in Table 43 of the Mobil study (Mobil Research & Development Corporation, 1983, which is incorporated herein by reference as if fully set forth). The raw product from FT-ZSM5 is fractionated (ZSM5F) to separate the water and distillate from the gasoline product. The water is mixed with other wastewater knockout (MX_{PUWW}) and the distillate is hydrotreated (DHT) to form a diesel product. The raw ZSM-5 HC product is sent to the LPG-gasoline separation section for further processing (FIG. 36).

The water lean FT hydrocarbons leaving MX_{FTWW} are sent to a hydrocarbon recovery column (HRC), as shown in FIG. 34. The hydrocarbons are split into C₃-C₅ gases, naphtha, kerosene, distillate, wax, offgas, and wastewater (Baliban et al., 2010; Bechtel, 1998, which are incorporated herein by reference as if fully set forth). The upgrading of each stream will follow a detailed Bechtel design (Bechtel, 1992, 1998, which are incorporated herein by reference as if fully set forth) which includes a wax hydrocracker (WHC), a distillate hydrotreater (DHT), a kerosene hydrotreater (KHT), a naphtha hydrotreater (NHT), a naphtha reformer (NRF), a C₄ isomerizer (C₄I), a C₅/C₆ isomerizer (C₅₆I), a C₃/C₄/C₅ alkylation unit (C₃₄₅A), and a saturated gas plant (SGP).

The kerosene and distillate cuts are hydrotreated in (KHT) and (DHT), respectively, to remove sour water and form the products kerosene and diesel. Any additional distillate or kerosene produced in other sections of the refinery will also be directed to these units for processing. The naphtha cut is sent to a hydrotreater (NHT) to remove sour water and separate C₅-C₆ gases from the treated naphtha. The wax cut is sent to a hydrocracker (WHC) where finished diesel product is sent to the diesel blender (DBL) along with the diesel product from (DHT). C5-C6 gases from (NHT) and (WHC) are sent to an isomerizer (C₅₆I). Hydrotreated naphtha is sent to the naphtha reformer (NRF). The C₄ isomerizer (C₄I) converts in-plant and purchased butane to isobutane, which is fed into the alkylation unit (C₃₄₅A). Purchased butane is added to the isomerizer such that 80 wt % of the total flow entering the unit is composed of n-butane. Isomerized C₄ gases are mixed with the C₃-C₅ gases from the (HRC) in (C₃₄₅A), where the C₃-C₅ olefins are converted to highoctane gasoline blending stock. The remaining butane is sent back to (C₄I), while all light gases are mixed with the offgases from other unit and sent to the saturated gas plant (SGP). C₄ gases from (SGP) are recycled back to the (C₄I) and a cut of the C₃ gases are sold as byproduct propane.

### Example 3.5

### Methanol Synthesis and Conversion

The clean gas split (SP_{CG}) from the acid gas recovery unit may be directed to a methanol synthesis unit (MEOHS) for conversion of the syngas to methanol (National Renewable Energy Laboratory, 2011, which is incorporated herein by reference as if fully set forth). The syngas exiting the acid gas recovery unit is heated up to 300° C. prior to entering the MEOHS unit. The MEOHS unit operates at a temperature of 300° C., a pressure of 51 bar, and will assume equilibrium between the water-gas-shift reaction (Eq. (217)) and the methanol synthesis reaction (Eq. (218)) in the effluent stream (MEOHS, u) (National Renewable Energy Laboratory, 2011, which is incorporated herein by reference as if fully set forth).

N_{MEOH,u,H}_{₂}_{O}^{S}·N_{MEOH,u,CO}^{S}=K_{MEOHS}^{WGS}·N_{MEOHS,u,H}_{₂}^{S}·N_{MEOHS,u,CO}_{₂}^{S} (217)

x_{MEOHS,u,CH}_{₃}_{OH}^{S}=K_{MEOHS}^{MSN}·(x_{MEOHS,u,H}_{₂}^{S})²·x_{MEOHS,u,CO}^{S} (218)

Note that the equations for water-gas-shift equilibrium (Eqs. (216) and (217)) utilize molar species flow rates while the methanol synthesis equilibrium (Eq. (218)) and the steam reforming equilibrium (Eqs. (329)-(332)) utilize molar species concentrations. The conservation of total moles across the water-gas-shift equilibrium allows for the use of either species molar flow rates or molar concentrations in the equilibrium reaction without a need for a total molar flow rate variable. The mathematical model was formulated using molar flow rates because the bilinear terms for calculation of the concentration variables are not required for all syngas species. The remainder of the chemical equilibrium equations do not conserve the amount of total moles, so the use of species molar flow rates would require a total molar flow rate variable to balance the equation. In this study, it was found to be computationally beneficial to use species concentration variables to reduce the presence of trilinear or quadrilinear terms that would arise with the use of species molar flow rates. Note that the equilibrium constants used in Eqs. (218) and (329)-(332) have been modified from the values extracted from Aspen to account for the increased pressure of the units.

The “state-of-technology” conditions for methanol synthesis used in this study will require a CO₂ input concentration of 3-8% for methanol synthesis (National Renewable Energy Laboratory, 2011, which is incorporated herein by reference as if fully set forth), though there could exist a potential synergy from a higher CO₂ input concentration (Toyir et al., 2009, which is incorporated herein by reference as if fully set forth). However, an increased level of H₂ may also need to be input to the reactor for consumption via the reverse water-gas-shift reaction. H₂ generated via pressureswing adsorption may not be appropriate if the H₂-lean offgas is primarily used as plant fuel. Alternatively, H₂ provided by electrolysis of water with a non-carbon-based form of electricity (e.g., wind or solar) will have a high capital cost of electrolyzers coupled with a relatively high cost of renewable-based electricity. This may offset the reduction in capital that is achieved if a CO₂ capture technology is not needed for the synthesis gas. The technoeconomical benefits of higher levels of CO₂ input to the methanol synthesis reactor will be the subject of a future investigation. The raw methanol effluent is cooled to 35° C. and sent to a flash unit (MEOH-F) to remove over 95% of the entrained methanol through vapor-liquid equilibrium. The vapor phase is split and mostly recycled (split fraction: 95%) to the methanol synthesis reactor to increase the yield of methanol. The methanol leaving the MEOH-F unit is degassed (MEDEG) via distillation to remove any light vapors. The MEDEG unit is operated as a split unit with a steam utility requirement derived through simulation.

The purified methanol is split (SP_{MEOH}) to either the methanolto-gasoline (MTG) (Mobil Research & Development Corporation, 1978; National Renewable Energy Laboratory, 2011, which are incorporated herein by reference as if fully set forth) process or to the methanol-to-olefins (MTO) and Mobil olefins-togasoline/distillate (MOGD) (Keil, 1999; Tabak et al., 1986; Tabak & Krambeck, 1985; Tabak & Yurchak, 1990, which are incorporated herein by reference as if fully set forth) processes, both of which were developed by Mobil Research and Development in the 1970s and 1980s. More recently, the National Renewable Energy Laboratory performed a full design, simulation, and economic analysis of a biomass-based MTG process (National Renewable Energy Laboratory, 2011, which is incorporated herein by reference as if fully set forth). The MTG process will catalytically convert the methanol to gasoline range hydrocarbons using a ZSM-5 zeolite and a fluidized bed reactor. The MTG effluent is outlined in Table 3.4.2 of the Mobil study (Mobil Research & Development Corporation, 1978, which is incorporated herein by reference as if fully set forth) and in Process Flow Diagram P850-A1402 of the NREL study (National Renewable Energy Laboratory, 2011, which is incorporated herein by reference as if fully set forth). Due to the high level of component detail provided by NREL for both the MTG unit and the subsequent gasoline product separation units, the composition of the MTG reactor used in this study is based on the NREL report. The MTG unit will operate adiabatically at a temperature of 400° C. and 12.8 bar. The methanol feed will be heated to 330° C. and input to the reactor at 14.5 bar. The MTG effluent will contain 44 wt % water and 56 wt % crude hydrocarbons, of which 2 wt % will be light gas, 19 wt % will be C3-C4 gases, and 19 wt % will be C₅₊ gasoline (National Renewable Energy Laboratory, 2011, which is incorporated herein by reference as if fully set forth). The crude hydrocarbons are directed to the LPG-gasoline separation section (FIG. 36), from which 82 wt % will be gasoline, 10 wt % will be LPG, and the balance will be recycle gases. This is modeled mathematically in the process synthesis model by using an atom balance around the MTG unit and assuming a 100% conversion of the methanol entering the MTG reactor (Mobil Research & Development Corporation, 1978; National Renewable Energy Laboratory, 2011, which are incorporated herein by reference as if fully set forth).

Any methanol entering the MTO process unit is heated to 400° C. at 1.2 bar. The MTO fluidized bed reactor operates at a temperature of 482° C. and a pressure of 1.2 bar (Tabak & Yurchak, 1990, which is incorporated herein by reference as if fully set forth). The exothermic heat of reaction within the MTO unit is controlled through generation of low-pressure steam. 100% of the input methanol is converted into olefin effluent containing 1.4 wt % CH₄, 6.5 wt % C₂-C₄ paraffins, 56.4 wt % C₂-C₄ olefins, and 35.7 wt % C₅-C₁₁ gasoline (Tabak & Yurchak, 1990, which is incorporated herein by reference as if fully set forth). The MTO unit is modeled mathematically using an atom balance and a typical composition seen in the literature (Tabak & Yurchak, 1990, which is incorporated herein by reference as if fully set forth). The MTO product is fractionated (MTO-F) to separate the light gases, olefins, and gasoline fractions. The MTO-F unit is a rigorous distillation column that is designed so that approximately 100% of the C₁-C₃ paraffins are recycled back to the refinery, 100% of the C4 paraffins and 100% of the olefins are directed to the MOGD unit, 100% of the gasoline is combined with the remainder of the gasoline generated in the process, and 100% of the water generated in the MTO unit is sent for wastewater treatment. Note that the MTO-F unit is modeled within the process synthesis model as a separator unit with the appropriate utilities (i.e., low-pressure steam and cooling water) that are extracted from simulation of the distillation column.

The separated olefins are sent to the MOGD unit where a fixed bed reactor is used to convert the olefins to gasoline and distillate over a ZSM-5 catalyst. The gasoline/distillate product ratios can range from 0.12 to >100, and the ratio chosen in this study was 0.12 to maximize the production of diesel. The MOGD unit operates at 400° C. and 1 bar and will utilize steam generation to remove the exothermic heat of reaction within the unit. The MOGD unit is modeled with an atom balance and will produce 82% distillate, 15% gasoline, and 3% light gases (Tabak & Yurchak, 1990, which is incorporated herein by reference as if fully set forth). The product will be fractionated (MTODF) to remove diesel and kerosene cuts from the gasoline and light gases. The operational ratio of kerosene to total distillate reported in the literature for the MOGD process is about 30%, though this number may be increased by tailoring the operating conditions within the MTO and MOGD units to yield the appropriate range of hydrocarbons. The MTODF unit will be modeled as a separator unit where 100% of the C₁₁-C₁₃ species are directed to the kerosene cut and 100% of the C14+ species are directed to the diesel cut.

### Example 3.6

### LPG-Gasoline Separation

The gasoline range hydrocarbons produced by the FT-ZSM5 unit, the MTG unit, or the MOGD process must be sent to the LPGgasoline separation flowsheet depicted in FIG. 36. Each hydrocarbon stream is split (SP_{FTZSM}, SP_{MTGHC}, and SP_{MTODHC}, respectively) and sent to a hydrocarbon knockout unit (35° C., 10 bar) for light gas removal via vapor-liquid equilibrium. The first knock-out unit (HCKO1) will not incorporate additional CO₂ separation, so the CO₂ rich light gases recovered from HCKO1 will be recycled back to the process (SP_{LG}). The second knock-out unit (HCKO2) will separate out CO₂ from the recovered light gases via a 1-stage Rectisol unit (CO₂SEP) for sequestration or recycle back to additional process units (MX_{CO2C}). The CO₂ lean light gases will be recycled back to the process.

The crude liquid hydrocarbons recovered from the two knockout units is sent to a deethanizer (DEETH) to remove any C₁-C₂ hydrocarbons. The light HC gases are sent to an absorber column (ABS-COL) where a lean oil recycle is used to strip the C₃₊ HCs from the input. The liquid bottoms from the ABS-COL are then refluxed back to the deethanizer. The C₃₊ HCs from the bottom of the deethanizer are sent to a stabilizer column (STA-COL) where the C₃/C₄ hydrocarbons are removed and alkylated (ALK-UN) to produce iso-octane and an LPG byproduct. Additional iso-butane (INBUT) may be fed to the alkylation unit for increased alkylate production. The bottoms from the stabilizer column is sent to a splitter column (SP-COL) to recover a lean oil recycle from the column top for use in the absorber column. Light and heavy gasoline fractions are recovered from the column top and bottom, respectively. The LPG/alkylate from the alkylation unit is split (LPG-ALK) into an LPG byproduct (OUT_{LPG}) and an alkylate fraction which is blended with the gasoline fractions from the splitter column (OUT_{GAS}). Each of the distillation units is modeled mathematically as a splitter unit where the split fraction of each species to an output stream is given by the information in the Process Flow Diagrams P850-A1501 and P850-A1502 from the NREL study (National Renewable Energy Laboratory, 2011, which is incorporated herein by reference as if fully set forth). All low pressure steam and cooling water needed for each of the units is derived for each of the units in the NREL study. The total amount of process utility that is needed per unit flow rate from the top or bottom of the column is calculated, and this ratio is used as a parameter in the process synthesis model to determine the actual amount of each utility needed based on the unit flow rate. The alkylate was modeled as iso-butane (National Renewable Energy Laboratory, 2011, which is incorporated herein by reference as if fully set forth) and the alkylation unit was modeled using a species balance where the key species, butene, was completely converted to iso-butane. Butene is used as the limiting species in this reaction because it is generally present in a far smaller concentration than iso-butane.

### Example 3.7

### Unit Costs

The total direct costs, TDC, for the CBGTL refinery hydrocarbon production and upgrading units are calculated using estimates from several literature sources (Mobil Research & Development Corporation, 1978, 1983, 1985; National Energy Technology Laboratory, 2007; National Renewable Energy Laboratory, 2011, which are incorporated herein by reference as if fully set forth) using the cost parameters in Table 34 and Eq. (219)

$$T D C = ( 1 + B O P ) \cdot {C}_{o} \cdot \frac{{S}^{sf}}{{S}_{o}} \qquad ( 219 )$$

where Cₒ is the installed unit cost, Sₒ is the base capacity, Sᵣ is the actual capacity, s_{f} is the cost scaling factor, and BOP is the balance of plant (BOP) percentage (site preparation, utility plants, etc.). The BOP is estimated to be 20% of the total installed unit cost. All numbers are converted to 2009 dollars using the GDP inflationindex (US Government Printing Office, 2009, which is incorporated herein by reference as if fully set forth). Detailed cost estimates were not available for the MTO or OGD process units, so the cost associated with these units was estimated from the cost of an atmospheric MTG unit provided by Mobil (Mobil Research & Development Corporation, 1978, which is incorporated herein by reference as if fully set forth). Note that not all units in FIGS. 32-36 are represented in Table 34. Some of the units shown in Table 34 represent the cost of that unit plus any auxillary units needed for proper unit operation. Specifically, (a) the three FT aqueous phase knock-out units are included in the cost of the hydrocarbon recovery column (Bechtel, 1998, which is incorporated herein by reference as if fully set forth), (b) the cost of the FT ZSM-5 fractionator is included in the cost of the FT ZSM-5 unit (Mobil Research & Development Corporation, 1983, 1985, which is incorporated herein by reference as if fully set forth), (c) the MTO fractionator is included in the cost of the MTO unit (National Renewable Energy Laboratory, 2011, which is incorporated herein by reference as if fully set forth), and (d) the OGD fractionator was included in the cost of the OGD unit (Mobil Research & Development Corporation, 1978, which are incorporated herein by reference as if fully set forth).

The total overnight capital, TOC, for each unit is calculated as the sum of the total direct capital, TDC, plus the indirect costs, IC. The IC include engineering, startup, spares, royalties, and contingencies and is estimated to 32% of the TDC. The TOC for each unit must be converted to a levelized cost to compare with the variable feedstock and operational costs for the process. Using the methodology of Kreutz et al. (2008), which is incorporated herein as if fully set forth, the capital charges (CC) for the refinery are calculated by multiplying the levelized capital charge rate (LCCR) and the interest during construction factor (IDCF) by the total overnight capital (Eq. (220)).

CC−LCCR·IDCF·TOC (220)

Kreutz et al. (2008), which is incorporated herein by reference as if fully set forth, calculates an LCCR value of 14.38%/year and IDCF of 1.076. Thus, a multiplier of 15.41%/year is used to convert the overnight capital into a capital charge rate. Assuming an operating capacity (CAP) of 330 days/year and operation/maintenance (OM) costs equal to 5% of the TOC, the total levelized cost (Cost^{U}) associated with a unit is given in Eq. (221).

$${Cost}_{u}^{U} = ( \frac{L C C R \cdot I D C F}{C A P} + \frac{O M}{365} ) \cdot ( \frac{T O {C}_{u}}{Prod \cdot L H {V}_{Prod}} ) \qquad ( 221 )$$

The levelized costs for the units described for hydrocarbon production and upgrading are added to the complete list of CBGTL process units given in Baliban, Elia, and Floudas (2012), which is incorporated herein by reference as if fully set forth.

TABLE 34
CBGTL refinery upgrading unit reference capacities, costs (2009$), and scaling factors

| Description | C₀ (MM$) | S₀ | S_{Max} | Units | Scale basis | sf | Ref. |
|---|---|---|---|---|---|---|---|
| Fischer-Tropsch unit | $12.26 | 23.79 | 60.0 | kg/s | Feed | 0.72 | ^{b, c} |
| Hydrocarbon recovery column | $0.65 | 1.82 | 25.20 | kg/s | Feed | 0.70 | ^{d} |
| Distillate hydrotreater | $2.25 | 0.36 | 81.90 | kg/s | Feed | 0.60 | ^{d} |
| Kerosene hydrotreater | $2.25 | 0.36 | 81.90 | kg/s | Feed | 0.60 | ^{d} |
| Naphtha hydrotreater | $0.68 | 0.26 | 81.90 | kg/s | Feed | 0.65 | ^{d} |
| Wax hydrocracker | $8.42 | 1.13 | 72.45 | kg/s | Feed | 0.55 | ^{d} |
| Naphtha reformer | $4.70 | 0.43 | 94.50 | kg/s | Feed | 0.60 | ^{d} |
| C₅-C₆ isomerizer | $0.86 | 0.15 | 31.50 | kg/s | Feed | 0.62 | ^{d} |
| C₄ isomerizer | $9.50 | 6.21 | — | kg/s | Feed | 0.60 | ^{d} |
| C₃-C₅ alkylation unit | $52.29 | 12.64 | — | kg/s | Feed | 0.60 | ^{d} |
| Saturated gas plant | $7.83 | 4.23 | — | kg/s | Feed | 0.60 | ^{d} |
| FT ZSM-5 reactor | $4.93 | 10.60 | — | kg/s | Feed | 0.65 | ^{b, c} |
| Methanol synthesis | $8.22 | 35.647 | — | kg/s | Feed | 0.65 | ^{e} |
| Methanol degasser | $3.82 | 11.169 | — | kg/s | Feed | 0.70 | ^{e} |
| Methanol-to-gasoline unit | $5.80 | 10.60 | — | kg/s | Feed | 0.65 | ^{a, e} |
| Methanol-to-olefins unit | $3.48 | 10.60 | — | kg/s | Feed | 0.65 | ^{a} |
| Olefins-to-gasoline/diesel unit | $3.48 | 10.60 | — | kg/s | Feed | 0.65 | ^{a} |
| CO₂ separation unit | $5.39 | 8.54 | — | kg/s | Feed | 0.62 | ^{a} |
| Deethanizer | $0.58 | 5.13 | — | kg/s | Feed | 0.68 | ^{a, e} |
| Absorber column | $0.91 | 0.96 | — | kg/s | Feed | 0.68 | ^{a, e} |
| Stabilizer column | $1.03 | 4.57 | — | kg/s | Feed | 0.68 | ^{a, e} |
| Splitter column | $1.01 | 3.96 | — | kg/s | Feed | 0.68 | ^{a, e} |
| HF alkylation unit | $8.99 | 0.61 | — | kg/s | Feed | 0.65 | ^{a, e} |
| LPG/alkylate splitter | $1.06 | 0.61 | — | kg/s | Feed | 0.68 | ^{a, e} |

^{a} Mobil Research and Development Corporation (1978).
^{b} Mobil Research and Development Corporation (1983).
^{c} Mobil Research and Development Corporation (1985).
^{d} Bechtel Corporation (1998).
^{e} National Renewable Energy Laboratory (2011).

### Example 3.8

### Objective Function

The objective function for the model is given in Eq. (222). The summation represents the total cost of liquid fuels production and includes contributions from the feedstocks cost (Cost^{F}), the electricity cost (Cost^{El}), the CO₂ sequestration cost (Cost^{Seq}), and the levelized unit investment cost (Cost^{U}). Each of the terms in Eq. (222) is normalized to the total lower heating value in GJ of products produced. For each case study, the capacity and ratio of liquid fuel products is fixed, so the normalization denominator in Eq. (222) will be a constant parameter. Note that other objective functions (e.g., maximizing the net present value) can be easily incorporated into the model framework.

$$MIN {\sum }_{u ∈ {U}_{In}} {\sum }_{( u , s ) ∈ {S}^{U}} {Cost}_{s}^{F} + {Cost}^{El} + {Cost}^{Seq} + {\sum }_{u ∈ {U}_{Inv}} {Cost}_{u}^{U} \qquad ( 222 )$$

The process synthesis model with simultaneous heat, power, and water integration represents a large-scale non-convex mixedinteger non-linear optimization (MINLP) model that was solved to global optimality using a branch-and-bound global optimization framework that was previously described (Baliban, Elia, Misener, et al., 2012, which is incorporated herein by reference as if fully set forth). The MINLP model contains 32 binary variables, 11,104 continuous variables, 10,103 constraints, and 351 non-convex terms (i.e., 285 bilinear terms, 1 trilinear term, 1 quadrilinear term, and 64 power functions). At each node in the branch-and-bound tree, a mixed-integer linear relaxation of the mathematical model is solved using CPLEX (CPLEX, 2009, which is incorporated herein by reference as if fully set forth) and then the node is branched to create two children nodes. The solution pool feature of CPLEX is utilized during the solution of the relaxed model to generate a set of distinct points (150 for the root node and 10 for all other nodes), each of which is used as a candidate starting point to solve the original model. For each starting point, the current binary variable values are fixed and the resulting NLP is minimized using CONOPT. If the solution to the NLP is less than the current upper bound, then the upper bound is replaced with the NLP solution value. At each step, all nodes that have a lower bound that is within an ∈ tolerance of the current upper bound ((LBnode/UB)≧1−∈) are eliminated from the tree. For a more complete coverage of branch and-bound algorithms, the reader is directed to the textbooks of Floudas (Floudas, 1995, 2000, which are incorporated herein by reference as if fully set forth) and reviews of global optimization methods (Floudas, Akrotirianakis, Caratzoulas, Meyer, & Kallrath, 2005; Floudas & Gounaris, 2009; Floudas & Pardalos, 1995, which are incorporated herein by reference as if fully set forth).

### Example 3.9

### Computational Studies

The proposed process synthesis model was used to analyze twenty-four distinct case studies using perennial biomass (switchgrass), low-volatile bituminous coal (Illinois #6), and natural gas as feedstocks. A global optimization framework was used for each case study, and termination was reached if all nodes in the branch-and-bound tree have been processed or if 100 CPU hours have passed (Baliban, Elia, Misener, et al., 2012, which is incorporated herein by reference as if fully set forth). The ultimate and proximate analysis of the biomass and coal feedstocks and the molar composition of the natural gas feedstock are presented in Examples 3.17-3.23. To examine the effects of potential economies of scale on the final liquid fuels price, three distinct plant capacities were examined to represent a small, medium, or large capacity hybrid energy plant. Based on current petroleum refinery capacities (Energy Information Administration, 2009, which is incorporated herein by reference as if fully set forth), representative sizes of 10 thousand barrels per day (TBD), 50 TBD, and 200 TBD were chosen, respectively. A minimal carbon conversion threshold of 40% was enforced for all of the case studies, and no upper bound was used for the amount of CO₂ that is vented or sequestered. This threshold value was imposed to provide a comparative baseline between all of the case studies, and does not have an effect on the overall process topologies. If no lower threshold value is imposed, then the overall conversion for each study will range between 34% and 39%, which is consistent with the results of a previous study (Baliban, Elia, Misener, et al., 2012, which is incorporated herein by reference as if fully set forth). In general, raising the conversion rate produce more liquid fuels and decrease the byproduct electricity output from the plant, and for a more in-depth analysis, the reader is directed to the previous study (Baliban, Elia, Misener, et al., 2012, which is incorporated herein by reference as if fully set forth). The overall greenhouse gas emission target for each case study is set to have a 50% reduction from petroleum based processes (Baliban, Elia, & Floudas, 2012; Baliban et al., 2011). The current case studies do not include the cost of a carbon tax for any GHG emissions, though the process synthesis framework could be readily extended include a cost for the total lifecycle emissions.

Four superstructure combinations will be investigated to analyze the effect of plant topology on the final liquid fuels cost. These superstructures will consider (1) only Fischer-Tropsch synthesis with fractionation of the vapor effluent, (2) only Fischer-Tropsch synthesis with ZSM-5 catalytic upgrading of the vapor effluent, (3) only methanol synthesis with either the MTG or MOGD process, and (4) a comprehensive superstructure allowing all possibilities from (1), (2), or (3). Note that in superstructures (1), (2), and (4), any wax effluent from the Fischer-Tropsch units will be converted to naphtha and diesel via a wax hydrocracker. Two sets of liquid fuels products (i.e., gasoline/diesel/kerosene and gasoline/diesel) will be considered to determine the effect of these products on the optimal plant topology and overall costs. The ratio of liquid fuel production will be equal to the total 2010 United States demand (Energy Information Administration, 2011, which is incorporated herein by reference as if fully set forth). Note that the process superstructure is also capable of analyzing a variable concentration of output fuels (e.g., max diesel). Each of the 24 case studies discussed below has a label P-CN where P is the type of products produced (GDK—gasoline/diesel/kerosene, GD—gasoline/diesel), C is the plant capacity (S—small, M—medium, L—large), and N is the superstructure number defined above.

The cost parameters (Baliban, Elia, & Floudas, 2012; Baliban et al., 2011, which is incorporated herein by reference as if fully set forth) used for CBGTL process are listed in Table 35. The costs for feedstocks (i.e., coal, biomass, natural gas, freshwater, and butanes) include all costs associated with delivery to the plant gate. The products (i.e., electricity and propane) are assumed to be sold from the plant gate, and do not include the costs expected for transport to the end consumer. The cost of CO₂ capture and compression will be included in the investment cost of the CBGTL refinery while the cost for transportation, storage, and monitoring of the CO₂ is shown in Table 35.

Once the global optimization algorithm has completed, the resulting process topology provides (i) the operating conditions and working fluid flow rates of the heat engines, (ii) the amount of electricity produced by the engines, (iii) the amount of cooling water needed for the engines, and (v) the location of the pinch points denoting the distinct subnetworks. Given this information, the minimum number of heat exchanger matches necessary to meet specifications (i), (ii), (iii), and (iv) are calculated as previously described (Baliban, Elia, & Floudas, 2012; Baliban et al., 2011; Floudas, 1995; Floudas, Ciric, & Grossmann, 1986, which are incorporated herein by reference as if fully set forth). Upon solution of the minimum matches model, the heat exchanger topology with the minimum annualized cost can be found using the superstructure methodology (Elia et al., 2010; Floudas, 1995; Floudas et al., 1986, which are incorporated herein by reference as if fully set forth). The investment cost of the heat exchangers is added to the investment cost calculated within the process synthesis model to obtain the final investment cost for the superstructure.

TABLE 35
Cost parameters (2009$) for the CBGTL refinery.

| Item | Cost | Item | Cost |
|---|---|---|---|
| Coal (LV | $93.41/short ton | Biomass | $139.97/dry |
| bituminous) | ($3/GJ) | (Switchgrass) | metric ton |
| | | | ($8/GJ) |
| Natural gas | $5.39/TSCF¹ ($5.5/GJ) | Freshwater | $0.50/metric ton |
| Butanes | $1.84/gallon | Propanes | $1.78/gallon |
| Electricity | $0.07/kWh | CO₂ TS&M² | $10/metric ton |

¹TSCF—thousand standard cubic feet
²TS&M—transportation, storage, and monitoring

### Example 3.10

### Optimal Process Topologies

The information detailing the optimal process topologies for all case studies is shown in Table 36. Three possible temperature options were used for the biomass gasifier (900° C., 1000° C., 1100° C.), the coal gasifier (1100° C., 1200° C., 1300° C.), the auto-thermal reactor (700° C., 800° C., 950° C.), and the reverse water-gas-shift unit (400° C., 500° C., 600° C.). For all 24 case studies, the biomass and coal solid/vapor fueled gasifiers were utilized in the optimal process design. Thus, each gasifier employed a vapor phase recycle stream as a fuel input along with the solid coal or biomass. Recycle of some of the unreacted synthesis gas to the gasifiers helped to consume some CO₂ generated in the process and reduce the overall process emissions by converting the CO₂ to CO for additional liquid fuels production. For the biomass gasifier, the 900° C. unit is always selected for superstructure 1 and only selected for superstructure 3 at high capacity levels. For all other case studies, the 1100° C. unit is selected. For the coal gasifier, the 1300° C. unit was always selected for superstructures 1, 2, and 3 and the 1100° C. unit was selected for superstructure 4.

TABLE 36
Topological information for the optimal solutions for the 24 case studies.

| Case | GDK- | GDK- | GDK- | GDK- | GDK- | GDK- | GDK- | GDK- | GDK- | GDK- | GDK- | GDK- |
|---|---|---|---|---|---|---|---|---|---|---|---|---|
| Study | S1 | S2 | S3 | S4 | M1 | M2 | M3 | M4 | L1 | L2 | L3 | L4 |
| BGS Temp. (° C.) | 900 | 1100 | 1100 | 1100 | 900 | 1100 | 1100 | 1100 | 900 | 1100 | 900 | 1100 |
| BGS Type | S/V | S/V | S/V | S/V | S/V | S/V | S/V | S/V | S/V | S/V | S/V | S/V |
| CGS Temp. (° C.) | 1300 | 1300 | 1300 | 1100 | 1300 | 1300 | 1300 | 1100 | 1300 | 1300 | 1300 | 1100 |
| CGS Type | S/V | S/V | S/V | S/V | S/V | S/V | S/V | S/V | S/V | S/V | S/V | S/V |
| RGS Temp. (° C.) | — | — | — | 600 | — | — | — | 600 | — | — | — | 600 |
| ATR Temp. (° C.) | 950 | 800 | 800 | 800 | 950 | 950 | 800 | 800 | 950 | 950 | 800 | 950 |
| Min Wax FT | Ir. rWGS | Ir. rWGS | — | — | Ir. rWGS | Ir. rWGS | — | — | Ir. rWGS | Ir. rWGS | — | — |
| Nom. Wax FT | Ir. rWGS | Ir. rWGS | — | Ir. rWGS | Ir. rWGS | Ir. rWGS | — | Ir. rWGS | Ir. rWGS | Ir. rWGS | — | — |
| FT Upgrading | Fract. | ZSM-5 | — | Fract. | Fract. | ZSM-5 | — | Fract. | Fract. | ZSM-5 | — | — |
| MTG Usage | — | — | Y | Y | — | — | Y | Y | — | — | Y | Y |
| MOGD Usage | — | — | Y | Y | — | — | Y | Y | — | — | Y | Y |
| CO2SEQ Usage | Y | Y | Y | Y | Y | Y | Y | Y | Y | Y | Y | Y |
| GT Usage | Y | Y | Y | Y | Y | Y | Y | Y | Y | Y | Y | Y |
| Case | GD- | GD- | GD- | GD- | GD- | GD- | GD- | GD- | GD- | GD- | GD- | GD- |
| Study | S1 | S2 | S3 | S4 | M1 | M2 | M3 | M4 | L1 | L2 | L3 | L4 |
| BGS Temp. (° C.) | 900 | 1100 | 1100 | 110 0 | 900 | 1100 | 1100 | 1100 | 900 | 1100 | 900 | 1100 |
| BGS type | S/V | S/V | S/V | S/V | S/V | S/V | S/V | S/V | S/V | S/V | S/V | S/V |
| CGS Temp. (° C.) | 1300 | 1300 | 1300 | 1100 | 1300 | 1300 | 1300 | 1100 | 1300 | 1300 | 1300 | 1100 |
| CGS type | S/V | S/V | S/V | S/V | S/V | S/V | S/V | S/V | S/V | S/V | S/V | S/V |
| RGS Temp. (° C.) | — | — | — | 600 | — | — | — | 600 | — | — | — | 600 |
| ATR Temp. (° C.) | 950 | 800 | 800 | 800 | 950 | 950 | 800 | 800 | 950 | 950 | 800 | 950 |
| Min wax FT | Ir. rWGS | Ir. rWGS | — | — | Ir. rWGS | Ir. rWGS | — | — | Ir. rWGS | Ir. rWGS | — | — |
| Nom. wax FT | Ir. rWGS | Ir. rWGS | — | Ir. rWGS | Ir. rWGS | Ir. rWGS | — | Ir. rWGS | Ir. rWGS | Ir. rWGS | — | Ir. rWGS |
| FT upgrading | Fract. | ZSM-5 | — | Fract. | Fract. | ZSM-5 | — | Fract. | Fract. | ZSM-5 | — | ZSM-5 |
| MTG usage | — | — | Y | Y | — | — | Y | Y | — | — | Y | Y |
| MOGD usage | — | — | Y | — | — | — | Y | — | — | — | Y | — |
| CO2SEQ usage | Y | Y | Y | Y | Y | Y | Y | Y | Y | Y | Y | Y |
| GT usage | Y | Y | Y | Y | Y | Y | Y | Y | Y | Y | Y | Y |

Specifically listed is the operating temperature of the biomass gasifier (BGS), the coal gasifier (CGS), the auto-thermal reactor (ATR), and the reverse water-gas-shift unit (RGS). The gasifiers are also labeled as either solid/vapor (S/V) or solid (S) fueled, implying the presence or absence of vapor-phase recycle process streams. The presence of a CO2 sequestration system (CO2SEQ) or a gas turbine (GT) is noted using yes (Y) or no (−). The minimum wax and maximum wax Fischer-Tropsch units are designated as either cobalt-based or iron-based units. The iron-based units will either facilitate the forward (fWGS) or reverse water gas-shift (rWGS) reaction. The FT vapor effluent will be upgraded using fractionation into distillate and naphtha (Fract.) or ZSM-5 catalytic conversion. The use of methanol-to-gasoline (MTG) and methanol-to-olefins/olefins-to-gasoline-and-diesel (MTO/MOGD) is noted using yes (Y) or no (−). The results for the complete superstructure and medium sized capacity (M4) are shown in boldface.

Selection of the gasifier operating temperatures in the optimal topology represents a balance between (i) the levels of oxidant input to the gasifier, (ii) the extent of consumption of CO₂ via the reverse water-gas-shift reaction, and (iii) the level of waste heat generated from syngas cooling. Lower gasifier temperatures will have less favorable conditions for CO₂ consumption due to lower values of the water-gas-shift equilibrium constant and a smaller amount of waste heat for use in steam generation and ultimately electricity production. However, lower temperatures will require lower levels of O₂ for combustion within the gasifier which reduces the investment and utility cost for oxygen generation and may increase the overall efficiency of the gasifier. The alternative disadvantages with a higher O₂ in the higher temperature gasifiers are balanced by an increase in the CO₂ reduction potential and the additional waste-heat generated. The operating temperature selected in the 24 case studies reflects the trade-offs between emissions reduction, electricity production, and overall process efficiency for the entire refinery.

The auto-thermal reformer temperature was selected to be 950° C. for twelve of the case studies and 800° C. for the remaining twelve studies (see Table 36). A 950° C. unit is always used for superstructure 1, used for superstructure 2 in the medium and large plants, and used in superstructure 4 for the large plants. Selection of the temperature for the auto-thermal reformer will have similar topological effects as the gasifiers, though the overall conversion of CH₄ will also increase with increasing temperature. The use of the highest temperature reformer is beneficial since approximately 90% of the input CH₄ can be converted to syngas using a H₂O/CH₄ ratio of approximately 1.2-1.5. Ultimately, this will also decrease the working capacity of the FT synthesis or methanol synthesis units because the input CH₄ is an inert species that will not be separated until downstream of these units. The selection of the 800° C. units for the remaining studies generally converts 82-85% of the CH₄, though the decrease in the oxygen requirement to the unit provides an economic benefit to the decreased conversion of the natural gas.

A dedicated reverse water-gas-shift unit was not selected for either product composition and plant capacity that used superstructures 1, 2, or 3. For each of these case studies, the proper syngas ratio requirements for the FT and methanol synthesis was met via light gas recycle to either the gasifiers or the auto-thermal reactor units. For the case studies using superstructure 4, a 600° C. reverse water-gas-shift unit was utilized to both consume CO₂ generated in the process and shift the syngas ratios for conversion. All of the case studies generated H₂ using pressure-swing adsorption and O₂ using air separation. The H₂ was utilized mostly for product upgrading and for injection, with the balance being sent to the reverse water-gas-shift units to consume some CO₂. Note that H₂ separation is required for hydrotreating and hydrocracking within the product upgrading section. Electrolyzers were not utilized in any case study due to the high capital ($500/kW) and electricity costs of the unit. The electricity input to the electrolyzers is assumed to come from a non-carbon based source (e.g., wind/solar), which was assumed to have a high cost (i.e., $0.10/kWh). Note that input electricity from a carbon-based source (i.e., biomass/coal/natural gas) is not considered because the process superstructure accounts for H₂ generation from pressure-swing adsorption. A decrease in the non-carbon based electricity cost may have an effect on the electrolyzer use, as noted in a previous study (Baliban et al., 2011, which is incorporated herein by reference as if fully set forth). Both a gas and steam turbine are used in each case study to produce electricity for the process and to partially sell as a byproduct. To reduce the GHG emissions from the processes, each case study utilized CO₂ capture and sequestration both upstream of synthesis gas conversion and downstream of the gas turbine engine.

The case studies using superstructures 1 and 2 required FT synthesis of the hydrocarbons, and each case study utilized an iron-based catalyst within both the minimal-wax and nominalwax reactors. Additionally, the reverse water-gas-shift reaction was facilitated in most of the case studies, with the exception of the minimal-wax reactor in superstructure 2 for the medium and large capacities. In the former case studies, the iron-based units can take advantage of the exothermic FT reaction to provide heat for the endothermic reverse water-gas-shift reaction (Baliban, Elia, & Floudas, 2012; Baliban et al., 2011, which is incorporated herein by reference as if fully set forth). In the latter studies, the additional CO₂ that is generated from the FT reactors is captured and recycled back to the process to minimize the GHG emissions. Due to the constraints of the process superstructure, upgrading of the vapor phase FT effluent utilized a fractionation scheme for superstructure 1 and the ZSM-5 catalyst for superstructure 2. For superstructure 3, the syngas was converted to methanol rather than hydrocarbons via the FT reaction. For all case studies using this superstructure, both the methanol-to-gasoline and methanolto-olefins/distillate processes are utilized to produce the liquid fuels in the appropriate output ratios. In the case studies using superstructure 4, the technologies used for liquid fuels production are highly dependent on the plant capacity and the type of fuels produced. For the six studies with superstructure 4, the minimalwax FT unit was never utilized and the methanol-to-gasoline process was always utilized. The nominal-wax iron-based rWGS FT unit was used for the two small plants, the two medium plants, and the large plant that does not produce kerosene. For the five case studies that used FT, the vapor phase was always converted to gasoline-range hydrocarbons using ZSM-5. The MOGD process was used to generate diesel and kerosene for all plant sizes in the GDK case studies. In the GD case studies, the MOGD process was not utilized and all diesel was generated from wax hydrocracking.

The results for the complete superstructure and medium sized capacity (M4) are shown in boldface in Table 36. For each of these cases, both the biomass and coal gasifiers were solid/vapor fueled units operating at 1100° C. A dedicated reverse water-gas-shift unit operating at 600° C. is used and the auto-thermal reactor operates at 800° C. for both studies. The liquid fuels are produced via (i) catalytic ZSM-5 upgrading of the iron-based rWGS FT effluent, (ii) wax hydrocracking, and (iii) methanol-to-gasoline for both studies and by MOGD for the study requiring kerosene production.

### Example 3.11

### Overall Costs of Liquid Fuels

The overall cost of liquid fuel production (in $/GJ) is based on the costs of feedstocks, capital investment, operation and maintenance (O&M), and CO₂ sequestration and can be partially defrayed using byproduct sales of LPG and electricity. Feedstock costs are based on the as-delivered price for (i) the three major carbon feedstocks (coal, biomass, and natural gas), (ii) butanes needed for the isomerization process (Baliban et al., 2010, 2011; Bechtel, 1992, which is incorporated herein by reference as if fully set forth), and (iii) freshwater needed to make-up for process losses (Baliban et al., 2012a, which is incorporated herein by reference as if fully set forth). Table 37 outlines the breakdown of the cost contribution for each case study, as well as the lower bound and the optimality gap values. The total cost is also converted into a break-even oil price (BEOP) in $/barrel based on the refiner's margin for gasoline, diesel, or kerosene (Baliban et al., 2011; Kreutz et al., 2008, which is incorporated herein by reference as if fully set forth), and represents the price of crude oil at which the CBGTL process becomes economically competitive with petroleum based processes.

The overall cost values range between $17.33 and $18.79/GJ for a small plant, $16.06$17.66/GJ for a medium plant, and $14.76$16.20/GJ for a large plant. For a medium sized plant producing gasoline, diesel, and kerosene, the optimization model for the complete superstructure (i.e., case study GDK-M4) selects a topology with an overall cost of $16.25/GJ or $79.83/bbl crude oil equivalent. The upper bound value found at the termination of the global optimization algorithm is 4.56% above the lower bound value of $15.51/GJ. When only gasoline and diesel are produced in the general medium sized plant (GD-M4), the overall cost of liquid fuel production for a medium sized plant with the most general superstructure is $16.06/GJ or $78.74/bbl crude oil equivalent with a 5.35% optimality gap from its lower bound value of $15.20/GJ. Negative values in the cost contributions from electricity and propane represent the profit gained from selling these items as byproducts. In all of the 24 case studies, the selected plant topologies are net producers of electricity and propane (see Table 37, below).

TABLE 37
Overall cost results for the 24 case studies.

| Contribution | Case study | | | | | | | | | | | |
|---|---|---|---|---|---|---|---|---|---|---|---|---|
| to cost ($/GJ | GDK- | GDK- | GDK- | GDK- | GDK- | GDK- | GDK- | GDK- | GDK- | GDK- | GDK- | GDK- |
| of products) | S1 | S2 | S3 | S4 | M1 | M2 | M3 | M4 | L1 | L2 | L3 | L4 |
| Coal | 3.15 | 3.18 | 3.20 | 2.91 | 3.32 | 3.31 | 3.05 | 3.16 | 3.21 | 3.08 | 3.33 | 3.37 |
| Biomass | 2.71 | 2.69 | 2.70 | 2.74 | 2.75 | 2.81 | 2.73 | 2.70 | 2.69 | 2.73 | 2.69 | 2.67 |
| Natural gas | 3.58 | 3.48 | 3.43 | 4.14 | 3.08 | 3.02 | 3.80 | 3.53 | 3.40 | 3.78 | 3.14 | 3.02 |
| Butane | 0.28 | 0.31 | 0.40 | 0.28 | 0.29 | 0.25 | 0.34 | 0.33 | 0.36 | 0.25 | 0.34 | 0.36 |
| Water | 0.03 | 0.02 | 0.03 | 0.02 | 0.02 | 0.02 | 0.02 | 0.02 | 0.02 | 0.02 | 0.02 | 0.02 |
| CO2 Seq. | 0.51 | 0.50 | 0.51 | 0.50 | 0.50 | 0.49 | 0.51 | 0.50 | 0.51 | 0.51 | 0.51 | 0.51 |
| Investment | 11.15 | 10.81 | 10.22 | 10.03 | 8.29 | 8.16 | 7.50 | 7.65 | 7.25 | 7.44 | 6.64 | 6.70 |
| O&M | 3.27 | 3.17 | 3.00 | 2.94 | 2.43 | 2.40 | 2.20 | 2.25 | 2.13 | 2.18 | 1.95 | 1.97 |
| Electricity | −5.69 | −5.43 | −5.26 | −5.96 | −2.86 | −2.82 | −3.34 | −3.72 | −3.20 | −3.92 | −3.02 | −3.48 |
| Propane | −0.19 | −0.15 | −0.20 | −0.15 | −0.17 | −0.14 | −0.20 | −0.17 | −0.17 | −0.21 | −0.22 | −0.19 |
| Total ($/GJ) | 18.79 | 18.59 | 18.02 | 17.46 | 17.66 | 17.51 | 16.61 | 16.25 | 16.20 | 15.86 | 15.37 | 14.95 |
| Total ($/bbl) | 94.32 | 93.18 | 89.90 | 86.72 | 87.85 | 87.00 | 81.85 | 79.83 | 79.52 | 77.58 | 74.84 | 72.40 |
| Lower bound | 17.73 | 17.86 | 17.31 | 16.92 | 16.68 | 16.54 | 15.83 | 15.51 | 15.35 | 15.32 | 14.74 | 14.40 |
| ($/GJ) | | | | | | | | | | | | |
| Gap | 5.63% | 3.92% | 3.92% | 3.10% | 5.52% | 5.55% | 4.67% | 4.56% | 5.24% | 3.40% | 4.16% | 3.67% |
| Contribution | Case study | | | | | | | | | | | |
| to cost ($/GJ | GD- | GD- | GD- | GD- | GD- | GD- | GD- | GD- | GD- | GD- | GD- | GD- |
| of products) | S1 | S2 | S3 | S4 | M1 | M2 | M3 | M4 | L1 | L2 | L3 | L4 |
| Coal | 2.71 | 3.38 | 2.75 | 2.72 | 3.25 | 3.13 | 3.34 | 3.23 | 3.19 | 3.39 | 3.27 | 3.27 |
| Biomass | 2.75 | 2.65 | 2.74 | 2.75 | 2.68 | 2.68 | 2.66 | 2.66 | 2.68 | 2.65 | 2.66 | 2.65 |
| Natural gas | 4.62 | 2.98 | 4.51 | 4.59 | 3.30 | 3.56 | 3.07 | 3.30 | 3.42 | 2.95 | 3.24 | 3.21 |
| Butane | 0.26 | 0.26 | 0.31 | 0.32 | 0.30 | 0.38 | 0.33 | 0.36 | 0.33 | 0.27 | 0.29 | 0.33 |
| Water | 0.03 | 0.03 | 0.02 | 0.02 | 0.02 | 0.03 | 0.02 | 0.02 | 0.02 | 0.02 | 0.03 | 0.03 |
| CO2 Seq. | 0.50 | 0.50 | 0.50 | 0.50 | 0.50 | 0.50 | 0.50 | 0.50 | 0.50 | 0.50 | 0.50 | 0.50 |
| Investment | 11.28 | 11.09 | 10.22 | 10.07 | 8.11 | 8.38 | 7.33 | 7.48 | 6.85 | 6.99 | 6.33 | 6.52 |
| O&M | 3.31 | 3.26 | 3.00 | 2.96 | 2.38 | 2.46 | 2.15 | 2.20 | 2.01 | 2.05 | 1.86 | 1.91 |
| Electricity | −6.53 | −5.72 | −5.91 | −6.43 | −3.01 | −3.65 | −2.84 | −3.56 | −2.83 | −2.81 | −2.73 | −3.53 |
| Propane | −0.16 | −0.20 | −0.16 | −0.16 | −0.21 | −0.17 | −0.20 | −0.14 | −0.18 | −0.20 | −0.14 | −0.14 |
| Total ($/GJ) | 18.59 | 18.06 | 17.99 | 17.33 | 17.33 | 17.30 | 16.36 | 16.06 | 16.01 | 15.84 | 15.30 | 14.76 |
| Total ($/bbl) | 93.16 | 90.14 | 89.73 | 85.99 | 85.99 | 85.82 | 80.46 | 78.74 | 78.45 | 77.47 | 74.42 | 71.31 |
| Lower bound | 17.77 | 17.28 | 17.17 | 16.41 | 16.52 | 16.40 | 15.86 | 15.20 | 15.10 | 14.99 | 14.79 | 13.97 |
| ($/GJ) | | | | | | | | | | | | |
| Gap | 4.39% | 4.30% | 4.55% | 5.32% | 4.67% | 5.20% | 3.07% | 5.35% | 5.67% | 5.34% | 3.33% | 5.33% |

The case studies where the plant topologies produce gasoline, diesel, and kerosene are labeled as GDK, and the topologies that produce gasoline and diesel are labeled as GD. The small (S), medium (M), and large (L) case studies are each labeled with the superstructure number, where (1) indicates that only Fischer Tropsch synthesis with fractionation of the vapor effluent is considered, (2) only Fischer-Tropsch synthesis with ZSM-5 catalytic upgrading of the vapor effluent, (3) only methanol synthesis with either the MTG or MOGD process, and (4) a comprehensive superstructure allowing all possibilities from (1), (2), or (3). The contribution to the total costs (in $/GJ) come from coal, biomass, natural gas, butanes, water, CO₂ sequestration (CO₂. Seq.), and the investment. Propane is always sold as a byproduct while electricity may be sold as a byproduct (negative value). The overall costs are reported in ($/GJ) and ($/bbl) basis, along with the lower bound values in ($/GJ) and the optimality gap between the reported solution and the lower bound. The results for the complete superstructure and medium sized capacity (M4) are shown in boldface.

For a given capacity level, Table 37 shows that the lowest overall cost is achieved through the use of the most general superstructure topology. Additionally, the second lowest cost is consistently found with superstructure 3, suggesting that the methanol synthesis/conversion process units generally yield a plant design with a lower overall cost. However, the decrease in cost between superstructure 3 (only methanol) and superstructure 4 (methanol/FT) implies that there is a degree of synergy that can be achieved through the use of both technologies. The resulting level of synergy is likely to be tied to the capacity of the plant and the composition of liquid fuels that will be produced. The CBGTL case studies using superstructures 2 (FT with ZSM-5 upgrading) have a lower cost ultimately due to a decrease in the complexity of the FT synthesis and upgrading section of the plant. In some case studies (i.e., GDK-L2, GD-L2, and GD-M2), the investment cost of the plant with ZSM-5 upgrading was higher than that for the corresponding case study without ZSM-5 upgrading. The increase in investment is due to a higher overall flow rate of syngas through the refinery due to (i) increased recycle flow of the unreacted syngas to decrease feedstock costs or (2) increased flow of the feedstocks to produce additional byproduct electricity.

### Example 3.12

### Parametric Analysis

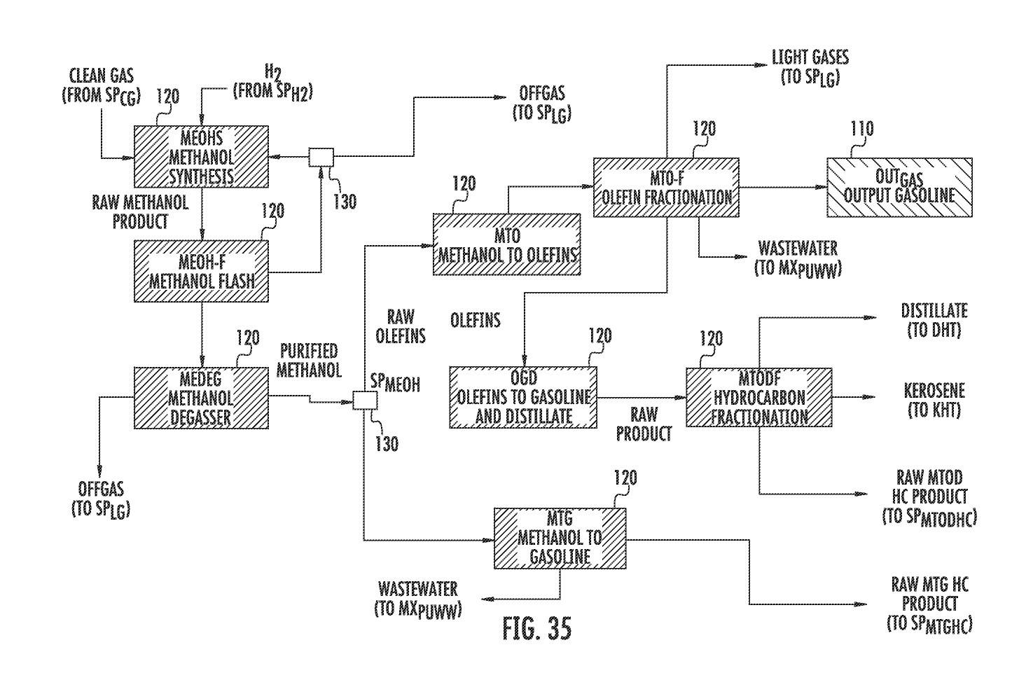

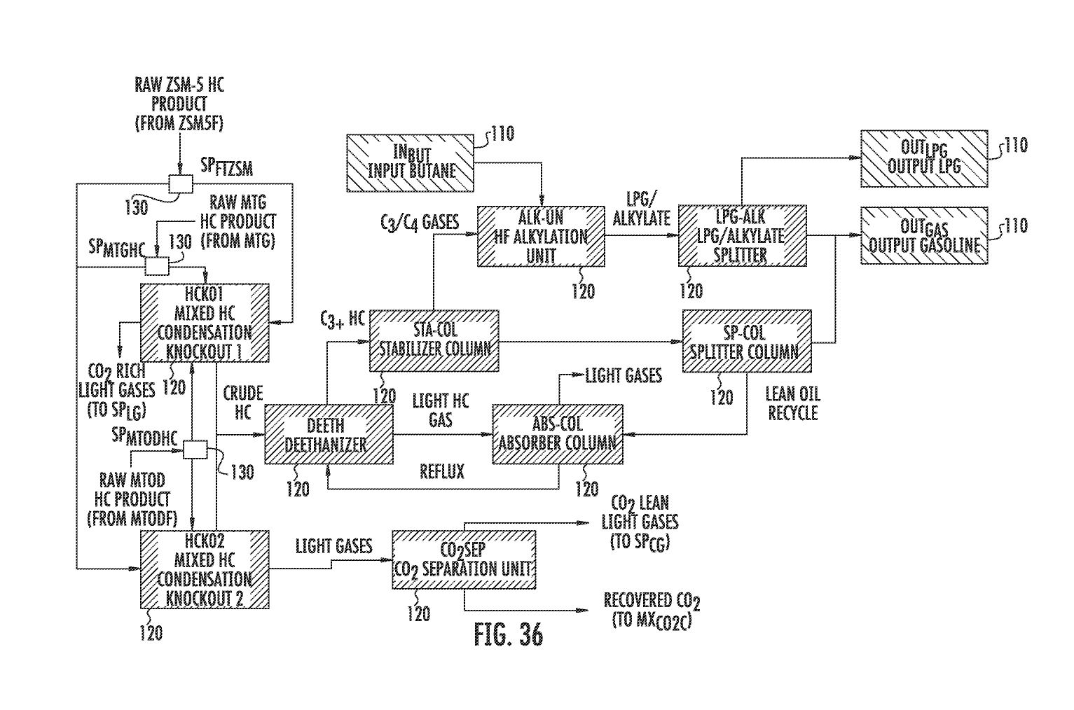

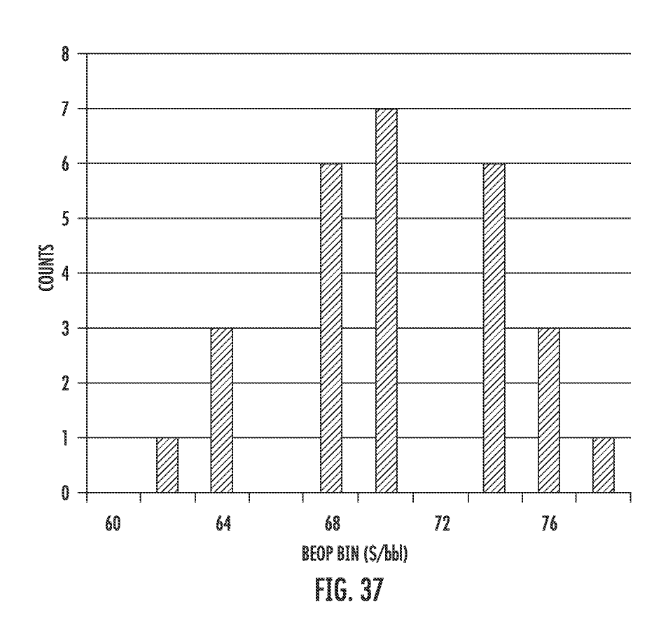

Table 37 indicates that the largest contribution to the overall fuels cost is associated with the capital investment (i.e., capital charges and operation/maintenance). A reduction in total plant cost may be achieved through innovation of novel technologies rather than relying on economies of scale for more mature processes (Adams & Barton, 2011, which is incorporated herein by reference as if fully set forth). However, the coal, biomass, and natural gas may have a wide variability in the overall cost of liquid fuel production. Depending on the demand for these materials and the plant location throughout the country, the feedstock costs may be higher or lower than the national average. Given the delivered feedstock costs in Table 35 and the feedstock lower heating values in Table 45, the cost per unit energy is calculated for coal ($3.0/GJ), biomass ($8.0/GJ), and natural gas ($5.5/GJ). These cost parameters represent conservative estimates (Energy Information Administration, 2011; Kreutz et al., 2008; Larson et al., 2009; National Academy of Sciences, 2009, which are incorporated herein by reference as if fully set forth) for the total delivered cost of a particular feedstock, and it is important to investigate how the BEOP will be affected if these cost parameters are reduced. As an illustrative example, the BEOP for case study GDK-M4 is calculated assuming either low, nominal, or high cost values for each of the three feedstocks. These respective values are (i) $2/GJ, $2.5/GJ, and $3/GJ for coal, (ii) $5/GJ, $6.5/GJ, and $8/GJ for biomass, and (iii) $4/GJ, $4.75/GJ, and $5.5/GJ for natural gas. The BEOP was calculated for each of the 27 parameter combinations, and the histogram of results is shown in FIG. 37.

Each cost bin in FIG. 37 represents a $2/barrel window for the BEOP. That is, the first bin represents all of the parameter combinations that had a BEOP between $60/bbl and $62/bbl, the second bin is between $62/bbl and $64/bbl, and so on. The histogram shows a Gaussian-like distribution with two major peaks in the $68/bbl-$72/bbl range with a total of 13 counts. The shape of the histogram can be inferred from Table 37 since the contribution of each feedstock to the overall cost is relatively similar. The singular peak in the leftmost bin corresponds to a BEOP of $62.7/bbl and is obtained if the low parameters are used for each feed. The highest BEOP is equal to $80.0/bbl, and is obtained if all of the high parameter values are used.

### Example 3.13

### Investment Costs

The plant investment cost is further decomposed into cost contributions from different sections of the plant in Table 38, namely the syngas generation, syngas cleaning, hydrocarbon production, hydrocarbon upgrading, hydrogen/oxygen production, heat and power integration, and wastewater treatment sections. The syngas generation section is consistently the highest contributing factor in the investment cost due to the capital intensive coal and biomass gasifier units. The next highest contributing factors are the syngas cleaning, hydrogen/oxygen production, and heat and power integration sections, followed by the hydrocarbon production section, and finally the hydrocarbon upgrading and wastewater treatment sections.

The total investment cost ranges from $1166 to $1296 MM for small plants producing gasoline, diesel, and kerosene, $4359-$4823 MM for medium plants, and $15,446-$17,309 MM for large plants. The normalized investment costs, however, reveal the economies of scale obtained in larger sized plants, ranging from $116 k to $130 k/bpd for small plants, $87 k-$96 k/bpd for medium plants, and $78 k-$87 k/bpd for large plants. Among the small plant case studies, the case with the most general superstructure (i.e., GDK-S4) is able to achieve the lowest investment cost. For larger sized plants, however, GDK-M3 and GDK-L3 case studies have the lowest investment costs for medium and large plants case studies, respectively. Conversely, the case studies using superstructure 1 from all capacity levels have the highest total investment cost.

Comparisons between the GDK and GD case studies reveal interesting trade-offs in investment costs. For the small plants case studies, plant topologies that produce only gasoline and diesel result in higher investment costs than the ones that produce gasoline, diesel, and kerosene. The increased cost of the small GD case studies is due to a higher flow rate of syngas throughout the process units due to a slightly higher level of recycle than the GDK small case studies. The increased investment costs for the small GD studies do lead into smaller levels of feedstock usage than the small GDK studies, and therefore have a lower overall cost of liquid fuels production (see Table 37). For the medium and large GD case studies, the topologies that produce gasoline and diesel fuels consistently yield lower total investment costs than their GDK counterparts due to the less complicated refining that is needed to produce kerosene.

TABLE 38
Breakdown of the investment costs for the 24 case studies.

| | Case study | | | | | | | | | | | |
|---|---|---|---|---|---|---|---|---|---|---|---|---|
| Contribution | GDK- | GDK- | GDK- | GDK- | GDK- | GDK- | GDK- | GDK- | GDK- | GDK- | GDK- | GDK- |
| to cost (MM$) | S1 | S2 | S3 | S4 | M1 | M2 | M3 | M4 | L1 | L2 | L3 | L4 |
| Syngas generation | 494 | 492 | 476 | 478 | 1422 | 1443 | 1314 | 1369 | 5362 | 5702 | 5063 | 5187 |
| Syngas cleaning | 240 | 234 | 222 | 225 | 813 | 786 | 769 | 758 | 3153 | 3186 | 2932 | 2870 |
| Hydrocarbon production | 218 | 208 | 170 | 166 | 738 | 731 | 566 | 603 | 2682 | 2775 | 1985 | 2148 |
| Hydrocarbon upgrading | 24 | 22 | 16 | 16 | 165 | 147 | 96 | 100 | 343 | 326 | 207 | 206 |
| Hydrogen/oygen production | 145 | 138 | 139 | 126 | 789 | 770 | 768 | 754 | 2424 | 2495 | 2588 | 2451 |
| Heat and power integration | 146 | 137 | 138 | 129 | 781 | 742 | 747 | 767 | 2521 | 2412 | 2364 | 2405 |
| Wastewater treatment | 29 | 26 | 28 | 26 | 115 | 127 | 99 | 98 | 377 | 412 | 307 | 305 |
| Total (MM$) | 1296 | 1258 | 1188 | 1166 | 4823 | 4745 | 4359 | 4450 | 16,862 | 17,309 | 15,446 | 15,572 |
| Total ($/bpd) | 129,647 | 125,754 | 118,809 | 116,609 | 96,451 | 94,897 | 87,177 | 88,993 | 87,211 | 86,547 | 79,335 | 77,858 |
| | Case study | | | | | | | | | | | |
| Contribution | GD- | GD- | GD- | GD- | GD- | GD- | GD- | GD- | GD- | GD- | GD- | GD- |
| to cost (MM$) | S1 | S2 | S3 | S4 | M1 | M2 | M3 | M4 | L1 | L2 | L3 | L4 |
| Syngas generation | 500 | 511 | 486 | 483 | 1373 | 1480 | 1270 | 1318 | 5068 | 5183 | 4875 | 4868 |
| Syngas cleaning | 244 | 242 | 219 | 223 | 785 | 805 | 779 | 764 | 3071 | 3035 | 2770 | 2919 |
| Hydrocarbon production | 218 | 209 | 161 | 166 | 735 | 785 | 554 | 594 | 2399 | 2602 | 1867 | 2190 |
| Hydrocarbon upgrading | 25 | 22 | 16 | 15 | 154 | 150 | 90 | 86 | 338 | 327 | 190 | 205 |
| Hydrogen/oygen production | 148 | 139 | 134 | 131 | 784 | 756 | 742 | 734 | 2365 | 2413 | 2471 | 2292 |
| Heat and power integration | 146 | 140 | 143 | 126 | 767 | 773 | 733 | 759 | 2326 | 2304 | 2237 | 2381 |
| Wastewater treatment | 30 | 27 | 30 | 27 | 120 | 123 | 94 | 94 | 367 | 401 | 314 | 298 |
| Total (MM$) | 1311 | 1290 | 1189 | 1171 | 4717 | 4872 | 4261 | 4348 | 15,935 | 16,265 | 14,723 | 15,153 |
| Total ($/bpd) | 131,118 | 128,975 | 118,893 | 117,083 | 94,335 | 97,434 | 85,226 | 86,958 | 79,677 | 81,326 | 73,615 | 75,764 |

The major sections of the plant include the syngas generation section, syngas cleaning, hydrocarbon production, hydrocarbon upgrading, hydrogen/oxygen production, heat and power integration, and wastewater treatment blocks. The values are reported in MM$ and normalized with the amount of fuels produced ($/bpd). The results for the complete superstructure and medium sized capacity (M4) are shown in boldface.

### Example 3.14

### Material and Energy Balances

The overall material and energy balances for the 24 case studies are shown in Tables 39 and 40, respectively. The biomass and coal flow rates are based of dry tons (dt) while the natural gas is shown in million standard cubic feet (mscf). From Tables 38 and 39, it can be seen that coal provides the most energy input to the plant, followed generally by natural gas, and then biomass. For example, the most small capacity plant with the most general superstructure (GDK-S4) requires 69.56 dt/h coal, 51.08 dt/h for biomass, and 1.83 mscf/h natural gas. These values correspond to 596 MW energy input from coal, 224 MW from biomass, and 497 MW from natural gas. This distribution remains relatively consistent when the plant size increases. For the medium sized plant (case study GDK-M4), 377.39 dt/h is needed for coal, 251.95 dt/h for biomass, and 7.77 mscf/h, corresponding to 3234 MW energy input for coal, 1106 MW for biomass, and 2114 MW for natural gas. Case study GDK-L4 requires 1607.23 dt/h coal, 997.60 dt/h biomass, and 26.64 mscf/h natural gas, corresponding to 13,775 MW energy input from coal, 4377 MW from biomass, and 7250 MW from natural gas. The smaller contribution of biomass relative to the other two feedstocks is due to the higher $/GJ costs associated with biomass. The highest driving force for the use of biomass is the lifecycle GHG reduction potential, but the use of CO₂ sequestration from the 24 case studies (see Table 39) will reduce the biomass requirement for the plant. A restriction on the amount of CO₂ that is captured for sequestration (e.g., no nearby available locations for CO₂ storage) will ultimately increase the biomass feedstock requirement, and the biomass could become the largest energy contributor to the refinery. The authors note that the biomass requirement for the large case studies (i.e., 200,000/bpd) is necessary to achieve a life-cycle GHG emissions that is 50% lower than petroleum-based processes. Though the biomass-based plant designs by the National Renewable Energy Laboratory use approximately 2000 dry tons/day (National Renewable Energy Laboratory, 2011; Spath et al., 2005, which are incorporated herein by reference as if fully set forth), the availability of biomass may be substantially higher in several counties (e.g., Midwestern United States) after land-use change or an increase in crop yields (Department of Energy, 2005, which is incorporated herein by reference as if fully set forth).

TABLE 39
Overall material balance for the 24 case studies.

| | Case study | | | | | | | | | | | |
|---|---|---|---|---|---|---|---|---|---|---|---|---|
| Material | GDK- | GDK- | GDK- | GDK- | GDK- | GDK- | GDK- | GDK- | GDK- | GDK- | GDK- | GDK- |
| balances | S1 | S2 | S3 | S4 | M1 | M2 | M3 | M4 | L1 | L2 | L3 | L4 |
| Biomass (dt/h) | 50.56 | 50.28 | 50.42 | 51.08 | 256.34 | 262.08 | 254.39 | 251.95 | 1005.02 | 1018.08 | 1002.86 | 997.60 |
| Coal (dt/h) | 75.16 | 75.95 | 76.31 | 69.56 | 396.13 | 395.36 | 364.37 | 377.39 | 1532.36 | 1468.83 | 1588.67 | 1607.23 |
| Natural gas (mscf/h) | 1.58 | 1.53 | 1.51 | 1.83 | 6.78 | 6.66 | 8.38 | 7.77 | 29.98 | 33.28 | 27.65 | 26.64 |
| Butane (kBD) | 0.21 | 0.23 | 0.30 | 0.21 | 1.07 | 0.92 | 1.26 | 1.21 | 5.28 | 3.71 | 5.00 | 5.38 |
| Water (kBD) | 18.18 | 14.34 | 18.85 | 16.01 | 80.05 | 77.55 | 75.88 | 68.38 | 296.85 | 333.54 | 306.86 | 313.53 |
| Gasoline (kBD) | 6.72 | 6.72 | 6.72 | 6.72 | 33.60 | 33.60 | 33.60 | 33.60 | 134.39 | 134.39 | 134.39 | 134.39 |
| Diesel (kBD) | 2.15 | 2.15 | 2.15 | 2.15 | 10.77 | 10.77 | 10.77 | 10.77 | 43.10 | 43.10 | 43.10 | 43.10 |
| Kerosene (kBD) | 1.13 | 1.13 | 1.13 | 1.13 | 5.63 | 5.63 | 5.63 | 5.63 | 22.51 | 22.51 | 22.51 | 22.51 |
| LPG (kBD) | 0.14 | 0.11 | 0.15 | 0.11 | 0.63 | 0.51 | 0.74 | 0.62 | 2.53 | 3.14 | 3.25 | 2.85 |
| Seq. CO₂ (tonne/h) | 240.04 | 239.65 | 240.14 | 239.61 | 1183.81 | 1167.69 | 1200.70 | 1198.94 | 4796.55 | 4805.53 | 4807.09 | 4801.23 |
| Vented CO₂ (tonne/h) | 0.00 | 0.00 | 0.00 | 0.00 | 15.23 | 29.69 | 0.00 | 0.00 | 0.00 | 0.00 | 0.00 | 0.00 |
| Electricity (MW) | −193.14 | −184.16 | −178.57 | −202.29 | −484.88 | −477.54 | −567.06 | −631.42 | −2171.37 | −2661.25 | −2045.52 | −2357.99 |
| Material | Case study | | | | | | | | | | | |
| balances | GD-S1 | GD-S2 | GD-S3 | GD-S4 | GD-M1 | GD-M2 | GD-M3 | GD-M4 | GD-L1 | GD-L2 | GD-L3 | GD-L4 |
| Biomass (dt/h) | 51.33 | 49.47 | 51.21 | 51.32 | 249.75 | 250.28 | 248.04 | 248.07 | 998.77 | 988.91 | 991.27 | 990.18 |
| Coal (dt/h) | 64.64 | 80.78 | 65.60 | 64.81 | 387.91 | 373.28 | 398.69 | 385.57 | 1523.11 | 1619.62 | 1558.90 | 1562.09 |
| Natural gas (mscf/h) | 2.04 | 1.31 | 1.99 | 2.02 | 7.27 | 7.84 | 6.75 | 7.27 | 30.17 | 26.03 | 28.55 | 28.28 |
| Butane (kBD) | 0.19 | 0.20 | 0.23 | 0.24 | 1.11 | 1.42 | 1.24 | 1.35 | 4.90 | 4.05 | 4.33 | 4.95 |
| Water (kBD) | 20.51 | 19.18 | 12.51 | 14.84 | 79.22 | 92.56 | 68.38 | 79.22 | 286.85 | 350.22 | 366.90 | 360.23 |
| Gasoline (kBD) | 7.57 | 7.57 | 7.57 | 7.57 | 37.86 | 37.86 | 37.86 | 37.86 | 151.44 | 151.44 | 151.44 | 151.44 |
| Diesel (kBD) | 2.43 | 2.43 | 2.43 | 2.43 | 12.14 | 12.14 | 12.14 | 12.14 | 48.56 | 48.56 | 48.56 | 48.56 |
| LPG (kBD) | 0.12 | 0.15 | 0.12 | 0.12 | 0.79 | 0.61 | 0.75 | 0.51 | 2.61 | 2.90 | 2.13 | 2.08 |
| Seq. CO₂ (tonne/h) | 238.72 | 239.15 | 238.76 | 238.80 | 1196.62 | 1194.09 | 1196.04 | 1192.62 | 4778.68 | 4782.98 | 4771.66 | 4770.88 |
| Vented CO₂ (tonne/h) | 0.00 | 0.00 | 0.00 | 0.00 | 0.00 | 0.00 | 0.00 | 0.00 | 0.00 | 0.00 | 0.00 | 0.00 |
| Electricity (MW) | −221.38 | −193.96 | −200.46 | −218.06 | −509.61 | −619.46 | −481.53 | −602.94 | −1916.43 | −1908.04 | −1849.71 | −2393.96 |

The inputs to the CBGTL process are biomass, coal, natural gas, butane, and water, while the outputs include gasoline, diesel, kerosene, LPG, sequestered and vented CO₂, and electricity. Biomass and coal are input in dry metric tons per hour (dt/h), natural gas in million standard cubic feet per hour (mscf/h), liquids in thousand barrels per day (kBD), and CO₂ in metric tons per hour (tonne/h). The results for the complete superstructure and medium sized capacity (M4) are shown in boldface.

TABLE 40
Overall energy balance for the 24 case studies.

| Energy | Case study | | | | | | | | | | | |
|---|---|---|---|---|---|---|---|---|---|---|---|---|
| balacnes | GDK- | GDK- | GDK- | GDK- | GDK- | GDK- | GDK- | GDK- | GDK- | GDK- | GDK- | GDK- |
| (MW) | S1 | S2 | S3 | S4 | M1 | M2 | M3 | M4 | L1 | L2 | L3 | L4 |
| Biomass | 222 | 221 | 221 | 224 | 1125 | 1150 | 1116 | 1106 | 4410 | 4467 | 4401 | 4377 |
| Coal | 644 | 651 | 654 | 596 | 3395 | 3388 | 3123 | 3234 | 13,133 | 12,589 | 13,616 | 13,775 |
| Natural gas | 429 | 417 | 411 | 497 | 1845 | 1812 | 2279 | 2114 | 8157 | 9057 | 7522 | 7250 |
| Butane | 13 | 14 | 18 | 13 | 65 | 56 | 77 | 74 | 321 | 226 | 304 | 327 |
| Gasoline | 428 | 428 | 428 | 428 | 2141 | 2141 | 2141 | 2141 | 8563 | 8563 | 8563 | 8563 |
| Diesel | 153 | 153 | 153 | 153 | 766 | 766 | 766 | 766 | 3065 | 3065 | 3065 | 3065 |
| Kerosene | 78 | 78 | 78 | 78 | 390 | 390 | 390 | 390 | 1558 | 1558 | 1558 | 1558 |
| LPG | 9 | 7 | 9 | 7 | 38 | 31 | 45 | 38 | 154 | 191 | 197 | 173 |
| Electricity | 193 | 184 | 179 | 202 | 485 | 478 | 567 | 631 | 2171 | 2661 | 2046 | 2358 |
| Efficiency | 65.8% | 65.3% | 64.9% | 65.3% | 59.4% | 59.4% | 59.3 | 60.7% | 59.6% | 60.9% | 59.7% | 61.1% |
| (%) | | | | | | | | | | | | |
| Energy | Case study | | | | | | | | | | | |
| balacnes | GD- | GD- | GD- | GD- | GD- | GD- | GD- | GD- | GD- | GD- | GD- | GD- |
| (MW) | S1 | S2 | S3 | S4 | M1 | M2 | M3 | M4 | L1 | L2 | L3 | L4 |
| Biomass | 225 | 217 | 225 | 225 | 1096 | 1098 | 1088 | 1089 | 4383 | 4339 | 4350 | 4345 |
| Coal | 554 | 692 | 562 | 555 | 3325 | 3199 | 3417 | 3305 | 13,054 | 13,881 | 13,361 | 13,388 |
| Natural gas | 554 | 357 | 541 | 550 | 1979 | 2133 | 1837 | 1979 | 8210 | 7082 | 7768 | 7694 |
| Butane | 12 | 12 | 14 | 14 | 67 | 86 | 75 | 82 | 298 | 246 | 263 | 301 |
| Gasoline | 482 | 482 | 482 | 482 | 2412 | 2412 | 2412 | 2414 | 9649 | 9649 | 9649 | 9649 |
| Diesel | 173 | 173 | 173 | 173 | 863 | 863 | 863 | 863 | 3454 | 3454 | 3454 | 3454 |
| LPG | 7 | 9 | 7 | 7 | 48 | 37 | 45 | 31 | 159 | 176 | 129 | 126 |
| Electricity | 221 | 194 | 200 | 218 | 510 | 619 | 482 | 603 | 1916 | 1908 | 1850 | 2394 |
| Efficiency | 65.7% | 67.1% | 64.3% | 65.5% | 59.3% | 60.3% | 59.3 | 60.6% | 58.5% | 59.4% | 58.6% | 60.7% |
| (%) | | | | | | | | | | | | |

The energy inputs to the CBGTL process come from biomass, coal, natural gas, and butane, and the energy outputs are gasoline, diesel, kerosene, LPG, and electricity. The energy efficiency of the process is calculated by dividing the total energy output with the total energy inputs to the process.

Almost all of the case studies do not vent CO₂ from the process, and utilize CO₂ sequestration to reduce the lifecycle GHG emissions of the plant. The GDK-M1 and GDK-M2 studies vent a small amount of CO₂, though the CO₂ is only 1-2% of the total CO₂ produced by the plant. The balance of the CO₂ is captured for sequestration. The high utilization of CO₂ sequestration allows for an increased use of the cheaper fossil fuels coal and natural gas, which can be anywhere from $3/GJ to $6/GJ less expensive than biomass. The biomass does provide negative emission values from CO₂ intake from the atmosphere during cultivation and additional soil storage from land use change, so a level of biomass input on a mass/energy basis that is roughly equivalent to that of coal or natural gas is still required. The electricity production ranges from 179 to 221 MW for small plants, 478-631 MW for medium plants, and 1850-2661 MW for large plants. In all case studies, a high amount of electricity is produced to help lower the overall cost of fuels for the plant. The electricity output also improves the efficiency of the topologies, with GD-S2, GDK-S1, and GD-S1 achieving the highest energy efficiencies (i.e., 67.1%, 65.8%, and 65.7%, respectively) compared to other case studies in their subcategories (see Table 9). The energy efficiency values are calculated by dividing the total energy output (i.e., fuel products, propane, or electricity) by the total energy input (i.e., via coal, biomass, natural gas, butane, or electricity). If electricity is output from the system, the value is listed as negative in Table 39 and the magnitude of the energy value in Table 40 is added to the total output. If the value is positive in Table 39, then this energy is added to the total input to the system. The overall energy efficiency of the CBGTL topologies producing gasoline, diesel, and kerosene ranges between 58.5 and 67.1% for all plant sizes.

### Example 3.15

### Carbon and Greenhouse Gas Balances

The overall carbon balance for the CBGTL processes is shown in Table 41 and highlights the eight major points where carbon is either input or output from the system. The results for the complete superstructure and medium sized capacity (M4) case studies are highlighted in the table using boldface. Carbon that is input to the system via air is neglected due to the low flow rate relative to the other eight points. Over 99% of the input carbon is supplied from the coal, biomass, and natural gas while the balance is supplied by the butane input to the isomerization and alkylation units. The trends seen in feedstock use from Table 39 are consistently displayed in the input carbon flow rates in Table 41. That is, for all of the case studies, a majority of the carbon is input from coal and CO₂ sequestration is highly utilized to reduce the GHG emissions. The biomass and natural gas provide roughly equivalent amounts of input carbon to the refineries, which combined represent approximately 40% of the input carbon. The output amount of carbon in the total product is constant for each plant capacity, which is consistent with the constant production capacity that is required for each feedstockconversion rate. The amount of carbon leaving as LPG is around 1% of that leaving as gasoline, kerosene, and diesel. For all of the case studies, most of the CO₂ generated from the process is captured and sequestered, with little or no CO₂ venting.

For each of the case studies, the carbon conversion rate was set as a lower bound (i.e., 40%) for the mathematical model. Thus, the conversion of carbon in the four feedstocks to any of the four liquid products must be at least as large as the set conversion rate. All of the 24 case studies reached this bound, implying that this constraint was active in the optimal solution. Note that this constraint can be relaxed if a smaller conversion rate of liquid fuels is desired. Ultimately, this will have the effect of decreasing the overall fuels cost by potentially generating additional byproduct electricity. However, recent studies have suggested that the CBGTL process designs will tend to convert between 34% and 37% of the feedstock carbon when a lower conversion threshold of 25% is set (Baliban, Elia, Misener, et al., 2012, which is incorporated herein by reference as if fully set forth). Therefore, the minimum threshold of 40% will serve to provide a baseline measure of comparison between the case studies while not dramatically impacting the final overall cost.

TABLE 41
Carbon balances (in kg/s) for the optimal solutions for the 24 case studies.

| Case | GDK- | GDK- | GDK- | GDK- | GDK- | GDK- | GDK- | GDK- | GDK- | GDK- | GDK- | GDK- |
|---|---|---|---|---|---|---|---|---|---|---|---|---|
| study | S1 | S2 | S3 | S4 | M1 | M2 | M3 | M4 | L1 | L2 | L3 | L4 |
| Biomass | 5.90 | 5.87 | 5.88 | 5.96 | 29.91 | 30.58 | 29.68 | 29.39 | 117.25 | 118.78 | 117.00 | 116.39 |
| Coal | 16.91 | 17.09 | 17.17 | 15.65 | 89.13 | 88.96 | 81.98 | 84.91 | 344.78 | 330.49 | 357.45 | 361.63 |
| Natural gas | 7.23 | 7.12 | 7.02 | 8.47 | 31.47 | 30.91 | 38.88 | 36.06 | 139.13 | 154.47 | 128.30 | 123.65 |
| Butane | 0.19 | 0.21 | 0.27 | 0.19 | 0.98 | 0.84 | 1.15 | 1.11 | 4.83 | 3.39 | 4.57 | 4.92 |
| Gasoline | 7.78 | 7.78 | 7.78 | 7.78 | 38.91 | 38.91 | 38.91 | 38.91 | 155.64 | 155.64 | 155.64 | 155.64 |
| Diesel | 2.85 | 2.85 | 2.85 | 2.85 | 14.24 | 14.24 | 14.24 | 14.24 | 56.95 | 56.95 | 56.95 | 56.95 |
| Kerosene | 1.40 | 1.40 | 1.40 | 1.40 | 6.98 | 6.98 | 6.98 | 6.98 | 27.93 | 27.93 | 27.93 | 27.93 |
| LPG | 0.10 | 0.08 | 0.11 | 0.08 | 0.46 | 0.38 | 0.55 | 0.46 | 1.87 | 2.33 | 2.41 | 2.11 |
| Vented CO₂ | 0.00 | 0.00 | 0.00 | 0.00 | 1.15 | 2.25 | 0.00 | 0.00 | 0.00 | 0.00 | 0.00 | 0.00 |
| Seq. CO₂ | 18.20 | 18.17 | 18.20 | 18.16 | 89.74 | 88.51 | 91.02 | 90.88 | 363.60 | 364.28 | 364.39 | 363.95 |
| % | 40.0% | 40.0% | 40.0% | 40.0% | 40.0% | 40.0% | 40.0 | 40.0% | 40.0% | 40.0% | 40.0% | 40.0% |
| Conversion | | | | | | | | | | | | |
| Case | GD- | GD- | GD- | GD- | GD- | GD- | GD- | GD- | GD- | GD- | GD- | GD- |
| study | S1 | S2 | S3 | S4 | M1 | M2 | M3 | M4 | L1 | L2 | L3 | L4 |
| Biomass | 5.99 | 5.77 | 5.97 | 5.99 | 29.14 | 29.20 | 28.94 | 28.94 | 116.52 | 115.37 | 115.65 | 115.52 |
| Coal | 14.54 | 18.18 | 14.76 | 14.58 | 87.28 | 83.99 | 89.70 | 86.75 | 342.70 | 364.41 | 350.75 | 351.47 |
| Natural gas | 9.45 | 6.09 | 9.22 | 9.38 | 33.75 | 36.38 | 31.33 | 33.75 | 140.03 | 120.79 | 132.49 | 131.23 |
| Butane | 0.18 | 0.18 | 0.21 | 0.22 | 1.01 | 1.29 | 1.13 | 1.23 | 4.48 | 3.70 | 3.96 | 4.52 |
| Gasoline | 8.77 | 8.77 | 8.77 | 8.77 | 43.85 | 43.85 | 43.85 | 43.85 | 175.39 | 175.39 | 175.39 | 175.39 |
| Diesel | 3.21 | 3.21 | 3.21 | 3.21 | 16.04 | 16.04 | 16.04 | 16.04 | 64.18 | 64.18 | 64.18 | 64.18 |
| LPG | 0.09 | 0.11 | 0.09 | 0.09 | 0.58 | 0.45 | 0.55 | 0.38 | 1.93 | 2.15 | 1.58 | 1.54 |
| Vented CO₂ | 0.00 | 0.00 | 0.00 | 0.00 | 0.00 | 0.00 | 0.00 | 0.00 | 0.00 | 0.00 | 0.00 | 0.00 |
| Seq. CO₂ | 18.10 | 18.13 | 18.10 | 18.10 | 90.71 | 90.52 | 90.66 | 90.40 | 362.24 | 362.57 | 361.71 | 361.65 |
| % | 40.0% | 40.0% | 40.0% | 40.0% | 40.0% | 40.0% | 40.0 | 40.0% | 40.0% | 40.0% | 40.0% | 40.0% |
| Conversion | | | | | | | | | | | | |

Carbon is input to the process via coal, biomass, natural gas, or butanes and exits the process as liquid product, LPG byproduct, vented CO₂, or sequestered (Seq.) CO₂. The small amount of CO₂ input to the system in the purified oxygen stream (<0.01%) is neglected. The results for the complete superstructure and medium sized capacity (M4) are shown in boldface.

The greenhouse gas (GHG) emission balances for the case studies are shown in Table 42. For each of the studies, the total GHG emission target was set to be equal to 50% of the emissions from a standard petroleum based process. For a typical emission level of 500 kg of CO₂ equivalent per barrel, this implies that the total well-to-wheel GHG emissions for the CBGTL refinery must be less than 250 kg CO₂eq/bbl. The GHG emission rates (in kg CO₂eq/s) for the ten major point sources in the refinery are listed in Table 42 and include (a) acquisition and transportation of the biomass, coal, natural gas, and butane feeds, (b) transportation and use of the gasoline, diesel, kerosene, and LPG, (c) transportation and sequestration of any CO₂, and (d) venting of any process emissions. The GHG emissions for feedstock acquisition and transportation in (a), product transportation in (b), and CO₂ transportation in (c) are calculated from the GREET model for well-to-wheel emissions (Argonne National Laboratory. GREET 1.8b, 2007, which is incorporated herein by reference as if fully set forth) and assuming transportation distances for feedstocks (50 miles), products (100 miles), and CO2 (50 miles). The GHG emissions from product use in (b) are calculated assuming that each product will be completely combustion to generate CO₂ that is simply vented to the atmosphere.

From Table 42, it is clear that a major component of the lifecycle emissions are attributed to the liquid fuels. In fact, over 80% of the liquid fuel emissions result from combustion of these fuels in light and heavy duty vehicles. The total emissions from transportation of the feedstocks, products, and CO₂ represents the balanced of the lifecycle emissions for the process. To balance the GHG lifecycle, the CO₂ removed from the atmosphere due to storage in the biomass or storage in the soil is included in the total emissions for biomass.

Note that while the net emissions for biomass is negative, there will still be a positive component to the emissions for biomass harvesting and transportation. It is important to observe that though the coal was the highest energy input to the refinery, the emissions contribution from natural gas is higher from coal or biomass.

TABLE 42
Greenhouse gas (GHG) balances for the optimal solutions for the 24 case studies.

| Case | GDK- | GDK- | GDK- | GDK- | GDK- | GDK- | GDK- | GDK- | GDK- | GDK- | GDK- | GDK- |
|---|---|---|---|---|---|---|---|---|---|---|---|---|
| study | S1 | S2 | S3 | S4 | M1 | M2 | M3 | M4 | L1 | L2 | L3 | L4 |
| Biomass | −27.76 | −27.60 | −27.68 | −28.04 | −140.73 | −143.88 | −139.66 | −138.32 | −551.76 | −558.93 | −550.58 | −547.68 |
| Coal | 2.11 | 2.13 | 2.14 | 1.95 | 11.10 | 11.08 | 10.21 | 10.58 | 42.94 | 41.16 | 44.52 | 45.04 |
| Natural gas | 3.42 | 3.32 | 3.27 | 3.95 | 14.67 | 14.41 | 18.13 | 16.81 | 64.87 | 72.02 | 59.82 | 57.65 |
| Butane | 0.02 | 0.03 | 0.03 | 0.02 | 0.12 | 0.10 | 0.14 | 0.13 | 0.58 | 0.41 | 0.55 | 0.60 |
| Gasoline | 30.73 | 30.73 | 30.73 | 30.73 | 153.64 | 153.64 | 153.64 | 153.64 | 614.54 | 614.54 | 614.54 | 614.54 |
| Diesel | 11.17 | 11.17 | 11.17 | 11.17 | 55.86 | 55.86 | 55.86 | 55.86 | 223.45 | 223.45 | 223.45 | 223.45 |
| Kerosene | 5.49 | 5.49 | 5.49 | 5.49 | 27.46 | 27.46 | 27.46 | 27.46 | 109.83 | 109.83 | 109.83 | 109.83 |
| LPG | 0.43 | 0.35 | 0.45 | 0.34 | 1.89 | 1.55 | 2.23 | 1.87 | 7.63 | 9.48 | 9.80 | 8.60 |
| Vented CO₂ | 0.00 | 0.00 | 0.00 | 0.00 | 4.23 | 8.25 | 0.00 | 0.00 | 0.00 | 0.00 | 0.00 | 0.00 |
| Seq. CO₂ | 3.33 | 3.33 | 3.34 | 3.33 | 16.44 | 16.22 | 16.68 | 16.65 | 66.62 | 66.74 | 66.77 | 66.68 |
| Total GHG | 250.00 | 250.00 | 250.00 | 250.00 | 250.00 | 250.00 | 250.00 | 250.00 | 250.00 | 250.00 | 250.00 | 250.00 |
| (kg/bbl) | | | | | | | | | | | | |
| Case | GD- | GD- | GD- | GD- | GD- | GD- | GD- | GD- | GD- | GD- | GD- | GD- |
| study | S1 | S2 | S3 | S4 | M1 | M2 | M3 | M4 | L1 | L2 | L3 | L4 |
| Biomass | −28.18 | −27.16 | −28.11 | −28.18 | −137.11 | −137.40 | −136.17 | −136.19 | −548.33 | −542.91 | −547.68 | −543.61 |
| Coal | 1.81 | 2.26 | 1.84 | 1.82 | 10.87 | 10.46 | 11.17 | 10.80 | 42.68 | 45.38 | 45.04 | 43.77 |
| Natural gas | 4.41 | 2.84 | 4.30 | 4.37 | 15.74 | 16.96 | 14.61 | 15.74 | 65.29 | 56.32 | 57.65 | 61.19 |
| Butane | 0.02 | 0.02 | 0.03 | 0.03 | 0.12 | 0.16 | 0.14 | 0.15 | 0.54 | 0.45 | 0.55 | 0.55 |
| Gasoline | 34.62 | 34.62 | 34.62 | 34.62 | 173.12 | 173.12 | 173.12 | 173.12 | 692.49 | 692.49 | 614.54 | 692.49 |
| Diesel | 12.59 | 12.59 | 12.59 | 12.59 | 62.95 | 62.95 | 62.95 | 62.95 | 251.79 | 251.79 | 223.45 | 251.79 |
| LPG | 0.35 | 0.44 | 0.36 | 0.37 | 2.37 | 1.85 | 2.25 | 1.55 | 7.87 | 8.76 | 109.83 | 6.27 |
| Vented CO₂ | 0.00 | 0.00 | 0.00 | 0.00 | 0.00 | 0.00 | 0.00 | 0.00 | 0.00 | 0.00 | 6.27 | 0.00 |
| Seq. CO₂ | 3.32 | 3.32 | 3.32 | 3.32 | 16.62 | 16.58 | 16.61 | 16.56 | 66.37 | 66.43 | 0.00 | 66.26 |
| Total GHG | 250.00 | 250.00 | 250.00 | 250.00 | 250.00 | 250.00 | 250.00 | 250.00 | 250.00 | 250.00 | 66.68 | 250.00 |
| (kg/bbl) | | | | | | | | | | | | |

The total GHG emissions (in CO₂ equivalents−kg CO₂ eq/s) for feedstock acquisition and transportation, product transportation and use, CO₂ sequestration, and process venting are shown for each study. Process feedstocks include biomass, coal, natural gas, and butane while products include gasoline, diesel, kerosene, and LPG. The results for the complete superstructure and medium sized capacity (M4) are shown in boldface.

This example has detailed the development of a framework for the process synthesis of a thermochemical hybrid coal, biomass, and natural gas to liquids plant that incorporates multiple possibilities for hydrocarbon production and hydrocarbon upgrading. The framework also included a simultaneous heat, power, and water integration to compare the costs of utility generation and wastewater treatment in the overall cost of liquid fuels. This example expands on the CBGTL process in Examples 1 and 2 by directly quantifying the economic and environmental benefits that are associated with (i) Fischer-Tropsch synthesis and subsequent hydrocarbon upgrading and (ii) methanol synthesis, conversion to hydrocarbons, and subsequent upgrading. The proposed optimization model was tested using 24 distinct case studies that are derived from two combinations of products, three plant capacities, and four superstructure possibilities. The overall conversion of carbon from feedstock to liquid products was selected to be 40% and the greenhouse gas reduction target was equal to 50% of current petroleum based refineries. Each case study was globally optimized using a branch-and-bound global optimization algorithm to theoretically guarantee that the cost associated with the optimal design was within 3-6% of the best value possible.

When producing gasoline, diesel, and kerosene in ratios commensurate with Untied States demands, the overall cost of liquid fuels production ranges from $86/bbl to $94/bbl for small plants (10,000 barrels per day; kBD), $79/bbl-$88/bbl for medium plants (50 kBD), and $72/bbl-$80/bbl for large plants (200 kBD). When only gasoline and diesel are produced in a ratio consistent with national demand, the cost decreases for each of the capacities to a range of $85/bbl-$93/bbl for small, $78/bbl-$86/bbl for medium, and $71/bbl-$78/bbl for large plants. This decrease in cost is generally due to the reduction in investment needed to fractionate and convert the distillate to diesel only opposed to both diesel and kerosene. For the four different superstructure possibilities investigated in this study, it is evident that FT synthesis followed by fractionation (superstructure 1) and upgrading is more expensive than FT synthesis followed by catalytic ZSM-5 conversion to gasoline-range hydrocarbons (superstructure 2). Additionally, methanol synthesis, conversion to hydrocarbons, and subsequent upgrading (superstructure 3) is consistently cheaper than FT synthesis for all capacity levels. This is due to the decrease in investment cost associated with hydrocarbon production and upgrading when compared to FT synthesis. These findings indicate that the methanol route is preferential to the FT route when following an “either or” logic. However, investigation of a “combination” superstructure that considered all of the topologies (superstructure 4) in superstructures 1-3 indicates that a combination of FT synthesis and methanol synthesis will provide the lowest overall cost. In this case, the MTG route provides a majority of the gasoline while a majority of the distillate (diesel and kerosene) is generated through fractionation and refining of the FT effluent. Though over 80% of the final hydrocarbons were produced via the methanol synthesis route, the final process topologies show that the ability to consume CO₂ in iron-based FT reactors helps to reduce feedstock costs and therefore provide an economic advantage over a topology that utilizes only methanol synthesis.

### Example 3.16

### Mathematical Model for Process Synthesis with Simultaneous Heat, Power, and Water Integration

The nomenclature for all terms in the mathematical model for process synthesis with simultaneous heat, power, and water integration is shown below. All constraints included in the model are listed subsequently with a corresponding description of how that particular equation governs proper operation of the process design.

Process Units

The set of units, U, is presented in full detail in Table 43 and defined formally in Eq. (223). Note that several units in Table 43 are listed as uₙ. The n subscript represents the consideration of multiple forms of the same process unit, each with a distinct set of operating conditions (e.g., temperature and pressure). Though these unit properties are generally given as continuous variables in a process synthesis problem, they have been assumed to take discrete choices and will be modeled using binary variables.

u∈U={Complete set of process units listed in Table 43} (223)

Process Species

The set of all species, S, is listed in Table 44 and defined formally in Eq. (224).

s∈S={Complete set of species listed in Table 44} (224)

Indices/Sets

The indices are used throughout the mathematical model are listed below.

u: Process unit index
s: Species index
a: Atom index
p: Proximate analysis index
r: Reaction index
i: General counting index

The set, U, is defined as the complete set of process units. Several subsets of units are then defined for specific areas of the CBGTL process as presented below.

u_{BGS}={u:u=BGSn}
u_{CGS}={u:u=CGSn}
U_{RGS}={u:u=RGSn}
u_{ATR}={u:u=ATRn}

The set of all atoms, A, includes C, H, O, N, S, Cl, Ar, and a generic Ash atom. Typically, the biomass and coal ash will consist of multiple metal oxides, but the ash is assumed to be inert in the CBGTL process, so the treatment of the ash as an atomic element is justified.

a∈A={C,H,O,N,S,Cl,Ar,Ash}

The list of all unit connections, UC, is derived below.

UC={(u,u′):∃a connection between unit u and unit u′ in the superstructure}

Using a priori knowledge about the operations of each unit in the CBGTL process, the complete set of species that can possibly exist in a stream from unit u to unit u′ is defined as S_{u,u′}^{UC}. The set (u,u′, s) ∈=S_{UF} is then constructed from all streams in UC along with the set of all species s that exist within a given unit u (S^{U}).

S^{UF}={(u,u′,s):∃s∈S_{u,u′}^{UC}}

S^{U}={(s,u):∃(u,u′,s)∈S^{UF} or ∃(u′,u,s)∈S^{UF}}

Parameters

With the exception of all biomass and coal species, char, and the pseudocomponents, the molecular formula is equal to the species index defined in Table 44. The pseudocomponent hydrocarbons and oxygenate formulas are given by Bechtel while the formulas for biomass and coal compounds are derived from the ultimate analysis and normalized to one mole of carbon. Char has been assumed to consist completely of carbon and ash has been assigned a generic molecular weight of 1.0 g/mol. The atomic ratio (AR_{s,a}) of atom a in species s is derived from the molecular formulas in Table 44.

AR_{s,a}:Atomic ratio of atom a in species s

Using the appropriate atomic weight of atom a (AWa), the molecular weight of all species s (MWₛ) is defined using Eq. (225).

AWₐ:Atomic weight of atom a

$${MW}_{s} = {\sum }_{a} {AW}_{a} \cdot {AR}_{s , a} \qquad ( 225 )$$

The proximate analysis for the biomass and coal species s is described by the total mass of moisture per unit mass of dry input (PAₛ^{M}) and the dry weight fractions (PA_{p,s}^{D}) of the ash, fixed carbon, and volatile matter components p.

PAₛ^{M}:Mass of water per unit mass of dry species s

PA_{p,s}^{D}:Dry mass fraction of proximate analysis component p in species s

In this study, switchgrass was chosen for the biomass feedstock and low-volatile bituminous coal was chosen for the coal feedstock.

Variables

Continuous variables are used in the mathematical model to describe the species molar flow rates (N_{u,u′,s}^{S}), the total molar flow rates (N_{u,u′}^{T}), the extent of reaction in a process unit (ξᵣ^{u}), the molar composition of a stream (x_{u,u′,s}^{S}), the split fraction of a stream between two units (sp_{u,u′}), the total stream enthalpy flow rate (H_{u,u′}^{T}), the heat lost from a unit (Qᵤ^{L}), the heat transferred to or absorbed from a unit (Qᵤ), the delivered cost of feedstock (Costₛ^{F}), the cost of CO₂ sequestration (Cost^{Seq}), the cost of electricity (Cost^{El}), and the levelized unit investment cost (Costᵤ^{U}). Note that the subscripts u and u′ are both used to denote an element of the set U and can be used interchangeably in the stream flow indices.

N_{u,u′,s}^{S}: Molar flow of species s from unit u to unit u′
N_{u,u′}^{T}: Total molarflowfrom unit u to unit u′
(ξᵣ^{u}): Extent of reaction r in unit u
   x_{u,u′,s}^{S}: Molar composition of species s from unit u to unit u′
   sp_{u,u′}: Split fraction of stream going from unit u to unit u′
   H_{u,u′}^{T}: Total enthalpy flow from unit u to unit u′
   Qᵤ^{L}: Heat lost from unit u
   Qᵤ: Heat transferred to or absorbed from unit u
   Costₛ^{F}: Total delivered cost of feedstock s
   Cost^{Seq}: Total sequestration cost of CO₂
   Cost^{El}: Total cost of electricity
   Costᵤ^{U}: Total levelized cost of unit u

Binary variables (yᵤ) are introduced to represent the logical use of a process unit u. These binary variables are only needed for specific process units since many of the units in the CBGTL process will always be required. The units that require binary variables include the biomass and coal gasifiers, the reverse water gas shift unit, the Fischer-Tropsch units, the autothermal reactor, and the gas turbine.

yᵤ: Logical existence of process unit u (i.e., it takes the value of one if unit u is selected and zero otherwise)

TABLE 43
Process units present in the CBGTL synthesis problem.

| Unit name | Unit index | Unit name | Unit index |
|---|---|---|---|
| Process inlets | | | |
| Inlet coal | IN_{COAL} | Inlet Natural gas | IN_{NC} |
| Inlet biomass | IN_{BIO} | Inlet air | IN_{AIR} |
| Inlet water | IN_{H2}O | Inlet butane | IN_{BUT} |
| Process outlets | | | |
| Outlet gasoline | OUT_{GAS} | Outlet diesel | OUT_{DIE} |
| Outlet kerosene | OUT_{KER} | Outlet ash | OUT_{ASH} |
| Outlet sulfur | OUT_{S} | Outlet scrubbed HCl | OUT_{SCR} |
| Outlet vent | OUT_{V} | Outlet propane | OUT_{PRO} |
| Outlet sequestered CO₂ | OUT_{CO}₂ | Outlet Wastewater | OUT_{WW} |
| Syngas generation | | | |
| Biomass dryer | BDR | Biomass dryer air heater | X_{BDR} |
| Biomass lockhopper | BLK | Biomass Gasifier | BGSₐ |
| First biomass vapor cyclone | BC₁ | Second biomass vapor cyclone | BD₂ |
| Tar cracker | TCK | Tar cracker splitter | SP_{TCK} |
| Tar cracker cooler | X_{TCK} | Coal dryer | CDR |
| Coal dryer air heater | X_{CDR} | Coal lockhopper | CLK |
| Coal gasifier | CGSₙ | First coal vapor cyclone | CC₁ |
| Second coal vapor cyclone | CC₂ | Second coal cyclone splitter | SP_{CC}₂ |
| Second coal cyclone cooler | X_{TCK} | | |
| Syngas cleaning | | | |
| Reverse water gas shift unit | RGSₙ | RGS effluent cooler | X_{RGS} |
| COS-HCN hydrolyzer | CHH | HCl scrubber | HSC |
| Acid gas flash vapor cooler | X_{AGF} | Acid gas flash 2-phase cooler | X_{AGF}ₙ |
| Acid gas flash unit | AGF | Acid gas thermal analyzer | X_{AGR} |
| Acid gas removal unit | AGR | First CO₂ compressor | CO₂C |
| CO₂ recycle compressor | CO₂RC | CO₂ sequestration compressor | CO₂SC |
| Acid gas compressor | AGC | | |
| Claus sulfur recovery | | | |
| Acid gas splitter | SP_{AG} | Acid gas preheater | X_{AG} |
| Claus combustor | CC | First sulfur converter | SC₁ |
| First sulfur separator | SS₁ | Second sulfur converter heater | X_{SC}₂ |
| Second sulfur converter | SC₂ | Second sulfur separator | SS₂ |
| Third sulfur converter hearer | X_{SC}₃ | Third sulfur converter | SC₃ |
| Third sulfur separator | SS₃ | Sulfur pit | SPT |
| Tail gas hydrolyzer | TGH | Tail gas flash vapor cooler | X_{TGF} |
| Tail gas flash 2-phase cooler | X_{TGF}ₙ | Tail gas flash unit | TGF |
| Tail gas compressor | TGC | | |
| Hydrocarbon production | | | |
| MTFTWGS-N | Iron MT fWGS nominal wax FT | MTFTWGS-M | Iron MT fWGS minimal wax TF |
| FT-ZSM5 | ZSM-5 hydrocarbon conversion unit | ZSM5F | ZSM-5 product fractionation |
| MEOHS | Methanol synthesis unit | MEOH-F | Methanol flash unit |
| MEDEG | Methanol degasser | MTG | Methanol to gasoline ZSM-5 reactor |
| MTO | Methanol to olefins ZSM-5 reactor | MTO-F | MTO fractionation |
| OGD | Olefins to gasoline/distillate | MTODF | OGD fractionation |
| Fischer-Tropsch compressor | FTC | Fischer-Tropsch splitter | SP_{FT} |
| Low-temperature preheater | X_{LTFT} | Low-temperature splitter | SP_{LTFT} |
| Low-temperature iron-based FT | LTFT | Low-temperature cobalt-based FT | LTFTRGS |
| High-temperature preheater | X_{HTFT} | High-temperature splitter | SP_{HTFT} |
| High-temperature iron-based FT | HTFT | High-temperature cobalt-based FT | HTFTRGS |
| Low-temperature effluent cooler | X_{LTFTC} | High-temperature effluent cooler | X_{HTFTC} |
| Water-soluble oxygenates separator | WSOS | Vapor-phase oxygenates separator | VPOS |
| Primary vapor-liquid-water separator | VLWS | | |
| Hydrocarbon recovery | | | |
| Hydrocarbon recovery column | HRC | Wax Hydrocracker | WHC |
| Distillate hydrotreater | DHT | Kerosene hydrotreater | KHT |
| Naphtha hydrotreater | NHT | Naphtha reformer | NRF |
| C₄ Isomerizer | C₄I | C₅-C₆ Isomerizer | C₅₆I |
| C₃-C₄-C₅ Alkylation unit | C₃₄₅A | Saturated gas plant | SGP |
| Diesel blender | DBL | Gasoline blender | GBL |
| HCKO1 | Mixed hydrocarbon knockout 1 | HCKO2 | Mixed hydrocarbon knockout 2 |
| DEETH | De-ethanizer | ABS-COL | Absorber column |
| CO₂SEP | I-stage Rectisol CO₂ separation | STA-COL | Stabilizer column |
| ALK-UN | HF alkylation unit | LPG-ALK | LPG/Alkylate splitter |
| SP-COL | Splitter column | | |
| Recycle gas treatment | | | |
| Light gas compressor | LGC | Light gas splitter | SP_{LG} |
| Auto-thermal reactor | ATRₙ | Auto-thermal reactor splitter | SP_{ATR}ₙ |
| Fuel combustor | FCM | Fuel combuster effluent cooler | X_{FCM} |
| Fluel combustor flash unit | FCF | First gas turbine air compressor | GTAC₁ |

The subscript n corresponds to multiple forms of the same process unit, each with a distinct set of operating conditions or ratios of feedstock. Distinct process units are used in lieu of continuous variables representing the process operating conditions. This will prevent the use of bilinear terms when specifying feedstock ratios or highly non-linear equations when specifying equilibrium constants or species enthalpies.

TABLE 44
Species present in the CBGTL synthesis problem.

| Species name | Species index | Species name | Species index | Species name | Species index |
|---|---|---|---|---|---|
| Acid gases | | | | | |
| Sulfur dioxide | SO₂ | Hydrogen sulfur | H₂S | Carbonyl sulfide | COS |
| Hydrogen cyanide | HCN | Ammonia | NH₃ | Hydrogen chloride | HCl |
| Carbon dioxide | CO₂ | | | | |
| Light non-hydrocarbon gases | | | | | |
| Oxygen | O₂ | Nitrogen | N₂ | Argon | Ar |
| Nitric oxide | NO | Nitrous oxide | N₂O | Water | H₂O |
| Carbon monoxide | CO | Hydrogen | H₂ | | |
| Hydrocarbons | | | | | |
| Methane | CH₄ | Acetylene | C₂H₂ | Ethylene | C₂H₄ |
| Ethane | C₂H₆ | Propylene | C₃H₆ | Propane | C₃H₈ |
| Isobutylene | iC₄H₈ | 1-Butene | nC₄H₈ | Isobutane | iC₄H₁₀ |
| n-Butane | nC₄H₁₀ | 1-Pentene | C₅H₁₀ | 2-Methylbutane | iC₅H₁₂ |
| n-Pentane | nC₅H₁₂ | 1-Hexene | C₆H₁₂ | 2-Methylpentane | iC₆H₁₄ |
| n-Hexane | nC₆H₁₄ | 1-Heptene | C₇H₁₄ | n-Heptane | C₇H₁₆ |
| 1-Octene | C₈H₁₆ | n-Octane | C₈H₁₈ | 1-Nonene | C₉H₁₈ |
| n-Nonane | C₉H₂₀ | 1-Decene | C₁₀H₂₀ | n-Decane | C₁₀H₂₂ |
| 1-Undecene | C₁₁H₂₂ | n-Undecane | C₁₁H₂₄ | 1-Dodecene | C₁₂H₂₄ |
| n-Dodecane | C₁₂H₂₀ | 1-Tridecene | C₁₃H₂₆ | n-Tridecane | C₁₃H₂₈ |
| 1-Tetradecene | C₁₄H₂₈ | n-Tetradecane | C₁₄H₃₀ | 1-Pentadecene | C₁₅H₃₀ |
| n-Pentadecane | C₁₅H₃₂ | 1-Hexadecene | C₁₆H₃₂ | n-Hexadecane | C₁₆H₃₄ |
| 1-Heptadecene | C₁₇H₃₄ | n-Heptadecane | C₁₇H₃₆ | 1-Octadecene | C₁₈H₃₆ |
| n-Octadecane | C₁₈H₃₈ | 1-Nonadecene | C₁₉H₃₈ | n-Nonadecane | C₁₉H₄₀ |
| 1-Eicosene | C₂₀H₄₀ | n-Eicosane | C₂₀H₄₂ | C₂₁ Pseudocomponent | C₂₁OP |
| C₂₂ Pseudocomponent | C₂₂OP | C₂₃ Pseudocomponent | C₂₃OP | C₂₄ Pseudocomponent | C₂₄OP |
| C₂₅ Pseudocomponent | C₂₅OP | C₂₆ Pseudocomponent | C₂₆OP | C₂₇ Pseudocomponent | C₂₇OP |
| C₂₈ Pseudocomponent | C₂₈OP | C₂₉ Pseudocomponent | C₂₉OP | C₃₀₊ Pseudocomponent | C₃₀Wax |
| VP Oxygenate | OXVAP | HP Oxygenate | OXHC | AP Oxygenate | OXH2O |
| Products | | | | | |
| Gasoline | GAS | Diesel | DIE | Kerosene | KER |
| Solid sulfur | S | | | | |
| Non-conventional components | | | | | |
| Biomass | e.g. Perennial | Coal | e.g. LV-bituminous | Gasifier char | Char |
| Feedstock ash | Ash | | | | |

The molecular formula of the pseudocomponent hydrocarbons and oxygenates are given by Bechtel. The formula for the biomass and coal species are derived from the ultimate analysis assuming that the “atomic” weight of ash is 1.0 g/mol.

General constraints

Mass balances

Species balances

$${\sum }_{( {u}^{′} , u ) ∈ UC} {N}_{{u}^{′} , u , s}^{S} - {\sum }_{( u , r , {s}^{′} ) ∈ {R}^{U}} \frac{{v}_{r , s}}{{v}_{r , {s}^{′}}} \cdot {ξ}_{r}^{u} - {\sum }_{( u , {u}^{′} ) ∈ UC} {N}_{u , {u}^{′} , s}^{S} = 0 \\ ∀ s ∈ {S}_{u}^{U} , u ∈ {U}_{Sp}^{Bal} \qquad ( 226 )$$

Extent of reaction

$${ξ}_{r}^{u} - {fc}_{r}^{u} \cdot {\sum }_{( {u}^{′} , u , s ) ∈ {S}^{UF}} {N}_{{u}^{′} , u , s}^{S} = 0 \\ ∀ ( u , r , s ) ∈ {R}^{U} \qquad ( 227 )$$

Atom balances

$${\sum }_{( {u}^{′} , u , s ) ∈ {S}^{UF}} {AR}_{s , a} \cdot {N}_{{u}^{′} , u , s}^{S} - {\sum }_{( u , {u}^{′} , s ) ∈ {S}^{UF}} {AR}_{s , a} \cdot {N}_{u , {u}^{′} , s}^{S} = 0 \\ ∀ a ∈ {A}_{u}^{U} , u ∈ {U}_{At}^{Bal} \qquad ( 228 )$$

Total mole balance

$${N}_{{u}^{′} , u}^{T} - {\sum }_{( u , {u}^{′} , s ) ∈ {S}^{UF}} {N}_{{u}^{′} , u , s}^{S} = 0 \\ ∀ ( u , {u}^{′} ) ∈ UC \qquad ( 229 )$$

Process splitters

Set unit split fractions

N_{u,u′,s}^{S}−xᵤ_{_{I}}_{,u,s}^{S}·N_{u,u′}^{T}=0∀(u,u′,s)∈S^{UF},u∈U_{Sp} (230)

Split fractions sum to 1

$${\sum }_{( u , {u}^{′} , s ) ∈ {S}^{UF}} {x}_{u , {u}^{′} , s}^{S} - 1 = 0 \\ ∀ ( u , {u}^{′} ) ∈ {UC}_{Comp} \qquad ( 231 )$$

Flash units

Upper bound of liquid phase split fraction

$${x}_{u , {u}_{L} , s}^{S} - min \{ 1 , \frac{1}{{K}_{u , s}^{VLE}} \} \leq 0 \\ ∀ ( u , {u}_{L} , s ) ∈ {S}^{UF} , u ∈ {U}_{Fl} \qquad ( 232 )$$

Upper bound of vapor phase split fraction

x_{u,u}_{_{V}}_{,s}^{S}−min(1,K_{u,s}^{VLE})≦0∀(u,u_{V},s)∈S^{UF},u∈U_{Fl} (233)

Set liquid phase split fraction

x_{u,u}_{_{L}}_{,s}^{S}·N_{u,u}_{_{L}}^{T}−N_{u,u}_{_{L}}_{,s}^{S}=0∀u∈U_{Fl} (234)

Set vapor phase split fraction

x_{u,u}_{_{V}}_{,s}^{S}·N_{u,u}_{_{V}}^{T}−N_{u,u}_{_{V}}_{,s}^{S}=0∀u∈U_{Fl} (235)

Set phase equilibrium

x_{u,u}_{_{V}}_{,s}^{S}−K_{u,s}^{VLE}·x_{u,u}_{_{L}}_{,s}^{S}=0∀u∈U_{Fl} (236)

Heat balances

Conservation of energy

$${\sum }_{( u , {u}^{′} ) ∈ UC} {H}_{u , {u}^{′}}^{T} - {\sum }_{( {u}^{′} , u ) ∈ UC} {H}_{{u}^{′} , u}^{T} - {Q}_{u} - {Q}_{u}^{L} - Wu = 0 \\ ∀ u ∈ \frac{U}{{U}_{Agg}} \qquad ( 237 )$$

Total heat balance

$${H}_{u , {u}^{′}}^{T} - {\sum }_{( u , {u}^{′} , s ) ∈ {S}^{UF}} {H}_{u , {u}^{′} , s}^{S} = 0 \\ ∀ ( u , {u}^{′} ) ∈ UC \qquad ( 238 )$$

Logical unit existence

Bound on molar flows

$${\sum }_{( {u}^{′} , u ) ∈ UC} {N}_{{u}^{′} , u}^{T} - {UB}_{u}^{N} \cdot {y}_{u} \leq 0 \\ ∀ u ∈ {U}^{Ex} \qquad ( 239 )$$

Upper bound on inlet enthalpy flow

H_{u′,u}^{T}−UB_{u′,u}^{H}·yᵤ≦0∀(u′,u)∈UC,u∈U^{Ex} (240)

Lower bound on inlet enthalpy flow

LB_{u′,u}^{H}·yᵤ−H_{u′,u}^{T}≦0∀(u′,u)∈UC,u∈U^{Ex} (241)

Upper bound on outlet enthalpy flow

H_{u,u′}^{T}−UB_{u′,u}^{H}·yᵤ≦0∀(u,u′)∈UC,u∈U^{Ex} (242)

Lower bound on outlet enthalpy flow

LB_{u,u′}^{H}·yᵤ−H_{u,u′}^{T}≦0∀(u,u′)∈UC,u∈U^{Ex} (243)

Process inlets
Feedstock moisture content

Set biomass moisture content from proximate analysis

$${M}_{u , {u}^{′} , {H}_{2} O}^{S} - {\sum }_{s ∈ {S}_{Bio}} {PA}_{s}^{M} \cdot {M}_{u , {u}^{′} , s}^{S} = 0 \\ ( u , {u}^{′} ) = ( {IN}_{BIO} , BDR ) \qquad ( 244 )$$

Set coal moisture content from proximate analysis

$${M}_{u , {u}^{′} , {H}_{2} O}^{S} - {\sum }_{s ∈ {S}_{Coal}} {PA}_{s}^{M} \cdot {M}_{u , {u}^{′} , s}^{S} = 0 \\ ( u , {u}^{′} ) = ( {IN}_{COAL} , CDR ) \qquad ( 245 )$$

Known stream compositions

Set stream compositions for inlet streams

N_{u,u′,s}^{S}−x_{u,s}^{K}·N_{u,u′}^{T}=0∀(u,u′,s)∈S^{UF},u={IN_{AIR},IN_{NG},IN_{BUT}} (246)

Coal to natural gas ratio

Set coal to natural gas inlet ratio based on lower heating value ratios

$${\sum }_{s ∈ {S}_{Coal}} {N}_{{IN}_{COAL} , CDR , s}^{S} \cdot {LHV}_{s} - {LHV}_{CG}^{Rat} \cdot {LHV}_{NG} \cdot {\sum }_{( {IN}_{NG} , u ) ∈ UC} {N}_{{IN}_{NG} , u}^{T} = 0 \qquad ( 247 )$$

Greenhouse gas emissions reduction

Set reduction from petroleum based process

GHG_{CBGTL}−GHG_{Red}·GHG_{Per}=0 (248)

Sum emissions from CBGTL components

GHG_{CBGTL}−GHG^{Seq}−GHG^{Proc}−GHG^{Feed}=0 (249)

Set emissions from feedstock acquisition

$${GHG}^{Feed} - {\sum }_{u ∈ {U}_{In}} {\sum }_{( u , {u}^{′} , s ) ∈ {S}^{UF}} {GHG}_{s}^{T} \cdot {M}_{u , {u}^{′} , s}^{S} = 0 \qquad ( 250 )$$

Set emissions from CO₂ sequestration

$${GHG}^{Seq} - {GHG}_{{CO}_{2}}^{T} \cdot {MW}_{{CO}_{2}} \cdot {N}_{{CO}_{2} SC , {OUT}_{{CO}_{2}} , {CO}_{2}} = 0 \qquad ( 251 )$$

Set emissions from CO₂ venting

$${GHG}^{Proc} - {MW}_{{CO}_{2}} \cdot {N}_{{CO}_{2} R , {OUT}_{V} , {CO}_{2}} = 0 \qquad ( 252 )$$

Process outlet fuel ratios

Set gasoline to diesel output ratio

MW_{GAS}·N_{GBL,OUT}_{_{GAS}}_{,GAS}^{S}−Rat_{G-D}·MW_{DIE}·N_{DBL,OUT}_{_{DIE}}_{,DIE}^{S}=0 (253)

Set diesel to kerosene output ratio

MW_{DIE}·N_{DBL,OUT}_{_{DIE}}_{,DIE}^{S}−Rat_{D-K}·MW_{KER}·N_{KHT,OUT}_{_{KER}}_{,KER}^{S}=0 (254)

Syngas generation
Biomass/coal driers

Upper bound for biomass drier activation

M_{u,u′,H}_{₂}_{O}^{S}−MT_{Bio}·M_{u,u′}^{T}−UB·yᵤ≦0(u,u′)=(IN_{BIO},BDR) (255)

Upper bound for coal drier activation

M_{u,u′,H}_{₂}_{O}^{S}−MT_{Coal}·M_{u,u′}^{T}−UB·yᵤ≦0(u,u′)=(IN_{COAL},CDR) (256)

Lower bound for biomass drier activation

MT_{BIO}·M_{u,u′}^{T}−M_{u,u′,H}_{₂}_{O}^{S}−UB·(1−yᵤ)≦0(u,u′)=(IN_{BIO},BDR) (257)

Lower bound for coal drier activation

MT_{BIO}·M_{u,u′}^{T}−M_{u,u′,H}_{₂}_{O}^{S}−UB·(1−yᵤ)≦0(u,u′)=(IN_{COAL},CDR) (258)

Upper bound for biomass drier moisture evaporation

MT_{BIO}·M_{u,u′}^{T}−M_{u,u′,H}_{₂}_{O}^{S}−UB·(1−yᵤ)≦0(u,u′)=(BDR,BLK) (259)

Lower bound for biomass drier moisture evaporation

M_{u,u′,H}_{₂}_{O}^{S}−MT_{Bio}·M_{u,u′}^{T}−UB·(1−yᵤ)≦0(u,u′)=(BDR,BLK) (260)

Upper bound for coal drier moisture evaporation

MT_{Coal}·M_{u,u′}^{T}−M_{u,u′,H}_{₂}_{O}^{S}−UB·(1−yᵤ)≦0(u,u′)=(CDR,CLK) (261)

Lower bound for coal drier moisture evaporation

M_{u,u′,H}_{₂}_{O}^{S}−MT_{Coal}·M_{u,u′}^{T}−UB·(1−yᵤ)≦0(u,u′)=(CDR,CLK) (262)

Gasifier lockhoppers

Set CO₂ lockhopper flow rate

$${M}_{{CO}_{2} {C}_{2} , BLK , {CO}_{2}}^{S} - {mf}_{u} \cdot {\sum }_{s ∈ {S}_{Bio}} {M}_{BDR , BLK , s}^{S} = 0 \qquad ( 263 )$$

Biomass gasifier

Water-gas-shift equilibrium

N_{u,BC1,CO}·N_{u,BC1,H}_{₂}_{O}−Kᵤ^{RGS}·N_{u,BC1,CO}_{₂}·N_{u,BC1,H}_{₂}=0∀u∈U_{BGS} (264)

Hydrocarbon conversion fraction

$${M}_{u , BC 1 , s} - {\sum }_{( {u}^{′} , u , s ) ∈ {S}^{UF}} {cf}_{u , s}^{HC} \cdot {M}_{s}^{S , Calc} = 0 \\ ∀ s ∈ {S}_{HC} , u ∈ {U}_{BGS} \qquad ( 265 )$$

Hydrocarbon generation from pyrolysis

$${M}_{s}^{S , Calc} - {\sum }_{{s}^{′} ∈ {S}_{Bio}} {\sum }_{( {u}^{′} , u , {s}^{′} ) ∈ {S}^{UF}} {Pyr}_{s , {s}^{′}}^{HC} \cdot {M}_{{u}^{′} , u , {s}^{′}}^{S} - {\sum }_{( {u}^{′} , u ) ∈ UC} {M}_{{u}^{′} , u , s}^{S} = 0 \\ u ∈ {U}_{BGS} \qquad ( 266 )$$

Set ratio of NO to N₂O

$${N}_{u , BC 1 , NO} - {sr}_{u , \frac{NO}{{N}_{2} O}} \cdot {N}_{u , BC 1 , {N}_{2} O} = 0 \\ ∀ u ∈ {U}_{BGS} \qquad ( 267 )$$

Set ratio of HCN to NH₃

$${N}_{u , BC 1 , HCN} - {sr}_{u , \frac{HCN}{{NH}_{3}}} \cdot {N}_{u , BC 1 , {NH}_{3}} = 0 \\ ∀ u ∈ {U}_{BGS} \qquad ( 268 )$$

Set amount input nitrogen to NH₃ and N₂

$${N}_{u , BC 1 , {NH}_{3}} + 2 \cdot {N}_{u , BC 1 , {N}_{2}} - {nf}_{u} \cdot {\sum }_{( u , BC 1 , s ) ∈ {S}^{UF}} {N}_{u , BC 1 , s}^{S} \cdot {AR}_{s , N} = 0 \\ ∀ u ∈ {U}_{BGS} \qquad ( 269 )$$

Set ratio of NH₃ to N₂

N_{u,BC1,NH}_{₃}−·(a_{u,N}_{₂}¹+a_{u,N}_{₂}²·Tᵤ)·(N_{u,BC1,NH}_{₃}+2·N_{u,BC1,N}_{₂})=0∀u∈U_{BGS} (270)

Set ratio of COS to H₂S

$${N}_{u , BC 1 , COS} - {sr}_{u , \frac{COS}{{H}_{2} S}} \cdot {N}_{u , BC 1 , {H}_{2} S} = 0 \\ ∀ u ∈ {U}_{BGS} \qquad ( 271 )$$

Amount of char production

$${MW}_{Char} \cdot {N}_{u , BC 1 , Char}^{s} - ( {a}_{u , Char}^{1} + {a}_{u , Char}^{2} \cdot {T}_{u} ) \cdot {\sum }_{s ∈ {S}_{Bio}} {MW}_{s} \cdot {N}_{BLK , u , s}^{s} = 0 \\ ∀ u ∈ {U}_{BGS} \qquad ( 272 )$$

Rate of ash removal

$${N}_{u , {OUT}_{ASH} , ASH}^{S} - {sf}_{u , ASH} \cdot {\sum }_{( {u}^{′} , u ) ∈ UC} {N}_{{u}^{′} , u , Ash}^{S} = 0 \\ ∀ u ∈ {U}_{BGS} \qquad ( 273 )$$

Gasifier heat loss

$${Q}_{u}^{L} + {hl}_{u} \cdot {\sum }_{s ∈ {S}_{Bio}} {M}_{BLK , u , s}^{S} \cdot L H {V}_{s} = 0 \\ ∀ u ∈ {U}_{BGS} \qquad ( 274 )$$

Logical use of one gasifier temperature

$${\sum }_{u ∈ {U}_{BGS}} {y}_{u} - 1 = 0 \qquad ( 275 )$$

Biomass gasifier solids

Removal of solids from first cyclone

rf_{BC1}·N_{BGS,BC1}^{T}−N_{BC1,BGS}^{T}=0 (276)

Removal of solids from second cyclone

rf_{BC2}·N_{BC1,BC2}^{T}−N_{BC2,BGS}^{T}=0 (277)

Coal gasifier

Set CO₂ lockhopper flow rate

$${M}_{{SP}_{CO 2} , CLK , {CO}_{2}}^{S} - {mf}_{u} \cdot {\sum }_{s ∈ {S}_{Coal}} {M}_{CDR , CLK , s}^{S} = 0 \qquad ( 278 )$$

Water-gas-shift equilibrium

N_{u,CC}_{₁}_{,CO}·N_{u,CC}_{₁}_{,H}_{₂}_{O}−Kᵤ^{RGS}·N_{u,CC}_{₁}_{,CO}_{₂}·N_{u,CC}_{₁}_{,H}_{₂}=0∀u∈U_{CGS} (279)

Hydrocarbon conversion fraction

$${M}_{u , {CC}_{1} , 5} - {\sum }_{( {u}^{′} , u , s ) ∈ {S}^{UF}} {cf}_{u , s}^{HC} \cdot {M}_{s}^{S , Calc} = 0 \\ ∀ s ∈ {S}_{HC} , u ∈ {U}_{CGS} \qquad ( 280 )$$

Hydrocarbon generation from pyrolysis

$${M}_{s}^{S , Calc} - {\sum }_{{s}^{′} {∈}_{Coal}} {\sum }_{( {u}^{′} , u , {s}^{′} ) ∈ {S}^{UF}} {Pyr}_{s , {s}^{′}}^{HC} \cdot {M}_{{u}^{′} , u , {s}^{′}}^{S} - {\sum }_{( {u}^{′} , u ) ∈ UC} {M}_{{u}^{′} , u , s}^{S} = 0 \\ u ∈ {U}_{CGS} \qquad ( 281 )$$

Set ratio of NO to N₂O

$${N}_{u , {CC}_{1} , NO} - {sr}_{\frac{NO}{{N}_{2} O}} \cdot {N}_{u , {CC}_{1} , {N}_{2} O} = 0 \\ ∀ u ∈ {U}_{CGS} \qquad ( 282 )$$

Set ratio of HCN to NH₃

$${N}_{u , {CC}_{1} , HCN} - {sr}_{\frac{HCN}{{NH}_{3}}} \cdot {N}_{u , {CC}_{1} , {NH}_{3}} = 0 \\ ∀ u ∈ {U}_{CGS} \qquad ( 283 )$$

Set amount input nitrogen to NH₃ and N₂

$${N}_{u , {CC}_{1} , {NH}_{3}} + 2 \cdot {N}_{u , {CC}_{1} , {N}_{2}} - {nf}_{u} \cdot {\sum }_{( u , {CC}_{1} , s ) ∈ {S}^{UF}} {N}_{u , {CC}_{1} , s}^{S} \cdot {AR}_{s , N} = 0 \\ ∀ u ∈ {U}_{CGS} \qquad ( 284 )$$

Set ratio of NH₃ to N₂

N_{u,CC}_{₁}_{,NH}_{₃}−(a_{u,N}_{₂}¹+a_{u,N}_{₂}²·Tᵤ)·(N_{u,CC}_{₁}_{,NH}_{₃}+2·N_{u,CC}_{₁}_{,N}_{₂})=0∀u∈U_{CGS} (285)

Set ratio of COS to H₂S

$${N}_{u , {CC}_{1} , {H}_{2} S} - {sr}_{u , \frac{COS}{{H}_{2} S}} \cdot {N}_{u , {CC}_{1} , COS} = 0 \\ ∀ u ∈ {U}_{CGS} \qquad ( 286 )$$

Amount of char production

$${MW}_{Char} \cdot {N}_{u , {CC}_{1} , Char}^{S} - ( {a}_{u , Char}^{1} + {a}_{u , Char}^{2} \cdot {T}_{u} ) \cdot {\sum }_{s ∈ {S}_{Coal}} {MW}_{s} \cdot {N}_{CLK , u , s}^{s} = 0 \\ ∀ u ∈ {U}_{CGS} \qquad ( 287 )$$

Rate of ash removal

$${N}_{u , {OUT}_{ASH} , Ash}^{S} - {sf}_{u , Ash} \cdot {\sum }_{( {u}^{′} , u ) ∈ UC} {N}_{{u}^{′} , u , Ash}^{s} = 0 \\ ∀ u ∈ {U}_{CGS} \qquad ( 288 )$$

Gasifier heat loss

$${Q}_{u}^{L} + {hl}_{u} \cdot {\sum }_{s ∈ {S}_{Coal}}^{ } {M}_{CLK , u , s}^{S} \cdot {LHV}_{s} = 0 \\ ∀ u ∈ {U}_{CGS} \qquad ( 289 )$$

Logical use of one gasifier temperature

$${\sum }_{u ∈ {U}_{CGS}}^{ } {y}_{u} - 1 = 0 \qquad ( 290 )$$

Coal gasifier solids

Removal of solids from first cyclone

rf_{CC1}·N_{CGS,CC1}^{T}−N_{CC1,CGS}^{T}=0 (291)

Removal of solids from second cyclone

rf_{CC2}·N_{CC1,CC2}^{T}−N_{CC2,CGS}^{T}=0 (292)

Syngas cleaning
Reverse water-gas-shift unit

Bypass of inert species

$${\sum }_{( {u}^{′} , u , s ) ∈ {S}^{UF}}^{ } {N}_{{u}^{′} , u , s}^{S} - {\sum }_{( u , {u}^{′} , s ) ∈ {S}^{UF}}^{ } {N}_{u , {u}^{′} , s}^{S} = 0 \\ ∀ s ∈ {S}_{u}^{In} , u ∈ {U}_{RGS} \qquad ( 293 )$$

Water-gas-shift equilibrium

N_{u′,u,CO}·N_{u′,u,H}_{₂}_{O}−K_{u′}^{RGS}·N_{u′,u,CO}_{₂}·N_{u′,u,H}_{₂}=0∀u′∈U_{RGS},u=X_{RGS} (294)

Logical use of unit with at most one temperature

$${\sum }_{u ∈ {U}_{RGS}}^{ } {y}_{u} - 1 \leq 0 \qquad ( 295 )$$

COS—HCN hydrolyzer

Bypass of inert species

$${\sum }_{( {u}^{′} , u , s ) ∈ {S}^{UF}}^{ } {N}_{{u}^{′} , u , s}^{S} - {\sum }_{( u , {u}^{′} , s ) ∈ {S}^{UF}}^{ } {N}_{u , {u}^{′} , s}^{S} = 0 \\ ∀ s ∈ {S}_{u}^{In} , u ∈ {U}_{CHH} \qquad ( 296 )$$

COS—H₂S equilibrium

N_{u′,u,COS}·N_{u′,u,H}_{₂}_{O}−K_{u′}^{COS}·N_{u′,u,CO}_{₂}·N_{u′,u,H}_{₂}_{S}=0(u′,u)=(CHH,HSC) (297)

HCN—NH₃ equilibrium

N_{u′,u,HCN}·N_{u′,u,H}_{₂}_{O}−K_{u′}^{HCN}·N_{u′,u,CO}·N_{u′,u,NH}_{₃}=0(u′,u)=(CHH,HSC) (298)

Acid gas recovery
Set CO₂ molar fraction in clean output

N_{AGR,SP}_{_{AGR}}_{,CO}_{₂}^{S}−rf_{AGR}·N_{AGR,SP}_{_{CG}}^{T}=0 (299)

Set CO₂ output flow rates

N_{AGR,CO}_{₂}_{C}^{T}−sf_{AGR}·(N_{AGR,CO}_{₂}_{C}^{T}+N_{AGR,MX}_{_{CO2RC}}^{T})=0 (300)

Claus sulfur recovery

Set inlet combustor oxygen level

$${\sum }_{( u , CC ) ∈ UC}^{ } {N}_{u , CC , {O}_{2}}^{S} - {er}_{CC} \cdot {\sum }_{( u , CC , s ) ∈ {S}^{UF}}^{ } {N}_{c , CC , s}^{S} \cdot {sor}_{s} = 0 \qquad ( 301 )$$

Hydrocarbon production

### Fischer-Tropsch

Set ratio of H₂ to CO in cobalt-based inlet

$${\sum }_{( {u}^{′} , u , {H}_{2} ) ∈ {S}^{UF}}^{ } {FTR}_{u , {H}_{2}} - 2 \cdot {\sum }_{( {u}^{′} , u , CO ) ∈ {S}^{UF}}^{ } {FTR}_{u , CO} = 0 \\ ∀ u ∈ {U}_{CoFT} \qquad ( 302 )$$

Set ratio of H₂ to CO and CO₂ in iron-based inlet

$${\sum }_{( {u}^{′} , u , {H}_{2} ) ∈ {S}^{UF}}^{ } {FTR}_{u , {H}_{2}} - 2 \cdot {\sum }_{( {u}^{′} , u , CO ) ∈ {S}^{UF}}^{ } {FTR}_{u , CO} - 3 \cdot {\sum }_{( {u}^{′} , u , {CO}_{2} ) ∈ {S}^{UF}}^{ } {FTR}_{u , {CO}_{2}} = 0 \\ ∀ u ∈ {U}_{IrFT} \qquad ( 303 )$$

Adjust weight fraction of C₁ species

$${W}_{1} = \frac{1}{2} ( 1 - {\sum }_{n = 5}^{∞} {W}_{n} ) \qquad ( 304 )$$

Adjust weight fraction of C₂ species

$${W}_{2} = \frac{1}{6} ( 1 - {\sum }_{n = 5}^{∞} {W}_{n} ) \qquad ( 305 )$$

Adjust weight fraction of C₃ species

$${W}_{3} = \frac{1}{6} ( 1 - {\sum }_{n = 5}^{∞} {W}_{n} ) \qquad ( 306 )$$

Adjust weight fraction of C₄ species

$${W}_{4} = \frac{1}{6} ( 1 - {\sum }_{n = 5}^{∞} {W}_{n} ) \qquad ( 307 )$$

Set weight fraction of Cn species from Anderson-Schultz-Flory distribution

Wₙ=n(1−α)²αⁿ⁻¹∀5≦n≦29 (308)

Set weight fraction of wax

$${W}_{Wax} = {\sum }_{n = 30}^{∞} {n ( 1 - α )}^{2} {α}^{n - 1} \qquad ( 309 )$$

Set carbon distribution from weight fractions

$${cr}_{n} = \frac{n \cdot {W}_{n}}{{\sum }_{n = 1}^{29} n \cdot {W}_{n} + {n}_{Wax} \cdot {W}_{Wax}} \qquad ( 310 )$$

Set exactly one low-temperature unit

y_{LTFT}+y_{LTFTRGS}−1=0 (311)

Set exactly one high-temperature unit

y_{HTFT}+y_{HTFTRGS}−1=0 (312)

Aqueous phase oxygenates separator

Removal of aqueous phase oxygenates

N_{WSOS,VLWS,s}^{S}=0∀s∈S_{APO} (313)

Vapor phase oxygenates separator

Removal of vapor phase oxygenates

N_{VPOS,HRC,s}^{S}=0∀s∈S_{VPO} (314)

Hydrocarbon upgrading
Hydrocarbon upgrading units

Set carbon distribution fractions of total input

$${N}_{u , {u}^{′} , s}^{S} \cdot {AR}_{s , C} - {cf}_{u , {u}^{′} , s} \cdot {\sum }_{( {u}^{″} , u , {s}^{′} ) ∈ {S}^{UF}}^{ } {N}_{{u}^{″} , u , {s}^{′}}^{S} \cdot {AR}_{{s}^{′} , C} = 0 \\ ∀ u ∈ {U}_{UG} , ( u , {u}^{′} , s ) ∈ {s}^{UF} \qquad ( 315 )$$

Saturated gas plant

Set fractional recovery of light gases

$${N}_{SGP , {C}_{4} I , s}^{S} - {rf}_{s} \cdot {\sum }_{( u , SGP , s ) ∈ {S}^{UF}}^{ } {N}_{u , SGP , s}^{S} = 0 ∀ s ∈ {S}_{{C}_{4}} \qquad ( 316 )$$

Recycle gas treatment
Fuel combustor

Set inlet combustor oxygen level

$${\sum }_{( u , FCM ) ∈ UC}^{ } {N}_{c , FCM , {O}_{2}}^{S} - {er}_{FCM} \cdot {\sum }_{( {SP}_{LG} , FCM , s ) ∈ {S}^{UF}}^{ } {N}_{{SP}_{LG} , FCM , s}^{S} \cdot {sor}_{s} = 0 \qquad ( 317 )$$

Auto-thermal reactor

Logical use of one temperature

$${\sum }_{u ∈ {U}_{ATR}}^{ } {y}_{u} - 1 = 0 \qquad ( 318 )$$

Water-gas-shift equilibrium

N_{u,u′,CO}_{₂}^{S}·N_{u,u′,H}_{₂}^{S}−Kᵤ^{RGS}·N_{u,u′,CO}^{S}·N_{u,u′,H}_{₂}_{O}^{S}=0∀(u,u′)∈UC,u∈U_{ATR} (319)

CH₄ Steam reforming equilibrium

x_{u,u′,CO}^{S}·x_{u,u′,H}_{₂}^{S}^{³}−K_{u,CH}_{₄}^{SR}·x_{u,u′,CH}_{₄}^{S}·x_{u,u′,H}_{₂}_{O}^{S}=0∀(u,u′)∈UC,u∈U_{ATR} (320)

C₂H₂ steam reforming equilibrium

$${x}_{u , {u}^{′} , {C}_{2} {H}_{4}}^{S} - \frac{{K}_{u , {C}_{2} {H}_{2}}^{SR}}{{K}_{u , {C}_{2} {H}_{4}}^{SR}} \cdot {x}_{u , {u}^{′} , {C}_{2} {H}_{2}}^{S} \cdot {x}_{u , {u}^{′} , {H}_{2}}^{S} = 0 \\ ∀ ( u , {u}^{′} ) ∈ UC , u ∈ {U}_{ATR} \qquad ( 321 )$$

C₂H₄ steam reforming equilibrium

$${x}_{u , {u}^{′} , {C}_{2} {H}_{4}}^{S} - \frac{{K}_{u , {C}_{2} {H}_{2}}^{SR}}{{K}_{u , {C}_{2} {H}_{4}}^{SR}} \cdot {x}_{u , {u}^{′} , {C}_{2} {H}_{2}}^{S} \cdot {x}_{u , {u}^{′} , {H}_{2}}^{S} = 0 \\ ∀ ( u , {u}^{′} ) ∈ UC , u ∈ {U}_{ATR} \qquad ( 322 )$$

C₂H₆ Steam reforming equilibrium

$${x}_{u , {u}^{′} , {C}_{2} {H}_{6}}^{S} - \frac{{K}_{u , {C}_{2} {H}_{4}}^{SR}}{{K}_{u , {C}_{2} {H}_{6}}^{SR}} \cdot {x}_{u , {u}^{′} , {C}_{2} {H}_{4}}^{S} \cdot {x}_{u , {u}^{′} , {H}_{2}}^{S} = 0 \\ ∀ ( u , {u}^{′} ) ∈ UC , u ∈ {U}_{ATR} \qquad ( 323 )$$

Bypass of inert species

$${\sum }_{( {u}^{′} , u , s ) ∈ {S}^{UF}}^{ } {N}_{{u}^{′} , u , s}^{S} - {\sum }_{( u , {u}^{′} , s ) ∈ {S}^{UF}}^{ } {N}_{u , {u}^{′} , s}^{S} = 0 \\ ∀ u ∈ {U}_{ATR} , s ∈ {S}_{ATR}^{In} \qquad ( 324 )$$

Gas turbine

Set air leakage from first compressor

N_{GTAC}_{₁}_{,OUT}_{_{V}}_{,s}^{S}−lk_{GTAC}_{₁}·N_{IN}_{_{AIR}}_{,CTAC}_{₁}_{,s}^{S}=0∀(GTAC₁,s)∈S^{U} (325)

Set air bypass from first compressor

N_{GTAC}_{₁}_{,GT}_{₂}_{,s}^{S}−by_{GTAC}_{₁}·N_{IN}_{_{AIR}}_{,GTAC}_{₁}_{,s}^{S}=0∀(GTAC₁,s)∈S^{U} (326)

Set inlet oxygen flow rate in combustor

$${er}_{GTC} \cdot {\sum }_{( u , GTC , s ) ∈ {S}^{UF}}^{ } {sor}_{s} \cdot {N}_{u , GTC , s}^{S} - {\sum }_{( u , GTC , s ) ∈ {S}^{UF}}^{ } {N}_{u , GTC , {O}_{2}}^{S} = 0 \qquad ( 327 )$$

Set heat loss in combustor

Q_{GTC}^{L}−hl_{GTC}·(H_{SP}_{_{LG}}_{,GTC}^{T}−H_{X}_{_{GTF}}_{,GTF}^{T})=0 (328)

Wastewater treatment
Sour stripper

Set recovery fraction of H₂O in bottoms

$${N}_{SS , {SP}_{SS} , {H}_{2} O}^{S} - {rf}_{SS , {H}_{2} O} \cdot {\sum }_{( u , SS ) ∈ UC}^{ } {N}_{u , SS , s}^{S} = 0 \qquad ( 329 )$$

Set fraction of sour species in bottoms

N_{SS,SP}_{_{SS}}_{,s}^{S}−x_{SS,SP}_{_{SS}}_{,s}^{Kn}·N_{SS,SP}_{_{SS}}_{,s}^{T}=0∀(SS,SP_{SS},s)∈S^{UF} (330)

Energy balance using reboiler and condensor

Q_{SS}^{Reb}+Q_{SS}^{Cond}−Q_{SS}=0 (331)

Set energy use for reboiler and condensor

HR_{SS}·Q_{SS}^{Reb}+Q_{SS}^{Cond}=0 (332)

Biological digestor

Set biogas ratio of CH₄ to CO₂

N_{BD,CC,CH}_{₄}^{S}−cr_{BD}·N_{BD,CC,CO}_{₂}^{S}=0 (333)

Reverse osmosis

Set removal fraction of solids

N_{RO,SP}_{_{RO}}_{,s}^{S}−rf_{RO}·N_{MX}_{_{RO}}_{,RO,s}^{S}=0∀s∈S_{Sol} (334)

Cooling cycle

Cooling tower flow rate from energy requirement

Q_{C}−hr_{COOL-P}·N_{CLTR,COOL-P,H}_{₂}_{O}^{S}=0 (335)

Cooling tower evaporation loss

N_{CLTR}^{Evap}−0.00085·ΔT_{CLTR}·N_{CLTR,COOL-P,H}_{₂}_{O}^{S}=0 (336)

Cooling tower drift loss

N_{CLTR}^{Drift}−0.001·N_{MX}_{_{CLTR}}_{,CLTR,H}_{₂}_{O}^{S}=0 (337)

Sum total cooling tower losses

N_{CLTR}^{Evap}+N_{CLTR}^{Drift}−N_{CLTR,OUT}_{_{V}}_{,H}_{₂}_{O}^{S}=0 (338)

Set known cooling tower output solid concentrations

x_{CLTR,SP}_{_{CLTR}}_{,s}^{Kn}·N_{CLTR,SP}_{_{CLTR}}^{T}−N_{CLTR,SP}_{_{CLTR}}_{,s}^{S}=0∀s∈S_{Sol} (339)

Steam cycle

Set known process steam boiler output solid concentrations

x_{CLTR,SP}_{_{CLTR}}_{,s}^{Kn}·N_{CLTR,SP}_{_{CLTR}}^{T}−N_{CLTR,SP}_{_{CLTR}}_{,s}^{S}=0∀s∈S_{Sol} (340)

Set known heat engine boiler output solid concentrations

x_{X}_{_{PWB}}_{,MX}_{_{BLR}}_{,s}^{Kn}·N_{X}_{_{PWB}}_{,MX}_{_{BLR}}^{T}−N_{X}_{_{PWB}}_{,MX}_{_{BLR}}_{,s}^{S}=0∀s∈S_{Sol} (341)

Outlet wastewater

Upper bound on output wastewater concentrations

N_{MX}_{_{WW}}_{,OUT}_{_{V}}_{,s}^{S}−x_{MX}_{_{WW}}_{,OUT}_{_{V}}_{,s}^{Max}·N_{MX}_{_{WW}}_{,OUT}_{_{V}}^{T}≦0∀a∈S_{WW} (342)

Hydrogen/oxygen production
Pressure-swing adsorption

Set recovery fraction of H₂ from inlet

$${N}_{PSA , {SP}_{{H}_{2} p , {H}_{2}}}^{S} - {Rev}_{PSA}^{{H}_{2}} \cdot {\sum }_{( u , PSA ) ∈ UC}^{ } {N}_{u , PSA , {H}_{2}}^{S} = 0 \qquad ( 343 )$$

Set inlet mole fraction of H₂

$${\sum }_{( u , PSA ) ∈ UC}^{ } {N}_{u , PSA , {H}_{2}}^{S} - {In}_{PSA}^{{H}_{2}} \cdot {\sum }_{( u , PSA ) ∈ UC}^{ } {N}_{u , PSA}^{S} = 0 \qquad ( 344 )$$

Air separation unit

Recovery fraction of O₂

N_{ASU,OUT}_{_{V}}_{,s}^{S}−(1−sf_{ASU})·N_{AC,ASU,s}^{S}=0∀s∈S_{ASU}^{U} (345)

Process hot/cold/power utility requirements

Set electricity needed for process units

$${Q}_{P}^{El} - {\sum }_{u ∈ {U}_{Utill}}^{ } {S}_{u} \cdot {El}_{u}^{Base} = 0 \qquad ( 346 )$$

Set cooling water needed for process units

$${Q}_{P}^{CW} - {\sum }_{u ∈ {U}_{Utill}}^{ } {S}_{u} \cdot {CW}_{u}^{Base} = 0 \qquad ( 347 )$$

Set heating fuel needed for process units

$${Q}_{FCM} - {\sum }_{u ∈ {U}_{Utill}}^{ } {S}_{u} \cdot {F}_{u}^{Base} = 0 \qquad ( 348 )$$

Set utilities needed for process units

Q_{u,ut}^{HU}−Sᵤ·U_{u,ut}^{Base}=0∀ut,u∈U_{Util} (349)

Process costs
Feedstock costs

Levelized cost of biomass feedstock

$${Cost}_{s}^{F} = \frac{{MW}_{s} \cdot {N}_{{IN}_{BIO} , BDR , s}^{S} \cdot {C}_{s}^{F}}{Prod \cdot {LHV}_{Prod}} \\ ∀ s ∈ {S}_{Bio} \qquad ( 350 )$$

Levelized cost of coal feedstock

$${Cost}_{s}^{F} = \frac{{MW}_{s} \cdot {N}_{{IN}_{COAL} , CDR , s}^{S} \cdot {C}_{s}^{F}}{Prod \cdot {LHV}_{Prod}} ∀ s ∈ {S}_{Coal} \qquad ( 351 )$$

Levelized cost of natural gas feedstock

$${Cost}_{s}^{F} = {\sum }_{( {IN}_{NG} , u ) ∈ UC} \frac{{MW}_{s} \cdot {N}_{{IN}_{NG} , u , s}^{S} \cdot {C}_{s}^{F}}{Prod \cdot {LHV}_{Prod}} ∀ s ∈ {S}_{NG} \qquad ( 352 )$$

Levelized cost of freshwater feedstock

$${Cost}_{{H}_{2} O}^{F} = \frac{{MW}_{{H}_{2} O} \cdot {N}_{{IN}_{{H}_{2} O} , {SP}_{WRI} , {H}_{2} O}^{S} \cdot {C}_{{H}_{2} O}^{F}}{Prod \cdot {LHV}_{Prod}} \qquad ( 353 )$$

Electricity costs

Levelized cost of electricity

$${Cost}^{EI} = \frac{{F}_{In}^{EI} \cdot {C}_{In}^{EI} - {F}_{Out}^{EI} \cdot {C}_{Out}^{EI}}{Prod \cdot {LHV}_{Prod}} \qquad ( 354 )$$

CO₂ sequestration costs

Levelized cost of CO₂ sequestration

$${Cost}^{Seq} = \frac{{MW}_{{CO}_{2}} \cdot {N}_{{CO}_{2} , SC , {OUT}_{{CO}_{2}} , {CO}_{2}}^{S} \cdot {C}^{Seq}}{Prod \cdot {LHV}_{Prod}} \qquad ( 355 )$$

Levelized investment costs

Total overnight cost of process units

$${TOC}_{u} = ( 1 + {IC}_{u} ) \cdot ( 1 + {BOP}_{u} ) \cdot {C}_{o , u} \cdot \frac{{S}_{u}^{{sf}_{u}}}{{S}_{o , u}} \qquad ( 356 )$$

Variable capital costs of process units

CCᵤ=LCCR·IDCF·TOCᵤ (357)

Levelized cost of process units

$${Cost}_{u}^{U} = \frac{{CC}_{u} \cdot ( 1 + OM )}{CAP \cdot Prod \cdot {LHV}_{Prod}} \qquad ( 358 )$$

Objective function

Levelized cost of fuel production

$$MIN {\sum }_{u ∈ U} {\sum }_{In ( u , s ) ∈ {S}^{U}} {Cost}_{s}^{F} + {Cost}^{EI} + {Cost}^{Seq} + {\sum }_{u ∈ {U}_{Inv}} {Cost}_{u}^{U} \qquad ( 359 )$$

Simultaneous heat and power integration
Pinch points

Set pinch points based on inlet temperatures

$$\{ \begin{matrix}{T}_{pi} = {T}_{u , {u}^{′}}^{HP - in} & ∀ ( u , {u}^{′} ) ∈ HP ; & {T}_{pi} = Tu & ∀ u ∈ {HPt}^{HB} ; & \\ {T}_{pi} = {T}_{ut} & ∀ ( ut , pi ) ∈ HPt - {PI}^{Ut} ; & {T}_{pi} = {T}_{b , c , t}^{PC - in} & \begin{matrix}∀ ( b , c , t ) ∈ \\ HEP\end{matrix} ; & {T}_{pi} = {T}_{c} \\ {T}_{pi} = {T}_{u , {u}^{′}}^{CP - in} + Δ T & ∀ ( u , {u}^{′} ) ∈ CP ; & {T}_{pi} = {T}_{b , c}^{EC - in} + Δ T & ∀ ( b , c ) ∈ {CP}^{EC} ; & \\ {T}_{pi} = {T}_{b , t}^{SH - in} + Δ T & ∀ ( b , t ) ∈ {CP}^{SH} ; & {T}_{pi} = {T}_{ut} + Δ T & \begin{matrix}∀ ( ut , pi ) ∈ \\ CPt - {PI}^{Ut}\end{matrix} ; & \\ {T}_{pi} = {T}_{b} + Δ T & & & & \end{matrix} \} \qquad ( 360 )$$

Temperature differences

Process unit hot stream inlets

ΔT_{u,u′pi}^{HP-in}=max{0,T_{u,u′}^{HP-in}−Tₚᵢ} (361)

Process unit hot stream outlets

ΔT_{u,u′pi}^{HP-out}=max{0,T_{u,u′}^{HP-out}−Tₚᵢ} (362)

Process unit cold stream inlets

ΔT_{u,u′pi}^{CP-in}=max{0,T_{u,u′}^{CP-in}−(Tₚᵢ−ΔT)} (363)

Process unit cold stream outlets

ΔT_{u,u′pi}^{CP-out}=max{0,T_{u,u′}^{CP-out}−(Tₚᵢ−ΔT)} (364)

Heat engine precooler inlets

ΔT_{b,c,t,pi}^{PC-in}=max{0,T_{b,c,t}^{PC-in}−Tₚᵢ} (365)

Heat engine precooler outlets

ΔT_{b,c,t,pi}^{PC-out}=max{0,T_{b,c,t}^{PC-out}−Tₚᵢ} (366)

Heat engine economizer inlets

ΔT_{b,c,pi}^{EC-in}=max{0,T_{b,c}^{EC-in}−(Tₚᵢ−ΔT)} (367)

Heat engine economizer outlets

ΔT_{b,c,pi}^{EC-out}=max{0,T_{b,c}^{EC-out}−(Tₚᵢ−ΔT)} (368)

Heat engine superheater inlets

ΔT_{b,t,pi}^{SH-in}=max{0,T_{b,t}^{SH-in}−(Tₚᵢ−ΔT)} (369)

Heat engine superheater outlets

ΔT_{b,t,pi}^{SH-out}=max{0,T_{b,t}^{SH-out}−(Tₚᵢ−ΔT)} (370)

Heat engine logical existence

Bound on heat engine flow rate

F_{b,c,t}^{Up}·y_{b,c,t}^{En}≧F_{b,c,t}^{En}∀(b,c,t)∈HEP (371)

Bound on total amount of heat engines

$${\sum }_{( b , c , t ) ∈ HEP} {y}_{b , c , t}^{En} \leq En Max \qquad ( 372 )$$

Heat balances

Heat engine electricity balance

$${\sum }_{( b , c , t ) ∈ HEP} ( {w}_{b , c , t}^{Tur} - {w}_{b , c , t}^{Pum} ) \cdot {F}_{b , c , t}^{En} = {F}_{El} \qquad ( 373 )$$

Upper heat balance for pinch points

$${Q}_{pi}^{H} = {\sum }_{( u , {u}^{′} ) ∈ HP} {\sum }_{s} {N}_{u , {u}^{′} , s}^{s} \cdot {Cp}_{u , {u}^{′} , s}^{P} \cdot ( Δ {T}_{u , {u}^{′} , pi}^{HP - in} - Δ {T}_{u , {u}^{′} , pi}^{HP - out} ) + {\sum }_{( b , c , t ) ∈ HEP} {F}_{b , c , t}^{En} \cdot {Cp}^{HE - P} \cdot ( Δ {T}_{b , c , t , pi}^{PC - in} - Δ {T}_{b , c , t , pi}^{PC - out} ) + {\sum }_{( ut , pi ) ∈ HPt - {PI}^{Ut}}^{Ut} {\sum }_{( u , ut ) ∈ HPt} {Q}_{u , ut}^{HU} + {\sum }_{( u , pi ) ∈ HPt - {PI}^{HB}} {Q}_{u} + {\sum }_{b} {\sum }_{( c , pi ) ∈ HPt - {PI}^{C}} {\sum }_{t} {F}_{b , c , t}^{En} \cdot {dH}_{C}^{C} \qquad ( 374 )$$

Lower heat balance for pinch points

$${Q}_{pi}^{C} = {\sum }_{( u , {u}^{′} ) ∈ CP} {\sum }_{s} {N}_{u , {u}^{′} , s}^{s} \cdot {Cp}_{u , {u}^{′} , s}^{P} \cdot ( Δ {T}_{u , {u}^{′} , pi}^{CP - out} - Δ {T}_{u , {u}^{′} , pi}^{CP - in} ) + {\sum }_{( b , c , t ) ∈ HEP} {F}_{b , c , t}^{En} \cdot {Cp}^{HE - E} \cdot ( Δ {T}_{b , c , t , pi}^{EC - out} - Δ {T}_{b , c , pi}^{EC - in} ) + {\sum }_{( b , c , t ) ∈ HEP} {F}_{b , c , t}^{En} \cdot {Cp}^{HE - S} \cdot ( Δ {T}_{b , t , pi}^{SH - out} - Δ {T}_{b , t , pi}^{SH - in} ) + {\sum }_{( ut , pi ) ∈ CPt - {PI}^{Ut}} {\sum }_{( u , ut ) ∈ CPt} {Q}_{u , ut}^{HU} + {\sum }_{( b , pi ) ∈ CPt - {PI}^{B}} {\sum }_{c} {\sum }_{t} {F}_{b , c , t}^{En} \cdot {dH}_{b}^{B} \qquad ( 375 )$$

Pinch point heating deficit

zₚᵢ=Qₚᵢ^{C}−Qₚᵢ^{H} (376)

Negativity of pinch deficits

zₚᵢ≦0 (377)

Total heating deficit

Ω−Q_{c}=0 (378)

Total heat balance

$$Ω = {\sum }_{( u , {u}^{′} ) ∈ HP} {\sum }_{s} {N}_{u , {u}^{′} , s}^{s} \cdot {Cp}_{u , {u}^{′} , s}^{P} \cdot ( {T}_{u , {u}^{′}}^{HP - in} - {T}_{u , {u}^{′}}^{HP - out} ) + {\sum }_{( b , c , t ) ∈ HEP} {F}_{b , c , t}^{En} \cdot {Cp}^{HE - P} \cdot ( {T}_{b , c , t}^{PC - in} - {T}_{b , c , t}^{PC - out} ) + {\sum }_{( u , ut ) ∈ HPt} {Q}_{u , ut}^{HU} + {\sum }_{u ∈ {HPt}^{HB}} {Q}_{u} + {\sum }_{( b , c , t ) ∈ HEP} {F}_{b , c , t}^{En} \cdot {dH}_{c}^{C} - {\sum }_{( u , {u}^{′} ) ∈ CP} {\sum }_{s} {N}_{u , {u}^{′} , s}^{s} \cdot {Cp}_{u , {u}^{′} , s}^{P} \cdot ( {T}_{u , {u}^{′}}^{CP - out} - {T}_{u , {u}^{′}}^{CP - in} ) - {\sum }_{( b , c , t ) ∈ HEP} {F}_{b , c , t}^{En} \cdot {Cp}^{HE - E} \cdot ( {T}_{b , c}^{EC - out} - {T}_{b , c}^{EC - in} ) - {\sum }_{( b , c , t ) ∈ HEP} {F}_{b , c , t}^{En} \cdot {Cp}^{HE - S} \cdot ( {T}_{b , t}^{SH - out} - {T}_{b , t}^{SH - in} ) - {\sum }_{( u , ut ) ∈ CPt} {Q}_{u , ut}^{HU} - {\sum }_{( b , c , t ) ∈ HEP} {F}_{b , c , t}^{En} \cdot {dH}_{b}^{B} \qquad ( 379 )$$

### Example 3.17

### CBGTL Process Superstructure Conceptual Design

The syngas conversion and hydrocarbon upgrading units proposed herein are based on an extension of the CBGTL refinery superstructure in Examples 1 and 2. The flowsheets depicting the complete superstructure are shown in FIGS. 38-50. In the figures, fixed process units are represented by 110, variable process units by 120, splitter units by 130, and mixer units by 140. The variable process streams are represented by 210 and all other process streams are fixed, unless otherwise indicated. The CBGTL superstructure is designed to co-feed biomass, coal, or natural gas to produce gasoline, diesel, and kerosene. Syngas is generated via gasification from biomass (FIG. 38) or coal (FIG. 39) or auto-thermal reaction of natural gas (FIG. 50) and is either (i) converted into hydrocarbon products in the Fischer-Tropsch (FT) reactors (FIG. 42) or (ii) into methanol via methanol synthesis (FIG. 45). The FT wax will be sent to a hydrocracker to produce distillate and naphtha (FIG. 44) while the FT vapor effluent may be (a) fractionated and upgraded into gasoline, diesel, or kerosene or (FIG. 44) (b) catalytically converted to gasoline via a ZSM-5 zeolite (FIG. 43). The methanol may be either (a) catalytically converted to gasoline via the ZSM-5 catalyst (FIGS. 45-46) or (b) catalytically converted to olefins via the ZSM-5 catalyst and subsequently fractionated to distillate and gasoline (FIG. 45).

Acid gases including CO₂, H₂, and NH₃ are removed from the syngas via a Rectisol unit prior to conversion to hydrocarbons or methanol (FIG. 40). Incorporation of other acid gas removal technologies (e.g., amine adsorption, pressure-swing adsorption, vacuum-swing adsorption, membrane separation) and their relative capital/operating cost as a function of input flow rate and acid gas concentration is the subject of an ongoing study. The sulfur-rich gases are directed to a Claus recovery process (FIG. 41) and the recovered CO₂ may be sequestered (FIG. 40) or reacted with H₂ via the reverse water-gas-shift reaction. The CO₂ may be directed to either the gasifiers (FIGS. 38-39), the reverse water-gas-shift reactor (FIG. 40), or the iron-based FT units (FIG. 42). Recovered CO₂ is not sent to the cobalt-based FT units to ensure a maximum molar concentration of 3% and prevent poisoning of the catalyst. Hydrogen is produced via pressure-swing adsorption or an electrolyzer unit while oxygen can be provided by the electrolyzer or a separate air separation unit (FIG. 48). A complete water treatment network (FIGS. 49-50) is incorporated that will treat and recycle wastewater from various process units, blowdown from the cooling tower, blowdown from the boilers, and input freshwater. Clean output of the network includes (i) process water to the electrolyzers, (ii) steam to the gasifiers, auto-thermal reactor, and water-gas-shift reactor, and (iii) discharged wastewater to the environment.

### Example 3.18

### Fischer-Tropsch Synthesis

The four FT units considered in Examples 1 and 2 utilized either a cobalt or iron catalyst and operated at high or low temperature. The two cobalt-based FT units would not facilitate the water-gas-shift reaction and therefore required a minimal level of CO₂ input to the units. The two iron-based FT units were assumed to facilitate the reverse water-gas-shift reaction and therefore could consume CO₂ within the unit using H₂ to produce the CO necessary for the FT reactions. A key consequence of the reaction conditions in the latter units was the heat needed for the reverse water-gas-shift reaction would be provided by the highly exothermic FT reaction. In this study, the set of possible FT units is expanded to consider iron-based systems that will facilitate the forward water-gas-shift reaction within the units. These FT units will require a lower H₂/CO ratio for the FT reaction because steam in the feed will be shifted to H₂ through consumption of CO. These units may be beneficial since certain syngas generation units (e.g., coal gasifiers) will produce a gas that generally has a H₂/CO ratio that is much less than the 2/1 requirement for FT synthesis (Baliban et al., 2010 and Kreutz et al., 2008, which are incorporated herein by reference as if fully set forth). The downside of the new FT units will be the high quantity of CO₂ that is produced as a result of the water-gas-shift reaction. The framework developed for the CBGTL superstructure will directly examine the benefits and consequences for each of the six FT units to determine which technology produces a refinery with a superior design. The mathematical model will select at most two units to operate in the final process design.

FIG. 42 shows the flowsheet for FT hydrocarbon production within the superstructure. Clean gas from the acid gas removal (AGR) unit is mixed with recycle light gases from a CO₂ separator (CO₂SEP) and split (SP_{CG}) to either the low-wax FT section (SP_{FTM}), the nominal-wax FT section (SP_{FTN}), or methanol synthesis (MEOHS). Each FT section will have three distinct FT units based on the operating conditions of the unit. The cobalt-based FT units operate at either low temperature (LTFT; 240° C.) or high temperature (HTFT; 320° C.) and must have a minimal amount of CO₂ in the input stream. Two iron-based FT units will facilitate the reverse water-gas-shift (rWGS)reaction and will operate at low (LTFTRGS; 240° C.) and high temperature (HTFTRGS; 320° C.). The other two iron-based FT units will use the forward reverse water-gas-shift (fWGS) units, operate at a mid-level temperature (267° C.), and produce either minimal (MTFTWGS-M) or nominal (MTFTWGS-N) amounts of wax.

Hydrogen may be recycled to any of the FT units to either shift the H₂/CO ratio or the H₂/CO₂ ratio to the appropriate level. Steam may alternatively be used as a feed for the two iron-based fWGS FT units to shift the H₂/CO ratio. CO₂ may be recycled back to the iron-based rWGS FT units to be consumed in the WGS reaction. Similarly, the pressure-swing adsorption (PSA) offgas which will be lean in H₂ may be recycled to the iron-based rWGS FT units for consumption of the CO or CO₂. The effluent from the auto-thermal reactor (ATR) will contain a H₂/CO ratio that is generally above 2/1, and is therefore favorable as a feedstock for FT synthesis [5]. However, the concentration of CO₂ within the ATR effluent will prevent the stream from being fed to the cobalt-based units. The two streams exiting the FT units will be a waxy liquid phase and a vapor phase containing a range of hydrocarbons. The wax will be directed to a hydrocracker (WHC) while the vapor phase is split (SP_{FTH}) for further processing.

### Example 3.19

### Fischer-Tropsch Product Upgrading

The vapor phase effluent from FT synthesis will contain a mixture of C₁-C₃₀₊ hydrocarbons, water, and some oxygenated species. FIG. 43 details the process flowsheet used to process this effluent stream. The stream will be split (SP_{FTH}) and can pass through a series of treatment units designed to cool the stream and knock out the water and oxygenates for treatment. Initially, the watersoluble oxygenates are stripped (WSOS) from the stream. The stream is then passed to a three-phase separator (VLWS) to remove the aqueous phase from the residual vapor and any hydrocarbon liquid. Any oxygenates that are present in the vapor phase may be removed using an additional separation unit (VSOS). The water lean FT hydrocarbons are then sent to a hydrocarbon recovery column for fractionation and further processing (FIG. 44). The oxygenates and water removed from the stream are mixed (MX_{FTWW}) and sent to the sour stripper mixer (MXSS) for treatment.

The FT hydrocarbons split from SPFTH may also be passed over a ZSM-5 catalytic reactor (FT-ZSM5) to be converted into mostly gasoline range hydrocarbons and some distillate. The ZSM-5 unit will be able to convert the oxygenates to additional hydrocarbons, so no separate processing of the oxygenates will be required for the aqueous effluent. The raw product from FT-ZSM5 is fractionated (ZSM5F) to separate the water and distillate from the gasoline product. The water is mixed with other wastewater knockout (MXPUWW) and the distillate is hydrotreated (DHT) to form a diesel product. The raw ZSM-5 HC product is sent to the LPG-gasoline separation section for further processing (FIG. 46).

The water lean FT hydrocarbons leaving MX_{FTWW} are sent to a hydrocarbon recovery column (HRC), as shown in FIG. 44. The hydrocarbons are split into C₃-C₅ gases, naphtha, kerosene, distillate, wax, offgas, and wastewater [Bechtel, 1992 and Baliban et al., 2010, which are incorporated herein by reference as if fully set forth). The upgrading of each stream will follow a detailed Bechtel design (Bechtel, 1992, which is incorporated herein by reference as if fully set forth) which includes a wax hydrocracker (WHC), a distillate hydrotreater (DHT), a kerosene hydrotreater (KHT), a naphtha hydrotreater (NHT), a naphtha reformer (NRF), a C₄ isomerizer (C₄I), a C₅/C₆ isomerizer (C₅₆I), a C₃/C₄/C₅ alkylation unit (C₃₄₅A), and a saturated gas plant (SGP).

The kerosene and distillate cuts are hydrotreated in (KHT) and (DHT), respectively, to remove sour water and form the products kerosene and diesel. Any additional distillate or kerosene produced in other sections of the refinery will also be directed to these units for processing. The naphtha cut is sent to a hydrotreater (NHT) to remove sour water and separate C₅-C₆ gases from the treated naphtha. The wax cut is sent to a hydrocracker (WHC) where finished diesel product is sent to the diesel blender (DBL) along with the diesel product from (DHT). C₅-C₆ gases from (NHT) and (WHC) are sent to an isomerizer (C₅₆). Hydrotreated naphtha is sent to the naphtha reformer (NRF). The C₄ isomerizer (C₄I) converts in-plant and purchased butane to isobutane, which is fed into the alkylation unit (C₃₄₅A). Purchased butane is added to the isomerizer such that 80 wt % of the total flow entering the unit is composed of n-butane. Isomerized C₄ gases are mixed with the C₃-C₅ gases from the (HRC) in (C₃₄₅A), where the C₃-C₅ olefins are converted to high-octane gasoline blending stock. The remaining butane is sent back to (C₄I), while all light gases are mixed with the offgases from other unit and sent to the saturated gas plant (SGP). C₄ gases from (SGP) are recycled back to the (C₄I) and a cut of the C₃ gases are sold as byproduct propane.

### Example 3.20

### Methanol Synthesis and Conversion

The clean gas split (SP_{CG}) from the acid gas recovery unit may be directed to a methanol synthesis unit (MEOHS) for conversion of the syngas to methanol. The flowsheet for the production and subsequent conversion of methanol is shown in FIG. 45. The syngas entering MEOHS may be combined with recycle hydrogen to increase the H₂/CO ratio to the desired 2/1 level for synthesis. The raw methanol product is directed to a degasser (MEDEG) to remove any unreacted syngas which is recycled back to the process (SP_{LG}). The purified methanol is split (SP_{MEOH}) into one of two major conversion pathways including methanol to gasoline (MTG) and methanol to olefins (MTO). The MTG process will utilize the ZSM-5 zeolite to produce gasoline range hydrocarbons which are directed to the LPG-gasoline separation section (FIG. 46). The MTO process also uses the ZSM-5 zeolite to produce a range of olefins which can be upgraded into a mixture of gasoline and distillate within an oligomerization reactor (MOGD). The ratio of gasoline to distillate will vary depending on the operating conditions in the MTO and MOGD reactors. The raw MOGD product is then fractionated to produce a distillate cut, a kerosene cut, and a gasoline cut which are directed to the DHT unit, the KHT unit, and the LPG-gasoline separation section, respectively. The operational ratio of kerosene to total distillate reported in the literature for the MTOD process is about 30%, though this number may be increased by tailoring the operating conditions within the MTO and MOGD units to yield the appropriate range of hydrocarbons.

### Example 3.21

### LPG-Gasoline Separation

The gasoline range hydrocarbons produced by the FT-ZSM5 unit, the MTG unit, or the MTOD process must be sent to the LPG-gasoline separation flowsheet depicted in FIG. 46. Each hydrocarbon stream is split (SP_{FTZSM}, SP_{MTGHC}, and SP_{MTODHC}, respectively) and sent to a hydrocarbon knockout unit for light gas removal. The first knock-out unit (HCKO1) will not incorporate additional CO₂ separation, so the CO₂ rich light gases recovered from HCKO1 will be recycled back to the process (SP_{LG}). The second knock-out unit (HCKO2) will separate out CO₂ from the recovered light gases for sequestration or recycle back to additional process units (MX_{CO2C}). The CO₂ lean light gases will be recycled back to the process.

The crude liquid hydrocarbons recovered from the two knock-out units is sent to a deethanizer (DEETH) to remove any C₁—C hydrocarbons. The light HC gases are sent to an absorber column (ABS-COL) where a lean oil recycle is used to strip the C₃₊ HCs from the input. The liquid bottoms from the ABS-COL is then refluxed back to the deethanizer. The C₃₊ HCs from the bottom of the deethanizer are sent to a stabilizer column (STA-COL) where the C₃/C₄ hydrocarbons are removed and alkylated (ALK-UN) to produce iso-octane and an LPG byproduct. Additional isobutane (INBUT) may be fed to the alkylation unit for increased alkylate production. The bottoms from the stabilizer column is sent to a splitter column (SP-COL) to recover a lean oil recycle from the column top for use in the absorber column. Light and heavy gasoline fractions are recovered from the column top and bottom, respectively. The LPG/alkylate from the alkylation unit is split (LPG-ALK) into an LPG byproduct (OUT_{LPG}) and an alkylate fraction which is blended with the gasoline fractions from the splitter column (OUT_{GAS}).

### Example 3.22

### Mathematical Model for Process Synthesis with Simultaneous Heat, Power, and Water Integration

This example will discuss the enhancements to the previous mathematical model for process synthesis and simultaneous heat, power, and water integration that will incorporate a wide variety of designs for syngas conversion and hydrocarbon upgrading. Modeling of these enhancements will be described in detail in the following section and the complete mathematical model is listed in Example 3.15.

### NOMENCLATURE

The nomenclature used in the mathematical description below is outlined in Table 45. Note that this table represents a subset of the comprehensive list of symbols that are needed for the full mathematical model. The full list of symbols and mathematical model are included for reference in Example 3.15.

TABLE 45
Mathematical model nomenclature

| | | | |
|---|---|---|---|
| Symbol | Definition | Symbol | Definition |

Indices

| | | | |
|---|---|---|---|
| s | Species index | u | Process unit index |
| r | Reaction index | a | Atom index |

Sets

| | | | |
|---|---|---|---|
| (u,u′) ∈ UC | Set of all streams from unit u to unit u′ | (u,u′,s) ∈ S^{UF} | Set of all species s within stream (u,u′) |
| s ∈ Sᵤ^{U} | Set of all species s existing within unit u | a ∈ Aₐ^{U} | Set of all atoms a existing within unit u |
| u ∈ U_{Sp}^{Bal} | Set of all units u using a species balance | u ∈ U_{Ar}^{Bal} | Set of all units u using an atom balance |
| (u,r,s) ∈ R^{U} | Set for the key species s of reaction r in unit u | u ∈ U_{irFT-RGS} | Set of iron-based FT units with rWGS reaction |
| u ∈ U_{CoFT} | Set of cobalt-based FT units | | |
| u ∈ U_{IrFT-WGS} | Set of iron-based FT units with tWGS reaction | | |

Parameters

| | | | |
|---|---|---|---|
| AR_{s,a} | Atomic ratio of atom a in species s | v_{r,s} | Coefficient for species s in reaction r |
| fcᵣ^{u} | Conversion of key species of reaction r in unit u | H_{u,u′,s}^{S} | Specific enthalpy of species s in stream (u,u′) |
| FTR_{u,H}₂—H₂O | Ratio of H₂/H₂O needed for FT unit u | FTR_{u,H}₂—CO | Ratio of H₂/CO needed for FT unit u |
| FTR_{u,H}₂—CO₂ | Ratio of H₂/CO₂ needed for FT unit u | Wₙ | Mass fraction of Cₙ hydrocarbons after FT reaction |
| α | Chain growth parameter for FT reaction | cfₙ | Carbon fraction present in Cₙ hydrocarbons after |
| | | | FT reaction |

Variables

| | | | |
|---|---|---|---|
| N_{a,a′,s}^{S} | Molar flow of species s from unit a to unit a′ | ξᵣ^{u} | Extent of conversion of reaction r in unit u |
| H_{u,u′}^{T} | Total enthalpy of stream (u,u′) | Qᵤ | Heat transfer to/from unit u |
| Qᵤ^{L} | Heat loss from unit u | Wᵤ | Work need for/required by unit u |
| yᵤ | Logical existence of unit u | | |

Heat & Mass Flows

Mass flow for all species is constrained by either a species balance (Eqn. 1/Eqn. 2 of Baliban et al., 2011, which is incorporated herein by reference as if fully set forth, or an atom balance (Eqn. 3/Eqn. 3 of Baliban et al., 2011, which is incorporated herein by reference as if fully set forth. The units requiring a species balance, U_{Sp}^{Bal}, will include the mixer units, the splitter units, and the flash units. The remainder of the units detailed in the above five figures will require an atom balance, U_{At}^{Bal}. The species balance is used for all units that are governed by a set of reactions ((u, r, s)∈R^{U}) with known extents of conversion (86 ᵣⁿ) of a key species (Eqn. 381).

$$\begin{matrix}{\sum }_{( {u}^{′} , u ) ∈ UC} {N}_{{u}^{′} , u , s}^{S} - {\sum }_{( u , r , {s}^{′} ) ∈ {R}^{U}} \frac{{V}_{r , s}}{{V}_{r , {s}^{′}}} \cdot {ξ}_{r}^{u} - {\sum }_{( u , {u}^{′} ) ∈ UC} {N}_{u , {u}^{′} , s}^{S} = 0 \\ ∀ s ∈ {S}_{u}^{U} , u ∈ {U}_{Sp}^{Bal} & ( 380 ) \\ {ξ}_{r}^{u} - {fc}_{r}^{u} \cdot {\sum }_{( {u}^{′} , u , s ) ∈ {S}^{UF}} {N}_{{u}^{′} , u , s}^{S} = 0 \\ ∀ ( u , r , s ) ∈ {R}^{U} & ( 381 ) \\ {\sum }_{( {u}^{′} , u , s ) ∈ {s}^{UF}} {AR}_{s , a} \cdot {N}_{{u}^{′} , u , s}^{S} - {\sum }_{( u , {u}^{′} , s ) ∈ {S}^{UF}} {AR}_{s , a} \cdot {N}_{u , {u}^{′} , s}^{S} = 0 \\ ∀ a ∈ {A}_{u}^{U} , u ∈ {U}_{At}^{Bal} & ( 382 )\end{matrix}$$

Heat balances across every unit are maintained using Equation 383 (Eqn. 12 of Baliban et al., 2011, which is incorporated herein by reference as if fully set forth). The relevant terms include the input and output stream enthalpies (H), the heat transferred to/from the unit (Q), the heat lost from the unit (Q^{L}), and the work done by the unit (W). Note that Equation 383 is a general equation for the entire CBGTL refinery, and some of the terms are not needed for each unit. Specifically, the heat loss across all units in the hydrocarbon production and upgrading section is negligible (Q_{L}=0). The total enthalpy of a stream is related to the enthalpy of the individual components through Equation 384 (Eqn. 13 of Baliban et al., 2011, which is incorporated herein by reference as if fully set forth) only for streams with known thermodynamic conditions. Each unit in the hydrocarbon production and upgrading section unit will operate at a known temperature and pressure, so the specific outlet enthalpies of each species, in these units can be determined a priori. Note that Equations 383 and 384 suffice to define the enthalpy flow throughout the entire system while leaving degrees of freedom for the heat transfer (Q) to/from the necessary process units.

$$\begin{matrix}{\sum }_{( u , {u}^{′} ) ∈ UC} {H}_{u , {u}^{′}}^{T} - {\sum }_{( {u}^{′} , u ) ∈ UC} {H}_{{u}^{′} , u}^{T} - {Q}_{u} - {Q}_{u}^{L} - {W}_{u} = 0 \\ ∀ u ∈ U & ( 383 ) \\ {H}_{u , {u}^{′}}^{T} - {\sum }_{( u , {u}^{′} , s ) ∈ {S}^{UF}} {H}_{u , {u}^{′} , s}^{S} = 0 ∀ ( u , {u}^{′} ) ∈ UC & ( 384 )\end{matrix}$$

Fischer-Tropsch Units

The process superstructure will consider six different types of FT units. Two of the units (U_{CoFT}) will utilize a cobalt-based catalyst and four will use an iron-based catalyst (U_{IrFT}). For transportation fuel production, the hydrocarbons should have minimal oxygen formation (p˜0) and long chain lengths (2n˜m).

nCO+(n−p+0.5m)H₂→CₙHₘOₚ+(n−p)H₂O (385)

CO₂+H₂ [symbol] CO+H₂O (386)

This yields a H₂ to CO ratio of approximately 2 (FTR_{u,CO}=2). If the FT units utilize a cobalt based catalyst, then the reverse water-gas-shift reaction (Eqn. 386) will not occur, and the above ratio is appropriate for maximum production of hydrocarbons. If the FT units use an iron-based catalyst, then the reaction will occur within the units. If the forward water-gas-shift reaction is used, then hydrogen may be generated within the unit via reaction of H₂O with CO. Therefore, the input H₂ to CO ratio input to the unit may be less than the optimal requirement for FT synthesis (FTR_{u,CO}<2). If the reverse water-gas-shift reaction occurs within the unit, then enough hydrogen must be present to shift any CO₂ in tandem with a reaction of CO. Assuming a 2:1 ratio for the FT reaction, effectively 3 moles of H₂ will be needed to convert one mole of CO₂ to liquid products (FTR_{u,CO2}=3) since one mole of H₂ is needed for Equation 386 and 2 moles are needed for Equation 385. The appropriate ratio for the syngas entering an iron-based FT reactor should therefore be equal to 2 moles of H₂ per the molar sum of (CO+1.5CO₂). Equations 387-389 constrain the proper input ratios for H₂, CO, CO₂, and H₂O. Due to the use of the water-gas-shift reaction in the iron-based units, several light gas streams can also be directed to these units. The effluent stream from the auto-thermal reactor and the offgas from the pressure-swing adsorption column are split and may be partially sent to all four iron-based FT units. Preheated CO₂ and preheated H₂ can also be input to the two iron-based reverse WGS units while preheated steam can be input to the two iron-based forward WGS units.

$$\begin{matrix} {\sum }_{( {u}^{′} , u , {H}_{2} ) ∈ {S}^{UF}} {N}_{{u}^{′} , u , {H}_{2}}^{S} = {FTR}_{u , {H}_{2} - CO} \cdot {\sum }_{( {u}^{′} , u , CO ) ∈ {S}^{UF}} {N}_{{u}^{′} , u , CO}^{S} \\ ∀ u ∈ {U}_{CoFT} & ( 387 ) \\ {\sum }_{( {u}^{′} , u , {H}_{2} ) ∈ {S}^{UF}} {N}_{{u}^{′} , u , {H}_{2}}^{S} = {FTR}_{u , {H}_{2} - CO} \cdot {\sum }_{( {u}^{′} , u , CO ) ∈ {S}^{UF}} {N}_{{u}^{′} , u , CO}^{S} + {FTR}_{u , {H}_{2} - {CO}_{2}} \cdot {\sum }_{( {u}^{′} , u , {CO}_{2} ) ∈ {S}^{UF}} {N}_{{u}^{′} , u , {CO}_{2}}^{S} \\ ∀ u ∈ {U}_{IrFT - RGS} & ( 388 ) \\ {\sum }_{( {u}^{′} , u , {H}_{2} ) ∈ {S}^{UF}} {N}_{{u}^{′} , u , {H}_{2}}^{S} = {FTR}_{u , {H}_{2} - CO} \cdot {\sum }_{( {u}^{′} , u , CO ) ∈ {S}^{UF}} {N}_{{u}^{′} , u , CO}^{S} - {FTR}_{u , {H}_{2} - {H}_{2} O} \cdot {\sum }_{( {u}^{′} , u , {H}_{2} O ) ∈ {S}^{UF}} {N}_{{u}^{′} , u , {H}_{2} O}^{S} \\ ∀ u ∈ {U}_{IrFT - WGS} & ( 389 )\end{matrix}$$

The iron-based rWGS and cobalt-based FT units are modeled with stoichiometric reactions with known extents of reaction for each hydrocarbon in the effluent stream (Eqn. 381). C₁-C₂₀ paraffin and olefin hydrocarbons are modeled directly, while C₂₁-C₂₉ hydrocarbons are represented by pseudocomponents having properties consistent with 70 mol % olefin and 30 mol % paraffin. All C₃₀₊ compounds are represented by a generic wax pseudocomponent (C_{52.524}H_{105.648}O_{0.335}) (Bechtel, 1998, which is incorporated herein by reference as if fully set forth). Oxygenated compounds formed in the reactors are represented by vapor phase (C_{2.43}H_{5.69}O), aqueous phase (C_{1.95}H_{5.77}O_{1.92}), and organic phase (C_{4.78}H_{11.14}O_{1.1}) pseudocomponents. The total converted carbon present in each pseudocomponent is 0.1%, 1.0%, and 0.4%, respectively (Bechtel, 1998, which is incorporated herein by reference as if fully set forth).

2.43·CO+4.275·H₂→C_{2.43}H_{5.69}O+1.43·H₂O

1.95·CO+3.815·H₂→C_{1.95}H_{5.77}O_{1.02}+1.93·H₂O

4.78·CO+9.25·H₂→C_{4.78}H_{11.14}O_{1.1}+3.68·H₂O

All other hydrocarbon products up to C29 are represented by paraffin and olefin (one double bond) compounds, where the fraction of carbon in the paraffin form is 20% for C₂-C₄, 25% for C₅-C₆, and 30% for C₇-C₂₀ (Bechtel, 1998, which is incorporated herein by reference as if fully set forth). C₄-C₆ hydrocarbons are present in both linear and branched form with a branched carbon fraction of 5% for C₄ and 10% for C₅-C₆ (Bechtel, 1998, which is incorporated herein by reference as if fully set forth). C₂₁-C₂₉ hydrocarbons are represented by pseudocomponents having properties consistent with 70 mol % olefin and 30 mol % paraffin. All C₃₀₊ compounds are represented by a generic wax pseudocomponent (C_{52.524}H_{105.648}O_{0.335}) (Bechtel, 1998, which is incorporated herein by reference as if fully set forth).

The distribution of the hydrocarbon products can be assumed to follow the theoretical Anderson-Schulz-Flory (ASF) distribution based on the chain growth probability values (Eqn. 390),

Wₙ=n·(1−α)²·αⁿ⁻¹ (390)

where Wn is the mass fraction of the species with carbon number n and a is the chain growth probability. The high-temperature (320° C.) process has a lower chain growth probability (α=0.65) that favors the formation of gasoline-length hydrocarbons, while the low-temperature process (240° C.; α=0.73) forms heavier hydrocarbons and waxes (Dry, 2002, which is incorporated herein by reference as if fully set forth). To account for observed yields of the lighter hydrocarbons that are higher than what the ASF distribution predicts (Zwart and Boerrigter, 2005; Oukaci, 2002, which are incorporated herein by reference as if fully set forth), a slightly modified formula is used for the C₁-C₄ hydrocarbons (Eqns. 391-396).

$$\begin{matrix}{W}_{1} = \frac{1}{2} ( 1 - {\sum }_{n = 5}^{∞} {W}_{n} ) & ( 391 ) \\ {W}_{2} = \frac{1}{6} ( 1 - {\sum }_{n = 5}^{∞} {W}_{n} ) & ( 392 ) \\ {W}_{3} = \frac{1}{6} ( 1 - {\sum }_{n = 5}^{∞} {W}_{n} ) & ( 393 ) \\ {W}_{4} = \frac{1}{6} ( 1 - {\sum }_{n = 5}^{∞} {W}_{n} ) & ( 394 ) \\ {W}_{n} = {n ( 1 - α )}^{2} {α}^{n - 1} \\ ∀ 5 \leq n \leq 29 & ( 395 ) \\ {W}_{Wax} = {\sum }_{n = 30}^{∞} {n ( 1 - α )}^{2} {α}^{n - 1} & ( 396 )\end{matrix}$$

Given the weight fractions, the fraction of carbon present at each hydrocarbon length, crₙ, is defined in Equation 397. The overall conversion of carbon in each reactor is assumed to be fixed at 80 mol % using a slurry-phase system (Kreutz et al., 2008, which is incorporated herein by reference as if fully set forth). For the cobalt based units, this will represent an 80% conversion of the CO in the input stream and, for the iron rWGS units, this will represent the combined conversion of CO and CO₂ in the input stream. The fractional conversion of carbon to a given hydrocarbon product is determined using the expected amount of carbon at the product chain length (crₙ) and the information provided by the distribution of paraffin and olefin provided by Bechtel, 1998, which is incorporated herein by reference as if fully set forth.

$${cr}_{n} = \frac{n \cdot {W}_{n}}{{\sum }_{n = 1}^{29} n \cdot {W}_{n} + {n}_{Wax} \cdot {W}_{Wax}} \qquad ( 397 )$$

The iron-based FT fWGS effluent composition is based off of the slurry phase FT units developed by Mobil Research and Development Corporation in the 1980's (Mobil Research and Development Corporation, 1983; Mobil Research and Development Corporation, 1985, which are incorporated herein by reference as if fully set forth). A H₂/CO ratio of 2/3 is desired for the input feed (Mobil Research and Development Corporation, 1983; Mobil Research and Development Corporation, 1985, which are incorporated herein by reference as if fully set forth), so a sufficient amount of steam must be added to the feed to promote the forward water-gas-shift reaction. The decomposition of carbon from CO to hydrocarbons and CO2 is outlined in Table 42 of the minimal-wax FT report [(Mobil Research and Development Corporation, 1983, which is incorporated herein by reference as if fully set forth) and Table VIII-2 of the nominal-wax FT report (Mobil Research and Development Corporation, 1985, which is incorporated herein by reference as if fully set forth). This information is represented in the mathematical model using the species balance and the extent of reaction equation (Eqns. 380-381) and assuming an 90% conversion of the CO in the inlet stream (Mobil Research and Development Corporation, 1983; Mobil Research and Development Corporation, 1985, which are incorporated herein by reference as if fully set forth).

The logical use of only one type of minimal-wax FT unit is given by Equation 398 while the logical use of only one nominal-wax unit is given by Equation 399.

y_{FT-MinW—Co}+y_{FT-MinW—IrF}+y_{FT-MinW—IrR}≦1 (398)

y_{FT-NomW—Co}+y_{FT-NomW—IrF}+y_{FT-NomW—IrR}≦1 (399)

Fischer-Tropsch Upgrading

The Fischer-Tropsch vapor effluent stream must be processed to either (i) fractionate the hydrocarbon stream and upgrade each of the fractions or (ii) catalytically convert all of the hydrocarbons to gasoline-range hydrocarbons over a ZSM-5 catalyst. The wax effluent from the FT reactors will be directed to a hydrocracker to convert the wax into naphtha and distillate (Bechtel, 1998; (Mobil Research and Development Corporation, 1983; Mobil Research and Development Corporation, 1985; Baliban et al., 2011, which are incorporated herein by reference as if fully set forth).

If fractionation of the vapor effluent is desired, then the water formed during synthesis of the hydrocarbons must be initially separated from the stream. The effluent is initially sent to a water soluble oxygenates separator. It is assumed to have complete separation of the aqueous phase oxygenates (S_{APO}) (Bechtel, 1998, which is incorporated herein by reference as if fully set forth), as modeled using Equation 400. The removed oxygenates are directed to wastewater treatment while the remaining species are sent to a vapor-hydrocarbon-water separator (VLWS) unit.

N_{WSOS,VLWS,s}^{S}=0∀s∈S_{APO} (400)

The VLWS unit is modeled as a flash unit with the knockout water being sent to wastewater treatment, the vapor-phase sent to a vapor-phase oxygenates separator (VPOS) unit, and the liquid organic phase sent to a hydrocarbon recovery column (HRC) for fractionation. The VPOS unit is assumed to completely separate all remaining vapor phase oxygenates (SVPO) from the input stream (Bechtel, 1998, which is incorporated herein by reference as if fully set forth), as modeled in Equation 401. The oxygenates are sent to wastewater treatment while the remaining species exiting the VPOS unit are directed to the HRC.

N_{VPOS,HRC,s}^{S}=0∀s∈S_{VPO} (401)

The hydrocarbon fractionation and upgrading section (FIG. 44) begins by decomposing the hydrocarbons entering the HRC into C₃-C₅ gases, naphtha, kerosene, distillate, wax, offgas, and wastewater (Bechtel, 1998, Baliban et al., 2011, which are incorporated herein by reference as if fully set forth). The upgrading of each stream will follow a detailed Bechtel design (Bechtel, 1998; Bechtel, 1992, which are incorporated herein by reference as if fully set forth) which includes a wax hydrocracker, a distillate hydrotreater, a kerosene hydrotreater, a naphtha hydrotreater, a naphtha reformer, a C₄ isomerizer, a C₅/C₆ isomerizer, a C₃/C₄/C₅ alkylation unit, and a saturated gas plant.

Operating conditions of these upgrading units were not reported from Bechtel, so the mass balances for the Bechtel baseline Illinois #6 coal case study were used to determine the distribution of carbon, hydrogen, and oxygen in the effluent streams of each unit (Bechtel, 1993; Baliban et al., 2011, which are incorporated herein by reference as if fully set forth). That is, for each upgrading unit, the distribution of the input carbon is determined to either exactly match or closely approximate the distribution reported by Bechtel. The fraction of input carbon in stream (u, u′) present in each species s is given by c fu,u0,s and is reported in Table 6 of Baliban et al., 2011, which is incorporated herein by reference as if fully set forth. This is explicitly modeled for each unit in the set of all Bechtel upgrading units (U_{UG}) in Equation 402. All oxygen input to the upgrading units output as wastewater. For the wax hydrocracker, the hydrotreaters, and the isomerizers, an input of hydrogen will be required and is obtained via electrolysis or pressure-swing adsorption.

$${N}_{u , {u}^{′} , s}^{S} \cdot {AR}_{s , C} - {cf}_{u , {u}^{′} , s} \cdot {\sum }_{( {u}^{n} , u , {s}^{′} ) ∈ {S}^{UF}} {N}_{{u}^{n} , u , {s}^{′}}^{S} \cdot {AR}_{{s}^{′} , C} = 0 \\ ∀ u ∈ {U}_{UG} , ( u , {u}^{′} , s ) ∈ {s}^{UF} \qquad ( 402 )$$

The final unit in the upgrading section is the saturated gas plant. This plant operates using known recovery fractions (rfᵤ) of the C₄ species (S_{C4}) as modeled by Equation 403. The recovered C₄ species are directed back to the C₄ isomerizer while the remaining gases are sent to the light gas compressor.

$${N}_{SGP , {C}_{4} I , s}^{S} - {rf}_{s} \cdot {\sum }_{( u , SGP , s ) ∈ {S}^{UF}} {N}_{u , SGP , s}^{S} = 0 \\ ∀ s ∈ {S}_{{C}_{4}} \qquad ( 403 )$$

The FT effluent may alternatively be upgraded to gasoline-range hydrocarbons by passing the vapor over a ZSM-5 catalyst in a fixed bed reactor (Mobil Research and Development Corporation, 1983; Mobil Research and Development Corporation, 1985, which are incorporated herein by reference as if fully set forth). The composition of the effluent from the ZSM-5 unit is shown in Table 43 of the minimal-wax FT reactor Mobil study and in Table VIII-3 of the nominal-wax FT reactor Mobil study (Mobil Research and Development Corporation, 1983; Mobil Research and Development Corporation, 1985, which are incorporated herein by reference as if fully set forth). For this study, the ZSM-5 effluent composition is assumed to be equal to the composition outlined in the minimal-wax FT reactor study. This is modeled mathematically using an atom balance (Eqn. 382) around the ZSM-5 unit and the effluent composition outlined in Table 43 of the Mobil study (Mobil Research and Development Corporation, 1983, which is incorporated herein by reference as if fully set forth).

Methanol Synthesis and Conversion

The clean synthesis gas may be partially split for methanol synthesis and subsequent conversion of the methanol into liquid fuels (FIG. 45) (Mobil Research and Development Corporation, 1978; Tabak et al., 1985; Tabak et al., 1986; Tabak and Yurchak, 1990; Keil, 1999; National Renewable Energy Laboratory, 2011, which are incorporated herein by reference as if fully set forth). The methanol synthesis (MEOHS) unit will assume equilibrium between the water-gas-shift reaction (Eqn. 404) and the methanol synthesis reaction (Eqn. 405) in the effluent stream (MEOHS,u) (National Renewable Energy Laboratory, 2011, which is incorporated herein by reference as if fully set forth).

N_{MEOHS,u,H}_{₂}_{O}^{S}·N_{MEOHS,u,CO}^{S}=K_{MEOHS}^{WGS}·N_{MEOHS,u,H}_{₂}^{S}·N_{MEOHS,u,CO}_{₂}^{S} (404)

N_{MEOHS,u,CH}_{₃}_{OH}^{S}=K_{MEOHS}^{MSN}·N_{MEOHS,u,H}_{₂}^{S}^{²}·N_{MEOHS,u,CO}^{S} (405)

The raw methanol effluent is degassed (MEDEG) to remove any light vapors. The MEDEG unit is operated as a split unit and assumes that the entrained vapor will be completely removed from the methanol (Eqn. 406) and that the methanol will completely remain as a liquid (Eqn. 407).

N_{MEDEG,SP}_{_{MEOH}}_{,s}^{S}=0∀s≠CH₃OH (406)

N_{MEDEG,SP}_{_{MEOH}}_{,CH}_{₃}_{OH}^{S}=N_{MEOHS,MEDEG,CH}_{₃}_{OH}^{S} (406)

The purified methanol is split to either the methanol-to-gasoline (MTG) process or to the methanol-to-olefins (MTO) and Mobil olefins-to-gasoline/distillate (MOGD) processes, both of which were developed by Mobil Research and Development in the 1970's and 1980's. More recently, the National Renewable Energy Laboratory performed a full design, simulation, and economic analysis of a biomass-based MTG process (National Renewable Energy Laboratory, 2011, which is incorporated herein by reference as if fully set forth). The MTG process will catalytically convert the methanol to gasoline range hydrocarbons using a ZSM-5 zeolite and a fluidized bed reactor. The MTG effluent is outlined in Table 3.4.2 of the Mobil study (Mobil Research and Development Corporation, 1978, which is incorporated herein by reference as if fully set forth) and in Process Flow Diagram P850-A1402 of the NREL study (National Renewable Energy Laboratory, 2011, which is incorporated herein by reference as if fully set forth). Due to the high level of component detail provided by NREL for both the MTG unit and the subsequent gasoline product separation units, the composition of the MTG reactor used in this study is based on the NREL report. The MTG unit will operate adiabatically at a temperature of 400° C. and 12.8 bar. The methanol feed will be heated to 330° C. and input to the reactor at 14.5 bar. The MTG effluent will contain 44 wt % water and 56 wt % crude hydrocarbons, of which 2 wt % will be light gas, 19 wt % will be C₃-C₄ gases, and 19 wt % will be C₅₊ gasoline (National Renewable Energy Laboratory, 2011, which is incorporated herein by reference as if fully set forth). The crude hydrocarbons will ultimately be separated into finished fuel products, of which 82 wt % will be gasoline, 10 wt % will be LPG, and the balance will be recycle gases. This is modeled mathematically in the process synthesis model by using an atom balance around the MTG unit and assuming a 100% conversion of the methanol entering the MTG reactor (Mobil Research and Development Corporation, 1978; National Renewable Energy Laboratory, 2011, which are incorporated herein by reference as if fully set forth).

Any methanol entering the MTO process unit is heated to 400° C. at 1.2 bar. The MTO fluidized bed reactor operates at a temperature of 482° C. and a pressure of 1 bar. The exothermic heat of reaction within the MTO unit is controlled through generation of low-pressure steam. 100% of the input methanol is converted into olefin effluent containing 1.4 wt % CH₄, 6.5 wt % C₂-C₄ paraffins, 56.4 wt % C₂-C₄ olefins, and 35.7 wt % C₅-C₁₁ gasoline (Tabak and Yurchak, 1990, which is incorporated herein by reference as if fully set forth). The MTO unit is modeled mathematically using an atom balance and a typical composition seen in the literature (Tabak and Yurchak, 1990, which is incorporated herein by reference as if fully set forth). The MTO product is fractionated (MTO-F) to separate the light gases, olefins, and gasoline fractions. The MTO-F unit is assumed to operate as a separator unit where 100% of the C₁-C₃ paraffins are recycled back to the refinery, 100% of the C₄ paraffins and 100% of the olefins are directed to the MOGD unit, 100% of the gasoline is combined with the remainder of the gasoline generated in the process, and 100% of the water generated in the MTO unit is sent for wastewater treatment.

The separated olefins are sent to the MOGD unit where a fixed bed reactor is used to convert the olefins to gasoline and distillate over a ZSM-5 catalyst. The gasoline/distillate product ratios can range from 0.12 to >100, and the ratio chosen in this study was 0.12 to maximize the production of diesel. The MOGD unit operates at 400° C. and 1 bar and will utilize steam generation to remove the exothermic heat of reaction within the unit. The MOGD unit is modeled with an atom balance and will produce 82% distillate, 15% gasoline, and 3% light gases (Tabak and Yurchak, 1990, which is incorporated herein by reference as if fully set forth). The product will be fractionated (MTODF) to remove diesel and kerosene cuts from the gasoline and light gases. The MTODF unit will be modeled as a separator unit where 100% of the C₁₁-C₁₃ species are directed to the kerosene cut and 100% of the C₁₄₊ species are directed to the diesel cut.

LPG-Gasoline Separation

The LPG and gasoline generated from ZSM-5 conversion of the FT hydrocarbons or the methanol must be passed through a series of separation units to extract the LPG from the gasoline and alkylate any iso-butane to a blending stock for the final gasoline pool (FIG. 46). Light gases are initially removed via one of two knock-out units, and the crude hydrocarbons are passed through a deethanizer column, a stabilizer column, an absorber column, a splitter column, and an LPG alkylate splitter to separate the LPG from the gasoline fractions. Each of these units is modeled mathematically as a splitter unit where the split fraction of each species to an output stream is given by the information in the Process Flow Diagrams P850-A1501 and P850-A1502 from the NREL study (National Renewable Energy Laboratory, 2011, which is incorporated herein by reference as if fully set forth). All low pressure steam and cooling water needed for each of the units is derived for each of the units in the NREL study. The total amount of process utility that is needed per unit flow rate from the top or bottom of the column is calculated, and this ratio is used as a parameter in the process synthesis model to determine the actual amount of each utility needed based on the unit flow rate.

In addition to the distillation columns within this section, there is also an alkylation unit that is used to convert iso-butane and butene to an alkylate blending stock for the gasoline pool. The alkylate was modeled as iso-butane (National Renewable Energy Laboratory, 2011, which is incorporated herein by reference as if fully set forth) and the alkylation unit was modeled using a species balance where the key species, butene, was completely converted to iso-butane. Butene is used as the limiting species in this reaction because it is generally present in a far smaller concentration than iso-butane.

### Example 3.23

### Feedstock Analyses

The proximate and ultimate analyses for each of the coal and biomass feedstocks are included in Table 46. The composition of the low-volatile bituminous coal is obtained from the NETL Quality Guidelines Report (National Energy Technology Laboratory, 2004, which is incorporated herein by reference as if fully set forth) and the composition of the switchgrass is obtained from the ECN Phyllis database (van der Drift and van Doorn, 2002, which is incorporated herein by reference as if fully set forth). The molar composition of the natural gas feedstock is included in Table 47 and is derived from the Quality Guidelines Report (National Energy Technology Laboratory, 2004, which is incorporated herein by reference as if fully set forth).

TABLE 46
Feedstock proximate and ultimate analysis for biomass and coal.

| | Proximate Analysis (db, Weight %) | | | | Heating values (kJ/kg) | |
|---|---|---|---|---|---|---|
| Feed Type | Moist (ar) | Ash | VM¹ | FC² | HHV³ | LHV⁴ |
| Low-volatile Bituminous | 0.65 | 4.77 | 19.26 | 75.97 | 34946 | 34012 |
| Switchgrass | 8.2 | 4.6 | 79.2 | 16.2 | 18636 | 17360 |
| | Ultimate Analysis (db, weight %) | | | | | |
| Feed Type | C | H | N | Cl | S | O |
| Low-volatile Bituminous | 86.71 | 4.23 | 1.27 | 0.19 | 0.66 | 2.17 |
| Switchgrass | 46.9 | 5.85 | 0.58 | 0.501 | 0.11 | 41.5 |

¹VM = volatile matters;
²FC = fixed carbon;
³HHV = higher heating value;
⁴LHV = lower heating value

TABLE 47
Molar compositions (x) of all species in the input natural gas.

| Species | x | Species | x | Species | x |
|---|---|---|---|---|---|
| CH₄ | 0.931 | C₂H₆ | 0.032 | N₂ | 0.016 |
| CO₂ | 0.010 | C₃H₈ | 0.007 | nC₄H₁₀ | 0.004 |

### Example 4

### Novel Natural Gas to Liquids Processes: Process Synthesis and Global Optimization Strategies

An optimization-based process synthesis framework is proposed for the conversion of natural gas to liquid transportation fuels. Natural gas conversion technologies including steam reforming, autothermal reforming, partial oxidation to methanol, and oxidative coupling to olefins are compared to determine the most economic processing pathway. Hydrocarbons are produced from Fischer-Tropsch (FT) conversion of syngas, ZSM-5 catalytic conversion of methanol, or direct natural gas conversion. Multiple FT units with different temperatures, catalyst types, and hydrocarbon effluent compositions are investigated. Gasoline, diesel, and kerosene are generated through upgrading units involving carbonnumber fractionation or ZSM-5 catalytic conversion. A powerful deterministic global optimization method is introduced to solve the mixed-integer nonlinear optimization model that includes simultaneous heat, power, and water integration. Twenty-four case studies are analyzed to determine the effect of refinery capacity, liquid fuel composition, and natural gas conversion technology on the overall system cost, the process material/energy balances, and the life cycle greenhouse gas emissions.

This example discloses an optimization-based process synthesis framework for directly comparing the technoeconomic and environmental benefits of GTL processes in a singular mathematical model. The framework is capable of simultaneously analyzing several existing or novel, processes via a process superstructure to determine the optimal topology that will have either the lowest cost or highest net present value. A rigorous global optimization strategy is used to mathematically guarantee that the process design selected by the framework will have an overall cost (or profit) that is within a small percentage of the best value possible. The disclosure in this example includes (1.) the inclusion and mathematical modeling of steam reforming of natural gas, direct conversion of natural gas to methanol via partial oxidation, and direct conversion of natural gas to olefins via oxidative coupling (OC) as conversion technologies, in addition to autothermal reforming (ATR), (2) the direct usage of natural gas in the fuel combustor unit to provide process heat and in the gas turbine (GT) for electricity production, (3) different product compositions gasoline, diesel, and kerosene) considered, namely the unrestricted composition, maximization of diesel, maximization of kerosene, and compositions commensurate with the U.S. demand ratio, and (4) calculations of the life cycle emissions of GTL systems compared with petroleum-based processes and natural gas-based electricity production. The framework includes a simultaneous heat and power integration using an optimization-based heat-integration approach and a series of heat engines that can convert waste heat into electricity. A comprehensive wastewater treatment network that utilizes a superstructure approach to determine the appropriate topology and operating conditions of process units is utilized to minimize wastewater contaminants and freshwater intake.

The process synthesis framework will be utilized to examine (1) natural gas conversion via steam reforming, ATR, direct conversion to methanol, and direct conversion to olefins, (2) synthesis gas conversion via Fischer-Tropsch (FT) or methanol synthesis, (3) methanol conversion via methanol-togasoline (MTG) or methanol-to-olefins (MTO), and (4) hydrocarbon upgrading via ZSM-5 zeolite catalysis, olefin oligomerization, or boiling point fractionation and subsequent treatment. The key products from the GTL refinery will be gasoline, diesel, and jet fuel (kerosene) with allowable byproducts of liquefied petroleum gas (LPG-) and electricity.

### Example 4.1

### GTL Process Superstructure: Conceptual Design and Mathematical Modeling

This example will detail the modeling required to introduce additional means for natural gas conversion and the subsequent processing of the effluent streams. The complete mathematical model including all relevant nomenclature is provided as Example 3.16, whereas the full set of process flow diagrams (PFDs) are provided as Example 4.15.

Natural Gas Conditioning

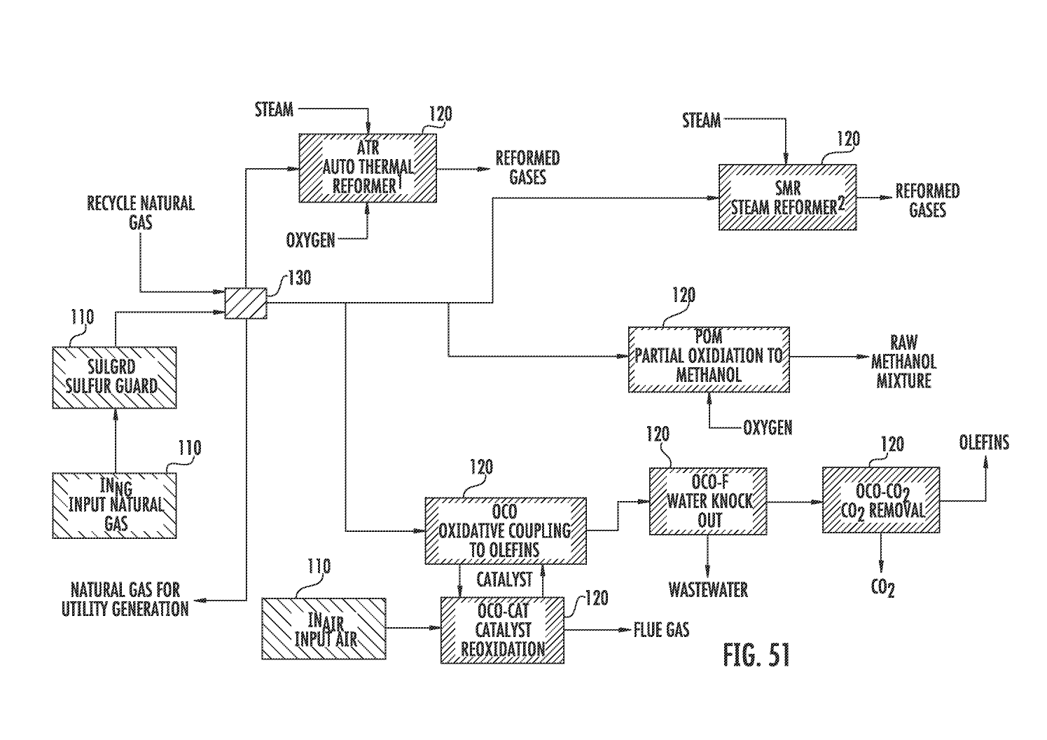

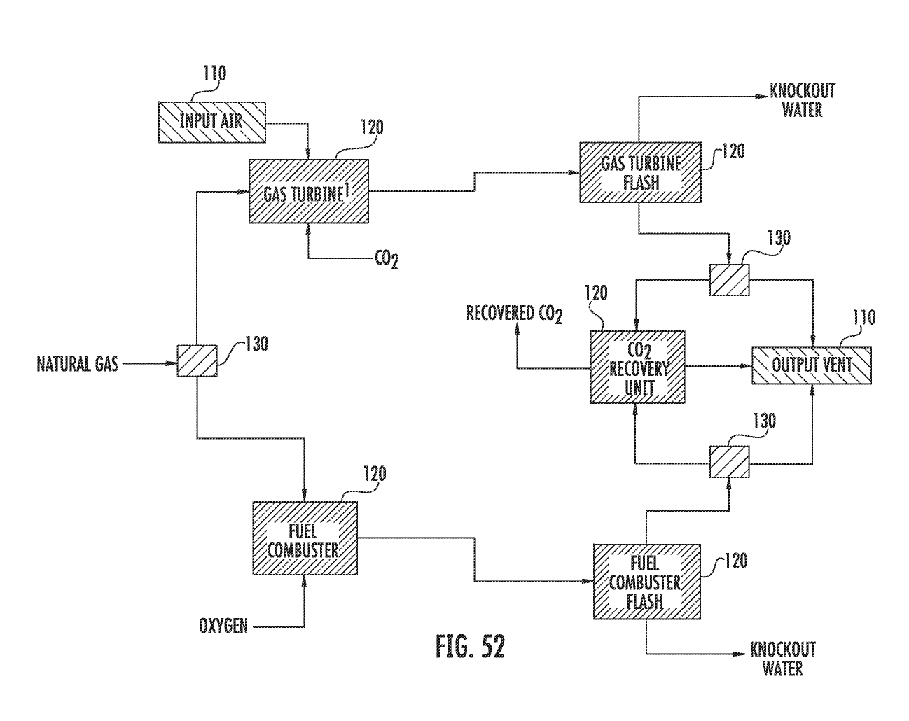

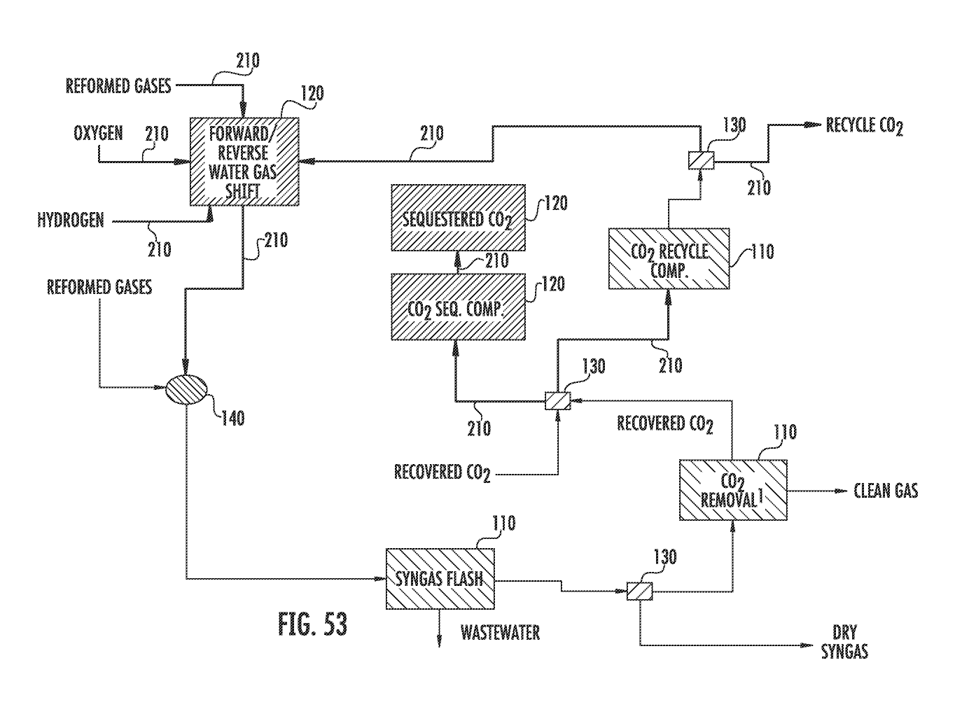

Natural gas is fed to the GTL refinery at pipeline conditions of 31 bar and 25° C. and is utilized in one of six major processes including ATR, steam reforming, direct coversion to methanol, direct conversion to olefins, fuel combustion, and GT electricity generation. (FIGS. 51 and 52). The input natural gas composition (Table 48) is taken from the NETL Quality Guidelines for Energy Systems Studies Report and is based on the mean of over 6800 samples of pipeline quality natural gas (National Energy Technology Laboratory, 2004; National Energy Technology Laboratory, 2010, which are incorporated herein by reference as if fully set forth). Natural gas must be desulfurized to protect the catalysts in the GTL refinery, though the low sulfur concentration in pipeline natural gas (˜6 ppmv57) will negate the need for desulfurization technology. A zinc oxide polishing bed (sulfur guard) is used to clean any mercaptan-based odorizers from the gas to prevent catalyst contamination.56 Naturalgas and other methane-rich recycle gases may be sent to a GT to produce electricity or to a fuel combustor to provide process heat (FIG. 52). CO₂ produced from these units may be captured and mixed with additional process CO₂ for appropriate handling (FIG. 53).

TABLE 48
Molar Compositions (x) of all species in the input natural gas

| Species | x |
|---|---|
| CH₄ | 0.931 |
| CO₂ | 0.010 |
| C₂H₆ | 0.032 |
| C₃H₈ | 0.007 |
| N₂ | 0.016 |
| n-C₄H₁₀ | 0.004 |

Natural Gas Conversion

The natural gas leaving the sulfur guard may be converted to synthesis gas (syngas; CO, CO₂, H₂, and H₂O) via steam reforming (steammethane-reforming [SMR]) or ATR. Both these reforming reactors will assume an equilibrium is reached for SMR (Eq. (408)) and the water-gas-shift (WGS) reaction (Eq. (409)). The effluent concentrations of C₂ and higher hydrocarbons are assumed to be negligible with respect to the concentration of methane.

CH₄+H₂O [symbol] CO+3H₂ (408)

CO₂+H₂ [symbol] CO+H₂O (409)

Steam Reforming.

Steam reforming of the natural gas uses a nickel-based catalyst contained inside high alloy steel tubes. Beat is provided for the endothermic reforming of methane via combustion of recycle fuel gas and additional input natural gas over the outside of the tubes. The reformer operates at a pressure of 30 bar with typical reaction temperatures of 700-900° C. The effluent reformed gas will be constrained by both WGS equilibrium (Eq. (410)) and SMR equilibrium (Eq. (411)). The WGS equilibrium conserves the total molar flow rate, so the species molar flow rates (N^{S}) are sufficient to accurately define the equilibrium constraint. The SMR equilibrium constraint utilizes molar species concentrations (x^{s}) to account for the change in total molar flow rate. The equilibrium constant in Eq. (411) was adjusted from the value extracted from Aspen Plus for the higher pressure of the reforming unit. Although additional methods exist for defining the constraints in the steam reformer (e.g., molar species concentrations in WGS equilibrium), the current mathematical formulation for the steam reformer provided the best computational performance for this study. All nonhydrocarbon and nonsyngas species (e.g., N₂ and Ar) are assumed to be inert. The effluent reformed gas is directed to syngas cleaning (see FIG. 53). Ambient air (13° C., 1.01 bar) is compressed to 1.1 bar to provide a 20 mol % stoichiometric excess of oxygen needed for combustion of the fuel gas within the reformer. The combusted fuel gas exits the reformer at 640° C., is cooled to 120° C. to recover waste heat, and is then directed to either the stack or a CO₂ recovery unit.

N_{SMR,u,H}_{₂}_{O}^{S}·N_{SMR,u,CO}^{S}=K_{SMR}^{WGS}·N_{SMR,u,H}_{₂}^{S}·N_{SMR,u,CO}_{₂}^{S} (410)

x_{SMR,u,CH}_{₄}^{S}·x_{SMR,u,H}_{₂}_{O}^{S}=K_{SMR}^{MR}·x_{SMR,u,H}_{₂}^{S}^{³}·x_{SMR,u,CO}^{S} (411)

Auto-Thermal Reforming.

ATR of the natural gas will input a combination of steam for endothermic reforming and high-purity oxygen for partial combustion within the same reactor. The autothermal reformer will, operate at a pressure of 30 bar with a temperature between 700 and 1000° C. Oxygen is provided through cryogenic air separation (99.5 wt %) or electrolysis of water (100 wt %) and is preheated to 300° C. prior to entering the reformer. Steam will also be preheated to 550° C., and the natural gas will be preheated to 300° C. to reduce the oxygen requirement within the reformer. The molar ratio of steam to total carbon entering the reformer will vary between 0.5 and 1.5, and the effluent will be governed by the WGS equilibrium (Eq. (412)) and SMR equilibrium (Eq. (413)). The choice of mathematical formulation of the autothermal effluent is similar to that of the steam reformer and is based on computational performance.

N_{ATR,u,H}_{₂}_{O}^{S}·N_{ATR,u,CO}^{S}=K_{ATR}^{WGS}·N_{ATR,u,H}_{₂}^{S}·N_{ATR,u,CO}_{₂}^{S} (412)

x_{ATR,u,CH}_{₄}^{S}·x_{ATR,u,H}_{₂}_{O}^{S}=K_{ATR}^{MR}·x_{ATR,u,H}_{₂}^{S}^{³}·x_{ATR,u,CO}^{S} (413)

The effluent from the autothermal reformer is directed to the synthesis gas cleaning section (FIG. 53).

Direct Conversion to Methanol Via Partial Oxidation.

Natural gas may be directly converted to methanol via gas-phase partial oxidation operated by a free radical mechanism. The natural gas is compressed to 52 bar and then passed into a quartz-lined tubular reactor (POM) operating at 4.50° C. and 50 bar. The per-pass conversion of methane (fc) is 13%59 (Eq. (414)) with a carbon distribution (cd) of 63% to CH₃OH, 30% to CO, 6% to CO₂, and 1% to C₂H₆57 (Eq. (415)), where S_{POM}^{Ef} represents the set of species that are formed from conversion of the methane. Under the reaction conditions assumed in this study, all formaldehyde is assumed to decompose quickly to H₂ and CO.57 Oxygen is provided via an air separation unit (99.5 wt %) or electrolysis (100 wt %) with subsequent compression to 52 bar.

$$\begin{matrix}{N}_{POM , u , {CH}_{4}}^{S} = {fc}_{POM , {CH}_{4}} \cdot {\sum }_{( {u}^{′} , POM , {CH}_{4} ) ∈ {S}^{UF}} {N}_{{u}^{′} , POM , {CH}_{4}}^{S} & ( 414 ) \\ {N}_{POM , u , s}^{S} \cdot {AR}_{s , C} = {cd}_{POM , {CH}_{4}} \cdot {\sum }_{( {u}^{′} , POM , {CH}_{4} ) ∈ {S}^{UF}} {N}_{{u}^{′} , POM , {CH}_{4}}^{S} \\ ∀ s ∈ {S}_{POM}^{Ef} & ( 415 )\end{matrix}$$

The effluent from the reactor is combined with the effluent from the methanol generated from synthesis gas, cooled to 35° C., and flashed to separate the methanol/water mixture. The recycle gases are either (1) recompressed and recycled to the POM reactor, (2) heated to 500° C. and expanded to 30 bar for use in a GT, or (3) heated to 500° C. and expanded to 1.3 bar for use as fuel gas. The crude methanol/water mixture is combined with additional methanol from the plant prior to degassing and subsequent processing.

Direct Conversion to Olefins Via OC.

Natural gas can be contacted with a reducible metal oxide catalyst to promote oxidative dehydrogenation via free radical formation. The reactor (OCO) is assumed to operate at 800° C. and 3.8 bar64 with suitable expansion of the natural gas to recover electricity from a turbine. A typical CH₄ conversion (fc) over a 15% Mn, 5% Na₄P₂O_{y}/SiO₂ catalyst is 22% with a 77% selectivity (cd) to C₂₊ hydrocarbons.

$$\begin{matrix}{N}_{OCO , u , s}^{S} = {fc}_{OCO , s} \cdot {\sum }_{( {u}^{′} , OCO , s ) ∈ {S}^{UF}} {N}_{{u}^{′} , OCO , s}^{S} \\ ∀ s ∈ {S}_{OCO}^{HC} & ( 416 ) \\ {N}_{OCO , u , s}^{S} \cdot {AR}_{s , C} = {cd}_{OCO , s} \cdot {\sum }_{s ∈ {S}_{OCO}^{HC}} {\sum }_{( {u}^{′} , OCO , s ) ∈ {S}^{UF}} {N}_{{u}^{′} , OCO , s}^{S} & ( 417 ) \\ ∀ s ∈ {S}_{OCO}^{Ef} & ( 10 )\end{matrix}$$

This assumes that the per-pass conversion of CH₄ is 25% (Eq. (416)) with a product composition shown in Table 49. The distribution of paraffins and olefins for C₂-C₅ hydrocarbons was assumed to be equal to that of the C₂ species, and the C₄-C₅ species were assumed to be linear. Equation (417) shows the mathematical constraint for distribution of carbon from the input hydrocarbons (S_{OCO}^{HC}) to all effluent species (S_{OCO}^{Ef}) in the reactor. The per-pass conversion of other light paraffins (e.g., CₙH₂ₙ₊₂) is also assumed to be 25% with a carbon distribution to CO, CO₂, coke, and Cₙ₊ equivalent to that in Table 49, below.

TABLE 49
Product selectivity for OC of natural gas using a 15% Mn, 5%
Na₂P₂O_{y}/SiO₂ catalyst

| | |
|---|---|
| Temperature (° C.) | 800 |
| % CH₄ conversion | 15 |
| % Selectivity of carbon | |
| C₂H₄ | 47.0 |
| C₂H₆ | 14.0 |
| C₃H₆ | 4.6 |
| C₃H₈ | 1.4 |
| n-C₄H₈ | 3.1 |
| n-C₄H₁₀ | 0.9 |
| n-C₅H₁₀ | 0.8 |
| n-C₅H₁₂ | 0.2 |
| Benzene | 4 |
| Toluene | 0.4 |
| CO | 11 |
| CO₂ | 11 |
| Coke | 1 |

The catalyst is regenerated (OCO-CAT) by passing air (10% stoichiometric excess of O₂) over the catalyst surface for reoxidation and removal of the coke to CO₂. The flue gas is cooled to 120° C. to recover waste heat and is either vented or sent to a CO₂ recovery unit. The effluent of the reactor is cooled to 35° C. for water knock-out (OCO—F), compressed to 50 bar, and then sent to a CO₂ removal unit (OCO—CO₂). The effluent from the CO₂ removal unit is then directed to the Mobil olefins-to-gasoline/distillate (MOOD) reactor to generate gasoline and distillate. A summary of the operating conditions within each of the four natural gas conversion units is shown in. Table 50, below.

TABLE 50
Operating conditions for the direct or indirect conversion
of natural gas

| | Temperature | Pressure | Conv. of |
|---|---|---|---|
| Unit | (° C.) | (bar) | CH₄(%) |
| Autothermal | 700-1000 | 30 | 80-95 |
| reformer | | | |
| Steam reformer | 700-900 | 30 | 80-95 |
| Partial oxidation | 450 | 50 | 13 |
| OC | 800 | 3.8 | 25 |

### Example 4.2

### Synthesis Gas Cleaning

The PFD for processing the raw syngas from the SMR or ATR reactors is shown in FIG. 53. The syngas effluent from the steam reformer or the autothermal reformer may require a forward or reverse WGS reaction depending on the reformer effluent composition and the input feed requirements for the FT or methanol synthesis. Additionally, the use of reverse WGS may provide a means for CO₂ conversion using H₂ that is either present in the input stream or recycled from the process. The WGS unit will operate at a pressure of 28 bar and a temperature between 400 and 600° C. The effluent from the WGS reactors is cooled to 35° C. and sent to a water knock-out unit operating at 27.5 bar where vapor-liquid equilibrium is used to separate most of the water from the synthesis gas. The vapor effluent from the flash unit may be split to (1) a CO₂ recovery unit (e.g., one-stage Rectisol) to remove 90% of the CO₂ in the syngas or (ii) directly passed the hydrogen production/upgrading section. The clean syngas from the CO₂ recovery unit exits at 35° C. and 27 bar and is sent to the hydrocarbon production/upgrading section. The CO₂ from the Rectisol unit exits at 1.5 bar and 49° C. and may be (a) compressed to 31 bar for recycle to the reformers or the WGS units or (b) compressed to 1.50 bar for sequestration. Note that both compression options will utilize multiple compression stages with intercooling to control the temperature rise. The CO₂ may alternatively be vented to the atmosphere.

### Example 4.3

### Hydrocarbon Production/Upgrading

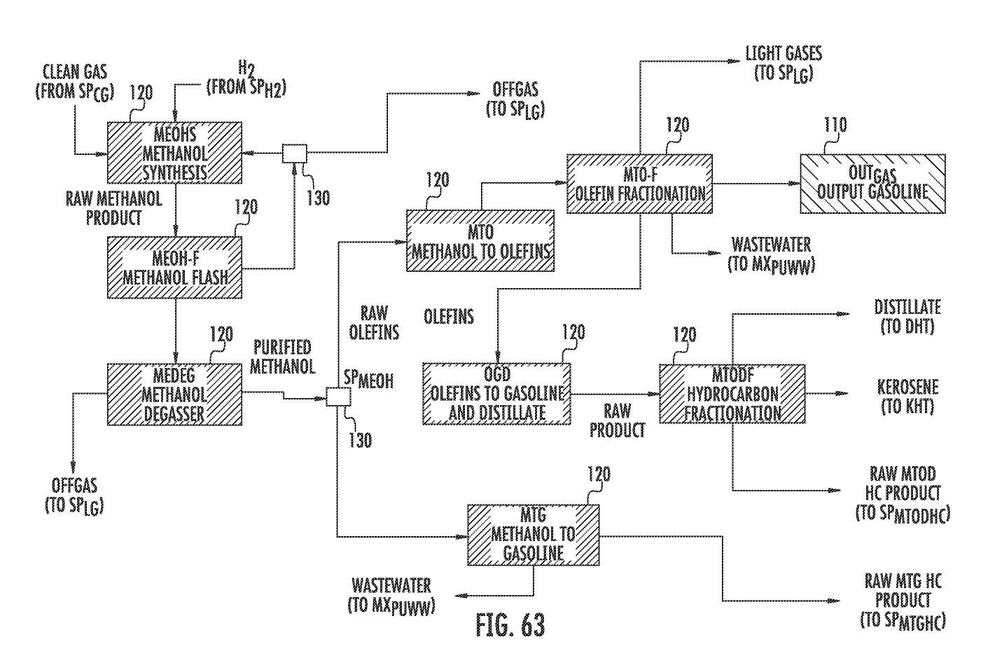

FT Hydrocarbon Production. The hydrocarbon production section (FIGS. 60 and 63) will convert the syngas using either FT synthesis or methanol synthesis. The FT units will operate at 20 bar and will utilize either a cobalt-based or iron-based catalyst.14,15,30 The cobalt-based units will require a CO₂-lean synthesis gas feed to prevent poisoning of the FT catalyst and increase conversion of the CO. The iron-based catalysts may use either the CO₂-lean or CO₂-rich syngas, because the WGS reaction will be facilitated by the iron catalyst. Therefore, these reactors could consume CO₂ within the unit using H₂ to produce the CO necessary for the FT reaction.

Synthesis gas is split to either the low-wax FT section (SP_{FTM}), the nominal wax FT section (SPFTN), or methanol synthesis (MEOHS). The FT units will operate within the temperature range of 240-320° C. The cobalt-based FT units operate at either low temperature (LTFT; 240° C.) or high temperature (HTFT; 320° C.) and must have a minimal amount of CO₂ in the input stream. Two iron-based FT units will facilitate the WGS reaction and will operate at low (LTFTRGS; 240° C.) and high temperature (HTFTRGS; 320° C.). The other two iron-based FT units will operate at a mid-level temperature (267° C.), and produce either minimal (MTFTWGS-M) or nominal (MTFTWGS-N) amounts of wax. Each of the four iron-based FT units may facilitate either the forward or the reverse WGS reaction.

Hydrogen may be recycled to any of the FT units to either shift the H₂/CO ratio or the H₂/CO₂ ratio to the appropriate level. Steam may alternatively be used as a feed for the two iron-based fWGS FT units to shift the H2/CO ratio, CO₂ may be recycled back to the iron-based FT units to be consumed in the WGS reaction. Similarly, the pressure-swing adsorption (PSA) offgas, which will be lean in H₂, may be recycled to the iron-based FT units for consumption of the CO or CO₂. The effluent from the autothermal reactor (ATE) will contain a H₂/CO ratio that is generally above 2/1 and is, therefore, favorable as a feedstock for FT synthesis. However, the concentration of CO₂ within the ATE effluent will prevent the stream from being fed to the cobalt-based units. The two streams exiting the FT units will be a waxy liquid phase and a vapor phase containing a range of hydrocarbons. The wax will be directed to a hydrocracker (WHC), whereas the vapor phase is split (SP_{FTH}) for further processing.

FT Hydrocarbon Upgrading.

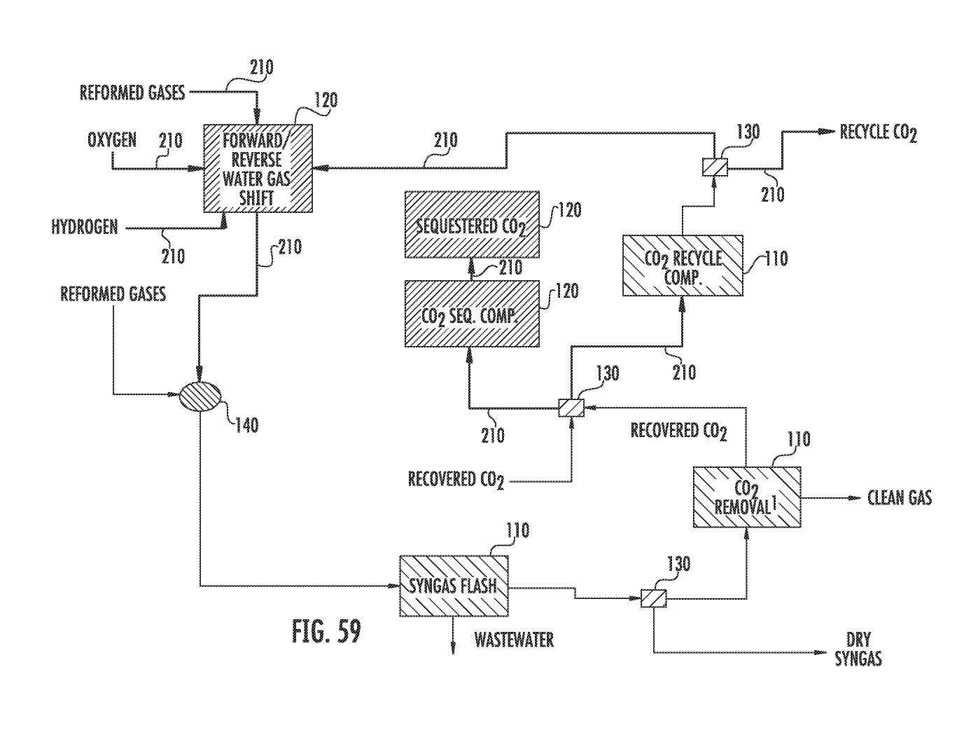

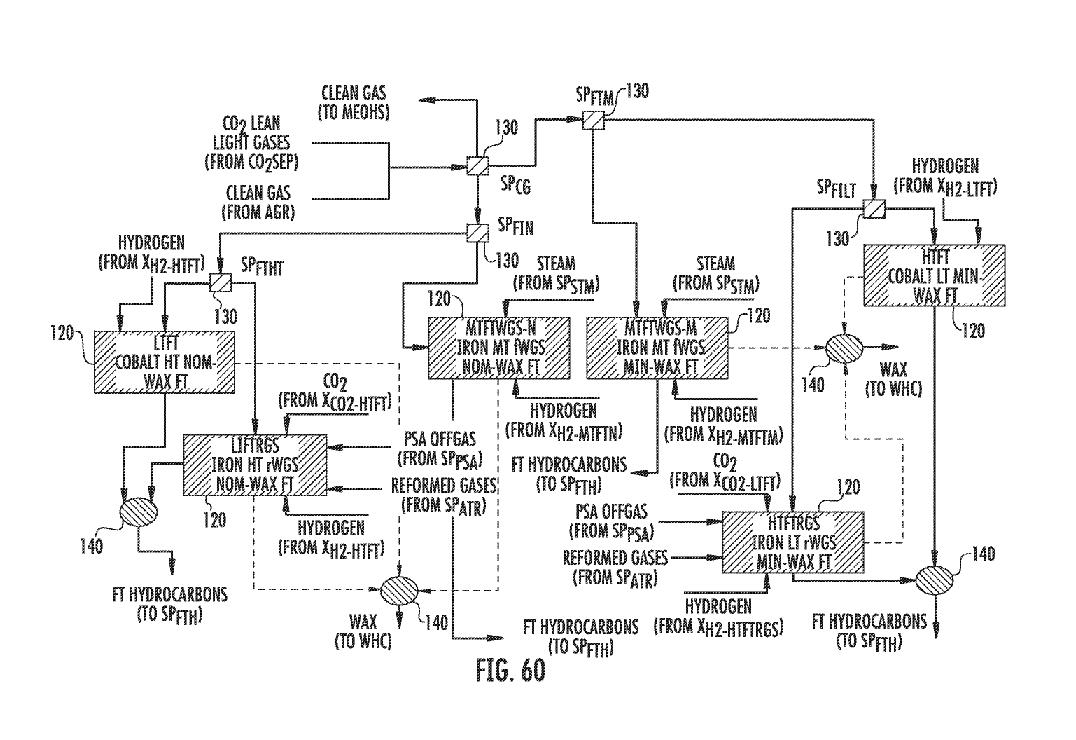

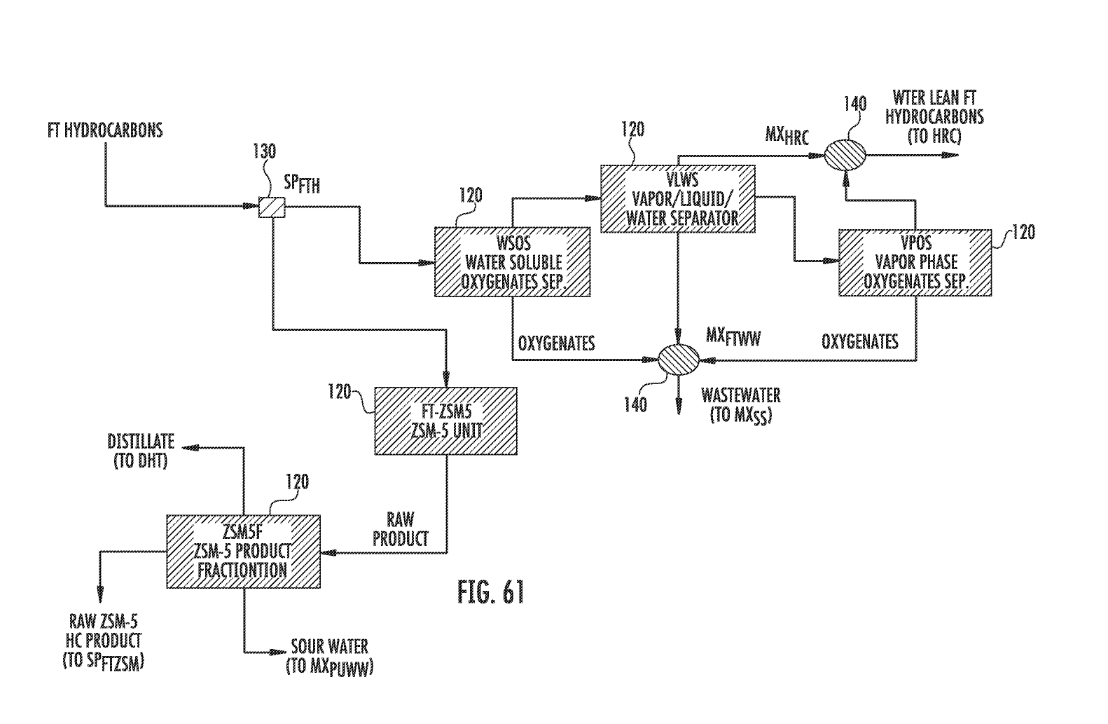

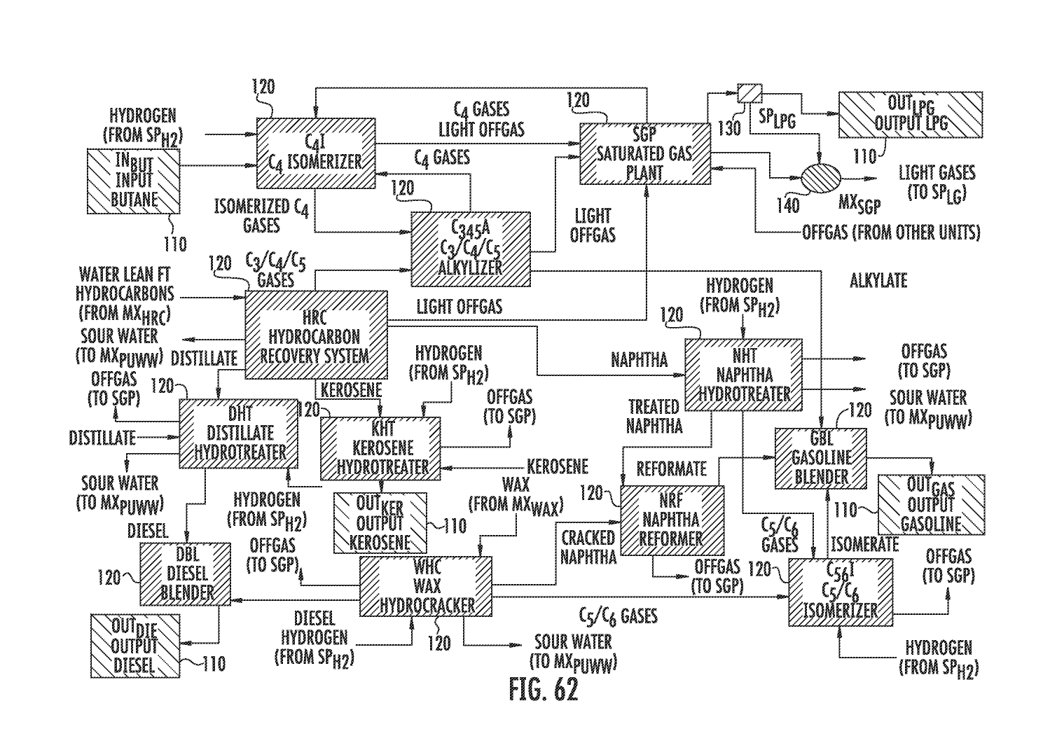

The vapor phase effluent from FT synthesis will contain a mixture of C₁-C₃₀₊ hydrocarbons, water, and some oxygenated species. FIG. 61 details the process flow sheet used to process this effluent stream. The stream will be split and can pass through a series of treatment units designed to cool the stream and knock out the water and oxygenates for treatment. Initially, the water-soluble oxygenates are stripped from the stream. The stream is then passed to a three-phase separator to remove the aqueous phase from the residual vapor and any hydrocarbon liquid. Any oxygenates that are present in the vapor phase may be removed using an additional separation unit. The water lean FT hydrocarbons are then sent to a hydrocarbon recovery column for fractionation and further processing (FIG. 62). The oxygenates and water removed from the stream are mixed and sent to the biological digester for wastewater treatment.

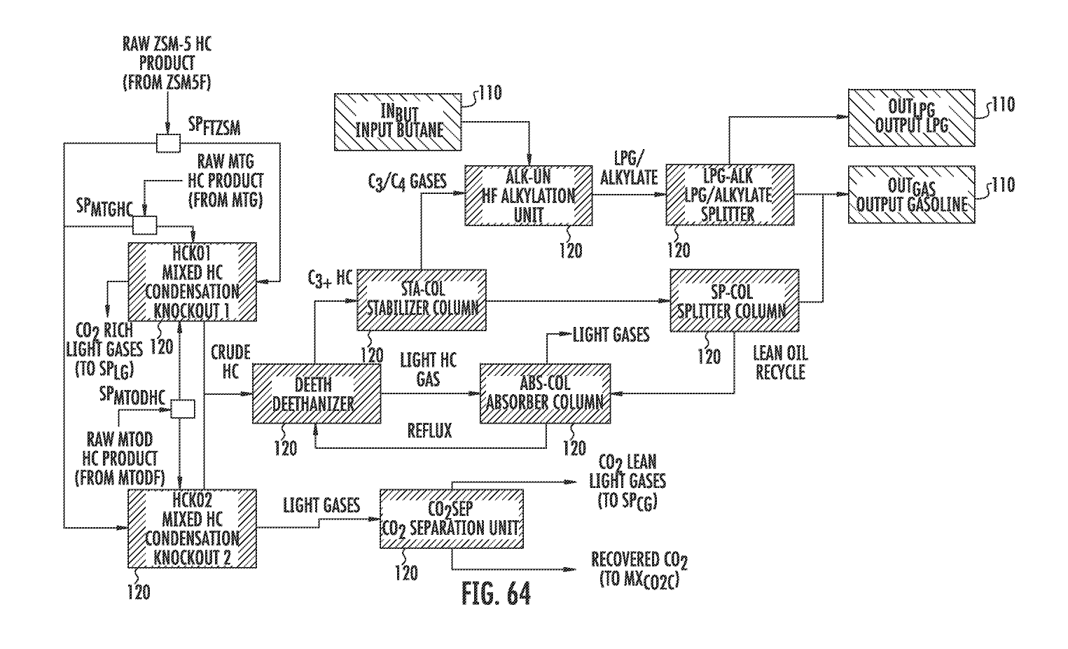

The FT hydrocarbons may also be passed over a ZSM-5 catalytic reactor operating at 408° C. and 16 bar to be converted into mostly gasoline range hydrocarbons and some distillate. The ZSM-5 unit will be able to convert the oxygenates to additional hydrocarbons, so no separate processing of the oxygenates will be required for the aqueous effluent. The raw product from FT-ZSM5 is fractionated to separate the water and distillate from the gasoline product. The water is mixed with other wastewater knock-out, and the distillate is hydrotreated to form a diesel product. The raw ZSM-5 HC product is sent to the LPG-gasoline separation section for further processing (FIG. 64).

The water lean FT hydrocarbons are sent to a hydrocarbon recovery column, as shown in FIG. 62. The hydrocarbons are split into C₃-C₅ gases, naphtha, kerosene, distillate, wax, offgas, and wastewater.12,70 The upgrading of each stream will follow a detailed Bechtel design (Bechtel 1998; Bechtel 1992, which are incorporated herein by reference as if fully set forth), which includes a wax hydrocracker, a distillate hydrotreater, a kerosene hydrotreater, a naphtha hydrotreater, a naphtha reformer, a C₄ isomerizer, a C₅/C₆ isomerizer, a C₃/C₄/C₅ alkylation unit, and a saturated gas plant.

Methanol Synthesis.

The methanol synthesis reactor (FIG. 63) will operate at 300° C. and 50 bar and may input either the CO₂-rich or CO₂-lean syngas. The syngas leaving the cleaning section must be compressed to 51 bar prior to entering the methanol synthesis reactor. The methanol synthesis reactor will assume equilibrium is achieved for the WGS reaction (Eq. (419)) and the methanol synthesis reaction (Eq. (418)).

CO+2H₂ [symbol] CH₃OH (418)

CO₂+H₂ [symbol] CO+H₂O (419)

The typical per-pass conversion of CO and CO₂ methanol is ˜35%, and the relative concentration of H₂O to methanol in the effluent stream is largely determined based on the input concentration of CO₂ to the reactor. The effluent from the reactor is cooled to 35° C., and a crude methanol stream is separated using vapor-liquid equilibrium at 48 bar. The amount of methanol that is entrained in the vapor phase is dependent on the input concentration of syngas to the flash unit, but a majority (over 95%) of the methanol can be recovered by enforcing a stoichiometric amount of H₂ in the input to the synthesis reactor H₂/(2CO+3CO₂)=1). The vapor stream from the flash unit is split, so that 5% may be purged to remove inert species, and the remaining 95% is compressed to 51 bar and then recycled to the methanol synthesis reactor. The purge stream is recycled back to the process and used as fuel gas.

The crude methanol product from the flash unit is heated to 200° C., expanded to 5 bar to recover electricity, and then cooled to 60° C. prior to entering a degasser distillation column. The degasser will remove all the entrained gases from the liquid methanol/water while recovering 99.9% of the methanol. The entrained gases are recycled back to the process for use as fuel gas. The bottoms from the degasser will contain methanol and water, with a methanol composition dependent on the level of CO₂ input to the synthesis unit. High levels of water in the liquid stream are not anticipated to be a concern, because the downstream methanol processing units will yield 50 wt % water from the hydrocarbon synthesis.

Methanol Conversion.

The purified methanol is split to either the MTG process or to the MTO and MOGD processes. The MTG process will catalytically convert the MTG range hydrocarbons using a ZSM-5 zeolite and a fluidized bed reactor. The MTG effluent is outlined in Table 3.4.2 of the Mobil study (Mobil Research and Development Corporation, 1978, which is incorporated herein by reference as if fully set forth) and in PFD P850-A1402 of the NREL study (National Renewable Energy Laboratory, 2011, which is incorporated herein by reference as if fully set forth). Due to the high level of component detail provided by NREL for both the MTG unit and the subsequent gasoline product separation units, the composition of the MTG reactor used in this study is based on the NREL report. The MTG unit will operate adiabatically at a temperature of 400° C. and 12.8 bar. The methanol feed will be pumped to 14.5 bar and heated to 330° C. for input to the reactor. The methanol will be converted to 44 wt % water and 56 wt % crude hydrocarbons, of which 2 wt % will be light gas, 19 wt % will be C₃-C₄ gases, and 19 wt % will be C₅₊ gasoline. The crude hydrocarbons will ultimately be separated into finished fuel products, of which 82 wt % will be gasoline, 10 wt. % will be LPG, and the balance will be recycle gases. This is modeled mathematically in the process synthesis model using an atom balance around the MTG unit and assuming a 100% conversion of the methanol entering the MTG reactor.

Any methanol entering the MTO process unit is heated to 400° C. at 1.2 bar. The MTO fluidized bed reactor operates at a temperature of 482° C. and a pressure of 1 bar. The exothermic heat of reaction within the MTO unit is controlled through generation of low-pressure steam. One hundred percent of the input methanol is converted into olefin effluent containing 1.4 wt % CH₄, 6.5 wt % C₂-C₄ paraffins, 56.4 wt % C₂-C₄ olefins, and 35.7 wt % C₅-C₁₁ gasoline. The MTO unit is modeled mathematically using an atom balance and a typical composition seen in the literature. The MTO product is fractionated (MTO-F) to separate the light gases, olefins, and gasoline fractions. The MTO-F unit is assumed to operate as a separator unit where 100% of the C₁-C₃ paraffins are recycled back to the refinery, 100% of the C₄ paraffins and 100% of the olefins are directed to the MOGD unit, 100% of the gasoline is combined with the remainder of the gasoline generated in the process, and 100% of the watergenerated in the MTO unit is sent for wastewater treatment.

The separated olefins are sent to the MOGD unit where a fixed bed reactor is used to convert the olefins to gasoline and distillate over a ZSM-5 catalyst. The gasoline/distillate product ratios can range from 0.12 to >100, and the ratio chosen in this study was 0.12 to maximize the production of diesel. The MOGD unit operates at 400° C. and 1 bar and will utilize steam generation to remove the exothermic heat of reaction within the unit. The MOGD unit is modeled with an atom balance and will produce 82% distillate, 15% gasoline, and 3% light gases. The product will be fractionated (MTODF) to remove diesel and kerosene cuts from the gasoline and light gases. The MTODF unit will be modeled as a separator unit where 100% of the C11AC13 species are directed to the kerosene cut and 100% of the C_{14 +} species are directed to the diesel cut.

LPG-Gasoline Separation.

The LPG and gasoline generated from ZSM-5 conversion of the FT hydrocarbons or the methanol must be passed through a series of separation units to extract the LPG from the gasoline and alkylate any isobutane to a blending stock for the final gasoline pool. (FIG. 64). Light gases are initially removed via one of two knock-out units, and the crude hydrocarbons are passed through a de-ethanizer column, a stabilizer column, an absorber column, a splitter column, and an LPG alkylate splitter to separate the LPG from the gasoline fractions. Each of these units is modeled mathematically as a splitter unit where the split fraction of each species to an output stream is given by the information in the PFDs P850-A1501 and P850-A1502 from the NREL study (National Renewable Energy Laboratory, 2011, which is incorporated herein by reference as if fully set forth). All low-pressure steam and cooling water needed for each of the units is derived for each of the units in the NREL study. The total amount of process utility that is needed per unit flow rate from the top or bottom of the column is calculated, and this ratio is used as a parameter in the process synthesis model to determine the actual amount of each utility needed based on the unit flow rate.

In addition to the distillation columns within this section, there is also an alkylation unit that is used to convert isobutane and butene to an alkylate blending stock for the gasoline pool. The alkylate was modeled as isobutane, and the alkylation unit was modeled using a species balance where the key species, butene, was completely converted to isobutane. Butene is used as the limiting species in this reaction, because it is generally present in a far smaller concentration than isobutane.

### Example 4.4

### Hydrogen/Oxygen Production

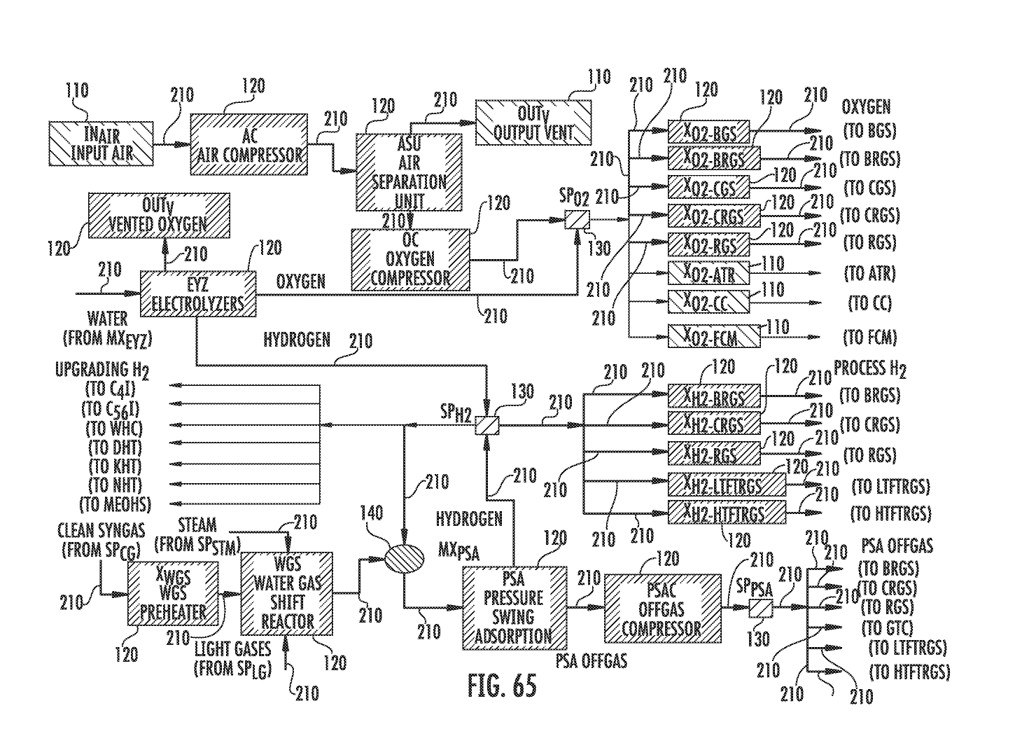

Hydrogen is produced via pressure-swing adsorption or an electrolyzer unit, whereas oxygen can be provided by the electrolyzer or a separate air separation unit (FIG. 65).

### Example 4.5

### Wastewater Treatment

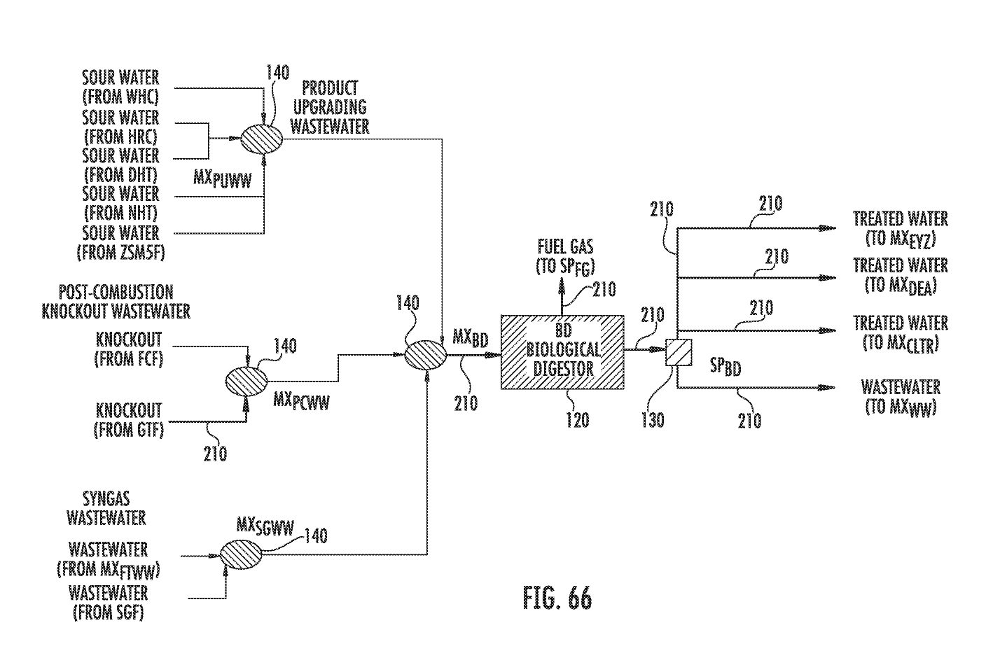

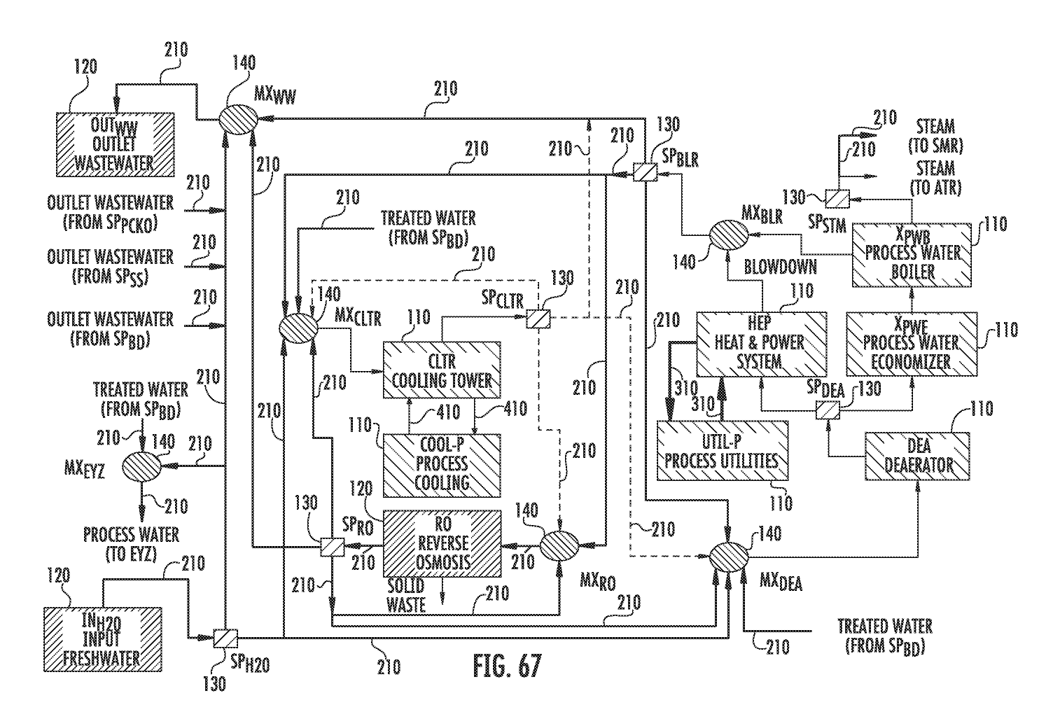

A complete wastewater treatment network (FIGS. 66 and 67) is incorporated that will treat and recycle wastewater from various process units, blowdown from the cooling tower, blowdown from the boilers, and input freshwater. Process wastewater is treated using only a biological digestor due to the negligible quantities of sulfur (e.g., H₂S) or nitrogen (e.g., NH₃) that are expected to be in the wastewater streams. Clean output of the network includes (1) process water to the electrolyzers, (2) steam to the autothermal reformer, steam reformer, and WGS reactor, and (3) discharged wastewater to the environment.

### Example 4.6

### Unit Costs

The total direct costs, TDC, for the GTL refinery hydrocarbon production and upgrading units are calculated using estimates from several literature sources using the cost parameters in Table 51 and Eq. 420

$$TDC = ( 1 + BOP ) \cdot {C}_{o} \cdot \frac{{S}_{r}^{sf}}{{S}_{o}} \qquad ( 420 )$$

where Cₒ is the installed unit cost, Sₒ is the base capacity, Sᵣ is the actual capacity, sf is the cost scaling factor, and BOP is the balance of plant percentage (site preparation, utility plants, etc.). The BOP is estimated to be 20% of the total installed unit cost. All capital cost numbers are converted to 2011 dollars using the Chemical Engineering Plant Cost Index.76 The cost estimates for the four natural gas conversion technologies are included in Table 51. Cost estimates for all other process units in the GTL refinery are taken from previous works and are included in Example 4.

TABLE 51
GTL refinery wastewater treatment reference
capacities, costs (2011$), and scaling factors

| Description | Cₒ (MM $) | Sₒ | S_{Max} | Units | Scale Basis | sf | Ref. |
|---|---|---|---|---|---|---|---|
| Autothermal reformer | 10.26 | 12.2 | 35.0 | kg/s | Natural gas feed | 0.67 | 71 |
| Steam-methane reformer | 63.74 | 26.1 | 35.0 | kg/s | Natural gas feed | 0.67 | 57 |
| Partial oxidation reactor | 650.1 | 118.8 | 75.0 | kg/s | Natural gas feed | 0.67 | 65 |
| OC reactor | 287.62 | 661.9 | 75.0 | kg/s | Natural gas feed | 0.67 | 65 |

The total plant cost, TPC, for each unit is calculated as the sum of the total direct capital, TDC, plus the indirect costs, IC. The IC include engineering, startup, spares, royalties, and contingencies and is estimated to 32% of the TDC. The TPC for each unit must be converted to a levelized cost to compare with the variable feedstock and operational costs for the process. Using the methodology of Kreutz et al., 2008, which is incorporated herein by reference as if fully set forth, the capital charges (CC) for the refinery are calculated by multiplying the levelized capital charge rate (LCCR) and the interest during construction factor (IDCF) by the total overnight capital (Eq. (421)).

CC=LCCR×IDCF×TPC (421)

Kreutz et al. 2008, which is incorporated herein by reference as if fully set forth, calculates an LCCR value of 14.38%/yr and an IDCF of 7.6%. Thus, a multiplier of 15.41%/yr is used to convert the TPC into a capital charge rate. Assuming an operating capacity (CAP) of 330 days/yr and operation maintenance (OM) costs equal to 5% of the TPC, the total levelized cost (Cost^{U}) associated with a unit is given by Eq. (422).

$${Cost}_{u}^{U} = ( \frac{LCCR \cdot IDCF}{CAP} + \frac{OM}{365} ) \cdot ( \frac{{TPC}_{u}}{Prod} ) \qquad ( 422 )$$

The levelized costs for the units described for natural gas conversion are added to the complete list of GTL process units in previous studies.

### Example 4.7

### Objective Function

The objective function for the model s given by Eq. (423). The summation represents the total cost of liquid fuels production and includes contributions from the feedstocks cost for natural gas (Cost_{NG}^{F}), freshwater (Cost_{H2O}^{F}), and butanes (Cost_{BUT}^{F}), the electricity cost (Cost^{El}), the CO₂ transportation, storage, and monitoring cost (Cost^{Seq}), and the levelized unit investment cost (Cost^{U}). Each of the terms in Eq. (423) is normalized to the total volume of products produced (Prod). Note that other normalization factors (e.g., total volume of gasoline equivalent and total energy of products) and other objective functions (e.g., maximizing the net present value) can be easily incorporated into the model framework.

$$MIN {Cost}_{NG}^{F} + {Cost}_{{H}_{2} O}^{F} + {Cost}_{BUT}^{F} + {Cost}^{EI} + {Cost}^{Seq} + {\sum }_{u ∈ {U}_{Inv}} {Cost}_{u}^{U} \qquad ( 423 )$$

The process synthesis model with simultaneous heat, power, and water integration represents a large-scale nonconvex mixed-integer nonlinear optimization model that was solved to global optimality using a branch-and-bound global optimization framework. At each node in the branch- and bound tree, a mixed-integer linear relaxation of the mathematical model is solved using CPLEX, and then, the node is branched to create two children nodes. The solution pool feature of CPLEX is utilized during the solution of the relaxed model to generate a set of distinct points (150 for the root node and 10 for all other nodes), each of which is used as a candidate starting point to solve the original model. For each starting point, the current binary variable values are fixed, and the resulting NLP is minimized using CONOPT. If the solution to the NLP is less than the current upper bound, then the upper bound is replaced with the NLP solution value. At each step, all nodes that have a lower bound that is within an e tolerance of the current upper bound

$$( \frac{{LB}_{node}}{UB} \geq 1 - ɛ )$$

are eliminated from the tree.

Computational Studies

The process synthesis model (see Example 3.16 and Example 4.15) was used to analyze 24 distinct case studies using an average representation of natural gas feedstock (Table 48). The global optimization framework was terminated, if all nodes in the branch-and-bound tree were processed or if 100 CPU hours had passed. The case studies were chosen to examine the effect of (1) plant capacity, (2) product composition, (3) natural gas conversion technology, and (4) GHG reduction requirement on the overall cost of fuel production and the optimal process topology. Four representative capacities of 1, 10, 50, and 200 kBD were chosen to examine the potential effect of economy of scale. The capacity of the plants is defined as “barrels per stream day,” which is computed by dividing the total number of produced barrels by the actual number of days that the GTL refinery was operational. All of the units are, therefore, appropriately sized to a “barrels per calendar day” figure using the capacity factor of the refinery (Eq. (422)). Liquid fuel (i.e., gasoline, diesel, and kerosene) production was selected to either (a) represent the 2010 United States demand (i.e., 67 vol % gasoline, 22 vol % diesel, and 11 vol % kerosene),84 (b) maximize the diesel production (i.e., >75 vol %), (c) maximize the kerosene production (i.e., >70 vol. %), or (d) freely output any unrestricted composition of the products. These case studies will be labeled as N-C, where N represents the type of product composition (i.e., R: 2010 U.S. ratios, D: max diesel, K: max kerosene, and U: unrestricted composition) and C represents the capacity in kBD. For example, the U-1 label represents the 1 kBD capacity refinery with an unrestricted product composition.

A second set of case studies will examine the effects of the natural gas conversion technology on the U-1 refinery. In each case study, the natural gas conversion technology will be fixed to either ATR, steam reforming, partial oxidation to methanol, or OC to olefins. These studies will be labeled as G-U-1, where G represents the type of natural gas conversion technology (i.e., A: ATR, S: steam reforming, P: partial oxidation, and C: OC). Each of the 20 case studies described earlier will ensure that the life cycle GHG emissions from the refinery are at most equal to current fossil-fuel-based processes. That is, the life cycle GHG emissions must be at most equal to that of a petroleum-based refinery (91.6 kg CO^{2eq}/GJ^{LHV}) for the liquid fuels or that of a natural gas combined cycle plant (101.3 kg CO^{2eq}/GJ) for electricity. The final four case studies will examine the effect of the utilization of CO₂ capture and sequestration on all vented streams from the refineries with an unrestricted product composition. For each of the four refinery capacities, a maximum of 1% of the input carbon will be allowed to be vented to the atmosphere as CO₂. The balance of the carbon must be contained within the liquid fuels or in CO₂ that is compressed and then sequestered. For each capacity, C, the case study will be labeled as U-C-Z.

The cost parameters used for the GTL refinery are listed in Table 52. The costs for feedstocks (i.e., natural gas, freshwater, and butanes) include all costs associated with delivery to the plant gate. The products (i.e., electricity and propane) are assumed to be sold from the plant gate and do not include the costs expected for transport to the end consumer. The cost of CO₂ capture and compression is included in the investment cost of the GTL refinery, whereas the cost for transportation, storage, and monitoring of the CO₂ is shown in Table 52.

TABLE 52
Cost parameters (2011$) for the CBGTL refinery

| Item | Cost | Item | Cost |
|---|---|---|---|
| Natural gas | $5/TSCF^{a} | Freshwater | $0.50/metric ton |
| Butanes | $1.84/gallon | Propanes | $1.78/gallon |
| Electricity | $0.07/kW h | CO₂ TS&M^{b} | $5/metric ton |

^{a}TSCF—thousand standard cubic feet.
^{b}TS&M—transportation, shipping, and monitoring.

Once the global optimization algorithm has completed, the resulting process topology provides (1) the operating conditions and working fluid flow rates of the heat engines, (2) the amount of electricity produced by the heat engines, (3) the amount of cooling water needed for the engines, and (4) the location of the pinch points denoting the distinct subnetworks. Given this information, the minimum number of heat exchanger matches necessary to meet specifications (1)-(4) are calculated as previously described. On solution of the minimum matches model, the heat exchanger topology with the minimum annualized cost can be found using the superstructure methodology. The investment cost of the heat exchangers is added to the investment cost calculated within the process synthesis model to obtain the final investment cost for the superstructure.

### Example 4.9

### Optimal Process Topologies

Information about the optimal process topologies for all case studies is shown in Table 53. For natural gas conversion (NG conv.), the possible choices are steam reforming (SMR), ATR, partial oxidation to methanol (PO), and OC. Three possible temperature options were used for the steam reformer (700, 800, and 900° C.), the autothermal reformer (800, 900, and 1000° C.), and the reverse WGS unit (400, 500, and 600° C.). For the 20 case studies that did not constrain the natural go s conversion technology, either the steam reformer or the autothermal reformer was selected as the optimal unit. Additionally, the operating temperatures of these units were consistently chosen to be at the upper operating limit (900° C. for SMR and 1000° C. for ATR). The choice of operating temperature within the reformers represents a balance among (1) the level of input steam needed, (2) the extent of consumption of CO₂ via the reverse WGS reaction, (3) the extent of methane conversion, and (4) the fuel gas or oxygen requirement to provide process heating. Lower reformer temperatures will have less favorable conditions for methane conversion and CO₂ consumption due to lower values of the equilibrium constants in the reformer. Alternatively, both the steam and the heating requirement will be smaller, decreasing the operating costs of the unit. Higher temperatures will have higher conversions of methane and CO₂ with a correspondingly higher steam and heating requirement. Selection of the high-temperature units shows that a key topological decision is the conversion of methane and CO₂ in the reformers. The decrease in the capital requirement of the downstream process units outweights the increased operating costs with a higher temperature.

TABLE 53
Topological information for the optimal solutions for the 24 case studies

| | Case Study | | | | | | | | | | | |
|---|---|---|---|---|---|---|---|---|---|---|---|---|
| | U-1 | U-10 | U-50 | U-200 | D-1 | D-10 | D-50 | D-200 | K-1 | K-10 | K-50 | K-200 |
| NG conv. | SMR | SMR | ATR | ATR | SMR | SMR | ATR | ATR | SMR | SMR | ATR | ATR |
| NG conv. temp. | 900 | 900 | 1000 | 1000 | 900 | 900 | 1000 | 1000 | 900 | 900 | 1000 | 1000 |
| WGS/RGS temp. | — | — | — | — | — | — | — | — | — | — | — | — |
| Min Wax FT | — | — | — | — | — | — | — | — | — | — | — | — |
| Nom. Wax FT | — | — | — | — | — | — | — | — | lr. rWGS | lr. rWGS | lr. rWGS | lr. rWGS |
| FT upgrading | — | — | — | — | — | — | — | — | Fract. | Fract. | Fract. | Fract. |
| MTG usage | Y | Y | Y | Y | — | — | — | — | — | — | — | — |
| MTOD usage | — | — | — | — | Y | Y | Y | Y | — | — | — | — |
| CO2SEQ usage | Y | Y | Y | Y | Y | Y | Y | Y | Y | Y | Y | Y |
| GT usage | — | — | — | — | — | — | — | — | — | — | — | — |
| | R-1 | R-10 | R-50 | R-200 | A-U-1 | S-U-1 | P-U-1 | C-U-1 | U-1-Z | U-10-Z | U-50-Z | U-200-Z |
| NG conv. | SMR | SMR | ATR | ATR | ATR | SMR | PO | OC | SMR | SMR | ATR | ATR |
| NG conv. temp. | 900 | 900 | 1000 | 1000 | 1000 | 950 | 450 | 800 | 900 | 900 | 1000 | 1000 |
| WGS/RGS temp. | — | — | — | — | — | — | — | — | — | — | — | — |
| Min Wax FT | — | — | — | — | — | — | — | — | — | — | — | — |
| Nom. Wax FT | — | lr. rWGS | lr. rWGS | lr. rWGS | — | — | — | — | — | — | — | — |
| FT upgrading | — | ZSM-5 | ZSM-5 | ZSM-5 | — | — | — | — | — | — | — | — |
| MTG usage | Y | Y | Y | Y | Y | Y | Y | — | Y | Y | Y | Y |
| MTOD usage | Y | — | — | — | — | — | — | — | — | — | — | — |
| CO2SEQ usage | Y | Y | Y | Y | Y | Y | Y | Y | Y | Y | Y | Y |
| GT usage | — | — | — | — | — | — | — | — | — | — | — | — |

The temperature of the conversion technology is selected along with the operating temperature of the reverse WGS unit (RGS), if utilized. The presence of a CO₂ sequestration system (CO2SEQ) or a GT is noted using yes (Y) or no (N). The minimum wax and maximum wax FT units are designated as either cobalt-based or iron-based units. The iron-based units will either facilitate the forward (fWGS) or reverse water gas-shift (rWGS) reaction. The FT vapor effluent will be upgraded using fractionation into distillate and naphtha (Fract.) or ZSM-5 catalytic conversion. The use of MTG and MTO/MOGD is noted using yes (Y) or no (2).

Selection of a specific reformer to convert the natural gas is critical for two major reasons. First, though the cost of a steam reformer is higher than the autothermal reformer (Table 51), the additional cost of air separation to produce high-purity oxygen makes the autothermal reformer a more capital intensive choice to produce synthesis gas at lower capacity levels. However, as the refinery capacity increases, there is a definable point where the capital and operating costs of steam reforming are greater than the sum of ATR and air separation. An insight can be found by observing that the scaling factor of the reforming units is assumed to by 0.67 (Table 51), whereas that of the air separation unit is assumed to be 0.5 based on the study by Kreutz et al., 2008, which is incorporated herein by reference as if fully set forth, (Table 60). At some critical capacity level, the capital cost of the air separation unit and the autothermal reformer will be equal to that of a steam reformer, and it is anticipated that higher capacity levels will favor ATR, whereas lower capacity levels favor steam reforming. This is evident when comparing case studies A-U-1 and S-U-1. Table 55 shows that the use of an autothermal reformer adds about 5% to the investment cost of the plant and ultimately increases the cost of liquid fuels by 7%.

Second, the use of an autothermal reformer will generally require CO₂ removal prior to entry into a methanol or FT synthesis unit. The relative ratio of H₂ to CO or CO₂ exiting the autothermal reformer is less than the ideal stoichiometric ratio, so the synthesis gas composition must be adjusted appropriately via addition of H₂ or removal of CO₂. This is readily accomplished through the use of industrially commercialized precombustion CO₂ capture technology,75 which can provide recycle of the CO₂ to the autothermal reformer. The extent of CO₂ recycle vs. venting or sequestration is dependent on the level of heat integration within the plant and the life cycle GHG emissions requirement. Conversely, the steam reformer will generally output a synthesis gas that has too high of a H₂ content (e.g., >5). CO₂ recycle to the reformer can also be utilized to reduce the concentration of H₂ and increase the overall carbon efficiency of the plant. However, the CO₂ will need to be recovered from an atmospheric pressure flue gas stream using postcombustion capture technology that is not as commercially prevalent as the precombustion capture technology and will require a higher level of process contingency.

For the autothermal reformer, any H₂ addition must be from a noncarbon-based source (i.e., electrolyzers), because the production of H₂ from natural gas will effectively decrease the overall carbon conversion yield of the process and increase the level of GHG emissions. If electrolyzers were used to produce additional H₂, note that they will also be able to provide the O₂ for the autothermal reformer and eliminate the need for an air separation unit. However, the high capital and operating costs of electrolysis generally prevent these units from being an economically competitive Option. Factors that could positively impact the use of electrolyzers include (1) reducing the capital cost, (2) increasing efficiency, (3) the resale value of excess H₂ or O₂, and (4) the market value of electricity.

None of the 24 case studies utilized a dedicated reverse WGS unit for CO₂ consumption. The equilibrium constant for the WGS reaction at the expected operating temperatures of the dedicated unit make for less favorable conditions than the operating temperatures of the steam reformer or autothermal reformer. Therefore, in the 22 case studies that used a reformer to convert natural gas to synthesis gas, a portion of the CO₂ that was captured from the GTL refinery was directed to the reformer for consumption. In each of the 22 case studies, CO₂ consumption also occurred in the FT or methanol synthesis units. The reverse WGS reaction was able to occur at these lower temperatures due to the consumption of CO for the synthesis reactions. This decrease of CO provides the key driver for the consumption of CO₂ that is otherwise unavailable in a dedicated reverse WGS unit.

The 12 case studies that allowed for an unrestricted liquid product composition all selected methanol synthesis and MTG as the optimal technology. This reflects the expected reduction in capital costs associated with hydrocarbon production via methanol synthesis vs. FT synthesis that come from the reduced capital cost of methanol synthesis and MTG. Note that gasoline can be produced from FT synthesis and subsequent conversion of the hydrocarbons to gasoline via a ZSM-5 catalyst, but this process requires a higher capital investment over methanol synthesis. Both the MTG and the FT/ZSM-5 processes will produce a significant amount of byproduct LPG (9 vol %). The four case studies that maximize the diesel production utilized the methanol-to-olefins (MTO) and the MOGD processes to produce a high-quality diesel, whereas the four case studies that maximize kerosene will use a iron-based low-temperature FT synthesis followed by standard fractionation of the hydrocarbon species. The four case studies that produce liquid fuels in the ratios consistent with United States demands show a significant topological trade-off at different capacity levels. That is, at the 1 kBD capacity, methanol synthesis and subsequent conversion is the sole method for producing liquid fuels. As the capacity of the GTL refinery increases, the iron-based low-temperature FT unit is incorporated to provide the distillate products via wax hydrocracking and the gasoline product through ZSM-5 conversion. Additional gasoline is produced via the MTG route to provide the balance of the plant requirement.

In all 24 cases, CO₂ sequestration was utilized to provide a reduction in life cycle emissions for the GTL refinery. The first 20 case studies only incorporate CO₂ sequestration for a portion of the produced CO₂, while the balance of the CO₂ is either recycled back to the process or vented. In these cases, the cost of CO₂ capture may be required to meet process operating conditions or economically justified to increase the carbon yield of the process. CO₂ sequestration is solely utilized as a basis for GHG reduction and does not provide any economic benefit to the GTL refinery if a CO₂ tax is not imposed on the process. The final four case studies (U-C-Z) show the effect of forcing a maximum on the vented CO₂. The topological design of the units to produce the liquid fuels is equivalent to the corresponding case studies that do not impose the upper limit on the CO₂ venting (i.e., U-C). The only additions that are included in these last four case studies are the additional CO₂ capture/sequestration capacity and the resulting increase in the capital cost and utility requirement of the plant. For all case studies, waste heat is converted to steam for use both in the process units and in the steam cycle to provide electricity. G-Ts were not selected for use in any of the studies.

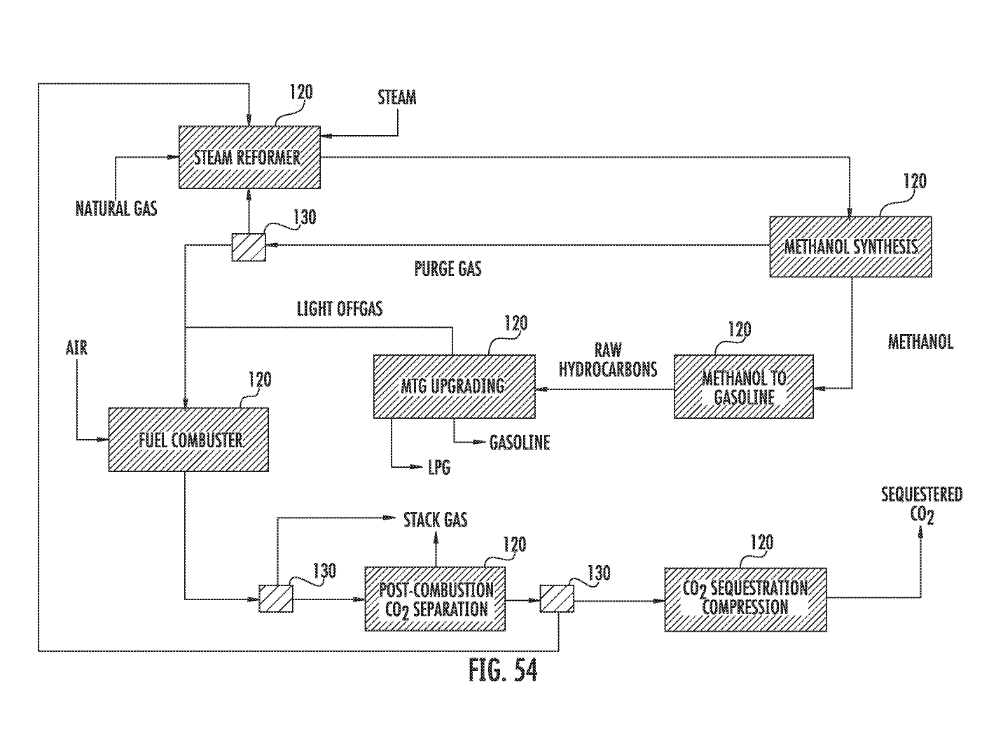

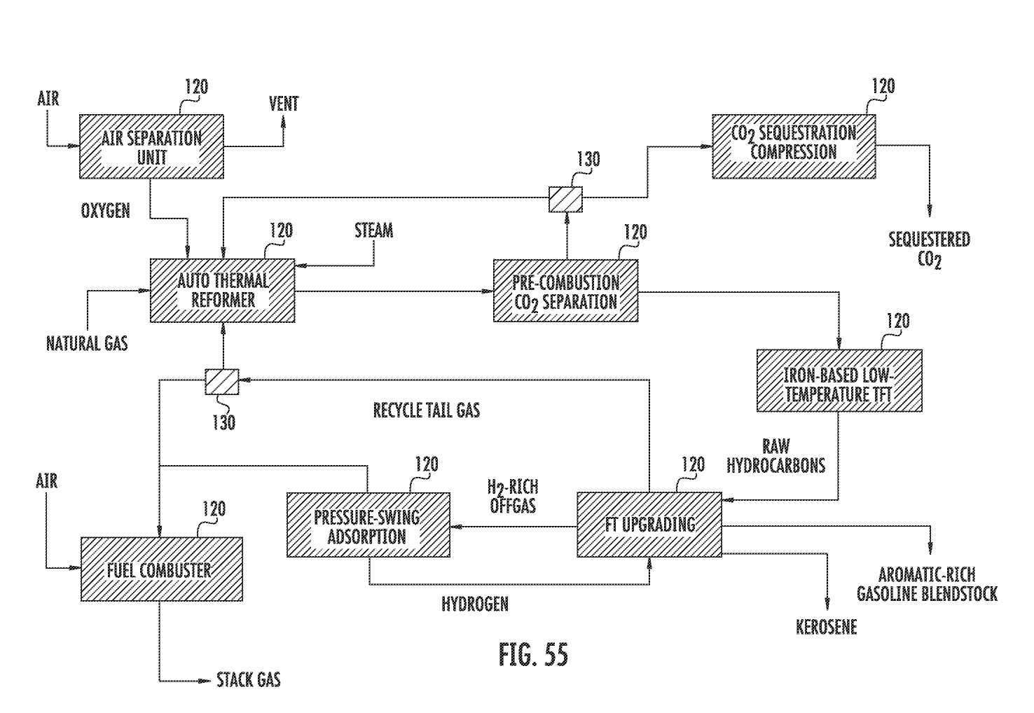

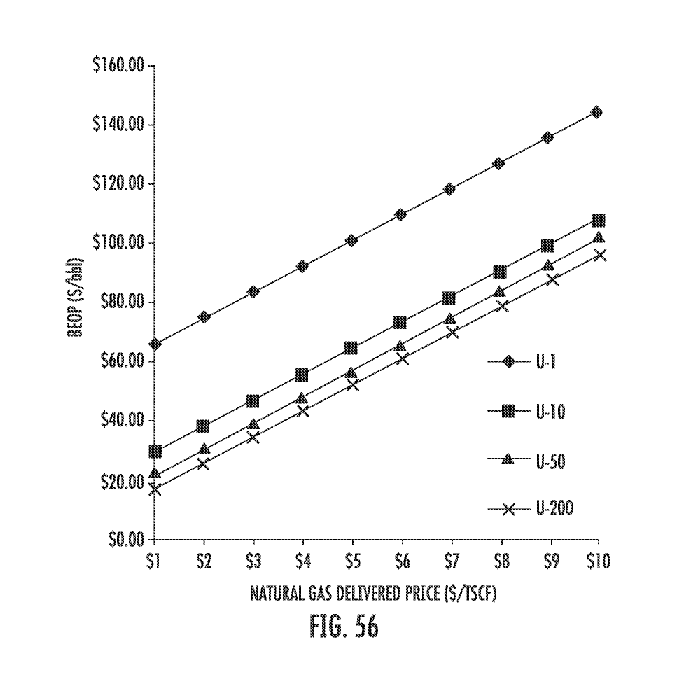

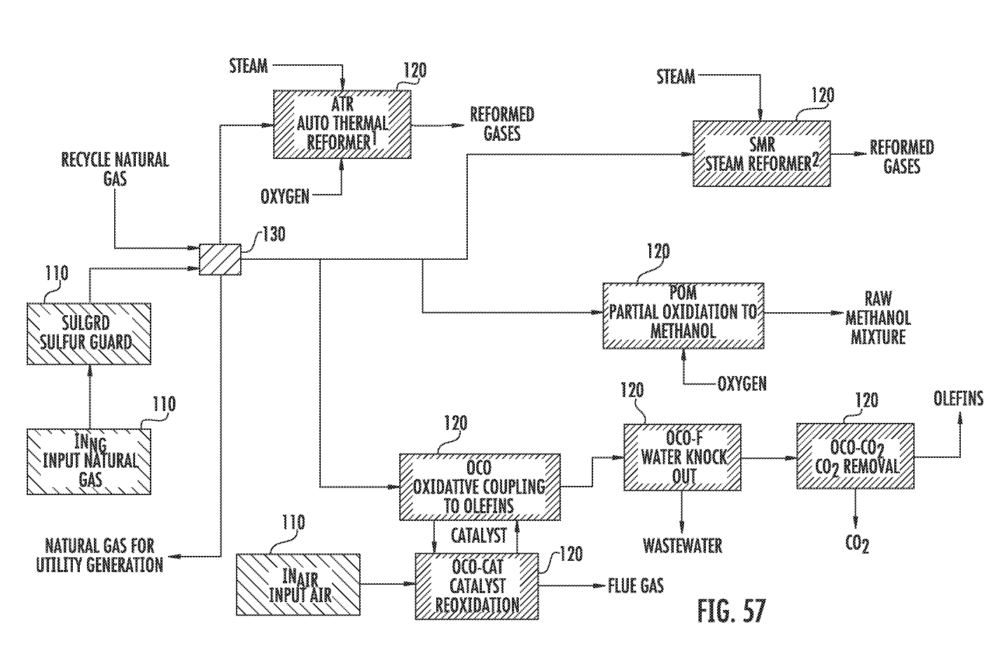

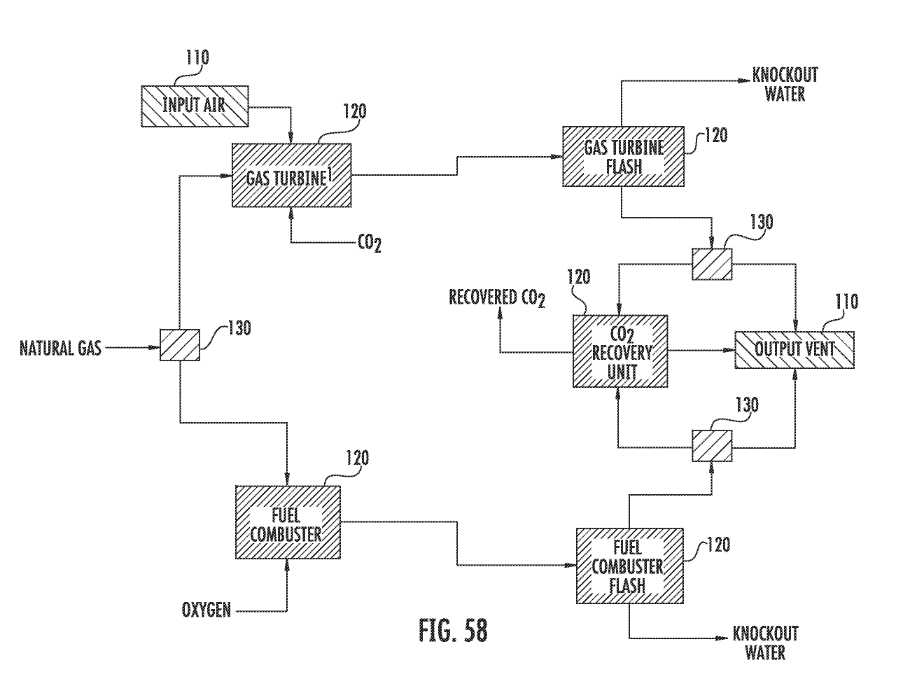

As an illustrative example, PFDs for the U-1 and the K-50 case studies are shown in FIGS. 54 and 55. These PFDs highlight the key points for natural gas conversion, hydrocarbon conversion, hydrocarbon upgrading, and CO₂ handling that are implemented in each of the 24 case studies. Note that several process units including heat exchangers, compressors, flash units, distillation columns, and turbines are not shown. The PFD for U-1 shows the natural gas conversion through steam reforming with recycle CO₂ being provided by postcombustion separation. Note that only a portion of the flue gas from the combustor is passed through the CO₂ separation unit, while the balance is sent to the stack. This split fraction is chosen so as to only capture the CO₂ that needs to be recycled or sequestered. All additional CO₂ that will be vented will simply bypass the postcombustion capture unit and flow to the stack. The heat needed for the steam reforming reaction is provided by recycle fuel gas passing over the fuel combustor unit. Therefore, no additional natural gas input is needed to provide the heat for steam reforming. The syngas exiting the steam reformer passes through the methanol synthesis section where recycle of the unreacted syngas yields an overall conversion of 94% of the CO and CO₂ to methanol. The methanol is then converted to raw hydrocarbons via a ZSM-5 catalyst, which are separated and upgraded to gasoline and LPG. All additional case studies that utilize steam reforming will implement a natural gas conversion and CO₂ handling section that is very similar to that of FIG. 54. The key differences in the PFDs are found in the hydrocarbon conversion and hydrocarbon upgrading sections, which are chosen based on the composition of fuels that is desired from the plant.

The PFD for the K-50 case study (FIG. 52) highlights an important difference in the natural gas conversion and the CO₂ handling associated with ATR. Specifically, the input natural gas is converted to syngas using steam and oxygen provided by the air separation unit. Precombustion capture technology is then used on the entire syngas steam to remove the CO₂ for recycle or sequestration. The resulting syngas that exits the CO₂ capture unit will, therefore, have a low CO₂ concentration, and an H₂-to-CO ratio of about 2. In this case study, the syngas is converted to raw hydrocarbons via the low-temperature FT reactor, which provides a significant quantity of wax that is an ideal feedstock for distillate production. Reforming of the naphtha fraction from the FT unit will provide an aromatic-rich gasoline blendstock and an H₂-rich offgas stream. Pure H₂ that is needed for hydrocracking and hydrotreating is extracted from the H₂-rich offgas via pressure-swing adsorption. Unreacted syngas from the FT reactor is mostly recycled to the ATR unit with a portion passing over the fuel combustor to provide heat for the refinery. Postcombustion capture of the CO₂ in the stack gas is not utilized. The natural gas conversion and CO₂ handling are similar for all case studies that utilize the ATR for syngas production.

### Example 4.10

### Overall Costs of Liquid Fuels

The overall cost of liquid fuel production (in $/GJ) is based on the costs of feedstocks, capital investment, operation and maintenance, and CO₂ sequestration and can be partially defrayed using byproduct sales of LPG and electricity. Feedstock costs are based on the as-delivered price for natural gas, butanes needed for the isomerization process, and freshwater needed to make up for process losses. Table 57 outlines the breakdown of the cost contribution for each case study, as well as the lower bound and the optimality gap values. The total cost is also converted into a break-even oil price (BEOP) in $/barrel (bbl) based on the refiner's margin for gasoline, diesel, or kerosene and represents the price of crude oil at which the GTL process becomes economically competitive with petroleum-based processes. The lower bound found by the global optimization framework is reported along with the corresponding optimality gap that ranges between 3 and 6% for each of the case studies.

The BEOP ranges between $101/bbl and $122/bbl for a 1 kBD plant, $64/bbl and $76/bbl for a 10 kBD plant, $57/bbl and $69/bbl for a 50 kBD plant, and $52/bbl and $64/bbl for a 200 kBD plant. The two major components that contribute to the overall cost are the natural gas feedstock and the costs related to capital investment (i.e., capital charges, operation, and maintenance). There is a significant economy of scale that is expected when increasing the plant capacity from 1 to 10 kBD, because a singular train (i.e., parallel combination of units) will be needed for most sections of the plant. That is, only one natural gas conversion unit (steam reformer or direct conversion), FT synthesis, methanol synthesis, or methanol conversion unit will be needed to produce the given quantity of liquid fuels. Once the capacity of the plant rises to 50 or 200 kBD, several trains will be required throughout the GTL refinery to process the large quantities of material in the plant. Some capital cost savings may be expected, because multiple units in the same train may share some auxiliary equipment, and the labor required to install the units is generally less than a linear increase. However, the effect of economy of scale will be diminished for GTL plants above 10-20 kBD.

For a given capacity, Table 54 shows that the overall fuels cost will depend on the type/composition of liquid fuels produced. The unrestricted composition cases (U) tend to have the lowest overall fuels cost, followed by the max diesel cases, then the max kerosene cases, and finally the United States ratio cases. The change in the BEOP is primarily due to the change in the investment cost between these groups of case studies, which is a function of the GTL refinery complexity that is needed to produce the desired liquid fuels. For the unrestricted case studies, the sale of byproduct LPG is assumed to provide a stronger economic benefit than the other case studies. If the production of LPG from the MTG technology is not desired, the LPG may be consumed in the process to produce synthesis gas via steam reforming or ATR or converted to C61 aromatics via the Cyclar process. The choice of technology will ultimately depend on the available market for LPG and aromatic chemicals or the aromatics requirements of the output gasoline.

Four case studies that enforce near-zero levels of CO₂ venting show the increase in the BEOP as a result of additional CO₂ capture/sequestration installed capacity. An increase of 5-8% in the overall cost is seen over the U-C case studies, which is partially due to the increase in investment cost and a decrease in the sale of byproduct electricity. The four case studies that enforce one particular type of natural gas conversion technology show that the natural gas direct conversion case studies are less economically attractive than the reforming cases. This is consistent with earlier studies of direct conversion technologies, which are limited by the low conversion of methane that is typically allowed in these processes. Improvements in the methanol yield from partial oxidation or olefins content from OC may reduce the capital investment associated with these processes to a point where it is favorable with the indirect conversion technologies. The overall cost results are included for six additional runs where either the autothermal reformer or steam reformer was fixed as the natural gas conversion technology. Runs A-U-C show how the BEOP changes for the autothermal reactor cases as capacity increases, whereas runs S-U-C show similar results for the steam reformer. For the 1 and 10 kBD case studies, the steam reformer provides a less expensive means for fuel production, whereas the autothermal reformer is more economical at 50 and 200 kBD. This is largely due to increased investment and natural gas costs associated with the autothermal reformer at low capacities and the steam reformer at higher capacities.

Parametric Analysis

Table 54 indicates that the two largest contributions to the overall fuels cost are the fixed/variable capital costs (i.e., capital charges and operation/maintenance) and the natural gas purchase cost. The case studies outlined above have assumed that natural gas is available at the national average price, though this may be higher or lower throughout the country depending on the location, availability, and demand for the feedstock. Therefore, it is important to investigate how the BEOP will be effected for changing purchase costs of natural gas. As an illustrative example, the BEOP for the U-C case studies is calculated, assuming that natural gas is priced from $1/thousand standard cubic feet (TSCF) to $10/TSCF. Note that the resale value of electricity may be directly tied to the purchase price of natural gas, so the price of electricity should change accordingly with the natural gas price. Assuming that the natural gas cost is 80% of the price of electricity, then the electricity will change linearly between $0.025/kW h and $0.126/kW h, as the natural gas price increases.

The resulting parametric analysis is plotted in FIG. 53. For the 1 kBD case study, the BEOP ranges from $70/bbl to $143/bbl at the natural gas price increases from $1/TSCF to $10/TSCF. The range of BEOP for the 10 kBD case is $33-105/bbl, $26-99/bbl for the 50 kBD case, and $20-94/bbl for the 200 kBD case. This analysis highlights the key economic advantages with the development of a refinery in a location with a low delivered cost of natural gas (e.g., $1/TSCF-$3/TSCF). Lower costs of natural gas allow for the small capacity processes outlined in this study to be constructed with significantly less economic risk. Note that the effect of changing the natural gas purchase price will be similar for other case studies with a similar capacity.

In addition, the capital costs of the units may also vary geographically or over time, and there are uncertainties associated with the nominal capital costs used in this study. Investigating the capital cost effect for each unit on the optimal topology will require a large combination of parametric study. To address this, the process synthesis approach using optimization under uncertainty will be studied as a future subject. In this article, however, a uniform increase of 5% in the unit capital costs produces a 2-3.5% increase in the overall cost of fuel production for all case studies.

TABLE 54
Overall cost results for the 24 case studies

| Contribution to | | | | | | | | | | | | | | | | |
|---|---|---|---|---|---|---|---|---|---|---|---|---|---|---|---|---|
| Cost ($/GJ of | Case Study | | | | | | | | | | | | | | | |
| Products) | U-1 | U-10 | U-50 | U-200 | D-1 | D-10 | D-50 | D-200 | K-1 | K-10 | K-50 | K-200 | R-1 | R-10 | R-50 | R-200 |
| Natural Gas | 7.67 | 7.60 | 7.81 | 7.75 | 8.13 | 7.93 | 7.96 | 7.78 | 7.62 | 7.44 | 7.71 | 8.14 | 7.62 | 7.62 | 7.59 | 7.60 |
| Butane | — | — | — | — | — | — | — | — | — | — | — | — | 0.35 | 0.35 | 0.31 | 0.29 |
| Water | 0.03 | 0.02 | 0.02 | 0.03 | 0.03 | 0.02 | 0.03 | 0.03 | 0.02 | 0.03 | 0.02 | 0.02 | 0.02 | 0.02 | 0.02 | 0.03 |
| CO₂ TS&M | 0.03 | 0.02 | 0.04 | 0.04 | 0.05 | 0.03 | 0.04 | 0.03 | 0.03 | 0.02 | 0.03 | 0.06 | 0.04 | 0.04 | 0.05 | 0.05 |
| Investment | 11.88 | 6.86 | 5.77 | 5.02 | 11.94 | 6.88 | 5.67 | 4.89 | 12.30 | 6.98 | 5.84 | 5.04 | 12.49 | 7.17 | 6.07 | 5.31 |
| O&M | 3.14 | 1.81 | 1.52 | 1.33 | 3.15 | 1.82 | 1.50 | 1.29 | 3.25 | 1.84 | 1.54 | 1.33 | 3.30 | 1.89 | 1.60 | 1.40 |
| Electricity | −0.72 | −0.74 | −0.83 | −0.68 | −0.68 | −0.74 | −0.79 | −0.70 | −1.13 | −0.94 | −1.28 | −1.29 | −1.12 | −1.12 | −0.83 | −0.83 |
| LPG | −2.05 | −2.05 | −2.05 | −2.05 | −0.93 | −0.85 | −0.69 | −0.69 | — | — | — | — | −0.46 | −0.46 | −0.41 | −0.42 |
| Total ($GJ) | 19.97 | 13.53 | 12.27 | 11.44 | 21.68 | 15.10 | 13.72 | 12.63 | 22.08 | 15.38 | 13.86 | 13.30 | 22.24 | 15.52 | 14.39 | 13.43 |
| Total ($/bbl) | 101.03 | 64.31 | 57.16 | 52.38 | 110.77 | 73.25 | 65.38 | 59.20 | 113.06 | 74.87 | 66.21 | 63.03 | 114.00 | 75.68 | 69.23 | 63.76 |
| Lower Bound | 19.08 | 12.81 | 11.73 | 10.85 | 20.51 | 14.42 | 12.97 | 12.01 | 21.01 | 14.88 | 13.11 | 12.73 | 21.59 | 15.07 | 13.93 | 12.79 |
| ($/GJ) | | | | | | | | | | | | | | | | |
| Gap | 4.46% | 5.34% | 4.39% | 5.10% | 5.37% | 4.51% | 5.44% | 4.91% | 4.85% | 3.26% | 5.42% | 4.34% | 2.95% | 2.93% | 3.17% | 4.78% |
| | U-1-Z | U-10-Z | U-50-Z | U-200-Z | P-U-1 | C-U-1 | A-U-1 | S-U-1 | A-U-10 | S-U-10 | A-U-50 | S-U-50 | A-U-200 | S-U-200 | | |
| Natural Gas | 7.67 | 7.60 | 7.81 | 7.75 | 8.13 | 7.82 | 8.15 | 7.67 | 7.71 | 7.60 | 7.81 | 7.92 | 7.75 | 7.70 | | |
| Butane | — | — | — | — | — | — | — | — | — | — | — | — | — | — | | |
| Water | 0.03 | 0.02 | 0.02 | 0.03 | 0.02 | 0.02 | 0.03 | 0.03 | 0.02 | 0.02 | 0.02 | 0.02 | 0.03 | 0.03 | | |
| CO₂ TS&M | 0.03 | 0.02 | 0.04 | 0.04 | 0.06 | 0.04 | 0.07 | 0.03 | 0.02 | 0.02 | 0.04 | 0.04 | 0.04 | 0.04 | | |
| Investment | 12.29 | 7.02 | 5.88 | 5.33 | 13.74 | 14.73 | 12.43 | 11.88 | 6.99 | 6.86 | 5.77 | 5.86 | 5.02 | 5.35 | | |
| O&M | 3.25 | 1.86 | 1.55 | 1.41 | 3.63 | 3.89 | 3.28 | 3.14 | 1.85 | 1.81 | 1.52 | 1.55 | 1.33 | 1.41 | | |
| Electricity | −0.29 | −0.30 | −0.33 | −0.27 | −0.91 | −0.75 | −0.68 | −0.72 | −0.74 | −0.74 | −0.83 | −0.84 | −0.68 | −0.69 | | |
| LPG | −2.05 | −2.05 | −2.05 | −2.05 | −2.05 | −2.05 | −2.05 | −2.05 | −1.99 | −2.05 | −2.05 | −2.08 | −2.05 | −2.09 | | |
| Total ($GJ) | 20.92 | 14.18 | 12.92 | 12.22 | 22.62 | 23.70 | 21.22 | 19.97 | 13.86 | 13.53 | 12.27 | 12.46 | 11.44 | 11.74 | | |
| Total ($/bbl) | 106.46 | 68.00 | 60.84 | 56.88 | 116.13 | 122.30 | 108.15 | 101.03 | 66.22 | 64.31 | 57.16 | 58.22 | 52.38 | 54.13 | | |
| Lower Bound | 19.89 | 13.54 | 12.49 | 11.80 | 21.53 | 22.94 | 20.02 | 19.16 | 13.06 | 12.81 | 11.73 | 11.97 | 10.85 | 11.18 | | |
| ($/GJ) | | | | | | | | | | | | | | | | |
| Gap | 4.92% | 4.46% | 3.29% | 3.50% | 4.81% | 3.22% | 5.63% | 4.04% | 5.78% | 5.34% | 4.39% | 3.94% | 5.10% | 4.79% | | |

The contribution to the total costs (in $/GJ) come from natural gas, butanes, water, CO₂ transportation/storage/monitoring (CO₂ TS&M), investment, and operations/maintenance (O&M). Propane and electricity are sold as byproducts (negative value). The overall costs are reported in ($/GJ) and ($/bbl) basis, along with the lower bound values in ($/GJ) and the optimality gap between the reported solution and the lower bound.

### Example 4.12

### Investment Costs

The TPC is decomposed into cost contributions from different sections of the plant in Table 55, namely the syngas generation, syngas cleaning, hydrocarbon production, hydrocarbon upgrading, hydrogen/oxygen production, heat and power integration, and wastewater treatment sections. For the case studies that utilize indirect conversion of natural gas, the syngas generation section and the hydrocarbon production section are consistently the highest contributing factors in the investment cost. The cost of utility production (i.e., electricity and steam) generally make up the third most expensive component, with syngas cleaning (i.e., CO₂ capture and compression) and hydrocarbon upgrading following next. The values in Table 55 can be converted to a “total overnight cost” by adding the anticipated preproduction costs, inventory capital, financing costs, and other owner's costs and then to a “total as-spent capital” by figuring in capital escalation and interest on debt that occurs during construction. Note that this information has been accounted for when determining the capital charge factor to use for the GTL refinery.

The TPC ranges from $138 to $171 MM for 1 kBD plants, $798 to $834 MM for 10 kBD plants, $3354 to $3527 MM for 50 kBD plants, and $11,384 to $12,387 MM for 200 kBD plants. The normalized investment costs reveal the economies of scale obtained at the different capacity levels and range from $138 k to $171 k/bpd for 1 kBD, $80 k to $84 k/bpd for 10 kBD plants, $67 k to $70 k/bpd for 50 kBD plants, and $58 k to $62 k/bpd for 200 kBD plants. Among the case studies, the plants with an unrestricted fuel requirement and the max diesel cases both provide similar TPCs. The increased costs associated with hydrogen production and a more complicated hydrocarbon refining section for the max diesel cases are balanced by an overall increase in the gas capacity required in the unrestricted cases. The LPG produced in the refineries is not added to the total plant capacity, because this is not considered to be a liquid transportation fuel and is merely a byproduct. Therefore, the plants that utilize the MTG technology must have higher capacities for natural gas conversion and methanol synthesis, because ˜10% of the carbon in the process will leave as LPG. The increase in costs for the max kerosene and the United States ratio cases is mostly associated with the use of FT synthesis and the upgrading of the hydrocarbon products, though these two sets of case studies are typically 3-6% higher than the case studies that utilize methanol synthesis.

The driving factor for the selection of steam reforming or ATR of natural gas as the preferred route in all the case studies is most clearly illustrated in case studies A-U-1, S-U-1, P-U-1, and C-U-1, where the natural gas conversion technology is imposed in each case study. In Table 55, the case studies that utilize direct conversion of natural gas (i.e., P-U-1 and C-U-1) have higher hydrocarbon production/upgrading costs. For the OC case (i.e, C-U-1), the cost of olefins production is much higher than that for hydrocarbon production from the indirect cases due to low conversion rates of methane and the subsequent high costs of compression for the recycle gases. The units utilized in this topology include the olefin fractionation (MTO-F), olefins to gasoline to distillate (OGD), hydrocarbon fractionation (MTODF), distillate and kerosene hydrotreaters (DHT, KHT), and the units in the LPG-gasoline separation section (see FIGS. 50 and 51). The effect of the low conversion is the high flow rate of recycle gases in Examples 4, FIG. 64 that increase the volumetric flow rate for the CO₂ separation (Table 60) and compression to recycle the gases to various process units.

Similarly, the low selectivity of methanol in the partial oxidation of natural gas (case study P-U-1) has the same effect on the hydrocarbon production and upgrading costs. The offgas stream in FIG. 63 is high, increasing the capital cost of the subsequent units. For the case studies enforcing a CO₂ venting maximum, note that the majority of the cost increase is associated with the syngas cleaning as a result of additional CO₂ capture/compression capacity. In general, this results in an increase of about 5% to the TPC from the other unrestricted case studies (U-C).

TABLE 55
Breakdown of the investment costs for the 24 case studies

| | Case Study | | | | | | | | | | | |
|---|---|---|---|---|---|---|---|---|---|---|---|---|
| | U-1 | U-10 | U-50 | U-200 | D-1 | D-10 | D-50 | D-200 | K-1 | K-10 | K-50 | K-200 |
| Syngas generation | 53 | 318 | 1361 | 4439 | 48 | 288 | 1164 | 4152 | 54 | 287 | 1158 | 3736 |
| Syngas cleaning | 10 | 57 | 242 | 830 | 8 | 47 | 215 | 763 | 8 | 47 | 195 | 666 |
| Hydrocarbon production | 39 | 224 | 926 | 3450 | 39 | 215 | 907 | 2942 | 40 | 236 | 997 | 3583 |
| Hydrocarbon upgrading | 11 | 59 | 266 | 904 | 14 | 79 | 326 | 1190 | 13 | 74 | 336 | 1243 |
| Hydrogen/oxygen production | — | — | — | — | 8 | 48 | 190 | 634 | 8 | 47 | 210 | 685 |
| Heat and power integration | 19 | 112 | 443 | 1654 | 17 | 98 | 387 | 1311 | 16 | 95 | 384 | 1432 |
| Wastewater treatment | 5 | 28 | 116 | 408 | 4 | 25 | 110 | 392 | 5 | 25 | 113 | 387 |
| Total (MM $) | 138 | 798 | 3354 | 11685 | 139 | 800 | 3299 | 11384 | 143 | 812 | 3393 | 11732 |
| Total ($/bpd) | 138,127 | 79,808 | 67,072 | 58,427 | 138,799 | 79,997 | 65,977 | 56,922 | 412,978 | 81,192 | 67,866 | 58,658 |
| | R-1 | R-10 | R-50 | R-200 | A-U-1 | S-U-1 | P-U-1 | C-U-1 | U-1-Z | U-10-Z | U-50-Z | U-200-Z |
| Syngas generation | 49 | 278 | 1165 | 4067 | 50 | 53 | — | — | 53 | 308 | 1324 | 4589 |
| Syngas cleaning | 9 | 50 | 227 | 771 | 10 | 10 | 5 | 5 | 15 | 88 | 368 | 1202 |
| Hydrocarbon production | 40 | 232 | 995 | 3512 | 38 | 39 | 67 | 122 | 39 | 219 | 917 | 3558 |
| Hydrocarbon upgrading | 16 | 93 | 388 | 1259 | 11 | 11 | 56 | 23 | 11 | 60 | 260 | 927 |
| Hydrogen/oxygen production | 8 | 51 | 208 | 763 | 11 | — | 11 | — | — | — | — | — |
| Heat and power integration | 18 | 104 | 436 | 1628 | 19 | 19 | 17 | 17 | 19 | 114 | 438 | 1702 |
| Wastewater treatment | 5 | 26 | 107 | 348 | 5 | 5 | 4 | 5 | 5 | 28 | 114 | 408 |
| Total (MM $) | 145 | 834 | 3527 | 12,347 | 144 | 138 | 160 | 171 | 143 | 817 | 3420 | 12,387 |
| Total ($/bpd) | 145,227 | 83,393 | 70,538 | 61,736 | 144,488 | 138,127 | 159,744 | 171,327 | 142,900 | 81,657 | 68,408 | 61,933 |

The major sections of the plant include the syngas generation section, syngas cleaning, hydrocarbon production, hydrocarbon upgrading, hydrogen/oxygen production, heat and power integration, and wastewater treatment blocks. The values are reported in MM $ and normalized with the amount of fuels produced ($/bpd).

### Example 4.13

### Material and Energy Balances

The overall material and energy balances for the 24 case studies are shown in Tables 56 and 57, respectively. The natural gas is shown in million standard cubic feet per hour (mscf/h), whereas the butane, liquid products, and water are shown in kBD. For all the plants of a given capacity, a similar quantity of natural gas needed, which is consistent with the cost results in Table 54. The major differences between the case studies are based on the type and quantity of liquid fuels that are produced along with the amount of CO₂ that is sequestered and vented. For the unrestricted case studies, the refinery capacity is solely dedicated to the production of gasoline through the MTG process, with a byproduct amount of LPG that is approximately equal to 9 vol % of the total gasoline. The case studies that maximized diesel production were forced to have at least 75 vol % of the liquid product be diesel. All the case studies produced exactly 75 vol % diesel, 25 vol % gasoline, and about 3-5 v/v % byproduct LPG. For the maximum kerosene cases (i.e., at least 75 vol % kerosene), 75 vol % of the products is kerosene, and 25 vol % is an aromatic-rich gasoline blendstock. No byproduct LPG is produced in these cases, as the Cyclar process was used to increase the yield of gasoline and kerosene-range aromatics from the refinery. For these latter sets of case studies, higher volumetric percentages of diesel or kerosene could be obtained through refining of the gasoline fraction, though the resulting GTL refineries would be less economically attractive. The composition of the liquid fuels from the United States ratios case studies was fixed for each refinery to be approximately 67 vol % gasoline, 22 vol % diesel, and 11 vol % kerosene. The total amount of LPG formed as a byproduct for these cases is equal to 2 vol % of the total gasoline/diesel/kerosene produced.

Variations in the amount of sequestered and vented CO₂ can be observed across the 24 case studies. For the unrestricted case studies and the maximum kerosene case studies, the amount of vented CO₂ represents ˜75% of the total CO₂ that is output from the process. The United States ratio studies show a decrease in the vented CO₂ to about 67% of the total, whereas the maximum diesel cases are around 60-65%. It is important to note that the amount of CO₂ sequestration that is utilized is directly a function of the life cycle GHG emissions that are required from the process. If no restriction was placed on the life cycle emissions, then all of the CO₂ that is output from the refinery would simply be vented, resulting in a decrease in the capital and utility costs of the plant. For the near-zero emissions case studies, a significant increase in the amount of sequestered CO₂ is utilized to meet the restriction imposed on these studies.

The electricity production ranges from 1 to 4 MW for 1 kBD plants, 10 to 38 MW for 10 kBD plants, 57 to 218 MW for 50 kBD plants, and 315 to 878 MW for 200 kBD plants. In all cases, the maximum kerosene studies yield the topologies with highest producing electricity, which helps lower the overall fuels cost. The smallest amount of electricity is produced from the near-zero CO₂ venting case studies, which is anticipated due to the higher utility demand for these plants. In general, the electricity output from all the case studies improves the efficiency of the topologies, with the U-10, D-200, and K-10 case studies achieving the highest energy efficiencies (i.e., 75.6, 75.0, and 75.7%, respectively) compared with other case studies in their subcategories (Table 57). The energy efficiency values are calculated by dividing the total energy output (i.e., fuel products, propane, or electricity) by the total energy input (i.e., natural gas or butane). As electricity is output from the system in all case studies, the value is listed as negative in Table 56, and the magnitude of the energy value in Table 57 is added to the total output. If electricity were to be input to the GTL refineries, then this energy value would be added to the total input to the system. The overall energy efficiency of the GTL refineries is above 75.0% for all plant sizes.

TABLE 56
Overall material balance for the 24 case studies

| | Case Study | | | | | | | | | | | |
|---|---|---|---|---|---|---|---|---|---|---|---|---|
| Material Balances | U-1 | U-10 | U-50 | U-200 | D-1 | D-10 | D-50 | D-200 | K-1 | K-10 | K-50 | K-200 |
| Natural gas (mscf/h) | 0.36 | 3.61 | 18.53 | 73.57 | 0.39 | 3.76 | 18.89 | 73.85 | 0.36 | 3.54 | 18.31 | 77.28 |
| Butane (kBD) | — | — | — | — | — | — | — | — | — | — | — | — |
| Water (kBD) | 1.92 | 14.01 | 74.21 | 400.25 | 1.80 | 17.34 | 90.89 | 406.92 | 1.42 | 19.85 | 85.89 | 300.19 |
| Gasoline (kBD) | 1.00 | 10.00 | 50.00 | 200.00 | 0.25 | 2.50 | 12.50 | 50.00 | 0.25 | 2.50 | 12.50 | 50.00 |
| Diesel (kBD) | — | — | — | — | 0.75 | 7.50 | 37.50 | 150.00 | — | — | — | — |
| Kerosene (kBD) | — | — | — | — | — | — | — | — | 0.75 | 7.50 | 37.50 | 150.00 |
| LPG (kBD) | 0.09 | 0.90 | 4.50 | 19.78 | 0.04 | 0.37 | 1.52 | 6.06 | — | — | — | — |
| Seq. CO₂ (tonne/hr) | 1.36 | 10.98 | 84.64 | 347.18 | 2.34 | 16.53 | 91.88 | 265.71 | 1.35 | 10.00 | 73.66 | 587.99 |
| Vented CO₂ (tonne/h) | 4.03 | 40.99 | 204.30 | 796.88 | 3.40 | 35.64 | 176.63 | 706.08 | 4.15 | 40.14 | 214.71 | 808.07 |
| | R-1 | R-10 | R-50 | R-200 | A-U-1 | S-U-1 | P-U-1 | C-U-1 | U-1-Z | U-10-Z | U-50-Z | U-200-Z |
| Natural gas (mscf/h) | 0.36 | 3.62 | 18.02 | 72.20 | 0.39 | 0.36 | 0.39 | 0.37 | 0.36 | 3.61 | 18.53 | 73.57 |
| Butane (kBD) | 0.03 | 0.26 | 1.14 | 4.29 | — | — | — | — | — | — | — | — |
| Water (kBD) | 1.68 | 16.84 | 63.37 | 370.23 | 1.78 | 1.92 | 1.78 | 1.43 | 1.92 | 14.01 | 74.21 | 400.25 |
| Gasoline (kBD) | 0.67 | 6.72 | 33.60 | 134.39 | 1.00 | 1.00 | 1.00 | 1.00 | 1.00 | 10.00 | 50.00 | 200.00 |
| Diesel (kBD) | 0.22 | 2.15 | 10.77 | 43.10 | — | — | — | — | — | — | — | — |
| Kerosene (kBD) | 0.11 | 1.13 | 5.63 | 22.51 | — | — | — | — | — | — | — | — |
| LPG (kBD) | 0.02 | 0.16 | 0.74 | 3.01 | 0.09 | 0.09 | 0.10 | 0.10 | 0.09 | 0.09 | 4.50 | 19.78 |
| Seq. CO₂ (tonne/hr) | 2.02 | 20.25 | 114.88 | 463.60 | 3.07 | 1.36 | 2.76 | 1.88 | 5.33 | 51.46 | 286.05 | 1132.61 |
| Vented CO₂ (tonne/h) | 3.86 | 38.59 | 174.36 | 696.58 | 3.65 | 4.03 | 4.05 | 4.04 | 0.05 | 0.52 | 2.89 | 11.44 |

The inputs to the GTL refinery are natural gas, butane, and water, whereas the outputs include gasoline, diesel, kerosene, LPG, sequestered CO₂, and vented CO₂.

TABLE 57
Overall energy balance for the 24 case studies

| Energy | Case Study | | | | | | | | | | | |
|---|---|---|---|---|---|---|---|---|---|---|---|---|
| Balances (MW) | U-1 | U-10 | U-50 | U-200 | D-1 | D-10 | D-50 | D-200 | K-1 | K-10 | K-50 | K-200 |
| Natural gas | 97 | 958 | 4918 | 19,524 | 102 | 999 | 5013 | 19,599 | 96 | 938 | 4858 | 20,510 |
| Butane | — | — | — | — | — | — | — | — | — | — | — | — |
| Gasoline | 64 | 644 | 3219 | 12,743 | 16 | 159 | 796 | 3186 | 16 | 159 | 796 | 3186 |
| Diesel | — | — | — | — | 53 | 533 | 2667 | 10,668 | — | — | — | — |
| Kerosene | — | — | — | — | — | — | — | — | 52 | 519 | 2596 | 10,384 |
| LPG | 5 | 55 | 273 | 1202 | 2 | 23 | 92 | 368 | — | — | — | — |
| Electricity | −2 | −25 | −141 | −460 | −2 | −25 | −134 | −475 | −4 | −32 | −218 | −878 |
| Efficiency (%) | 74.8 | 75.6 | 73.9 | 73.8 | 72.3 | 74.1 | 73.6 | 75.0 | 74.7 | 75.7 | 74.3 | 70.4 |
| | R-1 | R-10 | R-50 | R-200 | A-U-1 | S-U-1 | P-U-1 | C-U-1 | U-1-Z | U-10-Z | U-50-Z | U-200-Z |
| Natural gas | 96 | 960 | 4782 | 19,159 | 103 | 97 | 102 | 98 | 97 | 958 | 4918 | 19,524 |
| Butane | 2 | 16 | 69 | 260 | — | — | — | — | — | — | — | — |
| Gasoline | 43 | 428 | 2141 | 8563 | 64 | 64 | 64 | 64 | 64 | 644 | 3219 | 12,743 |
| Diesel | 15 | 153 | 766 | 3065 | 0 | 0 | 0 | 0 | — | — | — | — |
| Kerosene | 8 | 78 | 390 | 1558 | 0 | 0 | 0 | 0 | — | — | — | — |
| LPG | 1 | 10 | 45 | 183 | 6 | 5 | 6 | 6 | 5 | 55 | 273 | 1202 |
| Electricity | −4 | −38 | −141 | −563 | −2 | −2 | −3 | −3 | −1 | −10 | −57 | −315 |
| Efficiency (%) | 72.5 | 72.5 | 71.8 | 71.7 | 70.2 | 74.8 | 71.1 | 73.4 | 73.3 | 74.0 | 72.2 | 73.0 |

The energy inputs to the GTL refinery come from natural gas and butane, and the energy outputs are gasoline, diesel, kerosene, LPG, and electricity. The energy efficiency of the process is calculated by dividing the total energy output with the total energy inputs to the process.

### Example 4.14

### Carbon and GHG Balances

The overall carbon balance for the GTL refineries is shown in Table 58 and highlights the eight major points where carbon is either input or output from the system. Carbon that is input to the system via air is neglected due to the low flow rate relative to the other eight points. Over 99% of the input carbon is supplied from the natural gas, whereas the balance is supplied by the butane input to the isomerization and alkylation units. The trends seen in liquid fuel production from Table 56 are consistently displayed in the output carbon flow rates in Table 58. As the percentage of carbon in each of the liquid products is relatively similar, this implies that the relative rates of carbon flow associated with each fuel will be consistent with the volumetric flow rate of each product. The output amount of carbon in the total gasoline, diesel, and kerosene products is, therefore, approximately constant for each plant capacity. The amount of carbon leaving as LPG is around 2-7% of that leaving as gasoline, kerosene, and diesel.

TABLE 58
Carbon balances (in kg/s) for the optimal solutions for the 24 case studies

| | Case Study | | | | | | | | | | | |
|---|---|---|---|---|---|---|---|---|---|---|---|---|
| | U-1 | U-10 | U-50 | U-200 | D-1 | D-10 | D-50 | D-200 | K-1 | K-10 | K-50 | K-200 |
| Natural gas | 1.65 | 16.34 | 83.88 | 333.01 | 1.75 | 17.04 | 85.51 | 334.29 | 1.64 | 16.00 | 82.86 | 349.81 |
| Butane | — | — | — | — | — | — | — | — | — | — | — | — |
| Gasoline | 1.17 | 11.73 | 58.64 | 231.63 | 0.29 | 2.90 | 14.48 | 57.91 | 0.29 | 2.90 | 14.48 | 57.91 |
| Diesel | — | — | — | — | 0.99 | 9.91 | 49.56 | 198.23 | — | — | — | — |
| Kerosene | — | — | — | — | — | — | — | — | 0.93 | 9.30 | 46.52 | 186.08 |
| LPG | 0.07 | 0.67 | 3.33 | 14.66 | 0.03 | 0.28 | 1.12 | 4.49 | — | — | — | — |
| Vented CO₂ | 0.31 | 3.11 | 15.49 | 60.41 | 0.26 | 2.70 | 13.39 | 53.52 | 0.31 | 3.04 | 16.28 | 61.25 |
| Seq. CO₂ | 0.10 | 0.83 | 6.42 | 26.32 | 0.18 | 1.25 | 6.96 | 20.14 | 0.10 | 0.76 | 5.58 | 44.57 |
| % conversion | 75.2 | 75.9 | 73.9 | 74.0 | 75.1 | 76.8 | 76.2 | 78.0 | 74.5 | 76.2 | 73.6 | 69.7 |
| | R-1 | R-10 | R-50 | R-200 | A-U-1 | S-U-1 | P-U-1 | C-U-1 | U-1-Z | U-10-Z | U-50-Z | U-200-Z |
| Natural gas | 1.64 | 16.37 | 81.56 | 326.78 | 1.75 | 1.65 | 1.75 | 1.68 | 1.65 | 16.34 | 83.88 | 333.01 |
| Butane | 0.02 | 0.23 | 1.04 | 3.92 | — | — | — | — | — | — | — | — |
| Gasoline | 0.78 | 7.78 | 38.91 | 155.64 | 1.17 | 1.17 | 1.17 | 1.17 | 1.17 | 11.73 | 58.64 | 231.63 |
| Diesel | 0.28 | 2.85 | 14.24 | 56.95 | — | — | — | — | — | — | — | — |
| Kerosene | 0.14 | 1.40 | 6.98 | 27.93 | — | — | — | — | — | — | — | — |
| LPG | 0.01 | 0.12 | 0.55 | 2.23 | 0.07 | 0.07 | 0.07 | 0.07 | 0.07 | 0.67 | 3.33 | 14.66 |
| Vented CO₂ | 0.29 | 2.93 | 13.22 | 52.80 | 0.28 | 0.31 | 0.31 | 0.31 | 0.00 | 0.04 | 0.22 | 0.87 |
| Seq. CO₂ | 0.15 | 1.53 | 8.71 | 35.14 | 0.23 | 0.10 | 0.21 | 0.14 | 0.40 | 3.90 | 21.68 | 85.86 |
| % conversion | 73.1 | 73.1 | 73.5 | 73.4 | 70.9 | 75.2 | 71.3 | 74.2 | 75.2 | 75.9 | 73.9 | 74.0 |

Carbon is input to the process via natural gas or butanes and exits the process as liquid byproduct, LPG byproduct, vented CO₂, or sequestered (Seq.) CO₂. The small amount of CO₂ input to the system in the purified oxygen stream (<0.01%) is neglected.

For each of the case studies, the carbon conversion rate ranges from 69.7 to 78.0%, with most of the case studies achieving a conversion rate above 70%. The high conversion rates are attributed to two key factors in the GTL refinery, namely the high hydrogen/carbon ratio associated with natural gas and the utilization of CO₂ recycle to increase the overall yield. The first factor is important for the production of a syngas with enough H₂ to convert the CO and CO₂ in the gas with minimal need for CO₂ capture. In fact, the H₂ content associated with steam reforming of natural gas is high enough to allow for input of CO₂ directly into the reformer to help decrease the process CO₂. This second factor is vital for decreasing the capital requirement of all units due to higher carbon yield and for reducing the CO₂ sequestration requirement needed to achieve a proper life cycle GHG target.

The life cycle GHG emission balances for the case studies are shown in Table 59. For each of the studies, the total GHG emission target was set to be at most equal to that for petroleum-based production of liquid fuels or natural gasbased production of electricity. For each liquid product, the amount of GHG produced is calculated by determining the level of CO₂ that would be produced from complete combustion of the product. The life cycle GHG emissions (LGHG) was set to be the sum of the total emissions from each stage of the process. The GHG emissions avoided from liquid fuels (GHGAF) are equivalent to the total energy of fuels produced multiplied by a typical petroleum-based emissions level (i.e., 91.6 kg CO_{2eq}/GJ^{LHV}), whereas the GHG emissions avoided from electricity (GHGAE) are equivalent to the energy produced by electricity multiplied by a typical natural gas-based emissions level (i.e., 101.3 kg CO_{2eq}/GJ). The GHG emissions index (GHGI) represents the division of LGHG by the sum of GHGAF and GHGAE, and values less than unity are indicative processes with superior life cycle GHG emissions than current processes.

The GHG emission rates (in kg CO_{2eq}/s) for the eight major point sources in the refinery are listed in Table 59 and include (a) acquisition and transportation of the natural gas and butane feeds, (b) transportation and use of the gasoline, diesel, kerosene, and LPG, (c) transportation and sequestration of any CO₂, and (d) venting of any process emissions. The GHG emissions for feedstock acquisition and transportation in (a), product transportation in (b), and CO₂ transportation in (c) are calculated from the GREET model for wellto-wheel emissions (Argonne National Laboratory, 2008, which is incorporated herein by reference as if fully set forth) and assuming transportation distances for feedstocks (50 miles), products (100 miles), and CO₂ (50 miles). The GHG emissions from product use in (b) are calculated assuming that each product will be completely combusted to generate CO₂ that is simply vented to the atmosphere.

TABLE 59
GHG balances for the optimal solutions for the 24 case studies

| | Case Study | | | | | | | | | | | |
|---|---|---|---|---|---|---|---|---|---|---|---|---|
| | U-1 | U-10 | U-50 | U-200 | D-1 | D-10 | D-50 | D-200 | K-1 | K-10 | K-50 | K-200 |
| Natural gas | 0.97 | 9.58 | 49.19 | 195.28 | 1.02 | 9.99 | 50.14 | 196.03 | 0.96 | 9.38 | 48.59 | 205.14 |
| Butane | — | — | — | — | — | — | — | — | — | — | — | — |
| Gasoline | 4.30 | 42.98 | 214.89 | 848.89 | 1.06 | 10.61 | 53.05 | 212.20 | 1.06 | 10.61 | 53.05 | 212.20 |
| Diesel | — | — | — | — | 3.63 | 36.32 | 181.60 | 726.39 | — | — | — | — |
| Kerosene | — | — | — | — | — | — | — | — | 3.41 | 34.09 | 170.47 | 681.88 |
| LPG | 0.24 | 2.44 | 12.22 | 53.71 | 0.11 | 1.01 | 4.12 | 16.46 | — | — | — | — |
| Vented CO₂ | 1.12 | 11.39 | 56.75 | 221.35 | 0.95 | 9.90 | 49.06 | 196.13 | 1.15 | 11.15 | 59.64 | 224.47 |
| Seq. CO₂ | 0.02 | 0.15 | 1.18 | 4.82 | 0.03 | 0.23 | 1.28 | 3.69 | 0.02 | 0.14 | 1.02 | 8.17 |
| LGHG | 6.65 | 66.54 | 334.22 | 1323.96 | 6.81 | 68.06 | 339.25 | 1350.90 | 6.60 | 65.38 | 332.77 | 1331.85 |
| GHGAF | 6.40 | 63.98 | 319.92 | 1277.36 | 6.57 | 65.53 | 325.70 | 1302.79 | 6.21 | 62.15 | 310.74 | 1242.96 |
| GHGAE | 0.25 | 2.56 | 14.31 | 46.60 | 0.23 | 2.53 | 13.55 | 48.12 | 0.39 | 3.23 | 22.03 | 88.89 |
| GHGI | 1.00 | 1.00 | 1.00 | 1.00 | 1.00 | 1.00 | 1.00 | 1.00 | 1.00 | 1.00 | 1.00 | 1.00 |
| | R-1 | R-10 | R-50 | R-200 | A-U-1 | S-U-1 | P-U-1 | C-U-1 | U-1-Z | U-10-Z | U-50-Z | U-200-Z |
| Natural gas | 0.96 | 9.60 | 47.83 | 191.63 | 1.03 | 0.97 | 1.02 | 0.99 | 0.97 | 9.58 | 49.19 | 195.28 |
| Butane | 0.00 | 0.03 | 0.13 | 0.47 | — | — | — | — | — | — | — | — |
| Gasoline | 2.85 | 28.52 | 142.59 | 570.35 | 4.24 | 4.30 | 4.24 | 4.24 | 4.30 | 42.98 | 214.89 | 848.79 |
| Diesel | 1.04 | 10.43 | 52.17 | 208.70 | — | — | — | — | — | — | — | — |
| Kerosene | 0.51 | 5.12 | 25.58 | 102.34 | — | — | — | — | — | — | — | — |
| LPG | 0.04 | 0.45 | 2.02 | 8.17 | 0.27 | 0.24 | 0.27 | 0.27 | 0.24 | 2.44 | 12.22 | 53.71 |
| Vented CO₂ | 1.07 | 10.72 | 48.43 | 193.49 | 1.04 | 1.12 | 1.13 | 1.12 | 0.01 | 0.14 | 0.80 | 3.18 |
| Seq. CO₂ | 0.03 | 0.28 | 1.60 | 6.44 | 0.04 | 0.02 | 0.04 | 0.03 | 0.13 | 1.30 | 6.49 | 25.96 |
| LGHG | 6.51 | 65.15 | 320.35 | 1281.59 | 6.62 | 6.65 | 6.70 | 6.65 | 5.65 | 55.20 | 277.22 | 1108.86 |
| GHGAF | 6.13 | 61.31 | 306.11 | 1224.61 | 6.39 | 6.40 | 6.39 | 6.39 | 6.39 | 63.87 | 319.34 | 1277.36 |
| GHGAE | 0.38 | 3.84 | 14.25 | 56.98 | 0.23 | 0.25 | 0.31 | 0.26 | 0.10 | 1.02 | 5.72 | 31.91 |
| GHGI | 1.00 | 1.00 | 1.00 | 1.00 | 1.00 | 1.00 | 1.00 | 1.00 | 0.87 | 0.85 | 0.85 | 0.85 |

The total GHG emissions (in CO₂ equivalents−kg CO₂eq/s) for feedstock acquisition and transportation, product transportation and use, CO₂ sequestration, and process venting are shown for each study. Process feedstocks include natural gas and butane, whereas products include gasoline, diesel, kerosene, and LPG.

For each of the first 20 case studies, the GHGI is exactly equal to 1, implying that each of the GTL refineries has emission levels that are exactly equal to current processes. From Table 59, it is clear that a major component of the life cycle emissions are attributed to the liquid fuels. In fact, ˜70% of the life cycle GHG emissions result from combustion of these fuels in light and heavy duty vehicles. The remaining emissions are mostly attributed to acquisition and transportation of the natural gas and process venting. Natural gas is a particularly GHG intensive feedstock due to the small amount of methane that is leaked to the atmosphere during extraction from the ground. Nevertheless, it is still economical to develop GTL processes that can have appropriate GHG emissions targets. The last four case studies provide an indication on how low the life cycle GHG emissions can be for GTL processes. The studies have GHGI values between 0.85 and 0.87, indicating that the life cycle GHG emissions are 13-15% lower than current fossil-fuel processes. In fact, these values are close to the upper bound of GHG emissions reduction for GTL processes that do not produce a significant amount of byproduct electricity. Coproduction of liquid fuels and electricity at similar energy levels will have lower values for GHGI, as almost all of the carbon used to produce electricity can be captured and sequestered. When producing liquid fuels, it is currently not economical to provide on-board carbon capture for transportation vehicles, so the life cycle GHG emissions reduction will have a theoretical upper limit. Note that the introduction of a biomass feedstock to the refinery would allow the refinery to achieve significantly lower levels of life cycle GHG emissions.

This example has detailed the development of an optimization-based framework for the process synthesis of a thermochemical natural gas to liquids refinery. The framework was used to analyze multiple natural gas conversion technologies, hydrocarbon production technologies, and hydrocarbon upgrading technologies to directly compare the technoeconomic and environmental benefits of each approach. The framework also included a simultaneous heat, power, and water integration to compare the costs of utility generation and wastewater treatment in the overall cost of liquid fuels. The proposed optimization model was tested using 24 distinct case studies that are derived from four combinations of products and four plant capacities with restrictions placed on the natural gas conversion technology and the amount of CO₂ vented. The overall conversion of carbon from feedstock to liquid products was consistently found to be over 70%, and the life cycle GHG emissions was equivalent or less than current fossil-fuel processes. Each case study was globally optimized using a branch-and-bound global optimization algorithm to theoretically guarantee that the cost associated with the optimal design was within 3-6% of the best value possible.

The overall cost of liquid fuels production ranges between $101/bbl and $122/bbl for a 1 kBD plant, $64/bbl and $76/bbl for a 10 kBD plant, $57/bbl and $69/bbl for a 50 kBD plant, and $52/bbl and $64/bbl for a 200 kBD plant. The variation in the cost for each capacity is largely due to the refinery complexity needed to produce a desired quantity of liquid fuels. To minimize the overall costs of fuel production, methanol synthesis and the subsequent MTG route provides the optimal conversion pathway. A significant portion of the produced CO₂ can be recycled back to the reformer, and overall carbon conversion percentages of 70% are readily obtainable. Although this pathway assumes that about 10 vol % of the liquid product from the plant will be LPG, the MTG pathway still remains economically superior if the LPG can be refined to aromatic chemicals using the Cyclar process or co-reformed with the natural gas.

In general, the overall costs with hydrocarbon production through methanol synthesis are lower than those through FT synthesis due to the simplicity of the unconverted synthesis gas recycle loop and decrease in complexity that is required for hydrocarbon upgrading. The unreacted synthesis gas from methanol synthesis may be directly recycled to the methanol synthesis unit without a concern for byproduct species that are generated in the unit. However, the unreacted synthesis gas from FT synthesis will contain C₁-C₄ hydrocarbon species that must be separated out via distillation using refrigeration or recycled back to a reformer to prevent build-up of these species in the recycle gas loop. The benefit associated with FT synthesis is the diversity of products that can be obtained from the process. The range of C₁-C₃₀₊ hydrocarbons allows for a diverse array of fuels, chemicals, lubricants, and waxes that can be readily produced through standard refining practices. The process synthesis framework outlined in this example is of significant benefit, because it marks the first work in the scientific literature that is capable of accessing the technoeconomic and environmental tradeoffs with multiple GTL technologies when given a desired production capacity and composition.

### Example 4.15

### Investment Costs

Table 60 illustrates the investment costs (in 2011 $) for all units that are considered in the GTL refinery.

TABLE 60
CBGTL refinery upgrading unit reference capacities, costs (2011$), and scaling factors

| | | | | | | | |
|---|---|---|---|---|---|---|---|
| Description | Cₒ (MM$) | Sₒ | S_{Max} | Units | Scale Basis | sf | Ref. |

Natural Gas Conversion

| | | | | | | | |
|---|---|---|---|---|---|---|---|
| Auto-thermal reformer | 10.26 | 12.2 | 35.0 | kg/s | Natural gas feed | 0.67 | d |
| Steam-methane reformer | 63.74 | 26.1 | 35.0 | kg/s | Natural gas feed | 0.67 | i |
| Partial oxidation reactor | 650.1 | 118.8 | 75.0 | kg/s | Natural gas feed | 0.67 | f |
| Oxidative coupling reactor | 287.62 | 661.9 | 75.0 | kg/s | Natural gas feed | 0.67 | f |

Synthesis Gas Handling/Clean-up

| | | | | | | | |
|---|---|---|---|---|---|---|---|
| Water-gas-shift unit | $3.75 | 150 | 250 | kg/s | Feed | 0.67 | e |
| Rectisol unit | $32.10 | 2.51 | 8.78 | kmol/s | Feed | 0.63 | g |

Hydrocarbon Production

| | | | | | | | |
|---|---|---|---|---|---|---|---|
| Fischer-Tropsch unit | $12.26 | 23.79 | 60.0 | kg/s | Feed | 0.72 | b, c |
| Hydrocarbon recovery column | $0.65 | 1.82 | 25.20 | kg/s | Feed | 0.70 | d |
| Methanol synthesis | $8.22 | 35.647 | — | kg/s | Feed | 0.65 | e |
| Methanol degasser | $3.82 | 11.169 | — | kg/s | Feed | 0.70 | e |
| Methanol-to-gasoline unit | $5.80 | 10.60 | — | kg/s | Feed | 0.65 | a, e |
| Methanol-to-olefins unit | $3.48 | 10.60 | — | kg/s | Feed | 0.65 | a |

Hydrocarbon Upgrading

| | | | | | | | |
|---|---|---|---|---|---|---|---|
| Distillate hydrotreater | $2.25 | 0.36 | 81.90 | kg/s | Feed | 0.60 | d |
| Kerosene hydrotreater | $2.25 | 0.36 | 81.90 | kg/s | Feed | 0.60 | d |
| Naphtha hydrotreater | $0.68 | 0.26 | 81.90 | kg/s | Feed | 0.65 | d |
| Wax hydrocracker | $8.42 | 1.13 | 72.45 | kg/s | Feed | 0.55 | d |
| Naphtha reformer | $4.70 | 0.43 | 94.50 | kg/s | Feed | 0.60 | d |
| C₅-C₆ isomerizer | $0.86 | 0.15 | 31.50 | kg/s | Feed | 0.62 | d |
| C₄ isomerizer | $9.50 | 6.21 | — | kg/s | Feed | 0.60 | d |
| C₃-C₅ alkylation unit | $52.29 | 12.64 | — | kg/s | Feed | 0.60 | d |
| Saturated gas plant | $7.83 | 4.23 | — | kg/s | Feed | 0.60 | d |
| FT ZSM-5 reactor | $4.93 | 10.60 | — | kg/s | Feed | 0.65 | b, c |
| Olefins-to-gasoline/diesel unit | $3.48 | 10.60 | — | kg/s | Feed | 0.65 | a |
| CO₂ separation unit | $5.39 | 8.54 | — | kg/s | Feed | 0.62 | a |
| Deethanizer | $0.58 | 5.13 | — | kg/s | Feed | 0.68 | a, e |
| Absorber column | $0.91 | 0.96 | — | kg/s | Feed | 0.68 | a, e |
| Stabilizer column | $1.03 | 4.57 | — | kg/s | Feed | 0.68 | a, e |
| Splitter column | $1.01 | 3.96 | — | kg/s | Feed | 0.68 | a, e |
| HF alkylation unit | $8.99 | 0.61 | — | kg/s | Feed | 0.65 | a, e |
| LPG/alkylate splitter | $1.06 | 0.61 | — | kg/s | Feed | 0.68 | a, e |

Hydrogen/Oxygen Production

| | | | | | | | |
|---|---|---|---|---|---|---|---|
| Pressure-swing absorption | $7.96 | 0.29 | — | kmol/s | purge gas | 0.65 | h |
| Air separation unit | $27.6 | 21.3 | 41.7 | kg/s | O₂ | 0.5 | h |
| Air compressor | $6.03 | 10 | 30 | MW | electricity | 0.67 | h |
| Oxygen compressor | $8.07 | 10 | 20 | MW | electricity | 0.67 | h |
| Electrolyzcr | $500 | 1 | — | kW | electricity | 0.9 | h |

Heat and Power Integration

| | | | | | | | |
|---|---|---|---|---|---|---|---|
| Gas Turbine | $81.59 | 266 | 334 | MW | electricity | 0.75 | h |
| Steam Turbine | $66.29 | 136 | 500 | MW | electricity | 0.67 | h |

Wastewater Treatment

| | | | | | | | |
|---|---|---|---|---|---|---|---|
| Sour Stripper | $3.992 | 11.59 | — | kg/s | Feed | 0.53 | i |
| Biological Digestor | $4.752 | 115.74 | — | kg/s | Feed | 0.71 | j |
| Reverse Osmosis | $0.317 | 4.63 | — | kg/s | Feed | 0.85 | j |
| Cooling Tower | $4.055 | 4530.30 | — | kg/s | Feed | 0.78 | i |

a Mobil Research and Development, 1978;
b Mobil Research and Development, 1983;
c Mobil Research and Development, 1985
d Bechtel Corporation, 1998;
e National Renewable Energy Laboratory, 2011;
f Fox et al., 1990
g Kreutz et al., 2008;
h Larson et al., 2009;
i National Energy Technology Laboratory, 2010
j Balmer, 1994

### Example 5

### Global Optimization of a MINLP Process Synthesis Model for Thermochemical Based Conversion of Hybrid Coal, Biomass, and Natural Gas to Liquid Fuels

A global optimization framework is proposed for a thermochemical based process superstructure to produce a novel hybrid energy refinery which will convert carbon-based feedstocks (i.e., coal, biomass, and natural gas) to liquid transportation fuels. The mathematical model for process synthesis includes simultaneous heat, power, and water integration and is formulated as a mixed-integer nonlinear optimization (MINLP) problem with nonconvex functions. The MINLP model is large-scale and includes 15,439 continuous variables, 30 binary variables, 15,406 equality constraints, 230 inequality constraints, and 335 nonconvex terms. The nonconvex terms arise from 274 bilinear terms, 1 quadrilinear term, and 60 concave cost functions. The proposed framework utilizes piecewise linear underestimators for the nonconvex terms to provide tight relaxations when calculating the lower bound. The bilinear terms are relaxed using a partitioning scheme that depends logarithmically on the number of binary variables, while the concave functions are relaxed using a linear partitioning scheme. The framework was tested on twelve case studies featuring three different plant capacities and four different feedstock-carbon conversion percentages and is able to solve each study to within a 3.22-8.56% optimality gap after 100 CPU hours. For 50% feedstock carbon conversion, the proposed global optimization framework shows that the break-even oil prices for liquid fuels production are $61.36/bbl for the small case study, $60.45/bbl for the medium case study, and $55.43/bbl for the large case study, while the corresponding efficiencies are 73.9%, 70.5%, and 70.1%, respectively.

### Example 5.1

### Conceptual Design of Process Superstructure

The CBGTL superstructure is designed to co-feed biomass, coal, and natural gas to produce gasoline, diesel, and kerosene. Synthesis gas (syngas) is generated via gasification from biomass or coal or auto-thermal reaction of natural gas and is converted into hydrocarbon products in the Fischer-Tropsch (FT) reactors which are subsequently upgraded to the final liquid fuels. Co-feeding of biomass and coal uses distinct, parallel biomass and coal gasification trains, followed by subsequent mixing of the individual syngas effluent streams. The gasifiers can either operate with only a solid feedstock input or in tandem with additional vapor phase fuel inputs from elsewhere in the refinery.

The raw syngas is split and either directly sent to a gas cleanup area or to a dedicated reverse water-gas-shift unit to consume CO₂ and generate CO. The dedicated unit is included to facilitate the reverse water-gas-shift reaction at temperatures that are lower than the operating temperatures of the gasifiers, but above the operating temperature of the FT reactors. The gases exiting the reverse water-gasshift unit are then sent to the gas cleanup area. Acid gases including CO₂, H₂S, and NH₃ are removed from the syngas via a Rectisol unit prior to use in the FT reactors. The sulfur-rich gases are directed to a Claus recovery process and the recovered CO₂ may be sequestered or recycled to various units to be reacted with H₂ via the reverse water-gas-shift reaction. The CO₂ may be directed to either the gasifiers, the reverse water-gas-shift reactor, or the iron-based FT units. Recovered CO₂ is not sent to the cobalt-based FT units to ensure a maximum molar concentration of 3% within the unit and prevent poisoning of the catalyst. Two FT reactors operate at high temperature (320° C.) and low temperatures (240° C.) and will each be associated with distinct alpha (chain growth probability measure) values.

Fuel quality products are obtained by treating the FT effluent in a detailed upgrading section. Waxes are converted into naphtha and distillate in a hydrocracker unit while hydrotreater units are employed to upgrade the naphtha, distillate, or kerosene. The naphtha cut is further reformed and isomerized to improve the octane number. Lighter forms of hydrocarbons are passed through a series of alkylation and isomerization processes to form high-octane gasoline blending stock. A stream of input butanes is directed to the C4 isomerizer to enhance the quality of the output product. Offgas streams from various upgrading units are combined in a saturated gas plant to recover C4 gases for isomerization or C3 species to be sold as byproduct propane (liquefied petroleum gas). The remaining gases from the saturated gas plant are split to either (i) an auto thermal reactor, (ii) a combustion unit, (iii) a gas turbine engine, or (iv) a pressure-swing adsorption unit.

Hydrogen is produced via pressure-swing adsorption or an electrolyzer unit while oxygen can be provided by the electrolyzer or a separate cryogenic air separation unit. Heat and power integration is incorporated into the process superstructure using a series of heat engines and the approach of Duran and Grossmann, 1986, which is incorporated herein by reference as if fully set forth. Steam for the process units is also provided by boiling condensate using waste-heat from the process. To accompany the above process superstructure, a complete water treatment network is postulated that will treat and recycle (a) wastewater from various process units, (b) blowdown from the cooling tower, (c) blowdown from the boilers, and (d) input freshwater. The graphical representation of this superstructure is included as Supplementary Information.

### Example 5.2

### Mathematical Model Nonlinearities

This section will focus on the nonlinearities that are present within the mathematical model for process synthesis with simultaneous heat, power, and water integration. Specifically, each portion of the CBGTL process topology that gives rise to a nonlinear series of equations will be discussed along with the number of nonlinear terms introduced and the anticipated bounds of the variables present in these terms.

### Example 5.2.1

### Origin of Bilinear Terms

The nonconvex bilinear terms within the mathematical model arise from the multiplication of two positive, continuous variables. These terms are found when a stream composition must be specified, a stream with unknown composition must be split, or a detailed chemical equilibrium must be enforced. To reduce the amount of composition variables recorded throughout the process superstructure, the operation of the process units is generally defined using total stream flow rates and the corresponding species flow rates. Material balances can therefore be maintained throughout the process without specifically tracking the stream compositions for each unit inlet and outlet. However, proper operation of some process units will require explicit knowledge of the stream compositions to be determined.

### Example 5.2.1.1

### Flash Units—Phase Equilibrium

Vapor-liquid phase equilibrium within a unit is generally modeled using the formula y=Kx where y is the composition of the vapor phase, x is the composition of the liquid phase, and K is the equilibrium constant. This equilibrium must be maintained within the four flash units (U_{Fl}) of the CBGTL process superstructure (see Table 61). Given a particular flash unit u, the concentration x^{S} of each species s in the liquid phase (u, uL, s) and the vapor phase (u, uv, s) is constrained using Eq. (424), where K^{VLE} is the equilibrium constant.

x_{u,u}_{_{V}}_{,s}^{S}−K_{u,s}^{VLE}·x_{u,u}_{_{L}}_{,s}^{S}=0∀u∈U_{Fl} (424)

The equilibrium constant is generally a function of the temperature, pressure, and composition of the input stream to the unit. In the CBGTL process superstructure, the temperature and pressure of the flash units are fixed. A generic input composition is used to derive the value of the equilibrium constants from Aspen Plus using the Peng-Robinson equation of state with the Boston-Mathias alpha function. It is then assumed that the values of the equilibrium constant will be independent of the variations in the species concentration seen in the input stream, so the Aspen Plus values will be constants in the mathematical model. The stream compositions entering the flash units in the optimization model will not vary significantly (±2%) from the generic composition used in the Aspen Plus simulation, so the assumption is justified. If the stream compositions were to have large ranges in the optimization model, then the equilibrium constant may need to be represented as a variable function of the composition entering the flash unit.

To establish the species concentrations in the liquid and vapor phases, Eqs. (425) and (426) are used along with the species (NS) and total (NT) molar flow rates. Note that the bilinear terms arise from the combination of total molar flow rate and species concentration in Eqs. (425) and (426). Each equation contains |S| bilinear terms where |S| is the total number of species in the flash unit. There are a total of four flash units within the process, each of which may contains a different number of species. Table 61 details that the acid gas flash unit (AGF) has 38 bilinear terms arising from 19 species, the Claus flash unit (CF) has 26 bilinear terms from the 13 species present, and the fuel combustor flash (FCF) and gas turbine flash (GTF) units each have 14 bilinear terms due to the 7 possible species that can be present.

x_{u,u}_{_{L}}_{,s}^{S}·N_{u,u}_{_{L}}^{T}−N_{u,u}_{_{L}}_{,s}^{S}=0∀u∈U_{Fl} (425)

x_{u,u}_{_{V}}_{,s}^{S}·N_{u,u}_{_{V}}^{T}−N_{u,u}_{_{V}}_{,s}^{S}=0∀u∈U_{Fl} (426)

All terms in Eqs. (425) and (426) are of the form (x^{S}·N^{T}) which multiplies a tightly bound species concentration variable by a total flow rate variable. The composition variables begin with a range of [0,1] which can be reduced according to the restrictions of Eqs. (427) and (428) for the liquid and vapor phases, respectively. These restrictions are based on the vapor-liquid equilibrium equation shown in Eq. (429). For the liquid phase, the maximum concentration can be established by dividing the maximum concentration of the vapor phase by the equilibrium constant. For the vapor phase, the maximum concentration can be established by multiplying the maximum concentration of the liquid phase by the equilibrium constant. Both the liquid and vapor phase concentration cannot be greater than 1, so Eqs. (427) and (428) ensure this as well. Restrictions on the variable bounds will aid in providing a tighter relaxation during the global optimization routine.

$$\begin{matrix}{χ}_{u , {u}_{L} , s}^{S} - min \{ 1 , \frac{1}{{K}_{u , s}^{VLE}} \} \leq 0 \\ ∀ ( u , {u}_{L} , s ) ∈ {S}^{UF} , u ∈ {U}_{FI} & ( 427 ) \\ {χ}_{u , {u}_{V} , s}^{S} - min \{ 1 , {K}_{u , s}^{VLE} \} \leq 0 \\ ∀ ( u , {u}_{V} , s ) ∈ {S}^{UF} , u ∈ {U}_{FI} & ( 428 ) \\ {χ}_{u , {u}_{V} , s}^{S} - {K}_{u , s}^{VLE} \cdot {χ}_{u , {u}_{L} , s}^{S} = 0 \\ ∀ ( u , {u}_{L} , s ) ∈ {S}^{UF} , u ∈ {U}_{FI} & ( 429 )\end{matrix}$$

TABLE 61
Information pertaining to the origin of bilinear terms in the mathematical model.

| | | No. of | No. of |
|---|---|---|---|
| | | outlet | bilinear |
| Unit description | Inlet species | streams | terms |
| Flash units - phase equilibrium | | | |
| Acid gas flash (AGF) | Ar, CH₄, CO, CO₂, C₂H₂, C₂H₄ | 2 | 38 |
| | C₂H₆, H₂, H₂O, NO, N₂O, HCN | | |
| | H₂S, SO₂, HCl, COS, NH₃, N₂, O₂ | | |
| Claus flash (CF) | Ar, CO₂, H₂O, NO, N₂O, HCN | 2 | 26 |
| | H₂S, SO₂, HCl, COS, NH₃, N₂, O₂ | | |
| Fuel combustor flash (FCF) | Ar, CO₂, H₂O, NO, N₂O, N₂, O₂ | 2 | 14 |
| Gas turbine flash (GTF) | Ar, CO₂, H₂O, NO, N₂O, N₂, O₂ | 2 | 14 |
| | | Total: | 92 |
| Splitter units - stream splitting | | | |
| Tar cracker splitter (SP_{TCK}) | Ar, CH₄, CO, CO₂, C₂H₂, C₂H₄ | 2 | 19 |
| | C₂H₈, H₂, H₂O, NO, N₂O, HCN | | |
| | H₂S, SO₂, HCl, COS, NH₃, N₂, O₂ | | |
| Coal cyclone splitter (SP_{CC2}) | Ar, CH₄, CO, CO₂, C₂H₂, C₂H₄ | 2 | 19 |
| | C₂/H₆, H₂, H₂O, NO, N₂O, HCN | | |
| | H₂S, SO₂, HCl, COS, NH₃, N₂, O₂ | | |
| Clean gas splitter (SP_{AGR}) | Ar, CH₄, CO, CO₂, C₂H₂, C₂H₄ | 2 | 13 |
| | C₂H₆, H₂, H₂O, NO, N₂O, N₂, O₂ | | |
| Fischer-Tropsch splitter (SP_{FPC}) | Ar, CH₄, CO, CO₂, C₂H₂, C₂H₄ | 2 | 13 |
| | C₂H₆, H₂, H₂O, NO, N₂O, N₂, O₂ | | |
| Acid gas splitter (SP_{AGC}) | CO₂, HCN, H₂S, SO₂, COS, NH₃ | 2 | 6 |
| Kerosene splitter (SP_{KER}) | C₁₁H₂₂, C₁₁H₂₄, C₁₂H₂₄ | 2 | 6 |
| | C₁₂H₂₆, C₁₃H₂₆, C₁₃C₂₈ | | |
| Gas turbine effluent splitter (SP_{CT}) | Ar, CO₂, H₂O, NO, N₂O, N₂, O₂ | 2 | 7 |
| Auto-thermal reactor splitter (SP_{ATR}) | Ar, CH₄, CO, CO₂, C₂H₂, C₂H₄ | 5 | 52 |
| | C₂H₆, H₂, H₂O, NO, N₂O, N₂, O₂ | | |
| Sour stripper bottoms splitter (WRN_{SSS}) | H₂O, NH₃ | 2 | 2 |
| Reverse osmosis splitter (WRN_{SRO}) | H₂O, TDS | 4 | 6 |
| Deaerator effluent splitter (WRN_{SDA}) | H₂O, TDS | 2 | 2 |
| Fischer-Tropsch wastewater splitter (WRN_{SFT}) | H₂O, CO₂, OXVAP, OXH₂O, OXHC | 2 | 5 |
| Post-combustion wastewater splitter (WRN_{SPC}) | H₂O, CO₂, NO, N₂O, Ar, O₂, N₂ | 3 | 14 |
| | | Total: | 164 |
| Reactors - chemical equilibrium | | | |
| Biomass gasifier (BCI) | (CO, H₂O), (CO₂, H₂) | 1 | 2 |
| Coal gasfier (X_{CGS}) | (CO, H₂O), (CO₂, H₂) | 1 | 2 |
| Reverse water-gas-shift unit (X_{RGS}) | (CO, H₂O), (CO₂, H₂) | 1 | 2 |
| High-temp. iron-based Fischer-Tropsch (HTFTRGS) | (CO, H₂O), (CO₂, H₂) | 1 | 2 |
| Low-temp. iron-based Fischer-Tropsch (LTFTRGS) | (CO, H₂O), (CO₂, H₂) | 1 | 2 |
| Auto-thermal reactor (ATR) - water-gas shift | (CO, H₂O), (CO₂, H₂) | 1 | 2 |
| Auto-thermal reactor (ATR) - CH₄ reforming | (CH₄, H₂O), (CO₂, H₂) | 1 | 2 |
| Auto-thermal reactor (ATR) - C₂H₂ reforming | (CH₄, CO), (C₂H₂, H₂O) | 1 | 2 |
| Auto-thermal reactor (ATR) - C₂H₄ reforming | (C₂H₂, H₂) | 1 | 1 |
| Auto-thermal reactor (ATR) - C₂H₆ reforming | (C₂H₄, H₂) | 1 | 1 |
| | | Total: | 18 |
| | Total bilinear terms: | | 274 |

The name in parenthesis represents the unit in the CBGTL superstructure (Supp. information) for which the nonlinear equations are enforced on the outlet streams. These terms arise due to vapor-liquid phase equilibrium within the flash units, chemical equilibrium within specific reactor units, and stream splitting at the splitter units. The number of bilinear terms for each of the flash units is equal to the number of species times the number of outlet streams. For the splitter units, the number of bilinear terms is equal to the number of species times one less than the number of outlet streams. Two bilinear terms are needed for each reactor constrained by the water-gas-shift reaction, and six additional bilinear terms are needed within the auto-thermal reactor to govern equilibrium of steam reforming reactions for the hydrocarbons.

Note that Eq. (424) could be reformulated as Eq. (430) without introducing the species concentration variables:

N_{u,u}_{_{L}}^{T}·N_{u,u}_{_{V}}_{,s}^{S}−K_{u,s}^{VLE}·N_{u,u}_{_{V}}^{T}·N_{u,u}_{_{L}}_{,s}^{S}=0∀u∈U_{Fl} (430)

This would introduce an equivalent number of bilinear terms, though each of the bilinear terms would be of the form (N^{S}·N^{T}). The increased range of the N^{S} variables would lead into relaxations that are looser than those provided with the species concentration variables. Therefore, this study focused on the bilinear terms developed using Eqs. (425) and (426).

### Example 5.2.1.2

### Splitter Units Stream Splitting

Proper operation of all splitter units (U_{Sp}) requires the composition of all outlet streams, (u, u′), to be equal to that of the inlet stream, (uₗ, u). This may be done by defining stream concentration variables, xᵤ_{_{I}}_{,u,s}^{S} for each species in the inlet stream and constraining all outlet streams to have this exact concentration. Without loss of generality, the species flow rates for each exiting stream can then be set using the composition of the entering stream (Eq. (431)).

N_{u,u′,s}^{S}−xᵤ_{_{I}}_{,u,s}^{S}·N_{u,u′}^{T}=0∀(u,u′,s)∈S^{UF},u∈U_{Sp} (431)

Note that a species balance around the splitter unit will prevent the need for Eq. (431) on the splitter inlet. Eq. (431) introduces a total of (|S|·|U|) bilinear terms for each splitter unit, where |S| is the total number of species entering the splitter unit and |U| is the total number of output streams.

An alternative formulation of the stream splitters is to use split fractions, sp_{u,u′}, for each outlet stream. In such an approach, the outlet stream species flow rates will be governed using Eq. (432) by multiplying the split fraction by the inlet species flow rate. Eq. (433) enforces that all of the split fractions will sum to one. Note that Eq. (432) does not have to be utilized for one of the outlet streams from the splitter due to the species material balance around the unit and will therefore require (|S|·{|U|−1}) bilinear terms. In this formulation, the number of bilinear terms is reduced by |S| for each splitter unit as opposed to the previous formulation. Note that the species flow rates in the current formulation and the total molar flow rates in the previous formulation can be scaled to be in the continuous range of [0, 1]. Therefore, all of the variables that participate in either formulation would be in the continuous range of [0, 1], which generally results in increased computational performance.

$$\begin{matrix}{N}_{u , {u}^{′} , s} - {sp}_{u , {u}^{′}} \cdot {N}_{{u}_{I} , u , s}^{S} = 0 \\ ∀ ( u , {u}^{′} , s ) ∈ {S}^{UF} , u ∈ {U}_{Sp} & ( 432 ) \\ {\sum }_{( u , {u}^{′} ) ∈ UC} {sp}_{u , {u}^{′}} - 1 = 0 \\ ∀ u ∈ {U}_{Sp} & ( 433 )\end{matrix}$$

While both sets of equations are equally valid representations of the splitter units, each formulation will affect the complexity and solution quality of the linear relaxation of the mathematical model differently. The splitter bilinear terms are modeled using piecewise linear underestimators which require binary variables to partition the range of a particular variable in the bilinear term. It is important to consider the role of piecewise linear underestimation of the bilinear terms using binary variables. For Eq. (431), either the N_{u,u′}^{T} or the xᵤ_{_{I}}_{,u,s}^{S} variables are candidates for the piecewise linear relaxation. Using the N_{u,u′}^{T} variables will introduce |U|·|P| binary variables where |P| is the total number of binary variables introduced to define the activation of a specific partition of one term. If the xᵤ_{_{I}}_{,u,s}^{S} variables are used, then |S|·|P| binary variables are required. Due to the large amount of species present in each splitter unit, the introduction of binary variables for the N_{u,u′}^{T} variables is more computationally efficient.

For Eq. (432), the same reasoning leads to the selection of the sp_{u,u′} variables for range partitioning using the binary variables. Note that for this latter formulation, the number of binary variables introduced will be less than the former formulation by |P| for each splitter unit. Additionally, the stream flow rate variables (Nᵤ_{_{I}}_{,u,s}^{S}) will have a lower bound than the total flow rate variables (N_{u,u′}^{T}). These two factors combine to make the latter formulation a more attractive choice for the piecewise-linear underestimation of the bilinear terms. This study will focus on the bilinear terms introduced in Eq. (432) with the intention of using binary variables to partition the range of the spu,u′ variables.

### Example 5.2.1.3

### Reactor Units—Chemical Equilibrium

A majority of the units in the process superstructure requiring chemical equilibrium are solely based on the water-gas-shift reaction. That is, the species flow rates in a given stream, N_{u,u′,s}^{S}, are constrained via the general equation shown in Eq. (434).

N_{u,u′,CO}^{S}·N_{u,u′,H}_{₂}_{O}^{S}−Kᵤ^{WGS}·N_{u,u′,CO}_{₂}^{S}·N_{u,u′,H}_{₂}^{S}=0∀(u,u′)∈U_{WGS} (434)

U_{WGS} is defined as the set of all streams for which the water-gas-shift equation must be enforced. If the unit operating temperature of the unit is unknown, then the chemical equilibrium coefficient, Kᵤ^{WGS}, must be a variable, with a value chosen based on the operating temperature selected by the optimization model. This would require the use of trilinear (or higher order) terms to define this equation since the variable equilibrium constant must also be included in the equation. Additionally, the mathematical equation defining the value of the equilibrium constant may be a nonlinear exponential function if the temperature range is continuous. If the temperature of the unit is selected from a discrete set of values, then the mathematical definition of the equilibrium constant will be a linear function of the binary variables for the temperature choices and the parametric values for the equilibrium constant at each temperature. Linear relaxation of the trilinear terms can be properly incorporated by using underestimators to model the convex hull surrounding the term (Meyer & Floudas, 2003, 2004) or by combining two of the variables to form an auxiliary bilinear term and then combining the auxiliary term with the third variable to form a second bilinear term. These two bilinear terms can then be relaxed using piecewise linear underestimators as defined previously. An additional consequence of the use of a continuous temperature range is the addition of non-linear constraints to define the heat and power integration (Duran & Grossmann, 1986). This enhanced computational complexity is not necessary if the operating temperature of the unit may be chosen from a finite set of discrete values (Baliban et al., 2011).

Selection of one of the temperature values is logically enforced using a binary variable, yᵤ, which will simultaneously select the temperature value and the equilibrium coefficient for the reactor unit. Note that this formulation allows Eq. (434) to be rewritten as Eqs. (435) and (436).

N_{u,u′,CO}^{S}·N_{u,u′,H}_{₂}_{O}^{S}−K_{u,u′,u}_{_{WGS}}^{WGS}·N_{u,u′,CO}_{₂}^{S}·N_{u,u′,H}_{₂}^{S}−N_{u,u′,CO}^{S-UB}·N_{u,u′,H}_{₂}_{O}^{S-UB}·(1−yᵤ_{_{WGS}})≦0∀(u,u′,u_{WGS})∈U_{WGS} (435)

K_{u,u′,u}_{_{WGS}}^{WGS}·N_{u,u′,CO}_{₂}^{S}·N_{u,u′,H}_{₂}^{S}−N_{u,u′,CO}^{S}·N_{u,u′,CO}^{S}·N_{u,u′,H}_{₂}_{O}^{S}−K_{u,u′,u}_{_{WGS}}^{WGS}·N_{u,u′,CO}_{₂}^{S-UB}·N_{u,u′,H}_{₂}^{S-UB}·(1−yᵤ_{_{WGS}})≦0∀(u,u′,u_{_{WGS}})∈U_{WGS} (436)

U_{WGS} has been defined here to mean the set of all streams (u, u′) for which the water-gas-shift equilibrium must be enforced using the operating conditions of unit u_{WGS}. The value N_{u,u′,s}^{S-UB} represents the upper bound on the flow rate for stream (u, u′, s). There are a total of six units including the gasifiers, the auto-thermal reactor, the reverse water-gas-shift reactor, and the iron-based FT units that must enforce the water-gas-shift equilibrium. Each unit will require two bilinear terms in the model, leading to 12 total bilinear terms.

The auto-thermal reactor must also enforce steam reforming equilibrium for the four output hydrocarbon species (CH₄, C₂H₂, C₂H₄, and C₂H₆). The general form for the steam reforming reactions is shown in Eqs. (437)-(440).

N_{u,u′,CO}^{S}·N_{u,u′,H}_{₂}^{S}^{³}−K_{u,CH}_{₄}^{SR}·N_{u,u′,CH}_{₄}^{S}·N_{u,u′,H}_{₂}_{O}^{S}=0∀(u,u′)∈UC,u∈U_{ATR} (437)

N_{u,u′,CO}^{S}^{²}·N_{u,u′,H}_{₂}^{S}^{³}−K_{u,C}_{₂}_{H}_{₂}^{SR}·N_{u,u′,C}_{₂}_{H}_{₂}^{S}·N_{u,u′,H}_{₂}_{O}^{S}^{²}=0∀(u,u′)∈UC,u∈U_{ATR} (438)

N_{u,u′,CO}^{S}^{²}·N_{u,u′,H}_{₂}^{S}^{⁴}−K_{u,C}_{₂}_{H}_{₄}^{SR}·N_{u,u′,C}_{₂}_{H}_{₄}^{S}·N_{u,u′,H}_{₂}_{O}^{S}^{²}=0∀(u,u′)∈UC,u∈U_{ATR} (439)

N_{u,u′,CO}^{S}·N_{u,u′,H}_{₂}^{S}^{⁵}−K_{u,C}_{₂}_{H}_{₆}^{SR}·N_{u,u′,C}_{₂}_{H}_{₆}^{S}·N_{u,u′,H}_{₂}_{O}^{S}^{²}=0∀(u,u′)∈UC,u∈U_{ATR} (440)

Note that combining Eqs. (437) and (438) can produce Eq. (441). Eq. (442) can be produced from Eqs. (438) and (439) and Eq. (443) can be produced from Eqs. (439) and (440).

$$\begin{matrix}{N}_{u , {u}^{′} , {C}_{2} {H}_{2}}^{S} \cdot {N}_{u , {u}^{′} , {H}_{2} O}^{S} - \frac{{K}_{u , {CH}_{4}}^{SR}}{{K}_{u , {C}_{2} {H}_{2}}^{SR}} \cdot {N}_{u , {u}^{′} , {CH}_{4}}^{S} \cdot {N}_{u , {u}^{′} , CO}^{S} = 0 \\ ∀ ( u , {u}^{′} ) ∈ UC , u ∈ {U}_{ATR} & ( 441 ) \\ {N}_{u , {u}^{′} , {C}_{2} {H}_{4}}^{S} - \frac{{K}_{u , {C}_{2} {H}_{2}}^{SR}}{{K}_{u , {C}_{2} {H}_{4}}^{SR}} \cdot {N}_{u , {u}^{′} , {C}_{2} {H}_{2}}^{S} \cdot {N}_{u , {u}^{′} , {H}_{2}}^{S} = 0 \\ ∀ ( u , {u}^{′} ) ∈ UC , u ∈ {U}_{ATR} & ( 442 ) \\ {N}_{u , {u}^{′} , {C}_{2} {H}_{6}}^{S} - \frac{{K}_{u , {C}_{2} {H}_{4}}^{SR}}{{K}_{u , {C}_{2} {H}_{6}}^{SR}} \cdot {N}_{u , {u}^{′} , {C}_{2} {H}_{4}}^{S} \cdot {N}_{u , {u}^{′} , {H}_{2}}^{S} = 0 \\ ∀ ( u , {u}^{′} ) ∈ UC , u ∈ {U}_{ATR} & ( 443 )\end{matrix}$$

The equilibrium coefficients in Eqs. (437) and (441)-(443) are dependent on the selection of operating temperature within the autothermal reactor. These variables may be eliminated by changing the equality to two inequalities as shown below. Eqs. (444) and (445) are used in place of Eq. (437), Eqs. (446) and (447) in place of Eq. (441), Eqs. (448) and (449) in place of Eq. (442), and Eqs. (450) and (451) in place of Eq. (443).

$$\begin{matrix}{N}_{u , {u}^{′} , CO}^{S} \cdot {N}_{u , {u}^{′} , {H}_{2}}^{S 3} - {K}_{u , {CH}_{4}}^{SR} \cdot {N}_{u , {u}^{′} , {CH}_{4}}^{S} \cdot {N}_{u , {u}^{′} , {H}_{2} O}^{S} - {N}_{u , {u}^{′} , CO}^{S - UB} \cdot {N}_{u , {u}^{′} , {H}_{2}}^{S - UB 3} \cdot ( 1 - {y}_{{u}_{ATR}} ) \leq 0 ∀ ( u , {u}^{′} , {u}_{ATR} ) ∈ {U}_{ATR} & ( 444 ) \\ {K}_{u , {CH}_{4}}^{SR} \cdot {N}_{u , {u}^{′} , {CH}_{4}} \cdot {N}_{u , {u}^{′} , {H}_{2} O}^{S} - {N}_{u , {u}^{′} , CO}^{S} \cdot {N}_{u , {u}^{′} , {H}_{2}}^{S 3} - {K}_{u , {CH}_{4}}^{SR} \cdot {N}_{u , {u}^{′} , {CH}_{4}}^{S - UB} \cdot {N}_{u , {u}^{′} , {H}_{2} O}^{S - UB} \cdot ( 1 - {y}_{{u}_{ATR}} ) \leq 0 ∀ ( u , {u}^{′} , {u}_{ATR} ) ∈ {U}_{ATR} & ( 445 ) \\ {N}_{u , {u}^{′} , {C}_{2} {H}_{2}}^{S} \cdot {N}_{u , {u}^{′} , {H}_{2} O}^{S} - \frac{{K}_{u , {CH}_{4}}^{SR}}{{K}_{u , {C}_{2} {H}_{2}}^{SR}} \cdot {N}_{u , {u}^{′} , {CH}_{4}}^{S} \cdot {N}_{u , {u}^{′} , CO}^{S} - {N}_{u , {u}^{′} , {C}_{2} {H}_{2}}^{S - UB} \cdot {N}_{u , {u}^{′} , {H}_{2} O}^{S - UB} \cdot ( 1 - {y}_{{u}_{ATR}} ) \leq 0 ∀ ( u , {u}^{′} , {u}_{ATR} ) ∈ {U}_{ATR} & ( 446 ) \\ \frac{{K}_{u , {CH}_{4}}^{SR}}{{K}_{u , {C}_{2} {H}_{2}}^{SR}} \cdot {N}_{u , {u}^{′} , {CH}_{4}}^{SR} \cdot {N}_{u , {u}^{′} , CO}^{S} - {N}_{u , {u}^{′} , {C}_{2} {H}_{2}}^{S} \cdot {N}_{u , {u}^{′} , {H}_{2} O}^{S} - \frac{{K}_{u , {CH}_{4}}^{SR}}{{K}_{u , {C}_{2} {H}_{2}}^{SR}} {N}_{u , {u}^{′} , {CH}_{4}}^{S - UB} \cdot {N}_{u , {u}^{′} , CO}^{S - UB} \cdot ( 1 - {y}_{{u}_{ATR}} ) \leq 0 ∀ ( u , {u}^{′} , {u}_{ATR} ) ∈ {U}_{ATR} & ( 447 ) \\ {N}_{u , {u}^{′} , {C}_{2} {H}_{4}}^{S} - \frac{{K}_{u , {CH}_{4}}^{SR}}{{K}_{u , {C}_{2} {H}_{4}}^{SR}} \cdot {N}_{u , {u}^{′} , {C}_{2} {H}_{2}}^{S} \cdot {N}_{u , {u}^{′} , {H}_{2}}^{S} - {N}_{u , {u}^{′} , {C}_{2} {H}_{4}}^{S - UB} \cdot ( 1 - {y}_{{u}_{ATR}} ) \leq 0 ∀ ( u , {u}^{′} , {u}_{ATR} ) ∈ {U}_{ATR} & ( 448 ) \\ \frac{{K}_{u , {C}_{2} {H}_{2}}^{SR}}{{K}_{u , {C}_{2} {H}_{4}}^{SR}} \cdot {N}_{u , {u}^{′} , {C}_{2} {H}_{2}}^{S} \cdot {N}_{u , {u}^{′} , {H}_{2}}^{S} - {N}_{u , {u}^{′} , {C}_{2} {H}_{4}}^{S} - \frac{{K}_{u , {C}_{2} {H}_{2}}^{SR}}{{K}_{u , {C}_{2} {H}_{4}}^{SR}} \cdot {N}_{u , {u}^{′} , {C}_{2} {H}_{2}}^{S - UB} \cdot {N}_{u , {u}^{′} , {H}_{2}}^{S - UB} \cdot ( 1 - {y}_{{u}_{ATR}} ) \leq 0 ∀ ( u , {u}^{′} , {u}_{ATR} ) ∈ {U}_{ATR} & ( 449 ) \\ {N}_{u , {u}^{′} , {C}_{2} {H}_{6}}^{S} - \frac{{K}_{u , {C}_{2} {H}_{4}}^{SR}}{{K}_{u , {C}_{2} {H}_{6}}^{SR}} \cdot {N}_{u , {u}^{′} , {C}_{2} {H}_{4}}^{S} \cdot {N}_{u , {u}^{′} , {H}_{2}}^{S} - {N}_{u , {u}^{′} , {C}_{2} {H}_{6}}^{S} \cdot ( 1 - {y}_{{u}_{ATR}} ) \leq 0 ∀ ( u , {u}^{′} , {u}_{ATR} ) ∈ {U}_{ATR} & ( 450 ) \\ \frac{{K}_{u , {C}_{2} {H}_{4}}^{SR}}{{K}_{u , {C}_{2} {H}_{6}}^{SR}} \cdot {N}_{u , {u}^{′} , {C}_{2} {H}_{4}}^{S} \cdot {N}_{u , {u}^{′} , {H}_{2}}^{S} - {N}_{u , {u}^{′} , {C}_{2} {H}_{6}}^{S} - \frac{{K}_{u , {C}_{2} {H}_{4}}^{SR}}{{K}_{u , {C}_{2} {H}_{6}}^{SR}} \cdot {N}_{u , {u}^{′} , {C}_{2} {H}_{4}}^{S - UB} \cdot {N}_{u , {u}^{′} , {H}_{2}}^{S - UB} \cdot ( 1 - {y}_{{u}_{ATR}} ) \leq 0 ∀ ( u , {u}^{′} , {u}_{ATR} ) ∈ {U}_{ATR} & ( 451 )\end{matrix}$$

U_{ATR} has been defined to mean the set of all streams (u, u′) which must be enforced using the operating conditions of unit u_{ATR}. Eqs. (444)-(451) are utilized in the mathematical model and introduce five bilinear terms and one quadrilinear term. This quadrilinear term may be underestimated using a variety of convex relaxation techniques (Cafieri, Lee, & Liberti, 2010, which is incorporated herein by reference as if fully set forth), including a bilinear term relaxation and a successive trilinear term relaxation (Meyer & Floudas, 2003, 2004, which are incorporated herein by reference as if fully set forth) or three successive auxiliary bilinear terms.

### Example 5.2.2

### Concave Cost Functions

The investment cost of the final process topology will be calculated as the sum of the investment cost of all representative process units, UInv, throughout the superstructure. Though some units in the superstructure will have a cost function that is solely based on the construction of that unit (e.g., compressors, turbines, and flash units), several of the units will have a cost function that accounts for construction of that unit along with axillary units necessary for proper operation. For example, the investment cost of the biomass gasifier will include the cost of the gasifier and the feed lockhopper. A total of 60 cost curves are needed for the process superstructure, each of which is of the form in Eq. (452).

$${Inv}_{u} = {C}_{u}^{o} \cdot \frac{{S}_{u}^{{sf}_{u}}}{{S}_{u}^{o}} \\ ∀ u ∈ {U}_{Inv} \qquad ( 452 )$$

In this function, Cᵤ^{o} represents the base cost, Sᵤ^{o} represents the base flow rate, Sᵤ represents the working flow rate, and sfᵤ represents the scaling factor. Note that Eq. (452) assumes that units operate without a maximum flow rate. This assumption was utilized to avoid the mathematical complexity associated with the restriction of a maximum flow. If a maximum flow rate, Sᵤ^{M}, is imposed for a unit operation, then the total number of unit trains, nu, and the working flow rate of each train, Stu, must be enforced using Eqs. (454) and (455). The investment cost of each unit would then be calculated using Eq. (455).

$$\begin{matrix}{S}_{u}^{t} \leq {S}_{u}^{M} & ( 453 ) \\ {S}_{u} = {S}_{u}^{t} \cdot {n}_{u} & ( 454 ) \\ {Inv}_{u} = {n}_{u}^{0.9} \cdot {C}_{u}^{o} \cdot \frac{{S}_{u}^{{sf}_{u}}}{{n}_{u} \cdot {S}_{u}^{o}} \\ ∀ u ∈ {U}_{Inv} & ( 455 )\end{matrix}$$

Note that Eq. (455) will contain a discontinuity at all points where the working flow is an integer multiple of the maximum flow. Additionally, binary variables would be required to logically define the number of units necessary to operate given the restrictions on the unit capacity. To circumvent this computational burden, all cost functions with a maximum flow rate were assigned an auxiliary cost function of the form in Eq. (452). The parameters of the auxiliary function were derived so as to most closely approximate the original cost function. Note that the scaling factor for each process unit is between 0 and 1, exclusive, so each cost function will be a concave, monotonically increasing function of the working flow rate.

### Example 5.3

### Deterministic Global Optimization Strategies

To solve the process synthesis with simultaneous heat, power, and water integration problem, a branch-and-bound global optimization algorithm (Misener et al., 2010, 2011; Misener & Floudas, 2010, which are incorporated herein by reference as if fully set forth) is introduced as described below. At each node in the branch-and-bound tree, a mixed-integer linear relaxation of the mathematical model is solved using CPLEX 12.3 (CPLEX, 2009) and then the node is branched to create two children nodes. The solution pool feature of CPLEX is utilized during the solution of the relaxed model to generate a set of 150 distinct points, each of which is used as a candidate starting point to solve the original model. For each starting point, the current binary variable values are fixed and the resulting NLP is minimized using CONOPT 3.15A. If the solution to the NLP is less than the current upper bound, then the upper bound is replaced with the NLP solution value. At each step, all nodes that have a lower bound that is within an 6 tolerance of the current upper bound ((LB_{node})/(UB)≧1−∈) are eliminated from the tree. Termination of the algorithm is reached if all nodes in the branch-and-bound tree have been processed or if 100 CPU hours have passed. Upon completion of the algorithm, the model lower bound (represented as the minimum value for the lower bound of all nodes yet to be processed) and the best upper bound are reported.

The following sections detail specific strategies employed at the root node and general strategies used at each node of the branch-andbound tree.

### Example 5.3.1

### Bilinear Term Underestimation

Each of the bilinear terms is derived from the product of two continuous, non-negative variables. The tightest possible relaxation of the bilinear term z=x·y is defined using the envelopes that define the convex and concave hulls, as shown in Eqs. (456)-(459), where x^{L}≦x≦x^{U} and y^{L}≦y≦y^{U}.

z≧x·y^{L}+x^{L}·y−x^{L}·y^{L} (456)

z≧x·y^{U}+x^{U}·y−x^{U}·y^{U} (457)

z≧x·y^{L}+x^{U}·y−x^{U}·y^{L} (458)

z≧x·y^{U}+x^{L}·y−x^{L}·y^{U} (459)

The envelopes defined by these four equations are dependent on the size of the domain of x and y, and a disjunctive program can be formulated by partitioning one of the variables (x) into N_{P} segments. In the disjunctive program, the N_{P} segments on the range [x^{L}, x^{U}] are each bounded by [x^{L}+a·(n_{P}−1), x^{L}+a·n_{P}]∀nₚ∈{1, . . . , N_{P}} where a=x^{U}−c^{L}/N_{P}. The partitioning scheme described below will activate exactly one n_{P} so that the feasible space corresponding to the relaxation of x·y goes from the large parallelogram defined by the convex hull over the entire region (Eqs. (456) and (457)) to a substantially smaller parallelogram. Once the methodology behind the partitioning scheme has been outlined, the following sections will detail how the partitioning scheme is applied to each of the bilinear terms in the CBGTL model.

### Example 5.3.1.1

### Logarithmic Partitioning Scheme

The logarithmic partitioning scheme for piecewise linear relaxation utilizes three additional variable sets where the number of variables introduced will scale logarithmically with the number of partitions for each bilinear term. The number of logarithmic terms, N_{L}, is defined as N_{L}=log₂ N_{P}. Binary switches (λ_{nL}), continuous switches (Δy_{nL}), and continuous slacks (sl_{nL}) are then defined over all n_{L}∈{1, . . . , NL} as follows:

λₙ_{_{L}}∈{0,1}

Δyₙ_{_{L}}∈[0,y^{U}−y^{L}]

slₙ_{_{L}}∈[0,y^{U}−y^{L}]

Note that there is a one-to-one mapping between the activation of a one of the N_{P} segments and a combination of the N_{L} binary variables. The N_{L} elements of λ will activate or deactivate based on the binary representation of the largest grid point that is less than x, as shown in Eq. (460).

$${x}^{L} + {\sum }_{{n}_{L} = 1}^{{N}_{L}} {2}^{{n}_{L} - 1} \cdot a \cdot {λ}_{{n}_{L}} \leq x \leq a + {x}^{L} + {\sum }_{{n}_{L} = 1}^{{N}_{L}} {2}^{{n}_{L} - 1} \cdot a \cdot {λ}_{{n}_{L}} \qquad ( 460 )$$

The Δy_{nL} variables should be equal to (y−y^{L}) for each active λ_{nL}, as restricted by Eqs. (461)-(463).

Δyₙ_{_{L}}≦(y^{U}−y^{L})·λₙ_{_{L}} (461)

Δyₙ_{_{L}}=(y−y^{L})−slₙ_{_{L}} (462)

0≦slₙ_{_{L}}≦(y^{U}−y^{L})·(1−λₙ_{_{L}}) (463)

Using the Using the definitions provided above, a logarithmic partitioning scheme that is equivalent to the previously desired disjunctive program is introduced using Eqs. (464)-(467).

$$\begin{matrix} z \geq x \cdot {y}^{L} + {x}^{L} \cdot ( y - {y}^{L} ) + {\sum }_{{n}_{L} = 1}^{{N}_{L}} a \cdot {2}^{{n}_{L} - 1} \cdot Δ {y}_{{n}_{L}} & ( 464 ) \\ z \geq x \cdot {y}^{U} + ( {x}^{L} + a ) \cdot ( y - {y}^{U} ) + {\sum }_{{n}_{L} = 1}^{{N}_{L}} a \cdot {2}^{{n}_{L} - 1} \cdot ( Δ {y}_{{n}_{L}} - ( {y}^{U} - {y}^{L} ) \cdot {λ}_{{n}_{L}} ) & ( 465 ) \\ z \leq x \cdot {y}^{L} + ( {x}^{L} + a ) \cdot ( y - {y}^{L} ) + {\sum }_{{n}_{L} = 1}^{{N}_{L}} a \cdot {2}^{{n}_{L} - 1} \cdot Δ {y}_{{n}_{L}} & ( 466 ) \\ z \geq x \cdot {y}^{U} + {x}^{L} \cdot ( y - {y}^{U} ) + {\sum }_{{n}_{L} = 1}^{{N}_{L}} a \cdot {2}^{{n}_{L} - 1} \cdot ( Δ {y}_{{n}_{L}} - ( {y}^{U} - {y}^{L} ) \cdot {λ}_{{n}_{L}} ) & ( 467 )\end{matrix}$$

### Example 5.3.1.2

### Flash Units Phase Equilibrium

For the flash units, all outlet streams are defined as (u, u′)∈UC_{Fl} and all species within those streams are defined as (u, u′, s)∈S_{Fl}. To construct the grid of total flow rates for the flash units, the total stream flow rate (N_{u,u′}^{T}) variables are partitioned into a grid using N_{P} segments of equal length using Eq. (468) and the lower (N_{u,u′}^{T-LB}) and upper (N_{u,u′}^{T-UB}) bounds of the flow rate.

$${N}_{u , {u}^{′}}^{T - gr} = \frac{{N}_{u , {u}^{′}}^{T - UB} - {N}_{u , {u}^{′}}^{T - LB}}{{N}_{P}} ∀ ( u , {u}^{′} ) ∈ {UC}_{Fl} \qquad ( 468 )$$

Binary variables, λ_{u,u′,n}_{_{L}}^{PE}, are introduced to activate only one domain segment using Eqs. (469) and (470). For this study, the number of partitions selected was equal to 4 (N_{P}=4), so the number of binary variables introduced is equal to 2 (N_{L}=2).

$$\begin{matrix} {N}_{u , {u}^{′}}^{T} \geq {N}_{u , {u}^{′}}^{T - LB} + {\sum }_{{n}_{L} = 1}^{{N}_{L}} {2}^{{n}_{L} - 1} \cdot {N}_{u , {u}^{′}}^{T - gr} \cdot {λ}_{u , {u}^{′} , {n}_{L}}^{PE} ∀ ( u , {u}^{′} ) ∈ {UC}_{Fl} & ( 469 ) \\ {N}_{u , {u}^{′}}^{T} \leq {N}_{u , {u}^{′}}^{T - LB} + {\sum }_{{n}_{L} = 1}^{{N}_{L}} ( {2}^{{n}_{L} - 1} \cdot {N}_{u , {u}^{′}}^{T - gr} \cdot {λ}_{u , {u}^{′} , {n}_{L}}^{PE} ) + {N}_{u , {u}^{′}}^{T - gr} ∀ ( u , {u}^{′} ) ∈ {UC}_{Fl} & ( 470 )\end{matrix}$$

Continuous variables Δx_{u,u′,s,n}_{_{L}}^{S} and sl_{u,u′,n}_{_{L}}^{PE} are used for the concentration variables x_{u,u′,s}^{S} and are equal to zero in all inactive intervals and equal to (x_{u,u′,s}^{S}−x_{u,u′,s}^{S-LB}) in all active intervals. This is enforced using Eqs. (471)-(473) where x_{u,u′,s}^{S-UB} and x_{u,u′,s}^{S-LB} are the upper and lower bounds, respectively, on the concentration variables.

Δx_{u,u′,s,n}_{_{L}}^{S}≦(x_{u,u′,s}^{S-UB}−x_{u,u′,s}^{S-LB})·λ_{u,u′,n}_{_{L}}^{PE}∀(u,u′,s)∈S_{Fl},n_{L}=1, . . . ,N_{L} (471)

Δx_{u,u′,s,n}_{_{L}}^{S}=(x_{u,u′,s}^{S}−x_{u,u′,s}^{S-LB})·sl_{u,u′,n}_{_{L}}^{PE}∀(u,u′,s)∈S_{Fl},n_{L}=1, . . . ,N_{L} (472)

sl_{u,u′,s,n}_{_{L}}^{PE}≦(x_{u,u′,s}^{S-UB}−x_{u,u′,s}^{S-LB})·(1−λ_{u,u′,n}_{_{L}}^{PE})∀(u,u′,s)∈S_{Fl},n_{L}=1, . . . ,N_{L} (473)

The relaxation of the bilinear term, defined as w_{u,u′,s}^{PE} is placed in the phase equilibrium constraint as Eq. (51).

w_{u,u′,s}^{PE}−N_{u,u′,s}^{S}=0∀(u,u′,s)∈S_{Fl} (474)

The w_{u,u′,s}^{PE} variable is restricted in the following constraints.

$$\begin{matrix}{w}_{u , {u}^{′} , s}^{PE} \{ \begin{matrix}\geq {N}_{u , {u}^{′}}^{T} \cdot {x}_{u , u , s}^{S - LB} + {N}_{u , {u}^{′}}^{T - LB} \cdot ( {x}_{u , {u}^{′} , s}^{S} - {x}_{u , {u}^{′} , s}^{S - LB} ) + \\ {\sum }_{{n}_{L} = 1}^{{N}_{L}} {N}_{u , {u}^{′}}^{T - gr} \cdot {2}^{{n}_{L} - 1} \cdot Δ {x}_{u , {u}^{′} , s , {n}_{L}}^{S} \\ \begin{matrix}\geq {N}_{u , {u}^{′}}^{T} \cdot {x}_{u , {u}^{′} , s}^{S - UB} + ( {N}_{u , {u}^{′}}^{T - LB} + {N}_{u , {u}^{′}}^{T - gr} ) \cdot ( {x}_{u , {u}^{′} , s}^{S} - {x}_{u , {u}^{′} , s}^{S - UB} ) + \\ {\sum }_{{n}_{L} = 1}^{{N}_{L}} {N}_{u , {u}^{′}}^{T - gr} \cdot {2}^{{n}_{L} - 1} \cdot ( Δ {x}_{u , {u}^{′} , s , {n}_{L}}^{S} - ( {x}_{u , {u}^{′} , s}^{S - UB} - {x}_{u , {u}^{′} , s}^{S - UB} ) \cdot {λ}_{u , {u}^{′} , {n}_{L}}^{PE} )\end{matrix} \\ \begin{matrix}\leq {N}_{u , {u}^{′}}^{T} \cdot {x}_{u , {u}^{′} , s}^{S - LB} + ( {N}_{u , {u}^{′}}^{T - LB} + {N}_{u , {u}^{′}}^{T - gr} ) \cdot ( {x}_{u , {u}^{′} , s}^{S} - {x}_{u , {u}^{′} , s}^{S - LB} ) + \\ {\sum }_{{n}_{L} = 1}^{{N}_{L}} {N}_{u , {u}^{′}}^{T - gr} \cdot {2}^{{n}_{L} - 1} \cdot Δ {x}_{u , {u}^{′} , s , {n}_{L}}^{S}\end{matrix} \\ \begin{matrix}\leq {N}_{u , {u}^{′}}^{T} \cdot {x}_{u , {u}^{′} , s}^{S - UB} + {N}_{u , {u}^{′}}^{T - LB} \cdot ( {x}_{u , {u}^{′} , s}^{S} - {x}_{u , {u}^{′} , s}^{S - UB} ) + \\ {\sum }_{{n}_{L} = 1}^{{N}_{L}} {N}_{u , {u}^{′}}^{T - gr} \cdot {2}^{{n}_{L} - 1} \cdot ( Δ {x}_{u , {u}^{′} , s , {n}_{L}}^{S} - ( {x}_{u , {u}^{′} , s}^{S - UB} - {x}_{u , {u}^{′} , s}^{S - LB} ) \cdot {λ}_{u , {u}^{′} , {n}_{L}}^{PE} )\end{matrix}\end{matrix} & ( 475 ) \\ ∀ ( u , {u}^{′} , s ) ∈ {S}_{Fl} & \end{matrix}$$

### Example 5.3.1.3

### Splitter Units Stream Splitting

For the splitter units, all outlet streams are defined as (u, u′)∈UC_{Sp} and all species flow rates into the splitter are defined as (uₗ, u, s)∈S_{Sp}. The sp_{u,u′} variables are partitioned using 8 segments (N_{P}=8) of equal length (Eq. (476)), where sp_{u,u′}^{UB} and sp_{u,u′}^{UB} are the upper and lower bounds on the split fractions, respectively.

$${sp}_{u , {u}^{′}}^{gr} = \frac{{sp}_{u , {u}^{′}}^{UB} - {sp}_{u , {u}^{′}}^{LB}}{{N}_{P}} ∀ ( u , {u}^{′} ) ∈ {UC}_{Sp} \qquad ( 476 )$$

Binary variables, λ_{u,u′,n}_{_{L}}^{SS}, are introduced to activate only one domain segment using Eqs. (477)-(478). The number of binary variables introduced for each split fraction variable is equal to 3.

$$\begin{matrix} {sp}_{u , {u}^{′}} \geq {sp}_{u , {u}^{′}}^{LB} + {\sum }_{{n}_{L} = 1}^{{N}_{L}} {2}^{{n}_{L} - 1} \cdot {sp}_{u , {u}^{′}}^{gr} \cdot {λ}_{u , {u}^{′} , {n}_{L}}^{SS} ∀ ( u , {u}^{′} ) ∈ {UC}_{Sp} & ( 477 ) \\ {sp}_{u , {u}^{′}} \leq {sp}_{u , {u}^{′}}^{LB} + {\sum }_{{n}_{L} = 1}^{{N}_{L}} ( {2}^{{n}_{L} - 1} \cdot {sp}_{u , {u}^{′}}^{gr} \cdot {λ}_{u , {u}^{′} , {n}_{L}}^{SS} ) + {sp}_{u , {u}^{′}}^{gr} ∀ ( u , {u}^{′} ) ∈ {UC}_{Sp} & ( 478 )\end{matrix}$$

Continuous variables ΔNᵤ_{ₗ}_{,u,s,n}_{_{L}}^{S} and slᵤ_{ₗ}_{,u,n}_{_{L}}^{SS} are used for the species flow rate variables, as shown in Eqs. (479)-(481) where Nᵤ_{ₗ},u,s^{S-UB} and Nᵤ_{ₗ}_{,u,s}^{S-LB} are the upper and lower bounds, respectively, on the concentration variables.

ΔNᵤ_{ₗ}_{,u,s,n}_{_{L}}^{S}≦(Nᵤ_{ₗ}_{,u,s}^{S-UB}−Nᵤ_{ₗ}_{,u,s}^{S-LB})·λᵤ_{ₗ}_{,u,n}_{_{L}}^{SS}∀(uₗ,u,s)∈S_{Sp},n_{L}=1, . . . ,N_{L} (479)

ΔNᵤ_{ₗ}_{,u,s,n}_{_{L}}^{S}=(Nᵤ_{ₗ}_{,u,s}^{S}−Nᵤ_{ₗ}_{,u,s}^{S-LB})·slᵤ_{ₗ}_{,u,n}_{_{L}}^{SS}∀(uₗ,u,s)∈S_{Sp},n_{L}=1, . . . ,N_{L} (480)

slᵤ_{ₗ}_{,u,s,n}_{_{L}}^{SS}≦(Nᵤ_{ₗ}_{,u,s}^{S-UB}−Nᵤ_{ₗ}_{,u,s}^{S-LB})·(λᵤ_{ₗ}_{,u,n}_{_{L}}^{SS})∀(uₗ,u,s)∈S_{Sp},n_{L}=1, . . . ,N_{L} (481)

The bilinear relaxation, w_{u,u′,s}^{SS} is placed in the equilibrium constraint as Eq. (482).

w_{u,u′,s}^{CE}−N_{u,u′,s}^{S}=0∀(uₗ,u,s)∈S_{Sp},(u,u′)∈U_{Sp} (482)

Note that there is only one input stream (uₗ, u) to each splitter unit. Therefore, the bilinear relaxation variables do not need to be indexed over u_{I}. The w_{u,u′,s}^{SS} variable is restricted as follows:

$$\begin{matrix}{w}_{u , {u}^{′} , s}^{SS} \{ \begin{matrix}\geq {sp}_{u , {u}^{′}} \cdot {N}_{{u}_{I} , u , s}^{S - LB} + {sp}_{u , {u}^{′}}^{LB} \cdot ( {N}_{{u}_{I} , u , s}^{S} - {N}_{{u}_{I} u , s}^{S - LB} ) + \\ {\sum }_{{n}_{L} = 1}^{{N}_{L}} {sp}_{u , {u}^{′}}^{gr} \cdot {2}^{{n}_{L} - 1} \cdot Δ {N}_{{u}_{I} , u , s , {n}_{L}}^{S} \\ \begin{matrix}\geq {sp}_{u , {u}^{′}} \cdot {N}_{{u}_{I} , u , s}^{S - UB} + ( {sp}_{u , {u}^{′}}^{LB} + {sp}_{u , {u}^{′}}^{gr} ) \cdot ( {N}_{{u}_{I} , u , s}^{S} - {N}_{{u}_{I} u , s}^{S - UB} ) + \\ {\sum }_{{n}_{L} = 1}^{{N}_{L}} {sp}_{u , {u}^{′}}^{gr} \cdot {2}^{{n}_{L} - 1} \cdot ( Δ {N}_{{u}_{I} , u , s , {n}_{L}}^{S} - ( {N}_{{u}_{I} , u , s}^{S - UB} - {N}_{{u}_{I} , u , s}^{S - LB} ) \cdot {λ}_{u , {u}^{′} , {n}_{L}}^{SS} )\end{matrix} \\ \begin{matrix}\leq {sp}_{u , {u}^{′}} \cdot {N}_{{u}_{I} , u , s}^{S - LB} + ( {sp}_{u , {u}^{′}}^{LB} + {sp}_{u , {u}^{′}}^{gr} ) \cdot ( {N}_{{u}_{I} , u , s}^{S} - {N}_{{u}_{I} u , s}^{S - LB} ) + \\ {\sum }_{{n}_{L} = 1}^{{N}_{L}} {sp}_{u , {u}^{′}}^{gr} \cdot {2}^{{n}_{L} - 1} \cdot Δ {N}_{{u}_{I} , u , s , {n}_{L}}^{S}\end{matrix} \\ \begin{matrix}\leq {sp}_{u , {u}^{′}} \cdot {N}_{{u}_{I} , u , s}^{S - UB} + {sp}_{u , {u}^{′}}^{LB} \cdot ( {N}_{{u}_{I} , u , s}^{S} - {N}_{{u}_{I} u , s}^{S - UB} ) + \\ {\sum }_{{n}_{L} = 1}^{{N}_{L}} {sp}_{u , {u}^{′}}^{gr} \cdot {2}^{{n}_{L} - 1} \cdot ( Δ {N}_{{u}_{I} , u , s , {n}_{L}}^{S} - ( {N}_{{u}_{I} , u , s}^{S - UB} - {N}_{{u}_{I} , u , s}^{S - LB} ) \cdot {λ}_{u , {u}^{′} , {n}_{L}}^{SS} )\end{matrix}\end{matrix} & ( 483 ) \\ ∀ ( {u}_{l} , u , s ) ∈ {S}_{Sp} , ( u , {u}^{′} ) ∈ {U}_{Sp} & \end{matrix}$$

### Example 5.3.1.4

### Reactor Units—Chemical Equilibrium

All streams that are restricted by chemical equilibrium are labeled as (u, u′)∈UC_{CE}. Each of the bilinear terms is defined as the product of two species flow rates, N_{u,u′,s}^{S}. The set of stream flow rates that is used as the “x” variable is (u, u′, s)∈s_{CE}^{y} and the set of stream flow rates used as the “y” variable is (u, u′,s)∈s_{CE}^{y}. For this study, the H₂ and H₂O species were chosen as the “x” variables for the water-gas-shift equilibrium. In the auto-thermal reactor, CO is also used as an “x” variable to handle the bilinear term created in Eq. (441). The N_{u,u′,s}^{S} variables are partitioned using 8 segments (N_{P}=8) of equal length (Eq. (484)), where N_{u,u′,s}^{S-UB} and N_{u,u′,s}^{S-LB} are the upper and lower bounds on the species flow rates, respectively.

$${N}_{u , {u}^{′} , s}^{S - gr} = \frac{{N}_{u , {u}^{′} , s}^{S - UB} - {N}_{u , {u}^{′}}^{S - LB}}{{N}_{P}} ∀ ( u , {u}^{′} , s ) ∈ {S}_{CE}^{x} \qquad ( 484 )$$

Three binary variables, λ_{u,u′,s,n}_{_{L}}^{CE}, are introduced for each species variable and activate the domain segments according to Eqs. (485) and (486).

$$\begin{matrix}{N}_{u , {u}^{′} , s}^{S} \geq {N}_{u , {u}^{′} , s}^{S - LB} + {\sum }_{{n}_{L} = 1}^{{N}_{L}} {2}^{{n}_{L} - 1} \cdot {N}_{u , {u}^{′} , s}^{S - gr} \cdot {λ}_{u , {u}^{′} , s , {n}_{L}}^{CE} ∀ ( u , {u}^{′} , s ) ∈ {S}_{CE}^{x} & ( 485 ) \\ {N}_{u , {u}^{′} , s}^{S} \geq {N}_{u , {u}^{′} , s}^{S - LB} + {\sum }_{{n}_{L} = 1}^{{N}_{L}} {2}^{{n}_{L} - 1} \cdot {N}_{u , {u}^{′} , s}^{S - gr} \cdot {λ}_{u , {u}^{′} , s , {n}_{L}}^{CE} ∀ ( u , {u}^{′} , s ) ∈ {S}_{CE}^{x} & ( 486 )\end{matrix}$$

Continuous variables ΔN_{u,u′,s,n}_{_{L}}^{S} and sl_{u,u′,s,n}_{_{L}}^{CE} are used for the species flow rate variables, as shown in Eqs. (487)-(488).

ΔN_{u,u′,s,n}_{_{L}}^{S}≦(N_{u,u′,s}^{S-UB}−N_{u,u′,s}^{S-LB})·λ_{u,u′,s,n}_{_{L}}^{CE}∀(u,u′,s)∈S_{CE}^{y},n_{L}=1, . . . ,N_{L} (487)

ΔN_{u,u′,s,n}_{_{L}}^{S}=(N_{u,u′,s}^{S}−N_{u,u′,s}^{S-LB})·sl_{u,u′,s,n}_{_{L}}^{CE}∀(u,u′,s)∈S_{CE}^{y},n_{L}=1, . . . ,N_{L} (488)

sl_{u,u′,s,n}_{_{L}}^{CE}≦(N_{u,u′,s}^{S-UB}−N_{u,u′,s}^{S-LB})·(1−λ_{u,u′,s,n}_{_{L}}^{CE})∀(u,u′,s)∈S_{CE}^{y},n_{L}=1, . . . ,N_{L} (489)

The bilinear relaxation, w_{u,u′,s,s′}^{CE}, is placed in the water-gas-shift equilibrium constraint as shown in Eqs. (490) and (491). The relaxation constraints for the auto-thermal reactor are detailed in Eqs. (492) and (493) for CH₄ steam reforming, in Eqs. (494) and (495) for C₂H₂ steam reforming, in Eqs. (496) and (497) for C₂H₄ steam reforming, and in Eqs. (498) and (499) for C₂H₆ steam reforming.

$$\begin{matrix}{w}_{u , {u}^{′} , CO , {H}_{2} O}^{CE} - {K}_{u , {u}^{′} , {u}_{WGS}}^{WGS} \cdot {w}_{u , {u}^{′} , {CO}_{2} , {H}_{2}}^{CE} - {N}_{u , {u}^{′} , CO}^{S - UB} \cdot {N}_{u , {u}^{′} , {H}_{2} O}^{S - UB} \cdot ( 1 - {y}_{{u}_{WGS}} ) \leq 0 ∀ ( u , {u}^{′} , {u}_{WGS} ) ∈ {U}_{WGS} & ( 490 ) \\ {K}_{u , {u}^{′} , {u}_{WGS}}^{WGS} \cdot {w}_{u , {u}^{′} , {CO}_{2} , {H}_{2}}^{CE} - {w}_{u , {u}^{′} , CO , {H}_{2} O}^{CE} - {K}_{u , {u}^{′} , {u}_{WGS}}^{WGs} \cdot {N}_{u , {u}^{′} , {CO}_{2}}^{S - UB} \cdot {N}_{u , {u}^{′} , {H}_{2}}^{S - UB} \cdot ( 1 - {y}_{{u}_{WGS}} ) \leq 0 ∀ ( u , {u}^{′} , {u}_{WGS} ) ∈ {U}_{WGS} & ( 491 ) \\ {N}_{u , {u}^{′} , CO}^{S} \cdot {N}_{u , {u}^{′} , {H}_{2}}^{S 3} - {K}_{u , {CH}_{4}}^{SR} \cdot {w}_{u , {u}^{′} , {CH}_{4} , {H}_{2} O}^{CE} - {N}_{u , {u}^{′} , CO}^{S - UB} \cdot {N}_{u , {u}^{′} , {H}_{2}}^{S - UB 3} \cdot ( 1 - {y}_{{u}_{ATR}} ) \leq 0 ∀ ( u , {u}^{′} , {u}_{ATR} ) ∈ {U}_{ATR} & ( 492 ) \\ {K}_{u , {CH}_{4}}^{SR} \cdot {w}_{u , {u}^{′} , {CH}_{4} , {H}_{2} O}^{CE} - {N}_{u , {u}^{′} , CO}^{S} \cdot {N}_{u , {u}^{′} , {H}_{2}}^{S 3} - {K}_{u , {CH}_{4}}^{SR} \cdot {N}_{u , {u}^{′} , {CH}_{4}}^{S - UB} \cdot ( 1 - {y}_{{u}_{ATR}} ) \leq 0 ∀ ( u , {u}^{′} , {u}_{ATR} ) ∈ {U}_{ATR} & ( 493 ) \\ {w}_{u , {u}^{′} , {C}_{2} {H}_{2} , {H}_{2} O}^{CE} - \frac{{K}_{u , {CH}_{4}}^{SR}}{{K}_{u , {C}_{2} , {H}_{2}}^{SR}} \cdot {w}_{u , {u}^{′} , {CH}_{4} , CO}^{CE} - {N}_{u , {u}^{′} , {C}_{2} {H}_{2}}^{S - UB} \cdot {N}_{u , {u}^{′} , {H}_{2} O}^{S - UB} \cdot ( 1 - {y}_{{u}_{ATR}} ) \leq 0 ∀ ( u , {u}^{′} , {u}_{ATR} ) ∈ {U}_{ATR} & ( 494 ) \\ \frac{{K}_{u , {CH}_{4}}^{SR}}{{K}_{u , {C}_{2} , {H}_{2}}^{SR}} \cdot {w}_{u , {u}^{′} , {CH}_{4} , CO}^{CE} - {w}_{u , {u}^{′} , {C}_{2} {H}_{2} , {H}_{2} O}^{CE} - \frac{{K}_{u , {CH}_{4}}^{SR}}{{K}_{u , {C}_{2} , {H}_{2}}^{SR}} \cdot {N}_{u , {u}^{′} , {CH}_{4}}^{S - UB} \cdot {N}_{u , {u}^{′} , CO}^{S - UB} \cdot ( 1 - {y}_{{u}_{ATR}} ) \leq 0 ∀ ( u , {u}^{′} , {u}_{ATR} ) ∈ {U}_{ATR} & ( 495 ) \\ {N}_{u , {u}^{′} , {C}_{2} {H}_{2}}^{S} - \frac{{K}_{u , {C}_{2} {H}_{2}}^{SR}}{{K}_{u , {C}_{2} {H}_{4}}^{SR}} \cdot {w}_{u , {u}^{′} , {C}_{2} {H}_{2} , {H}_{2}}^{CE} - {N}_{u , {u}^{′} , {C}_{2} {H}_{2}}^{S - UB} \cdot ( 1 - {y}_{{u}_{ATR}} ) \leq 0 ∀ ( u , {u}^{′} , {u}_{ATR} ) ∈ {U}_{ATR} & ( 496 ) \\ \frac{{K}_{u , {C}_{2} {H}_{2}}^{SR}}{{K}_{u , {C}_{2} {H}_{4}}^{SR}} \cdot {w}_{u , {u}^{′} , {C}_{2} {H}_{2} , {H}_{2}}^{CE} - {N}_{u , {u}^{′} , {C}_{2} {H}_{4}}^{S} - \frac{{K}_{u , {C}_{2} {H}_{2}}^{SR}}{{K}_{u , {C}_{2} {H}_{4}}^{SR}} \cdot {N}_{u , {u}^{′} , {C}_{2} {H}_{2} , {H}_{2}}^{S - UB} - {N}_{u , {u}^{′} , {H}_{2}}^{S - UB} \cdot ( 1 - {y}_{{u}_{ATR}} ) \leq 0 ∀ ( u , {u}^{′} , {u}_{ATR} ) ∈ {U}_{ATR} & ( 497 ) \\ {N}_{u , {u}^{′} , {C}_{2} {H}_{6}}^{S} - \frac{{K}_{u , {C}_{2} {H}_{4}}^{SR}}{{K}_{u , {C}_{2} {H}_{6}}^{SR}} \cdot {w}_{u , {u}^{′} , {C}_{2} {H}_{4} , {H}_{2}}^{CE} - {N}_{u , {u}^{′} , {C}_{2} {H}_{6}}^{S - UB} \cdot ( 1 - {y}_{{u}_{ATR}} ) \leq 0 ∀ ( u , {u}^{′} , {u}_{ATR} ) ∈ {U}_{ATR} & ( 498 ) \\ \frac{{K}_{u , {C}_{2} {H}_{4}}^{SR}}{{K}_{u , {C}_{2} {H}_{6}}^{SR}} \cdot {w}_{u , {u}^{′} , {C}_{2} {H}_{4} , {H}_{2}}^{CE} - {N}_{u , {u}^{′} , {C}_{2} {H}_{6}}^{S} - \frac{{K}_{u , {C}_{2} {H}_{4}}^{SR}}{{K}_{u , {C}_{2} {H}_{6}}^{SR}} \cdot {N}_{u , {u}^{′} , {C}_{2} {H}_{4}}^{S - UB} \cdot {N}_{u , {u}^{′} , {H}_{2}}^{S - UB} \cdot ( 1 - {y}_{{u}_{ATR}} ) \leq 0 ∀ ( u , {u}^{′} , {u}_{ATR} ) ∈ {U}_{ATR} & ( 499 )\end{matrix}$$

The w_{u,u′,s,s′}^{CE} variables are restricted using Eq. (500), where the set (u, u′, s, s′)∈S_{CE}^{x,y} is defined as all combinations of (u, u′, s)∈S_{CE}^{y} and (u, u′, s′)∈S_{CE}^{x} that form a product of two species flow rates.

$$\begin{matrix}{w}_{u , {u}^{′} , s}^{PE} \{ \begin{matrix}\geq {N}_{u , {u}^{′} , s}^{S} \cdot {N}_{u , {u}^{′} , s}^{S - LB} + {N}_{u , {u}^{′} , s}^{S - LB} \cdot ( {N}_{u , {u}^{′} , s}^{S} - {N}_{u , {u}^{′} , s}^{S - LB} ) + \\ {\sum }_{{n}_{L} = 1}^{{N}_{L}} {N}_{u , {u}^{′} , {s}^{′}}^{S - gr} \cdot {2}^{{n}_{L} - 1} \cdot Δ {N}_{u , {u}^{′} , s , {n}_{L}}^{S} \\ \begin{matrix}\geq {N}_{u , {u}^{′} , {s}^{′}}^{T} \cdot {N}_{u , {u}^{′} , s}^{S - UB} + ( {N}_{u , {u}^{′} , {s}^{′}}^{S - LB} + {N}_{u , {u}^{′} , {s}^{′}}^{S - gr} ) \cdot ( {N}_{u , {u}^{′} , s}^{S} - {N}_{u , {u}^{′} , s}^{S - UB} ) + \\ {\sum }_{{n}_{L} = 1}^{{N}_{L}} {N}_{u , {u}^{′} , {s}^{′}}^{S - gr} \cdot {2}^{{n}_{L} - 1} \cdot ( Δ {N}_{u , {u}^{′} , s , {n}_{L}}^{S} - ( {N}_{u , {u}^{′} , s}^{S - UB} - {N}_{u , {u}^{′} , s}^{S - UB} ) \cdot {λ}_{u , {u}^{′} , {s}^{′} , {n}_{L}}^{CE} )\end{matrix} \\ \begin{matrix}\leq {N}_{u , {u}^{′} , {s}^{′}}^{S} \cdot {N}_{u , {u}^{′} , s}^{S - LB} + ( {N}_{u , {u}^{′} , {s}^{′}}^{S - LB} + {N}_{u , {u}^{′} , {s}^{′}}^{S - gr} ) \cdot ( {N}_{u , {u}^{′} , s}^{S} - {N}_{u , {u}^{′} , s}^{S - LB} ) + \\ {\sum }_{{n}_{L} = 1}^{{N}_{L}} {N}_{u , {u}^{′} , {s}^{′}}^{S - gr} \cdot {2}^{{n}_{L} - 1} \cdot Δ {N}_{u , {u}^{′} , s , {n}_{L}}^{S}\end{matrix} \\ \begin{matrix}\leq {N}_{u , {u}^{′} , {s}^{′}}^{S} \cdot {N}_{u , {u}^{′} , s}^{S - UB} + {N}_{u , {u}^{′} , {s}^{′}}^{S - LB} \cdot ( {N}_{u , {u}^{′} , s}^{S} - {N}_{u , {u}^{′} , s}^{S - UB} ) + \\ {\sum }_{{n}_{L} = 1}^{{N}_{L}} {N}_{u , {u}^{′} , {s}^{′}}^{S - gr} \cdot {2}^{{n}_{L} - 1} \cdot ( Δ {N}_{u , {u}^{′} , s , {n}_{L}}^{S} - ( {N}_{u , {u}^{′} , s}^{S - UB} - {N}_{u , {u}^{′} , s}^{S - LB} ) \cdot {λ}_{u , {u}^{′} , {s}^{′} , {n}_{L}}^{CE} )\end{matrix}\end{matrix} & ( 500 ) \\ ∀ ( u , {u}^{′} , s , {s}^{′} ) ∈ {S}_{CE}^{x , y} & \end{matrix}$$

Note that Eqs. (492) and (493) both contain a quadrilinear term which may be underestimated using a convex relaxation, a bilinear term relaxation and a successive trilinear term relaxation, or three successive auxiliary bilinear terms. In this study, it was found that three successive bilinear relaxations provided a tight relaxation due to the piecewise linear partitioning that is employed on each of the bilinear terms. The auxiliary variable, w_{u,u′,s,s′}^{CE}, isl used to model the bilinear combination of the CO and H₂ species flow rates (i.e., N_{u,u′,CO}^{S}·N_{u,u′,H}_{₂}^{S}). This auxiliary variable is added to the mathematical model along with the corresponding binary, λ_{u,u′,H}_{₂}_{,n}_{_{L}}^{CE}, and continuous variables, ΔN_{u,u′,CO,n}_{_{L}}^{S} and sl_{u,u′,CO,n}_{_{L}}^{CE} using the above equations. Eqs. (492) and (493) are then reformulated as Eqs. (501) and (502).

w_{u,u′,CO,H}_{₂}^{CE}·N_{u,u′,H}_{₂}^{S}^{²}−K_{u,CH}_{₄}^{SR}·w_{u,u′,CH}_{₄}_{,H}_{₂}_{O}^{CE}−N_{u,u′,CO}^{S-UB}·N_{u,u′,H}_{₂}^{S-UB}^{²}·(1−yᵤ_{_{ATR}})≦0∀(u,u′,u_{ATR})∈U_{ATR} (501)

K_{u,CH}_{₄}^{SR}·w_{u,u′,CH}_{₄}_{,H}_{₂}_{O}^{CE}−w_{u,u′,CO,H}_{₂}^{CE}·N_{u,u′,H}_{₂}^{S}^{²}−K_{u,CH}_{₄}^{SR}·N_{u,u′,CH}_{₄}^{S-UB}·N_{u,u′,H}_{₂}_{O}^{S-UB}·(1−yᵤ_{_{ATR}})≦0∀(u,u′,u_{ATR})∈U_{ATR} (502)

Two auxiliary species (CO—H₂ and CO—H₂—H₂) are then defined to exist within the auto-thermal reactor effluent that are designed to only participate in the relaxation equations. These species flow rates are set equal to the auxiliary relaxation variables (Eqs. (503) and (504)) so that the above formulation can be applied iteratively until no nonlinear terms remain.

N_{u,u′,CO—H}_{₂}^{S}=w_{u,u′,CO,H}_{₂}^{CE}∀(u,u′,u_{ATR})∈U_{ATR} (503)

N_{u,u′,CO—H}_{₂}_{—H}_{₂}^{S}=w_{u,u′,CO—H}_{₂}_{,H}_{₂}^{CE}∀(u,u′,u_{ATR})∈U_{ATR} (504)

That is, after the second iteration (reducing the trilinear term to a bilinear term), the model relaxation will change from Eqs. (501) and (502) to Eqs. (505) and (506).

w_{u,u′,CO—H}_{₂}_{,H}_{₂}^{CE}·N_{u,u′,H}_{₂}^{S}−K_{u,CH}_{₄}^{SR}·w_{u,u′,CH}_{₄}_{,H}_{₂}_{O}^{CE}−N_{u,u′,H}_{₂}^{S-UB}^{³}·(1−yᵤ_{_{ATR}})≦0∀(u,u′,u_{ATR})∈U_{ATR} (505)

K_{u,CH}_{₄}^{SR}·w_{u,u′,CH}_{₄}_{,H}_{₂}_{O}^{CE}−w_{u,u′,CO—H}_{₂}_{,H}_{₂}^{CE}·N_{u,u′,H}_{₂}^{S}−K_{u,CH}_{₄}^{SR}·N_{u,u′,CH}_{₄}^{S-UB}·N_{u,u′,H}_{₂}_{O}^{S-UB}·(1−yᵤ_{_{ATR}})≦0∀(u,u′,u_{ATR})∈U_{ATR} (506)

A final iteration will yield Eqs. (507) and (508), which represent the final form of the relaxation used in the mathematical model.

w_{u,u′,CO—H}_{₂}_{—H}_{₂}_{,H}_{₂}^{CE}−K_{u,CH}_{₄}^{SR}·w_{u,u′,CH}_{₄}_{,H}_{₂}_{O}^{CE}−N_{u,u′,CO}^{S UB}·N_{u,u′,H}_{₂}^{S UB}^{³}·(1−yᵤ_{_{ATR}})≦0∀(u,u′,u_{ATR})∈U_{ATR} (507)

K_{u,CH}_{₄}^{SR}·w_{u,u′,CH}_{₄}_{,H}_{₂}_{O}^{CE}−w_{u,u′,CO—H}_{₂}_{—H}_{₂}_{,H}_{₂}^{CE}−K_{u,CH}_{₄}^{SR}·N_{u,u′,CH}_{₄}^{S-UB}·N_{u,u′,H}_{₂}_{O}^{S-UB}·(1−yᵤ_{_{ATR}})≦0∀(u,u′,u_{ATR})∈U_{ATR} (508)

### Example 5.3.2

### Concave Cost Function Underestimation

To underestimate the cost functions, a linear partitioning scheme was utilized which introduces special-ordered-set (SOS2) variables, y_{i,u}^{l}, to define each piece. The MILP solver CPLEX supports the use of these variables and has the capability to handle their special structure when optimizing the relaxation model (CPLEX, 2009). For a given ordered set i, the SOS2 variables are 0-1 continuous and are constrained such that only two variables may be active (value greater than zero) and these two variables must be at adjacent elements (i.e., i and i+1). Given a continuous piecewise linear function, the SOS2 variables may then be used to define the function by Eqs. (509) and (510). That is, a series of coordinates (s_{i,u}^{C}, Inc_{i,u}^{C}) are determined for each cost function and can be used to construct a piecewise linear approximation of the original function. Given a working flow rate of a unit sᵤ (sᵤ=(Sᵤ)/(Sᵤ^{o})), Eq. (510) will define the affine piece of the approximation that bounds the flow rate (i.e., s_{i-1,u}^{C}≦sᵤ≦s_{i,u}^{C}). The values of the SOS2 variables y_{i-1,u}^{l} and y_{i,u}^{l} will define the investment cost of the unit based on the linear approximation in Eq. (509).

$$\begin{matrix}{Inv}_{u} = {\sum }_{( i , u ) ∈ {U}_{Inv}^{P}} {Inv}_{i , u}^{C} \cdot {y}_{i , u}^{I} ∀ u ∈ {U}_{Inv} & ( 509 ) \\ {s}_{u} = {\sum }_{( i , u ) ∈ {U}_{Inv}^{P}} {s}_{i , u}^{C} \cdot {y}_{i , u}^{I} ∀ u ∈ {U}_{Inv} & ( 510 )\end{matrix}$$

The unit investment cost values, Invᵤ, will play a direct role in the objective function of the model, so adequate approximation of the concave cost functions is essential for a tight bound on the objective function. Given a cost function of the form Invᵤ=Cᵤ^{o}·sᵤ^{sf}^{ᵤ} and a point along the curve (s_{i,u}^{C}, Inv_{i,u}^{C}), a linear underestimation may be constructed between points (s_{i,u}^{C}, Inv_{i,u}^{C}) and (s_{i+1,u}^{C}, Inc_{i+1,u}^{C}) such that the maximum error between the original cost function and the linear underestimation is at most a given percent, errᵤ. That is, a function of the form Invᵤ^{L}=mᵤ·sᵤ+bᵤ is desired such that the linear function intersects with the original cost function at the two desired points and that

$$\frac{{Inv}_{u}^{L}}{{Inv}_{u}} \geq 1 - {err}_{u}$$

for all sᵤ∈[s_{i,u}^{C},s_{i+1,u}^{C}].

The difference, Diffᵤ, between the original function and the linear underestimation is given using Eq. (516).

$${Diff}_{u} = {C}_{u}^{O} \cdot {s}_{u}^{{sf}_{u}} - \frac{{Inv}_{i + 1 , u}^{C} - {Inv}_{i , u}^{C}}{{s}_{i + 1 , u}^{C} - {s}_{i , u}^{C}} \cdot ( {s}_{u} - {s}_{i , u}^{C} ) - {Inv}_{i , u}^{C} ∀ u ∈ {U}_{Inv} \qquad ( 511 )$$

The maximum error between the two functions will occur at point sᵤ=sᵤ^{M} when the derivative of the function is equal to zero, shown in Eq. (512) where

$${m}_{u} = \frac{{Inv}_{i + 1 , u}^{C} - {Inv}_{i , u}^{C}}{{s}_{i + 1 , u}^{C} - {s}_{i , u}^{C}} .$$

This can be rearranged to find the value for sᵤ^{M}, as described in Eq. (513). The value for the maximum offset, errᵤ, can then be defined using Eq. (514).

$$\begin{matrix}0 = {sf}_{u} \cdot {C}_{u}^{o} \cdot {( {s}_{u}^{M} )}^{{sf}_{u} - 1} - {m}_{u} ∀ u ∈ {U}_{Inv} & ( 512 ) \\ {s}_{u}^{M} = {( \frac{{m}_{u}}{{sf}_{u} \cdot {C}_{u}^{o}} )}^{\frac{1}{{sf}_{u} - 1}} ∀ u ∈ {U}_{Inv} & ( 513 ) \\ \frac{{m}_{u} \cdot ( {s}_{u}^{M} - {s}_{i , u}^{C} ) + {Inv}_{i , u}^{C}}{{C}_{u}^{o} + {( {s}_{u}^{M} )}^{{sf}_{u}}} = 1 - {err}_{u} ∀ u ∈ {U}_{Inv} & ( 514 )\end{matrix}$$

The error calculated in Eq. (514) will ultimately be a function of the right intersection point (s_{i+1,u}^{C}, Inc_{i+1,u}^{C}) for the linear function and can be determined either using MATLAB or a guess-and-solve iteration approach. The complete piecewise linear underestimation can therefore be constructed by beginning with the lower bound on the sᵤ variable as the initial s_{0,u}^{C} point. The strategy above can be used to find the s_{1,u}^{C} point that will ensure that the maximum offset error between s_{0,u}^{C} and s_{1,u}^{C} is equal to errᵤ. The point s_{2,u}^{C} is then determined by utilizing the value for s_{1,u}^{C} as the left point for the next iteration and the process continues until a calculated point s_{i,u}^{C} is greater than the upper bound for sᵤ. Once this occurs, the final calculated point s_{i,u}^{C} is set equal to the upper bound. The set of (s_{i,u}^{C}, Inc_{i,u}^{C}) values are used within Eqs. (509) and (510) to ensure that the maximum error associated with any point along the cost function approximation is less than errᵤ. For this study, a majority of the process units were selected to have a maximum error of 10%. The units expected to contribute the most to the overall cost (i.e., coal gasifier, steam turbines, air separation unit, and wax hydrocracker) have a maximum error of 5%. Note that since the investment cost is anticipated to account for approximately 30-35% of the overall cost, the maximum anticipated error between the best feasible solution and the lower bound will be in the range of 2-4%. It is possible to reduce this anticipated error by reducing the value for erru for the process units. However, the calculation of the lower bound will become increasingly complex due to the inclusion of additional y_{i,u}^{l} SOS2 variables needed to define the linear underestimators. The values for the unit investment cost errors defined above represent an acceptable balance between solution quality and computational efficiency.

### Example 5.3.3

### Calculation of Initial Upper Bound

At the root node of the branch-and-bound tree, it is critical to identify (1) a high-quality upper bound and (2) tight ranges on the variables that will be branched on in the tree. Therefore, the initial step of the global optimization approach is to calculate a high-quality upper bound from a local solution of the problem. Using the solution pool feature of CPLEX (CPLEX, 2009), 150 points are generated as candidate starting points to a non-linear optimization (NLP) solver. To expedite the determination of the initial points, all bilinear terms are modeled using the standard convex envelopes (N_{P}=1) and the concave cost functions are modeled using a single linear underestimator. Each nonlinear term is therefore relaxed without binary variables, and while this does not provide a tight lower bound, it does serve to find a large array of distinct initial points (i.e., different topological scenarios) within a short period of time. At each iteration, the values of the binary variables for the starting point are fixed and the resulting NLP is solved to find a local solution. The lowest objective value of all of the local solution is retained as the initial upper bound on the final solution value (Eq. (515)).

Cost≦Cost_{UB} (515)

### Example 5.3.4

### Optimality Based Bounds Tightening

Given the restriction on the upper bound shown in Eq. (515), rigorous bounds may be then determined for several problem variables. That is, Eq. (516) can be used as an objective function to find the minimum and maximum possible value of certain species molar flow rates, total molar flow rates, and unit working flow rates for the superstructure. For a given iteration, all parameter coefficients (C_{u,u′,s}^{N}^{^{S}}, C_{u,u′}^{N}^{^{T}}, and Cᵤ^{S}) are set to zero except for the coefficient pertaining to the variable of interest, which is set to one. The objective function reduces to the variable of interest which is minimized and subsequently maximized to find the lower and upper bounds of this variable. Note that this step is capable of significantly reducing the variable bounds of each process variable. The bounds tightening procedure is specifically targeted at the variables that will appear in bilinear terms for phase equilibrium (N_{u,u′}^{T}), stream splitting (N_{u,u′,s}^{S}), chemical equilibrium (N_{u,u′,s}^{S}), or the cost functions (Sᵤ).

min/max C_{u,u′,s}^{N}^{^{S}}·N_{u,u′,s}^{S}+C_{u,u′}^{N}^{^{T}}·N_{u,u′}^{T}+Cᵤ^{S}·Sᵤ (516)

This procedure is preformed using the complete set of linear underestimators detailed earlier. That is each model solved using Eq. (516) as the objective is a MILP with the appropriate piecewise linear underestimators for the bilinear terms and the cost functions. This was found to provide better solutions as opposed to solving a quicker, relaxed version of the problem that changes all 0-1 binary variables to 0-1 continuous variables. After each solution is determined, the tightened bounds on the variables will lead to tighter relaxations and therefore to tighter ranges for the variables of Eq. (516). In fact, multiple passes may be made across the entire set of variables with the end result being tighter variable bounds for each successive pass. After a certain point, the decrease in variable bounds will start to be rather small while the time required for solution of the MILP will increase. In this study, two passes were made through the aforementioned set of variables, which was found to be a proper balance between the time required to run the bounds tightening and the overall decrease in variable bounds. The maximum run time for each solver call was set to 1 min, which prevented any single call from using a significant amount of computational time. Upon completion of the MILP solver, the best possible relaxed value of the objective was taken as the final value for the variable bound. If a problem was solved to complete optimality, this would also be equal to value for the optimal incumbent solution.

### Example 5.3.5

### Chemical Equilibrium Species Ratios

For all species participating in the chemical equilibrium, it is important to determine the maximum or minimum ratio that the species molar flow rate can have with respect to another species. This will aid in the feasibility based bounds tightening strategy that is outlined below. A series of ratio values, Ratᵢ, are determined over an indexed set, i∈TR, where the leftmost value is set to zero (Rat₀=0) and the rightmost bound is set to an arbitrarily large value (Rat_{TR}=1×10⁵). The values are selected such that Ratᵢ>Ratᵢ₋₁ for each index i. For two species s and s′ within a given stream (u, u′), the ratio of the molar flow of species s to species s′ will be bounded within two consecutive values based on the activation of the binary variable yᵢ^{R}, as shown in Eqs. (518)-(520). If the value of yᵢ^{R} is zero, then the constraints in Eqs. (518) and (519) will be redundant. Activation of only one binary variable is enforced using Eq. (520). The resulting MILP model can be solved using CPLEX using the objective function in Eq. (517) to try to find the maximum and minimum possible ratios. Upon maximization of the objective, the value for MaxRat_{u,u′,s,s′} is equal to Ratᵢ while after minimization of the objective, the value of MinRat_{u,u′,s,s′} is equal to Ratᵢ₋₁.

$$\begin{matrix}min / max {\sum }_{i ∈ TR} {Rat}_{i} \cdot {y}_{i}^{R} & ( 517 ) \\ {N}_{u , {u}^{′} , s}^{S} \geq {Rat}_{i - 1} \cdot [ {N}_{u , {u}^{′} , {s}^{′}}^{S} - {N}_{u , {u}^{′} , {s}^{′}}^{S \text{-} UB} \cdot ( 1 - {y}_{i}^{R} ) ] ∀ i ∈ TR & ( 518 ) \\ {N}_{u , {u}^{′} , {s}^{′}}^{S} \leq {Rat}_{i} \cdot {N}_{u , {u}^{′} , {s}^{′}}^{S} + {N}_{u , {u}^{′} , s}^{S \text{-} UB} \cdot ( 1 - {y}_{i}^{R} ) ∀ i ∈ TR & ( 519 ) \\ {\sum }_{i ∈ TR} {y}_{i}^{R} = 1 & ( 520 )\end{matrix}$$

### Example 5.3.6

### Branching Strategies

Upon solving a relaxation at a given node using the logarithmically partitioned bilinear underestimators and the piecewise linear cost function underestimators, a variable is selected for branching and the value used to construct the two children nodes is determined. Only the variables used in the bilinear terms will be candidates for branching. Note that the cost function variables could be used for partitioning, but branching on these variables is not beneficial as adding more terms to the piecewise underestimators (i.e., reduce the error between the relaxation and the original function). The variables selected for partitioning will be either (i) the stream flow rate variables participating in chemical equilibrium or (ii) the split fraction variables for the stream splitters. It should be noted that the branching scheme detailed below is capable of using any of the variables participating in the bilinear terms. However, the two variable sets mentioned above were frequently selected as branching candidates and provided better partitioning of the search space than the other variables. Due to the binary range partitioning implemented for the “x” variables in the bilinear terms, it was generally found that branching on these variables provided better partitioning than on the “y” variables. Therefore, only the H₂, H₂O, and CO species for (i) the stream flow rate variables will be selected as branching candidates. The set of stream flow rate variable indices used for branching is called S_{CE}^{x-br} and the set of split fraction variable indices used for branching is called UC_{SS}^{br}.

After generating the optimal solution for the lower bound using CPLEX (2009), the variable N_{u,u′,s}^{S} or sp_{u,u′} is selected for branching that has the greatest discrepancy between the auxiliary and original problem variables (Adjiman, Androulakis, & Floudas, 1998; Adjiman, Dallwig, Floudas, & Neumaier, 1998; Audet, Hansen, Jaumard, & Savard, 2000; Misener & Floudas, 2010), as shown in Eq. (521).

$${argmax}_{( u , {u}^{′} , {s}^{′} ) ∈ {S}_{CE}^{x \text{-} br}} {\sum }_{( u , {u}^{′} , s , {s}^{′} ) ∈ {S}_{CE}^{xy}} \vert {w}_{u , {u}^{′} , s , {s}^{′}}^{CE} - {N}_{u , {u}^{′} , s}^{\\ S} \cdot {N}_{u , {u}^{′} , {s}^{′}}^{S} \vert + {\sum }_{( {u}_{I} , {u}^{⋁} , {s}^{⋁} ) ∈ {S}_{SS}} \vert {w}_{{u}^{⋁} , {{u}^{⋁}}^{′} , s}^{SS} - {N}_{{{u}^{⋁}}_{I} , {u}^{⋁} , s}^{S} \cdot {sp}_{{u}^{⋁} , {{u}^{⋁}}^{′}} \vert ( {u}^{⋁} , {{u}^{⋁}}^{′} ) ∈ {UC}_{SS}^{br} \qquad ( 521 )$$

Once the appropriate variable is selected, the point within the variable range is chosen as the branching location to form the two children nodes. For a given variable x∈[x^{L}, x^{U}] with solution value x′, the location for branching, x^{br}, was determined using the formula in Eq. (522), where λ_{C} is a parameter that selects the branch point partially between the halfway point of the variable range and the optimal solution value. In this study, λ_{C}=0.1 to emphasize a partition that is close to the optimal point, and has shown to provide some advantages to partitioning at the optimal point when the variable range is small and the branch-and-bound tree becomes larger (Misener & Floudas, 2010). For a more comprehensive discussion of branching strategies, the reader is directed to previously published works (Adjiman, Androulakis, et al., 1998; Floudas, 2000; Tawarmalani & Sahinidis, 2002).

x^{br}=λ_{C}·0.5·(x^{L}+x^{U})+(1−λ_{C})·x′ (522)

### Example 5.3.7

### Feasibility Based Bounds Tightening

Prior to determining the lower bound at a node, a series of checks can be made on each variable bound to ensure that the bound does not conflict with a constraint that exists within the model. Once a variable is selected for branching and the range is partitioned, the new lower or upper bounds on the variable may alter the lower and upper bounds of other variables. For the split fraction variables, the lower bound (sp_{u,u′}^{LB}) may be adjusted if one minus the sum of the upper bounds (sp_{u,u″}^{UB}) of all other split fraction variables from that unit are greater than the current lower bound (Eq. (523)). The upper bound of a split fraction variable may be adjusted if one minus the sum of the lower bounds of the other unit split fraction variables are lower than the current upper bound (Eq. (524)).

$$\begin{matrix}{sp}_{u , {u}^{′}}^{LB} = max ( {sp}_{u , {u}^{′}}^{LB} , 1 - {{\sum }_{( u , {u}^{″} ) ∈ {UC}_{SS}}}^{{u}^{″} \neq {u}^{′}} {sp}_{u , {u}^{″}}^{UB} ) ∀ ( u , {u}^{′} ) ∈ {UC}_{SS} & ( 523 ) \\ {sp}_{u , {u}^{′}}^{UB} = min ( {sp}_{u , {u}^{′}}^{UB} , 1 - {{\sum }_{( u , {u}^{″} ) ∈ {UC}_{SS}}}^{{u}^{″} \neq {u}^{′}} {sp}_{u , {u}^{″}}^{LB} ) ∀ ( u , {u}^{′} ) ∈ {UC}_{SS} & ( 524 )\end{matrix}$$

Feasibility checks on the stream flow rate variables are enforced using knowledge of the maximum/minimum possible ratio of the molar flow each species related to another. For any species participating in chemical equilibrium (s_{CE}=s_{CE}^{x}∪s_{CE}^{y}), the lower bound on the molar flow rate (N_{u,u′,s}^{S-LB}) may be adjusted if the product of the lower bound of another species (N_{u,u′,s′}^{S-LB}) and the minimum ratio between the two species is greater than the current lower bound (Eq. (525)). Similarly, the upper bound of a species molar flow rate (N_{u,u′,s}^{S-UB}) may be adjusted using the upper bound of another species and the maximum ratio (Eq. (526)).

N_{u,u′,s}^{S-LB}=max(N_{u,u′,s}^{S-LB},MinRat_{u,u′,s,s′}·N_{u,u′,s′}^{S-LB})∀(u,u′,s),(u,u′,s′)∈S_{CE} (525)

N_{u,u′,s}^{S-UB}=min(N_{u,u′,s}^{S-UB},MaxRat_{u,u′,s,s′}·N_{u,u′,s′}^{S-UB})∀(u,u′,s),(u,u′,s′)∈S_{CE} (526)

At each stage of the branch-and-bound tree, the bounds on the variables could be tightened using an optimality based routine. However, no significant benefit was seen when this strategy was implemented due to the large computational time required to implement this procedure for all variables within the nonconvex terms (approximately 4 CPU hours).

### Example 5.4

### Computational Results of Twelve Case Studies

The proposed global optimization routine was used to analyze twelve distinct case studies using perennial biomass (switchgrass), lowvolatile bituminous coal (Illinois #6), and natural gas as feedstocks. To examine the effects of potential economies of size on the final liquid fuels price, three distinct plant capacities were examined to represent a small, medium, or large capacity hybrid energy plant. Based on current petroleum refinery capacities, representative sizes of 10 thousand barrels per day (TBD), 50 TBD, and 200 TBD were chosen, respectively. The trade-offs for CO2 handling including sequestration, venting, and reaction to form CO via the reverse water-gas-shift reaction were examined by enforcing different levels of feedstock carbon conversion to liquid fuels. Conversion rates of at least 25%, 50%, 75%, and 95% were enforced for each of the three plant capacities, resulting in the twelve case studies that will be presented. The overall greenhouse gas emission target for each case study is set to have a 50% reduction from petroleum based processes (Baliban et al., 2011, 2012). The cost parameters used for CBGTL process are listed in Table 62.

TABLE 62
Cost parameters (2009 $) for the CBGTL refinery.

| Item | Cost | Item | Cost |
|---|---|---|---|
| Coal (LV | $93.41/short ton | Biomass | $139.97/dry metric |
| Bituroinous) | | (Switchgrass) | ton |
| Natural gas | $5.39/TSCF | Freshwater | $0.50/metric ton |
| Butanes | $1.84/gallon | Propanes | $1.78/gallon |
| Electricity | $0.07/kWhr | CO₂ sequestration | $20/metric ton |

TSCF, thousand standard cubic feet.

Once the global optimization algorithm is completed, the resulting process topology provides (i) the operating conditions and working fluid flow rates of the heat engines, (ii) the amount of electricity produced by the engines, (iii) the amount of cooling water needed for the engines, and (iv) the location of the pinch points denoting the distinct subnetworks. Given this information, the minimum heat exchanger matches necessary to meet specifications (i)-(iv) are calculated as previously described (Baliban et al., 2011; Floudas, 1995; Floudas, Ciric, & Grossmann, 1986). Upon solution of the minimum matches model, the heat exchanger topology with the minimum annualized cost can be found using the superstructure methodology (Elia et al., 2010; Floudas, 1995; Floudas et al., 1986). The investment cost of the heat exchangers is added to the investment cost calculated within the process synthesis model to obtain the final investment cost for the superstructure.

### Example 5.4.1

### Global Optimization Framework

The case studies were each tested on a single computer containing 8 Intel Xeon 2.83 GHz processors and shared memory parallelization. The lower bound of each node in the branch-and-bound tree was solved using CPLEX and eight parallel threads (CPLEX, 2009), while the upper bound was solved serially using CONOPT. The computational time for each node was largely spent computing the lower bound, so the serial computation of the upper bound did not hinder the progress of the branch-and-bound tree. Parallelization of the entire branch and bound algorithm using a message passing interface and a shared memory system on a Beowulf cluster will be the study of a future investigation. The original MINLP model contains 15,439 continuous variables, 30 binary variables, 15,406 equality constraints, 230 inequality constraints, 274 bilinear terms, 1 quadrilinear term, and 60 concave power functions.

For each lower bound, the bilinear terms were relaxed using a logarithmic partitioning scheme with 4 partitions for the phase equilibrium terms and 8 partitions for the remaining terms. The quadrilinear term was relaxed using three successive bilinear term relaxations with 8 partitions each. This led to the introduction of an additional 139 binary variables, 1793 continuous variables, and 2747 inequality constraints to fully define the partitioning scheme. The total amount of constraints does not include the introduction of the auxiliary variables in the original mathematical model, since these constraints will simply replace the nonlinear constraints of the original model. The concave functions were underestimated using a piecewise linear scheme using 2-5 SOS2 variables for each function, leading to a total of 108 SOS2 variables. The 120 equality constraints generated from the underestimation replace the 60 nonlinear constraints of the original model. The complete MILP model for the lower bound therefore contains a total of 17,232 continuous variables, 169 binary variables, 108 SOS2 variables, 15,466 equality constraints, and 2977 inequality constraints. At each node of the branch-and-bound tree, the MILP model was terminated upon reaching optimality or after 1800 s (30 min) of computational time. For each upper bound, a multi-start technique was utilized where the binary variables are fixed and the resulting NLP was solved to optimality. The resulting NLP model contained 15,439 continuous variables, 15,406 equality constraints, and 230 inequality constraints along with the same amount of nonconvex terms as the original MINLP model.

The results of the entire global optimization algorithm are shown in Table 63. For each case study, the computational results are shown after completion of the root node and upon termination of the solver. The termination criterion for the algorithm was set to allow the algorithm to run for 100 CPU hours (3.6×105 s). After 100 CPU hours, the quality of both the lower and upper bounds did not improve for any of the twelve case studies. At the root node, an upper bound for the model is initially calculated, followed by the optimality based bounds tightening and the calculation of the first relaxation (lower bound). From Table 63, it is evident that a majority of the computational effort at the root node is spent calculating the upper bound (5466-7047 s for all calls to the solver) and the bounds tightening (25,337-28,286 s) while the least amount of effort is spent on calculating the relaxation (1156-1484 s). This is in contrast to the remaining nodes of the branch-and-bound tree where the majority of time (>80%) is spent calculating the relaxation while the balance is spent calculating the feasibility tightening (<1%) and the upper bound (<20%). The computational effort to calculate the upper bound is higher at the root node because the multi-start technique uses 150 initial points, while only 10 initial points are used at subsequent nodes. Progression of the branch-and-bound tree was not enhanced when an increased number of initial points was used at the children nodes of the tree, though the generation of a high-quality feasible points (upper bound) at the root node does have a noticeable effect on the tree. The value for the upper bound found at the root node will influence the optimality based bounds tightening and therefore the quality of relaxations generated at all nodes throughout the tree. The selection of 150 initial points at the root node was chosen as a proper balance between the solution quality obtained at the root node and the computational effort required. That is, as the number of initial points was increased, the upper bound obtained at the root node showed little or no change. If the number of initial points at the root node was decreased, the quality of the upper bound at the root node began to decrease and had an adverse effect on the entire global optimization algorithm.

Upon completion of the root node, the optimality gap between the lower and upper bounds ranges from 12.35% to 36.10% throughout the various case studies. To enhance the quality of the relaxation at the root node, the number of partitions used for the bilinear terms and the concave functions could be used. In fact, if the number of partitions was increased to 32 for each bilinear term and the error in the cost functions was at most 2% (4-9 SOS2 variables per function), then the relaxation at the root node can be enhanced enough to reduce the gap to between (9-16%) for all case studies. The gap still seen with this tight relaxation implies that a branch-and-bound tree should be used to provide a tighter guarantee of optimality. Note that when the branch-and-bound algorithm is used for this large partitioning scheme, the computational time at the root node ranges from 95,000 to 110,000 s due to a slight increase for the optimality based bounds tightening (30,000-35,000 s) and a substantial increase for the relaxation (59,000-65,000 s).

TABLE 63
Branch and bound results for the twelve case studies.

| Feed. | Root node | | | | | | | Termination | | | |
|---|---|---|---|---|---|---|---|---|---|---|---|
| Conv. % | Relaxation | UB | Gap | t_{UB} (s) | t_{OB} (s) | t_{R} (s) | Nodes | LB | UB | % Gap | Total CPU (s) |

Small plant capacity (10 TBD)

| | | | | | | | | | | | |
|---|---|---|---|---|---|---|---|---|---|---|---|
| 25 | 8.32 | 13.02 | 36.10 | 5785 | 28,286 | 1480 | 313 | 11.93 | 12.54 | 4.86 | 360,000 |
| 50 | 9.01 | 13.98 | 35.55 | 6942 | 27,450 | 1293 | 301 | 12.42 | 13.01 | 4.54 | 360,000 |
| 75 | 18.32 | 24.51 | 25.25 | 7047 | 26,388 | 1269 | 294 | 20.65 | 22.03 | 6.25 | 360,000 |
| 95 | 26.32 | 30.00 | 12.35 | 5466 | 27,692 | 1287 | 318 | 28.65 | 29.54 | 4.27 | 360,000 |

Medium plant capacity (50 TBD)

| | | | | | | | | | | | |
|---|---|---|---|---|---|---|---|---|---|---|---|
| 25 | 8.49 | 12.79 | 33.62 | 6284 | 28,170 | 1482 | 285 | 11.28 | 12.03 | 6.23 | 360,000 |
| 50 | 9.03 | 13.04 | 30.75 | 5994 | 26,829 | 1156 | 302 | 11.75 | 12.85 | 8.56 | 360,000 |
| 75 | 18.43 | 22.52 | 18.16 | 6501 | 25,337 | 1234 | 314 | 20.74 | 21.43 | 3.22 | 360,000 |
| 95 | 23.35 | 30.12 | 22.48 | 5628 | 26,794 | 1478 | 320 | 27.21 | 28.56 | 4.73 | 360,000 |

Large plant capacity (200 TBD)

| | | | | | | | | | | | |
|---|---|---|---|---|---|---|---|---|---|---|---|
| 25 | 7.71 | 12.00 | 35.75 | 5698 | 26,442 | 1224 | 271 | 10.43 | 11.32 | 7.86 | 360,000 |
| 50 | 8.61 | 12.94 | 33.23 | 6095 | 26,835 | 1176 | 281 | 11.00 | 11.97 | 8.02 | 360,000 |
| 75 | 15.32 | 20.84 | 26.49 | 6832 | 27,148 | 1484 | 282 | 17.51 | 19.11 | 8.21 | 360,000 |
| 95 | 23.41 | 27.21 | 13.97 | 5697 | 26,470 | 1189 | 296 | 25.31 | 26.49 | 4.40 | 360,000 |

The total time for finding the upper bound (tUB), the optimality based bounds tightening (tOB), and relaxation (tR) are listed for the root node along with the final value of the relaxation (in $/GJ). The total number of nodes used within the branch-and-bound tree before termination is listed along with the find lower (LB) and upper (UB) bound (in $/GJ) and the gap at termination. Note that all runs were terminated when the total CPU time reached 100 h (3.6×105 s).

The benefit of the branch-and-bound tree for the twelve case studies is evident when looking at the best feasible solution (upper bound) and the relaxation (lower bound) at termination. For all case studies, the gap ranges between 3.22% and 8.56% (Table 63). This is substantially reduced from the gap at the root node due to both an increase in the relaxation throughout the branch-and-bound tree and a decrease in the upper bound throughout the tree. A decrease in the upper bound implies that a better feasible solution was found during the branch-and-bound process than was achieved during the root node. In fact, several better feasible solutions were found for most of the case studies during the progression of the tree. This implies the existence of local minima throughout the mathematical model landscape make it difficult for the solver to find other feasible solutions that have a lower objective value. Note that a different initialization technique could be employed at the root node that would allow the solver to more efficiently find feasible solutions that are obtained later in the branch-and-bound tree. However, the mathematical guarantee of the optimality of these solutions is not known until the global optimization algorithm is used.

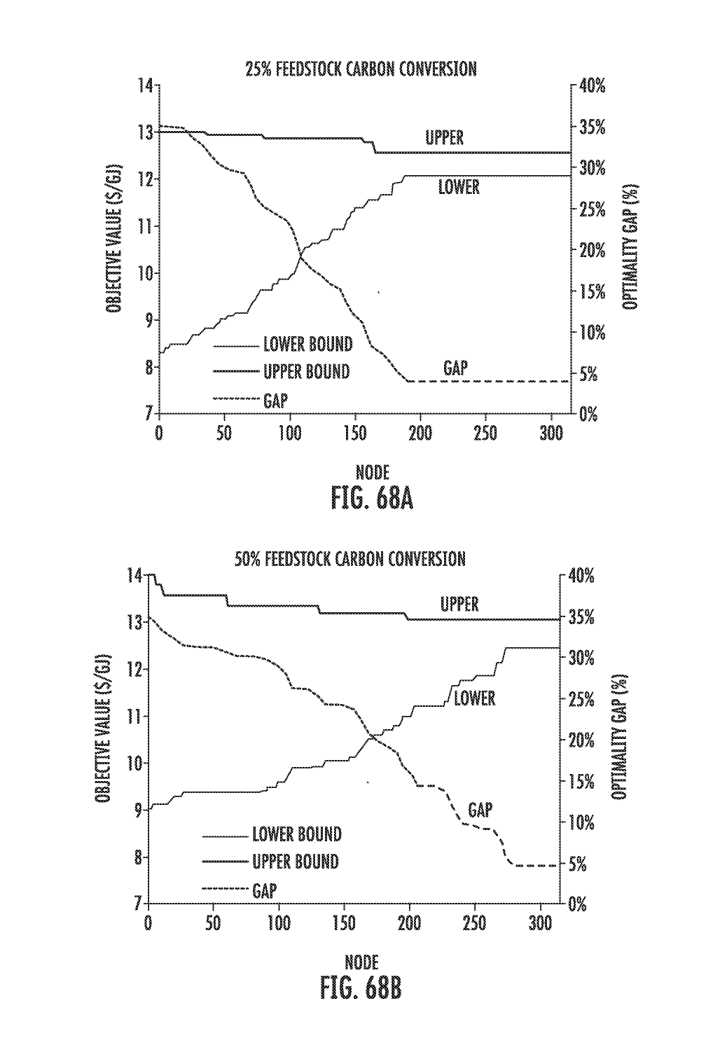

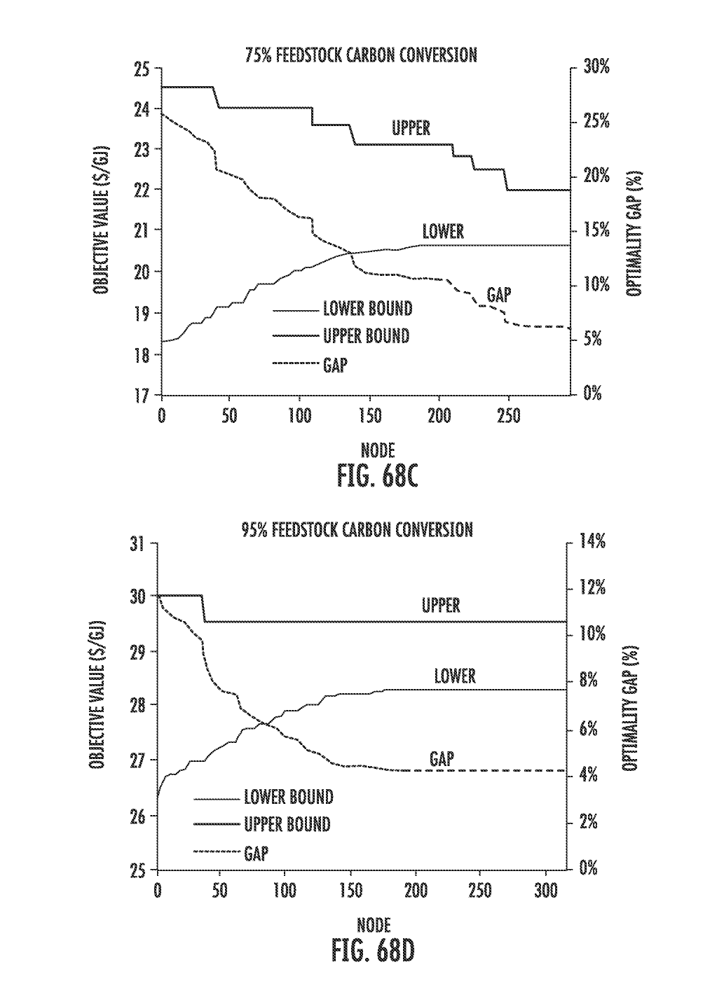

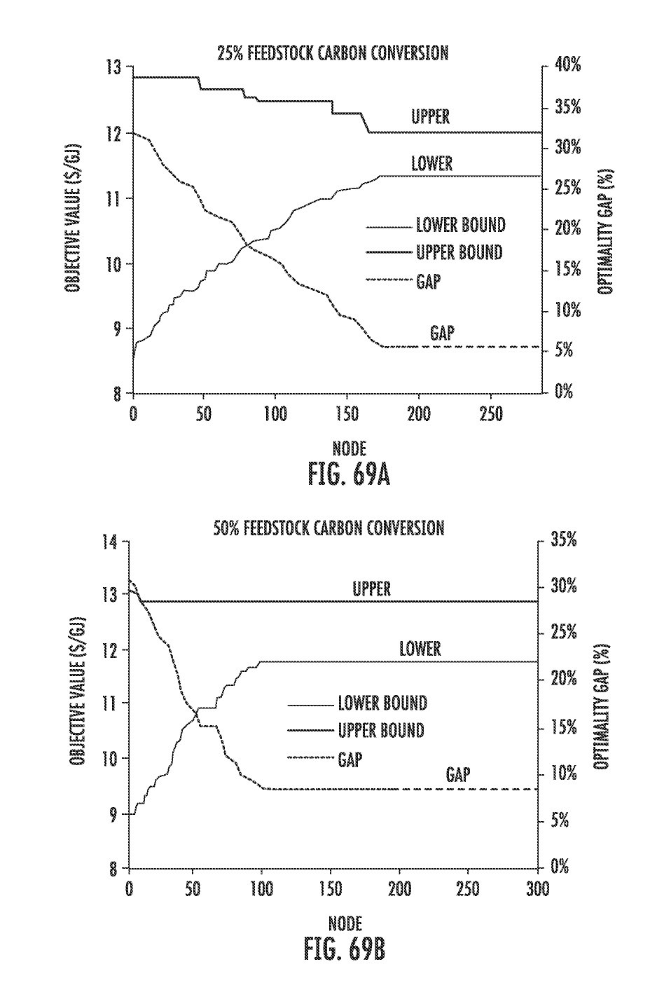

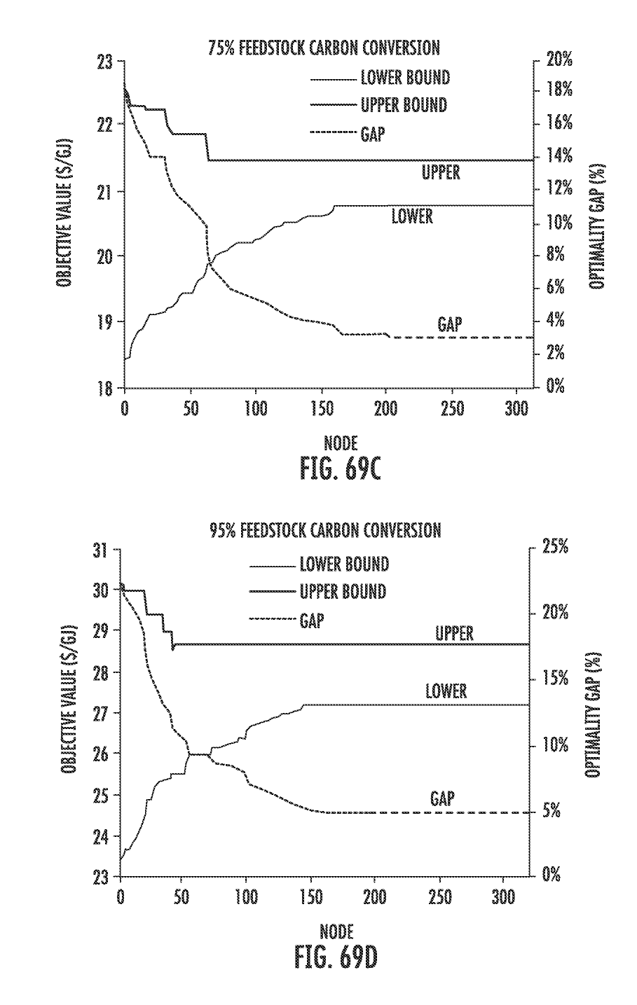

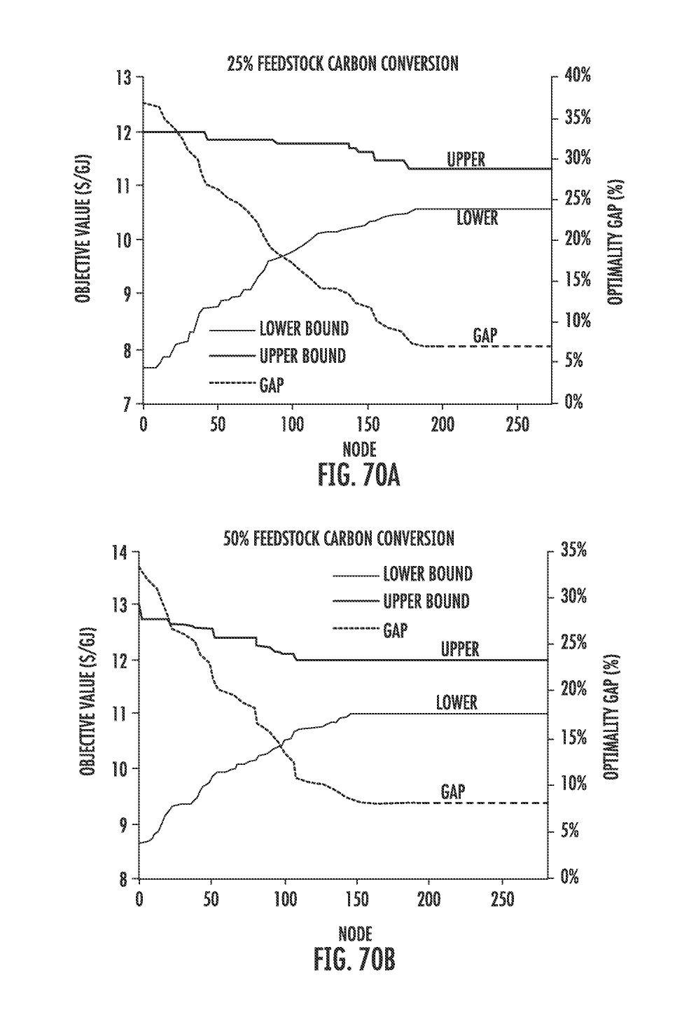

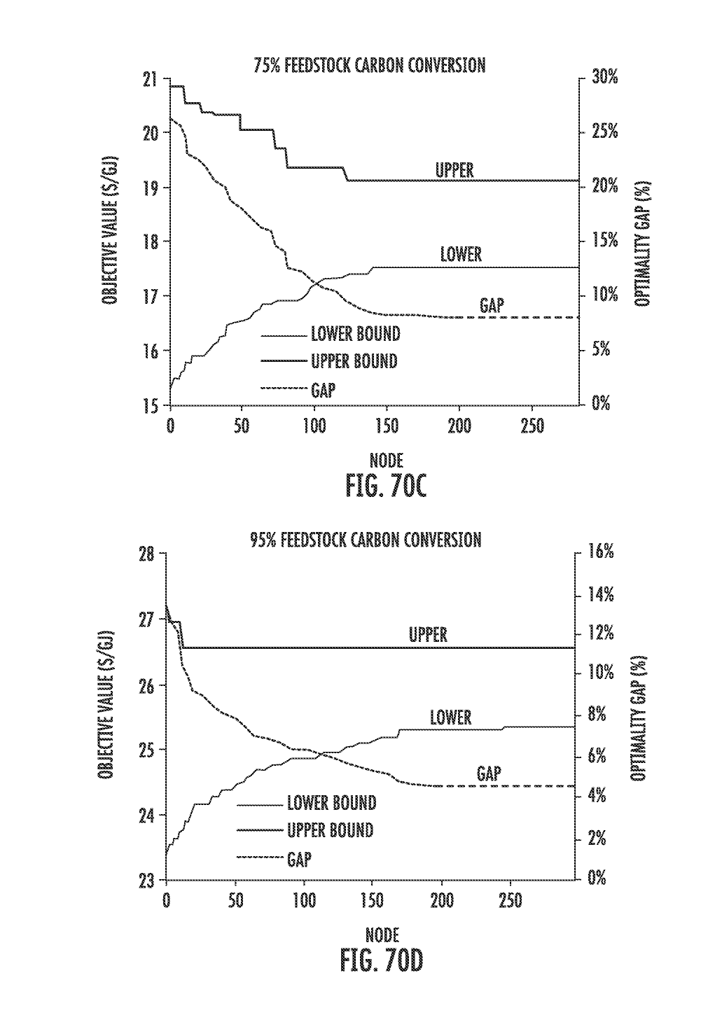

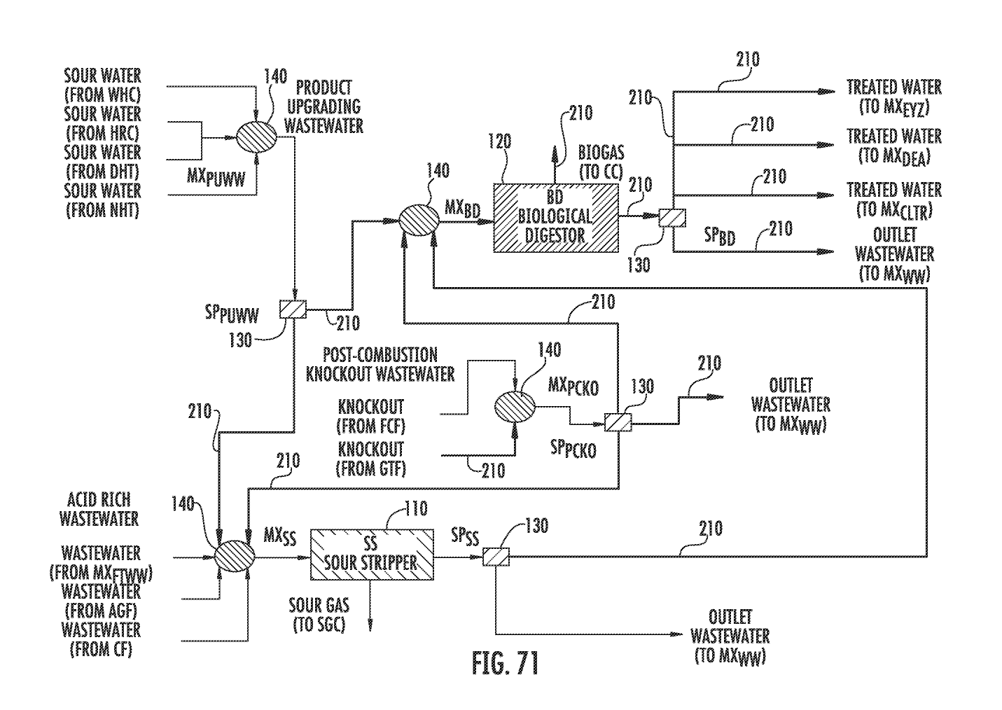

To highlight the change in the lower bound, upper bound, and optimality gap throughout the branch-and-bound tree, the progression of the tree is shown in FIG. 68 for the four small capacity case studies, in FIG. 69 for the four medium case studies, and in FIG. 63 for the large case studies. In each figure, the upper bound (dark) will generally be flat for several nodes and will then experience a drop at a given node. When the upper bound remains flat, it implies that no feasible point was obtained at the node that has a lower objective value than that of the current incumbent solution. If a better feasible solution is found at a node, then the upper bound is updated with this lower objective value, and the curve drops to reflect this change. The lower bound (light), generally increases for each node based upon the partitioning used throughout the tree. However, for each case study, there is a point at which the lower bound does not change as the tree is progressed. At this point, the branch-and-bound tree has been progressed deeply enough where it becomes difficult to partition the search space effectively. The optimality gap (dotted) decreases in accordance with the changes in the lower and upper bounds and generally reaches a threshold value prior to the termination point.

### Example 5.4.2

### Comparative Studies

To benchmark the proposed global optimization method, a comparison of the approach with the deterministic global optimization solvers BARON 9.0.2 and LINDOglobal 6.1.1 was performed using the three 50% conversion case studies. The results are presented in Table 64. Both BARON and LINDOglobal were unable to find a feasible solution (upper bound) to the mathematical model after 100 h of computational time. In addition, the lower bound reported by these two algorithms was smaller than the lower bound reported by the proposed global optimization method for each of the three case studies. The lower bound reported by BARON was 3.4% lower than the lower bound reported by the proposed method for the small case study, 5.7% lower for the medium case study, and 4.5% lower for the large case study. The lower bound for LINDOglobal was 4.7% lower for the small case study, 6.5% lower for the medium case study, and 4.5% lower for the large case study. This implies that the proposed method provided a tighter mathematical guarantee of optimality then either BARON or LINDOglobal was able to do. In addition, the proposed global optimization algorithm was compared to the local solver DICOPT using a multi-start technique. The DICOPT solver was able to find feasible solutions, but could not identify an upper bound that had a lower objective than the upper bound reported by the proposed method.

### Example 5.4.3

### Overall Cost of Liquid Fuels

The upper bound value found at termination of the global optimization algorithm represents the cost of liquid fuels production (in $/GJ) for each case study. This cost is decomposed in Table 65 to highlight the contributions of the feedstocks, investment, sequestration, and byproducts to the final value. The feedstock cost is distributed over the three major carbon based feedstocks (coal, biomass, and natural gas) along with butanes that are needed for isomerization and freshwater that is needed to make-up for losses from the cooling tower and the outlet wastewater. The similarities in the upgrading section for all twelve case studies causes the cost for the butane to remain relatively consistent. Though the freshwater input to the process may vary more widely for each of the twelve case studies, the total cost for the water is minimal when compared to the cost of the remaining feeds. For the biomass feedstock, the contribution to the overall cost generally decreases with increasing carbon conversion rate for each plant size. This is a result of the reduction in the amount of carbon vented from the process as the feedstock-carbon conversion rate increases. As each plant is forced to maintain a 50% reduction in greenhouse gas emissions from petroleum based processes, an increase in the amount of carbon vented will require an increase in biomass input to the system to properly balance the CO₂ lifecycle. For the coal and natural gas feedstocks, the contribution toward the overall cost also decreases as the feedstock-carbon conversion rate increases. This is expected since higher feedstock-carbon conversion implies that a smaller amount of feedstock is needed to produce a similar amount of liquid fuels. Note that the general trends for the coal and natural gas feedstocks are not observed when increasing the conversion rate from 50% to 75%. For each of the three capacities, the biomass cost significantly drops while the cost for coal and natural gas increases slightly. The increase from 50% to 75% conversion marks a transition for the CBGTL process that suggests it is not economically feasible to input additional biomass to balance the CO₂ that is vented from the system. Thus, the CO₂ venting is minimized, the biomass input flow rate is reduced, and the coal and natural gas feedstocks are increased to provide the additional input carbon. The propane produced from the process is a byproduct of the upgrading section (Baliban et al., 2010, 2011; Bechtel, 1992) and therefore is relatively consistent across all twelve case studies.

CO₂ sequestration is only utilized in four of the case studies (S-25, S-75, M-75, and L-75). For most of the 25% and 50% feedstock carbon conversion cases, the results of the mathematical model show that it is more economical to purchase additional biomass and vent the CO₂ rather than sequester the CO₂ and purchase cheaper, fossil-fuel feedstocks. In these cases, the CO₂ that is vented largely comes from generation of the electricity via an air-blown gas turbine. The combination of CO₂ and N₂ in the turbine effluent makes CO₂ capture and sequestration an economically unfavorable alternative to simply venting the CO₂ and using more biomass as a feed. For the 95% conversion case studies, CO₂ sequestration is also not utilized in the final process topology since most of the CO₂ is reacted with H₂ to form CO via the reverse water-gas-shift reaction. The 75% carbon conversion studies all show that CO₂ sequestration should be utilized to handle some of the unreacted carbon. Once a certain feedstock-carbon conversion threshold is passed, then some of the CO₂ must be reacted with H₂ to obtain the conversion rate necessary. This requires the use of electrolyzers which input electricity to produce the necessary H₂, the result of which can be seen as a positive contribution of the electricity to the overall cost. Some of this electricity may be recovered through the use of a gas turbine, but the recovery of CO₂ from the turbine effluent will be limited due to the N₂ present in the gas turbine inlet air. The resulting CO₂ within the process is partially sequestered because this option is economically favorable compared to the high electricity cost of the electrolyzers. Note that the 95% conversion cases rely heavily on the electrolyzers and therefore require a significant contribution from non-carbon based electricity.

The final contribution to the overall cost comes from the investment of the process units. For each plant capacity, the investment cost is highest for the 25% and 95% conversion cases. The 25% conversion cases produce a significant amount of byproduct electricity (high negative values in Table 65), which require higher feedstock inputs and larger working capacities across all units throughout the process topology. As the amount of feedstock-carbon conversion increases, then a smaller amount of the synthesis gas is directed to the gas turbines, resulting in a decrease in the output electricity and the investment cost. The decrease in working flow rates throughout the system also contributes a smaller amount of waste-heat than the 25% conversion case, which reduces the electricity output and the investment cost of the heat and power recovery system. The 75% conversion cases also have a decrease for the medium and large capacity plants, but there is an increase for the small capacity plant. In this instance, the decrease in investment cost from smaller working flow rates is partially offset by the high investment cost of the electrolyzer. This fact is further emphasized in the 95% conversion cases, as the investment cost is higher than any other conversion rate. Note that this trend is solely an effect of the electrolyzer investment cost, and if this unit investment cost was reduced, the 95% conversion cases would likely have the lowest overall investment cost.

TABLE 64
Comparative studies.

| | | | | |
|---|---|---|---|---|
| Bound | Proposed approach | BARON | LINDOGlobal | DICOPT |

Small plant capacity (10 TBD)

| | | | | |
|---|---|---|---|---|
| UB | 13.01 | — | — | 13.71 |
| LB | 12.42 | 12.01 | 11.86 | N/A |

Medium plant capacity (50 TBD)

| | | | | |
|---|---|---|---|---|
| UB | 12.85 | — | — | 12.99 |
| LB | 11.75 | 11.12 | 11.03 | N/A |

Large plant capacity (200 TBD)

| | | | | |
|---|---|---|---|---|
| UB | 11.97 | — | — | 12.77 |
| LB | 11.00 | 10.53 | 10.51 | N/A |

The best upper bound (UB) and lower bound (LB) are presented for each algorithm when compared for each 50% conversion case study. The computational time alloted for each algorithm was capped at 100 CPU hours. A “-” symbol indicates that an algorithm was unable to obtain a feasible solution after the computational time was exhausted. The “N/A” for DICOPT is present because this algorithm will not provide information on the lower bound.

The resulting components of the overall cost combine to provide a range of $12.54/GJ-$29.54/GJ for the small case studies, $12.03/GJ-$28.56/GJ for the medium case studies, and $11.32/GJ-$26.49/GJ for the large case studies. Using the refiner's margin for gasoline, diesel, and kerosene (Baliban et al., 2011; Kreutz et al., 2008), the corresponding price of crude oil that will be equivalent to the cost of liquid fuels is calculated and displayed in Table 65. This break-even oil price (BEOP) can be thought of as the price of crude oil at which the CBGTL process becomes economically competitive with petroleum based processes. This cost ranges from $58.68/bbl to $155.56/bbl for the small facilities, $55.77/bbl $149.98/bbl for the medium facilities, and $51.73/bbl-$138.18/bbl for the large facilities. As an illustrative example, the results for 50% conversion of carbon are shown in boldface in Table 65. The cost ranges from $61.36/bbl for small plants, $60.45/bbl for medium plants, and $55.43/bbl for large plants.

The 25% and 50% conversion cases have BEOPs that are at the low end of the range, and the difference in cost between the 50%, 75%, and 95% cases is much higher than between 25% and 50%. This is a direct effect of the cost of electricity and investment needed to power the electrolyzer unit that converts some CO₂ into CO via the reverse water-gas-shift reaction. In this study, the electricity price is set to $0.07/kWhr and the electolyzer base investment cost is $1000/kW (National Research Council, 2004). Though a reduction in investment cost can help reduce the overall costs for the 75% and 95% cases, the bulk of the price will be from electricity. If cheaper means of electricity production are obtained, then the BEOP for the 75% and 95% cases will decrease dramatically.

### Example 5.4.4

### Optimal Process Topologies

The information detailing the optimal process topologies for all case studies is shown in Table 66. Three possible temperature options were used for the biomass gasifier (900° C., 1000° C., 1100° C.), the coal gasifier (1100° C., 1200° C., 1300° C.), the auto-thermal reactor (700° C., 800° C., 900° C.), and the reverse water gas-shift unit (700° C., 800° C., 900° C.). For the biomass gasifier, the 1100° C. unit is selected for the 25% conversion rate across all three capacities. For the remaining nine case studies, the 900° C. unit is selected. For the coal gasifier, the 1200° C. unit was selected for four of the case studies and the 1300° C. unit was selected for the remaining eight case studies. Note that both the biomass and coal gasifiers for all twelve case studies were solid/vapor fueled units which employed a vapor phase recycle stream as a fuel input along with the solid coal or biomass.

The reverse water-gas-shift unit was selected for all 25% and 50% conversion case studies with an operating temperature of 700° C. or 900° C. For the 75% and 95% case studies, no dedicated reverse water-gas-shift unit was selected in the optimal topology because the consumption of the CO₂ occurred within the iron-based Fischer-Tropsch (FT) units or the gasifiers. In fact, an iron-based low-temperature FT unit was selected for all of the twelve case studies, and an iron-based high-temperature FT unit was used in seven of the studies. Each of these iron-based units can take advantage of the exothermic FT reaction to provide heat for the endothermic reverse water-gas-shift reaction (Baliban et al., 2011, 2012). The high-temperature FT units for the remaining five case studies utilize a cobalt-based catalyst that has a minimum amount of CO₂ input and does not facilitate the water-gas-shift reaction. The auto-thermal reactor temperature was selected to be 800° C. for four of the case studies and 900° C. for the remaining eight studies (see Table 66).

TABLE 65
Overall cost results for the twelve case studies.

| Case | Contribution to cost ($/GJ) | | | | | | | | | | |
|---|---|---|---|---|---|---|---|---|---|---|---|
| study | Coal | Biomass | Nat. gas | Butane | Water | CO₂ seq. | Inv. | Elec. | Propane | Total ($/GJ) | Total ($/bbl) |
| S-25 | 4.93 | 7.65 | 2.21 | 0.52 | 0.02 | 0.27 | 5.52 | −8.07 | −0.51 | 12.54 | 58.68 |
| S-50 | 2.41 | 7.68 | 1.12 | 0.51 | 0.02 | 0.00 | 4.73 | −2.85 | −0.60 | 13.01 | 61.36 |
| S-75 | 2.71 | 2.35 | 1.94 | 0.58 | 0.02 | 0.54 | 5.01 | 9.46 | −0.58 | 22.03 | 112.76 |
| S-95 | 1.84 | 2.58 | 1.32 | 0.57 | 0.03 | 0.00 | 5.97 | 17.79 | −0.56 | 29.54 | 155.56 |
| M-25 | 2.98 | 12.23 | 2.13 | 0.57 | 0.04 | 0.00 | 3.25 | −8.60 | −0.56 | 12.03 | 55.77 |
| M-50 | 2.72 | 7.60 | 1.22 | 0.51 | 0.02 | 0.00 | 2.88 | −1.60 | −0.50 | 12.85 | 60.45 |
| M-75 | 2.98 | 2.44 | 1.33 | 0.59 | 0.02 | 0.39 | 2.39 | 11.88 | −0.58 | 21.43 | 109.34 |
| M-95 | 2.04 | 2.57 | 0.92 | 0.62 | 0.03 | 0.00 | 3.54 | 19.45 | −0.61 | 28.56 | 149.98 |
| L-25 | 3.03 | 12.70 | 2.17 | 0.61 | 0.03 | 0.00 | 2.05 | −8.69 | −0.59 | 11.32 | 51.73 |
| L-50 | 2.43 | 7.65 | 1.74 | 0.57 | 0.03 | 0.00 | 1.75 | −1.62 | −0.58 | 11.97 | 55.43 |
| L-75 | 2.63 | 2.47 | 1.89 | 0.50 | 0.03 | 0.48 | 1.53 | 10.07 | −0.50 | 19.11 | 96.12 |
| L-95 | 1.83 | 2.56 | 1.31 | 0.55 | 0.03 | 0.00 | 2.13 | 18.63 | −0.54 | 26.49 | 138.18 |

The small (S), medium (M), and large (L) case studies are each labeled with the percentage of feedstock carbon that must go to liquid fuels. The contribution to the total costs (in $/GJ) come from coal, biomass, natural gas (Nat. Gas.), butanes, water, CO2 sequestration (CO2. Seq.), and the investment (Inv.). Propane is always sold as a byproduct while electricity (Elec.) may be sold as a byproduct (negative value) or obtained from a non-carbon based source (positive value). The results for 50% conversion of feedstock carbon are shown in boldface.

TABLE 66
Topological information for the optimal solutions for the twelve case studies.

| Case study | S-25 | S-50 | S-75 | S-95 | M-25 | M-50 | M-75 | M-95 | L-25 | L-50 | L-75 | L-95 |
|---|---|---|---|---|---|---|---|---|---|---|---|---|
| BGS Temp. | 1100 | 900 | 900 | 900 | 1100 | 900 | 900 | 900 | 1100 | 900 | 900 | 900 |
| BGS Type | S/V | S/V | S/V | S/V | S/V | S/V | S/V | S/V | S/V | S/V | S/V | S/V |
| CGS Temp. | 1200 | 1300 | 1300 | 1300 | 1300 | 1200 | 1200 | 1300 | 1300 | 1200 | 1300 | 1300 |
| CGS Type | S/V | S/V | S/V | S/V | S/V | S/V | S/V | S/V | S/V | S/V | S/V | S/V |
| RGS Temp. | 700 | 700 | — | — | 700 | 900 | — | — | 700 | 700 | — | — |
| ATR temp. | 800 | 800 | 900 | 900 | 800 | 800 | 900 | 900 | 800 | 900 | 900 | 900 |
| EYZ Usage | N | N | Y | Y | N | N | Y | Y | N | N | Y | Y |
| CO2SEQ Usage | Y | N | Y | N | N | N | Y | N | N | N | Y | N |
| GT Usage | Y | Y | Y | N | Y | Y | Y | N | Y | Y | Y | N |
| LTFT Type | Iron | Iron | Iron | Iron | Iron | Iron | Iron | Iron | Iron | Iron | Iron | Iron |
| HTFT Type | Cobalt | Cobalt | Iron | Iron | Cobalt | Cobalt | Iron | Iron | Cobalt | Iron | Iron | Iron |

Specifically listed is the operating temperature of the biomass gasifier (BGS), the coal gasifier (CGS), the auto-thermal reactor (ATR), and the reverse water-gas-shift unit (RGS). The gasifiers are also labeled as either solid/vapor (S/V) or solid (S) fueled, implying the presence or absence of vapor-phase recycle process streams. The presence of an electrolyzer (EYZ), a CO2 sequestration system (CO2SEQ), or a gas turbine (GT) is noted using yes (Y) or no (N). The low and high-temperature Fischer-Tropsch units (LTFT and HTFT) are designated as either iron-based or cobalt-based units. The results for 50% conversion of feedstock carbon are shown in boldface.

The 25% and 50% conversion case studies do not use an electrolyzer, but choose to mostly vent the generated CO₂. In the S-25 case study, a small amount of the CO₂ is sequestered (see Table 67). All of the 75% and 95% studies must use an electrolyzer to convert some of the CO₂ into liquid fuels, though only the 75% case studies utilize sequestration to remove the remaining CO₂. The 25%, 50%, and 75% conversion studies all have a gas turbine installed to help recover some of the electricity needed for the process and potentially sell extra electricity as a byproduct. The gas turbine is not selected for the 95% case study due to the high cost of CO₂ that must be recovered from the turbine outlet.

The 50% conversion cases are listed in boldface in Table 67. For each of these cases, the biomass gasifiers were solid/vapor fueled units operating at 900° C. The coal gasifiers were solid/vapor fueled units operating at 1200° C. for the large and medium case studies and 1300° C. for the small case study. The reverse water-gas-shift unit operates at 700° C. for the small and large case studies and 900° C. for the medium case study. The auto-thermal reactor operates at 800° C. for the small and medium case studies and 900° C. for the large case study. The low-temperature FT reactor used was iron-based for all three studies and the high-temperature FT reactor was cobalt-based for the small and medium case studies and iron-based for the large case study.

### Example 5.4.5

### Overall Process Material Balances

The overall carbon balance for the CBGTL processes is shown in Table 67 and highlights the eight major points where carbon is either input or output from the system. The 50% conversion case studies are highlighted in the table using boldface. Carbon that is input to the system via air is neglected due to the low flow rate relative to the other eight points. The coal, biomass, and natural gas inputs generally supply over 99% of the input carbon to the system while the balance is supplied by the butane input to the upgrading units. Note that the input carbon flow rate for the feedstocks changes similarly to the changes seen with the feedstock cost contributions in Table 65. That is, generally, the carbon input for each feedstock decreases as the carbon conversion rate increases. The strong decrease in the biomass cost from 50% to 75% conversion seen in Table 65 is supported by the decrease in the carbon input shown in Table 67 for these case studies. Additionally, it is evident that the carbon vented from the system decreases by over 95% for each capacity when moving from a 50% to a 75% conversion of feedstock carbon. In the 75% conversion cases, the unreacted carbon is sequestered as this proves to be the economically preferable option to increasing the biomass feedstock.

The output amount of carbon in the total product is constant for each plant capacity, which is consistent with the constant production capacity that is required for each feedstock-conversion rate. The amount of carbon leaving as propane is around 3% of that leaving as gasoline, kerosene, and diesel. For eight of the twelve case studies, the remaining carbon exits as CO₂ during venting, as this is the economically preferable option. For the S-25 case study, a small amount of the carbon is sequestered, and for the three 75% conversion case studies, a majority of the carbon is sequestered as CO₂.

TABLE 67
Overall carbon balance for the CBGTL process.

| Case | Carbon input (kmol/s) | | | | Carbon output (kmol/s) | | | | Conversion |
|---|---|---|---|---|---|---|---|---|---|
| study | Coal | Biomass | Nat. gas | Butane | Product | Propane | Vent CO₂ | Seq. CO₂ | (%) |
| S-25 | 1.37 | 1.18 | 0.34 | 0.02 | 1.05 | 0.03 | 1.07 | 0.76 | 37 |
| S-50 | 0.72 | 1.19 | 0.23 | 0.02 | 1.05 | 0.03 | 1.08 | 0.00 | 50 |
| S-75 | 0.75 | 0.36 | 0.30 | 0.02 | 1.05 | 0.03 | 0.01 | 0.35 | 75 |
| S-95 | 0.51 | 0.40 | 0.20 | 0.02 | 1.05 | 0.03 | 0.06 | 0.00 | 95 |
| M-25 | 4.14 | 9.45 | 1.66 | 0.12 | 5.23 | 0.15 | 0.99 | 0.00 | 35 |
| M-50 | 3.79 | 5.87 | 0.95 | 0.11 | 5.23 | 0.13 | 5.36 | 0.00 | 50 |
| M-75 | 4.15 | 1.88 | 1.04 | 0.12 | 5.23 | 0.16 | 0.15 | 1.65 | 75 |
| M-95 | 2.84 | 1.99 | 0.71 | 0.13 | 5.23 | 0.16 | 0.28 | 0.00 | 95 |
| L-25 | 16.88 | 39.28 | 6.75 | 0.51 | 20.92 | 0.64 | 41.85 | 0.00 | 34 |
| L-50 | 13.55 | 23.64 | 5.42 | 0.48 | 20.92 | 0.62 | 21.54 | 0.00 | 50 |
| L-75 | 14.68 | 7.65 | 5.87 | 0.42 | 20.92 | 0.54 | 0.83 | 6.32 | 75 |
| L-95 | 10.19 | 7.91 | 4.08 | 0.46 | 20.92 | 0.58 | 1.13 | 0.00 | 95 |

Carbon is input to the process via coal, biomass, natural gas, or butanes and exits the process as liquid product, propane byproduct, vented CO₂, or sequestered (seq.) CO₂. The small amount of CO₂ input to the system in the purified oxygen stream (<0.01%) is neglected. The results for the 50% conversion of feedstock carbon are shown in boldface.

TABLE 68
Overall material and energy balances for the CBGTL process.

| | | | | | | | | | |
|---|---|---|---|---|---|---|---|---|---|
| | Coa1 | Biomass | Nat. gas | Butane | Freshwater | Wastewater | Propane | CO₂ vent | CO₂ seq. |
| Case study | (DT/TB) | (DT/TB) | (MSCF/TB) | (bbl/TB) | (bbl/bbl) | (bbl/bbl) | (bbl/TB) | (kg/bbl) | (kg/bbl) |

Material balances

| S-25 | 196.33 | 300.57 | 2.18 | 115.10 | 2.19 | 0.49 | 139.11 | 408.74 | 190.51 |
|---|---|---|---|---|---|---|---|---|---|
| S-50 | 103.59 | 301.56 | 1.46 | 130.60 | 2.61 | 0.40 | 164.28 | 410.67 | 0.00 |
| S-75 | 107.92 | 92.35 | 1.92 | 128.39 | 2.52 | 0.26 | 158.32 | 3.80 | 133.09 |
| S-95 | 73.28 | 101.51 | 1.30 | 126.17 | 2.79 | 0.21 | 152.48 | 21.61 | 0.00 |
| M-25 | 118.53 | 480.33 | 2.11 | 126.17 | 2.85 | 0.06 | 152.48 | 759.84 | 0.00 |
| M-50 | 108.42 | 298.43 | 1.21 | 112.89 | 2.07 | 0.25 | 136.43 | 407.82 | 0.00 |
| M-75 | 118.58 | 95.74 | 1.32 | 130.60 | 2.32 | 0.22 | 159.44 | 11.15 | 125.48 |
| M-95 | 81.33 | 101.10 | 0.91 | 137.24 | 2.39 | 0.04 | 165.86 | 21.57 | 0.00 |
| L-25 | 120.65 | 499.16 | 2.15 | 135.03 | 2.26 | 0.39 | 161.52 | 795.70 | 0.00 |
| L-50 | 96.84 | 300.39 | 1.72 | 126.17 | 2.83 | 0.08 | 158.71 | 409.53 | 0.00 |
| L-75 | 104.92 | 97.17 | 1.87 | 110.68 | 2.37 | 0.34 | 136.49 | 15.84 | 120.16 |
| L-95 | 72.85 | 100.48 | 1.30 | 121.74 | 2.85 | 0.21 | 148.63 | 21.51 | 0.00 |
| Case study | Coal | Biomass | Nat. gas | Butane | Electricity | Product | Propane | Efficiency | |

Energy balances (in MW LHV)

| | | | | | | | | |
|---|---|---|---|---|---|---|---|---|
| S-25 | 701 | 548 | 249 | 80 | −274 | 659 | 97 | 65.3 |
| S-50 | 370 | 550 | 166 | 91 | −97 | 659 | 114 | 73.9 |
| S-75 | 385 | 168 | 219 | 89 | 321 | 659 | 110 | 65.1 |
| S-95 | 262 | 185 | 148 | 88 | 603 | 659 | 106 | 59.5 |
| M-25 | 2116 | 4378 | 1201 | 438 | −1458 | 3297 | 529 | 65.0 |
| M-50 | 1936 | 2720 | 686 | 392 | −271 | 3297 | 474 | 70.5 |
| M-75 | 2117 | 873 | 751 | 453 | 2014 | 3297 | 554 | 62.0 |
| M-95 | 1452 | 921 | 515 | 477 | 3297 | 3297 | 576 | 58.1 |
| L-25 | 8617 | 18,197 | 4888 | 1875 | −5894 | 13,190 | 2243 | 63.5 |
| L-50 | 6917 | 10,951 | 3924 | 1752 | −1099 | 13,190 | 2204 | 70.1 |
| L-75 | 7494 | 3542 | 4251 | 1537 | 6833 | 13,190 | 1896 | 63.8 |
| L-95 | 5203 | 3663 | 2951 | 1691 | 12,635 | 13,190 | 2064 | 58.3 |

bbl, barrels; TB, thousand barrels; DT, dry tons; MSCF, million standard cubic feet; LHV, lower heating value.

All material balances are normalized with respect to the volume of products produced. Negative electricity values in the energy balance represent outlet energy, and are counted as products for the efficiency rate and positive values are added as input to the CBGTL process. The results for 50% conversion of feedstock carbon are shown in boldface.

For each of the case studies, the carbon conversion rate was set as a lower bound for the mathematical model. Thus, the conversion of carbon in the four feedstocks to the final liquid products (i.e., gasoline, diesel, and kerosene) must be at least as large as the set conversion rate. For nine of the case studies, the final conversion rate was exactly equal to the rate specified to the model. The results of the mathematical model suggest that it is more economically favorable to vent or sequester the CO₂ rather than react it with H₂ to produce additional liquid fuels. This is consistent with the high costs associated with the electrolyzer to produce the necessary H₂ for reaction. For the 25% conversion case studies, Table 67 shows that the mathematical model chooses conversion rates between 34% and 37% for the optimal process design. In these instances, it is more beneficial to produce additional liquid fuels as opposed to electricity via the gas and steam turbines.

The overall material and energy balances for each case study are shown in Table 68. Each component in the material balances is normalized with respect to the amount of liquid products produced. The coal and biomass flow rates are based on dry tons while the natural gas is shown in million standard cubic feet. The change in feedstock flow rate with increasing carbon conversion rate is consistent with the results shown above, but the normalization of the flow rates shows that the feedstock flow rates have similar values for the same conversion rate across all three plant capacities. This is in agreement with the cost data shown in Table 65 and the similar topological data shown in Table 66. The remaining feedstocks, butane and freshwater, along with the outlet wastewater and byproduct propane vary over a small range. The CO₂ that is vented or sequestered from the process decreases as expected with increasing conversion rate and ranges over all twelve studies from 3.80 kg/bbl for the S-75 case study to a total of 795.70 kg/bbl for both venting and sequestration for the L-25 case study.

As an illustrative example, the 50% conversion case studies shown in boldface in Table 68. The amount of coal feedstock used for the system is 103.59 dry tons/thousand barrels (DT/TB) for the small capacity, 108.42 DT/TB for the medium capacity, and 96.84 DT/TB for the large capacity. The biomass input is 301.56 DT/TB for the small capacity, 298.43 DT/TB for the medium capacity, and 300.39 DT/DB for the large capacity. The natural gas input is 1.46 million standard cubic feet/thousand barrels (MSCF/TB) for the small capacity, 1.21 MSCF/TB for the medium capacity, and 1.72 MSCF/TB for the large capacity. The freshwater required for the case studies is 2.61 barrels/barrel of product (bbl/bbl) for the small system, 2.07 bbl/bbl for the medium case, and 2.83 bbl/bbl for the large case while the outlet wastewater is 0.40 bbl/bbl for the small case, 0.25 bbl/bbl for the medium case, and 0.08 bbl/bbl for the large case. The CO₂ produced from these three case studies is vented at rates of 410.67 kg/bbl for the small case, 407.62 kg/bbl for the medium case, and 409.53 kg/bbl for the large case. CO₂ sequestration is not utilized for all three studies.

The energy balances from the process are listed in MW in Table 68. The small capacity plant is designed to produce 659 MW of fuels on a lower heating value basis, the medium plant is designed for 3297 MW, and the large plant for 13,190 MW. The amount of energy needed to produce the liquid fuels ranges from 765 MW to 1030 MW for the small plants, from 3851 MW to 5284 MW for the medium plants, and from 15,086 MW to 21,327 MW for the large plants. The efficiency of the system is calculated by dividing the total energy output (i.e., via products, propane, or electricity) by the total energy input (i.e., via coal, biomass, natural gas, butane, or electricity). If electricity is output from the system, the value is listed as negative in Table 68 and the magnitude of the energy value is added to the total output. If the value is positive, then this energy is added to the total input to the system. The efficiency of the twelve studies ranges from 58.1% for the M-95 process to 73.9% for the S-50 process.

As an illustrative example, the 50% case studies are shown in boldface in Table 68. Each of these studies will output electricity as a byproduct, so the energy required for fuels production is the sum of the lower heating values of the feedstocks. In general, the biomass is the largest contributor to the energy input at 550 MW for the small study, 2720 MW for the medium study, and 10,951 MW for the large study. Coal is the next highest contributer with 370 MW needed for the small study, 1936 MW needed for the large study, and 6917 MW needed for the large study. The balance of the energy input is split between natural gas and butanes. The energy output from the process is in the form of liquid product (i.e., gasoline, diesel, and kerosene), liquid byproduct (propane), or electricity. The largest output of energy is the liquid product, the second highest is the propane, and the last is the electricity. The efficiencies for the 50% conversion cases represent the highest for a given capacity over all conversion rates. The small plant has the largest overall efficiency of 73.9%, while the medium and large plants have efficiencies of 70.5% and 70.1%, respectively.

### Example 5.5

### Conclusions

A novel global optimization framework has been proposed to address the large-scale coal, biomass, and natural gas to liquids (CBGTL) process synthesis mathematical model with simultaneous heat, power, and water integration. Using piecewise linear underestimators with a logarithmic partitioning scheme for the bilinear terms and piecewise linear underestimators with a linear partitioning scheme for the concave cost functions, twelve case studies for the CBGTL model have been optimized to within a 3.22-8.56% optimality gap after 100 CPU hours. The case studies arise from four distinct carbon-conversion rates for a small (10 TBD), medium (50 TBD), and large (200 TBD) plant capacities, all of which must have a 50% reduction in greenhouse gases from petroleum-based processes. The proposed framework shows that the break-even oil price for liquid fuels production ranges from $58.68/bbl to $155.56/bbl for the small case studies, $55.77/bbl-$149.98/bbl for the medium case studies, and $51.73/bbl-$138.18/bbl for the large case studies. For a feedstock carbon conversion rate of 50%, the cost is $61.36/bbl for the small study, $60.45/bbl for the medium study, and $55.43/bbl for the large study. Each of the three 50% conversion case studies did not utilize CO2 sequestration to reduce GHG emissions, but instead incorporated a larger amount of biomass feedstock into the refinery. While the biomass feedstock represented a large fraction (over 60%) of the cost of each of the 50% conversion case studies, this option will be favorable to CO₂ sequestration because of the reduction in byproduct electricity that would occur with the latter choice.

When the conversion rate of feedstock carbon is analyzed parametrically using the proposed optimization framework, a clear trend in the increase of the liquid fuels cost is observed. Utilization of domestic resources to maximum capability is a high concern for energy sustainability, but there is a clear point where the cost of feedstock conversion is not justified due to high costs of electricity and key process units. The proposed framework is able to systematically identify the point where the conversion rate of the carbon in the feedstock may be increased without affecting the end consumer of liquid fuels. The proposed framework represents a rigorous methodology for systematically analyzing the economic and environmental tradeoffs associated with the construction of hybrid coal, biomass, and natural gas facilities and can ensure a process design will have a cost of fuels production that is at the global optimum of the highly nonconvex search space. The global optimization framework is instrumental not only in providing a tight lower bound on the optimal solution, but also in identifying process topologies that were not obtained through local optimization only. That is, the final process topologies selected from the proposed framework were different than the topology selected at the root node (i.e., through local optimization). This implies that the proposed method is critical for both reducing the cost of the overall refinery and uncovering process topologies that may be difficult to obtain based on their location in the search space.

### Example 6

### Wastewater Network

To accompany the above process superstructure, a complete water treatment network (FIGS. 71 and 72) is postulated that will treat and recycle (a) wastewater from various process units, (b) blowdown from the cooling tower, (c) blowdown from the boilers, and (d) input freshwater. The treatment units include (i) a sour stripper and (ii) a biological digester unit to remove acids and hydrocarbon components entrained in the water stream and (iii) a reverse osmosis system to remove suspended and dissolved solids from blowdown streams. Clean output of the network includes (i) process water to the electrolyzers, (ii) steam to the gasifiers, autothermal reactor, and water-gas-shift reactor, and (iii) discharged wastewater to the environment. The biogas from the biological digester and the sour gas from the sour stripper will be recycled back to the CBGTL process. All separated solids from the reverse osmosis system will be discharged as solid waste. The general superstructure allows for single or multiple water sources with or without contaminants, water-using units, and wastewater treatment units, with all feasible stream connections considered between these units.

### Example 6.1

### Process Wastewater Superstructure

In the previous model, all process wastewater was treated in a sour stripper (SS) that removed all entrained vapor from the inlet water. The acid-rich vapors were recycled back to a sour gas compressor while the treated water was split and sent to either the deaerator for steam generation or the electrolyzer for hydrogen/oxygen production. Although the sour stripper can remove a large portion of the entrained hydrocarbons, CO₂, and H₂S from the inlet feed, there is a limitation on the amount of NH₃ that can be recovered. To comply with environmental regulations, the sour stripper effluent must either be diluted with freshwater or treated in a secondary processing unit.

Biological digestion via anaerobic and aerobic digestion can reduce the hydrocarbon and acid gases in the inlet stream by converting them to biogas (i.e., CH₄, CO₂, H₂, NH₃, and Ar), which can be sent back to the Claus combuster along with the compressed and heated sour gas stream from sour stripper. Thus, the biological digestor (BD) unit can act as a primary processing unit for streams that have low enough contaminant level for the operation of the unit (i.e., sour water from the upgrading section, post-combustion knockout wastewater) and as a secondary processing unit for the sour stripper effluent. High conversion to biogas is achieved and the effluent water can be readily used in various water-using units or disposed as wastewater to the environment.

FIG. 71 illustrates the outline of the process wastewater stream superstructure. Fixed process units are represented by 110, variable process units are illustrated by 120, variable process streams are represented by 210 and all other process streams are fixed unless otherwise indicated. Wastewater from the CBGTL process includes knockout water from the acid gas flash unit (AGF), the Claus flash unit (CF), the aqueous effluent from Fischer-Tropsch hydrocarbon production (MXFTWW), and the sour water effluent from the product upgrading units. Based on previous experience with the process, the concentration of acid gases present in the acid gas flash knockout (AGF) and the Clash flash knockout (CF) may be too high for operation of the biological digestor (BD). Therefore, these two streams are passed directly to the sour stripper (SS). Additionally, the concentration of dissolved hydrocarbons in the FT wastewater (MXFTWW) exceeds the maximum limit of contaminants for the (BD) unit, so this stream is also passed directly to the (SS) unit. Direction of the above streams to the (SS) unit implies that this unit will always be present in any selected CBGTL process topology. The effluent of the (SS) unit may be split (SPSS) and sent to the (BD) unit to remove any remaining entrained NH₃ in the stream or to the outlet mixer (MXWW). Treated water from the (BD) unit is split (SPBD) and recycled as treated water to the electrolyzer, the cooling tower, or the deaerator, or is discharged as outlet wastewater.

An additional source of process wastewater comes from the post-combustion knockout units. This includes the fuel combustor flash (FCF) and gas turbine flash unit (GTF) which are combined (MXPCKO) before being split (SPPCKO) to either to (SS) unit, the (BD) unit, or directly to the outlet wastewater mixer (OUTWW). Note that this is the only process stream that is directly allowed to go to the outlet due to the low level of contaminants in the stream. The remainder of wastewater from CBGTL process units is derived from the hydrocarbon recovery unit (HRC), wax hydrocracker (WHC), distillate hydrotreater (DHT), and naphtha hydrotreater (NHT) and is combined as one generic output stream (MXPUWW) before entering the water treatment section. The process upgrading unit wastewater streams are merged into a singular wastewater stream based on the modeling in Baliban et al. (2011), which is incorporated herein by reference as if fully set forth. Due to the rigorous gas cleaning upstream of the FT units, very little sulfur will exist in the product upgrading wastewater. Composition of phenols and other water-soluble hydrocarbons have also been reduced by the series of separation units for the FT effluents. Thus, further cleaning of this stream can take place in either the (SS) unit or the (BD) unit.

### Example 6.2

### Utility Wastewater Superstructure

The superstructure for the wastewater generated by the utility units is shown in FIG. 72. The cooling tower (CLTR) provides the cooling requirement for the CBGTL process (COOL-P) by heating process water from 25° C. to 55° C. A loss fraction due to drift and evaporation will occur in this unit, which is accounted for in the unit's material and energy balance. The circulating fluid will begin to accumulate salts and dissolved solids which can foul the heat exchangers and cause inefficient heat transfer. A blowdown stream must be incorporated with a flow rate set by the cycles of concentration typically used by the cooling tower. Steam is generated within the heat engine boilers inside the heat and power system (HEP) and within the process water boiler (XPWB) for use with the gasifiers (BGS, BRGS, CGS, and CRGS), the auto-thermal reactor (ATR), or the water-gas-shift reactor (WGS). Each of these steam-using units is present in the overall CBGTL superstructure and must have their water requirement satisfied by the utility wastewater superstructure to guarantee water integration. Blowdown streams must also be incorporated within the steam cycle to prevent the build-up of solids and hinder heat transfer. To remove a majority of the solids present in these blowdown streams, a reverse osmosis (RO) unit is used. The (RO) unit may collect (MXRO) a fraction of the cooling tower blowdown (SPCLTR) or the combined blowdown from all process boilers (SPBLR). The effluent of the (RO) unit is split (SPRO) and can be recycled back to the (RO) inlet or sent to the deaerator (DEA), the cooling tower (MXCLTR), or output from the system (OUTWW).

FIG. 72 shows the freshwater input and all outputs from the water treatment network. Input freshwater (INH2O) is assumed to contain no impurities and can either be split (SPH2O) to the outlet to reduce contamination levels, to the electrolyzer mixer (MXEYZ), to the deaerator mixer (MXDEA), or to the cooling tower mixer (MXCLTR). Treated water from the biological digestor (SPBD) can be mixed with the input freshwater before being directed to the electrolyzer (EYZ). Process steam is generated by splitting the output of the deaerator (SPDEA) and directing a cut to the economizer (XPWE) before being boiled (XPWB). The resulting steam is then split (SPSTM) to various process units. The combined output wastewater from various sections of the treatment process is mixed (MXWW) before being output to the environment (OUTWW).

### Example 6.3

### Mathematical Model for Process Synthesis with Simultaneous Heat, Power, and Water Integration

This section will discuss the enhancements to the previous mathematical model for simultaneous process synthesis and heat and power integration, (P), that will incorporate a comprehensive water recovery and treatment section in the CBGTL plant.

### Example 6.3.1

### Heat and Mass Flows

Mass flow for all species is constrained by either a species balance (Eq. (527)/Eq. (528) or an atom balance (Eq. (528)/Eq. (529). The units requiring a species balance, U_{Sp}^{Bal}, will include the mixer units, the splitter units, and the reverse osmosis unit. The units requiring an atom balance, U_{At}^{Bal}, are the sour stripper and biological digestor.

$$\begin{matrix}{\sum }_{( u ′ , u ) ∈ UC} {N}_{u ′ , u , s}^{S} - {\sum }_{( u , r , s ′ ) ∈ {R}^{U}} \frac{{v}_{r , s}}{{v}_{r , s ′}} \cdot {ξ}_{r}^{u} - {\sum }_{( u , u ′ ) ∈ UC} {N}_{u , u ′ , s}^{S} = 0 \\ ∀ s ∈ {S}_{u}^{U} , u ∈ {U}_{Sp}^{Bal} & ( 527 ) \\ {\sum }_{( u ′ , u , s ) ∈ {S}^{UF}} {AR}_{s , a} \cdot {N}_{u ′ , u , s}^{S} - {\sum }_{( u , u ′ , s ) ∈ {S}^{UF}} {AR}_{s , a} \cdot {N}_{u , u ′ , s}^{S} = 0 \\ ∀ a ∈ {A}_{u}^{U} , u ∈ {U}_{At}^{Bal} & ( 528 )\end{matrix}$$

Heat balances across every unit are maintained using Eq. (529). The relevant terms include the input and output stream enthalpies (H), the heat transferred to/from the unit (Q), the heat lost from the unit (Q^{L}), and the work done by the unit (W). Note that Eq. (529) is a general equation for the entire CBGTL refinery, and some of the terms are not needed for each unit. Specifically, the heat loss across all units in the wastewater network is negligible (Q_{L}=0) and work is not required for any treatment unit (W=0). The total enthalpy of a stream is related to the enthalpy of the individual components through Eq. (530) only for streams with known thermodynamic conditions. In addition to the inlet freshwater, each water treatment unit and splitter unit will operate at a known temperature and pressure, so the specific outlet enthalpies of each species, H_{u,u′,s}^{S}, in these units can be determined a priori. Note that Eqs. (529) and (530) suffice to define the enthalpy flow throughout the entire system while leaving degrees of freedom for the heat transfer (Q) to/from the necessary process units.

$$\begin{matrix}{\sum }_{( u , u ′ ) ∈ UC} {H}_{u , u ′}^{T} - {\sum }_{( u ′ , u ) ∈ UC} {H}_{u ′ , u}^{T} - {Q}_{u} - {Q}_{u}^{L} - {W}_{u} = 0 , \\ ∀ u ∈ U / {U}_{Agg} & ( 529 ) \\ {H}_{u , u ′}^{T} - {\sum }_{( u , u ′ , s ) ∈ {S}^{UF}} {H}_{u , u ′ , s}^{S} = 0 , ∀ ( u , u ′ ) ∈ UC & ( 530 )\end{matrix}$$

### Example 6.3.2

### Sour Stripper

In addition to the general mass/energy constraints, specific constraints must be written to govern the operation of each treatment unit, the steam cycle, and the cooling water cycle. The sour stripper is modeled with specified recovery fraction of water in the bottoms, rf_{SS,H}_{₂}_{O}, as in Eq. (531). In this study, the recovery of water is set to 0.9999. For this recovery fraction of water, the composition of the stripper bottoms, is assumed to be known and set using Eq. (532). It is assumed that the recovery of all entrained vapor except NH₃ will be 100%, so the concentration of each of these species is equal to zero. The concentration of NH₃ in the liquid effluent is assumed to be 2×10⁻⁷ mol NH₃/mol.

$$\begin{matrix}{N}_{SS , {SP}_{SS} , {H}_{2} O}^{S} - {rf}_{SS , {H}_{2} O} \cdot {\sum }_{( u , SS ) ∈ UC} {N}_{u , SS , s}^{S} = 0 & ( 531 ) \\ {N}_{SS , {SP}_{SS} , s}^{S} - {x}_{SS , {SP}_{SS} , s}^{Kn} \cdot {N}_{SS , {SP}_{SS} , s}^{T} = 0 , \\ ∀ ( SS , {SP}_{SS} , s ) ∈ {S}^{UF} & ( 532 )\end{matrix}$$

The heat evolved from partial condensation (Q_{SS}^{Cond}) and needed for reboiling (Q_{SS}^{Reb}) are determined from the heat balance across the sour stripper (Eq. (533)). The ratio of the two sour stripper heating values (HR_{SS}) is set using Eq. (534). Based on the analysis of multiple Aspen Plus v7.2 simulations, the value of HR_{SS} was set to 1.21.

Q_{SS}^{Reb}+Q_{SS}^{Cond}−Q_{SS}=0 (533)

HR_{SS}·Q_{SS}^{Reb}+Q_{SS}^{Cond}=0 (534)

### Example 6.3.3

### Biological Digestor

The biological digestor operates at 35° C. and is modeled with a 100% conversion of input feed to biogas (i.e., CH₄, CO₂, H₂, NH₃, and Ar). After implementing the atomic balances around the unit, only one additional constraint is required to determine proper operation of the unit. Eq. (535) will constrain the fraction of the inlet carbon to either CO₂ or CH₄. The amount of carbon typically present as CH₄ in the biogas ranges from 50% to 65%, so it is assumed that 65% of the inlet carbon to the digestor is present as CH₄ (cr_{BD}=0.5385).

N_{BD,CC,CH}_{₄}^{S}−cr_{BD}·N_{BD,CC,CO}_{₂}^{S}=0 (535)

### Example 6.3.4

### Reverse Osmosis

The reverse osmosis unit will remove a given fraction (rf_{RO}) of the total solids (S_{Sol}) in the inlet stream, as shown by Eq. (536).

N_{RO,SP}_{_{RO}}_{,s}^{S}−rf_{RO}·N_{MX}_{_{RO}}_{,RO,s}^{S}=0∀s∈S_{Sol} (536)

### Example 6.3.5

### Cooling Cycle

The circulating flow of cooling water for the CBGTL refinery will be determined from the process cooling requirement (QC), as shown in Eq. (537). The cooling tower will reduce the temperature of the inlet water to 25° C. for use in process cooling. The heat requirement for the cooling water (hr_{COOL-P}) will be calculated as the energy needed to heat the water from 25° C. to 55° C.

Q_{C}−hr_{COOL-P}·N_{CLTR,COOL-P,H}_{₂}_{O}^{S}=0 (537)

To cool the inlet water to the tower, a portion will be evaporated and lost to the atmosphere. To calculate this evaporative loss, the correlation in Eq. (538) is used.

N_{CLTR}^{Evap}−0.00085·ΔT_{CLTR}·N_{CLTR,COOL-P,H}_{₂}_{O}=0 (538)

The temperature difference between the feed to the cooling tower and the outlet water (ΔTCLTR) is assumed to be 30° C. Drift loss from the tower is set to be 0.1% of the inlet flow rate, as in Eq. (539).

N_{CLTR}^{Drift}−0.001·N_{MX}_{_{CLTR}}_{,CLTR,H}_{₂}_{O}^{S}=0 (539)

The total water lost to the atmosphere is equivalent to the drift and evaporation losses, as shown in Eq. (540).

N_{CLTR}^{Evap}+N_{CLTR}^{Drift}−N_{CLTR,OUT}_{_{V}}_{,H}_{₂}_{O}^{S}=0 (540)

During operation of the cooling tower, salts and dissolved solids will begin to accumulate in the circulating water that could foul the heat exchangers and impact heat transfer. A blowdown stream is therefore used to remove these solids from the tower for proper treatment. The flow rate and concentration of this blowdown stream is dependent on the cycles of concentration (COC) used to operate the tower. Cycles of concentration are defined as the ratio of the concentration of solids in the blowdown to the concentration of solids in the inlet. Using typical values of COC in industrial practice, the concentrations of suspended solids and dissolved solids in the cooling tower blowdown are 50 ppm and 2500 ppm, respectively. These quantities are set in Eq. (541) using the known compositions (x_{CLTR,SP}_{_{CLTR}}_{,s}^{Kn})

x_{CLTR,SP}_{_{CLTR}}_{,s}^{Kn}·N_{CLTR,SP}_{_{CLTR}}^{T}−N_{CLTR,SP}_{_{CLTR}}_{,s}^{S}=0,∀s∈S_{Sol} (541)

### Example 6.3.6

### Steam Cycle

The working fluid in the heat engines requires steam generation to produce electricity through the steam turbines. Additionally, steam is required for several process units (i.e., gasifiers, water-gas-shift, and auto-thermal reactor). During steam generation, operation of the boilers will result in accumulation of solids that can impact heat transfer or unit operation. Similar to the cooling tower, a blowdown stream will be utilized that helps contain the amount of dissolved solids in the working heat engine fluid. Using typical values of COC, the solids concentration in the blowdown stream will be 10 ppm for suspended solids and 500 ppm for dissolved solids. This is enforced for process steam generation in Eq. (542) and for heat engine steam generation in Eq. (543).

x_{X}_{_{PWB}}_{,MX}_{_{BLR}}_{,s}^{Kn}·N_{X}_{_{PWB}}_{,MX}_{_{BLR}}^{T}−N_{X}_{_{PWB}}_{,MX}_{_{BLR}}_{,s}^{S}=0,∀s∈S_{Sol} (542)

x_{HEP,MX}_{_{BLR}}_{,s}^{Kn}·N_{HEP,MX}_{_{BLR}}^{T}−N_{HEP,MX}_{_{BLR}}_{,s}^{S}=0,∀s∈S_{Sol} (543)

### Example 6.3.7

### Outlet Wastewater

The contaminants in the wastewater leaving the CBGTL refinery must be less than the maximum concentrations specified for wastewater regulations (x_{MX}_{_{WW}}_{,OUT}_{_{V}}_{,s}^{Max}). This is constrained for total dissolved solids and NH₃ (S_{WW}) using Eq. (544). The maximum allowable concentration of dissolved solids is 500 ppm and for NH₃ is 1 ppm.

### Example 6.3.8

### Unit Costs

The total direct costs, TDC, for the CBGTL refinery wastewater treatment are calculated using the cost parameters in Table 69 and Eq. (545)

$$TDC = ( 1 + BOP ) \cdot {C}_{o} \cdot \frac{{S}^{sf}}{{S}_{o}} \qquad ( 545 )$$

where Cₒ is the base cost, Sₒ is the base capacity, S is the actual capacity, sf is the cost scaling factor, and BOP is the balance of plant (BOP) percentage (site preparation, utility plants, etc.). BOP is calculated as a function of the feedstock higher heating value using Eq. (546).

$$BOP ( \% ) = \frac{0.8867}{M {W}_{HHV}^{0.2096}} \qquad ( 546 )$$

All numbers are converted to 2009 Q4 dollars using the GDP inflation index (US Government Printing Office, 2009, which is incorporated herein by reference as if fully set forth).

The total overnight capital, TOC, for each unit is calculated as the sum of the total direct capital, TDC, plus the indirect costs, IC. The IC include engineering, startup, spares, royalties, and contingencies and is estimated to be 32% of the TDC. The TOC for each unit must be converted to a levelized cost to compare with the variable feedstock and operational costs for the process. Using the methodology of Kreutz et al. (2008), which is incorporated herein by reference as if fully set forth, the capital charges (CC) for the refinery are calculated by multiplying the levelized capital charge rate (LCCR) and the interest during construction factor (IDCF) by the total overnight capital (Eq. (547)).

CC=LCCR·IDCF·TOC (547)

Kreutz et al. (2008), which is incorporated herein by reference as if fully set forth, calculates an LCCR value of 14.38%/yr and IDCF of 1.076. Thus, a multiplier of 15.41%/yr is used to convert the overnight capital into a capital charge rate. Assuming an operating capacity (CAP) of 330 days/yr and operation/maintenance (OM) costs equal to 4% of the TOC, the total levelized cost (CostU) associated with a wastewater unit is given by Eq. 548.

$${Cost}_{u}^{U} = \frac{{CC}_{u} \cdot ( 1 + OM )}{CAP \cdot Prod \cdot {LHV}_{Prod}} \qquad ( 548 )$$

The levelized costs for treatment units used in the wastewater network are then added to the complete list of CBGTL process units. Note that the cost of the cooling utility is now being more rigorously calculated using cooling tower costs. Additionally, the objective function to calculate the total fuels cost will no longer have a Cost^{CW} term.

### Example 6.3.9

### Objective Function

The objective function for the model is given by Eq. (549). The summation represents the total cost of liquid fuels production and includes contributions from the feedstocks cost (Cost^{F}), the electricity cost (Cost^{El}), the CO₂ sequestration cost (Cost^{Seq}), and the levelized unit investment cost (Cost^{U}). Each of the terms in Eq. (549) is normalized to the total lower heating value in GJ of products produced. Note that other objective functions (e.g., maximizing the net present value) can be easily incorporated into the model framework.

$$MIN {\sum }_{u ∈ {U}_{In}} {\sum }_{( u , s ) ∈ {S}^{U}} {Cost}_{s}^{F} + {Cost}^{El} + {Cost}^{Seq} + {\sum }_{u ∈ {U}_{Inv}} {Cost}_{u}^{U} \qquad ( 549 )$$

The process synthesis model with simultaneous heat and power integration, (P), and water integration (Eqs. (531)-(549) represent a large-scale non-convex mixed-integer non-linear optimization (MINLP) model that was solved to generate high-quality locally optimal solutions using either a commercial local MINLP solver or by iteratively fixing the binary variables and solving the resulting non-linear optimization (NLP) model using a commercial NLP solver such as CONOPT. Generation of the local solutions utilizes a large amount (100 250) of starting points for the NLP or MINLP solvers. This procedure determines a variety of local solutions, and the solution with the lowest overall objective value will be used to determine the topological superstructure for the CBGTL facility. The model contains 42 binary variables, 25,700 continuous variables, 336 nonlinear and nonconvex terms, and 25,444 constraints. A theoretical guarantee of the identification of the global optimum may be achieved using a global optimization branch and bound method where valid convex underestimators are introduced for the nonconvex terms.

### Example 7

### Biomass to Liquid Transportation Fuels (BTL) Systems: Process Synthesis and Global Optimization Framework

This example introduces the implementation of the invention, including the process superstructure to convert feedstock to liquid transportation fuels, the simultaneous heat, power, and water integration, and the global optimization algorithm to generate the optimal refinery topologies, applied to biomass to liquid (BTL) systems with agricultural residues, forest residues, and perennial grasses as feedstock. The refineries can range from 1000-200,000 barrels per day capacities and the fuel product ratios can be maximized to produce mainly gasoline, diesel, or jet fuel. The overall greenhouse gas emissions can be less than 50% of petroleum-based processes.

### Example 8

### Biomass and Natural Gas to Liquid Transportation Fuels (BGTL): Process Synthesis, Global Optimization, and Topology Analysis

This example introduces the implementation of the invention, including the process superstructure to convert feedstock to liquid transportation fuels, the simultaneous heat, power, and water integration, and the global optimization algorithm to generate the optimal refinery topologies, applied to biomass and natural gas to liquid (BGTL) systems with agricultural residues, forest residues, and perennial grasses as the biomass feedstock. The refineries can range from 1000-200,000 barrels per day capacities and the fuel product ratios can be maximized to produce mainly gasoline, diesel, or jet fuel.

### Example 9

### Hardwood Biomass to Gasoline, Diesel, and Jet Fuel: I. Process Synthesis and Global Optimization of a Thermochemical Refinery

This example introduces the implementation of the invention, including the process superstructure to convert feedstock to liquid transportation fuels, the simultaneous heat, power, and water integration, and the global optimization algorithm to generate the optimal refinery topologies, applied to hardwood biomass to liquid (BTL) systems. The refineries can range from 800-10,000 barrels per day capacities and the fuel product ratios can be maximized to produce mainly gasoline, diesel, or jet fuel.

### Example 10

### Thermochemical Conversion of Duckweed Biomass to Gasoline, Diesel, and Jet Fuel: Process Synthesis and Global Optimization

This example introduces the implementation of the invention, including the process superstructure to convert feedstock to liquid transportation fuels, the simultaneous heat, power, and water integration, and the global optimization algorithm to generate the optimal refinery topologies, applied to duckweed biomass to liquid (BTL) systems. Aquatic biomass such as duckweed can be produced continually and harvested with simple and low cost mechanical techniques. The refineries can range from 1000-5000 barrels per day capacities and the fuel product ratios can be maximized to produce mainly gasoline, diesel, or jet fuel.

TABLE 69
CBGTL refinery wastewater treatment reference capacities, costs (2009 Q4 $), and scaling factors.

| Description | C_{O} (MM$) | S_{O} | S_{Max} | Units | Scale basis | sf | BOP | Ref. |
|---|---|---|---|---|---|---|---|---|
| Sour stripper | $3.992 | 11.52 | — | kg/s | Feed | 0.53 | — | ^{a} |
| Biological digestor | $4.752 | 115.74 | — | kg/s | Feed | 0.71 | — | ^{b} |
| Reverse osmosis | $0.317 | 4.63 | — | kg/s | Feed | 0.85 | — | ^{b} |
| Cooling tower | $4.055 | 4530.30 | — | kg/s | Feed | 0.78 | — | ^{a} |

^{a} National Energy Technology Laboratory (2007).
^{b} Balmer and Mattson (1994).

TABLE 70
Process unit names, ASPEN block types, and operating assumptions for syngas generation.

| | | | |
|---|---|---|---|
| Unit | ASPEN | | |
| Name | model | Unit Description | Operating Assumptions |

Syngas Generation - Biomass Gasification

| | | | |
|---|---|---|---|
| P101H | Heater | Air Preheater | Tout = 450° F., dP = −0.025 bar |
| P101 | USER2 | Biomass Dryer | Tout, air = 102° C., Tout, biomass = 98° C., Pout = 1 bar, moisture to 15 wt % |
| P102M | Mixer | Biomass | P = 32 bar, CO2/dry biomass = 0.1 wt/wt |
| | | Lockhopper | |
| P102 | USER2 | Biomass Gasifier | Top = Tout = 1000° C., P = 31 bar, inlet O2/dry biomass = 0.3 wt/wt, inlet |
| | | | H2O/dry biomass = 0.25 wt/wt |
| P103S1 | Sep | Primary Gasifier | Outlet to P103M1: 99% of solids, 0% of vapors; dP = 0 bar |
| | | Cyclone | |
| P103S2 | Sep | Secondary Gasifier | Outlet to P103M1: 100% of solids, 0% of vapors; dPsolid = 0 bar, dPvapor = −1 bar |
| | | Cyclone | |
| P103M1 | Mixer | Gasifier Solids | dP = 0 bar |
| | | Mixer | |
| P103 | RStoic | Tar Cracker | dH = 0 kW, dP = −1 bar, reactions given in Table 4 of text |
| P103CL | ClChng | Stream Class | N/A |
| | | Changer | |
| P103M2 | Mixer | Syngas Mixer | dP = 0 bar |

Syngas Generation - Coal Gasification

| | | | |
|---|---|---|---|
| P104H | Heater | Air Preheater | Tout = 450° F., dP = −0.025 bar |
| P104H | USER2 | Coal Dryer | Tout, air = 102° C., Tout, coal = 98° C., Pout = 1 bar, moisture to 2 wt % |
| P105M1 | Mixer | Coal Lockhopper | P = 32 bar, CO2/dry coal = 0.1 wt/wt |
| P105 | USER2 | Coal Gasifier | Top = 1427° C., Tout = 891° C., P = 31 bar, inlet O2/dry coal = 0.7 wt/wt, inlet |
| | | | H2O/dry coal = 0.3 wt/wt |
| P106S1 | Sep | Ash Separator | Outlet to P106M2: 99% of solids, 0% of vapors; dP = 0 bar |
| P106S2 | Sep | Fly-ash Separator | Outlet to P106M2: 100% of solids, 0% of vapors; dPsolid = 0 bar; dPvapor = −1 bar |
| P106M2 | Mixer | Solids Mixer | dP = 0 bar |
| P106CL | ClChng | Stream Class | N/A |
| | | Changer | |

Syngas Generation - Air Separation Unit

| | | | |
|---|---|---|---|
| P501CM1 | MCompr | Air Compressor | 3 stages, Pout = 190 psia, Tcool, 1 = 35° C., Tcool, 2 = 35° C., dPcool = −0.1 bar, |
| | | | η_isen = 0.75, η_mech = 0.95 |
| P501 | Sep | Air/Oxygen | O2 recov. = 100%, O2 outlet: 99.56 wt % O2, 0.43 wt % N2, 0.01 wt % Ar |
| | | Separator | |
| P501SP | FSplit | Oxygen Splitter | dP = 0 bar |
| P501CM2 | MCompr | Oxygen | 2 stages, Pout = 32 bar, Tcomp, 2 = 200° C., dPcool = −0.1 bar, η_isen = 0.75, |
| | | Compressor | η_mech = 0.95 |

TABLE 71
Process unit names, ASPEN block types, and operating assumptions for syngas cleaning.

| | | | |
|---|---|---|---|
| Unit | ASPEN | | |
| Name | model | Unit Description | Operating Assumptions |

Syngas Cleaning - Acid Gas Removal and CO₂ Recycle

| | | | |
|---|---|---|---|
| P201 | RGibbs | Reverse Water Gas | T = 700° C., dP = −0.5 bar |
| | | Shift Reactor | |
| P201SP | FSplit | H₂ Splitter | dP = 0 bar |
| P201H1 | Heater | Hydrogen Preheater | T = 700° C., dP = −0.5 bar |
| P201H2 | Heater | Oxygen Preheater | T = 700° C., dP = −0.5 bar |
| P202H | Heater | Primary Gas Cooler | Tₒᵤₜ = 185° C., dP = −0.5 bar |
| P202 | RGibbs | COS/HCN Hydrolyzer | dH = 0 kW, dP = −0.5 bar |
| P203 | Sep | NH3/HCl Scrubber | 100% NH3, HCl separation |
| P203H | Heater | Secondary Gas Cooler | Tₒᵤₜ = 35° C., dP = −0.5 bar |
| P204F | Flash2 | Water Knock Out Unit | dH = 0 kW, dP = −0.5 bar |
| P204M1 | Mixer | Acid Gas Mixer | dP = 0 bar |
| P204M1 | Heater | Thermal Analyzer | Tₒᵤₜ = 12° C., dP = 0 bar |
| P204 | Sep | Rectisol Unit Primary | Conditions given in Table 5 of text |
| | | Stage | |
| P204CM1 | Compr | CO₂ Initial | Pₒᵤₜ = 3 bar, ηᵢₛₑₙ = 0.75, η_{mech} = 0.95 |
| | | Compressor | |
| P204M2 | Mixer | CO₂ Mixer | dP = 0 bar |
| P204CM2 | MCompr | CO₂ Recycle | 3 stages, Pₒᵤₜ = 32 bar, T_{comp,2} = 200° C., T_{comp,3} = 200° C., dP_{cool} = −0.1 bar, |
| | | Compressor | ηᵢₛₑₙ = 0.75, η_{mech} = 0.95 |
| P204SP | FSplit | CO₂ Splitter | dP = 0 bar |
| P204H2 | Heater | CO₂ Preheater | Tₒᵤₜ = 700° C., dP = −0.5 bar |
| P204CM3 | Compr | Acid Gas Compressor | Pₒᵤₜ = 2 bar, ηᵢₛₑₙ = 0.75, η_{mech} = 0.95 |

Syngas Cleaning - Claus Treatment Plant

| | | | |
|---|---|---|---|
| P206H1 | Heater | Acid Gas Preheater | Tₒᵤₜ = 450° F., dP = −0.025 bar |
| P206SP | FSplit | Claus Furnace | Split fraction adjusted so H₂S/CO₂ = 2 for P207 inlet |
| | | Splitter | |
| P206H2 | Heater | Oxygen Preheater | Tₒᵤₜ = 450° F., dP = −0.025 bar |
| P206143 | Heater | Sour Gas Preheater | Tₒᵤₜ = 450° F., dP = −0.025 bar |
| P206 | RStoic | Claus Furnace | T = 1350° C., dP = −0.025 bar, O_{2,inlet}/O_{2,stoic} = 1.2 |
| P207 | RStoic | First Claus Converter | T = 650° F., dP = −0.025 bar, FC__{H2S} = 0.625 |
| P208 | Sep | First Sulfur Separator | Tₒᵤₜ = 380° F., dP = −0.025 bar, all sulfur to P207M, all vapors to P209H |
| P209H | Heater | Second Claus | Tₒᵤₜ = 450° F., dP = −0.025 bar |
| | | Preheater | |
| P209 | RStoic | Second Claus | dH = 0 kW, dP = −0.025 bar, FC_{—H2S} = 0.9 |
| | | Converter | |
| P210 | Sep | Second Sulfur | Tₒᵤₜ = 350° F., dP = −0.025 bar, all sulfur to P207M, all vapors to P211H |
| | | Separator | |
| P211H | Heater | Third Claus Preheater | Tₒᵤₜ = 420° F., dP = −0.025 bar |
| P211 | RStoic | Third Claus Converter | dH = 0 kW, dP = −0.025 bar, FC_{—H2S} = 0.9 |
| P212 | Sep | Third Sulfur | Tₒᵤₜ = 320° F., dP = −0.025 bar, all sulfur to P207M, all vapors to |
| | | Separator | P213H1 |
| P213H1 | Heater | Hydrolyzer Preheater | Tₒᵤₜ = 450° F., dP = −0.025 bar |
| P213 | RGibbs | Claus Hydrolyzer | dH = 0 kW, dP = −0.025 bar |
| P207M | Mixer | Sulfur Pit | dP = 0 bar |
| P213H2 | Heater | Tail Gas Cooler | Tₒᵤₜ = 35° C., dP = −0.025 bar |
| P213F | Flash2 | Claus Water Knock | dH = 0 kW, dP = −0.025 bar |
| | | Out | |
| P213M | Mixer | Claus Water Mixer | dP = 0 bar |
| P213CM | MCompr | Tail Gas Compressor | 3 stages, Pₒᵤₜ = 25 bar, T_{cool,1} = 35° C., T_{cool,2} = 35° C. dP_{cool} = −0.1 bar, |
| | | | ηᵢₛₑₙ = 0.75, η_{mech} = 0.95 |

Syngas Cleaning - Water Recovery

| | | | |
|---|---|---|---|
| P205M | Mixer | Sour Stripper Mixer | dP = 0 bar |
| P205F1 | Flash2 | First Sour Water | dH = 0 kW, dP = −0.025 bar |
| | | Knockout | |
| P205F2 | Flash2 | Second Sour Water | dH = 0 kW, dP = −0.025 bar |
| | | Knockout | |
| P205 | RadFrac | Sour Stripper Column | Nₜₒₜₐₗ = 10, N_{feed} = 1, Nᵥₐₚ = 1, liq. N_{liq} = 10, no cond., P₁ = 1.01 bar, dP_{col} = |
| | | | −0.3 bar |
| P205CM | Compr | Sour Gas Compressor | Pₒᵤₜ = 30 psia, ηᵢₛₑₙ = 0.75, η_{mech} = 0.95 |

TABLE 72
Process unit names, ASPEN block types, and operating assumptions for liquid fuel generation.

| | | | |
|---|---|---|---|
| Unit | ASPEN | | |
| Name | model | Unit Description | Operating Assumptions |

Liquid Fuel Generation - Hydrocarbon Production

| | | | |
|---|---|---|---|
| P301H | Heater | Vaporizer | Vapor fraction = 1, dP = −0.5 bar |
| P301CM | Compr | FT Compressor | Pout = 24.4 bar, η_isen = 0.75, η_mech = 0.95 |
| P301SP | FSplit | FT Splitter | dP = 0 bar |
| P301BH | Heater | High Temp. FT Preheater | Tout = 320° C., dP = −0.5 bar |
| P301B | USER2 | High Temp. FT Reactor | T = 320° C., dP = −1.5 bar, FC_CO = 0.8, α = 0.65 |
| P301AH | Heater | Low Temp. FT Preheater | Tout = 240° C., dP = −0.5 bar |
| P301A | USER2 | Low Temp. FT Reactor | T = 240° C., dP = −1.5 bar, FC_CO = 0.8, α = 0.73 |
| P301M | Mixer | FT Effluent Mixer | dP = 0 bar |
| P302 | Flash2 | FT Wax Separator | dH = 0 kW, dP = −0.5 bar |
| P302H | Heater | Wax Cooler | Tout = 150° C., dP = −0.5 bar |
| P303H | Heater | Hydrocarbon Cooler | Tout = 40° C., dP = −0.5 bar |
| P303 | Sep | Aqueous Oxygenate | dP = −0.5 bar, 100% separation of aqueous oxygenates to |
| | | Separator | P303M |
| P303M | Mixer | Water Knockout Mixer | dP = 0 bar |
| P304 | Flash3 | Hydrocarbon Water | dH = 0 kW, dP = −0.5 bar |
| | | Knockout | |
| P304M | Mixer | First Hydrocarbon Mixer | dP = 0 bar |
| P305 | Flash2 | Wax Vapor Removal | dH = 0 kW, Pout = 6 bar |
| P305M | Mixer | Second Hydrocarbon Mixer | dP = 0 bar |
| P306H | Heater | Wax Vapor Cooler | Tout = 40° C., dP = −0.25 bar |
| P306 | Flash3 | Vapor Water Knockout | dH = 0 kW, dP = −0.25 bar |
| P307 | Sep | Vapor Oxygenate Separator | dP = −0.5 bar, 100% separation of vapor oxygenates to P303M |

Liquid Fuel Generation - Hydrocarbon Upgrading and Light Gas Reforming

| | | | |
|---|---|---|---|
| P401 | Sep | Hydrocarbon Recovery Unit | Conditions given in Table SI.5 |
| P401SP | FSplit | Kerosene Splitter | dP = 0 bar |
| P401M | Mixer | Sour Water Mixer | dP = 0 bar |
| P402 | USER2 | Wax Hydrocracker | Conditions given in Table SI.5 |
| P403 | USER2 | Distillate Hydrotreater | Conditions given in Table SI.5 |
| P403M | Mixer | Kerosene Cut Mixer | dP = 0 bar |
| P404 | USER2 | Kerosene Hydrotreater | Conditions given in Table SI.5 |
| P405 | USER2 | Naphtha Hydrotreater | Conditions given in Table SI.5 |
| P402M | Mixer | Diesel Blender | dP = 0 bar |
| P406 | USER2 | Naphtha Reformer | Conditions given in Table SI.5 |
| P407M | Mixer | C5/C6 Gases Mixer | dP = 0 bar |
| P407 | USER2 | C5/C6 Isomerizer | Conditions given in Table SI.5 |
| P408 | USER2 | Gasoline Blender | Conditions given in Table SI.5 |
| P411M1 | Mixer | First Light Gas Mixer | dP = 0 bar |
| P411M2 | Mixer | Second Light Gas Mixer | dP = 0 bar |
| P411M3 | Mixer | Third Light Gas Mixer | dP = 0 bar |
| P409 | USER2 | C4 Isomerizer | Conditions given in Table SI.5 |
| P410 | USER2 | C3/C4/C5 Alkylation Unit | Conditions given in Table SI.5 |
| P411 | USER2 | Saturated Gas Plant | Conditions given in Table SI.5 |
| P411SP | FSplit | ATR/Combustion Splitter | dP = 0 bar |

TABLE 73
Process unit names, ASPEN block types, and operating assumptions for light gas reforming.

| Unit | ASPEN | | |
|---|---|---|---|
| Name | model | Unit Description | Operating Assumptions |
| P412CM | Compr | ATR Compressor | Pout = 32 bar, η_isen = 0.75, η_mech = 0.95 |
| P412H1 | Heater | ATR Preheater | Tout = 800° C., dP = −0.5 bar |
| P412 | RGibbs | ATR Unit | T = 950° C., dP = −1.5 bar, H2Oinlet/Cinlet = 0.5 |
| P412H2 | Heater | Steam Preheater | T = 800° C., dP = −0.5 bar |
| P412H3 | Heater | Oxygen Preheater | T = 800° C., dP = −0.5 bar |
| P412H4 | Heater | Natural Gas | T = 800° C., dP = −0.5 bar |
| | | Preheater | |
| P413CM | Compr | Combustion | Pout = 29 bar, η_isen = 0.75, η_mech = 0.95 |
| | | Compressor | |
| P413 | RStoic | Combustor Unit | T = 1300° C., dP = −1.0 bar, Inlet O2/Stoichiometric O2 = 1.2 |
| P413H | Heater | Combustion | Tout = 35° C., dP = −0.5 bar |
| | | Effluent Cooler | |
| P413F | Flash2 | Combustion Water | dH = 0 kW, dP = −0.5 bar |
| | | Knockout | |
| P414H | Heater | Thermal Analyzer | Tout = 12° C., dP = 0 bar |
| P414 | Sep | Rectisol Unit | Conditions given in Table 5 of text |
| P415CM1 | Compr | Light Gas | Pout = 467.5 psia, η_isen = 0.75, η_mech = 0.95 |
| | | Compressor | |
| P415H1 | Heater | Light Gas Heater | T = 385° F., dP = −0.5 bar |
| P415CM2 | Compr | Air Compressor | Polytropic compressor using the ASME method, P_ratio = 19.5, η_polytropic = |
| | | | 0.87, η_mech = 0.9865 |
| P415SP | FSplit | Air Splitter | 0.1% leak, 5% retained air to P415M2, all remaining air to P415 |
| P415M1 | Mixer | Light Gas Mixer | dP = 0 bar |
| P415CM3 | Compr | Air Compressor | Polytropic compressor using the ASME method, P_out = 460 psia, |
| | | | η_polytropic = 0.87, η_mech = 0.9865 |
| P415 | RStoic | Gas Turbine | T = 1370° C., dP = −10 psia, Inlet O2/Stoichiometric O2 = 1.1 |
| | | Combuster | |
| P415T1 | Compr | Gas Turbine 1 | Pout = 235.2 psia, η_isen = 0.89769, η_mech = 0.9727 |
| P415M2 | Mixer | Air Injection Mixer | dP = 0 bar |
| P415T2 | Compr | Gas Turbine 2 | Pout = 1.065 bar, η_isen = 0.89769, η_mech = 0.9727 |
| P415H2 | Heater | Effluent Cooler | T = 35° C., dP = −0.025 bar |
| P415F | Flash2 | Water Knockout | dH = 0 kW, dP = −0.025 bar |
| P415CM4 | Compr | Gas Compressor | Pout = 27.3 bar, η_isen = 0.75, η_mech = 0.95 |
| P415H3 | Heater | Gas Heater | T = 35° C., dP = −0.5 bar |

TABLE 74
Outlet conditions for the upgrading units.

| Unit | | | |
|---|---|---|---|
| Name | Unit Description | Outlet Conditions¹ | Hᵢₙₚᵤₜ/Cᵢₙₚᵤₜ² |
| P401 | Hydrocarbon | T_{LG} = T_{C3-5G} = T_{N} = T_{K} = T_{Dt} = T_{Wx} = T_{WW} = 100° F. | N/A |
| | Recovery Unit | P_{LG} = P_{C3-5G} = P_{N} = P_{K} = P_{Dt} = P_{Wx} = P_{WW} = 50 | |
| | | psia | |
| P402 | Wax Hydrocracker | T_{C5-6G} = 150° F., T_{N} = 200° F., T_{D} = 300° F., T_{SW} = | 2.19516 |
| | | T_{LG} = 100° F. | |
| | | P_{C5-6G} = 60 psia, P_{N} = 40 psia, P_{D} = P_{SW} = 20 | |
| | | psia, P_{LG} = 50 psia | |
| P403 | Distillate | T_{SW} = 80° F., T_{D} = 90° F., T_{LG} = 100° F. | 2.13922 |
| | Hydrotreater | P_{SW} = P_{D} = P_{LG} = 50 psia | |
| P404 | Kerosene | T_{K} = 90° F., T_{LG} = 100° F. | 2.13922 |
| | Hydrotreater | P_{K} = P_{LG} = 50 psia | |
| P405 | Naphtha | T_{SW} = 70° F., T_{C5-6G} = 90° F., T_{N} = 80° F., T_{LG} = | 2.30795 |
| | Hydrotreater | 100° F. | |
| P406 | Naphtha Reformer | P_{SW} = P_{C5-6G} = P_{N} = P_{LG} = 50 psia | N/A |
| | | T_{G} = 150° F., T_{H2G} = 120° F., T_{LG} = 100° F. | |
| | | P_{G} = 30 psia, P_{H2G} = P_{LG} = 50 psia | |
| P407 | C₅/C₆ Isomerizer | T_{G} = T_{LG} = 100° F. | 2.3589 |
| | | P_{G} = P_{LG} = 25 psia | |
| P408 | Gasoline Blender | T_{G} = 100° F. | N/A |
| | | P_{G} = 50 psia | |
| P409 | C₄ Isomerizer | T_{C4G} = T_{LG} = 100° F. | 2.50646 |
| | | P_{C4G} = P_{LG} = 50 psia | |
| P410 | C₃/C₄/C5 Alkylation | T_{G} = T_{C4G} = T_{LG} = 100° F. | N/A |
| | Unit | P_{G} = P_{C4G} = P_{LG} = 50 psia | |
| P411 | Saturated Gas | T_{SW} = T_{C3G} = T_{C4G} = T_{LG} = 100° F. | N/A |
| | Plant | P_{SW} = P_{C3G} = P_{C4G} = P_{LG} = 50 psia | |

¹LG = Light Gas, C3G = C₃ Gases, C3-5G = C₃-C₅ Gases, C4G = C₄ Gases, C5-6G = C₅-C₆ Gases, H2G = H₂ Rich Gases, N = Naphtha, Dt = Distillate, Wx = Wax, WW = Wastewater, SW = Sour Water, G = Gasoline, D = Diesel, K = Kerosene
²Refers to the total present in all input hydrocarbons plus input H₂

### REFERENCES

[1] R. C. Baliban, J. A. Elia, C. A. Floudas. Toward Novel Hybrid Biomass, Coal, and Natural Gas Processes for Satisfying Current Transportation Fuel Demands, 1: Process Alternatives, Gasification Modeling, Process Simulation, and Economic Analysis. Ind. Eng. Chem. Res., 2010:49(16), 7343-7370.
[2] J. A. Elia, R. C. Baliban, C. A. Floudas. Toward Novel Hybrid Biomass, Coal, and Natural Gas Processes for Satisfying Current Transportation Fuel Demands, 2: Simultaneous Heat and Power Integration. Ind. Eng. Chem. Res., 2010:49(16), 7371-7388.
[3] R. C. Baliban, J. A. Elia, C. A. Floudas. Optimization Framework for the Simultaneous Process Synthesis, Heat and Power Integration of a Thermochemical Hybrid Biomass, Coal, and Natural Gas Facility. Comp. Chem. Eng., 2011:35(9), 1647-1690.
[4] J. A. Elia, R. C. Baliban, X. Xiao, C. A. Floudas. Optimal Energy Supply Network Determination and Life Cycle Analysis for Hybrid Coal, Biomass, and Natural Gas to Liquid (CBGTL) Plants Using Carbon-based Hydrogen Production. Comp. Chem. Eng., 2011:35(8), 1399-1430.
[5] R. C. Baliban, J. A. Elia, C. A. Floudas. Simultaneous Process Synthesis, Heat, Power, and Water Integration of Thermochemical Hybrid Biomass, Coal, and Natural Gas Facilities. Comp. Chem. Eng., 2012:37(10), 297.
[6] R. C. Baliban, J. A. Elia, R. Misener, C. A. Floudas. Global Optimization of a Thermochemical Based Hybrid Coal, Biomass, and Natural Gas to Liquids Facility. Comp. Chem. Eng., submitted.
[7] R. C. Baliban, J. A. Elia, C. A. Floudas. Toward Novel Hybrid Biomass, Coal, and Natural Gas Processes for Satisfying Current Transportation Fuel Demands, 1: Process Alternatives, Gasification Modeling, Process Simulation, and Economic Analysis. Ind. Eng. Chem. Res., 2010:49(16), 7343-7370.
[8] J. A. Elia, R. C. Baliban, C. A. Floudas. Toward Novel Hybrid Biomass, Coal, and Natural Gas Processes for Satisfying Current Transportation Fuel Demands, 2: Simultaneous Heat and Power Integration. Ind. Eng. Chem. Res., 2010:49(16), 7371-7388.
[9] R. C. Baliban, J. A. Elia, C. A. Floudas. Optimization Framework for the Simultaneous Process Synthesis, Heat and Power Integration of a Thermochemical Hybrid Biomass, Coal, and Natural Gas Facility. Comp. Chem. Eng., 2011:35(9), 1647-1690.
[10] J. A. Elia, R. C. Baliban, X. Xiao, C. A. Floudas. Optimal Energy Supply Network Determination and Life Cycle Analysis for Hybrid Coal, Biomass, and Natural Gas to Liquid (CBGTL) Plants Using Carbon-based Hydrogen Production. Comp. Chem. Eng., 2011:35(8), 1399-1430.
[11] R. C. Baliban, J. A. Elia, C. A. Floudas. Simultaneous Process Synthesis, Heat, Power, and Water Integration of Thermochemical Hybrid Biomass, Coal, and Natural Gas Facilities. Comp. Chem. Eng., 2012:37(10), 297.
[12] C.A. Floudas, J. A. Elia, R. C. Baliban. Hybrid and Single Feedstock Energy Processes for Liquid Transportation Fuels: A Critical Review. Comp. Chem. Eng., 2012:41, 24-51.
[13] R. C. Baliban, J. A. Elia, R. Misener, C. A. Floudas. Global Optimization of a Thermochemical Based Hybrid Coal, Biomass, and Natural Gas to Liquids Facility. Comp. Chem. Eng., 2012:42, 64.
[14] J. A. Elia, R. C. Baliban, C. A. Floudas. Nationwide Supply Chain Analysis for Hybrid Energy Processes with Significant CO₂ Emissions Reduction. AIChE J., 2012:58(7), 2142-2154.
[15] R. C. Baliban, J. A. Elia, V. W. Weekman, C. A. Floudas. Process synthesis of hybrid coal, biomass, and natural gas to liquids via Fischer-Tropsch synthesis, ZSM-5 catalytic conversion, methanol synthesis, methanol-to-gasoline, and methanol-to-olefins/distillate technologies. Comp. Chem. Eng., in press, doi:10.1016/j.compchemeng.2012.06.032.
[16] Energy Information Administration. Monthly Energy Review, August 2009. Document No. DOE-EIA-0035(2009/09). Available via the Internet at http://www.eta.doe.gov/mer/, 2009.
[17] Energy Information Administration. Annual Energy Outlook 2009 with Projections to 2030. Document No. DOE/EIA-0383 (2009). Available via the Internet at http://www.eta.doe.gov/oiaf/aeo/, 2009.
[18] Zittel, W.; Schindler, J. Crude Oil: The Supply Outlook. Document No. EWG-Series No 3/2007, Report to the Energy Watch Group, October 2007.
[19] BP, BP Statistical ReView of World Energy. Available via the Internet at http://www.bp.com/, 2009.
[20] Environmental Protection Agency, Inventory of U.S. Greenhouse Gas Emission and Sinks: 1990-2007. Document No. EPA 430-R-09-004, Washington, D.C., 2009.
[21] MacLean, H.; Lave, L. Evaluating automobile fuel/propulsion system technologies. Prog. Energy Combust. Sci. 2003, 29, 1-69.
[22] National Research Council and National Academy of Engineering. The Hydrogen Economy: Opportunities, Costs, Barriers, and R&D Needs; The National Academies Press: Washington, D.C., 2004.
[23] Lynd, L. R.; Larson, E.; Greene, N.; Laser, M.; Sheehan, J.; Dale, B. E.; McLaughlin, S.; Wang, M. The role of biomass in America's energy future: framing the analysis. Biofuels, Bioprod. Biorefin. 2009, 3, 113-123.
[24] National Academy of Sciences, National Academy of Engineering, and National Research Council. Liquid Transportation Fuels from Coal and Biomass: Technological Status, Costs, and EnVironmental Issues. Prepublication; EPA: Washington, D.C., 2009.
[25] Department of Energy and Department of Agriculture. Biomass as Feedstock for a Bioenergy and Bioproducts Industry: The Technical Feasibility of a Billion-Ton Annual Supply. Document No. DOE/GO-102005-2135, http://wwwLeere.energy.gov/biomass/publications.html, 2005.
[26] Kreutz, T. G.; Larson, E. D.; Liu, G.; Williams, R. H. Fischer-Tropsch Fuels from Coal and Biomass. Presented at the 25th International Pittsburgh Coal Conference, 2008.
[27] Larson, E. D.; Jin, H. Biomass Conversion to Fischer-Tropsch Liquids: Preliminary Energy Balances. In Proceedings of the 4th Biomass Conference of the Americas, 1999.
[28] Vliet, O.; Faaij, A.; Turkenburg, W. Fischer-Tropsch diesel production in a well-to-wheel perspective: A carbon, energy flow and cost analysis. Energy ConV ers. Manage. 2009, 50, 855-876.
[29] Bechtel Corporation; Global Energy Inc.; Nexant Inc. Gasification Plant Cost and Performance Optimization, Task 2 Topical Report: Coke/Coal Gasification with Liquids Coproduction, USDOE Contract No. DEAC26-99FT40342, 2003.
[30] Sudiro, M.; Bertucco, A. Production of synthetic gasoline and diesel fuel by alternative processes using natural gas and coal: Process simulation and optimization. Energy 2009, 34, 2206-2214.
[31] Cao, Y.; Gao, Z.; Jin, J.; Zhou, H.; Cohron, M.; Zhao, H.; Liu, H.; Pan, W. Synthesis Gas Production with an Adjustable H2/CO Ratio through the Coal Gasification Process: Effects of Coal Ranks and Methane Addition. Energy Fuels 2008, 22, 1720-1730.
[32] Sudiro, M.; Bertucco, A. Synthetic Fuels by a Limited CO₂ Emission Process Which Uses Both Fossil and Solar Energy. Energy Fuels 2007, 21, 3668-3675.
[33] Yamashita, K.; Barreto, L. Energyplexes for the 21st century: Coal gasification for co-producing hydrogen, electricity and liquid fuels. Energy 2005, 30, 2453-2473.
[34] Chiesa, P.; Consonni, S.; Kreutz, T.; Williams, R. Co-production of hydrogen, electricity and CO2 from coal with commercially ready technology. Part A: Performance and emissions. Int. J. Hydrogen Energy 2005, 30, 747-767.
[35] Kreutz, T.; Williams, R.; Consonni, S.; Chiesa, P. Co-production of hydrogen, electricity and CO2 from coal with commercially ready technology. Part B: Economic analysis. Int. J. Hydrogen Energy 2005, 30, 769-784.
[36] Sudiro, M.; Bertucco, A.; Ruggeri, F.; Fontana, M. Improving Process Performances in Coal Gasification for Power and Synfuel Production. Energy Fuels 2008, 22, 3894-3901.
[37] Larson, E. D.; Jin, H.; Celik, F. E. Large-scale gasification-based coproduction of fuels and electricity from switchgrass. Biofuels, Bioprod. Bioref. 2009, 3, 174-194.
[38] Cormos, C. C. Assessment of hydrogen and electricity co-production schemes based on gasification process with carbon capture and storage. Int. J. Hydrogen Energy 2009, 34, 6065-6077.
[39] Jiménez, L.; Gadalla, M.; Boer, D.; Majozi, T. Integrated gasification combined cycle (IGCC) process simulation and optimization. Comput. Chem. Eng. 2009, DOI: 10.1016/j.compchemeng.2009.04.007.
[40] Agrawal, R.; Singh, N. R.; Ribeiro, F. H.; Delgass, W. N. Sustainable fuel for the transportation sector. Proc. Natl. Acad. Sci. U.S.A. 2007, 104, 4828-4833.
[41] Dry, M. The Fischer-Tropsch process:1950-2000. Catal. Today 2002, 71, 227-241.
[42] National Energy Technology Laboratory. Cost and Performance Baseline for Fossil Energy Plants. Volume 1: Bituminous Coal and Natural Gas to Electricity Final Report. Document No. DOE/NETL-2007/1281, http://www.netl.doe.gov/energy-analyses/baselinetudies.html, 2007.
[43] Bechtel, Aspen Process Flowsheet Simulation Model of a Battelle Biomass-Based Gasification, Fischer-Tropsch Liquefaction and Combined-Cycle Power Plant, Contract No. DE-AC22-93PC91029, http://www.Fischer-Tropsch.org/, 1998.
[44] Bechtel, Baseline Design/Economics for Advanced Fischer-Tropsch Technology, Contract No. DE-AC22-91PC90027, 1992.
[45] Hamelinck, C.; Faaij, A.; Uil, H.; Boerrigter, H. Production of FT transportation fuels from biomass; technical options, process analysis and optimization, and development potential. Energy 2004, 29, 1743.
[46] Spath, P. Aden, A. Eggeman, T. Ringer, M. Wallace, B. Jechura, J. Biomass to Hydrogen Production Detailed Design and Economics Utilizing the Battelle Columbus Laboratory Indirectly-Heated Gasifier. Document No. NREL/TP-510-37408, http://www.nrel.gov/docs/fy05osti/37408.pdf, 2005.
[47] Holladay, J. D.; Hu, J.; King, D. L.; Wang, Y. An overview of hydrogen production technologies. Catal. Today 2009, 139, 244-260.
[48] Floudas, C. A. Nonlinear and Mixed-Integer Optimization; Oxford University Press, New York: 1995.
[49] Holiastos, K.; Manousiouthakis, V. Minimum hot/cold/electric utility cost for heat exchange networks. Comput. Chem. Eng. 2002, 26, 3-16.
[50] Li, X. T.; Grace, J. R.; Watkinson, A. P.; Ergüdenler, A. Equilibrium modeling of gasification: A free energy minimization approach and its application to a circulating fluidized bed coal gasifier. Fuel 2001, 80, 195-207.
[51] Yuehong, Z.; Hao, W.; Zhihong, X. Conceptual design and simulation study of a co-gasification technology. Energy ConVers. Manage. 2006, 47, 1416-1428.
[52] Ranzi, E.; Cuoci, A.; Faravelli, T.; Frassoldati, A.; Migliavacca, G.; Pierucci, S.; Sommariva, S. Chemical Kinetics of Biomass Pyrolysis. Energy Fuels 2008, 22, 4292-4300.
[53] Cuoci, A.; Faravelli, T.; Frassoldati, A.; Granata, S.; Migliavacca, G.; Ranzi, E.; Sommariva, S. A General Mathematical Model of Biomass Devolatilization Note 1. Lumped kinetic models of cellulose, hemicellulose, and lignin, Presented at the 30th Meeting of the Italian Section of the Combustion Institute, 2007.
[54] Jand, N.; Brandani, V.; Foscolo, P. U. Thermodynamic Limits and Actual Product Yields and Compositions in Biomass Gasification Processes. Ind. Eng. Chem. Res. 2006, 45, 834-843.
[55] Ratnadhariya, J. K.; Channiwala, S. A. Three zone equilibrium and kinetic free modeling of biomass gasifiers A novel approach. Renewable Energy 2009, 34, 1050-1058.
[56] Nikoo, M. B.; Mahinpey, N. Simulation of biomass gasification in a fluidized bed reactor using ASPEN PLUS. Biomass Bioenergy 2008, 32, 1245-1254.
[57] Yang, H.; Yan, R.; Chen, H.; Lee, D. H.; Zheng, C. Characteristics of hemicellulose, cellulose, and lignin pyrolysis. Fuel 2007, 86, 1781-1788.
[58] Narvaez, I.; Orio, A.; Aznar, M. P.; Corella, J. Biomass Gasification with Air in an Atmospheric Bubbling Fluidized Bed. Effect of Six Operational Variables on the Quality of the Produced Raw Gas. Ind. Eng. Chem. Res. 1996, 35, 2110-2120.
[59] Gil, J.; Aznar, M. P.; Caballero, M. A.; Frances, E.; Corella, J. Biomass Gasification in Fluidized Bed at Pilot Scale with Steam-Oxygen Mixtures. Product Distribution for Very Different Operating Conditions. Energy Fuels 1997, 11, 1109-1118.
[60] Gil, J.; Corella, J.; Aznar, M. P.; Caballero, M. A. Biomass gasification in atmospheric and bubbling fluidized bed: Effect of the type of gasifying agent on the product distribution. Biomass Bioenergy 1999, 17, 389-403.
[61] Yoon, H.; Wei, J.; Denn, M. M. A Model For Moving-Bed Coal Gasification Reactors. AIChE J. 1978, 24, 885-903.
[62] Song, B. H.; Jang, Y. W.; Kim, S. D.; Kang, S. K. Gas Yields from Coal Devolatilization in a Bench-Scale Fluidized Bed Reactor. Korean J. Chem. Eng. 2001, 18, 770-774.
[63] Ma, R. P.; Felder, R. M.; Ferrell, J. K. Evolution of Hydrogen Sulfide in a Fluidized Bed Coal Gasification Reactor. Ind. Eng. Chem. Res. 1989, 28, 27-33.
[64] Di Nola, G.; de Jong, W.; Spliethoff, H. The fate of main gaseous and nitrogen species during fast heating rate devolatilization of coal and secondary fuels using a heated wire mesh reactor. Fuel Process. Technol. 2009, 90, 388-395.
[65] Liu, H. F.; Liu, Y. H.; Liu, Y. H.; Che, D. F. Experimental investigation on the conversion of nitrogenous gas products during coal pyrolysis. J. Fuel Chem. Technol. 2008, 36, 134-138.
[66] Mannan, S. Lees' Loss PreVention in the Process Industries, Vols. 1-3; Elsevier: 2005. (Online version available at http://knovel.com/).
[67] Fiaschi, D.; Michelini, M. A two-phase one-dimensional biomass gasification kinetics model. Biomass Bioenergy 2001, 21, 121-132.
[68] Sotudeh-Gharebaagh, R.; Legros, R.; Paris, J. Simulation of circulating fluidized bed reactors using ASPEN PLUS. Fuel 1998, 77, 327-337.
[69] NASA, http://cea.grc.nasa.gov/.
[70] Amundson, N. R.; Arri, L. E. Char Gasification in a Countercurrent Reactor. AIChE J. 1978, 24, 87-101.
[71] Herguido, J.; Corella, J.; Gonzalez-Saiz, J. Steam Gasification of Lignocellulosic Residues in a Fluidized Bed at a Small Pilot Scale. Effect of the Type of Feedstock. Ind. Eng. Chem. Res. 1996, 31, 1274-1282.
[72] Yu, Q.-Z.; Brage, C.; Chen, G.-X.; Sjostrom, K. The fate of fueInitrogen during gasification of biomass in a pressurized fluidized bed gasifier. Fuel 2007, 86, 611-618.
[73] Zhou, J. Simulation of fuel-bound nitrogen evolution in biomass gasification. In Proceedings of the 32nd Intersociety Energy Conversion Engineering Conference, 1997.
[74] Liu, H.; Gibbs, B. M. Modeling NH3 and HCN emissions from biomass circulating fluidized bed gasifiers. Fuel 2003, 82, 1591-1604.
[75] Stubenberger, G.; Scharler, R.; Zahirovic, S.; Obernberger, I. Experimental investigation of nitrogen species release from different solid biomass fuels as a basis for release models. Fuel 2008, 87, 793-806.
[76] van der Drift, A.; van Doorn, J.; Vermeulen, J. W. Ten residual fuels for circulating fluidized-bed gasification. Biomass Bioenergy 2001, 20, 45-56.
[77] Zwart, R. W. R.; Boerrigter, H. High Efficiency Co-production of Synthetic Natural Gas (SNG) and Fischer-Tropsch (FT) Transportation Fuels from Biomass. Energy Fuels 2005, 19, 591-597.
[78] Oukaci, R. Fischer-Tropsch Synthesis. In 2nd Annual Global GTL Summit ExecutiVe Briefing, May 28-30, 2002, London, U.K.
[79] Bechtel, Baseline Design/Economics for Advanced Fischer-Tropsch Technology. Quarterly Report, January-March 1993, Contract No. DE-AC22-91PC90027, 1993.
[80] Chem. Eng. Mag. Monthly, http://www.che.com/pci/, 2009.
[81] Hsi, C. L.; Wang, T. Z.; Tsai, C. H.; Chang, C. Y.; Liu, C. H.; Chang, Y. C.; Kuo, J. T. Characteristics of an Air-Blown Fixed-Bed Downdraft Biomass Gasifier. Energy Fuels 2008, 22, 4196-4205.
[82] Faaij, A.; Ree, R. V.; Waldheim, L.; Olsson, E.; Oudhuis, A.; Wijk, A. V.; Daey-Ouwens, C.; Turkenburg, W. Gasification of biomass wastes and residues for electricity production. Biomass Bioenergy 1997, 12, 387-407.
[83] Jayah, T. H.; Aye, L.; Fuller, R. J.; Stewart, D. F. Computer simulation of a downdraft wood gasifier for tea drying. Biomass Bioenergy 2003, 25, 459-469.
[84] Watkinson, A. P.; Lucas, J. P.; Lim, C. J. A prediction of performance of commercial coal gasifiers. Fuel 1991, 70, 519-527.
[85] Xiao, R.; Zhang, M. Y.; Jin, B. S.; Huang, Y.; Zhou, H. C. High-Temperature Air/Steam-Blown Gasification of Coal in a Pressurized Spout-Fluid Bed. Energy Fuels 2006, 20, 715-720.
[86] Huang, J. J.; Fang, Y. T.; Chen, H. S.; Wang, Y. Coal Gasification Characteristic in a Pressurized Fluidized Bed. Energy Fuels 2003, 17, 1474-1479.
[87] Wang, X. F.; Jin, B. S.; Zhong, W. Q. Three-dimensional simulation of fluidized bed coal gasification. Chem. Eng. Process. 2009, 48, 695-705.
[88] Hobbs, M. L.; Radulovic, P. T.; Smoot, L. D. Prediction of effluent compositions for fixed-bed coal gasifiers. Fuel 1992, 71, 1177-1194.
[89] Ocampo, A.; Arenas, E.; Chejne, F.; Espinel, J.; Londoño, C.; Aguirre, J.; Perez, J. D. An experimental study on gasification of Colombian coal in fluidised bed. Fuel 2003, 82, 161-164.
[90] Shadle, L. J.; Monazam, E. R.; Swanson, M. L. Coal Gasification in a Transport Reactor. Ind. Eng. Chem. Res. 2001, 40, 2782-2792.
[91] Elia, J. A.; Baliban, R. C.; Floudas, C. A. Toward Novel Hybrid Biomass, Coal, and Natural Gas Processes for Satisfying Current Transportation Fuel Demands, 2: Simultaneous Heat and Power Integration. Ind. Eng. Chem. Res. 2010, 49, DOI: 10.1021/ie100064q.
[92] Floudas, C. A. Nonlinear and Mixed-Integer Optimization; Oxford University Press: New York, 1995.
[93] Ciric, A. R.; Floudas, C. A. Heat exchanger network synthesis without decomposition. Comput. Chem. Eng. 1991, 15, 385-396.
[94] Ciric, A. R.; Floudas, C. A. Application of the simultaneous matchnetwork optimization approach to the pseudo-pinch problem. Comput. Chem. Eng. 1990, 14, 241-250.
[95] Ciric, A. R. Global Optimum and Retrofit Issues in Heat Exchanger Network and Utility System Synthesis, Ph.D. Thesis, Princeton University, Princeton, N.J., 1990.
[96] Yee, T. F.; Grossmann, I. E. Simultaneous optimization models for heat integrationsII. Heat exchanger network synthesis. Comput. Chem. Eng. 1990, 14, 1165-1184.
[97] Floudas, C. A.; Ciric, A. R. Strategies for overcoming uncertainties in heat exchanger network synthesis. Comput. Chem. Eng. 1989, 13, 1133-1152.
[98] Floudas, C. A.; Ciric, A. R.; Grossmann, I. E. Automatic synthesis of optimum heat exchanger network configurations. AIChE J. 1986, 32, 276-290.
[99] Holiastos, K.; Manousiouthakis, V. Minimum hot/cold/electric utility cost for heat exchange networks. Comput. Chem. Eng. 2002, 26, 3-16.
[100] Seider, W. D.; Seader, J. D.; Lewin, D. R. Product and Process Design Principles: Synthesis, Analysis, and EValuation; John Wiley and Sons: New York, 2004.
[101] Incropera, F. P.; DeWitt, D. P. Fundamentals of Heat and Mass Transfer; John Wiley and Sons: New York, 2006.
[102] Kreutz, T. G.; Larson, E. D.; Liu, G.; Williams, R. H. Fischer-Tropsch Fuels from Coal and Biomass. In Proceedings of the 25^{th} International Pittsburgh Coal Conference, 2008.
[103] N. E. T. L. Cost and Performance Baseline for Fossil Energy Plants. Vol. 1: Bituminous Coal and Natural Gas to Electricity Final Report, Document No. DOE/NETL-2007/1281, 2007 (available via the Internet at http://www.netl.doe.gov/energy-analyses/baselinetudies.html).
[104] CPLEX ILOG CPLEX C++ API 11.1 Reference Manual, 2008.
[105] National Research Council. The Hydrogen Economy: Opportunities, Costs, Barriers, and R&D Needs; The National Academies Press: Washington, D.C., 2004.
[106] Gundersen, T.; Grossmann, I. E. Improved Optimization Strategies for Automated Heat Exchanger Network Synthesis Through Physical Insights. Comput. Chem. Eng. 1990, 14, 925-944.
[107] Bechtel. Aspen Process Flowsheet Simulation Model of a Battelle Biomass-Based Gasification, Fischer-Tropsch Liquefaction and Combined-Cycle Power Plant. Contract No. DE-AC22-93PC91029, 1998 (available via the Internet at http://www.Fischer-Tropsch.org/).
[108] Bechtel. Baseline Design/Economics for Advanced Fischer-Tropsch Technology. Contract No. DE-AC22-91PC90027. Quarterly Report, April-June 1992 (http://www.Fischer-Tropsch.org/).
[109] Bechtel. Baseline Design/Economics for Advanced Fischer-Tropsch Technology. Contract No. DE-AC22-91PC90027. Quarterly Report, January-March 1993 (http://www.Fischer-Tropsch.org/).
[110] Bechtel. Baseline Design/Economics for Advanced Fischer-Tropsch Technology. Contract No. DE-AC22-91PC90027. Quarterly Report, April-June 1994 (http://www.Fischer-Tropsch.org/).
[111] Viswanathan, J.; Grossmann, I. E. A Combined Penalty Function and Outer-approximation Method for MINLP Optimization. Comput. Chem. Eng. 1990, 14, 769-782.
[112] Drud, A. CONOPT: A GRG code for large sparse dynamic nonlinear optimization problems. Math. Program. 1985, 31, 153-191.
[113] Floudas, C. A. Deterministic Global Optimization: Theory, Methods and Applications; Kluwer Academic Publishers: Dordrecht, The Netherlands, 2000.
[114] Floudas, C. A.; Akrotirianakis, I. G.; Caratzoulas, S.; Meyer, C. A.; Kallrath, J. Global optimization in the 21st century: Advances and challenges. Comput. Chem. Eng. 2005, 29, 1185-1202.
[115] Floudas, C. A.; Gounaris, C. E. A review of recent advances in global optimization. J. Global Optim. 2009, 45, 3-38.
[116] Floudas, C. A.; Pardalos, P. M. State-of-the-art in global optimization: Computational methods and applications Preface. J. Global Optim. 1995, 7, 113.
[117] Ind. Eng. Chem. Res., Vol. 49, No. 16, 2010 7387 Comparison of Piecewise-Linear Relaxations for Pooling Problems. Ind. Eng. Chem. Res. 2009, 48, 5742-5766.
[118] Misener, R.; Floudas, C. A. Advances for the Pooling Problem: Modeling, Global Optimization, and Computational Studies. Appl. Comput. Math. 2009, 8, 3-22.
[119] Adjiman, C. S.; Dallwig, S.; Floudas, C. A.; Neumaier, A. A Global Optimization Method, RBB for general twice differentiable NLPssI. Theoretical Advances. Comput. Chem. Eng. 1998, 22, 1137-1158.
[120] Adjiman, C. S.; Androulakis, I. P.; Floudas, C. A. A Global Optimization Method, RBB for general twice differentiable NLPssII. Implementation and Computational Results. Comput. Chem. Eng. 1998, 22, 1159-1179.
[121] Adjiman, C. S.; Androulakis, I. P.; Floudas, C. A. Global Optimization of Mixed Integer Nonlinear Problems. AIChE J. 2000, 46, 1769-1797.
[122] Baliban, R.; Elia, J.; Floudas, C. Toward Novel Hybrid Biomass, Coal, and Natural Gas Processes for Satisfying Current Transportation Fuel Demands, 1: Process Alternatives, Gasification Modeling, Process Simulation, and Economic Analysis. Ind. Eng. Chem. Res. 2010, 49, DOI: 10.1021/ie100063y.
[123] Adams, T. A., II, & Barton, P. I. (2010). High-efficiency power production from coal with carbon capture. AIChE Journal, 56(12), 3120-3136.
[124] Adams, T. A., II, & Barton, P. I. (2011). Combining coal gasification and natural gas reforming for efficient polygeneration. Fuel Processing Technology, 92(3), 639-655.
[125] Agrawal, R., Singh, N. R., Ribeiro, F. H., & Delgass, W. N. (2007). Sustainable fuel for the transportation sector. PNAS, 104(12), 4828-4833.
[126] Ahmetovic, E., & Grossmann, I. E. (2010a). Optimization of energy and water consumption in corn-based ethanol plants. Industrial and Engineering Chemistry Research, 49(17), 7972-7982.
[127] Ahmetovic, E., & Grossmann, I. E. (2010b). Global superstructure optimization for the design of integrated process water networks. AIChE Journal, 57(2), 434-457.
[128] Argonne National Laboratory. GREET 1.8b (2007). The greenhouse gases, regulated emissions, and energy use in transportation (GREET) model, release September 2008.
[129] Baliban, R. C., Elia, J. A., & Floudas, C. A. (2010). Toward novel biomass, coal, and natural gas processes for satisfying current transportation fuel demands, 1: Process alternatives, gasification modeling, process simulation, and economic analysis. Industrial and Engineering Chemistry Research, 49(16), 7343-7370.
[130] Baliban, R. C., Elia, J. A., & Floudas, C. A. (2011). Optimization framework for the simultaneous process synthesis, heat and power integration of a thermochemical hybrid biomass, coal, and natural gas facility. Computers and Chemical Engineering, 35(9), 1647-1690.
[131] Baliban, R. C., Elia, J. A., & Floudas, C. A. (2012). Simultaneous process synthesis, heat, power, and water integration of thermochemical hybrid biomass, coal, and natural gas facilities. Computers and Chemical Engineering, 37(10), 297-327.
[132] Baliban, R. C., Elia, J. A., Misener, R., & Floudas, C. A. (2012). Global optimization of a MINLP process synthesis model for thermochemical based conversion of hybrid coal, biomass, and natural gas to liquid fuels. Computers and Chemical Engineering, 42, 64-86.
[133] Bechtel (1992). Baseline design/economics for advanced Fischer-Tropsch technology. Contract No. DE-AC22-91PC90027.
[134] Bechtel (1998). Aspen process flowsheet simulation model of a battelle biomass-based gasification, Fischer-Tropsch liquefaction and combined-cycle power plant. Contract number: DE-AC22-93PC91029, http://www.fischertropsch.org/.
[135] Bechtel Corp, Global Energy Inc., and Nexant Inc. (2003). Gasification plant cost and performance optimization, task 2 topical report: Coke/coal gasification with Liquids Coproduction. USDOE contract DE-AC26-99FT40342.
[136] Cao, Y., Gao, Z., Jin, J., Zhou, H., Cohron, M., Zhao, H., et al. (2008). Synthesis gas production with an adjustable H2/CO ratio through the coal gasification process: Effects of coal ranks and methane addition. Energy and Fuels, 22(3), 1720-1730.
[137] Chen, Y., Adams, T. A., II, & Barton, P. I. (2011a). Optimal design and operation of static energy polygeneration systems. Industrial and Engineering Chemistry Research, 50(9), 5099-5113.
[138] Chen, Y., Adams, T. A., II, & Barton, P. I. (2011b). Optimal design and operation of flexible energy polygeneration systems. Industrial and Engineering Chemistry Research, 50(8), 4553-4566.
[139] Chiesa, P., Consonni, S., Kreutz, T., & Williams, R. (2005). Co-production of hydrogen, electricity and CO2 from coal with commercially ready technology. Part A: Performance and emissions. International Journal of Hydrogen Energy, 30(7), 747-767. CPLEX ILOG CPLEX C++ API 12.1 Reference Manual, 2009.
[140] de Fraiture, C., Giordano, M., & Liao, Y. (2008). Biofuels and implications for agricultural water use: Blue impacts of green energy. Water Policy, 10, 67-81. de Klerk, A. (2011). Fischer-Tropsch refining. Wiley-VCH Verlag & Co. KGaA.
[141] Department of Energy (2005). Biomass as feedstock for a bioenergy and bioproducts industry: The technical feasibility of a billion ton annual supply. Document number: DOE/GO-102005-2135.http://www1.eere.energy.gov/biomass/publications.html.
[142] Duran, M. A., & Grossmann, I. E. (1986). Simultaneous optimization and heat integration of chemical processes. AIChE Journal, 32(1), 123-138.
[143] Elia, J. A., Baliban, R. C., & Floudas, C. A. (2010). Toward novel biomass, coal, and natural gas processes for satisfying current transportation fuel demands, 2: Simultaneous heat and power integration. Industrial and Engineering Chemistry Research, 49(16), 7371-7388.
[144] Elia, J. A., Baliban, R. C., Xiao, X., & Floudas, C. A. (2011). Optimal energy supply network determination and life cycle analysis for hybrid coal, biomass, and natural gas to liquid (CBGTL) plants using carbon-based hydrogen production. Computers and Chemical Engineering, 35(8), 1399-1430.
[145] Elia, J. A., Baliban, R. C., & Floudas, C. A. (2012). Nationwide energy supply chain analysis for hybrid feedstock processes with significant CO2 emissions reduction. AIChE Journal, 58(7), 2142-2154.
[146] Energy Information Administration (2009). Ranking of U.S. Refineries. http://www.eia.doe.gov/neic/rankings/refineries.htm.
[147] Energy Information Administration (2011). Annual Energy Outlook 2011 with Projections to 2035. Document number: DOE/EIA-0383 (2011). http://www.eta.doe.gov/oiaf/aeo/.
[148] Floudas, C. A., Akrotirianakis, I. G., Caratzoulas, S., Meyer, C. A., & Kallrath, J. (2005). Global optimization in the 21st century: Advances and challenges. Computers and Chemical Engineering, 29(6), 1185-1202.
[149] Floudas, C. A. (1995). Nonlinear and mixed-integer optimization. New York: Oxford University Press.
[150] Floudas, C. A. (2000). Deterministic global optimization: Theory, methods and applications. Dordrecht, Netherlands: Kluwer Academic Publishers.
[151] Floudas, C. A., Ciric, A. R., & Grossmann, I. E. (1986). Automatic synthesis of optimum heat exchanger network configurations. AIChE Journal, 32(2), 276-290.
[152] Floudas, C. A., Elia, J. A., & Baliban, R. C. (2012). Hybrid and Single Feedstock Energy Processes for Liquid Transportation Fuels: A Critical Review. Computers and Chemical Engineering, 41, 24-51.
[153] Floudas, C. A., & Gounaris, C. E. (2009). A review of recent advances in global optimization. Journal of Global Optimization, 45(1), 3-38.
[154] Floudas, C. A., & Pardalos, P. M. (1995). State of the art in global optimization: Computational methods and applications. Journal of Global Optimization, 7(2),113.
[155] Grossmann, I. E., & Martin, M. (2010). Energy and water optimization in biofuel plants. Chinese Journal of Chemical Engineering, 18(6), 914-922.
[156] Karuppiah, R., & Grossmann, I. E. (2006). Global optimization for the synthesis of integrated water systems in chemical processes. Computers and Chemical Engineering, 30(4), 650-673.
[157] Keil, F. J. (1999). Methanol-to-hydrocarbons: Process technology. Microporous and Mesoporpus Materials, 29(1-2), 49-66.
[158] Kreutz, T., Williams, R., Consonni, S., & Chiesa, P. (2005). Co-production of hydrogen, electricity and CO2 from coal with commercially ready technology. Part b: Economic analysis. International Journal of Hydrogen Energy, 30(7), 769-784.
[159] Kreutz, T. G., Larson, E. D., Liu, G., & Williams, R. H. (2008). Fischer-Tropsch fuels from coal and biomass. In Proceedings of the 25th international Pittsburgh coal conference
[160] Larson, E. D., & Jin, H. (1999). Biomass conversion to Fischer-Tropsch liquids: Preliminary energy balances. In Proceedings of the 4th biomass conference of the Americas (pp. 843-853).
[161] Larson, E. D., Jin, H., & Celik, F. E. (2009). Large-scale gasification-based coproduction of fuels and electricity from switchgrass. Biofuels, Bioproducts and Biorefining, 3, 174-194.
[162] Liu, P., Pistikopoulos, E. N., & Li, Z. (2009). A mixed-integer optimization approach for polygeneration energy systems design. Computers and Chemical Engineering, 33(3), 759-768.
[163] Liu, P., Pistikopoulos, E. N., & Li, Z. (2010a). Decomposition based stochastic programming approach for polygeneration energy systems design under uncertainty. Industrial and Engineering Chemistry Research, 49(7), 3295-3305.
[164] Liu, P., Pistikopoulos, E. N., & Li, Z. (2010b). A multi-objective optimization approach to polygeneration energy systems design. AIChE Journal, 56(5), 1218-1234.
[165] Lynd, L. R., Larson, E., Greene, N., Laser, M., Sheehan, J., Dale, B. E., et al. (2009). The role of biomass in America's energy future: Framing the analysis. Biofuels, Bioproducts and Biorefining, 3(2), 113-123.
[166] Martin, M., & Grossmann, I. E. (2011). Process Optimization of FT-Diesel Production from Lignocellulosic Switch grass. Industrial and Engineering Chemistry Research, 50(23), 13485-13499.
[167] Mobil Research and Development Corporation (1978). Research Guidance Studies to Assess gasoline from coal by methanol-to-gasoline and Sasol-type Fischer-Tropsch technologies. USDOE contract EF-77-C-O1-2447.
[168] Mobil Research and Development Corporation (1986). Slurry Fischer-Tropsch/mobil two stage process of converting syngas to high octane gasoline. USDOE contract DE-AC22-80PC30022.
[169] Mobil Research and Development Corporation (1985). Two-stage process for conversion of synthesis gas to high quality transpiration fuels. USDOE contract DE-AC22-83PC60019.
[170] Nashawi, I. S., Malallah, A., & Al-Bisharah, M. (2010). Forecasting world crude oil production using multicyclic Hubbert model. Energy and Fuels, 24(3), 1788-1800.
[171] National Academy of Sciences. (2009). National Academy of Engineering, and National Research Council. Liquid transportation fuels from coal and biomass: Technological status, costs, and environmental issues. Washington, D.C.: EPA.
[172] National Energy Technology Laboratory (2007). Cost and performance baseline for fossil energy plants. Volume 1: Bituminous coal and natural gas to electricity final report. Document number: DOE/NETL-2007/1281. http://www.netl.doe.gov/energy-analyses/baseline studies.html.
[173] National Renewable Energy Laboratory (2011). Gasoline from Wood via integrated gasification, synthesis, and methanol-to-gasoline technologies. USDOE contract DE-AC36-08GO28308.
[174] National Research Council (2008). Water implications of biofuels production in the United States. http://www.nap/edu/catalog/12039.html.
[175] Spath, P., Aden, A., Eggeman, T., Ringer, M., Wallace, B., & Jechura, J. Biomass to hydrogen production detailed design and economics utilizing the battelle columbus laboratory indirectly-heated gasifier. Document number: NREL/TP-510-37408. http://www.nrel.gov/docs/fy05osti/37408.pdf.
[176] Sudiro, M., & Bertucco, A. (2007). Synthetic fuels by a limited CO2 emission process which uses both fossil and solar energy. Energy and Fuels, 21, 3668-3675.
[177] Sudiro, M., & Bertucco, A. (2009). Production of synthetic gasoline and diesel fuel by alternative processes using natural gas and coal: Process simulation and optimization. Energy, 34, 2206-2214.
[178] Sudiro, M., Bertucco, A., Ruggeri, F., & Fontana, M. (2008). Improving process performances in coal gasification for power and synfuel production. Energy and Fuels, 22(6), 3894-3901.
[179] Tabak, S. A., & Krambeck, F. J. (1985). Shaping process makes fuels. Hydrocarbon Processing, 64(9), 72-74.
[180] Tabak, S. A., Krambeck, F. J., & Garwood, W. E. (1986). Conversion of propylene and butylene over ZSM-5 catalyst. AIChE Journal, 32(9), 1526-1531.
[181] Tabak, S. A., & Yurchak, S. (1990). Conversion of methanol over ZSM-5 to fuels and chemicals. Catalysis Today, 6(3), 307-327.
[182] Toyir, J., Miloua, R., Elkadri, N. E., Nawdali, M., Toufik, H., Miloua, F., et al. (2009). Sustainable process for the production of methanol from CO2 and H2 using Cu/ZnO-based multicomponent catalyst. Physics Procedia, 2(3), 1075-1079.
[183] US Government Printing Office (2009). Economic indicators. http://www.gpoaccess.giv/indicators/index.html.
[184] Vliet, O., Faaij, A., & Turkenburg, W. (2009). Fischer-Tropsch diesel production in a well-wheel perspective: A carbon, energy flow and cost analysis. Energy Conversion and Management, 50(4), 855-876.
[185] Water Science and Technology Board (2008). Water Implications of Biofuels Production in the United States.
[186] Weekman, V. W., Jr. (2010). Gazing into an energy crystal ball. Chemical Engineering Progress, 6, 23-27.
[187] Zittel, W., Schindler, J. (2007). Crude oil: The supply outlook. Document number: EWG-series No. 3/2007. Report to the Energy Watch Group, October 2007.
[188] R. C. Baliban, J. A. Elia, and C. A. Floudas. Optimization Framework for the Simultaneous Process Synthesis, Heat and Power Integration of a Thermochemical Hybrid Biomass, Coal, and Natural Gas Facility. Comp. Chem. Eng., 35(9):1647-1690, 2011.
[189] R. C. Baliban, J. A. Elia, and C. A. Floudas. Simultaneous Process Synthesis, Heat, Power, and Water Integration of Thermochemical Hybrid Biomass, Coal, and Natural Gas Facilities. Comp. Chem. Eng., 37(10):297-327, 2012.
[190] R. C. Baliban, J. A. Elia, and C. A. Floudas. Toward Novel Biomass, Coal, and Natural Gas Processes for Satisfying Current Transportation Fuel Demands, 1: Process Alternatives, Gasification Modeling, Process Simulation, and Economic Analysis. Ind. Eng. Chem. Res., 49(16):7343-7370, 2010.
[191] T. G. Kreutz, E. D. Larson, G. Liu, and R. H. Williams. Fischer-Tropsch Fuels from Coal and Biomass. Proceedings of the 25th International Pittsburgh Coal Conference, 2008.
[192] National Academy of Sciences, National Academy of Engineering, and National Research Council. Liquid Transportation Fuels from Coal and Biomass: Technological Status, Costs, and Environmental Issues. Washington, D.C., EPA, 2009.
[193] Bechtel. Aspen Process Flowsheet Simulation Model of a Battelle Biomass-Based Gasification, Fischer-Tropsch Liquefaction and Combined-Cycle Power Plant. Contract Number: DE-AC22-93PC91029. http://www.Fischer-Tropsch.org/, 1998.
[194] Bechtel. Baseline design/economics for advanced Fischer-Tropsch technology. Contract No. DE-AC22-91PC90027, 1992.
[195] M. Dry. The Fischer-Tropsch process:1950-2000. Catalysis Today, 71(3-4):227-241, 2002.
[196] R. W. R. Zwart and H. Boerrigter. High efficiency co-production of Synthetic Natural Gas (SNG) and Fischer-Tropsch (FT) transportation fuels from biomass. Energy and Fuels, 19(2):591-597, 2005.
[197] R. Oukaci. Fischer-Tropsch synthesis, 2002. 2nd Annual Global GTL Summit Executive Briefing, May 28-30, 2002, London, U.K.
[198] Mobil Research and Development Corporation. Slurry Fischer-Tropsch/Mobil Two Stage Process of Converting Syngas to High Octane Gasoline. USDOE contract DE-AC22-80PC30022, 1983.
[199] Mobil Research and Development Corporation. Two-Stage Process For Conversion of Synthesis Gas to High Quality Transportation Fuels. USDOE contract DE-AC22-83PC60019, 1985.
[200] Bechtel. Baseline Design/Economics for Advanced Fischer-Tropsch Technology. Quarterly Report, January-March 1993. Contract No. DE-AC22-91PC90027, 1993.
[201] Mobil Research and Development Corporation. Research Guidance Studies to Assess Gasoline from Coal by Methanol-To-Gasoline and Sasol-Type Fischer-Tropsch Technologies. USDOE contract EF-77-C-O1-2447, 1978.
[202] S. A. Tabak and F. J. Krambeck. Shaping Process Makes Fuels. Hydrocarbon Processing, 64(9):72-74, 1985.
[203] S. A. Tabak, F. J. Krambeck, and W. E. Garwood. Conversion of propylene and butylene over ZSM-5 catalyst. AIChE J., 32(9):1526-1531, 1986.
[204] S. A. Tabak and S. Yurchak. Conversion of methanol over ZSM-5 to fuels and chemicals. Catalysis Today, 6(3):307-327, 1990.
[205] F. J. Keil. Methanol-to-hydrocarbons: process technology. Microporous and Mesoporpus Materials, 29(1-2):49-66, 1999.
[206] National Renewable Energy Laboratory. Gasoline from Wood via Integrated Gasification, Synthesis, and Methanol-to-Gasoline Technologies. USDOE contract DE-AC36-08GO28308, 2011.
[207] National Energy Technology Laboratory. Quality Guidelines for Energy System Studies, 2004.
[208] A. van der Drift and J. van Doorn. Analysis of biomass data in ECN database Phyllis. http://www.ecn.nl/phyllis/, 2002.
[209] Energy Information Administration. Monthly Energy Review—May 2012. Document Number: DOE-EIA-0035(2012/05), 2012. Available at: http://www.eta.doe.gov/mer/. Accessed August 2012
[210] Energy Information Administration. Annual Energy Outlook 2013 with Projections to 2035. Document Number: DOE/EIA-0383 (2012), 2011. Available at: http://www.eta.doe.gov/oiaf/aeo/. Accessed August 2012.
[211] Floudas C A, Elia J A, Baliban R C. Hybrid and single feedstock energy processes for liquid transportation fuels: a critical review. Comp Chem. Eng. 2012; 41:24-51.
[212] Lynd L R, Larson E, Greene N, Laser M, Sheehan J, Dale B E, McLaughlin S, Wang M. The role of biomass in America's energy future: framing the analysis. Biofuels Bioprod Biorefin. 2009; 3(2):113-123.
[213] Department of Energy. Biomass as Feedstock for a Bioenergy and Bioproducts Industry: The Technical Feasibility of a Billion-Ton Annual Supply. Document Number: DOE/GO-102005-2135.2005. Available at: http://www1.eere.energyogov/biomass/publications.html. Accessed August 2012.
[214] National Research Council. Water Implications of Biofuels Production in the United States. National Research Council, 2008. Available at: http://www.nap/edu/catalog/12039.html. The National Academies Press, Washington, D.C. Last Accessed August 2012.
[215] de Fraiture C, Giordano M, Liao Y. Biofuels and implications for agricultural water use: blue impacts of green energy. Water Policy. 2008; 10:67-81.
[216] Kreutz T G, Larson E D, Liu G, Williams R H. Fischer-Tropsch fuels from coal and biomass. In: Proceedings of the 25th International Pittsburgh Coal Conference, 2008. Pittsburgh, Pa., 29 Sep.-2 Oct., 2008.
[217] de Klerk A. Fischer-Tropsch Refining. Weinheim, Germany: Wiley-VCH, 2011.
[218] National Academy of Sciences, National Academy of Engineering, and National Research Council. Liquid Transportation Fuels from Coal and Biomass: Technological Status, Costs, and Environmental Issues. Prepublication. Washington, D.C.: EPA, 2009.
[219] Agrawal R, Singh N R, Ribeiro F H, Delgass W N. Sustainable fuel for the transportation sector. Proc Natl Acad Sci USA. 2007; 104:4828-4833.
[220] Baliban R C, Elia J A, Floudas C A. Toward novel biomass, coal, and natural gas processes for satisfying current transportation fuel demands. 1. Process alternatives, gasification modeling, process simulation, and economic analysis. Ind Eng Chem. Res. 2010; 49:7343-7370.
[221] Elia J A, Baliban R C, Floudas C A. Toward novel biomass, coal, and natural gas processes for satisfying current transportation fuel demands. 2. Simultaneous heat and power integration. Ind Eng Chem. Res. 2010; 49:7371-7388.
[222] Baliban R C, Elia J A, Floudas C A. Optimization framework for the simultaneous process synthesis, heat and power integration of a thermochemical hybrid biomass, coal, and natural gas facility. Comp Chem. Eng. 2011; 35:1647-1690.
[223] Baliban R C, Elia J A, Floudas C A. Simultaneous process synthesis, heat, power, and water integration of thermochemical hybrid biomass, coal, and natural gas facilities. Comp Chem. Eng. 2012; 37:297-327.
[224] Peng X D, Wang A W, Toseland B A, Tij P J A. Single-step syngas-to-dimethyl ether processes for optimal productivity, minimal emissions, and natural gas-derived syngas. Ind Eng Chem. Res. 1999; 38:4381-4388.
[225] Sudiro M, Bertucco A. Synthetic fuels by a limited CO2 emission process which uses both fossil and solar energy. Energy Fuels. 2007; 21:3668-3675.
[226] Cao Y, Gao Z, Jin J, Zhou H, Cohron M, Zhao H, Liu H, Pan W. Synthesis gas production with an adjustable H2/CO ratio through the coal gasification process: effects of coal ranks and methane addition. Energy Fuels. 2008; 22(3):1720-1730.
[227] Zhou L, Hu S, Li Y, Zhou Q. Study on co-feed and co-production system based on coal and natural gas for producing dme and electricity. hem Eng J. 2008; 136:31-40.
[228] Sudiro M, Bertucco A. Production of synthetic gasoline and diesel fuel by alternative processes using natural gas and coal: process simulation nd optimization. Energy. 2009; 34:2206-2214.
[229] Zhou L, Hu S, Chen D, Li Y, Zhu B, Jin Y. Study on systems based on coal and natural gas for producing dimethyl ether. Ind Eng Chem es. 2009; 48:4101-4108.
[230] Adams T A II, Barton P I. Combining coal gasification and natural gas reforming for efficient polygeneration. Fuel Proc Technol. 2011; 92:639-655.
[231] Adams T A II, Barton P I. Combining coal gasification, natural gas reforming, and solid oxide fuel cells for efficient polygeneration with CO2 capture nd sequestration. Fuel Proc Technol. 2011; 92(10):2105-2115.
[232] Li Z, Liu P, He F, Wang M, Pistikopoulos E N. Simulation and exergoeconomic analysis of a dual-gas sourced polygeneration process with integrated methanol/DME/DMC catalytic synthesis. Comp Chem. Eng. 2011; 35(9):1857-1862.
[233] Borgwardt R H. Biomass and natural gas as co-feedstocks for production of fuel for fuel-cell vehicles. Biomass Bioenergy. 1997; 12(5):333-345.
[234] Dong Y, Steinberg M. Hynol—an economical process for methanol production from biomass and natural gas with reduced CO2 emission. Int J Hydrogen Energy. 1997; 22(10-11):971-977.
[235] Li H, Hong H, Jin H, Cai R. Analysis of a feasible polygeneration system for power and methanol production taking natural gas and biomass as materials. Appl Energy. 2010; 87:2846-2853.
[236] Liu G, Williams R H, Larson E D, Kreutz T G. Design/economics of low-carbon power generation from natural gas and biomass with synthetic fuels co-production. Energy Procedia. 2011; 4:1989-1996.
[237] Baliban R C, Elia J A, Misener R, Floudas C A. Global optimization of a MINLP process synthesis model for thermochemical based conversion of hybrid coal, biomass, and natural gas to liquid fuels. Comp Chem. Eng. 2012; 42; 64-86.
[238] Baliban R C, Elia J A, Weekman V W, Floudas C A. Process synthesis of hybrid coal, biomass, and natural gas to liquids via Fischer Tropsch synthesis, ZSM-5 catalytic conversion, methanol synthesis, methanol-to-gasoline, and methanol-to-olefins/distillate technologies. Comp Chem. Eng. 2012; 47:29-56.
[239] Fox J M, Chen T P, Degen B D. Direct Methane Conversion Process Evaluations. Contract No. DE-AC22-87PC79814, 1988.
[240] Gradassi M J, Green N R. Economics of natural gas conversion processes. Fuel Proc Technol. 1995; 42:65-83.
[241] Iandoli C L, Kjelstrup S. Exergy analysis of a GTL process based on low-temperature slurry F-T reactor technology with a cobalt catalyst. Energy Fuels. 2007; 21:2317-2324.
[242] Gao L, Li H, Chen B, Jin H, Lin R, Hong H. Proposal of a natural gas-based polygeneration system for power and methanol production. Energy. 2008; 33:206-212.
[243] Hao X, Djatmiko M E, Xu Y, Wang Y, Chang J, Li Y. Simulation analysis of a gas-to-liquid process using Aspen Plus. Chem Eng Technol. 2008; 31(2):188-196.
[244] Lee C J, Lim Y, Kim H S, Han C. Optimal gas-to-liquid product selection from natural gas under uncertain price scenarios. Ind Eng Chem. Res. 2009; 48:794-800.
[245] Kim Y H, Jun K W, Joo H, Han C, Song I K. A simulation study on gas-to-liquid (natural gas to Fischer-Tropsch synthetic fuel) process optimization. Chem Eng J. 2009; 155:427-432.
[246] Bao B, El-Halwagi M M, Elbashir N O. Simulation, integration, and economic analysis of gas-to-liquid process. Fuel Proc Technol. 2010; 91:703-713.
[247] Dillerop C, vander Berg H, vander Ham A G J. Novel syngas production techniques for GTL-FT synthesis of gasoline using reverse flow catalytic membrane reactors. Ind Eng Chem. Res. 2010; 49:12529-12537,
[248] Ha K S, Bae J W, Woo K J, Jun K W. Efficient utilization of greenhouse gas in a gas-to-liquids process combined with carbon dioxide reforming of methane. Environ Sci Technol. 2010; 44:1412-1417.
[249] Heimel S, Lowe C. Technology comparison of CO2 capture for a gas-to-liquids plant. Energy Procedia. 2009; 1:4039-4046.
[250] Bin C, Hingguang J, Lin G. System study on natural gas-based polygeneration system of DME and electricity. Int J Energy Res. 2008; 32:722-734.
[251] Hall K R. A new gas to liquids (GTL) or gas to ethylene (GTE) technology. Catal Today. 2005; 106:243-246.
[252] Suzuki S, Sasaki T, Kojima T. New process development of natural gas conversion technology to liquid fuels via OCM reaction. Energy Fuels. 1996; 10:531-536.
[253] Horstman D, Abata D, Keith J, Oberto L. Feasibility study of an onboard natural gas to dimethyl ether reactor for dimethyl ether preinjection and enjanced ignition. J Eng Gas Turbines Power. 2005; 127:909-917.
[254] Erturk, M. Economic analysis of unconventional liquid fuel sources. Renew Sustain Energy Rev. 2011; 15:2766-2771.
[255] Vliet O, Faaij A, Turkenburg W. Fischer-Tropsch diesel production in a well-wheel perspective: a carbon, energy flow and cost analysis. Energy Convers Manage. 2009; 50(4):855-876.
[256] Martin M, Grossmann I E. Process optimization of FT-diesel production from lignocellulosic switchgrass. Ind Eng Chem. Res. 2011; 50:13485-13499.
[257] Ellepola J, Thijssen N, Grievink J, Baak G, Avhale A, vanSchijndel J. Development of a synthesis tool for gas-to-liquid complexes. Comp Chem. Eng. 2012; 42:2-14.
[258] Duran M A, Grossmann I E. Simultaneous optimization and heat integration of chemical processes. AIChE J. 1986; 32(1):123-138.
[259] Karuppiah R, Grossmann I E. Global optimization for the synthesis of integrated water systems in chemical processes. Comp Chem Eng. 2006; 30(4):650-673.
[260] Ahmetovic E, Grossmann I E. Optimization of energy and water consumption in corn-based ethanol plants. Ind Eng Chem. Res. 2010; 49(17):7972-7982.
[261] Grossmann I E, Martin M. Energy and water optimization in biofuel plants. Chin J Chem. Eng. 2010; 18(6):914-922.
[262] Ahmetovic E, Grossmann I E. Global superstructure optimization for the design of integrated process water networks. AIChE J. 2010; 57(2):434-457.
[263] National Energy Technology Laboratory. Quality Guidelines for Energy System Studies. National Energy Technology Laboratory, 2004.
[264] National Energy Technology Laboratory. Assessment of Hydrogen Production with CO2 Capture Volume 1: Baseline State-of-the-Art Plants. Document Number: DOE/NETL-2010/1434, 2010.
[265] Zhang Q, He D, Li J, Xu B, Liang Y, Zhu Q. Comparatively high yield methanol production from gas phase partial oxidation of methane. Appl Catal A. 2002; 224:201-207.
[266] Zhang Q, He D, Zhu Q. Recent progress in direct partial oxidation of methane to methanol. J Nat Gas Chem. 2003; 12:81-89.
[267] Rasmussen C L, Glarborg P. Direct partial oxidation of natural gas to liquid chemicals: chemical kinetic modeling and global optimization. Ind Eng Chem. Res. 2008; 47(17):6579-6588.
[268] Keller G E, Bhasin M M. Synthesis of ethylene via oxidative coupling of methane. I. Determination of active catalysts. J Catal. 1992; 73(1):9-19.
[269] Jones A C, Leonard J J, Sofranko J A. The oxidative conversion of methane to higher hydrocarbons over alkali-promoted Mn/SiO2. J Catal. 1987; 103:311-319.
[270] Lee J S, Oyama S T. Oxidative coupling of methane to higher hydrocarbons. Catal Rev Sci Eng. 1988; 30(2):249-280.
[271] Jones A C, Leonard J J, Sofranko J A. The oxidative conversion of methane to higher hydrocarbons over alkali-promoted Mn/SiO₂. U.S. Pat. Nos. 4,443,644, 4,443,645, 4,443,646, 4,443,647, 4,443,648, 4,443,649, 4,444,984 (1984); 4,448,322, 4,499,323, 4,523,049, 4,523,050, 4,544,784, 4,560,821 (1985); 4,567,307 (1986).
[272] Fox J M III, Chen T P, Degen B D. An evaluation of direct methane conversion processes. Chem Eng Prog. 1990; 86:42-50.
[273] Lunsford J H. The catalytic oxidative coupling of methane. Agnew Chem Int Ed Engl. 1995; 34:970-980.
[274] Hall K R, Holtzapple M T, Capareda S C. Integrated biofuel production system. U.S. Pat. No. 8,153,850 (2012).
[275] Hall K R, Bullin J A, Eubank P T, Akgerman A, Anthony R G. Method for converting natural gas to olefins. U.S. Pat. No. 6,130,260 (2000); U.S. Pat. No. 6,323,247 (2001); U.S. Pat. No. 6,433,235 (2002); U.S. Pat. No. 6,602,920 (2003); U.S. Pat. Nos. 7,045,670, 7,119,240 (2006); U.S. Pat. Nos. 7,183,451, 7,208,647, 7,250,449 (2007); U.S. Pat. No. 7,408,091 (2008).
[276] Mobil Research and Development Corporation. Slurry Fischer-Tropsch/Mobil Two Stage Process of Converting Syngas to High Octane Gasoline. USDOE contract DE-AC22-80PC30022, 1983.
[277] Mobil Research and Development Corporation. Two-Stage Process for Conversion of Synthesis Gas to High Quality Transportation Fuels. USDOE contract DE-AC22-83PC60019, 1985.
[278] Bechtel. Aspen Process Flowsheet Simulation Model of a Battelle Biomass-Based Gasification, Fischer-Tropsch Liquefaction and Combined-Cycle Power Plant. Contract Number: DE-AC22-93PC91029, 1998. Available at: http://www.Fischer-Tropsch.org/. Accessed August 2012.
[279] Bechtel. Baseline design/economics for advanced Fischer-Tropsch technology. Contract No. DE-AC22-91PC90027, 1992.
[280] National Renewable Energy Laboratory. Gasoline from Wood via Integrated Gasification, Synthesis, and Methanol-to-Gasoline Technologies. USDOE contract DE-AC36-08GO28308, 2011.
[281] Mobil Research and Development Corporation. Research Guidance Studies to Assess Gasoline from Coal by Methanol-To-Gasoline and Sasol-Type Fischer-Tropsch Technologies. USDOE contract EF-77-C-O1-2447, 1978.
[282] Tabak S A, Yurchak S. Conversion of methanol over ZSM-5 to fuels and chemicals. Catal Today. 1990; 6(3):307-327.
[283] National Energy Technology Laboratory. Cost and Performance Baseline for Fossil Energy Plants. Vol. 1: Bituminous Coal and Natural Gas to Electricity Final Report. Document Number: DOE/NETL-2007/1281, 2007. Available at: http://www.netl.doe.gov/energy-analyses/baseline_studies.html. Accessed August 2012.
[284] Chemical Engineering Magazine. Chemical Engineering Plant Cost Index, 2012. Available at: http://www.che.com/pci/ Accessed August 2012.
[285] CPLEX. ILOG CPLEX C11 API 12.1 Reference Manual, IBM Corporation, 2009.
[286] Drud A. Conopt: a GRG code for large sparse dynamic nonlinear optimization problems. Math Program. 1985; 31(2):153-191.
[287] Floudas C A. Nonlinear and Mixed-Integer Optimization. New York: Oxford University Press, 1995.
[288] Floudas C A. Deterministic Global Optimization: Theory, Methods and Applications. Dordrecht, The Netherlands: Kluwer Academic Publishers, 2000.
[289] Floudas C A, Pardalos P M. State of the art in global optimization: computational methods and applications. J Global Optim. 1995; 7(2):113.
[290] Floudas C A, Akrotirianakis I G, Caratzoulas S, Meyer C A, Kallrath J. Global optimization in the 21st century: advances and challenges. Comp Chem. Eng. 2005; 29(6):1185-1202.
[291] Floudas C A, Gounaris C E. A review of recent advances in global optimization. J Global Optim. 2009; 45(1):3-38.
[292] Energy Information Administration. Annual Energy Outlook 2011 with Projections to 2035. Document Number: DOE/EIA-0383 (2011), 2011. Available at: http://www.eta.doe.gov/oiaf/aeo/. Accessed August 2012.
[293] Floudas C A, Ciric A R, Grossmann I E. Automatic synthesis of optimum heat exchanger network configurations. AIChE J 1986; 32(2):276-290.
[294] Gregor J H, Gosling C D, Fullerton H E. Upgrading Fischer-Tropsch LPG with the Cyclar Process. Contract No. DE-AC22-86PC90014, 1989.
[295] Argonne National Laboratory. GREET 1.8b, The Greenhouse Gases, Regulated Emissions, and Energy Use in Transportation (GREET) Model. Argonne National Laboratory, Argonne, Ill. 2007; Released September 2008.
[296] Mobil Research and Development Corporation. Two-Stage Process For Conversion of Synthesis Gas to High Quality Transportation Fuels. USDOE contract DE-AC22-83PC60019, 1985.
[297] Mobil Research and Development Corporation. Slurry Fischer-Tropsch/Mobil Two Stage Process of Converting Syngas to High Octane Gasoline. USDOE contract DE-AC22-80PC30022, 1983.
[298] Mobil Research and Development Corporation. Research Guidance Studies to Assess Gasoline from Coal by Methanol-To-Gasoline and Sasol-Type Fischer-Tropsch Technologies. USDOE contract EF-77-C-O1-2447, 1978.
[299] Bechtel. Aspen Process Flowsheet Simulation Model of a Battelle Biomass-Based Gasification, Fischer-Tropsch Liquefaction and Combined-Cycle Power Plant. Contract Number:DE-AC22-93PC91029. http://www.Fischer-Tropsch.org/, 1998.
[300] J. M. Fox, III, T.-P. Chen, and B. D. Degen. An Evaluation of Direct Methane Conversion Processes. Chemical Eng. Progress, pages 42-50, 1990.
[301] E. D. Larson, H. Jin, and F. E. Celik. Large-scale gasification-based coproduction of fuels and electricity from switchgrass. Biofuels, Bioprod. Bioref., 3:174-194, 2009.
[302] T. G. Kreutz, E. D. Larson, G. Liu, and R. H. Williams. Fischer-Tropsch Fuels from Coal and Biomass. Proceedings of the 25th International Pittsburgh Coal Conference, 2008.
[303] National Energy Technology Laboratory. Cost and Performance Baseline for Fossil EnergyPlants. Volume 1: Bituminous Coal and Natural Gas to Electricity Final Report. Document Number: DOE/NETL-2007/1281. http://www.netl.doe.gov/energy-analyses/baseline studies.html, 2007.
[304] National Renewable Energy Laboratory. Gasoline from Wood via Integrated Gasification, Synthesis, and Methanol-to-Gasoline Technologies. USDOE contract DE-AC36-08GO28308, 2011.
[305] P. Balmer and B. Mattsson. Wastewater treatment plant operation costs. Wat. Sci. Tech., 30(4):7-15, 1994.
[306] Audet, C., Hansen, P., Jaumard, B., & Savard, G. (2000). A branch and cut algorithm for nonconvex quadratically constrained quadratic programming. Mathematical Programming, 87(1), 131-152.
[307] Cafieri, S., Lee, J., & Liberti, L. (2010). On convex relaxations of quadrilinear terms. Journal of Global Optimization, 47(4), 661-685.
[308] Keha, A. B., de Farias, I. R., Jr., & Nemhauser, G. L. (2004). Models for representing piecewise linear cost functions. Operations Research Letters, 32(1), 44-48.
[309] Kreutz, T. G., Larson, E. D., Liu, G., & Williams, R. H. (2008). Fischer-Tropsch fuels from coal and biomass. In Proceedings of the 25th International Pittsburgh Coal Conference
[310] McCormick, G. P. (1769). Computability of global solutions to factorable nonconvex programs. Part 1. Convex underestimating problems. Mathematical Programming, 10(1), 147-175.
[311] Tawarmalani, M., & Sahinidis, N. V. (2002). Convexification and global optimization in continuous and mixed-integer nonlinear programming: Theory, applications, software and applications. Norwell, Mass.: Kluwer Academic Publishers.
[312] Vielma, J. P., Ahmed, S., & Nemhauser, G. (2011). Mixed-integer models for nonseparable piecewise-linear optimization: Unifying framework and extensions. Computers and Chemical Engineering, 58(2), 303-315.
[313] Vliet, O., Faaij, A., & Turkenburg, W. (2009). Fischer-Tropsch diesel production in a well-wheel perspective: A carbon, energy flow and cost analysis. Energy Conversion and Management, 50(4), 855-876.
[314] Vielma, J. P., & Nemhauser, G. (2011). Modeling disjunctive constraints with a logarithmic number of binary variables and constraints. Computers and Chemical Engineering, 128(1-2), 49-72.
[315] Wicaksono, D. S., & Karimi, I. A. (2008). Piecewise MILP under- and overestimators for global optimization of bilinear programs. AIChE Journal, 54(4), 991-1008.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

Any single embodiment herein may be supplemented with one or more element from any one or more other embodiment herein.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A superstructure for a refinery comprising:
at least one synthesis gas production unit configured to produce at least one synthesis gas selected from the group consisting of a biomass synthesis gas production unit, a coal synthesis gas production unit and a natural gas synthesis gas production unit, wherein the selection of the at least one synthesis gas production unit is determined by a mixed-integer linear optimization model solved by a global optimization framework;
a synthesis gas cleanup unit configured to remove undesired gases from the at least one synthesis gas;
a liquid fuels production unit selected from the group consisting of a Fischer-Tropsch unit and a methanol synthesis unit, the Fischer-Tropsch unit being configured to produce a first output from the at least one synthesis gas, and the methanol synthesis unit being configured to produce a second output from the at least one synthesis gas, wherein the selection of the liquid fuels production unit is determined by the mixed-integer linear optimization model solved by the global optimization framework;
a liquid fuels upgrading unit configured to upgrade the first output of the second output, wherein the type of liquid fuels upgrading unit is determined by the mixed-integer linear optimization model solved by the global optimization framework;
a hydrogen production unit configured to produce hydrogen for the refinery;
an oxygen production unit configured to produce oxygen for the refinery;
a wastewater treatment network configured to process wastewater from the refinery and input freshwater into the refinery, wherein the type of wastewater treatment network is determined by a mixed-integer linear optimization model solved by a global optimization framework;
a utility plant configured to produce electricity for the refinery and process heat from the refinery, wherein the type of utility plant is determined by a mixed-integer linear optimization model solved by a global optimization framework; and
a CO₂ separation unit configured to recylce gases containing CO₂ in the refinery, wherein
the at least one synthesis gas production unit, the synthesis gas cleanup unit, the liquid fuels production unit, the liquid fuels upgrading unit, the hydrogen production unit, the oxygen production unit, the wastewater treatment network, and the utility plant and the CO₂ separation unit are configured to be combined to form the refinery.

2. The superstructure of claim 1, wherein the biomass synthesis gas production unit is a biomass gasification unit.

3. The superstructure of claim 1, wherein the coal synthesis gas production unit is a coal gasification unit.

4. The superstructure of claim 1, wherein the natural gas synthesis gas production unit is a natural gas auto-thermal reforming unit.

5. The superstructure of claim 1, wherein the synthesis gas cleanup unit includes a hydrolyzer, a scrubber, a rectisol unit, a strupper column, and a claus recovery system.

6. The superstructure of claim 1, wherein the liquid fuels production unit is the Fischer-Tropsch unit.

7. The superstructure of claim 6, wherein the Fischer-Tropsch unit is selected from the group consisting of a low temperature cobalt catalyst Fischer-Tropsch unit; a high temperature cobalt catalyst Fischer-Tropsch unit; a medium temperature low wax iron catalyst Fischer-Tropsch unit; a medium temperature high wax iron catalyst Fischer-Tropsch unit; a high temperature iron catalyst Fischer-Tropsch unit; and a low temperature iron catalyst Fischer-Tropsch unit.

8. The superstructure of claim 7, wherein the liquid fuels upgrading unit is a ZSM-5 catalytic reactor.

9. The superstructure of claim 7, wherein the liquid fuels upgrading unit is a series of hydrotreating units, a wax hydrocracker, two isomerization units, a naphtha reformer, an alkylation unit and a gas separation plant.

10. The superstructure of claim 1, wherein the liquid fuels production unit is the methanol synthesis unit.

11. The superstructure of claim 10, wherein the liquid fuels upgrading unit is a methanol-to-gasoline reactor.

12. The superstructure of claim 10, wherein the liquid fuels upgrading unit is a methanol-to-olefins reactor and a Mobil olefins-to-gasoline/distillate reactor.

13. The superstructure of claim 1, wherein the hydrogen production unit is a pressure swing adsorption unit.

14. The superstructure of claim 1, wherein the hydrogen production unit is an electrolyzer unit.

15. The superstructure of claim 1, wherein the oxygen production unit is an electrolyzer unit.

16. The superstructure of claim 1, wherein the oxygen production unit is a distinct air separation unit.

17. The superstructure of claim 1, wherein the utility plant includes a gas turbine, a steam turbine, and a series of heat exchangers.

18. A refinery design system comprising:
a superstructure database, the superstructure database comprising data associated with:
at least one synthesis gas production unit configured to produce at least one synthesis gas selected from the group consisting of a biomass synthesis gas production unit, a coal synthesis gas production unit and a natural gas synthesis gas production unit, wherein the selection of the at least one synthesis gas production unit is determined by a mixed-integer linear optimization model solved by a global optimization framework;
a synthesis gas cleanup unit configured to remove undesired gases from the at least one synthesis gas;
a liquid fuels production unit selected from the group consisting of a Fischer-Tropsch unit and a methanol synthesis unit, the Fischer-Tropsch unit being configured to produce a first output from the at least one synthesis gas, and the methanol synthesis unit being configured to produce a second output from the at least one synthesis gas, wherein the selection of the liquid fuels production unit is determined by the mixed-integer linear optimization model solved by the global optimization framework;
a liquid fuels upgrading unit configured to upgrade the first output or the second output, wherein the type of liquid fuels upgrading unit is determined by the mixed-integer linear optimization model solved by the global optimization framework;
a hydrogen production unit configured to produce hydrogen for the refinery;
an oxygen production unit configured to produce oxygen for the refinery;
a wastewater treatment network configured to process wastewater from the refinery and input freshwater into the refinery, wherein the wastewater treatment network is determined by the mixed-integer linear optimization model solved by the global optimization framework;
a utility plant configured to produce electricity for the refinery and process heat from the refinery, wherein the type of utility plant is determined by the mixed-integer linear optimization model solved by the global optimization framework;
a CO₂ separation unit configured to recycle gases containing CO₂ in the refinery, wherein
the at least one synthesis gas production unit, the synthesis gas cleanup unit, the liquid fuels production unit, the liquid fuels upgrading unit, the hydrogen production unit, the oxygen production unit, the wastewater treatment network, the utility plant and the CO₂ separation unit are configured to be combined to form the refinery; and
a processor configured to solve the mixed-integer linear optimization model by the global optimization framework.

19. The refinery design system of claim 18, wherein the biomass synthesis gas production unit is a biomass gasification unit.

20. The refinery design system of claim 18, wherein the coal synthesis gas production unit is generated a coal gasification unit.

21. The refinery design system of claim 18, wherein the natural gas synthesis gas production unit is a natural gas auto-thermal reforming unit.

22. The refinery design system of claim 18, wherein the synthesis gas cleanup unit includes a hydrolyzer, a scrubber, a rectisol unit, a strupper column, and a claus recovery system.

23. The refinery design system of claim 18, wherein the liquid fuels production unit is the Fischer-Tropsch unit.

24. The refinery design system of claim 23, wherein the Fischer-Tropsch unit is selected from the group consisting of a low temperature cobalt catalyst Fischer-Tropsch unit; a high temperature cobalt catalyst Fischer-Tropsch unit; a medium temperature low wax iron catalyst Fischer-Tropsch unit; a medium temperature high wax iron catalyst Fischer-Tropsch; a high temperature iron catalyst Fischer-Tropsch unit; and a low temperature iron catalyst Fischer-Tropsch unit.

25. The refinery design system of claim 24, wherein the liquid fuels upgrading unit is a ZSM-5 catalytic reactor.

26. The refinery design system of claim 28, wherein the liquid fuels upgrading unit is a series of hydrotreating units, a wax hydrocracker, two isomerization units, a naphtha reformer, an alkylation unit and a gas separation plant.

27. The refinery design system of claim 18, wherein the liquid fuels production unit is the methanol synthesis unit.

28. The refinery design system of claim 27, wherein the liquid fuels upgrading unit is a methanol-to-gasoline reactor.

29. The refinery design system of claim 27, wherein the liquid fuels upgrading unit is a methanol-to-olefins reactor and a mobil olefins-to-gasoline/distillate reactor.

30. The refinery design system of claim 18, wherein the hydrogen production unit is a pressure swing adsorption unit.

31. The refinery design system of claim 18, wherein the hydrogen production unit is an electrolyzer unit.

32. The refinery design system of claim 18, wherein the oxygen production unit is an electrolyzer unit.

33. The refinery design system of claim 18, wherein the oxygen production unit is a distinct air separation unit.

34. The refinery design system of claim 18, wherein the utility plant includes a gas turbine, a steam turbine, and a series of heat exchangers.

35. A method of designing a refinery comprising:
providing the superstructure of claim 1;
inserting a data set on each of the at least one synthesis gas production unit, the liquid fuels production unit, the liquid fuels upgrading unit, the wastewater treatment network and the utility plant into the mixed-integer linear optimization model;
solving the mixed-integer linear optimization model by the global optimization framework; and
determining each of the at least one synthesis gas production unit, the liquid fuels production unit, the liquid fuels upgrading unit, the wastewater treatment network and the utility plant to produce an optimal refinery design.

36. A method of designing a refinery comprising:
providing the superstructure database of claim 18;
solving the mixed-integer linear optimization model by the global optimization framework; and
determining each of the at least one synthesis gas production unit, the liquid fuels production unit, the liquid fuels upgrading unit, the wastewater treatment network and the utility plant to produce an optimal refinery design.

37. A method of producing liquid fuels comprising:
producing liquid fuels with a refinery having a refinery design arrived at by
providing the superstructure of claim 1;
inserting a data set on each of the at least one synthesis gas production unit, the liquid fuels production unit, the liquid fuels upgrading unit, the wastewater treatment network and the utility plant into the mixed-integer linear optimization model;
solving the mixed-integer linear optimization model by the global optimization framework; and
determining each of the at least one synthesis gas production unit, the liquid fuels production unit, the liquid fuels upgrading unit, the wastewater treatment network and the utility plant to produce an optimal refinery design.

38. A method of producing liquid fuels comprising:
providing the superstructure database of any of claim 18;
solving the mixed-integer linear optimization model by the global optimization framework;
determining each of the at least one synthesis gas production unit, the liquid fuels production unit, the liquid fuels upgrading unit, the wastewater treatment network and the utility plant to produce a refinery design; and
producing liquid fuels by the refinery design.

39-43. (canceled)
